(19) United States
(12) Patent Application Publication
(10) Pub. No.: US 2025/0127809 A1
(43) Pub. Date: Apr. 24, 2025

(54) COMPOSITIONS AND METHODS FOR THE TREATMENT OF HEMOGLOBINOPATHIES

(71) Applicant: Novartis AG, Basel (CH)

(72) Inventors: Muluken Shambel BELEW, Ontario (CA); Jennifer Stroka COBB, Stow, MA (US); Carsten RUSS, Arlington, MA (US); Frederic SIGOILLOT, Waltham, MA (US); Susan C. STEVENSON, Ashland, MA (US); Pamela YF TING, SOMERVILLE, MA (US)

(21) Appl. No.: 18/569,029

(22) PCT Filed: Jun. 22, 2022
(86) PCT No.: PCT/IB2022/055799

§ 371 Date: Dec. 11, 2023

Related U.S. Application Data
(60) Provisional application No. 63/214,070, filed on Jun. 23, 2021.

(51) Int. Cl. A61K 35/14; A61K 35/12; A61P 7/00; C12N 5/071; C12N 9/22; C12N 15/113; C12N 15/52

(52) U.S. Cl. CPC A61K 35/14; A61P 7/00; C12N 5/0602; C12N 9/22; C12N 15/113; C12N 15/52; A61K 2035/124; C12N 2510/00

(57) ABSTRACT

The present invention is directed to compositions and methods for the treatment of hemoglobinopathies.

### CROSS-REFERENCE TO RELATED APPLICATION

This application claims the benefit of and priority to the following U.S. Provisional Application No. 63/214,070, filed Jun. 23, 2021, the entire contents of which are incorporated herein by reference.

### SEQUENCE LISTING

The instant application contains a Sequence Listing, which has been submitted electronically in ASCII format and is hereby incorporated by reference in its entirety. Said ASCII copy, created on Jun. 21, 2022, is named PAT059150-WO-PCT_SL.txt and is 895,430 bytes in size.

### BACKGROUND

CRISPRs (Clustered Regularly Interspaced Short Palindromic Repeats) evolved in bacteria as an adaptive immune system to defend against viral attack. Upon exposure to a virus, short segments of viral DNA are integrated into the CRISPR locus of the bacterial genome. RNA is transcribed from a portion of the CRISPR locus that includes the viral sequence. That RNA, which contains sequence that is complimentary to the viral genome, mediates targeting of a Cas9 protein to the sequence in the viral genome. The Cas9 protein cleaves and thereby silences the viral target.

Recently, the CRISPR/Cas system has been adapted for genome editing in eukaryotic cells. The introduction of site-specific single (SSBs) or double strand breaks (DSBs) allows for target sequence alteration through, for example, non-homologous end-joining (NHEJ) or homology-directed repair (HDR).

### SUMMARY OF THE INVENTION

Without being bound by theory, the invention here is based in part on the surprising finding of the linkage between ZNF644 gene expression/protein activity and the hemoglobin F (HbF) production. As demonstrated in the examples and figures, knocking down or knocking out ZNF644 gene or ZNF644 protein in cells (by various modalities/compositions described herein) significantly increased HbF induction in those cells, thereby treating HbF-associated conditions and disorders (e.g., hemoglobinopathies, e.g., sickle cell disease and beta thalassemia). The invention is also based in part on the discovery that CRISPR systems, e.g., Cas9 CRISPR systems, e.g., as described herein, can be used to modify cells (e.g., hematopoietic stem and progenitor cells (HSPCs)), for example, at ZNF644 gene, as described herein, to increase fetal hemoglobin (HbF) expression and/or decrease expression of beta globin (e.g., a beta globin gene having a disease-causing mutation), for example in progeny, for example red blood cell progeny, of the modified cells, and that the modified cells (e.g., modified HSPCs) may be used to treat hemoglobinopathies, e.g., sickle cell disease and beta thalassemia. In one aspect, it has surprisingly been shown herein that introduction of gene editing systems, e.g., CRISPR systems, e.g., as described herein, to cells (e.g., HSPCs), that target ZNF644 gene to create modified HSPCs (e.g., HSPCs that comprise one or more indels, for example, as described herein) that are able to efficiently engraft into an organism, persist long-term in the engrafted organism, and differentiate, including into erythrocytes with increased fetal hemoglobin expression. In addition, these modified HSPCs are capable of being cultured ex vivo, for example, in the presence of a stem cell expander (for example as described herein) under conditions that cause them to expand and proliferate while maintaining stemness. When the gene editing systems, e.g., CRISPR systems, e.g., as described herein, are introduced into HPSCs derived from sickle cell disease patients, the modified cells and their progeny (e.g., erythroid progeny) surprisingly show not only upregulation of fetal hemoglobin, but also show a significant decrease in sickle beta-globin, and a significant decrease in the number of sickle cells and increase the number of normal red blood cells, relative to unmodified cell populations.

Thus, in an aspect, the invention provides CRISPR systems (e.g., Cas CRISPR systems, e.g., Cas9 CRISPR systems, e.g., S. pyogenes Cas9 CRISPR systems) comprising one or more, e.g., one, gRNA molecule as described herein. Any of the gRNA molecules described herein may be used in such systems, and in the methods and cells described herein.

In an aspect, the invention provides a gRNA molecule including a tracr and crRNA, wherein the crRNA includes a targeting domain that is complementary with a target sequence of ZNF644 gene (e.g., a human ZNF644 gene). In embodiments, the ZNF644 gene includes genomic nucleic acid sequence at Chr19:15419978-15451624, − strand, hg38, or a fragment thereof or a variant thereof. In embodiments, the targeting domain includes, e.g., consists of, any one of SEQ ID NO: 1 to SEQ ID NO: 2613 (see, e.g., Tables 1-3). In embodiments, the gRNA molecule includes a targeting domain which includes (e.g., consists of) a fragment of any of the sequences above.

In any of the aspects and embodiments described herein, the gRNA molecule may further have regions and/or properties described herein. In embodiments, the gRNA molecule includes a fragment of any of the targeting domains described herein. In embodiments, the targeting domain includes, e.g., consists of, 17, 18, 19, or 20 consecutive nucleic acids of any one of the recited targeting domain sequences. In embodiments, the 17, 18, 19, or 20 consecutive nucleic acids of any one of the recited targeting domain sequences are the 17, 18, 19, or 20 consecutive nucleic acids disposed at the 3′ end of the recited targeting domain sequence. In other embodiments, the 17, 18, 19, or 20 consecutive nucleic acids of any one of the recited targeting domain sequences are the 17, 18, 19, or 20 consecutive nucleic acids disposed at the 5′ end of the recited targeting domain sequence. In other embodiments, the 17, 18, 19, or 20 consecutive nucleic acids of any one of the recited targeting domain sequences do not include either the 5′ or 3′ nucleic acid of the recited targeting domain sequence. In embodiments, the targeting domain consists of the recited targeting domain sequence.

In an aspect, including in any of the aspects and embodiments described herein, a portion of the crRNA and a portion of the tracr hybridize to form a flagpole including SEQ ID NO: 3110 or 3111. In an aspect, including in any of the aspects and embodiments described herein, the flagpole further includes a first flagpole extension, located 3′ to the crRNA portion of the flagpole, wherein said first flagpole extension includes SEQ ID NO: 3112. In an aspect, including in any of the aspects and embodiments described herein, the flagpole further includes a second flagpole extension located 3′ to the crRNA portion of the flagpole and, if present, the first flagpole extension, wherein said second flagpole extension includes SEQ ID NO: 3113.

In an aspect, including in any of the aspects and embodiments described herein, the tracr includes SEQ ID NO: 3152 or SEQ ID NO: 3153. In an aspect, including in any of the aforementioned aspects and embodiments, the tracr includes SEQ ID NO: 3160, optionally further including, at the 3′ end, an additional 1, 2, 3, 4, 5, 6, or 7 uracil (U) nucleotides. In an aspect, including in any of the aspects and embodiments described herein, the crRNA includes, from 5′ to 3′, [targeting domain]-: a) SEQ ID NO:3110; b) SEQ ID NO: 3111; c) SEQ ID NO: 3127; d) SEQ ID NO: 3128; e) SEQ ID NO: 3129; f) SEQ ID NO: 3130; or g) SEQ ID NO: 3154.

In an aspect, including in any of the aforementioned aspects and embodiments, the tracr includes, from 5′ to 3′: a) SEQ ID NO: 3115; b) SEQ ID NO: 3116; c) SEQ ID NO: 3131; d) SEQ ID NO: 3132; e) SEQ ID NO: 3152; f) SEQ ID NO: 3153; g) SEQ ID NO: 3160; h) SEQ ID NO: 3155; i) (SEQ ID NO: 3156; j) SEQ ID NO: 3157; k) any of a) to j), above, further including, at the 3′ end, at least 1, 2, 3, 4, 5, 6 or 7 uracil (U) nucleotides, e.g., 1, 2, 3, 4, 5, 6, or 7 uracil (U) nucleotides; 1) any of a) to k), above, further including, at the 3′ end, at least 1, 2, 3, 4, 5, 6 or 7 adenine (A) nucleotides, e.g., 1, 2, 3, 4, 5, 6, or 7 adenine (A) nucleotides; or m) any of a) to 1), above, further including, at the 5′ end (e.g., at the 5′ terminus), at least 1, 2, 3, 4, 5, 6 or 7 adenine (A) nucleotides, e.g., 1, 2, 3, 4, 5, 6, or 7 adenine (A) nucleotides.

In an aspect, including in any of the aspects and embodiments described herein, the targeting domain and the tracr are disposed on separate nucleic acid molecules. In an aspect, including in any of the aspects and embodiments described herein, the targeting domain and the tracr are disposed on separate nucleic acid molecules, and the nucleic acid molecule including the targeting domain includes SEQ ID NO: 3129, optionally disposed immediately 3′ to the targeting domain, and the nucleic acid molecule including the tracr includes, e.g., consists of, SEQ ID NO: 3152. In an aspect, including in any of the aforementioned aspects and embodiments, the crRNA portion of the flagpole includes SEQ ID NO: 3129 or SEQ ID NO: 3130. In an aspect, including in any of the aforementioned aspects and embodiments, the tracr includes SEQ ID NO: 3115 or 3116, and optionally, if a first flagpole extension is present, a first tracr extension, disposed 5′ to SEQ ID NO: 3115 or 3116, said first tracr extension including SEQ ID NO: 3117.

In an aspect, including in any of the aforementioned aspects and embodiments, the targeting domain and the tracr are disposed on a single nucleic acid molecule, for example, wherein the tracr is disposed 3′ to the targeting domain. In an aspect, the gRNA molecule includes a loop, disposed 3′ to the targeting domain and 5′ to the tracr. In embodiments, the loop includes SEQ ID NO: 3114. In an aspect, including in any of the aforementioned aspects and embodiments, the gRNA molecule includes, from 5′ to 3′, [targeting domain]-: (a) SEQ ID NO: 3123; (b) SEQ ID NO: 3124; (c) SEQ ID NO: 3125; (d) SEQ ID NO: 3126; (e) SEQ ID NO: 3159; or (f) any of (a) to (e), above, further including, at the 3′ end, 1, 2, 3, 4, 5, 6 or 7 uracil (U) nucleotides.

In an aspect, including in any of the aforementioned aspects and embodiments, the targeting domain and the tracr are disposed on a single nucleic acid molecule, and wherein said nucleic acid molecule includes, e.g., consists of, said targeting domain and SEQ ID NO: 3159, optionally disposed immediately 3′ to said targeting domain.

In an aspect, including in any of the aforementioned aspects and embodiments, one, or optionally more than one, of the nucleic acid molecules including the gRNA molecule includes:
a) one or more, e.g., three, phosphorothioate modifications at the 3′ end of said nucleic acid molecule or molecules;
b) one or more, e.g., three, phosphorothioate modifications at the 5′ end of said nucleic acid molecule or molecules;
c) one or more, e.g., three, 2′-O-methyl modifications at the 3′ end of said nucleic acid molecule or molecules;
d) one or more, e.g., three, 2′-O-methyl modifications at the 5′ end of said nucleic acid molecule or molecules;
e) a 2′ O-methyl modification at each of the 4^{th}-to-terminal, 3^{rd}-to-terminal, and 2^{nd}-to-terminal 3′ residues of said nucleic acid molecule or molecules;
f) a 2′ O-methyl modification at each of the 4^{th}-to-terminal, 3^{rd}-to-terminal, and 2^{nd}-to-terminal 5′ residues of said nucleic acid molecule or molecules; or
f) any combination thereof.

In an aspect, including in any of the aforementioned aspects and embodiments the invention provides a gRNA molecule, wherein: when a CRISPR system (e.g., an RNP as described herein) including the gRNA molecule is introduced into a cell, an indel is formed at or near the target sequence complementary to the targeting domain of the gRNA molecule.

In an aspect, including in any of the aforementioned aspects and embodiments, the invention provides a gRNA molecule, wherein when a CRISPR system (e.g., an RNP as described herein) including the gRNA molecule is introduced into a population of cells, an indel is formed at or near the target sequence complementary to the targeting domain of the gRNA molecule in at least about 15%, e.g., at least about 17%, e.g., at least about 20%, e.g., at least about 30%, e.g., at least about 40%, e.g., at least about 50%, e.g., at least about 55%, e.g., at least about 60%, e.g., at least about 70%, e.g., at least about 75%, of the cells of the population. In an aspect, including in any of the aforementioned aspects and embodiments, the indel includes at least one nucleotide of a ZNF644 gene region. In embodiments, at least about 15% of the cells of the population include an indel which includes at least one nucleotide of a ZNF644 gene region. In embodiments, the indel is as measured by next generation sequencing (NGS).

In an aspect, including in any of the aforementioned aspects and embodiments, the invention provides a gRNA molecule, wherein when a CRISPR system (e.g., an RNP as described herein) including the gRNA molecule is introduced into a cell, expression of fetal hemoglobin is increased in said cell or its progeny, e.g., its erythroid progeny, e.g., its red blood cell progeny. In embodiments, when a CRISPR system (e.g., an RNP as described herein) including the gRNA molecule is introduced into a population of cells, the percentage of F cells in said population or population of its progeny, e.g., its erythroid progeny, e.g., its red blood cell progeny, is increased by at least about 15%, e.g., at least about 17%, e.g., at least about 20%, e.g., at least about 25%, e.g., at least about 30%, e.g., at least about 35%, e.g., at least about 40%, relative to the percentage of F cells in a population of cells to which the gRNA molecule was not introduced or a population of its progeny, e.g., its erythroid progeny, e.g., its red blood cell progeny. In embodiments, said cell or its progeny, e.g., its erythroid progeny, e.g., its red blood cell progeny, produces at least about 6 picograms (e.g., at least about 7 picograms, at least about 8 picograms, at least about 9 picograms, at least about 10 picograms, or from about 8 to about 9 picograms, or from about 9 to about 10 picograms) fetal hemoglobin per cell.

In an aspect, including in any of the aforementioned aspects and embodiments, the invention provides a gRNA molecule, wherein when a CRISPR system (e.g., an RNP as described herein) including the gRNA molecule is introduced into a cell, no off-target indels are formed in said cell, e.g., no off-target indels are formed outside of the ZNF644 gene region (e.g., within a gene, e.g., a coding region of a gene), e.g., as detectible by next generation sequencing and/or a nucleotide insertional assay. In an aspect, including in any of the aforementioned aspects and embodiments, the invention provides a gRNA molecule, wherein when a CRISPR system (e.g., an RNP as described herein) including the gRNA molecule is introduced into a population of cells, no off-target indel, e.g., no off-target indel outside of the ZNF644 gene (e.g., within a gene, e.g., a coding region of a gene), is detected in more than about 5%, e.g., more than about 1%, e.g., more than about 0.1%, e.g., more than about 0.01%, of the cells of the population of cells, e.g., as detectible by next generation sequencing and/or a nucleotide insertional assay.

In an aspect, including of any of the aforementioned aspects and embodiments, the cell is (or population of cells includes) a mammalian, primate, or human cell, e.g., is a human cell, e.g., the cell is (or population of cells includes) an HSPC, e.g., the HSPC is CD34+, e.g., the HSPC is CD34+CD90+. In embodiments, the cell is autologous with respect to a patient to be administered said cell. In other embodiments, the cell is allogeneic with respect to a patient to be administered said cell.

In an aspect, the gRNA molecules, genome editing systems (e.g., CRISPR systems), and/or methods described herein relate to cells, e.g., as described herein, that include or result in one or more of the following properties:
(a) at least about 40%, at least about 50%, at least about 60%, at least about 70%, at least about 80%, at least about 90%, at least about 95%, at least about 96%, at least about 97%, at least about 98% or at least about 99% of the cells of a population of cells described herein comprise an indel at or near a genomic DNA sequence complementary to the targeting domain of a gRNA molecule described herein;
(b) a cell (e.g., population of cells) described herein is capable of differentiating into a differentiated cell of an erythroid lineage (e.g., a red blood cell), and wherein said differentiated cell exhibits an increased level of fetal hemoglobin, e.g., relative to an unaltered cell (e.g., population of cells);
(c) a population of cells described herein is capable of differentiating into a population of differentiated cells, e.g., a population of cells of an erythroid lineage (e.g., a population of red blood cells), and wherein said population of differentiated cells has an increased percentage of F cells (e.g., at least about 15%, at least about 20%, at least about 25%, at least about 30%, or at least about 40% higher percentage of F cells) e.g., relative to a population of unaltered cells;
(d) a cell (e.g., population of cells) described herein is capable of differentiating into a differentiated cell, e.g., a cell of an erythroid lineage (e.g., a red blood cell), and wherein said differentiated cell (e.g., population of differentiated cells) produces at least about 6 picograms (e.g., at least about 7 picograms, at least about 8 picograms, at least about 9 picograms, at least about 10 picograms, or from about 8 to about 9 picograms, or from about 9 to about 10 picograms) fetal hemoglobin per cell;
(e) no off-target indels are formed in a cell described herein, e.g., no off-target indels are formed outside of the ZNF644 gene region (e.g., within a gene, e.g., a coding region of a gene), e.g., as detectible by next generation sequencing and/or a nucleotide insertional assay;
(f) no off-target indel, e.g., no off-target indel outside of the ZNF644 gene region (e.g., within a gene, e.g., a coding region of a gene), is detected in more than about 5%, e.g., more than about 1%, e.g., more than about 0.1%, e.g., more than about 0.01%, of the cells of a population of cells described herein, e.g., as detectible by next generation sequencing and/or a nucleotide insertional assay;
(g) a cell described herein or its progeny is detectable, e.g., detectable in the bone marrow or detectable in the peripheral blood, in a patient to which it is transplanted at more than 16 weeks, more than 20 weeks or more than 24 weeks after transplantation, optionally as detected by detecting an indel at or near a genomic DNA sequence complementary to the targeting domain of a gRNA molecule of any of SEQ ID NO: 1 to SEQ ID NO: 2613, optionally wherein the indel is a large deletion indel;
(h) a population of cells described herein is capable of differentiating into a population of differentiated cells, e.g., a population of cells of an erythroid lineage (e.g., a population of red blood cells), and wherein said population of differentiated cells includes a reduced percentage of sickle cells (e.g., at least about 15%, at least about 20%, at least about 25%, at least about 30%, at least about 40%, at least about 50%, at least about 60%, at least about 70%, at least about 80%, or at least about 90% lower percentage of sickle cells) e.g., relative to a population of unaltered cells; and/or
(i) a cell or population of cells described herein is capable of differentiating into a population of differentiated cells, e.g., a population of cells of an erythroid lineage (e.g., a population of red blood cells), and wherein said population of differentiated cells includes cells which produce a reduced level (e.g., at least about 15%, at least about 20%, at least about 25%, at least about 30%, at least about 40%, at least about 50%, at least about 60%, at least about 70%, at least about 80%, or at least about 90% lower level) of sickle hemoglobin (HbS), e.g., relative to a population of unaltered cells.

In an aspect, the invention provides a composition including:
1) one or more gRNA molecules (including a first gRNA molecule) described herein, e.g., of any of the aforementioned gRNA aspects and embodiments, and a Cas9 molecule, e.g., described herein;
2) one or more gRNA molecules (including a first gRNA molecule) described herein, e.g., of any of the aforementioned gRNA aspects and embodiments, and nucleic acid comprising a nucleotide sequence encoding a Cas9 molecule, e.g., described herein;
3) nucleic acid comprising one or more nucleotide sequences each encoding one gRNA molecule (including a first gRNA molecule) described herein, e.g., of any of the aforementioned gRNA aspects and embodiments, and a Cas9 molecule, e.g., described herein;
4) nucleic acid comprising one or more nucleotide sequences each encoding one gRNA molecule (including a first gRNA molecule) described herein, e.g., of any of the aforementioned gRNA aspects and embodiments, and nucleic acid encoding a Cas9 molecule, e.g., described herein; or 5) any of 1) to 4), above, and a template nucleic acid; or
6) any of 1) to 4) above, and nucleic acid including a nucleotide sequence encoding a template nucleic acid.

In an aspect, the invention provides a composition including a first gRNA molecule described herein, e.g., of any of the aforementioned gRNA aspects and embodiments, further including a Cas9 molecule, e.g., described herein, e.g., wherein the Cas9 molecule is an active or inactive s. pyogenes Cas9, for example, wherein the Cas9 molecule includes SEQ ID NO: 3133. In aspects, the Cas9 molecule includes, e.g., consists of: (a) SEQ ID NO: 3161; (b) SEQ ID NO: 3162; (c) SEQ ID NO: 3163; (d) SEQ ID NO: 3164; (e) SEQ ID NO: 3165; (f) SEQ ID NO: 3166; (g) SEQ ID NO: 3167; (h) SEQ ID NO: 3168; (i) SEQ ID NO: 3169; (j) SEQ ID NO: 3170; (k) SEQ ID NO: 3171 or (1) SEQ ID NO: 3172.

In an aspect, including in any of the aforementioned composition aspects and embodiments, the first gRNA molecule and Cas9 molecule are present in a ribonuclear protein complex (RNP).

In an aspect, including in any of the aforementioned composition aspects and embodiments, the invention provides a composition further including a second gRNA molecule; a second gRNA molecule and a third gRNA molecule; or a second gRNA molecule, optionally, a third gRNA molecule, and, optionally, a fourth gRNA molecule, wherein the second gRNA molecule, the optional third gRNA molecule, and the optional fourth gRNA molecule are a gRNA molecule described herein, e.g., are a gRNA molecule of any of the aforementioned gRNA molecule aspects and embodiments, and wherein each gRNA molecule of the composition is complementary to a different target sequence. In embodiments, two or more of the first gRNA molecule, the second gRNA molecule, the optional third gRNA molecule, and the optional fourth gRNA molecule are complementary to target sequences within the same gene or region. In embodiments, the first gRNA molecule, the second gRNA molecule, the optional third gRNA molecule, and the optional fourth gRNA molecule are complementary to target sequences not more than 6000 nucleotides, not more than 5000 nucleotides, not more than 500, not more than 400 nucleotides, not more than 300, not more than 200 nucleotides, not more than 100 nucleotides, not more than 90 nucleotides, not more than 80 nucleotides, not more than 70 nucleotides, not more than 60 nucleotides, not more than 50 nucleotides, not more than 40 nucleotides, not more than 30 nucleotides, not more than 20 nucleotides or not more than 10 nucleotides apart. In an aspect, including in any of the aforementioned composition aspects and embodiments, the composition includes (e.g., consists of) a first gRNA molecule and a second gRNA molecule, wherein the first gRNA molecule and second gRNA molecule are: (a) independently selected and are complementary to different target sequences; (b) independently selected from the gRNA molecules of Table 1, and are complementary to different target sequences; c) independently selected from the gRNA molecules of Table 2, and are complementary to different target sequences; or (d) independently selected from the gRNA molecules of Table 3 and are complementary to different target sequences, or (f) independently selected from the gRNA molecules of any of the aforementioned aspects and embodiments, and are complementary to different target sequences.

In an aspect, including in any of the aforementioned composition aspects and embodiments, the composition includes a first gRNA molecule and a second gRNA molecule, wherein:
a) the first gRNA molecule is complementary to a target sequence including at least 1 nucleotide (e.g., including 20 consecutive nucleotides) within: Chr19:15419978-15451624, − strand, hg38;
b) the second gRNA molecule is complementary to a target sequence including at least 1 nucleotide (e.g., comprising 20 consecutive nucleotides) within: Chr19:15419978-15451624, − strand, hg38.

In an aspect, with respect to the gRNA molecule components of the composition, the composition consists of a first gRNA molecule and a second gRNA molecule.

In an aspect, including in any of the aforementioned composition aspects and embodiments, each of said gRNA molecules is in a ribonuclear protein complex (RNP) with a Cas9 molecule, e.g., described herein.

In an aspect, including in any of the aforementioned composition aspects and embodiments, the composition includes a template nucleic acid, wherein the template nucleic acid includes a nucleotide that corresponds to a nucleotide at or near the target sequence of the first gRNA molecule. In embodiments, the template nucleic acid includes nucleic acid encoding: human ZNF644 gene, or fragment thereof.

In an aspect, including in any of the aforementioned composition aspects and embodiments, the composition is formulated in a medium suitable for electroporation.

In an aspect, including in any of the aforementioned composition aspects and embodiments, each of said gRNA molecules of said composition is in a RNP with a Cas9 molecule described herein, and wherein each of said RNP is at a concentration of less than about 10 uM, e.g., less than about 3 uM, e.g., less than about 1 uM, e.g., less than about 0.5 uM, e.g., less than about 0.3 uM, e.g., less than about 0.1 uM. In embodiments, the RNP is at a concentration of about 1 uM. In embodiments, the RNP is at a concentration of about 2 uM. In embodiments, said concentration is the concentration of RNP in a composition comprising the cells, e.g., as described herein, optionally wherein the composition comprising the cells and the RNP is suitable for electroporation.

In an aspect, the invention provides a nucleic acid sequence that encodes one or more gRNA molecules described herein, e.g., of any of the aforementioned gRNA molecule aspects and embodiments. In embodiments, the nucleic acid includes a promoter operably linked to the sequence that encodes the one or more gRNA molecules, for example, the promoter is a promoter recognized by an RNA polymerase II or RNA polymerase III, or, for example, the promoter is a U6 promoter or an HI promoter.

In an aspect, including in any of the aforementioned nucleic acid aspects and embodiments, the nucleic acid further encodes a Cas9 molecule, for example, a Cas9 molecule that includes, e.g., consists of, any of SEQ ID NO: 3133, (a) SEQ ID NO: 3161; (b) SEQ ID NO: 3162; (c) SEQ ID NO: 3163; (d) SEQ ID NO: 3164; (e) SEQ ID NO: 3165; (f) SEQ ID NO: 3166; (g) SEQ ID NO: 3167; (h) SEQ ID NO: 3168; (i) SEQ ID NO: 3169; (j) SEQ ID NO: 3170; (k) SEQ ID NO: 3171 or (1) SEQ ID NO: 3172. In embodiments, said nucleic acid includes a promoter operably linked to the sequence that encodes a Cas9 molecule, for example, an EF-1 promoter, a CMV IE gene promoter, an EF-1a promoter, an ubiquitin C promoter, or a phosphoglycerate kinase (PGK) promoter.

In an aspect, provided herein includes a vector including the nucleic acid of any of the aforementioned nucleic acid aspects and embodiments. In embodiments, the vector is selected from the group consisting of a lentiviral vector, an adenoviral vector, an adeno-associated viral (AAV) vector, a herpes simplex virus (HSV) vector, a plasmid, a minicircle, a nanoplasmid, and an RNA vector.

In an aspect, provided herein includes a method of altering a cell (e.g., a population of cells), (e.g., altering the structure (e.g., sequence) of nucleic acid) at or near a target sequence within said cell, including contacting (e.g., introducing into) said cell (e.g., population of cells) with:
1) one or more gRNA molecules described herein (e.g., of any of the aforementioned gRNA molecule aspects and embodiments) and a Cas9 molecule, e.g., described herein;
2) one or more gRNA molecules described herein (e.g., of any of the aforementioned gRNA molecule aspects and embodiments) and nucleic acid comprising a nucleotide sequence encoding a Cas9 molecule, e.g., described herein;
3) nucleic acid comprising one or more nucleotide sequences each encoding one gRNA molecule described herein (e.g., of any of the aforementioned gRNA molecule aspects and embodiments) and a Cas9 molecule, e.g., described herein;
4) nucleic acid comprising one or more nucleotide sequences each encoding one gRNA molecule described herein (e.g., of any of the aforementioned gRNA molecule aspects and embodiments) and nucleic acid comprising a nucleotide sequence encoding a Cas9 molecule, e.g., described herein;
5) any of 1) to 4), above, and a template nucleic acid;
6) any of 1) to 4) above, and nucleic acid including a nucleotide sequence encoding a template nucleic acid;
7) a composition described herein, e.g., a composition of any of the aforementioned composition aspects and embodiments; or
8) a vector described herein, e.g., a vector of any of the aforementioned vector aspects and embodiments.

In an aspect, including in any of the aforementioned method aspects and embodiments, the gRNA molecule or nucleic acid encoding the gRNA molecule, and the Cas9 molecule or nucleic acid encoding the Cas9 molecule, are formulated in a single composition. In another aspect, the gRNA molecule or nucleic acid encoding the gRNA molecule, and the Cas9 molecule or nucleic acid encoding the Cas9 molecule, are formulated in more than one composition. In an aspect, the more than one composition are delivered simultaneously or sequentially.

In an aspect of the methods described herein, including in any of the aforementioned method aspects and embodiments, the cell is an animal cell, for example, the cell is a mammalian, primate, or human cell, for example, the cell is a hematopoietic stem or progenitor cell (HSPC) (e.g., a population of HSPCs), for example, the cell is a CD34+ cell, for example, the cell is a CD34+CD90+ cell. In embodiments of the methods described herein, the cell is disposed in a composition including a population of cells that has been enriched for CD34+ cells. In embodiments of the methods described herein, the cell (e.g. population of cells) has been isolated from bone marrow, mobilized peripheral blood, or umbilical cord blood. In embodiments of the methods described herein, the cell is autologous or allogeneic, e.g., autologous, with respect to a patient to be administered said cell.

In an aspect of the methods described herein, including in any of the aforementioned method aspects and embodiments, a) the altering results in an indel at or near a genomic DNA sequence complementary to the targeting domain of the one or more gRNA molecules; or b) the altering results in a deletion including sequence, e.g., substantially all the sequence, complementary to the targeting domain of the one or more gRNA molecules (e.g., at least 90% complementary to the gRNA targeting domain, e.g., fully complementary to the gRNA targeting domain) in the ZNF644 gene region. In aspects of the method, the indel is an insertion or deletion of less than about 40 nucleotides, e.g., less than 30 nucleotides, e.g., less than 20 nucleotides, e.g., less than 10 nucleotides, for example, is a single nucleotide deletion.

In an aspect of the methods described herein, including in any of the aforementioned method aspects and embodiments, the method results in a population of cells wherein at least about 15%, e.g., at least about 17%, e.g., at least about 20%, e.g., at least about 30%, e.g., at least about 40%, e.g., at least about 50%, e.g., at least about 55%, e.g., at least about 60%, e.g., at least about 70%, e.g., at least about 75% of the population have been altered, e.g., include an indel.

In an aspect of the methods described herein, including in any of the aforementioned method aspects and embodiments, the altering results in a cell (e.g., population of cells) that is capable of differentiating into a differentiated cell of an erythroid lineage (e.g., a red blood cell), and wherein said differentiated cell exhibits an increased level of fetal hemoglobin, e.g., relative to an unaltered cell (e.g., population of cells).

In an aspect of the methods described herein, including in any of the aforementioned method aspects and embodiments, the altering results in a population of cells that is capable of differentiating into a population of differentiated cells, e.g., a population of cells of an erythroid lineage (e.g., a population of red blood cells), and wherein said population of differentiated cells has an increased percentage of F cells (e.g., at least about 15%, at least about 20%, at least about 25%, at least about 30%, or at least about 40% higher percentage of F cells) e.g., relative to a population of unaltered cells. In an aspect of the methods described herein, including in any of the aforementioned method aspects and embodiments, the altering results in a cell that is capable of differentiating into a differentiated cell, e.g., a cell of an erythroid lineage (e.g., a red blood cell), and wherein said differentiated cell produces at least about 6 picograms (e.g., at least about 7 picograms, at least about 8 picograms, at least about 9 picograms, at least about 10 picograms, or from about 8 to about 9 picograms, or from about 9 to about 10 picograms) fetal hemoglobin per cell.

In an aspect, the invention provides a cell, altered by a method described herein, for example, a method of any of the aforementioned method aspects and embodiments.

In an aspect, the invention provides a cell, obtainable by a method described herein, for example, a method of any of the aforementioned method aspects and embodiments.

In an aspect, the invention provides a cell, including a first gRNA molecule described herein, e.g., of any of the aforementioned gRNA molecule aspects or embodiments, or a composition described herein, e.g., of any of the aforementioned composition aspects or embodiments, a nucleic acid described herein, e.g., of any of the aforementioned nucleic acid aspects or embodiments, or a vector described herein, e.g., of any of the aforementioned vector aspects or embodiments.

In an aspect of the cell described herein, including in any of the aforementioned cell aspects and embodiments, the cell further includes a Cas9 molecule, e.g., described herein, e.g., a Cas9 molecule that includes any one of SEQ ID NO: 3133, (a) SEQ ID NO: 3161; (b) SEQ ID NO: 3162; (c) SEQ ID NO: 3163; (d) SEQ ID NO: 3164; (e) SEQ ID NO: 3165; (f) SEQ ID NO: 3166; (g) SEQ ID NO: 3167; (h) SEQ ID NO: 3168; (i) SEQ ID NO: 3169; j) SEQ ID NO: 3170; (k) SEQ ID NO: 3171 or (1) SEQ ID NO: 3172.

In an aspect of the cell described herein, including in any of the aforementioned cell aspects and embodiments, the cell includes, has included, or will include a second gRNA molecule described herein, e.g., of any of the aforementioned gRNA molecule aspects or embodiments, or nucleic acid encoding said gRNA molecule, wherein the first gRNA molecule and second gRNA molecule include nonidentical targeting domains.

In an aspect of the cell described herein, including in any of the aforementioned cell aspects and embodiments, expression of fetal hemoglobin is increased in said cell or its progeny (e.g., its erythroid progeny, e.g., its red blood cell progeny) relative to a cell or its progeny of the same cell type that has not been modified to include a gRNA molecule.

In an aspect of the cell described herein, including in any of the aforementioned cell aspects and embodiments, the cell is capable of differentiating into a differentiated cell, e.g., a cell of an erythroid lineage (e.g., a red blood cell), and wherein said differentiated cell exhibits an increased level of fetal hemoglobin, e.g., relative to a cell of the same type that has not been modified to include a gRNA molecule.

In an aspect of the cell described herein, including in any of the aforementioned cell aspects and embodiments, the differentiated cell (e.g., cell of an erythroid lineage, e.g., red blood cell) produces at least about 6 picograms (e.g., at least about 7 picograms, at least about 8 picograms, at least about 9 picograms, at least about 10 picograms, or from about 8 to about 9 picograms, or from about 9 to about 10 picograms) fetal hemoglobin, e.g., relative to a differentiated cell of the same type that has not been modified to include a gRNA molecule.

In an aspect of the cell described herein, including in any of the aforementioned cell aspects and embodiments, the cell has been contacted, e.g., contacted ex vivo, with a stem cell expander, for example, a stem cell expander selected from: a) (1r,4r)-N¹-(2-benzyl-7-(2-methyl-2H-tetrazol-5-yl)-9H-pyrimido[4,5-b]indol-4-yl)cyclohexane-1,4-diamine; b) methyl 4-(3-piperidin-1-ylpropylamino)-9H-pyrimido[4,5-b]indole-7-carboxylate; c) 4-(2-(2-(benzo[b]thiophen-3-yl)-9-isopropyl-9H-purin-6-ylamino)ethyl)phenol; d) (S)-2-(6-(2-(1H-indol-3-yl)ethylamino)-2-(5-fluoropyridin-3-yl)-9H-purin-9-yl)propan-1-ol; or e) combinations thereof (e.g., a combination of (1r,4r)-N¹-(2-benzyl-7-(2-methyl-2H-tetrazol-5-yl)-9H-pyrimido[4,5-b]indol-4-yl)cyclohexane-1,4-diamine and (S)-2-(6-(2-(1H-indol-3-yl)ethylamino)-2-(5-fluoropyridin-3-yl)-9H-purin-9-yl)propan-1-ol). In embodiments, the stem cell expander is (S)-2-(6-(2-(1H-indol-3-yl)ethylamino)-2-(5-fluoropyridin-3-yl)-9H-purin-9-yl)propan-1-ol.

In an aspect of the cell described herein, including in any of the aforementioned cell aspects and embodiments, the cell includes: a) an indel at or near a genomic DNA sequence complementary to the targeting domain of a gRNA molecule described herein, e.g., of any of the aforementioned gRNA molecule aspects or embodiments; or b) a deletion including sequence, e.g., substantially all the sequence, complementary to the targeting domain of a gRNA molecule described herein, e.g., of any of the aforementioned gRNA molecule aspects or embodiments (e.g., at least 90% complementary to the gRNA targeting domain, e.g., fully complementary to the gRNA targeting domain) in the ZNF644 gene region. In an aspect, the indel is an insertion or deletion of less than about 40 nucleotides, e.g., less than 30 nucleotides, e.g., less than 20 nucleotides, e.g., less than 10 nucleotides, for example, the indel is a single nucleotide deletion.

In an aspect of the cell described herein, including in any of the aforementioned cell aspects and embodiments, the cell is an animal cell, for example, the cell is a mammalian, a primate, or a human cell. In an aspect, the cell is a hematopoietic stem or progenitor cell (HSPC) (e.g., a population of HSPCs), e.g., the cell is a CD34+ cell, e.g., the cell is a CD34+CD90+ cell. In embodiments, the cell (e.g. population of cells) has been isolated from bone marrow, mobilized peripheral blood, or umbilical cord blood. In embodiments, the cell is autologous with respect to a patient to be administered said cell. In embodiments, the cell the cell is allogeneic with respect to a patient to be administered said cell. In an aspect, the invention provides a population of cells described herein, e.g., a population of cells that include a cell described herein, e.g., a cell of any of the aforementioned cell aspects and embodiments. In aspects, the invention provides a population of cells, wherein at least about 50%, e.g., at least about 60%, e.g., at least about 70%, e.g., at least about 80%, e.g., at least about 90% (e.g., at least about 95%, at least about 96%, at least about 97%, at least about 98%, or at least about 99%) of the cells of the population are a cell described herein, e.g., a cell of any of the aforementioned cell aspects and embodiments. In aspects, the population of cells (e.g., a cell of the population of cells) is capable of differentiating into a population of differentiated cells, e.g., a population of cells of an erythroid lineage (e.g., a population of red blood cells), and wherein said population of differentiated cells has an increased percentage of F cells (e.g., at least about 15%, at least about 17%, at least about 20%, at least about 25%, at least about 30%, or at least about 40% higher percentage of F cells) e.g., relative to a population of unmodified cells of the same type. In aspects, the F cells of the population of differentiated cells produce an average of at least about 6 picograms (e.g., at least about 7 picograms, at least about 8 picograms, at least about 9 picograms, at least about 10 picograms, or from about 8 to about 9 picograms, or from about 9 to about 10 picograms) fetal hemoglobin per cell.

In an aspect, including in any of the aforementioned population of cell aspects and embodiments, the invention provides population of cells, including: 1) at least 1e6 CD34+ cells/kg body weight of the patient to whom the cells are to be administered; 2) at least 2e6 CD34+ cells/kg body weight of the patient to whom the cells are to be administered; 3) at least 3e6 CD34+ cells/kg body weight of the patient to whom the cells are to be administered; 4) at least 4e6 CD34+ cells/kg body weight of the patient to whom the cells are to be administered; or 5) from 2e6 to 10e6 CD34+ cells/kg body weight of the patient to whom the cells are to be administered. In embodiments, at least about 40%, e.g., at least about 50%, (e.g., at least about 60%, at least about 70%, at least about 80%, or at least about 90%) of the cells of the population are CD34+ cells. In embodiments, at least about 5%, e.g., at least about 10%, e.g., at least about 15%, e.g., at least about 20%, e.g., at least about 30% of the cells of the population are CD34+CD90+ cells. In embodiments, the population of cells is derived from umbilical cord blood, peripheral blood (e.g., mobilized peripheral blood), or bone marrow, e.g., is derived from bone marrow. In embodiments, the population of cells includes, e.g., consists of, mammalian cells, e.g., human cells. In embodiments, the population of cells is autologous relative to a patient to which it is to be administered. In other embodiments, the population of cells is allogeneic relative to a patient to which it is to be administered.

In an aspect, the invention provides a composition including a cell described herein, e.g., a cell of any of the aforementioned cell aspects and embodiments, or a population of cells described herein, e.g., a population of cells of any of the aforementioned population of cell aspects and embodiments. In an aspect, the composition includes a pharmaceutically acceptable medium, e.g., a pharmaceutically acceptable medium suitable for cryopreservation.

In an aspect, the invention provides a method of treating a hemoglobinopathy, including administering to a patient a cell described herein, e.g., a cell of any of the aforementioned cell aspects and embodiments, a population of cells described herein, e.g., a population of cells of any of the aforementioned population of cell aspects and embodiments, or a composition described herein, e.g., a composition of any of the aforementioned composition aspects and embodiments.

In an aspect, the invention provides a method of increasing fetal hemoglobin expression in a mammal, including administering to a patient a cell described herein, e.g., a cell of any of the aforementioned cell aspects and embodiments, a population of cells described herein, e.g., a population of cells of any of the aforementioned population of cell aspects and embodiments, or a composition described herein, e.g., a composition of any of the aforementioned composition aspects and embodiments. In aspects, the hemoglobinopathy is beta-thalassemia. In aspects, the hemoglobinopathy is sickle cell disease.

In an aspect, the invention provides a method of preparing a cell (e.g., a population of cells) including:
(a) providing a cell (e.g., a population of cells) (e.g., a HSPC (e.g., a population of HSPCs));
(b) culturing said cell (e.g., said population of cells) ex vivo in a cell culture medium including a stem cell expander; and
(c) introducing into said cell a first gRNA molecule, e.g., described herein, e.g., a first gRNA molecule of any of the aforementioned gRNA molecule aspects and embodiments; a nucleic acid molecule encoding a first gRNA molecule; a composition described herein, e.g., a composition of any of the aforementioned composition aspects and embodiments; or a vector described herein, e.g., a vector of any of the aforementioned aspects and embodiments. In aspects of the method, after said introducing of step (c), said cell (e.g., population of cells) is capable of differentiating into a differentiated cell (e.g., population of differentiated cells), e.g., a cell of an erythroid lineage (e.g., population of cells of an erythroid lineage), e.g., a red blood cell (e.g., a population of red blood cells), and wherein said differentiated cell (e.g., population of differentiated cells) produces increased fetal hemoglobin, e.g., relative to the same cell which has not been subjected to step (c). In aspects of the method, the stem cell expander is: a) (1r,4r)-N1-(2-benzyl-7-(2-methyl-2H-tetrazol-5-yl)-9H-pyrimido[4,5-b]indol-4-yl)cyclohexane-1,4-diamine; b) methyl 4-(3-piperidin-1-ylpropylamino)-9H-pyrimido[4,5-b]indole-7-carboxylate; c) 4-(2-(2-(benzo[b]thiophen-3-yl)-9-isopropyl-9H-purin-6-ylamino)ethyl)phenol; d) (S)-2-(6-(2-(1H-indol-3-yl)ethylamino)-2-(5-fluoropyridin-3-yl)-9H-purin-9-yl)propan-1-ol; or e) combinations thereof (e.g., a combination of (1r,4r)-N1-(2-benzyl-7-(2-methyl-2H-tetrazol-5-yl)-9H-pyrimido[4,5-b]indol-4-yl)cyclohexane-1,4-diamine and (S)-2-(6-(2-(1H-indol-3-yl)ethylamino)-2-(5-fluoropyridin-3-yl)-9H-purin-9-yl)propan-1-ol). In embodiments, the stem cell expander is (S)-2-(6-(2-(1H-indol-3-yl)ethylamino)-2-(5-fluoropyridin-3-yl)-9H-purin-9-yl)propan-1-ol. In aspects, the cell culture medium includes thrombopoietin (Tpo), Flt3 ligand (Flt-3L), and human stem cell factor (SCF). In aspects, the cell culture medium further includes human interleukin-6 (IL-6). In aspects, the cell culture medium includes thrombopoietin (Tpo), Flt3 ligand (Flt-3L), and human stem cell factor (SCF) each at a concentration ranging from about 10 ng/mL to about 1000 ng/mL, for example, each at a concentration of about 50 ng/mL, for example, each at a concentration of 50 ng/mL. In aspects, the cell culture medium includes human interleukin-6 (IL-6) at a concentration ranging from about 10 ng/mL to about 1000 ng/mL, for example, at a concentration of about 50 ng/mL, for example, at a concentration of 50 ng/mL. In aspects, the cell culture medium includes a stem cell expander at a concentration ranging from about 1 nM to about 1 mM, for example, at a concentration ranging from about 1 μM to about 100 nM, for example, at a concentration ranging from about 500 nM to about 750 nM. In aspects, the cell culture medium includes a stem cell expander at a concentration of about 500 nM, e.g., at a concentration of 500 nM. In aspects, the cell culture medium includes a stem cell expander at a concentration of about 750 nM, e.g., at a concentration of 750 nM.

In aspects of the method of preparing a cell (e.g., a population of cells), the culturing of step (b) includes a period of culturing before the introducing of step (c), for example, the period of culturing before the introducing of step (c) is at least 12 hours, e.g., is for a period of about 1 day to about 12 days, e.g., is for a period of about 1 day to about 6 days, e.g., is for a period of about 1 day to about 3 days, e.g., is for a period of about 1 day to about 2 days, e.g., is for a period of about 2 days. In aspects of the method of preparing a cell (e.g., a population of cells), including in any of the aforementioned aspects and embodiments of the method, the culturing of step (b) includes a period of culturing after the introducing of step (c), for example, the period of culturing after the introducing of step (c) is at least 12 hours, e.g., is for a period of about 1 day to about 12 days, e.g., is for a period of about 1 day to about 6 days, e.g., is for a period of about 2 days to about 4 days, e.g., is for a period of about 2 days or is for a period of about 3 days or is for a period of about 4 days. In aspects of the method of preparing a cell (e.g., a population of cells), including in any of the aforementioned aspects and embodiments of the method, the population of cells is expanded at least 4-fold, e.g., at least 5-fold, e.g., at least 10-fold, e.g., relative to cells which are not cultured according to step (b).

In aspects of the method of preparing a cell (e.g., a population of cells), including in any of the aforementioned aspects and embodiments of the method, the introducing of step (c) includes an electroporation. In aspects, the electroporation includes 1 to 5 pulses, e.g., 1 pulse, and wherein each pulse is at a pulse voltage ranging from 700 volts to 2000 volts and has a pulse duration ranging from 10 ms to 100 ms. In aspects, the electroporation includes, e.g., consists of, 1 pulse. In aspects, the pulse (or more than one pulse) voltage ranges from 1500 to 1900 volts, e.g., is 1700 volts. In aspects, the pulse duration of the one pulse or more than one pulse ranges from 10 ms to 40 ms, e.g., is 20 ms. In aspects of the method of preparing a cell (e.g., a population of cells), including in any of the aforementioned aspects and embodiments of the method, the cell (e.g., population of cells) provided in step (a) is a human cell (e.g., a population of human cells). In aspects of the method of preparing a cell (e.g., a population of cells), including in any of the aforementioned aspects and embodiments of the method, the cell (e.g., population of cells) provided in step (a) is isolated from bone marrow, peripheral blood (e.g., mobilized peripheral blood) or umbilical cord blood. In aspects of the method of preparing a cell (e.g., a population of cells), including in any of the aforementioned aspects and embodiments of the method, the cell (e.g., population of cells) provided in step (a) is isolated from bone marrow, e.g., is isolated from bone marrow of a patient suffering from a hemoglobinopathy.

In aspects of the method of preparing a cell (e.g., a population of cells), including in any of the aforementioned aspects and embodiments of the method, the population of cells provided in step (a) is enriched for CD34+ cells.

In aspects of the method of preparing a cell (e.g., a population of cells), including in any of the aforementioned aspects and embodiments of the method, subsequent to the introducing of step (c), the cell (e.g., population of cells) is cryopreserved.

In aspects of the method of preparing a cell (e.g., a population of cells), including in any of the aforementioned aspects and embodiments of the method, subsequent to the introducing of step (c), the cell (e.g., population of cells) includes: a) an indel at or near a genomic DNA sequence complementary to the targeting domain of the first gRNA molecule; or b) a deletion including sequence, e.g., substantially all the sequence, complementary to the targeting domain of the first gRNA molecule (e.g., at least 90% complementary to the gRNA targeting domain, e.g., fully complementary to the gRNA targeting domain) in the ZNF644 gene region.

In aspects of the method of preparing a cell (e.g., a population of cells), including in any of the aforementioned aspects and embodiments of the method, after the introducing of step (c), at least about 40%, at least about 50%, at least about 60%, at least about 70%, at least about 80%, at least about 90%, at least about 95%, at least about 96%, at least about 97%, at least about 98% or at least about 99% of the cells of the population of cells include an indel at or near a genomic DNA sequence complementary to the targeting domain of the first gRNA molecule.

In an aspect, the invention provides a cell (e.g., population of cells), obtainable by a method of preparing a cell (e.g., a population of cells) described herein, e.g., described in any of the aforementioned method of preparing a cell aspects and embodiments.

In an aspect, the invention provides a method of treating a hemoglobinopathy in a human patient, including administering to a human patient a composition including a cell described herein, e.g., a cell of any of the aforementioned cell aspects and embodiments; or a population of cells described herein, e.g., a population of cells of any of the aforementioned population of cell aspects and embodiments. In aspects, the hemoglobinopathy is beta-thalassemia. In aspects, the hemoglobinopathy is sickle cell disease.

In an aspect, the invention provides a method of increasing fetal hemoglobin expression in a human patient, including administering to said human patient a composition including a cell described herein, e.g., a cell of any of the aforementioned cell aspects and embodiments; or a population of cells described herein, e.g., a population of cells of any of the aforementioned population of cell aspects and embodiments. In aspects, the human patients has beta-thalassemia. In aspects, the human patient has sickle cell disease.

In aspects of the method of treating a hemoglobinopathy or the method of increasing fetal hemoglobin expression, the human patient is administered a composition including at least about 1e6 cells (e.g., cells as described herein) per kg body weight of the human patient, e.g., at least about 1e6 CD34+ cells (e.g., cells as described herein) per kg body weight of the human patient. In aspects of the method of treating a hemoglobinopathy or the method of increasing fetal hemoglobin expression, the human patient is administered a composition including at least about 2e6 cells (e.g., cells as described herein) per kg body weight of the human patient, e.g., at least about 2e6 CD34+ cells (e.g., cells as described herein) per kg body weight of the human patient. In aspects of the method of treating a hemoglobinopathy or the method of increasing fetal hemoglobin expression, the human patient is administered a composition including about 2e6 cells (e.g., cells as described herein) per kg body weight of the human patient, e.g., about 2e6 CD34+ cells (e.g., cells as described herein) per kg body weight of the human patient. In aspects of the method of treating a hemoglobinopathy or the method of increasing fetal hemoglobin expression, the human patient is administered a composition including at least about 3e6 cells (e.g., cells as described herein) per kg body weight of the human patient, e.g., at least about 3e6 CD34+ cells (e.g., cells as described herein) per kg body weight of the human patient. In aspects of the method of treating a hemoglobinopathy or the method of increasing fetal hemoglobin expression, the human patient is administered a composition including about 3e6 cells (e.g., cells as described herein) per kg body weight of the human patient, e.g., about 3e6 CD34+ cells (e.g., cells as described herein) per kg body weight of the human patient. In aspects of the method of treating a hemoglobinopathy or the method of increasing fetal hemoglobin expression, the human patient is administered a composition including from about 2e6 to about 10e6 cells (e.g., cells as described herein) per kg body weight of the human patient, e.g., from about 2e6 to about 10e6 CD34+ cells (e.g., cells as described herein) per kg body weight of the human patient.

Also provided herein are methods for treating a hemoglobinopathy and by administering to a patient a cell or population of cells or a composition containing such cell or population of cells described herein, or a composition that reduces ZNF644 gene expression and/or ZNF644 protein activity. In aspects, the composition that reduces ZNF644 gene expression and/or ZNF644 protein activity comprises a small molecule compound, siRNA, shRNA, antisense oligonucleotide (ASO), miRNA, anti-microRNA oligonucleotide (AMO) or any combination thereof. In aspects, the hemoglobinopathy is beta-thalassemia or sickle cell disease.

Also provided herein are methods for increasing fetal hemoglobin expression in a mammal by administering to a patient a cell or population of cells or a composition containing such cell or population of cells described herein, or a composition that reduces ZNF644 gene expression and/or ZNF644 protein activity. In aspects, the composition that reduces ZNF644 gene expression and/or ZNF644 protein activity comprises a small molecule compound, siRNA, shRNA, antisense oligonucleotide (ASO), miRNA, anti-microRNA oligonucleotide (AMO) or any combination thereof.

In an aspect, the invention provides: a gRNA molecule described herein, e.g., a gRNA molecule of any of the aforementioned gRNA molecule aspects and embodiments; a composition described herein, e.g., a composition of any of the aforementioned composition aspects and embodiments, a nucleic acid described herein, e.g., a nucleic acid of any of the aforementioned nucleic acid aspects and embodiments; a vector described herein, e.g., a vector of any of the aforementioned vector aspects and embodiments; a cell described herein, e.g., a cell of any of the aforementioned cell aspects and embodiments; or a population of cells described herein, e.g., a population of cells of any of the aforementioned population of cells aspects and embodiments, or a composition that reduces ZNF644 gene expression and/or ZNF644 protein activity aspects and embodiments, for use as a medicament. In aspects, the composition that reduces ZNF644 gene expression and/or ZNF644 protein activity comprises a small molecule compound, siRNA, shRNA, antisense oligonucleotide (ASO), miRNA, anti-microRNA oligonucleotide (AMO) or any combination thereof.

In an aspect, the invention provides: a gRNA molecule described herein, e.g., a gRNA molecule of any of the aforementioned gRNA molecule aspects and embodiments; a composition described herein, e.g., a composition of any of the aforementioned composition aspects and embodiments, a nucleic acid described herein, e.g., a nucleic acid of any of the aforementioned nucleic acid aspects and embodiments; a vector described herein, e.g., a vector of any of the aforementioned vector aspects and embodiments; a cell described herein, e.g., a cell of any of the aforementioned cell aspects and embodiments; or a population of cells described herein, e.g., a population of cells of any of the aforementioned population of cells aspects and embodiments, or a composition that reduces ZNF644 gene expression and/or ZNF644 protein activity aspects and embodiments, for use in the manufacture of a medicament. In aspects, the composition that reduces ZNF644 gene expression and/or ZNF644 protein activity comprises a small molecule compound, siRNA, shRNA, antisense oligonucleotide (ASO), miRNA, anti-microRNA oligonucleotide (AMO) or any combination thereof.

In an aspect, the invention provides: a gRNA molecule described herein, e.g., a gRNA molecule of any of the aforementioned gRNA molecule aspects and embodiments; a composition described herein, e.g., a composition of any of the aforementioned composition aspects and embodiments, a nucleic acid described herein, e.g., a nucleic acid of any of the aforementioned nucleic acid aspects and embodiments; a vector described herein, e.g., a vector of any of the aforementioned vector aspects and embodiments; a cell described herein, e.g., a cell of any of the aforementioned cell aspects and embodiments; or a population of cells described herein, e.g., a population of cells of any of the aforementioned population of cells aspects and embodiments, or a composition that reduces ZNF644 gene expression and/or ZNF644 protein activity aspects and embodiments, for use in the treatment of a disease. In aspects, the composition that reduces ZNF644 gene expression and/or ZNF644 protein activity comprises a small molecule compound, siRNA, shRNA, antisense oligonucleotide (ASO), miRNA, anti-microRNA oligonucleotide (AMO) or any combination thereof.

In an aspect, the invention provides: a gRNA molecule described herein, e.g., a gRNA molecule of any of the aforementioned gRNA molecule aspects and embodiments; a composition described herein, e.g., a composition of any of the aforementioned composition aspects and embodiments, a nucleic acid described herein, e.g., a nucleic acid of any of the aforementioned nucleic acid aspects and embodiments; a vector described herein, e.g., a vector of any of the aforementioned vector aspects and embodiments; a cell described herein, e.g., a cell of any of the aforementioned cell aspects and embodiments; or a population of cells described herein, e.g., a population of cells of any of the aforementioned population of cells aspects and embodiments, or a composition that reduces ZNF644 gene expression and/or ZNF644 protein activity aspects and embodiments, for use in the treatment of a disease, wherein the disease is a hemoglobinopathy, for example, beta-thalassemia or sickle cell disease. In aspects, the composition that reduces ZNF644 gene expression and/or ZNF644 protein activity comprises a small molecule compound, siRNA, shRNA, antisense oligonucleotide (ASO), miRNA, anti-microRNA oligonucleotide (AMO) or any combination thereof.

### BRIEF DESCRIPTION OF THE DRAWINGS

FIG. 1A Frequency of HbF+ cells due to shRNA-mediated loss of ZNF644 in human mobilized peripheral blood CD34+ derived erythroid cells.

FIG. 1B Frequency of HbF+ cells due to CRISPR/Cas9-mediated loss of ZNF644 in human mobilized peripheral blood CD34+ derived erythroid cells.

### DETAILED DESCRIPTION

### Abbreviations

BSA bovine serum albumin
Cas9 CRISPR associated protein 9
CRISPR Clustered regularly interspaced short palindromic repeats
crRNA CRISPR RNA
DMEM Dulbecco's modified eagle media
FACS fluorescence-activated cell sorting
FBS fetal bovine serum
FITC fluorescein
h or hr hour
HbF Fetal hemoglobin
HEPES (4-(2-hydroxyethyl)-1-piperazineethanesulfonic acid)
IMDM Iscove's modified Dulbecco's medium
KCl potassium chloride
LCMS liquid chromatography mass spectrometry
M molar
mL milliliter
mM millimolar
mPB mobilized peripheral blood
PBS phosphate buffered saline
rhEPO recombinant human erythropoietin
rhIL-3 recombinant human interleukin-3
rhIL-6 recombinant human interleukin-6
rhSCF recombinant human stem cell factor
rhTPO recombinant human thrombopoietin
RNP ribonucleoprotein
shRNA short hairpin RNA
ZNF644 Zinc Finger Protein 644

### Definitions

The terms “CRISPR system,” “Cas system” or “CRISPR/Cas system” refer to a set of molecules comprising an RNA-guided nuclease or other effector molecule and a gRNA molecule that together are necessary and sufficient to direct and effect modification of nucleic acid at a target sequence by the RNA-guided nuclease or other effector molecule. In one embodiment, a CRISPR system comprises a gRNA and a Cas protein, e.g., a Cas9 protein. Such systems comprising a Cas9 or modified Cas9 molecule are referred to herein as “Cas9 systems” or “CRISPR/Cas9 systems.” In one example, the gRNA molecule and Cas molecule may be complexed, to form a ribonuclear protein (RNP) complex.

The terms “guide RNA,” “guide RNA molecule,” “gRNA molecule” or “gRNA” are used interchangeably, and refer to a set of nucleic acid molecules that promote the specific directing of a RNA-guided nuclease or other effector molecule (typically in complex with the gRNA molecule) to a target sequence. In some embodiments, said directing is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In embodiments, a gRNA molecule consists of a single contiguous polynucleotide molecule, referred to herein as a “single guide RNA” or “sgRNA” and the like. In other embodiments, a gRNA molecule consists of a plurality, usually two, polynucleotide molecules, which are themselves capable of association, usually through hybridization, referred to herein as a “dual guide RNA” or “dgRNA,” and the like. gRNA molecules are described in more detail below, but generally include a targeting domain and a tracr. In embodiments the targeting domain and tracr are disposed on a single polynucleotide. In other embodiments, the targeting domain and tracr are disposed on separate polynucleotides.

The term “targeting domain” as the term is used in connection with a gRNA, is the portion of the gRNA molecule that recognizes, e.g., is complementary to, a target sequence, e.g., a target sequence within the nucleic acid of a cell, e.g., within a gene.

The term “crRNA” as the term is used in connection with a gRNA molecule, is a portion of the gRNA molecule that comprises a targeting domain and a region that interacts with a tracr to form a flagpole region.

The term “target sequence” refers to a sequence of nucleic acids complimentary, for example fully complementary, to a gRNA targeting domain. In embodiments, the target sequence is disposed on genomic DNA. In an embodiment the target sequence is adjacent to (either on the same strand or on the complementary strand of DNA) a protospacer adjacent motif (PAM) sequence recognized by a protein having nuclease or other effector activity, e.g., a PAM sequence recognized by Cas9. In embodiments, the target sequence is a target sequence within a gene or locus that affects expression of a globin gene, e.g., that affects expression of beta globin or fetal hemoglobin (HbF). In embodiments, the target sequence is a target sequence within ZNF644 gene region.

The term “flagpole” as used herein in connection with a gRNA molecule, refers to the portion of the gRNA where the crRNA and the tracr bind to, or hybridize to, one another.

The term “tracr” as used herein in connection with a gRNA molecule, refers to the portion of the gRNA that binds to a nuclease or other effector molecule. In embodiments, the tracr comprises nucleic acid sequence that binds specifically to Cas9. In embodiments, the tracr comprises nucleic acid sequence that forms part of the flagpole.

The terms “Cas9” or “Cas9 molecule” refer to an enzyme from bacterial Type II CRISPR/Cas system responsible for DNA cleavage. Cas9 also includes wild-type protein as well as functional and non-functional mutants thereof. In embodiments, the Cas9 is a Cas9 of S. pyogenes.

The term “complementary” as used in connection with nucleic acid, refers to the pairing of bases, A with T or U, and G with C. The term complementary refers to nucleic acid molecules that are completely complementary, that is, form A to T or U pairs and G to C pairs across the entire reference sequence, as well as molecules that are at least 80%, 85%, 90%, 95%, 99% complementary.

“Template Nucleic Acid” as used in connection with homology-directed repair or homologous recombination, refers to nucleic acid to be inserted at the site of modification by the CRISPR system donor sequence for gene repair (insertion) at site of cutting.

An “indel,” as the term is used herein, refers to a nucleic acid comprising one or more insertions of nucleotides, one or more deletions of nucleotides, or a combination of insertions and deletions of nucleotides, relative to a reference nucleic acid, that results after being exposed to a composition comprising a gRNA molecule, for example a CRISPR system. Indels can be determined by sequencing nucleic acid after being exposed to a composition comprising a gRNA molecule, for example, by NGS. With respect to the site of an indel, an indel is said to be “at or near” a reference site (e.g., a site complementary to a targeting domain of a gRNA molecule) if it comprises at least one insertion or deletion within about 10, 9, 8, 7, 6, 5, 4, 3, 2, or 1 nucleotide(s) of the reference site, or is overlapping with part or all of said reference site (e.g., comprises at least one insertion or deletion overlapping with, or within 10, 9, 8, 7, 6, 5, 4, 3, 2, or 1 nucleotides of a site complementary to the targeting domain of a gRNA molecule, e.g., a gRNA molecule described herein). In embodiments, the indel is a large deletion, for example, comprising more than about 1 kb, more than about 2 kb, more than about 3 kb, more than about 4 kb, more than about 5 kb, more than about 6 kb, or more than about 10 kb of nucleic acid. In embodiments, the 5′ end, the 3′ end, or both the 5′ and 3′ ends of the large deletion are disposed at or near a target sequence of a gRNA molecule described herein. In embodiments, the large deletion comprises about 4.9 kb of DNA disposed between a target sequence of a gRNA molecule, e.g., described herein, disposed within the ZNF644 gene region.

An “indel pattern,” as the term is used herein, refers to a set of indels that results after exposure to a composition comprising a gRNA molecule. In an embodiment, the indel pattern consists of the top three indels, by frequency of appearance. In an embodiment, the indel pattern consists of the top five indels, by frequency of appearance. In an embodiment, the indel pattern consists of the indels which are present at greater than about 1% frequency relative to all sequencing reads. In an embodiment, the indel pattern consists of the indels which are present at greater than about 5% frequency relative to all sequencing reads. In an embodiment, the indel pattern consists of the indels which are present at greater than about 10% frequency relative to total number of indel sequencing reads (i.e., those reads that do not consist of the unmodified reference nucleic acid sequence). In an embodiment, the indel pattern includes of any 3 of the top five most frequently observed indels. The indel pattern may be determined, for example, by methods described herein, e.g., by sequencing cells of a population of cells which were exposed to the gRNA molecule.

An “off-target indel,” as the term is used herein, refers to an indel at or near a site other than the target sequence of the targeting domain of the gRNA molecule. Such sites may comprise, for example, 1, 2, 3, 4, 5 or more mismatch nucleotides relative to the sequence of the targeting domain of the gRNA. In exemplary embodiments, such sites are detected using targeted sequencing of in silico predicted off-target sites, or by an insertional method known in the art. With respect to the gRNAs described herein, examples of off-target indels are indels formed at sequences outside of the ZNF644 gene region. In exemplary embodiments the off-target indel is formed in a sequence of a gene, e.g., within a coding sequence of a gene.

The term “a” and “an” refers to one or to more than one (i.e., to at least one) of the grammatical object of the article. By way of example, “an element” means one element or more than one element.

The term “and/or” means either “and” or “or” unless indicated otherwise.

The term “about” when referring to a measurable value such as an amount, a temporal duration, and the like, is meant to encompass variations of 20% or in some instances ±10%, or in some instances ±5%, or in some instances ±1%, or in some instances ±0.1% from the specified value, as such variations are appropriate to perform the disclosed methods.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be synthesized or can be derived from a biological sample, or might be macromolecule besides a polypeptide. Such a biological sample can include, but is not limited to a tissue sample, a cell or a fluid with other biological components.

The term “autologous” refers to any material derived from the same individual into whom it is later to be re-introduced.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

“Derived from” as that term is used herein, indicates a relationship between a first and a second molecule. It generally refers to structural similarity between the first molecule and a second molecule and does not connotate or include a process or source limitation on a first molecule that is derived from a second molecule.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some versions contain an intron(s).

The terms “effective amount” and “therapeutically effective amount” are used interchangeably herein, and refer to an amount of a compound, formulation, material, or composition, as described herein effective to achieve a particular biological result, for example, reduction or inhibition of an enzyme or a protein activity, or ameliorate symptoms, alleviate conditions, slow or delay disease progression, or prevent a disease, etc. In one embodiment, the term “a therapeutically effective amount” refers to the amount of the compound of the disclosure that, when administered to a subject, is effective to (1) at least partially alleviate, prevent and/or ameliorate a condition, or a disorder or a disease (i) mediated by ZNF644, or (ii) associated with ZNF644 activity, or (iii) characterized by activity (normal or abnormal) of ZNF644: (2) reduce or inhibit the activity of ZNF644; or (3) reduce or inhibit the expression level of ZNF644 gene and/or protein. In another embodiment, the term “a therapeutically effective amount” refers to the amount of the compound of the disclosure that, when administered to a cell, or a tissue, or a non-cellular biological material, or a medium, is effective to at least partially reducing or inhibiting the activity of ZNF644; or at least partially reducing or inhibiting the expression level of ZNF644 gene and/or protein.

As used herein, the term “inhibit”, “inhibition” or “inhibiting” refers to the reduction or suppression of a given condition, symptom, or disorder, or disease, or a significant decrease in the baseline activity of a biological activity or process, or a decrease in the baseline expression level of a gene and/or a protein of interest.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

As used herein “modulator” or “degrader”, means, for example, a compound of the disclosure, that effectively modulates, decreases, or reduces the levels of a specific protein (e.g., ZNF644). The amount of a specific protein (e.g., ZNF644) can be measured by comparing the amount of the specific protein (e.g., ZNF644) remaining after treatment with a compound of the disclosure as compared to the initial amount or level of the specific protein (e.g., ZNF644) present as measured prior to treatment with a compound of the disclosure.

As used herein “selective modulator”, “selective degrader”, or “selective compound” means, for example, a compound of the disclosure, that effectively modulates, decreases, or reduces the levels of a specific protein (e.g., ZNF644) to a greater extent than any other protein. A “selective modulator”, “selective degrader”, or “selective compound” canbe identified, for example, by comparing the ability of a compound to modulate, decrease, or reduce the levels of or to degrade a specific protein (e.g., ZNF644) to its ability to modulate, decrease, or reduce the levels of other proteins. In some embodiments, the selectivity can be identified by measuring the EC₅₀ or IC₅₀ of the compounds.

As used herein in connection with a messenger RNA (mRNA), a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” or “IVT RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000 (SEQ ID NO: 3118), preferably greater than 64, more preferably greater than 100, most preferably greater than 300 or 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a disorder, e.g., a hemoglobinopathy, or the amelioration of one or more symptoms (preferably, one or more discernible symptoms) of a disorder, e.g., a hemoglobinopathy, resulting from the administration of one or more therapies (e.g., one or more therapeutic agents such as a gRNA molecule, CRISPR system, or modified cell of the invention). In specific embodiments, the terms “treat”, “treatment” and “treating” refer to the amelioration of at least one measurable physical parameter of a hemoglobinopathy disorder, not discernible by the patient. In other embodiments the terms “treat”, “treatment” and “treating” refer to the inhibition of the progression of a disorder, either physically by, e.g., stabilization of a discernible symptom, physiologically by, e.g., stabilization of a physical parameter, or both. In other embodiments the terms “treat”, “treatment” and “treating” refer to the reduction or stabilization of a symptom of a hemoglobinopathy, e.g., sickle cell disease or beta-thalassemia.

As used herein, the term “prevent”, “preventing” or “prevention” of any disease or disorder refers to the prophylactic treatment of the disease or disorder; or delaying the onset or progression of the disease or disorder.

As used herein, “HbF-dependent disease or disorder” means any disease or disorder which is directly or indirectly affected by the modulation of HbF protein levels. Preferable examples of such disease or disorders are hemoglobinopathies, such as sickle cell disease or a thalassemia (e.g., beta-thalassemia).

As used herein, a subject is “in need of” a treatment if such subject would benefit biologically, medically or in quality of life from such treatment.

The term “signal transduction pathway” refers to the biochemical relationship between avariety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human). Preferably, the term “subject” refers to primates (e.g., humans, male or female), dogs, rabbits, guinea pigs, pigs, rats and mice. In certain embodiments, the subject is a primate. In yet other embodiments, the subject is a human.

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid and/or protein is transferred or introduced into the host cell. A “transfected” or “transformed” or “transduced” cell is one which has been transfected, transformed or transduced with exogenous nucleic acid and/or protein. The cell includes the primary subject cell and its progeny.

The term “specifically binds,” refers to a molecule recognizing and binding with a binding partner (e.g., a protein or nucleic acid) present in a sample, but which molecule does not substantially recognize or bind other molecules in the sample.

The term “bioequivalent” refers to an amount of an agent other than the reference compound, required to produce an effect equivalent to the effect produced by the reference dose or reference amount of the reference compound.

“Refractory” as used herein refers to a disease, e.g., a hemoglobinopathy, that does not respond to a treatment. In embodiments, a refractory hemoglobinopathy can be resistant to a treatment before or at the beginning of the treatment. In other embodiments, the refractory hemoglobinopathy can become resistant during a treatment. A refractory hemoglobinopathy is also called a resistant hemoglobinopathy.

“Relapsed” as used herein refers to the return of a disease (e.g., hemoglobinopathy) or the signs and symptoms of a disease such as a hemoglobinopathy after a period of improvement, e.g., after prior treatment of a therapy, e.g., hemoglobinopathy therapy.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The term “ZNF644” refers to Widely-Interspaced Zinc Finger-Containing Protein or variants or homologs thereof that maintain its transcriptional activity (e.g. within at least 50%, 80%, 90%, 95%, 96%, 97%, 98%, 99% or 100% activity compared to ZNF644), and the gene encoding said protein, together with all introns and exons as well as its regulatory regions such as promoters and enhancers. This gene encodes a zinc-finger protein. ZNF644 is also known as Zinc Finger Protein 803, ZNF803, Widely Interspaced Zinc Finger Motifs, ZNF644 Zinc Finger. The term encompasses all isoforms and splice variants of ZNF644. The human gene encoding ZNF644 is mapped to chromosomal location Chromosome 19: 15,419,980-15,449,951 (by Ensembl). The human and murine amino acid and nucleic acid sequences can be found in a public database, such as GenBank, UniProt and Swiss-Prot., and the genomic sequence of human ZNF644 can be found in GenBank at NC_000019.10. The ZNF644 gene refers to this genomic location, including all introns and exon. There are multiple known isotypes of ZNF644. In some embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ZNF644 protein. Exemplary ZNF644 transcript variants and their genomic coordinates are shown in Table 4.

TABLE 4
Genomic coordinates of ZNF644 transcripts variants.

| Variant | Exon_id | chrom | Start | End | Variant_exon_name | Strand |
|---|---|---|---|---|---|---|
| NCBI_tv1_NM_201269.3 | exon_6 | chr1 | 90915306 | 90916990 | NCBI_tv1_NM_201269.3_exon_6 | — |
| NCBI_tv1_NM_201269.3 | exon_5 | chr1 | 90918052 | 90918154 | NCBI_tv1_NM_201269.3_exon_5 | — |
| NCBI_tv1_NM_201269.3 | exon_4 | chr1 | 90937485 | 90938090 | NCBI_tv1_NM_201269.3_exon_4 | — |
| NCBI_tv1_NM_201269.3 | exon_3 | chr1 | 90938272 | 90941309 | NCBI_tv1_NM_201269.3_exon_3 | — |
| NCBI_tv1_NM_201269.3 | exon_2 | chr1 | 90982310 | 90982370 | NCBI_tv1_NM_201269.3_exon_2 | — |
| NCBI_tv1_NM_201269.3 | exon_1 | chr1 | 91021990 | 91022035 | NCBI_tv1_NM_201269.3_exon_1 | — |
| NCBI_tv2_NM_032186.5 | exon_4 | chr1 | 90915300 | 90916990 | NCBI_tv2_NM_032186.5_exon_4 | — |
| NCBI_tv2_NM_032186.5 | exon_3 | chr1 | 90918052 | 90918154 | NCBI_tv2_NM_032186.5_exon_3 | — |
| NCBI_tv2_NM_032186.5 | exon_2 | chr1 | 90982310 | 90982370 | NCBI_tv2_NM_032186.5_exon_2 | — |
| NCBI_tv2_NM_032186.5 | exon_1 | chr1 | 91021990 | 21022255 | NCBI_tv2_NM_032186.5_exon_1 | — |
| NCBI_tv3_NM_016620.4 | exon_4 | chr1 | 90915300 | 90916990 | NCBI_tv3_NM_016620.4_exon_4 | — |
| NCBI_tv3_NM_016620.4 | exon_3 | chr1 | 90918052 | 90918154 | NCBI_tv3_NM_016620.4_exon_3 | — |
| NCBI_tv3_NM_016620.4 | exon_2 | chr1 | 90982310 | 90982370 | NCBI_tv3_NM_016620.4_exon_2 | — |
| NCBI_tv3_NM_016620.4 | exon_1 | chr1 | 91021273 | 91021508 | NCBI_tv3_NM_016620.4_exon_1 | — |
| ZNF644-201_(Ensembl) | exon_6 | chr1 | 90915304 | 90916990 | ZNF644-201_(Ensembl)_exon_6 | — |
| ZNF644-201_(Ensembl) | exon_5 | chr1 | 90918052 | 90918154 | ZNF644-201_(Ensembl)_exon_5 | — |
| ZNF644-201_(Ensembl) | exon_4 | chr1 | 90937485 | 90938090 | ZNF644-201_(Ensembl)_exon_4 | — |
| ZNF644-201_(Ensembl) | exon_3 | chr1 | 90938272 | 90941309 | ZNF644-201_(Ensembl)_exon_3 | — |
| ZNF644-201_(Ensembl) | exon_2 | chr1 | 90982310 | 90982370 | ZNF644-201_(Ensembl)_exon_2 | — |
| ZNF644-201_(Ensembl) | exon_1 | chr1 | 91021990 | 91022035 | ZNF644-201_(Ensembl)_exon_1 | — |
| ZNF644-202_(Ensembl) | exon_4 | chr1 | 90915304 | 90916990 | ZNF644-202_(Ensembl)_exon_4 | — |
| ZNF644-202_(Ensembl) | exon_3 | chr1 | 90918052 | 90918154 | ZNF644-202_(Ensembl)_exon_3 | — |
| ZNF644-202_(Ensembl) | exon_2 | chr1 | 90982310 | 90982370 | ZNF644-202_(Ensembl)_exon_2 | — |
| ZNF644-202_(Ensembl) | exon_1 | chr1 | 91021273 | 91021457 | ZNF644-202_(Ensembl)_exon_1 | — |
| ZNF644-203_(Ensembl) | exon_4 | chr1 | 90916185 | 90916990 | ZNF644-203_(Ensembl)_exon_4 | — |
| ZNF644-203_(Ensembl) | exon_3 | chr1 | 90918052 | 90918154 | ZNF644-203_(Ensembl)_exon_3 | — |
| ZNF644-203_(Ensembl) | exon_2 | chr1 | 90982310 | 90982370 | ZNF644-203_(Ensembl)_exon_2 | — |
| ZNF644-203_(Ensembl) | exon_1 | chr1 | 91021990 | 91022272 | ZNF644-203_(Ensembl)_exon_1 | — |
| ZNF644-204_(Ensembl) | exon_6 | chr1 | 90915298 | 90916990 | ZNF644-204_(Ensembl)_exon_6 | — |
| ZNF644-204_(Ensembl) | exon_5 | chr1 | 90918052 | 90918154 | ZNF644-204_(Ensembl)_exon_5 | — |
| ZNF644-204_(Ensembl) | exon_4 | chr1 | 90937485 | 90938090 | ZNF644-204_(Ensembl)_exon_4 | — |
| ZNF644-204_(Ensembl) | exon_3 | chr1 | 90938272 | 90941309 | ZNF644-204_(Ensembl)_exon_3 | — |
| ZNF644-204_(Ensembl) | exon_2 | chr1 | 90982310 | 90982370 | ZNF644-204_(Ensembl)_exon_2 | — |
| ZNF644-204_(Ensembl) | exon_1 | chr1 | 91021273 | 91021473 | ZNF644-204_(Ensembl)_exon_1 | — |
| ZNF644-205_(Ensembl) | exon_5 | chr1 | 90916390 | 90916990 | ZNF644-205_(Ensembl)_exon_5 | — |
| ZNF644-205_(Ensembl) | exon_4 | chr1 | 90918052 | 90918154 | ZNF644-205_(Ensembl)_exon_4 | — |
| ZNF644-205_(Ensembl) | exon_3 | chr1 | 90973153 | 90973213 | ZNF644-205_(Ensembl)_exon_3 | — |
| ZNF644-205_(Ensembl) | exon_2 | chr1 | 90982310 | 90982370 | ZNF644-205_(Ensembl)_exon_2 | — |
| ZNF644-205_(Ensembl) | exon_1 | chr1 | 91021990 | 91022014 | ZNF644-205_(Ensembl)_exon_1 | — |
| ZNF644-206_(Ensembl) | exon_2 | chr1 | 90972744 | 90973213 | ZNF644-206_(Ensembl)_exon_2 | — |
| ZNF644-206_(Ensembl) | exon_1 | chr1 | 90982310 | 90982370 | ZNF644-206_(Ensembl)_exon_1 | — |
| ZNF644-207_(Ensembl) | exon_3 | chr1 | 90916490 | 90916990 | ZNF644-207_(Ensembl)_exon_3 | — |
| ZNF644-207_(Ensembl) | exon_2 | chr1 | 90918052 | 90918154 | ZNF644-207_(Ensembl)_exon_2 | — |
| ZNF644-207_(Ensembl) | exon_1 | chr1 | 90935408 | 90935528 | ZNF644-207_(Ensembl)_exon_1 | — |
| ZNF644-208_(Ensembl) | exon_2 | chr1 | 91020529 | 91021201 | ZNF644-208_(Ensembl)_exon_2 | — |
| ZNF644-208_(Ensembl) | exon_1 | chr1 | 91021689 | 91021725 | ZNF644-208_(Ensembl)_exon_1 | — |
| ZNF644-209_(Ensembl) | exon_2 | chr1 | 90916791 | 90916990 | ZNF644-209_(Ensembl)_exon_2 | — |
| ZNF644-209_(Ensembl) | exon_1 | chr1 | 90918052 | 90918391 | ZNF644-209_(Ensembl)_exon_1 | — |
| ZNF644-210_(Ensembl) | exon_2 | chr1 | 91020448 | 91021201 | ZNF644-210_(Ensembl)_exon_2 | — |
| ZNF644-210_(Ensembl) | exon_1 | chr1 | 91021990 | 91022055 | ZNF644-210_(Ensembl)_exon_1 | — |
| ZNF644-211_(Ensembl) | exon_3 | chr1 | 90940949 | 90941309 | ZNF644-211_(Ensembl)_exon_3 | — |
| ZNF644-211_(Ensembl) | exon_2 | chr1 | 90982310 | 90982370 | ZNF644-211_(Ensembl)_exon_2 | — |
| ZNF644-211_(Ensembl) | exon_1 | chr1 | 91021990 | 91022081 | ZNF644-211_(Ensembl)_exon_1 | — |
| ZNF644-212_(Ensembl) | exon_3 | chr1 | 90937883 | 90937937 | ZNF644-212_(Ensembl)_exon_3 | — |
| ZNF644-212_(Ensembl) | exon_2 | chr1 | 90937939 | 90938090 | ZNF644-212_(Ensembl)_exon_2 | — |
| ZNF644-212_(Ensembl) | exon_1 | chr1 | 90938272 | 90940120 | ZNF644-212_(Ensembl)_exon_1 | — |

In embodiments, exemplary ZNF644 transcript variants along with their nucleotide and protein sequences are shown below in Table 5.

TABLE 5
ZNF644 transcript variants compositions.

| | | | SEQ | |
|---|---|---|---|---|
| name_of_ZNF644_ | transcript_variant_ | | ID | Encoded |
| transcript_variant | composition | seq_name | NO | protein |
| NCBI_tv1: NM_201269.3 | exon_01_nc.5; | NCBI_RefSeq_NM_ | 2614 | UniProt |
| | exon_01_nc.6; | 201269.3_transcript_ | | Q9H582-1 |
| | exon_05_nc.1; | variant_1_mRNA | | (SEQ ID NO: |
| | exon_05_c.1/nc.2; | | | 2642) |
| | exon_05_c.2/nc.3; | | | |
| | exon_07_c.1/nc.1; | | | |
| | exon_07_c.1/; | | | |
| | exon_07_c.1/nc.2; | | | |
| | exon_07_c.2; exon_08_c; | | | |
| | exon_08_c; exon_08_c/nc; | | | |
| | exon_08_c; | | | |
| | exon_10_c.1/nc.2; | | | |
| | exon_11_c.1/nc.1; | | | |
| | exon_11_nc.2; | | | |
| | exon_11_nc.3; | | | |
| | exon_11_nc.4; | | | |
| | exon_11_nc.5; | | | |
| | exon_11_nc.6 | | | |
| NCBI_tv2: NM_032186.5 | exon_01_nc.2; | NCBI_RefSeq_NM_ | 2615 | UniProt |
| | exon_01_nc.3; | 032186.5_transcript_ | | Q9H582-3 |
| | exon_01_nc.4; | variant_2_mRNA | | (SEQ ID NO: |
| | exon_01_nc.5; | | | 2643) |
| | exon_01_nc.6; | | | |
| | exon_05_nc.1; | | | |
| | exon_05_c.1/nc.2; | | | |
| | exon_05_c.2/nc.3; | | | |
| | exon_10_c.1/nc.2; | | | |
| | exon_11_c.1/nc.1; | | | |
| | exon_11_nc.2; | | | |
| | exon_11_nc.3; | | | |
| | exon_11_nc.4; | | | |
| | exon_11_nc.5; | | | |
| | exon_11_nc.6; | | | |
| | exon_11_nc.7; | | | |
| | exon_11_nc.8 | | | |
| NCBI_tv3: NM_016620.4 | exon_02_nc; | NCBI_RefSeq_NM_ | 2616 | UniProt |
| | exon_03_nc.1; | 016620.4_transcript_ | | Q9H582-3 |
| | exon_03_nc.2; | variant_3_mRNA | | (SEQ ID NO: |
| | exon_03_nc.3; | | | 2643) |
| | exon_05_nc.1; | | | |
| | exon_05_c.1/nc.2; | | | |
| | exon_05_c.2/nc.3; | | | |
| | exon_10_c.1/nc.2; | | | |
| | exon_11_c.1/nc.1; | | | |
| | exon_11_nc.2; | | | |
| | exon_11_nc.3; | | | |
| | exon_11_nc.4; | | | |
| | exon_11_nc.5; | | | |
| | exon_11_nc.6; | | | |
| | exon_11_nc.7; | | | |
| | exon_11_nc.8 | | | |
| ZNF644_201 (Ensembl) | exon_01_nc.4; | Ensembl_ENST000003 | 2617 | UniProt |
| | exon_01_nc.5; | 37393.10_ZNF644- | | Q9H582-1 |
| | exon_01_nc.6; | 201_cdna_protein_ | | (SEQ ID NO: |
| | exon_05_nc.1; | coding | | 2642) |
| | exon_05_c.1/nc.2; | | | |
| | exon_05_c.2/nc.3; | | | |
| | exon_07_c.1/nc.1; | | | |
| | exon_07_c.1/; | | | |
| | exon_07_c.1/nc.2; | | | |
| | exon_07_c.2; exon_08_c; | | | |
| | exon_08_c; exon_08_c/nc; | | | |
| | exon_08_c; | | | |
| | exon_10_c.1/nc.2; | | | |
| | exon_11_c.1/nc.1; | | | |
| | exon_11_nc.2; | | | |
| | exon_11_nc.3; | | | |
| | exon_11_nc.4; | | | |
| | exon_11_nc.5; | | | |
| | exon_11_nc.6; | | | |
| | exon_11_nc.7 | | | |
| ZNF644_202 (Ensembl) | exon_03_nc.3; | Ensembl_ENST000003 | 2618 | UniProt |
| | exon_05_nc.1; | 47275.9_ZNF644- | | Q9H582-3 |
| | exon_05_c.1/nc.2; | 202_cdna_protein_ | | (SEQ ID NO: |
| | exon_05_c.2/nc.3; | coding | | 2643) |
| | exon_10_c.1/nc.2; | | | |
| | exon_11_c.1/nc.1; | | | |
| | exon_11_nc.2; | | | |
| | exon_11_nc.3; | | | |
| | exon_11_nc.4; | | | |
| | exon_11_nc.5; | | | |
| | exon_11_nc.6; | | | |
| | exon_11_nc.7 | | | |
| ZNF644_203 (Ensembl) | exon_01_nc.1; | Ensembl_ENST000003 | 2619 | UniProt |
| | exon_01_nc.2; | 61321.5_ZNF644- | | Q9H582-3 |
| | exon_01_nc.3; | 203_cdna_protein_ | | (SEQ ID NO: |
| | exon_01_nc.4; | coding | | 2643) |
| | exon_01_nc.5; | | | |
| | exon_01_nc.6; | | | |
| | exon_05_nc.1; | | | |
| | exon_05_c.1/nc.2; | | | |
| | exon_05_c.2/nc.3; | | | |
| | exon_10_c.1/nc.2; | | | |
| | exon_11_c.1/nc.1; | | | |
| | exon_11_nc.2; | | | |
| | exon_11_nc.3; | | | |
| | exon_11_nc.4; | | | |
| | exon_11_nc.5 | | | |
| ZNF644_204 (Ensembl) | exon_03_nc.2; | Ensembl_ENST000003 | 2620 | UniProt |
| | exon_03_nc.3; | 70440.5_ZNF644- | | Q9H582-1 |
| | exon_05_nc.1; | 204_cdna_protein_ | | (SEQ ID NO: |
| | exon_05_c.1/nc.2; | coding | | 2642) |
| | exon_05_c.2/nc.3; | | | |
| | exon_07_c.1/nc.1; | | | |
| | exon_07_c.1/; | | | |
| | exon_07_c.1/nc.2; | | | |
| | exon_07_c.2; exon_08_c; | | | |
| | exon_08_c; exon_08_c/nc; | | | |
| | exon_08_c; | | | |
| | exon_10_c.1/nc.2; | | | |
| | exon_11_c.1/nc.1; | | | |
| | exon_11_nc.2; | | | |
| | exon_11_nc.3; | | | |
| | exon_11_nc.4; | | | |
| | exon_11_nc.5; | | | |
| | exon_11_nc.6; | | | |
| | exon_11_nc.7; | | | |
| | exon_11_nc.8; | | | |
| | exon_11_nc.9 | | | |
| ZNF644_205 | exon_01_nc.6; | Ensembl_ENST000004 | 2621 | — |
| (Ensembl)_nc | exon_05_nc.1; | 67231.5_ZNF644- | | |
| | exon_05_c.1/nc.2; | 205_cdna_processed_ | | |
| | exon_05_c.2/nc.3; | transcript | | |
| | exon_06_nc.1; | | | |
| | exon_10_c.1/nc.2; | | | |
| | exon_11_c.1/nc.1; | | | |
| | exon_11_nc.2; | | | |
| | exon_11_nc.3; | | | |
| | exon_11_nc.4 | | | |
| ZNF644_206 | exon_05_nc.1; | Ensembl_ENST000004 | 2622 | — |
| (Ensembl)_nc | exon_05_c.1/nc.2; | 74405.1_ZNF644- | | |
| | exon_05_c.2/nc.3; | 206_cdna_processed_ | | |
| | exon_06_nc.1; | transcript | | |
| | exon_06_nc.2 | | | |
| ZNF644_207 | exon_09_nc; | Ensembl_ENST000004 | 2623 | — |
| (Ensembl)_nc | exon_10_c.1/nc.2; | 79798.1_ZNF644- | | |
| | exon_11_c.1/nc.1; | 207_cdna_processed_ | | |
| | exon_11_nc.2; | transcript | | |
| | exon_11_nc.3 | | | |
| ZNF644_208 | exon_02_nc; exon_04_nc.1 | Ensembl_ENST000004 | 2624 | — |
| (Ensembl)_nc | | 82467.1_ZNF644- | | |
| | | 208_cdna_processed_ | | |
| | | transcript | | |
| ZNF644_209 | exon_10_nc.1; | Ensembl_ENST000004 | 2625 | — |
| (Ensembl)_nc | exon_10_c.1/nc.2; | 82709.1_ZNF644- | | |
| | exon_11_c.1/nc.1; | 209_cdna_processed_ | | |
| | exon_11_nc.2 | transcript | | |
| ZNF644_210 | exon_01_nc.2; | Ensembl_ENST000004 | 2626 | — |
| (Ensembl)_nc | exon_01_nc.3; | 95966.1_ZNF644- | | |
| | exon_01_nc.4; | 210_cdna_processed_ | | |
| | exon_01_nc.5; | transcript | | |
| | exon_01_nc.6; | | | |
| | exon_04_nc.1; | | | |
| | exon_04_nc.2 | | | |
| ZNF644_211 | exon_01_nc.3; | Ensembl_ENST000004 | 2627 | — |
| (Ensembl)_nc | exon_01_nc.4; | 98303.5_ZNF644- | | |
| | exon_01_nc.5; | 211_cdna_processed_ | | |
| | exon_01_nc.6; | transcript | | |
| | exon_05_nc.1; | | | |
| | exon_05_c.1/nc.2; | | | |
| | exon_05_c.2/nc.3; | | | |
| | exon_07_c.1/nc.1 | | | |
| ZNF644_212 (Ensembl) | exon_07_c.1/nc.2; | Ensembl_ENST000006 | 2628 | UniProt |
| | exon_07_c.2; exon_08_c; | 21077.1_ZNF644- | | A0A087WZL9-1 |
| | exon_08_c/nc | 212_cdna_protein_ | | (SEQ ID NO: |
| | | coding | | 2644) |

Accordingly, isoforms of ZNF644 protein have the amino acid sequences of SEQ ID NO: 2642 (UniProt Q91H582-1), SEQ ID NO: 2643 (UniProt Q9H-582-3), or SEQ ID NO: 2644 (UniProt AOA087WZL9-1).

As used herein, a human ZNF644 protein also encompasses proteins that have over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with ZNF644 isoform disclosed herein, wherein such proteins still have at least one of the functions of ZNF644.

The term “complementary” as used in connection with nucleic acid, refers to the pairing of bases, A with T or U, and G with C. The term complementary refers to nucleic acid molecules that are completely complementary, that is, form A to T or U pairs and G to C pairs across the entire reference sequence, as well as molecules that are at least 80%, 85%, 90%, 95%, 99% complementary.

The terms “hematopoietic stem and progenitor cell” or “HSPC” are used interchangeably, and refer to a population of cells comprising both hematopoietic stem cells (“HSCs”) and hematopoietic progenitor cells (“HPCs”). Such cells are characterized, for example, as CD34+. In exemplary embodiments, HSPCs are isolated from bone marrow. In other exemplary embodiments, HSPCs are isolated from peripheral blood. In other exemplary embodiments, HSPCs are isolated from umbilical cord blood. In an embodiment, HSPCs are characterized as CD34+/CD38−/CD90+/CD45RA−. In embodiments, the HSPCs are characterized as CD34+/CD90+/CD49f+ cells. In embodiments, the HSPCs are characterized as CD34+ cells. In embodiments, the HSPC s are characterized as CD34+/CD90+ cells. In embodiments, the HSPCs are characterized as CD34+/CD90+/CD45RA− cells.

“Stem cell expander” as used herein refers to a compound which causes cells, e.g., HSPCs, HSCs and/or HPCs to proliferate, e.g., increase in number, at a faster rate relative to the same cell types absent said agent. In one exemplary aspect, the stem cell expander is an antagonist of the aryl hydrocarbon receptor pathway. Additional examples of stem cell expanders are provided below. In embodiments, the proliferation, e.g., increase in number, is accomplished ex vivo.

“Engraftment” or “engraft” refers to the incorporation of a cell or tissue, e.g., a population of HSPCs, into the body of a recipient, e.g., a mammal or human subject. In one example, engraftment includes the growth, expansion and/or differentiation of the engrafted cells in the recipient. In an example, engraftment of HSPCs includes the differentiation and growth of said HSPCs into erythroid cells within the body of the recipient.

The term “Hematopoietic progenitor cells” (HPCs) as used herein refers to primitive hematopoietic cells that have a limited capacity for self-renewal and the potential for multilineage differentiation (e.g., myeloid, lymphoid), mono-lineage differentiation (e.g., myeloid or lymphoid) or cell-type restricted differentiation (e.g., erythroid progenitor) depending on placement within the hematopoietic hierarchy (Doulatov et al., Cell Stem Cell 2012).

“Hematopoietic stem cells” (HSCs) as used herein refer to immature blood cells having the capacity to self-renew and to differentiate into more mature blood cells comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages). HSCs are interchangeably described as stem cells throughout the specification. It is known in the art that such cells may or may not include CD34+ cells. CD34+ cells are immature cells that express the CD34 cell surface marker. CD34+ cells are believed to include a subpopulation of cells with the stem cell properties defined above. It is well known in the art that HSCs are multipotent cells that can give rise to primitive progenitor cells (e.g., multipotent progenitor cells) and/or progenitor cells committed to specific hematopoietic lineages (e.g., lymphoid progenitor cells). The stem cells committed to specific hematopoietic lineages may be of T cell lineage, B cell lineage, dendritic cell lineage, Langerhans cell lineage and/or lymphoid tissue-specific macrophage cell lineage. In addition, HSCs also refer to long term HSC (LT-HSC) and short term HSC (ST-HSC). ST-HSCs are more active and more proliferative than LT-HSCs. However, LT-HSC have unlimited self-renewal (i.e., they survive throughout adulthood), whereas ST-HSC have limited self-renewal (i.e., they survive for only a limited period of time). Any of these HSCs can be used in any of the methods described herein. Optionally, ST-HSCs are useful because they are highly proliferative and thus, quickly increase the number of HSCs and their progeny. Hematopoietic stem cells are optionally obtained from blood products. A blood product includes a product obtained from the body or an organ of the body containing cells of hematopoietic origin. Such sources include un-fractionated bone marrow, umbilical cord, peripheral blood (e.g., mobilized peripheral blood, e.g., mobilized with a mobilization agent such as G-CSF or Plerixafor® (AMD3100), or a combination of G-CSF and Plerixafor® (AMD3100)), liver, thymus, lymph and spleen. All of the aforementioned crude or un-fractionated blood products can be enriched for cells having hematopoietic stem cell characteristics in ways known to those of skill in the art. In an embodiment, HSCs are characterized as CD34+/CD38−/CD90+/CD45RA−. In embodiments, the HSCs are characterized as CD34+/CD90+/CD49f+ cells. In embodiments, the HSCs are characterized as CD34+ cells. In embodiments, the HSCs are characterized as CD34+/CD90+ cells. In embodiments, the HSCs are characterized as CD34+/CD90+/CD45RA− cells.

“Expansion” or “Expand” in the context of cells refers to an increase in the number of a characteristic cell type, or cell types, from an initial cell population of cells, which may or may not be identical. The initial cells used for expansion may not be the same as the cells generated from expansion.

“Cell population” refers to eukaryotic mammalian, preferably human, cells isolated from biological sources, for example, blood product or tissues and derived from more than one cell.

“Enriched” when used in the context of cell population refers to a cell population selected based on the presence of one or more markers, for example, CD34+.

The term “CD34+ cells” refers to cells that express at their surface CD34 marker. CD34+ cells can be detected and counted using for example flow cytometry and fluorescently labeled anti-CD34 antibodies.

“Enriched in CD34+ cells” means that a cell population has been selected based on the presence of CD34 marker. Accordingly, the percentage of CD34+ cells in the cell population after selection method is higher than the percentage of CD34+ cells in the initial cell population before selecting step based on CD34 markers. For example, CD34+ cells may represent at least 50%, 60%, 70%, 80% or at least 90% of the cells in a cell population enriched in CD34+ cells.

The terms “F cell” and “F-cell” refer to cells, usually erythrocytes (e.g., red blood cells) which contain and/or produce (e.g., express) fetal hemoglobin. For example, an F-cell is a cell that contains or produces detectible levels of fetal hemoglobin. For example, an F-cell is a cell that contains or produces at least 5 picograms of fetal hemoglobin. In another example, an F-cell is a cell that contains or produces at least 6 picograms of fetal hemoglobin. In another example, an F-cell is a cell that contains or produces at least 7 picograms of fetal hemoglobin. In another example, an F-cell is a cell that contains or produces at least 8 picograms of fetal hemoglobin. In another example, an F-cell is a cell that contains or produces at least 9 picograms of fetal hemoglobin. In another example, an F-cell is a cell that contains or produces at least 10 picograms of fetal hemoglobin. Levels of fetal hemoglobin may be measured using an assay described herein or by other method known in the art, for example, flow cytometry using an anti-fetal hemoglobin detection reagent, high performance liquid chromatography, mass spectrometry, or enzyme-linked immunoabsorbent assay.

An “inhibitor” is a siRNA (e.g., shRNA, miRNA, snoRNA), gRNA, compound or small molecule that inhibits cellular function (e.g., replication) e.g., by binding, partially or totally blocking stimulation, decrease, prevent, or delay activation, or inactivate, desensitize, or down-regulate signal transduction, gene expression or enzymatic activity necessary for protein activity. A “ZNF644 inhibitor” refers to a substance that results in a detectably lower expression of ZNF644 gene or ZNF644 protein or lower activity level of ZNF644 proteins as compared to those levels without such substance. In some embodiments, a ZNF644 inhibitor is a small molecule compound (e.g., a small molecule compound that can target ZNF644 for degradation). In some embodiments, a ZNF644 inhibitor is an anti-ZNF644 shRNA. In some embodiments, a ZNF644 inhibitor is an anti-ZNF644 siRNA. In some embodiments, a ZNF644 inhibitor is an anti-ZNF644 ASO. In some embodiments, a ZNF644 inhibitor is an anti-ZNF644 AMO. In some embodiments, a ZNF644 inhibitor is an anti-ZNF644 antisense nucleic acid. In some embodiments, a ZNF644 inhibitor is a composition or a cell or a population of cells (that comprises gRNA molecules described herein) described herein.

An “antisense nucleic acid” as referred to herein is a nucleic acid (e.g. DNA or RNA molecule) that is complementary to at least a portion of a specific target nucleic acid (e.g. an mRNA translatable into a protein) and is capable of reducing transcription of the target nucleic acid (e.g. mRNA from DNA) or reducing the translation of the target nucleic acid (e.g. mRNA) or altering transcript splicing (e.g. single stranded morpholino oligo). See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid (e.g. target mRNA). In embodiments, the antisense nucleic acid hybridizes to the target nucleic acid sequence (e.g. mRNA) under stringent hybridization conditions. In embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone modified nucleotides.

In the cell, the antisense nucleic acids hybridize to the corresponding mRNA, forming a double-stranded molecule. The antisense nucleic acids interfere with the translation of the mRNA, since the cell will not translate an mRNA that is double-stranded. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or precursors.

An “siRNA” refers to a nucleic acid that forms a double stranded RNA, which double stranded RNA has the ability to reduce or inhibit expression of a gene or target gene when the siRNA is present (e.g. expressed) in the same cell as the gene or target gene. The siRNA is typically about 5 to about 100 nucleotides in length, more typically about 10 to about 50 nucleotides in length, more typically about 15 to about 30 nucleotides in length, most typically about 20-30 base nucleotides, or about 20-25 or about 24-29 nucleotides in length, e.g., 20, 21, 22, 23, 24, 25, 26, 27, 28, 29, or 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002).

Of the double stranded RNA of an siRNA, the strand that is at least partially complementary to at least a portion of a specific target nucleic acid (e.g. a target nucleic acid sequence), such as an mRNA molecule (e.g. a target mRNA molecule), is called the antisense (or guide strand; and the other strand is called sense (or passenger strand). The passenger strand is degraded and the guide strand is incorporated into the RNA-induced silencing complex (RISC).

A short hairpin RNA or small hairpin RNA (shRNA/Hairpin Vector) is an artificial RNA molecule with a tight hairpin turn that can be used to silence target gene expression via RNA interference (RNAi).

Antisense oligonucleotides (ASOs) are single strands of DNA or RNA that are complementary to a chosen sequence. In the case of antisense RNA they prevent protein translation of certain messenger RNA strands by binding to them, in a process called hybridization. Antisense oligonucleotides can be used to target a specific, complementary (coding or non-coding) RNA. If binding takes place this hybrid can be degraded by the enzyme RNase H.

Anti-miRNA Oligonucleotides (also known as AMOs) refer to synthetically designed molecules (e.g., oligonucleotides) that are used to neutralize microRNA (miRNA) function in cells for desired responses.

The term “miRNA” is used in accordance with its plain ordinary meaning and refers to a small non-coding RNA molecule capable of post-transcriptionally regulating gene expression. In one embodiment, a miRNA is a nucleic acid that has substantial or complete identity to a target gene. In embodiments, the miRNA inhibits gene expression by interacting with a complementary cellular mRNA thereby interfering with the expression of the complementary mRNA. Typically, the miRNA is at least about 15-50 nucleotides in length (e.g., each complementary sequence of the miRNA is 15-50 nucleotides in length, and the miRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

“Nucleic acid” refers to deoxyribonucleotides or ribonucleotides and polymers thereof in either single-, double- or multiple-stranded form, or complements thereof. The term “polynucleotide” or “oligonucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformicacid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Unless otherwise stated, all genome or chromosome coordinates are according to hg38.

The gRNA molecules, compositions and methods described herein relate to genome editing in eukaryotic cells using the CRISPR/Cas9 system. In particular, the gRNA molecules, compositions and methods described herein relate to regulation of globin levels and are useful, for example, in regulating expression and production of globin genes and protein. The gRNA molecules, compositions and methods can be useful in the treatment of hemoglobinopathies.

I. gRNA Molecules

A gRNA molecule may have a number of domains, as described more fully below, however, a gRNA molecule typically comprises at least a crRNA domain (comprising a targeting domain) and a tracr. The gRNA molecules of the invention, used as a component of a CRISPR system, are useful for modifying (e.g., modifying the sequence) DNA at or near a target site. Such modifications include deletions and or insertions that result in, for example, reduced or eliminated expression of a functional product of the gene comprising the target site. These uses, and additional uses, are described more fully below.

In an embodiment, a unimolecular, or sgRNA comprises, preferably from 5′ to 3′: a crRNA (which contains a targeting domain complementary to a target sequence and a region that forms part of a flagpole (i.e., a crRNA flagpole region)); a loop; and a tracr (which contains a domain complementary to the crRNA flagpole region, and a domain which additionally binds a nuclease or other effector molecule, e.g., a Cas molecule, e.g., aCas9 molecule), and may take the following format (from 5′ to 3′):
[targeting domain]-[crRNA flagpole region]-[optional first flagpole extension]-[loop]-[optional first tracr extension]-[tracr flagpole region]-[tracr nuclease binding domain].

In embodiments, the tracr nuclease binding domain binds to a Cas protein, e.g., a Cas9 protein. In an embodiment, a bimolecular, or dgRNA comprises two polynucleotides; the first, preferably from 5′ to 3′: a crRNA (which contains a targeting domain complementary to a target sequence and a region that forms part of a flagpole; and the second, preferably from 5′ to 3′: a tracr (which contains a domain complementary to the crRNA flagpole region, and a domain which additionally binds a nuclease or other effector molecule, e.g., a Cas molecule, e.g., Cas9 molecule), and may take the following format (from 5′ to 3′):
Polynucleotide 1 (crRNA): [targeting domain]-[crRNA flagpole region]-[optional first flagpole extension]-[optional second flagpole extension]
Polynucleotide 2 (tracr): [optional first tracr extension]-[tracr flagpole region]-[tracr nuclease binding domain]

In embodiments, the tracr nuclease binding domain binds to a Cas protein, e.g., a Cas9 protein.

In some aspects, the targeting domain comprises or consists of a targeting domain sequence described herein, e.g., a targeting domain described in Table 1-Table 3, or a targeting domain comprising or consisting of 17, 18, 19, or 20 (preferably 20) consecutive nucleotides of a targeting domain sequence described in Table 1-Table 3.

In some aspects, the flagpole, e.g., the crRNA flagpole region, comprises, from 5′ to 3′: GUUUUAGAGCUA (SEQ ID NO: 3110).

In some aspects, the flagpole, e.g., the crRNA flagpole region, comprises, from 5′ to 3′: GUUUAAGAGCUA (SEQ ID NO: 3111).

In some aspects the loop comprises, from 5′ to 3′: GAAA (SEQ ID NO: 3114).

In some aspects the tracr comprises, from 5′ to 3′: UAGCAAGUUAAAAUAAGGCUAGUCCGUUAUCAACUUGAAAAAGUGGCACCGAGUCGG UGC (SEQ ID NO: 3115) and is preferably used in a gRNA molecule comprising SEQ ID NO: 3110.

In some aspects the tracr comprises, from 5′ to 3′: UAGCAAGUUUAAAUAAGGCUAGUCCGUUAUCAACUUGAAAAAGUGGCACCGAGUCGG UGC (SEQ ID NO: 3116) and is preferably used in a gRNA molecule comprising SEQ ID NO: 3111.

In some aspects, the gRNA may also comprise, at the 3′ end, additional U nucleic acids. For example the gRNA may comprise an additional 1, 2, 3, 4, 5, 6, 7, 8, 9, or 10 U nucleic acids (SEQ ID NO: 3177) at the 3′ end. In an embodiment, the gRNA comprises an additional 4 U nucleic acids at the 3′ end. In the case of dgRNA, one or more of the polynucleotides of the dgRNA (e.g., the polynucleotide comprising the targeting domain and the polynucleotide comprising the tracr) may comprise, at the 3′ end, additional U nucleic acids. For example, the case of dgRNA, one or more of the polynucleotides of the dgRNA (e.g., the polynucleotide comprising the targeting domain and the polynucleotide comprising the tracr) may comprise an additional 1, 2, 3, 4, 5, 6, 7, 8, 9, or 10 U nucleic acids (SEQ ID NO: 3177) at the 3′ end. In an embodiment, in the case of dgRNA, one or more of the polynucleotides of the dgRNA (e.g., the polynucleotide comprising the targeting domain and the polynucleotide comprising the tracr) comprises an additional 4 U nucleic acids at the 3′ end. In an embodiment of a dgRNA, only the polynucleotide comprising the tracr comprises the additional U nucleic acid(s), e.g., 4 U nucleic acids. In an embodiment of a dgRNA, only the polynucleotide comprising the targeting domain comprises the additional U nucleic acid(s). In an embodiment of a dgRNA, both the polynucleotide comprising the targeting domain and the polynucleotide comprising the tracr comprise the additional U nucleic acids, e.g., 4 U nucleic acids.

In some aspects, the gRNA may also comprise, at the 3′ end, additional A nucleic acids. For example the gRNA may comprise an additional 1, 2, 3, 4, 5, 6, 7, 8, 9, or 10 A nucleic acids (SEQ ID NO: 3178) at the 3′ end. In an embodiment, the gRNA comprises an additional 4 A nucleic acids at the 3′ end. In the case of dgRNA, one or more of the polynucleotides of the dgRNA (e.g., the polynucleotide comprising the targeting domain and the polynucleotide comprising the tracr) may comprise, at the 3′ end, additional A nucleic acids. For example, the case of dgRNA, one or more of the polynucleotides of the dgRNA (e.g., the polynucleotide comprising the targeting domain and the polynucleotide comprising the tracr) may comprise an additional 1, 2, 3, 4, 5, 6, 7, 8, 9, or 10 A nucleic acids (SEQ ID NO: 3178) at the 3′ end. In an embodiment, in the case of dgRNA, one or more of the polynucleotides of the dgRNA (e.g., the polynucleotide comprising the targeting domain and the polynucleotide comprising the tracr) comprises an additional 4 A nucleic acids at the 3′ end. In an embodiment of a dgRNA, only the polynucleotide comprising the tracr comprises the additional A nucleic acid(s), e.g., 4 A nucleic acids. In an embodiment of a dgRNA, only the polynucleotide comprising the targeting domain comprises the additional A nucleic acid(s). In an embodiment of a dgRNA, both the polynucleotide comprising the targeting domain and the polynucleotide comprising the tracr comprise the additional U nucleic acids, e.g., 4 A nucleic acids.

In embodiments, one or more of the polynucleotides of the gRNA molecule may comprise a cap at the 5′ end.

In an embodiment, a unimolecular, or sgRNA comprises, preferably from 5′ to 3′: a crRNA (which contains a targeting domain complementary to a target sequence; a crRNA flagpole region; first flagpole extension; a loop; a first tracr extension (which contains a domain complementary to at least a portion of the first flagpole extension); and a tracr (which contains a domain complementary to the crRNA flagpole region, and a domain which additionally binds a Cas9 molecule). In some aspects, the targeting domain comprises a targeting domain sequence described herein, e.g., a targeting domain described in Table 1-Table 3, or a targeting domain comprising or consisting of 17, 18, 19, or 20 (preferably 20) consecutive nucleotides of a targeting domain sequence described in Table 1-Table 3, for example the 3′ 17, 18, 19, or 20 (preferably 20) consecutive nucleotides of a targeting domain sequence described in Table 1-Table 3.

In aspects comprising a first flagpole extension and/or a first tracr extension, the flagpole, loop and tracr sequences may be as described above. In general any first flagpole extension and first tracr extension may be employed, provided that they are complementary. In embodiments, the first flagpole extension and first tracr extension consist of 3, 4, 5, 6, 7, 8, 9, 10 or more complementary nucleotides.

In some aspects, the first flagpole extension comprises, from 5′ to 3′: UGCUG (SEQ ID NO: 3112). In some aspects, the first flagpole extension consists of SEQ ID NO: 3112.

In some aspects, the first tracr extension comprises, from 5′ to 3′: CAGCA (SEQ ID NO: 3117). In some aspects, the first tracr extension consists of SEQ ID NO: 3117.

In an embodiment, a dgRNA comprises two nucleic acid molecules. In some aspects, the dgRNA comprises a first nucleic acid which contains, preferably from 5′ to 3′: a targeting domain complementary to a target sequence; a crRNA flagpole region; optionally a first flagpole extension; and, optionally, a second flagpole extension; and a second nucleic acid (which may be referred to herein as a tracr), and comprises at least a domain which binds a Cas molecule, e.g., a Cas9 molecule) comprising preferably from 5′ to 3′: optionally a first tracr extension; and a tracr (which contains a domain complementary to the crRNA flagpole region, and a domain which additionally binds a Cas, e.g., Cas9, molecule). The second nucleic acid may additionally comprise, at the 3′ end (e.g., 3′ to the tracr) additional U nucleic acids. For example the tracr may comprise an additional 1, 2, 3, 4, 5, 6, 7, 8, 9, or 10 U nucleic acids (SEQ ID NO: 3177) at the 3′ end (e.g., 3′ to the tracr). The second nucleic acid may additionally or alternately comprise, at the 3′ end (e.g., 3′ to the tracr) additional A nucleic acids. For example the tracr may comprise an additional 1, 2, 3, 4, 5, 6, 7, 8, 9, or 10 A nucleic acids (SEQ ID NO: 3178) at the 3′ end (e.g., 3′ to the tracr). In some aspects, the targeting domain comprises a targeting domain sequence described herein, e.g., a targeting domain described in Table 1-Table 3, or a targeting domain comprising or consisting of 17, 18, 19, or 20 (preferably 20) consecutive nucleotides of a targeting domain sequence described in Table 1-Table 3.

In aspects involving a dgRNA, the crRNA flagpole region, optional first flagpole extension, optional first tracr extension and tracr sequences may be as described above.

In some aspects, the optional second flagpole extension comprises, from 5′ to 3′: UUUUG (SEQ ID NO: 3113).

In embodiments, the 3′ 1, 2, 3, 4, or 5 nucleotides, the 5′ 1, 2, 3, 4, or 5 nucleotides, or both the 3′ and 5′ 1, 2, 3, 4, or 5 nucleotides of the gRNA molecule (and in the case of a dgRNA molecule, the polynucleotide comprising the targeting domain and/or the polynucleotide comprising the tracr) are modified nucleic acids, as described more fully in section XIII, below.

The domains are discussed briefly below:

### 1) The Targeting Domain:

Guidance on the selection of targeting domains can be found, e.g., in Fu Y et al. NAT BIOTECHNOL 2014 (doi: 10.1038/nbt.2808) and Sternberg S H et al. NATURE 2014 (doi: 10.1038/nature13011).

The targeting domain comprises a nucleotide sequence that is complementary, e.g., at least 80, 85, 90, 95, or 99% complementary, e.g., fully complementary, to the target sequence on the target nucleic acid. The targeting domain is part of an RNA molecule and will therefore comprise the base uracil (U), while any DNA encoding the gRNA molecule will comprise the base thymine (T). While not wishing to be bound by theory, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence.

In an embodiment, the targeting domain is 5 to 50, e.g., 10 to 40, e.g., 10 to 30, e.g., 15 to 30, e.g., 15 to 25 nucleotides in length. In an embodiment, the targeting domain is 15, 16, 17, 18, 19, 20, 21, 22, 23, 24 or 25 nucleotides in length. In an embodiment, the targeting domain is 16 nucleotides in length. In an embodiment, the targeting domain is 17 nucleotides in length. In an embodiment, the targeting domain is 18 nucleotides in length. In an embodiment, the targeting domain is 19 nucleotides in length. In an embodiment, the targeting domain is 20 nucleotides in length. In embodiments, the aforementioned 16, 17, 18, 19, or 20 nucleotides comprise the 5′-16, 17, 18, 19, or 20 nucleotides from a targeting domain described in Table 1-Table 3. In embodiments, the aforementioned 16, 17, 18, 19, or 20 nucleotides comprise the 3′-16, 17, 18, 19, or 20 nucleotides from a targeting domain described in Table 1-Table 3.

Without being bound by theory, it is believed that the 8, 9, 10, 11 or 12 nucleic acids of the targeting domain disposed at the 3′ end of the targeting domain is important for targeting the target sequence, and may thus be referred to as the “core” region of the targeting domain. In an embodiment, the core domain is fully complementary with the target sequence.

The strand of the target nucleic acid with which the targeting domain is complementary is referred to herein as the target sequence. In some aspects, the target sequence is disposed on a chromosome, e.g., is a target within a gene. In some aspects the target sequence is disposed within an exon of a gene. In some aspects the target sequence is disposed within an intron of a gene. In some aspects, the target sequence comprises, or is proximal (e.g., within 10, 20, 30, 40, 50, 100, 200, 300, 400, 500, or 1000 nucleic acids) to a binding site of a regulatory element, e.g., a promoter or transcription factor binding site, of a gene of interest. Some or all of the nucleotides of the domain can have a modification, e.g., modification found in Section XIII herein.

2) crRNA Flagpole Region:

The flagpole contains portions from both the crRNA and the tracr. The crRNA flagpole region is complementary with a portion of the tracr, and in an embodiment, has sufficient complementarity to a portion of the tracr to form a duplexed region under at least some physiological conditions, for example, normal physiological conditions. In an embodiment, the crRNA flagpole region is 5 to 30 nucleotides in length. In an embodiment, the crRNA flagpole region is 5 to 25 nucleotides in length. The crRNA flagpole region can share homology with, or be derived from, a naturally occurring portion of the repeat sequence from a bacterial CRISPR array. In an embodiment, it has at least 50% homology with a crRNA flagpole region disclosed herein, e.g., an S. pyogenes, or S. thermophilus, crRNA flagpole region.

In an embodiment, the flagpole, e.g., the crRNA flagpole region, comprises SEQ ID NO: 3110. In an embodiment, the flagpole, e.g., the crRNA flagpole region, comprises sequence having at least 50%, 60%, 70%, 80%, 85%, 90%, 95% or 99% homology with SEQ ID NO: 3110. In an embodiment, the flagpole, e.g., the crRNA flagpole region, comprises at least 5, 6, 7, 8, 9, 10, or 11 nucleotides of SEQ ID NO: 3110. In an embodiment, the flagpole, e.g., the crRNA flagpole region, comprises SEQ ID NO: 3111. In an embodiment, the flagpole comprises sequence having at least 50%, 60%, 70%, 80%, 85%, 90%, 95% or 99% homology with SEQ ID NO: 3111. In an embodiment, the flagpole, e.g., the crRNA flagpole region, comprises at least 5, 6, 7, 8, 9, 10, or 11 nucleotides of SEQ ID NO: 3111.

Some or all of the nucleotides of the domain can have a modification, e.g., modification described in Section XIII herein.

### 3) First Flagpole Extension

When a tracr comprising a first tracr extension is used, the crRNA may comprise a first flagpole extension. In general any first flagpole extension and first tracr extension may be employed, provided that they are complementary. In embodiments, the first flagpole extension and first tracr extension consist of 3, 4, 5, 6, 7, 8, 9, 10 or more complementary nucleotides.

The first flagpole extension may comprise nucleotides that are complementary, e.g., 80%, 85%, 90%, 95% or 99%, e.g., fully complementary, with nucleotides of the first tracr extension. In some aspects, the first flagpole extension nucleotides that hybridize with complementary nucleotides of the first tracr extension are contiguous. In some aspects, the first flagpole extension nucleotides that hybridize with complementary nucleotides of the first tracr extension are discontinuous, e.g., comprises two or more regions of hybridization separated by nucleotides that do not base pair with nucleotides of the first tracr extension. In some aspects, the first flagpole extension comprises at least 2, 3, 4, 5, 6, 7, 8, 9, 10, 11, 12, 13, 14, 15, 16, 17, 18, 19, 20 or more nucleotides. In some aspects, the first flagpole extension comprises, from 5′ to 3′: UGCUG (SEQ ID NO: 3112). In some aspects, the first flagpole extension consists of SEQ ID NO: 3112. In some aspects the first flagpole extension comprises nucleic acid that is at least 80%, 85%, 90%, 95% or 99% homology to SEQ ID NO: 3112.

Some or all of the nucleotides of the first tracr extension can have a modification, e.g., modification found in Section XIII herein.

### 4) The Loop

A loop serves to link the crRNA flagpole region (or optionally the first flagpole extension, when present) with the tracr (or optionally the first tracr extension, when present) of a sgRNA. The loop can link the crRNA flagpole region and tracr covalently or non-covalently. In an embodiment, the linkage is covalent. In an embodiment, the loop covalently couples the crRNA flagpole region and tracr. In an embodiment, the loop covalently couples the first flagpole extension and the first tracr extension. In an embodiment, the loop is, or comprises, a covalent bond interposed between the crRNA flagpole region and the domain of the tracr which hybridizes to the crRNA flagpole region. Typically, the loop comprises one or more, e.g., 2, 3, 4, 5, 6, 7, 8, 9, or 10 nucleotides.

In dgRNA molecules the two molecules can be associated by virtue of the hybridization between at least a portion of the crRNA (e.g., the crRNA flagpole region) and at least a portion of the tracr (e.g., the domain of the tracr which is complementary to the crRNA flagpole region).

A wide variety of loops are suitable for use in sgRNAs. Loops can consist of a covalent bond, or be as short as one or a few nucleotides, e.g., 1, 2, 3, 4, or 5 nucleotides in length. In an embodiment, a loop is 2, 3, 4, 5, 6, 7, 8, 9, 10, 15, 20, or 25 or more nucleotides in length. In an embodiment, a loop is 2 to 50, 2 to 40, 2 to 30, 2 to 20, 2 to 10, or 2 to 5 nucleotides in length. In an embodiment, a loop shares homology with, or is derived from, a naturally occurring sequence. In an embodiment, the loop has at least 50% homology with a loop disclosed herein. In an embodiment, the loop comprises SEQ ID NO: 3114.

Some or all of the nucleotides of the domain can have a modification, e.g., modification described in Section XIII herein.

### 5) The Second Flagpole Extension

In an embodiment, a dgRNA can comprise additional sequence, 3′ to the crRNA flagpole region or, when present, the first flagpole extension, referred to herein as the second flagpole extension. In an embodiment, the second flagpole extension is, 2-10, 2-9, 2-8, 2-7, 2-6, 2-5, or 2-4 nucleotides in length. In an embodiment, the second flagpole extension is 2, 3, 4, 5, 6, 7, 8, 9, or 10 or more nucleotides in length. In an embodiment, the second flagpole extension comprises SEQ ID NO: 3113.

### 6) The Tracr:

The tracr is the nucleic acid sequence required for nuclease, e.g., Cas9, binding. Without being bound by theory, it is believed that each Cas9 species is associated with a particular tracr sequence. Tracr sequences are utilized in both sgRNA and in dgRNA systems. In an embodiment, the tracr comprises sequence from, or derived from, an S. pyogenes tracr. In some aspects, the tracr has a portion that hybridizes to the flagpole portion of the crRNA, e.g., has sufficient complementarity to the crRNA flagpole region to form a duplexed region under at least some physiological conditions (sometimes referred to herein as the tracr flagpole region or a tracr domain complementary to the crRNA flagpole region). In embodiments, the domain of the tracr that hybridizes with the crRNA flagpole region comprises at least 5, 6, 7, 8, 9, 10, 11, 12, 13, 14, 15, 16, 17, 18, 19, or 20 nucleotides that hybridize with complementary nucleotides of the crRNA flagpole region. In some aspects, the tracr nucleotides that hybridize with complementary nucleotides of the crRNA flagpole region are contiguous. In some aspects, the tracr nucleotides that hybridize with complementary nucleotides of the crRNA flagpole region are discontinuous, e.g., comprises two or more regions of hybridization separated by nucleotides that do not base pair with nucleotides of the crRNA flagpole region. In some aspects, the portion of the tracr that hybridizes to the crRNA flagpole region comprises, from 5′ to 3′: UAGCAAGUUAAAA (SEQ ID NO: 3119). In some aspects, the portion of the tracr that hybridizes to the crRNA flagpole region comprises, from 5′ to 3′: UAGCAAGUUUAAA (SEQ ID NO: 3120). In embodiments, the sequence that hybridizes with the crRNA flagpole region is disposed on the tracr 5′- to the sequence of the tracr that additionally binds a nuclease, e.g., a Cas molecule, e.g., a Cas9 molecule.

The tracr further comprises a domain that additionally binds to a nuclease, e.g., a Cas molecule, e.g., a Cas9 molecule. Without being bound by theory, it is believed that Cas9 from different species bind to different tracr sequences. In some aspects, the tracr comprises sequence that binds to a S. pyogenes Cas9 molecule. In some aspects, the tracr comprises sequence that binds to a Cas9 molecule disclosed herein. In some aspects, the domain that additionally binds a Cas9 molecule comprises, from 5′ to 3′: UAAGGCUAGUCCGUUAUCAACUUGAAAAAGUGGCACCGAGUCGGUGC (SEQ ID NO: 3121). In some aspects the domain that additionally binds a Cas9 molecule comprises, from 5′ to 3′: UAAGGCUAGUCCGUUAUCAACUUGAAAAAGUGGCACCGAGUCGGUGCUUUU (SEQ ID NO: 3122).

In some embodiments, the tracr comprises SEQ ID NO: 3115. In some embodiments, the tracr comprises SEQ ID NO: 3116.

Some or all of the nucleotides of the tracr can have a modification, e.g., modification found in Section XIII herein. In embodiments, the gRNA (e.g., the sgRNA or the tracr and/or crRNA of a dgRNA), e.g., any of the gRNA or gRNA components described above, comprises an inverted abasic residue at the 5′ end, the 3′ end or both the 5′ and 3′ end of the gRNA. In embodiments, the gRNA (e.g., the sgRNA or the tracr and/or crRNA of a dgRNA), e.g., any of the gRNA or gRNA components described above, comprises one or more phosphorothioate bonds between residues at the 5′ end of the polynucleotide, for example, a phosphrothioate bond between the first two 5′ residues, between each of the first three 5′ residues, between each of the first four 5′ residues, or between each of the first five 5′ residues. In embodiments, the gRNA or gRNA component may alternatively or additionally comprise one or more phosphorothioate bonds between residues at the 3′ end of the polynucleotide, for example, a phosphrothioate bond between the first two 3′ residues, between each of the first three 3′ residues, between each of the first four 3′ residues, orbetween each of the first five 3′ residues. In an embodiment, the gRNA (e.g., the sgRNA or the tracr and/or crRNA of a dgRNA), e.g., any of the gRNA or gRNA components described above, comprises a phosphorothioate bond between each of the first four 5′ residues (e.g., comprises, e.g., consists of, three phosphorothioate bonds at the 5′ end(s)), and a phosphorothioate bond between each of the first four 3′ residues (e.g., comprises, e.g., consists of, three phosphorothioate bonds at the 3′ end(s)). In an embodiment, any of the phosphorothioate modifications described above are combined with an inverted abasic residue at the 5′ end, the 3′ end, or both the 5′ and 3′ ends of the polynucleotide. In such embodiments, the inverted abasic nucleotide may be linked to the 5′ and/or 3′ nucleotide by a phosphate bond or a phosphorothioate bond. In embodiments, the gRNA (e.g., the sgRNA or the tracr and/or crRNA of a dgRNA), e.g., any of the gRNA or gRNA components described above, comprises one or more nucleotides that include a 2′ O-methyl modification. In embodiments, each of the first 1, 2, 3, or more of the 5′ residues comprise a 2′ O-methyl modification. In embodiments, each of the first 1, 2, 3, or more of the 3′ residues comprise a 2′ O-methyl modification. In embodiments, the 4^{th}-to-terminal, 3^{rd}-to-terminal, and 2nd-to-terminal 3′ residues comprise a 2′ O-methyl modification. In embodiments, each of the first 1, 2, 3 or more of the 5′ residues comprise a 2′ O-methyl modification, and each of the first 1, 2, 3 or more of the 3′ residues comprise a 2′ O-methyl modification. In an embodiment, each of the first 3 of the 5′ residues comprise a 2′ O-methyl modification, and each of the first 3 of the 3′ residues comprise a 2′ O-methyl modification. In embodiments, each of the first 3 of the 5′ residues comprise a 2′ O-methyl modification, and the 4^{th}-to-terminal, 3^{d}-to-terminal, and 2nd-to-terminal 3′ residues comprise a 2′ O-methyl modification. In embodiments, any of the 2′ O-methyl modifications, e.g., as described above, may be combined with one or more phosphorothioate modifications, e.g., as described above, and/or one or more inverted abasic modifications, e.g., as described above. In an embodiment, the gRNA (e.g., the sgRNA or the tracr and/or crRNA of a dgRNA), e.g., any of the gRNA or gRNA components described above, comprises, e.g., consists of, a phosphorothioate bond between each of the first four 5′ residues (e.g., comprises, e.g., consists of three phosphorothioate bonds at the 5′ end of the polynucleotide(s)), a phosphorothioate bond between each of the first four 3′ residues (e.g., comprises, e.g., consists of three phosphorothioate bonds at the 5′ end of the polynucleotide(s)), a 2′ O-methyl modification at each of the first three 5′ residues, and a 2′ O-methyl modification at each of the first three 3′ residues. In an embodiment, the gRNA (e.g., the sgRNA or the tracr and/or crRNA of a dgRNA), e.g., any of the gRNA or gRNA components described above, comprises, e.g., consists of, a phosphorothioate bond between each of the first four 5′ residues (e.g., comprises, e.g., consists of three phosphorothioate bonds at the 5′ end of the polynucleotide(s)), a phosphorothioate bond between each of the first four 3′ residues (e.g., comprises, e.g., consists of three phosphorothioate bonds at the 5′ end of the polynucleotide(s)), a 2′ O-methyl modification at each of the first three 5′ residues, and a 2′ O-methyl modification at each of the 4^{th}-to-terminal, 3^{rd}-to-terminal, and 2nd-to-terminal 3′ residues.

In an embodiment, the gRNA (e.g., the sgRNA or the tracr and/or crRNA of a dgRNA), e.g., any of the gRNA or gRNA components described above, comprises, e.g., consists of, a phosphorothioate bond between each of the first four 5′ residues (e.g., comprises, e.g., consists of three phosphorothioate bonds at the 5′ end of the polynucleotide(s)), a phosphorothioate bond between each of the first four 3′ residues (e.g., comprises, e.g., consists of three phosphorothioate bonds at the 5′ end of the polynucleotide(s)), a 2′ O-methyl modification at each of the first three 5′ residues, a 2′ O-methyl modification at each of the first three 3′ residues, and an additional inverted abasic residue at each of the 5′ and 3′ ends.

In an embodiment, the gRNA (e.g., the sgRNA or the tracr and/or crRNA of a dgRNA), e.g., any of the gRNA or gRNA components described above, comprises, e.g., consists of, a phosphorothioate bond between each of the first four 5′ residues (e.g., comprises, e.g., consists of three phosphorothioate bonds at the 5′ end of the polynucleotide(s)), a phosphorothioate bond between each of the first four 3′ residues (e.g., comprises, e.g., consists of three phosphorothioate bonds at the 5′ end of the polynucleotide(s)), a 2′ O-methyl modification at each of the first three 5′ residues, and a 2′ O-methyl modification at each of the 4^{th}-to-terminal, 3^{r}d-to-terminal, and 2nd-to-terminal 3′ residues, and an additional inverted abasic residue at each of the 5′ and 3′ ends.

In an embodiment, the gRNA is a dgRNA and comprises, e.g., consists of:
crRNA:
mN*mN*mN*NNNNNNNNNNNNNNNNNGUUUUAGAGCUAU*mG*mC*mU (SEQ ID NO: 3179), where m indicates a base with 2′O-Methyl modification, * indicates a phosphorothioate bond, and N′s indicate the residues of the targeting domain, e.g., as described herein, (optionally with an inverted abasic residue at the 5′ and/or 3′ terminus); and
tracr:
AACAGCAUAGCAAGUUAAAAUAAGGCUAGUCCGUUAUCAACUUGAAAAAGUGGCACC GAGUCGGUGCUUUUUUU (SEQ ID NO: 3152) (optionally with an inverted abasic residue at the 5′ and/or 3′ tenninus).

In an embodiment, the gRNA is a dgRNA and comprises, e.g., consists of:
crRNA:
mN*mN*mN*NNNNNNNNNNNNNNNNNGUUUUAGAGCUAU*mG*mC*mU (SEQ ID NO: 3179), where m indicates a base with 2′O-Methyl modification, * indicates a phosphorothioate bond, and N′s indicate the residues of the targeting domain, e.g., as described herein, (optionally with an inverted abasic residue at the 5′ and/or 3′ terminus); and
tracr:
mA*mA*mC*AGCAUAGCAAGUUAAAAUAAGGCUAGUCCGUUAUCAACUUGAAAAAGUG GCACCGAGUCGGUGCUUUU*mU*mU*mU (SEQ ID NO: 3174), where m indicates a base with 2′O-Methyl modification, * indicates a phosphorothioate bond, and N′s indicate the residues of the targeting domain, e.g., as described herein, (optionally with an inverted abasic residue at the 5′ and/or 3′ terminus).

In an embodiment, the gRNA is a dgRNA and comprises, e.g., consists of:
crRNA:
mN*mN*mN*NNNNNNNNNNNNNNNNNGUUUUAGAGCUAUGCUGUU*mU*mU*mG (SEQ ID NO: 3180), where m indicates a base with 2′O-Methyl modification, * indicates a phosphorothioate bond, and N′s indicate the residues of the targeting domain, e.g., as described herein, (optionally with an inverted abasic residue at the 5′ and/or 3′ terminus); and
tracr:
AACAGCAUAGCAAGUUAAAAUAAGGCUAGUCCGUUAUCAACUUGAAAAAGUGGCACC GAGUCGGUGCUUUUUUU (SEQ ID NO: 3152) (optionally with an inverted abasic residue at the 5′ and/or 3′ tenninus).

In an embodiment, the gRNA is a dgRNA and comprises, e.g., consists of:
crRNA:
mN*mN*mN*NNNNNNNNNNNNNNNNNGUUUUAGAGCUAUGCUGUU*mU*mU*mG (SEQ ID NO: 3180), where m indicates a base with 2′O-Methyl modification, * indicates a phosphorothioate bond, and N′s indicate the residues of the targeting domain, e.g., as described herein, (optionally with an inverted abasic residue at the 5′ and/or 3′ terminus); and
tracr:
mA*mA*mC*AGCAUAGCAAGUUAAAAUAAGGCUAGUCCGUUAUCAACUUGAAAAAGUG GCACCGAGUCGGUGCUUUU*mU*mU*mU (SEQ ID NO: 3174), where m indicates a base with 2′O-Methyl modification, and * indicates a phosphorothioate bond (optionally with an inverted abasic residue at the 5′ and/or 3′ terminus).

In an embodiment, the gRNA is a dgRNA and comprises, e.g., consists of:
crRNA:

(SEQ ID NO: 3181)
NNNNNNNNNNNNNNNNNNNNGUUUUAGAGCUAUGCUGUUUUG,

where N′s indicate the residues of the targeting domain, e.g., as described herein, (optionally with an inverted abasic residue at the 5′ and/or 3′ terminus); and
tracr:
mA*mA*mC*AGCAUAGCAAGUUAAAAUAAGGCUAGUCCGUUAUCAACUUGAAAAAGUG GCACCGAGUCGGUGCUUUU*mU*mU*mU (SEQ ID NO: 3174), where m indicates a base with 2′O-Methyl modification, and * indicates a phosphorothioate bond (optionally with an inverted abasic residue at the 5′ and/or 3′ terminus).

In an embodiment, the gRNA is a sgRNA and comprises, e.g., consists of:
NNNNNNNNNNNNNNNNNNNNGUUUUAGAGCUAGAAAUAGCAAGUUAAAAUAAGGCUA GUCCGUUAUCAACUUGAAAAAGUGGCACCGAGUCGGUGCUUUU (SEQ ID NO: 3182), where m indicates a base with 2′O-Methyl modification, * indicates a phosphorothioate bond, and N′s indicate the residues of the targeting domain, e.g., as described herein, (optionally with an inverted abasic residue at the 5′ and/or 3′ terminus).

In an embodiment, the gRNA is a sgRNA and comprises, e.g., consists of:
mN*mN*mN*NNNNNNNNNNNNNNNNNGUUUUAGAGCUAGAAAUAGCAAGUUAAAAUA AGGCUAGUCCGUUAUCAACUUGAAAAAGUGGCACCGAGUCGGUGCU*mU*mU*mU (SEQ ID NO: 3183), where m indicates a base with 2′O-Methyl modification, * indicates a phosphorothioate bond, and N′s indicate the residues of the targeting domain, e.g., as described herein, (optionally with an inverted abasic residue at the 5′ and/or 3′ terminus).

In an embodiment, the gRNA is a sgRNA and comprises, e.g., consists of:
mN*mN*mN*NNNNNNNNNNNNNNNNNGUUUUAGAGCUAGAAAUAGCAAGUUAAAAUA AGGCUAGUCCGUUAUCAACUUGAAAAAGUGGCACCGAGUCGGUGCmU*mU*mU*U (SEQ ID NO: 3184), where m indicates a base with 2′O-Methyl modification, * indicates a phosphorothioate bond, and N′s indicate the residues of the targeting domain, e.g., as described herein, (optionally with an inverted abasic residue at the 5′ and/or 3′ terminus).

### 7) First Tracr Extension

Where the gRNA comprises a first flagpole extension, the tracr may comprise a first tracr extension. The first tracr extension may comprise nucleotides that are complementary, e.g., 80%, 85%, 90%, 95% or 99%, e.g., fully complementary, with nucleotides of the first flagpole extension. In some aspects, the first tracr extension nucleotides that hybridize with complementary nucleotides of the first flagpole extension are contiguous. In some aspects, the first tracr extension nucleotides that hybridize with complementary nucleotides of the first flagpole extension are discontinuous, e.g., comprises two or more regions of hybridization separated by nucleotides that do not base pair with nucleotides of the first flagpole extension. In some aspects, the first tracr extension comprises at least 2, 3, 4, 5, 6, 7, 8, 9, 10, 11, 12, 13, 14, 15, 16, 17, 18, 19, 20 or more nucleotides. In some aspects, the first tracr extension comprises SEQ ID NO: 3117. In some aspects the first tracr extension comprises nucleic acid that is at least 80%, 85%, 90%, 95% or 99% homology to SEQ ID NO: 3117.

Some or all of the nucleotides of the first tracr extension can have a modification, e.g., modification found in Section XIII herein.

In some embodiments, the sgRNA may comprise, from 5′ to 3′, disposed 3′ to the targeting domain:

a)
(SEQ ID NO: 3123)
GUUUUAGAGCUAGAAAUAGCAAGUUAAAAUAAGGCUAGUCCGUUA
UCAACUUGAAAAAGUGGCACCGAGUCGGUGC;
b)
(SEQ ID NO: 3124)
GUUUAAGAGCUAGAAAUAGCAAGUUUAAAUAAGGCUAGUCCGUUA
UCAACUUGAAAAAGUGGCACCGAGUCGGUGC;
c)
(SEQ ID NO: 3125)
GUUUUAGAGCUAUGCUGGAAACAGCAUAGCAAGUUAAAAUAAGGC
UAGUCCGUUAUCAACUUGAAAAAGUGGCACCGAGUCGGUGC;
d)
(SEQ ID NO: 3126)
GUUUAAGAGCUAUGCUGGAAACAGCAUAGCAAGUUUAAAUAAGGC
UAGUCCGUUAUCAACUUGAAAAAGUGGCACCGAGUCGGUGC;

e) any of a) to d), above, further comprising, at the 3′ end, at least 1, 2, 3, 4, 5, 6 or 7 uracil (U) nucleotides, e.g., 1, 2, 3, 4, 5, 6, or 7 uracil (U) nucleotides;
f) any of a) to d), above, further comprising, at the 3′ end, at least 1, 2, 3, 4, 5, 6 or 7 adenine (A) nucleotides, e.g., 1, 2, 3, 4, 5, 6, or 7 adenine (A) nucleotides; or
g) any of a) to f), above, further comprising, at the 5′ end (e.g., at the 5′ terminus, e.g., 5′ to the targeting domain), at least 1, 2, 3, 4, 5, 6 or 7 adenine (A) nucleotides, e.g., 1, 2, 3, 4, 5, 6, or 7 adenine (A) nucleotides. In embodiments, any of a) to g) above is disposed directly 3′ to the targeting domain. In an embodiment, a sgRNA of the invention comprises, e.g., consists of, from 5′ to 3′: [targeting domain]—

(SEQ ID NO: 3159)
GUUUUAGAGCUAGAAAUAGCAAGUUAAAAUAAGGCUAGUC
CGUUAUCAACUUGAAAAAGUGGCACCGAGUCGGUGCUUUU.

In an embodiment, a sgRNA of the invention comprises, e.g., consists of, from 5′ to 3′: [targeting domain]—

(SEQ ID NO: 3155)
GUUUAAGAGCUAUGCUGGAAACAGCAUAGCAAGUUUAAAUA
AGGCUAGUCCGUUAUCAACUUGAAAAAGUGGCACCGAGUCG
GUGCUUUU.

In some embodiments, the dgRNA may comprise:
A crRNA comprising, from 5′ to 3′, preferably disposed directly 3′ to the targeting domain:

a)
(SEQ ID NO: 3110)
GUUUUAGAGCUA;
b)
(SEQ ID NO: 3111)
GUUUAAGAGCUA;
c)
(SEQ ID NO: 3127)
GUUUUAGAGCUAUGCUG;
d)
(SEQ ID NO: 3128)
GUUUAAGAGCUAUGCUG;
e)
(SEQ ID NO: 3129)
GUUUUAGAGCUAUGCUGUUUUG;
f)
(SEQ ID NO: 3130)
GUUUAAGAGCUAUGCUGUUUUG;
or
g)
(SEQ ID NO: 3154)
GUUUUAGAGCUAUGCU:

and a tracr comprising, from 5′ to 3′:

a)
(SEQ ID NO: 3115)
UAGCAAGUUAAAAUAAGGCUAGUCCGUUAUCAACUUGAAAAAGUG
GCACCGAGUCGGUGC;
b)
(SEQ ID NO: 3116)
UAGCAAGUUUAAAUAAGGCUAGUCCGUUAUCAACUUGAAAAAGUG
GCACCGAGUCGGUGC;
c)
(SEQ ID NO: 3131)
CAGCAUAGCAAGUUAAAAUAAGGCUAGUCCGUUAUCAACUUGAAA
AAGUGGCACCGAGUCGGUGC;
d)
(SEQ ID NO: 3132)
CAGCAUAGCAAGUUUAAAUAAGGCUAGUCCGUUAUCAACUUGAAAA
AGUGGCACCGAGUCGGUGC;
e)
(SEQ ID NO: 3152)
AACAGCAUAGCAAGUUAAAAUAAGGCUAGUCCGUUAUCAACUUGA
AAAAGUGGCACCGAGUCGGUGCUUUUUUU;
f)
(SEQ ID NO: 3153)
AACAGCAUAGCAAGUUUAAAUAAGGCUAGUCCGUUAUCAACUUGA
AAAAGUGGCACCGAGUCGGUGCUUUUUUU;
g)
(SEQ ID NO: 3160)
AACAGCAUAGCAAGUUAAAAUAAGGCUAGUCCGUUAUCAACUUGA
AAAAGUGGCACCGAGUCGGUGC
h)
(SEQ ID NO: 3155)
GUUUAAGAGCUAUGCUGGAAACAGCAUAGCAAGUUUAAAUAAGGC
UAGUCCGUUAUCAACUUGAAAAAGUGGCACCGAGUCGGUGCUUUU;
i)
(SEQ ID NO: 3156)
AGCAUAGCAAGUUAAAAUAAGGCUAGUCCGUUAUCAACUUGAAAA
AGUGGCACCGAGUCGGUGCUUU;
j)
(SEQ ID NO: 3157)
GUUGGAACCAUUCAAAACAGCAUAGCAAGUUAAAAUAAGGCUAGU
CCGUUAUCAACUUGAAAAAGUGGCACCGAGUCGGUGCUUU;

k) any of a) to j), above, further comprising, at the 3′ end, at least 1, 2, 3, 4, 5, 6 or 7 uracil (U) nucleotides, e.g., 1, 2, 3, 4, 5, 6, or 7 uracil (U) nucleotides;
l) any of a) to j), above, further comprising, at the 3′ end, at least 1, 2, 3, 4, 5, 6 or 7 adenine (A) nucleotides, e.g., 1, 2, 3, 4, 5, 6, or 7 adenine (A) nucleotides; or
m) any of a) to 1), above, further comprising, at the 5′ end (e.g., at the 5′ terminus), at least 1, 2, 3, 4, 5, 6 or 7 adenine (A) nucleotides, e.g., 1, 2, 3, 4, 5, 6, or 7 adenine (A) nucleotides.

In an embodiment, the sequence of k), above comprises the 3′ sequence UUUUUU, e.g., if a U6 promoter is used for transcription. In an embodiment, the sequence of k), above, comprises the 3′ sequence UUUU, e.g., if an HI promoter is used for transcription. In an embodiment, sequence of k), above, comprises variable numbers of 3′ U's depending, e.g., on the termination signal of the pol-III promoter used. In an embodiment, the sequence of k), above, comprises variable 3′ sequence derived from the DNA template if a T7 promoter is used. In an embodiment, the sequence of k), above, comprises variable 3′ sequence derived from the DNA template, e.g., if in vitro transcription is used to generate the RNA molecule. In an embodiment, the sequence of k), above, comprises variable 3′ sequence derived from the DNA template, e.g., if a pol-II promoter is used to drive transcription. In an embodiment, the crRNA comprises, e.g., consists of, a targeting domain and, disposed 3′ to the targeting domain (e.g., disposed directly 3′ to the targeting domain), a sequence comprising, e.g., consisting of, SEQ ID NO: 3129, and the tracr comprises, e.g., consists of

(SEQ ID NO: 3152)
AACAGCAUAGCAAGUUAAAAUAAGGCUAGUCCGUUAU
CAACUUGAAAAAGUGGCACCGAGUCGGUGCUUUUUUU

In an embodiment, the crRNA comprises, e.g., consists of, a targeting domain and, disposed 3′ to the targeting domain (e.g., disposed directly 3′ to the targeting domain), a sequence comprising, e.g., consisting of, SEQ ID NO: 3130, and the tracr comprises, e.g., consists of,

(SEQ ID NO: 3153)
AACAGCAUAGCAAGUUUAAAUAAGGCUAGUCCGUUAU
CAACUUGAAAAAGUGGCACCGAGUCGGUGCUUUUUUU

In an embodiment, the crRNA comprises, e.g., consists of, a targeting domain and, disposed 3′ to the targeting domain (e.g., disposed directly 3′ to the targeting domain), a sequence comprising, e.g., consisting of, GUUUUAGAGCUAUGCU (SEQ ID NO: 3154), and the tracr comprises, e.g., consists of,

(SEQ ID NO: 3155)
GUUUAAGAGCUAUGCUGGAAACAGCAUAGCAAGUUUA
AAUAAGGCUAGUCCGUUAUCAACUUGAAAAAGUGGCA
CCGAGUCGGUGCUUUU

In an embodiment, the crRNA comprises, e.g., consists of, a targeting domain and, disposed 3′ to the targeting domain (e.g., disposed directly 3′ to the targeting domain), a sequence comprising, e.g., consisting of, GUUUUAGAGCUAUGCU (SEQ ID NO: 3154), and the tracr comprises, e.g., consists of, AGCAUAGCAAGUUAAAAUAAGGCUAGUCCGUUAUCAACUUGAAAAAGUGGCACCGAG UCGGUGCUUU (SEQ ID NO: 3156).

(SEQ ID NO: 3156)
AGCAUAGCAAGUUAAAAUAAGGCUAGUCCGUUAUCAA
CUUGAAAAAGUGGCACCGAGUCGGUGCUUU

In an embodiment, the crRNA comprises, e.g., consists of, a targeting domain and, disposed 3′ to the targeting domain (e.g., disposed directly 3′ to the targeting domain), a sequence comprising, e.g., consisting of, GUUUUAGAGCUAUGCUGUUUUG (SEQ ID NO: 3129), and the tracr comprises, e.g., consists of,

(SEQ ID NO: 3157)
GUUGGAACCAUUCAAAACAGCAUAGCAAGUUAAAAUA
AGGCUAGUCCGUUAUCAACUUGAAAAAGUGGCACCGA
GUCGGUGCUUU

### II. gRNA Targeting Domains Directed to ZNF644 Gene

Provided in the Table 1-Table 3 (at the end of the document) are targeting domains directed to ZNF644 gene regions, for gRNA molecules of the present invention, and for use in the various aspects of the present invention, for example, in altering expression of globin genes, for example, a fetal hemoglobin gene or a hemoglobin beta gene.

### III. Methods for Designing gRNAs

Methods for designing gRNAs are described herein, including methods for selecting, designing and validating target sequences. Exemplary targeting domains are also provided herein. Targeting Domains discussed herein can be incorporated into the gRNAs described herein.

Methods for selection and validation of target sequences as well as off-target analyses are described, e.g., in. Mali et al., 2013 SCIENCE 339(6121): 823-826; Hsu et al, 2013 NAT BIOTECHNOL, 31 (9): 827-32; Fu et al, 2014 NAT BIOTECHNOL, doi: 10.1038/nbt.2808. PubMed PM ID: 24463574; Heigwer et al, 2014 NAT METHODS 11 (2): 122-3. doi: 10.1038/nmeth.2812. PubMed PMID: 24481216; Bae et al, 2014 BIOINFORMATICS PubMed PMID: 24463181; Xiao A el al, 2014 BIOINFORMATICS PubMed PMID: 24389662.

For example, a software tool can be used to optimize the choice of gRNA within a user's target sequence, e.g., to minimize total off-target activity across the genome. Off target activity may be other than cleavage. For each possible gRNA choice e.g., using S. pyogenes Cas9, the tool can identify all off-target sequences (e.g., preceding either NAG or NGG PAMs) across the genome that contain up to certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA is then ranked according to its total predicted off-target cleavage; the top-ranked gRNAs represent those that are likely to have the greatest on-target and the least off-target cleavage. Other functions, e.g., automated reagent design for CRISPR construction, primer design for the on-target Surveyor assay, and primer design for high-throughput detection and quantification of off-target cleavage via next-gen sequencing, can also be included in the tool. Candidate gRNA molecules can be evaluated by art-known methods or as described herein.

Although software algorithms may be used to generate an initial list of potential gRNA molecules, cutting efficiency and specificity will not necessarily reflect the predicted values, and gRNA molecules typically require screening in specific cell lines, e.g., primary human cell lines, e.g., human HSPCs, e.g., human CD34+ cells, to determine, for example, cutting efficiency, indel formation, cutting specificity and change in desired phenotype. These properties may be assayed by the methods described herein.

### IV. Cas Molecules

### Cas9 Molecules

In preferred embodiments, the Cas molecule is a Cas9 molecule. Cas9 molecules of a variety of species can be used in the methods and compositions described herein. While the S. pyogenes Cas9 molecule are the subject of much of the disclosure herein, Cas9 molecules of, derived from, or based on the Cas9 proteins of other species listed herein can be used as well. In other words, other Cas9 molecules, e.g., S. thermophilus, Staphylococcus aureus and/or Neisseria meningitidis Cas9 molecules, may be used in the systems, methods and compositions described herein. Additional Cas9 species include: Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhiz′ obium sp., Brevibacillus latemsporus, Campylobacter coli, Campylobacter jejuni, Campylobacter lad, Candidatus puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, Gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

A Cas9 molecule, as that term is used herein, refers to a molecule that can interact with a gRNA molecule (e.g., sequence of a domain of a tracr) and, in concert with the gRNA molecule, localize (e.g., target or home) to a site which comprises a target sequence and PAM sequence.

In an embodiment, the Cas9 molecule is capable of cleaving a target nucleic acid molecule, which may be referred to herein as an active Cas9 molecule. In an embodiment, an active Cas9 molecule, comprises one or more of the following activities: a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which in an embodiment is the presence of two nickase activities; an endonuclease activity; an exonuclease activity; and a helicase activity, i.e., the ability to unwind the helical structure of a double stranded nucleic acid.

In an embodiment, an enzymatically active Cas9 molecule cleaves both DNA strands and results in a double stranded break. In an embodiment, a Cas9 molecule cleaves only one strand, e.g., the strand to which the gRNA hybridizes to, or the strand complementary to the strand the gRNA hybridizes with. In an embodiment, an active Cas9 molecule comprises cleavage activity associated with an HNH-like domain. In an embodiment, an active Cas9 molecule comprises cleavage activity associated with an N-terminal RuvC-like domain. In an embodiment, an active Cas9 molecule comprises cleavage activity associated with an HNH-like domain and cleavage activity associated with an N-terminal RuvC-like domain. In an embodiment, an active Cas9 molecule comprises an active, or cleavage competent, HNH-like domain and an inactive, or cleavage incompetent, N-terminal RuvC-like domain. In an embodiment, an active Cas9 molecule comprises an inactive, or cleavage incompetent, HNH-like domain and an active, or cleavage competent, N-terminal RuvC-like domain.

In an embodiment, the ability of an active Cas9 molecule to interact with and cleave a target nucleic acid is PAM sequence dependent. A PAM sequence is a sequence in the target nucleic acid. In an embodiment, cleavage of the target nucleic acid occurs upstream from the PAM sequence. Active Cas9 molecules from different bacterial species can recognize different sequence motifs (e.g., PAM sequences). In an embodiment, an active Cas9 molecule of S. pyogenes recognizes the sequence motif NGG and directs cleavage of a target nucleic acid sequence 1 to 10, e.g., 3 to 5, base pairs upstream from that sequence. See, e.g., Mali et al, SCIENCE 2013; 339(6121): 823-826. In an embodiment, an active Cas9 molecule of S. thermophilus recognizes the sequence motif NGGNG and NNAG AAW (W=A or T) and directs cleavage of a core target nucleic acid sequence 1 to 10, e.g., 3 to 5, base pairs upstream from these sequences. See, e.g., Horvath et al., SCIENCE 2010; 327(5962): 167-170, and Deveau et al, J BACTERIOL 2008; 190(4): 1390-1400. In an embodiment, an active Cas9 molecule of S. mulans recognizes the sequence motif NGG or NAAR (R-A or G) and directs cleavage of a core target nucleic acid sequence 1 to 10, e.g., 3 to 5 base pairs, upstream from this sequence. See, e.g., Deveau et al., J BACTERIOL 2008; 190(4): 1390-1400.

In an embodiment, an active Cas9 molecule of S. aureus recognizes the sequence motif NNGRR (R=A or G) and directs cleavage of a target nucleic acid sequence 1 to 10, e.g., 3 to 5, base pairs upstream from that sequence. See, e.g., Ran F. et al., NATURE, vol. 520, 2015, pp. 186-191. In an embodiment, an active Cas9 molecule of N. meningitidis recognizes the sequence motif NNNNGATT and directs cleavage of a target nucleic acid sequence 1 to 10, e.g., 3 to 5, base pairs upstream from that sequence. See, e.g., Hou et al., PNAS EARLY EDITION 2013, 1-6. The ability of a Cas9 molecule to recognize a PAM sequence can be determined, e.g., using a transformation assay described in Jinek et al, SCIENCE 2012, 337:816.

Some Cas9 molecules have the ability to interact with a gRNA molecule, and in conjunction with the gRNA molecule home (e.g., targeted or localized) to a core target domain, but are incapable of cleaving the target nucleic acid, or incapable of cleaving at efficient rates. Cas9 molecules having no, or no substantial, cleavage activity may be referred to herein as an inactive Cas9 (an enzymatically inactive Cas9), a dead Cas9, or a dCas9 molecule. For example, an inactive Cas9 molecule can lack cleavage activity or have substantially less, e.g., less than 20, 10, 5, 1 or 0.1% of the cleavage activity of a reference Cas9 molecule, as measured by an assay described herein.

Exemplary naturally occurring Cas9 molecules are described in Chylinski et al, RNA Biology 2013; 10:5, 727-737. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 1 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. pyogenes (e.g., strain SF370, MGAS 10270, MGAS 10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA 159, NN2025), S. macacae (e.g., strain NCTC1 1558), S. gallolylicus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. cmginosus (e.g.; strain F021 1), S. agalactia (e.g., strain NEM316, A909), Listeria monocytogenes (e.g., strain F6854), Listeria innocua (L. innocua, e.g., strain Clip 11262), Enterococcus italicus (e.g., strain DSM 15952), or Enterococcus faecium (e.g., strain 1,231,408). Additional exemplary Cas9 molecules are a Cas9 molecule of Neisseria meningitidis (Hou et al. PNAS Early Edition 2013, 1-6) and a S. aureus Cas9 molecule.

In an embodiment, a Cas9 molecule, e.g., an active Cas9 molecule or inactive Cas9 molecule, comprises an amino acid sequence: having 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; any Cas9 molecule sequence described herein or a naturally occurring Cas9 molecule sequence, e.g., a Cas9 molecule from a species listed herein or described in Chylinski et al., RNA Biology 2013, 10:5, T2T-1 Hou et al. PNAS Early Edition 2013, 1-6.

In an embodiment, a Cas9 molecule comprises an amino acid sequence having 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; S. pyogenes Cas9 (NCBI Reference Sequence: WP_010922251.1; SEQ ID NO: 3133).

In embodiments, the Cas9 molecule is a S. pyogenes Cas9 variant of SEQ ID NO: 3133 that includes one or more mutations to positively charged amino acids (e.g., lysine, arginine or histidine) that introduce an uncharged or nonpolar amino acid, e.g., alanine, at said position. In embodiments, the mutation is to one or more positively charged amino acids in the nt-groove of Cas9. In embodiments, the Cas9 molecule is a S. pyogenes Cas9 variant of SEQ ID NO: 3133 that includes a mutation at position 855 of SEQ ID NO: 3133, for example a mutation to an uncharged amino acid, e.g., alanine, at position 855 of SEQ ID NO: 3133. In embodiments, the Cas9 molecule has a mutation only at position 855 of SEQ ID NO: 3133, relative to SEQ ID NO: 3133, e.g., to an uncharged amino acid, e.g., alanine. In embodiments, the Cas9 molecule is a S. pyogenes Cas9 variant of SEQ ID NO: 3133 that includes a mutilation at position 810, a mutation at position 1003, and/or a mutation at position 1060 of SEQ ID NO: 3133, for example a mutation to alanine at position 810, position 1003, and/or position 1060 of SEQ ID NO: 3133. In embodiments, the Cas9 molecule has a mutation only at position 810, position 1003, and position 1060 of SEQ ID NO: 3133, relative to SEQ ID NO: 3133, e.g., where each mutation is to an uncharged amino acid, for example, alanine. In embodiments, the Cas9 molecule is a S. pyogenes Cas9 variant of SEQ ID NO: 3133 that includes a mutation at position 848, a mutation at position 1003, and/or a mutation at position 1060 of SEQ ID NO: 3133, for example a mutation to alanine at position 848, position 1003, and/or position 1060 of SEQ ID NO: 3133. In embodiments, the Cas9 molecule has a mutation only at position 848, position 1003, and position 1060 of SEQ ID NO: 3133, relative to SEQ ID NO: 3133, e.g., where each mutation is to an uncharged amino acid, for example, alanine. In embodiments, the Cas9 molecule is a Cas9 molecule as described in Slaymaker et al., Science Express, available online Dec. 1, 2015 at Science DOI: 10.1126/science.aad5227.

In embodiments, the Cas9 molecule is a S. pyogenes Cas9 variant of SEQ ID NO: 3133 that includes one or more mutations. In embodiments, the Cas9 variant comprises a mutation at position 80 of SEQ ID NO: 3133, e.g., includes a leucine at position 80 of SEQ ID NO: 3133 (i.e., comprises, e.g., consists of, SEQ ID NO: 3133 with a C80L mutation). In embodiments, the Cas9 variant comprises a mutation at position 574 of SEQ ID NO: 3133, e.g., includes a glutamic acid at position 574 of SEQ ID NO: 3133 (i.e., comprises, e.g., consists of, SEQ ID NO: 3133 with a C574E mutation). In embodiments, the Cas9 variant comprises a mutation at position 80 and a mutation at position 574 of SEQ ID NO: 3133, e.g., includes a leucine at position 80 of SEQ ID NO: 3133, and a glutamic acid at position 574 of SEQ ID NO: 3133 (i.e., comprises, e.g., consists of, SEQ ID NO: 3133 with a C80L mutation and a C574E mutation). Without being bound by theory, it is believed that such mutations improve the solution properties of the Cas9 molecule.

In embodiments, the Cas9 molecule is a S. pyogenes Cas9 variant of SEQ ID NO: 3133 that includes one or more mutations. In embodiments, the Cas9 variant comprises a mutation at position 147 of SEQ ID NO: 3133, e.g., includes a tyrosine at position 147 of SEQ ID NO: 3133 (i.e., comprises, e.g., consists of, SEQ ID NO: 3133 with a D147Y mutation). In embodiments, the Cas9 variant comprises a mutation at position 411 of SEQ ID NO: 3133, e.g., includes a threonine at position 411 of SEQ ID NO: 3133 (i.e., comprises, e.g., consists of, SEQ ID NO: 3133 with a P411T mutation). In embodiments, the Cas9 variant comprises a mutation at position 147 and a mutation at position 411 of SEQ ID NO: 3133, e.g., includes a tyrosine at position 147 of SEQ ID NO: 3133, and a threonine at position 411 of SEQ ID NO: 3133 (i.e., comprises, e.g., consists of, SEQ ID NO: 3133 with a D147Y mutation and a P411T mutation). Without being bound by theory, it is believed that such mutations improve the targeting efficiency of the Cas9 molecule, e.g., in yeast.

In embodiments, the Cas9 molecule is a S. pyogenes Cas9 variant of SEQ ID NO: 3133 that includes one or more mutations. In embodiments, the Cas9 variant comprises a mutation at position 1135 of SEQ ID NO: 3133, e.g., includes a glutamic acid at position 1135 of SEQ ID NO: 3133 (i.e., comprises, e.g., consists of, SEQ ID NO: 3133 with a D1135E mutation). Without being bound by theory, it is believed that such mutations improve the selectivity of the Cas9 molecule for the NGG PAM sequence versus the NAG PAM sequence.

In embodiments, the Cas9 molecule is a S. pyogenes Cas9 variant of SEQ ID NO: 3133 that includes one or more mutations that introduce an uncharged or nonpolar amino acid, e.g., alanine, at certain positions. In embodiments, the Cas9 molecule is a S. pyogenes Cas9 variant of SEQ ID NO: 3133 that includes a mutation at position 497, a mutation at position 661, a mutation at position 695 and/or a mutation at position 926 of SEQ ID NO: 3133, for example a mutation to alanine at position 497, position 661, position 695 and/or position 926 of SEQ ID NO: 3133. In embodiments, the Cas9 molecule has a mutation only at position 497, position 661, position 695, and position 926 of SEQ ID NO: 3133, relative to SEQ ID NO: 3133, e.g., where each mutation is to an uncharged amino acid, for example, alanine. Without being bound by theory, it is believed that such mutations reduce the cutting by the Cas9 molecule at off-target sites

It will be understood that the mutations described herein to the Cas9 molecule may be combined, and may be combined with any of the fusions or other modifications described herein, and the Cas9 molecule tested in the assays described herein.

Various types of Cas molecules can be used to practice the inventions disclosed herein. In some embodiments, Cas molecules of Type II Cas systems are used. In other embodiments, Cas molecules of other Cas systems are used. For example, Type I or Type III Cas molecules may be used. Exemplary Cas molecules (and Cas systems) are described, e.g., in Haft et ai, PLoS COMPUTATIONAL BIOLOGY 2005, 1(6): e60 and Makarova et al, NATURE REVIEW MICROBIOLOGY 2011, 9:467-477, the contents of both references are incorporated herein by reference in their entirety.

In an embodiment, the Cas9 molecule comprises one or more of the following activities: a nickase activity; a double stranded cleavage activity (e.g., an endonuclease and/or exonuclease activity); a helicase activity; or the ability, together with a gRNA molecule, to localize to a target nucleic acid.

### Altered Cas9 Molecules

Naturally occurring Cas9 molecules possess a number of properties, including: nickase activity, nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In an embodiment, a Cas9 molecules can include all or a subset of these properties. In typical embodiments, Cas9 molecules have the ability to interact with a gRNA molecule and, in concert with the gRNA molecule, localize to a site in a nucleic acid. Other activities, e.g., PAM specificity, cleavage activity, or helicase activity can vary more widely in Cas9 molecules.

Cas9 molecules with desired properties can be made in a number of ways, e.g., by alteration of a parental, e.g., naturally occurring Cas9 molecules to provide an altered Cas9 molecule having a desired property. For example, one or more mutations or differences relative to a parental Cas9 molecule can be introduced. Such mutations and differences comprise: substitutions (e.g., conservative substitutions or substitutions of non-essential amino acids); insertions; or deletions. In an embodiment, a Cas9 molecule can comprises one or more mutations or differences, e.g., at least 1, 2, 3, 4, 5, 10, 15, 20, 30, 40 or 50 mutations but less than 200, 100, or 80 mutations relative to a reference Cas9 molecule. In an embodiment, a mutation or mutations do not have a substantial effect on a Cas9 activity, e.g. a Cas9 activity described herein. In an embodiment, a mutation or mutations have a substantial effect on a Cas9 activity, e.g. a Cas9 activity described herein. In an embodiment, exemplary activities comprise one or more of PAM specificity, cleavage activity, and helicase activity. A mutation(s) can be present, e.g., in: one or more RuvC-like domain, e.g., an N-terminal RuvC-like domain; an HNH-like domain; a region outside the RuvC-like domains and the HNH-like domain. In some embodiments, a mutation(s) is present in an N-terminal RuvC-like domain. In some embodiments, a mutation(s) is present in an HNH-like domain. In some embodiments, mutations are present in both an N-terminal RuvC-like domain and an HNH-like domain.

Whether or not a particular sequence, e.g., a substitution, may affect one or more activity, such as targeting activity, cleavage activity, etc., can be evaluated or predicted, e.g., by evaluating whether the mutation is conservative or by the method described in Section III. In an embodiment, a “non-essential” amino acid residue, as used in the context of a Cas9 molecule, is a residue that can be altered from the wild-type sequence of a Cas9 molecule, e.g., a naturally occurring Cas9 molecule, e.g., an active Cas9 molecule, without abolishing or more preferably, without substantially altering a Cas9 activity (e.g., cleavage activity), whereas changing an “essential” amino acid residue results in a substantial loss of activity (e.g., cleavage activity).

Cas9 Molecules with Altered PAM Recognition or No PAM Recognition

Naturally occurring Cas9 molecules can recognize specific PAM sequences, for example the PAM recognition sequences described above for S. pyogenes, S. thermophilus, S. mutans, S. aureus and N. meningitidis.

In an embodiment, a Cas9 molecule has the same PAM specificities as a naturally occurring Cas9 molecule. In other embodiments, a Cas9 molecule has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule recognizes to decrease off target sites and/or improve specificity; or eliminate a PAM recognition requirement. In an embodiment, a Cas9 molecule can be altered, e.g., to increase length of PAM recognition sequence and/or improve Cas9 specificity to high level of identity to decrease off target sites and increase specificity. In an embodiment, the length of the PAM recognition sequence is at least 4, 5, 6, 7, 8, 9, 10 or 15 amino acids in length. Cas9 molecules that recognize different PAM sequences and/or have reduced off-target activity canbe generated using directed evolution. Exemplary methods and systems that can be used for directed evolution of Cas9 molecules are described, e.g., in Esvelt et al., Nature 2011, 472(7344): 499-503. Candidate Cas9 molecules can be evaluated, e.g., by methods described herein.

### Non-Cleaving and Modified-Cleavage Cas9 Molecules

In an embodiment, a Cas9 molecule comprises a cleavage property that differs from naturally occurring Cas9 molecules, e.g., that differs from the naturally occurring Cas9 molecule having the closest homology. For example, a Cas9 molecule can differ from naturally occurring Cas9 molecules, e.g., a Cas9 molecule of S. pyogenes, as follows: its ability to modulate, e.g., decreased or increased, cleavage of a double stranded break (endonuclease and/or exonuclease activity), e.g., as compared to a naturally occurring Cas9 molecule (e.g., a Cas9 molecule of S. pyogenes); its ability to modulate, e.g., decreased or increased, cleavage of a single strand of a nucleic acid, e.g., a non-complimentary strand of a nucleic acid molecule or a complementary strand of a nucleic acid molecule (nickase activity), e.g., as compared to a naturally occurring Cas9 molecule (e.g., a Cas9 molecule of S. pyogenes); or the ability to cleave a nucleic acid molecule, e.g., a double stranded or single stranded nucleic acid molecule, can be eliminated.

### Modified Cleavage Active Cas9 Molecules

In an embodiment, an active Cas9 molecule comprises one or more of the following activities: cleavage activity associated with an N-terminal RuvC-like domain; cleavage activity associated with an HNH-like domain; cleavage activity associated with an HNH domain and cleavage activity associated with an N-terminal RuvC-like domain.

In an embodiment, the Cas9 molecule is a Cas9 nickase, e.g., cleaves only a single strand of DNA. In an embodiment, the Cas9 nickase includes a mutation at position 10 and/or a mutation at position 840 of SEQ ID NO: 3133, e.g., comprises a D10A and/or H840A mutation to SEQ ID NO: 3133.

### Non-Cleaving Inactive Cas9 Molecules

In an embodiment, the altered Cas9 molecule is an inactive Cas9 molecule which does not cleave a nucleic acid molecule (either double stranded or single stranded nucleic acid molecules) or cleaves a nucleic acid molecule with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, S. thermophilus, S. aureus or N. meningitidis. In an embodiment, the reference Cas9 molecule is the naturally occurring Cas9 molecule having the closest sequence identity or homology. In an embodiment, the inactive Cas9 molecule lacks substantial cleavage activity associated with an N-terminal RuvC-like domain and cleavage activity associated with an HNH-like domain.

In an embodiment, the Cas9 molecule is dCas9 (Tsai et al. (2014), Nat. Biotech. 32:569-577).

A catalytically inactive Cas9 molecule may be fused with a transcription repressor. An inactive Cas9 fusion protein complexes with a gRNA and localizes to a DNA sequence specified by gRNA's targeting domain, but, unlike an active Cas9, it will not cleave the target DNA. Fusion of an effector domain, such as a transcriptional repression domain, to an inactive Cas9 enables recruitment of the effector to any DNA site specified by the gRNA. Site specific targeting of a Cas9 fusion protein to a promoter region of a gene can block or affect polymerase binding to the promoter region, for example, a Cas9 fusion with a transcription factor (e.g., a transcription activator) and/or a transcriptional enhancer binding to the nucleic acid to increase or inhibit transcription activation. Alternatively, site specific targeting of a Cas9-fusion to a transcription repressor to a promoter region of a gene can be used to decrease transcription activation.

Transcription repressors or transcription repressor domains that may be fused to an inactive Cas9 molecule can include ruppel associated box (KRAB or SKD), the Mad mSIN3 interaction domain (SID) or the ERF repressor domain (ERD).

In another embodiment, an inactive Cas9 molecule may be fused with a protein that modifies chromatin. For example, an inactive Cas9 molecule may be fused to heterochromatin protein 1 (HP1), a histone lysine methyltransferase (e.g., SUV39H1, SUV39H2, G9A, ESET/SETDB 1, Pr-SET7/8, SUV4-20H 1,RIZ1), a histone lysine demethylates (e.g., LSD1/BHC1 10, SpLsdl/Sw, 1/Safi 10, Su(var)3-3, JMJD2A/JHDM3A, JMJD2B, JMJD2C/GASCl, JMJD2D, Rph 1, JARID 1 A/RBP2, JARI DIB/PLU-I, JAR1D 1C/SMCX, JARID1 D/SMCY, Lid, Jhn2, Jmj2), a histone lysine deacetylases (e.g., HDAC1, HDAC2, HDAC3, HDAC8, Rpd3, Hos 1, Cir6, HDAC4, HDAC5, HDAC7, HDAC9, Hdal, Cir3, SIRT1, SIRT2, Sir2, Hst1, Hst2, Hst3, Hst4, HDAC 11) and a DNA methylases (DNMT1, DNMT2a/DMNT3b, METI). An inactive Cas9-chomatin modifying molecule fusion protein can be used to alter chromatin status to reduce expression a target gene.

The heterologous sequence (e.g., the transcription repressor domain) may be fused to the N- or C-terminus of the inactive Cas9 protein. In an alternative embodiment, the heterologous sequence (e.g., the transcription repressor domain) may be fused to an internal portion (i.e., a portion other than the N-terminus or C-terminus) of the inactive Cas9 protein.

The ability of a Cas9 molecule/gRNA molecule complex to bind to and cleave a target nucleic acid can be evaluated, e.g., by the methods described herein in Section III. The activity of a Cas9 molecule, e.g., either an active Cas9 or an inactive Cas9, alone or in a complex with a gRNA molecule may also be evaluated by methods well-known in the art, including, gene expression assays and chromatin-based assays, e.g., chromatin immunoprecipitation (ChiP) and chromatin in vivo assay (CiA).

### Other Cas9 Molecule Fusions

In embodiments, the Cas9 molecule, e.g., a Cas9 of S. pyogenes, may additionally comprise one or more amino acid sequences that confer additional activity.

In some aspects, the Cas9 molecule may comprise one or more nuclear localization sequences (NLSs), such as at least 1, 2, 3, 4, 5, 6, 7, 8, 9, 10, or more NLSs. In some embodiments, the Cas9 molecule comprises at least 1, 2, 3, 4, 5, 6, 7, 8, 9, 10, or more NLSs at or near the amino-terminus, at least 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Typically, an NLS consists of one or more short sequences of positively charged lysines or arginines exposed on the protein surface, but other types of NLS are known. Non-limiting examples of NLSs include an NLS sequence comprising or derived from: the NLS of the SV40 virus large T-antigen, having the amino acid sequence PKKKRKV (SEQ ID NO: 3134); the NLS from nucleoplasmin (e.g. the nucleoplasmin bipartite NLS with the sequence KRPAATKKAGQAKKKK (SEQ ID NO: 3135); the c-myc NLS having the amino acid sequence PAAKRVKLD (SEQ ID NO: 3136) or RQRRNELKRSP (SEQ ID NO: 3137); the hRNPA1 M9 NLS having the sequence NQSSNFGPMKGGNFGGRSSGPYGGGGQYFAKPRNQGGY (SEQ ID NO: 3138); the sequence RMRIZFKNKGKDTAELRRRRVEVSVELRKAKKDEQILKRRNV (SEQ ID NO: 3139) of the IBB domain from importin-alpha; the sequences VSRKRPRP (SEQ ID NO: 3140) and PPKKARED (SEQ ID NO: 3141) of the myoma T protein; the sequence PQPKKKPL (SEQ ID NO: 3142) of human p53; the sequence SALIKKKKKMAP (SEQ ID NO: 3143) of mouse c-ab1 IV; the sequences DRLRR (SEQ ID NO: 3144) and PKQKKRK (SEQ ID NO: 3145) of the influenza virus NS1; the sequence RKLKKKIKKL (SEQ ID NO: 3146) of the Hepatitis virus delta antigen; the sequence REKKKFLKRR (SEQ ID NO: 3147) of the mouse Mx1 protein; the sequence KRKGDEVDGVDEVAKKKSKK (SEQ ID NO: 3148) of the human poly(ADP-ribose) polymerase; and the sequence RKCLQAGMNLEARKTKK (SEQ ID NO: 3149) of the steroid hormone receptors (human) glucocorticoid. Other suitable NLS sequences are known in the art (e.g., Sorokin, Biochemistry (Moscow) (2007) 72:13, 1439-1457; Lange J Biol Chem. (2007) 282:8, 5101-5).

In an embodiment, the Cas9 molecule, e.g., S. pyogenes Cas9 molecule, comprises a NLS sequence of SV40, e.g., disposed N terminal to the Cas9 molecule. In an embodiment, the Cas9 molecule, e.g., S. pyogenes Cas9 molecule, comprises a NLS sequence of SV40 disposed N-terminal to the Cas9 molecule and a NLS sequence of SV40 disposed C terminal to the Cas9 molecule. In an embodiment, the Cas9 molecule, e.g., S. pyogenes Cas9 molecule, comprises a NLS sequence of SV40 disposed N-terminal to the Cas9 molecule and a NLS sequence of nucleoplasmin disposed C-terminal to the Cas9 molecule. In any of the aforementioned embodiments, the molecule may additionally comprise a tag, e.g., a His tag, e.g., a His(6) tag (SEQ ID NO: 3175) or His(8) tag (SEQ ID NO: 3176), e.g., at the N terminus or the C terminus.

In some aspects, the Cas9 molecule may comprise one or more amino acid sequences that allow the Cas9 molecule to be specifically recognized, for example a tag. In one embodiment, the tag is a Histidine tag, e.g., a histidine tag comprising at least 3, 4, 5, 6, 7, 8, 9, 10 or more histidine amino acids.

In embodiments, the histidine tag is a His6 tag (six histidines) (SEQ ID NO: 3175). In other embodiments, the histidine tag is a His8 tag (eight histidines) (SEQ ID NO: 3176). In embodiments, the histidine tag may be separated from one or more other portions of the Cas9 molecule by a linker. In embodiments, the linker is GGS. An example of such a fusion is the Cas9 molecule iProt106520.

In some aspects, the Cas9 molecule may comprise one or more amino acid sequences that are recognized by a protease (e.g., comprise a protease cleavage site). In embodiments, the cleavage site is the tobacco etch virus (TEV) cleavage site, e.g., comprises the sequence ENLYFQG (SEQ ID NO: 3158). In some aspects the protease cleavage site, e.g., the TEV cleavage site is disposed between a tag, e.g., a His tag, e.g., a His6 (SEQ ID NO: 3175) or His8 tag (SEQ ID NO: 3176), and the remainder of the Cas9 molecule. Without being bound by theory it is believed that such introduction will allow for the use of the tag for, e.g., purification of the Cas9 molecule, and then subsequent cleavage so the tag does not interfere with the Cas9 molecule function.

In embodiments, the Cas9 molecule (e.g., a Cas9 molecule as described herein) comprises an N-terminal NLS, and a C-terminal NLS (e.g., comprises, from N- to C-terminal NLS-Cas9-NLS), e.g., wherein each NLS is an SV40 NLS (PKKKRKV (SEQ ID NO: 3134)). In embodiments, the Cas9 molecule (e.g., a Cas9 molecule as described herein) comprises an N-terminal NLS, a C-terminal NLS, and a C-terminal His6 tag (SEQ ID NO: 3175) (e.g., comprises, from N- to C-terminal NLS-Cas9-NLS-His tag), e.g., wherein each NLS is an SV40 NLS (PKKKRKV (SEQ ID NO: 3134)). In embodiments, the Cas9 molecule (e.g., a Cas9 molecule as described herein) comprises an N-terminal His tag (e.g., His6 tag (SEQ ID NO: 3175)), an N-terminal NLS, and a C-terminal NLS (e.g., comprises, from N- to C-terminal His tag-NLS-Cas9-NLS), e.g., wherein each NLS is an SV40 NLS (PKKKRKV (SEQ ID NO: 3134)). In embodiments, the Cas9 molecule (e.g., a Cas9 molecule as described herein) comprises an N-terminal NLS and a C-terminal His tag (e.g., His6 tag (SEQ ID NO: 3175)) (e.g., comprises from N- to C-terminal His tag-Cas9-NLS), e.g., wherein the NLS is an SV40 NLS (PKKKRKV (SEQ ID NO: 3134)). In embodiments, the Cas9 molecule (e.g., a Cas9 molecule as described herein) comprises an N-terminal NLS and a C-terminal His tag (e.g., His6 tag (SEQ ID NO: 3175)) (e.g., comprises from N- to C-terminal NLS-Cas9-His tag), e.g., wherein the NLS is an SV40 NLS (PKKKRKV (SEQ ID NO: 3134)). In embodiments, the Cas9 molecule (e.g., a Cas9 molecule as described herein) comprises an N-terminal His tag (e.g., His8 tag (SEQ ID NO: 3176)), an N-terminal cleavage domain (e.g., a tobacco etch virus (TEV) cleavage domain (e.g., comprises the sequence ENLYFQG (SEQ ID NO: 3158))), an N-terminal NLS (e.g., an SV40 NLS; SEQ ID NO: 3134), and a C-terminal NLS (e.g., an SV40 NLS; SEQ ID NO: 3134) (e.g., comprises from N- to C-terminal His tag-TEV-NLS-Cas9-NLS). In any of the aforementioned embodiments the Cas9 has the sequence of SEQ ID NO: 3133.

Alternatively, in any of the aforementioned embodiments, the Cas9 has a sequence of a Cas9 variant of SEQ ID NO: 3133, e.g., as described herein. In any of the aforementioned embodiments, the Cas9 molecule comprises a linker between the His tag and another portion of the molecule, e.g., a GGS linker.

Amino acid sequences of exemplary Cas9 molecules described above are provided below. iProt105026 (also referred to as iProt106154, iProt106331, iProt106545, and PID426303, depending on the preparation of the protein) (SEQ ID NO: 3161):

MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KFRGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTNFDKNL PNEKVLPKHS LLYEYFTVYN ELTKVKYVTE
GMRKPAFLSG EQKKAIVDLL FKTNRKVTVK QLKEDYFKKI ECFDSVEISG VEDRENASLG
TYHDLLKIIK DKDELDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLKDDSI DNKVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDERKDFQF YKVREINNYH HAHDAYLNAV VGTALIKKYP KLESEFVYGD
YKVYDVRKMI AKSEQEIGKA TAKYFFYSNI MNFFKTEITL ANGEIRKRPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH
iProt106518 (SEQ ID NO: 3162):
MAPKKKRKVD KKYSIGLDIG TNSVGWAVIT DEYKVPSKKF KVLGNTDRHS IKKNLIGALL
FDSGETAEAT RLKRTARRRY TRRKNRILYL QEIFSNEMAK VDDSFFHRLE ESFLVEEDKK
HERHPIFGNI VDEVAYHEKY PTIYHLRKKL VDSTDKADLR LIYLALAHMI KERGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTNFDKNL PNEKVLPKHS LLYEYFTVYN ELTKVKYVTE
GMRKPAFLSG EQKKAIVDLL FKTNRKVTVK QLKEDYFKKI EEFDSVEISG VEDRENASLG
TYHDLLKIIK DKDELDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLKDDSI DNKVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDERKDFQF YKVREINNYH HAHDAYLNAV VGTALIKKYP KLESEFVYGD
YKVYDVRKMI AKSEQEIGKA TAKYFFYSNI MNFFKTEITL ANGEIRKRPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH
iProt106519 (SEQ ID NO: 3163):
MGSSHHHHHH HHENLYFQGS MDKKYSIGLD IGTNSVGWAV ITDEYKVPSK KFKVLGNTDR
HSIKKNLIGA LLFDSGETAE ATRLKRTARR RYTRRKNRIC YLQEIFSNEM AKVDDSFFHR
LEESFLVEED KKHERHPIFG NIVDEVAYHE KYPTIYHLRK KLVDSTDKAD LRLIYLALAH
MIKFRGHFLI EGDLNPDNSD VDKLFIQLVQ TYNQLFEENP INASGVDAKA ILSARLSKSR
RLENLIAQLP GEKKNGLFGN LIALSLGLTP NFKSNFDLAE DAKLQLSKDT YDDDLDNLLA
QIGDQYADLF LAAKNLSDAI LLSDILRVNT EITKAPLSAS MIKRYDEHHQ DLTLLKALVR
QQLPEKYKEI FFDQSKNGYA GYIDGGASQE EFYKFIKPIL EKMDGTEELL VKLNREDLLR
KQRTFDNGSI PHQIHLGELH AILRRQEDFY PFLKDNREKI EKILTFRIPY YVGPLARGNS
RFAWMTRKSE ETITPWNFEE VVDKGASAQS FIERMTNEDK NLPNEKVLPK HSLLYEYFTV
YNELTKVKYV TEGMRKPAFL SGEQKKAIVD LLFKTNRKVT VKQLKEDYFK KIECFDSVEI
SGVEDRENAS LGTYHDLLKI IKDKDELDNE ENEDILEDIV LTLTLFEDRE MIEERLKTYA
HLEDDKVMKQ LKRRRYTGWG RLSRKLINGI RDKQSGKTIL DFLKSDGFAN RNFMQLIHDD
SLTFKEDIQK AQVSGQGDSL HEHIANLAGS PAIKKGILQT VKVVDELVKV MGRHKPENIV
IEMARENQTT QKGQKNSRER MKRIEEGIKE LGSQILKEHP VENTQLQNEK LYLYYLQNGR
DMYVDQELDI NRLSDYDVDH IVPQSFLKDD SIDNKVLTRS DKNRGKSDNV PSEEVVKKMK
NYWRQLLNAK LITQRKEDNL TKAERGGLSE LDKAGFIKRQ LVETRQITKH VAQILDSRMN
TKYDENDKLI REVKVITLKS KLVSDFRKDF QFYKVREINN YHHAHDAYLN AVVGTALIKK
YPKLESEFVY GDYKVYDVRK MIAKSEQEIG KATAKYFFYS NIMNFFKTEI TLANGEIRKR
PLIETNGETG EIVWDKGRDF ATVRKVLSMP QVNIVKKTEV QTGGFSKESI LPKRNSDKLI
ARKKDWDPKK YGGFDSPTVA YSVLVVAKVE KGKSKKLKSV KELLGITIME RSSFEKNPID
FLEAKGYKEV KKDLIIKLPK YSLFELENGR KRMLASAGEL QKGNELALPS KYVNFLYLAS
HYEKLKGSPE DNEQKQLFVE QHKHYLDEII EQISEFSKRV ILADANLDKV LSAYNKHRDK
PIREQAENII HLFTLTNLGA PAAFKYEDTT IDRKRYTSTK EVLDATLIHQ SITGLYETRI
DLSQLGGDGG GSPKKKRKV
iProt106520 (SEQ ID NO: 3164):
MAHHHHHHGG SPKKKRKVDK KYSIGLDIGT NSVGWAVITD EYKVPSKKFK VLGNTDRHSI
KKNLIGALLF DSGETAEATR LKRTARRRYT RRKNRICYLQ EIFSNEMAKV DDSFFHRLEE
SFLVEEDKKH ERHPIFGNIV DEVAYHEKYP TIYHLRKKLV DSTDKADLRL IYLALAHMIK
FRGHFLIEGD LNPDNSDVDK LFIQLVQTYN QLFEENPINA SGVDAKAILS ARLSKSRRLE
NLIAQLPGEK KNGLFGNLIA LSLGLTPNFK SNFDLAEDAK LQLSKDTYDD DLDNLLAQIG
DQYADLFLAA KNLSDAILLS DILRVNTEIT KAPLSASMIK RYDEHHQDLT LLKALVRQQL
PEKYKEIFFD QSKNGYAGYI DGGASQEEFY KFIKPILEKM DGTEELLVKL NREDLLRKQR
TFDNGSIPHQ IHLGELHAIL RRQEDFYPFL KDNREKIEKI LTFRIPYYVG PLARGNSRFA
WMTRKSEETI TPWNFEEVVD KGASAQSFIE RMTNFDKNLP NEKVLPKHSL LYEYFTVYNE
LTKVKYVTEG MRKPAFLSGE QKKAIVDLLF KTNRKVTVKQ LKEDYFKKIE CFDSVEISGV
EDRFNASLGT YHDLLKIIKD KDELDNEENE DILEDIVLTL TLFEDREMIE ERLKTYAHLF
DDKVMKQLKR RRYTGWGRLS RKLINGIRDK QSGKTILDFL KSDGFANRNF MQLIHDDSLT
FKEDIQKAQV SGQGDSLHEH IANLAGSPAI KKGILQTVKV VDELVKVMGR HKPENIVIEM
ARENQTTQKG QKNSRERMKR IEEGIKELGS QILKEHPVEN TQLQNEKLYL YYLQNGRDMY
VDQELDINRL SDYDVDHIVP QSFLKDDSID NKVLTRSDKN RGKSDNVPSE EVVKKMKNYW
RQLLNAKLIT QRKFDNLTKA ERGGLSELDK AGFIKRQLVE TRQITKHVAQ ILDSRMNTKY
DENDKLIREV KVITLKSKLV SDFRKDFQFY KVREINNYHH AHDAYLNAVV GTALIKKYPK
LESEFVYGDY KVYDVRKMIA KSEQEIGKAT AKYFFYSNIM NFFKTEITLA NGEIRKRPLI
ETNGETGEIV WDKGRDFATV RKVLSMPQVN IVKKTEVQTG GFSKESILPK RNSDKLIARK
KDWDPKKYGG FDSPTVAYSV LVVAKVEKGK SKKLKSVKEL LGITIMERSS FEKNPIDELE
AKGYKEVKKD LIIKLPKYSL FELENGRKRM LASAGELQKG NELALPSKYV NFLYLASHYE
KLKGSPEDNE QKQLFVEQHK HYLDEIIEQI SEFSKRVILA DANLDKVLSA YNKHRDKPIR
EQAENIIHLF TLTNLGAPAA FKYFDTTIDR KRYTSTKEVL DATLIHQSIT GLYETRIDLS
QLGGDSRADP KKKRKV
iProt106521 (SEQ ID NO: 3165):
MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KERGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTEDNGSIPH
QIHLGELHAI LRRQEDFYPF LKDNREKIEK ILTFRIPYYV GPLARGNSRF AWMTRKSEET
ITPWNFEEVV DKGASAQSFI ERMTNEDKNL PNEKVLPKHS LLYEYFTVYN ELTKVKYVTE
GMRKPAFLSG EQKKAIVDLL FKTNRKVTVK QLKEDYFKKI ECFDSVEISG VEDRENASLG
TYHDLLKIIK DKDFLDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLKDDSI DNKVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDFRKDFQF YKVREINNYH HAHDAYLNAV VGTALIKKYP KLESEFVYGD
YKVYDVRKMI AKSEQEIGKA TAKYFFYSNI MNFFKTEITL ANGEIRKRPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
HHHHHH
iProt106522 (SEQ ID NO: 3166):
MAHHHHHHGG SDKKYSIGLD IGTNSVGWAV ITDEYKVPSK KFKVLGNTDR HSIKKNLIGA
LLFDSGETAE ATRLKRTARR RYTRRKNRIC YLQEIFSNEM AKVDDSFFHR LEESFLVEED
KKHERHPIFG NIVDEVAYHE KYPTIYHLRK KLVDSTDKAD LRLIYLALAH MIKFRGHELI
EGDLNPDNSD VDKLFIQLVQ TYNQLFEENP INASGVDAKA ILSARLSKSR RLENLIAQLP
GEKKNGLFGN LIALSLGLTP NEKSNEDLAE DAKLQLSKDT YDDDLDNLLA QIGDQYADLE
LAAKNLSDAI LLSDILRVNT EITKAPLSAS MIKRYDEHHQ DLTLLKALVR QQLPEKYKEI
FFDQSKNGYA GYIDGGASQE EFYKFIKPIL EKMDGTEELL VKLNREDLLR KQRTEDNGSI
PHQIHLGELH AILRRQEDFY PFLKDNREKI EKILTFRIPY YVGPLARGNS RFAWMTRKSE
ETITPWNFEE VVDKGASAQS FIERMTNEDK NLPNEKVLPK HSLLYEYFTV YNELTKVKYV
TEGMRKPAFL SGEQKKAIVD LLFKTNRKVT VKQLKEDYFK KIECFDSVEI SGVEDRENAS
LGTYHDLLKI IKDKDELDNE ENEDILEDIV LTLTLFEDRE MIEERLKTYA HLEDDKVMKQ
LKRRRYTGWG RLSRKLINGI RDKQSGKTIL DFLKSDGFAN RNFMQLIHDD SLTFKEDIQK
AQVSGQGDSL HEHIANLAGS PAIKKGILQT VKVVDELVKV MGRHKPENIV IEMARENQTT
QKGQKNSRER MKRIEEGIKE LGSQILKEHP VENTQLQNEK LYLYYLQNGR DMYVDQELDI
NRLSDYDVDH IVPQSFLKDD SIDNKVLTRS DKNRGKSDNV PSEEVVKKMK NYWRQLLNAK
LITQRKEDNL TKAERGGLSE LDKAGFIKRQ LVETRQITKH VAQILDSRMN TKYDENDKLI
REVKVITLKS KLVSDFRKDF QFYKVREINN YHHAHDAYLN AVVGTALIKK YPKLESEFVY
GDYKVYDVRK MIAKSEQEIG KATAKYFFYS NIMNFFKTEI TLANGEIRKR PLIETNGETG
EIVWDKGRDF ATVRKVLSMP QVNIVKKTEV QTGGFSKESI LPKRNSDKLI ARKKDWDPKK
YGGFDSPTVA YSVLVVAKVE KGKSKKLKSV KELLGITIME RSSFEKNPID FLEAKGYKEV
KKDLIIKLPK YSLFELENGR KRMLASAGEL QKGNELALPS KYVNFLYLAS HYEKLKGSPE
DNEQKQLFVE QHKHYLDEII EQISEFSKRV ILADANLDKV LSAYNKHRDK PIREQAENII
HLFTLTNLGA PAAFKYFDTT IDRKRYTSTK EVLDATLIHQ SITGLYETRI DLSQLGGDSR
ADPKKKRKV
iProt106658 (SEQ ID NO: 3167):
MGSSHHHHHH HHENLYFQGS MDKKYSIGLD IGTNSVGWAV ITDEYKVPSK KFKVLGNTDR
HSIKKNLIGA LLFDSGETAE ATRLKRTARR RYTRRKNRIC YLQEIFSNEM AKVDDSFFHR
LEESFLVEED KKHERHPIFG NIVDEVAYHE KYPTIYHLRK KLVDSTDKAD LRLIYLALAH
MIKFRGHFLI EGDLNPDNSD VDKLFIQLVQ TYNQLFEENP INASGVDAKA ILSARLSKSR
RLENLIAQLP GEKKNGLFGN LIALSLGLTP NFKSNEDLAE DAKLQLSKDT YDDDLDNLLA
QIGDQYADLF LAAKNLSDAI LLSDILRVNT EITKAPLSAS MIKRYDEHHQ DLTLLKALVR
QQLPEKYKEI FFDQSKNGYA GYIDGGASQE EFYKFIKPIL EKMDGTEELL VKLNREDLLR
KQRTFDNGSI PHQIHLGELH AILRRQEDFY PFLKDNREKI EKILTFRIPY YVGPLARGNS
RFAWMTRKSE ETITPWNFEE VVDKGASAQS FIERMTNEDK NLPNEKVLPK HSLLYEYFTV
YNELTKVKYV TEGMRKPAFL SGEQKKAIVD LLFKTNRKVT VKQLKEDYFK KIECFDSVEI
SGVEDRFNAS LGTYHDLLKI IKDKDELDNE ENEDILEDIV LTLTLFEDRE MIEERLKTYA
HLFDDKVMKQ LKRRRYTGWG RLSRKLINGI RDKQSGKTIL DELKSDGFAN RNFMQLIHDD
SLTFKEDIQK AQVSGQGDSL HEHIANLAGS PAIKKGILQT VKVVDELVKV MGRHKPENIV
IEMARENQTT QKGQKNSRER MKRIEEGIKE LGSQILKEHP VENTQLQNEK LYLYYLQNGR
DMYVDQELDI NRLSDYDVDH IVPQSFLKDD SIDNKVLTRS DKNRGKSDNV PSEEVVKKMK
NYWRQLLNAK LITQRKEDNL TKAERGGLSE LDKAGFIKRQ LVETRQITKH VAQILDSRMN
TKYDENDKLI REVKVITLKS KLVSDFRKDF QFYKVREINN YHHAHDAYLN AVVGTALIKK
YPKLESEFVY GDYKVYDVRK MIAKSEQEIG KATAKYFFYS NIMNFFKTEI TLANGEIRKR
PLIETNGETG EIVWDKGRDF ATVRKVLSMP QVNIVKKTEV QTGGFSKESI LPKRNSDKLI
ARKKDWDPKK YGGFDSPTVA YSVLVVAKVE KGKSKKLKSV KELLGITIME RSSFEKNPID
FLEAKGYKEV KKDLIIKLPK YSLFELENGR KRMLASAGEL QKGNELALPS KYVNFLYLAS
HYEKLKGSPE DNEQKQLFVE QHKHYLDEII EQISEFSKRV ILADANLDKV LSAYNKHRDK
PIREQAENII HLFTLTNLGA PAAFKYFDTT IDRKRYTSTK EVLDATLIHQ SITGLYETRI
DLSQLGGDGG GSPKKKRKV
iProt106745 (SEQ ID NO: 3168):
MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KERGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTNFDKNL PNEKVLPKHS LLYEYFTVYN ELTKVKYVTE
GMRKPAFLSG EQKKAIVDLL FKTNRKVTVK QLKEDYFKKI ECFDSVEISG VEDRENASLG
TYHDLLKIIK DKDELDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLKDDSI DNAVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDERKDFQF YKVREINNYH HAHDAYLNAV VGTALIKKYP KLESEFVYGD
YKVYDVRKMI AKSEQEIGKA TAKYFFYSNI MNFFKTEITL ANGEIRKRPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH
iProt106746 (SEQ ID NO: 3169):
MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KERGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNEDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTNEDKNL PNEKVLPKHS LLYEYFTVYN ELTKVKYVTE
GMRKPAFLSG EQKKAIVDLL FKTNRKVTVK QLKEDYFKKI ECFDSVEISG VEDRENASLG
TYHDLLKIIK DKDELDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEALY LYYLQNGRDM YVDQELDINR
LSDYDVDHIV PQSFLKDDSI DNKVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDFRKDFQF YKVREINNYH HAHDAYLNAV VGTALIKKYP ALESEFVYGD
YKVYDVRKMI AKSEQEIGKA TAKYFFYSNI MNFFKTEITL ANGEIRKAPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDEL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH
iProt106747 (SEQ ID NO: 3170):
MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KERGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTNEDKNL PNEKVLPKHS LLYEYFTVYN ELTKVKYVTE
GMRKPAFLSG EQKKAIVDLL FKTNRKVTVK QLKEDYFKKI ECFDSVEISG VEDRENASLG
TYHDLLKIIK DKDELDNEEN EDILEDIVLT LTLFEDREMI EERLKTYAHL FDDKVMKQLK
RRRYTGWGRL SRKLINGIRD KQSGKTILDF LKSDGFANRN FMQLIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLADDSI DNKVLTRSDK NRGKSDNVPS EEVVKKMKNY WRQLLNAKLI
TQRKFDNLTK AERGGLSELD KAGFIKRQLV ETRQITKHVA QILDSRMNTK YDENDKLIRE
VKVITLKSKL VSDFRKDFQF YKVREINNYH HAHDAYLNAV VGTALIKKYP ALESEFVYGD
YKVYDVRKMI AKSEQEIGKA TAKYFFYSNI MNFFKTEITL ANGEIRKAPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDEL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH
iProt106884 (SEQ ID NO: 3171):
MAPKKKRKVD KKYSIGLDIG TNSVGWAVIT DEYKVPSKKF KVLGNTDRHS IKKNLIGALL
FDSGETAEAT RLKRTARRRY TRRKNRICYL QEIFSNEMAK VDDSFFHRLE ESFLVEEDKK
HERHPIFGNI VDEVAYHEKY PTIYHLRKKL VDSTDKADLR LIYLALAHMI KERGHFLIEG
DLNPDNSDVD KLFIQLVQTY NQLFEENPIN ASGVDAKAIL SARLSKSRRL ENLIAQLPGE
KKNGLFGNLI ALSLGLTPNF KSNFDLAEDA KLQLSKDTYD DDLDNLLAQI GDQYADLFLA
AKNLSDAILL SDILRVNTEI TKAPLSASMI KRYDEHHQDL TLLKALVRQQ LPEKYKEIFF
DQSKNGYAGY IDGGASQEEF YKFIKPILEK MDGTEELLVK LNREDLLRKQ RTFDNGSIPH
QIHLGELHAI LRRQEDFYPF LKDNREKIEK ILTFRIPYYV GPLARGNSRF AWMTRKSEET
ITPWNFEEVV DKGASAQSFI ERMTAFDKNL PNEKVLPKHS LLYEYFTVYN ELTKVKYVTE
GMRKPAFLSG EQKKAIVDLL FKTNRKVTVK QLKEDYFKKI ECFDSVEISG VEDRENASLG
TYHDLLKIIK DKDELDNEEN EDILEDIVLT LTLFEDREMI EERLKTYAHL FDDKVMKQLK
RRRYTGWGAL SRKLINGIRD KQSGKTILDF LKSDGFANRN FMALIHDDSL TFKEDIQKAQ
VSGQGDSLHE HIANLAGSPA IKKGILQTVK VVDELVKVMG RHKPENIVIE MARENQTTQK
GQKNSRERMK RIEEGIKELG SQILKEHPVE NTQLQNEKLY LYYLQNGRDM YVDQELDINR
LSDYDVDHIV PQSFLKDDSI DNKVLTRSDK NRGKSDNVPS EEVVKKMKNY WRQLLNAKLI
TQRKFDNLTK AERGGLSELD KAGFIKRQLV ETRAITKHVA QILDSRMNTK YDENDKLIRE
VKVITLKSKL VSDFRKDFQF YKVREINNYH HAHDAYLNAV VGTALIKKYP KLESEFVYGD
YKVYDVRKMI AKSEQEIGKA TAKYFFYSNI MNFFKTEITL ANGEIRKRPL IETNGETGEI
VWDKGRDFAT VRKVLSMPQV NIVKKTEVQT GGFSKESILP KRNSDKLIAR KKDWDPKKYG
GFDSPTVAYS VLVVAKVEKG KSKKLKSVKE LLGITIMERS SFEKNPIDFL EAKGYKEVKK
DLIIKLPKYS LFELENGRKR MLASAGELQK GNELALPSKY VNFLYLASHY EKLKGSPEDN
EQKQLFVEQH KHYLDEIIEQ ISEFSKRVIL ADANLDKVLS AYNKHRDKPI REQAENIIHL
FTLTNLGAPA AFKYFDTTID RKRYTSTKEV LDATLIHQSI TGLYETRIDL SQLGGDSRAD
PKKKRKVHHH HHH
iProt 20109496 (SEQ ID NO: 3172)
MAPKKKRKVDKKYSIGLDIGTNSVGWAVITDEYKVPSKKFKVLGNTDRHSIKKNLIGALLFDSGETAE
ATRLKRTARRRYTRRKNRILYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTYNQLF
EENPINASGVDAKAILSARLSKSRRLENLIAQLPGEKKNGLFGNLIALSLGLTPNFKSNEDLAEDAKL
QLSKDTYDDDLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPYYVGPLARGNSRFAWMTRK
SEETITPWNFEEVVDKGASAQSFIERMTNEDKNLPNEKVLPKHSLLYEYFTVYNELTKVKYVTEGMRK
PAFLSGEQKKAIVDLLFKTNRKVTVKQLKEDYFKKIEEFDSVEISGVEDRFNASLGTYHDLLKIIKDK
DFLDNEENEDILEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKELGSQILKEHPVENTQLQN
EKLYLYYLQNGRDMYVDQELDINRLSDYDVDHIVPQSFLKDDSIDNKVLTRSDKNRGKSDNVPSEEVV
KKMKNYWRQLLNAKLITQRKFDNLTKAERGGLSELDKAGFIKRQLVETRQITKHVAQILDSRMNTKYD
ENDKLIREVKVITLKSKLVSDFRKDFQFYKVREINNYHHAHDAYLNAVVGTALIKKYPKLESEFVYGD
YKVYDVRKMIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTVAYSVLVVAK
VEKGKSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQSI
TGLYETRIDLSQLGGDSRADHHHHHH

### Nucleic Acids Encoding Cas9 Molecules

Nucleic acids encoding the Cas9 molecules, e.g., an active Cas9 molecule or an inactive Cas9 molecule are provided herein.

Exemplary nucleic acids encoding Cas9 molecules are described in Cong et al, SCIENCE 2013, 399(6121):819-823; Wang et al, CELL 2013, 153(4):910-918; Mali et al., SCIENCE 2013, 399(6121):823-826; Jinek et al, SCIENCE 2012, 337(6096):816-821.

In an embodiment, a nucleic acid encoding a Cas9 molecule can be a synthetic nucleic acid sequence. For example, the synthetic nucleic acid molecule can be chemically modified, e.g., as described in Section XIII. In an embodiment, the Cas9 mRNA has one or more of, e.g., all of the following properties: it is capped, polyadenylated, substituted with 5-methylcytidine and/or pseudouridine.

In addition or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein.

Provided below is an exemplary codon optimized nucleic acid sequence encoding a Cas9 molecule of S. pyogenes.

(SEQ ID NO: 3150)
ATGGATAAAAAGTACAGCATCGGGCTGGACATCGGTACAAACTCAGTGGGGTGGGCCGTGATTACGGA
CGAGTACAAGGTACCCTCCAAAAAATTTAAAGTGCTGGGTAACACGGACAGACACTCTATAAAGAAAA
ATCTTATTGGAGCCTTGCTGTTCGACTCAGGCGAGACAGCCGAAGCCACAAGGTTGAAGCGGACCGCC
AGGAGGCGGTATACCAGGAGAAAGAACCGCATATGCTACCTGCAAGAAATCTTCAGTAACGAGATGGC
AAAGGTTGACGATAGCTTTTTCCATCGCCTGGAAGAATCCTTTCTTGTTGAGGAAGACAAGAAGCACG
AACGGCACCCCATCTTTGGCAATATTGTCGACGAAGTGGCATATCACGAAAAGTACCCGACTATCTAC
CACCTCAGGAAGAAGCTGGTGGACTCTACCGATAAGGCGGACCTCAGACTTATTTATTTGGCACTCGC
CCACATGATTAAATTTAGAGGACATTTCTTGATCGAGGGCGACCTGAACCCGGACAACAGTGACGTCG
ATAAGCTGTTCATCCAACTTGTGCAGACCTACAATCAACTGTTCGAAGAAAACCCTATAAATGCTTCA
GGAGTCGACGCTAAAGCAATCCTGTCCGCGCGCCTCTCAAAATCTAGAAGACTTGAGAATCTGATTGC
TCAGTTGCCCGGGGAAAAGAAAAATGGATTGTTTGGCAACCTGATCGCCCTCAGTCTCGGACTGACCC
CAAATTTCAAAAGTAACTTCGACCTGGCCGAAGACGCTAAGCTCCAGCTGTCCAAGGACACATACGAT
GACGACCTCGACAATCTGCTGGCCCAGATTGGGGATCAGTACGCCGATCTCTTTTTGGCAGCAAAGAA
CCTGTCCGACGCCATCCTGTTGAGCGATATCTTGAGAGTGAACACCGAAATTACTAAAGCACCCCTTA
GCGCATCTATGATCAAGCGGTACGACGAGCATCATCAGGATCTGACCCTGCTGAAGGCTCTTGTGAGG
CAACAGCTCCCCGAAAAATACAAGGAAATCTTCTTTGACCAGAGCAAAAACGGCTACGCTGGCTATAT
AGATGGTGGGGCCAGTCAGGAGGAATTCTATAAATTCATCAAGCCCATTCTCGAGAAAATGGACGGCA
CAGAGGAGTTGCTGGTCAAACTTAACAGGGAGGACCTGCTGCGGAAGCAGCGGACCTTTGACAACGGG
TCTATCCCCCACCAGATTCATCTGGGCGAACTGCACGCAATCCTGAGGAGGCAGGAGGATTTTTATCC
TTTTCTTAAAGATAACCGCGAGAAAATAGAAAAGATTCTTACATTCAGGATCCCGTACTACGTGGGAC
CTCTCGCCCGGGGCAATTCACGGTTTGCCTGGATGACAAGGAAGTCAGAGGAGACTATTACACCTTGG
AACTTCGAAGAAGTGGTGGACAAGGGTGCATCTGCCCAGTCTTTCATCGAGCGGATGACAAATTTTGA
CAAGAACCTCCCTAATGAGAAGGTGCTGCCCAAACATTCTCTGCTCTACGAGTACTTTACCGTCTACA
ATGAACTGACTAAAGTCAAGTACGTCACCGAGGGAATGAGGAAGCCGGCATTCCTTAGTGGAGAACAG
AAGAAGGCGATTGTAGACCTGTTGTTCAAGACCAACAGGAAGGTGACTGTGAAGCAACTTAAAGAAGA
CTACTTTAAGAAGATCGAATGTTTTGACAGTGTGGAAATTTCAGGGGTTGAAGACCGCTTCAATGCGT
CATTGGGGACTTACCATGATCTTCTCAAGATCATAAAGGACAAAGACTTCCTGGACAACGAAGAAAAT
GAGGATATTCTCGAAGACATCGTCCTCACCCTGACCCTGTTCGAAGACAGGGAAATGATAGAAGAGCG
CTTGAAAACCTATGCCCACCTCTTCGACGATAAAGTTATGAAGCAGCTGAAGCGCAGGAGATACACAG
GATGGGGAAGATTGTCAAGGAAGCTGATCAATGGAATTAGGGATAAACAGAGTGGCAAGACCATACTG
GATTTCCTCAAATCTGATGGCTTCGCCAATAGGAACTTCATGCAACTGATTCACGATGACTCTCTTAC
CTTCAAGGAGGACATTCAAAAGGCTCAGGTGAGCGGGCAGGGAGACTCCCTTCATGAACACATCGCGA
ATTTGGCAGGTTCCCCCGCTATTAAAAAGGGCATCCTTCAAACTGTCAAGGTGGTGGATGAATTGGTC
AAGGTAATGGGCAGACATAAGCCAGAAAATATTGTGATCGAGATGGCCCGCGAAAACCAGACCACACA
GAAGGGCCAGAAAAATAGTAGAGAGCGGATGAAGAGGATCGAGGAGGGCATCAAAGAGCTGGGATCTC
AGATTCTCAAAGAACACCCCGTAGAAAACACACAGCTGCAGAACGAAAAATTGTACTTGTACTATCTG
CAGAACGGCAGAGACATGTACGTCGACCAAGAACTTGATATTAATAGACTGTCCGACTATGACGTAGA
CCATATCGTGCCCCAGTCCTTCCTGAAGGACGACTCCATTGATAACAAAGTCTTGACAAGAAGCGACA
AGAACAGGGGTAAAAGTGATAATGTGCCTAGCGAGGAGGTGGTGAAAAAAATGAAGAACTACTGGCGA
CAGCTGCTTAATGCAAAGCTCATTACACAACGGAAGTTCGATAATCTGACGAAAGCAGAGAGAGGTGG
CTTGTCTGAGTTGGACAAGGCAGGGTTTATTAAGCGGCAGCTGGTGGAAACTAGGCAGATCACAAAGC
ACGTGGCGCAGATTTTGGACAGCCGGATGAACACAAAATACGACGAAAATGATAAACTGATACGAGAG
GTCAAAGTTATCACGCTGAAAAGCAAGCTGGTGTCCGATTTTCGGAAAGACTTCCAGTTCTACAAAGT
TCGCGAGATTAATAACTACCATCATGCTCACGATGCGTACCTGAACGCTGTTGTCGGGACCGCCTTGA
TAAAGAAGTACCCAAAGCTGGAATCCGAGTTCGTATACGGGGATTACAAAGTGTACGATGTGAGGAAA
ATGATAGCCAAGTCCGAGCAGGAGATTGGAAAGGCCACAGCTAAGTACTTCTTTTATTCTAACATCAT
GAATTTTTTTAAGACGGAAATTACCCTGGCCAACGGAGAGATCAGAAAGCGGCCCCTTATAGAGACAA
ATGGTGAAACAGGTGAAATCGTCTGGGATAAGGGCAGGGATTTCGCTACTGTGAGGAAGGTGCTGAGT
ATGCCACAGGTAAATATCGTGAAAAAAACCGAAGTACAGACCGGAGGATTTTCCAAGGAAAGCATTTT
GCCTAAAAGAAACTCAGACAAGCTCATCGCCCGCAAGAAAGATTGGGACCCTAAGAAATACGGGGGAT
TTGACTCACCCACCGTAGCCTATTCTGTGCTGGTGGTAGCTAAGGTGGAAAAAGGAAAGTCTAAGAAG
CTGAAGTCCGTGAAGGAACTCTTGGGAATCACTATCATGGAAAGATCATCCTTTGAAAAGAACCCTAT
CGATTTCCTGGAGGCTAAGGGTTACAAGGAGGTCAAGAAAGACCTCATCATTAAACTGCCAAAATACT
CTCTCTTCGAGCTGGAAAATGGCAGGAAGAGAATGTTGGCCAGCGCCGGAGAGCTGCAAAAGGGAAAC
GAGCTTGCTCTGCCCTCCAAATATGTTAATTTTCTCTATCTCGCTTCCCACTATGAAAAGCTGAAAGG
GTCTCCCGAAGATAACGAGCAGAAGCAGCTGTTCGTCGAACAGCACAAGCACTATCTGGATGAAATAA
TCGAACAAATAAGCGAGTTCAGCAAAAGGGTTATCCTGGCGGATGCTAATTTGGACAAAGTACTGTCT
GCTTATAACAAGCACCGGGATAAGCCTATTAGGGAACAAGCCGAGAATATAATTCACCTCTTTACACT
CACGAATCTCGGAGCCCCCGCCGCCTTCAAATACTTTGATACGACTATCGACCGGAAACGGTATACCA
GTACCAAAGAGGTCCTCGATGCCACCCTCATCCACCAGTCAATTACTGGCCTGTACGAAACACGGATC
GACCTCTCTCAACTGGGCGGCGACTAG

Provided below is an exemplary codon optimized nucleic acid sequence encoding a Cas9 molecule including SEQ ID NO: 3172:

(SEQ ID NO: 3151)
ATGGCTCCGAAGAAAAAGCGTAAAGTGGATAAAAAATACAGCATTGGTCTGGACATTGGCACGAACTC
AGTGGGTTGGGCGGTCATCACGGATGAATATAAGGTCCCGTCAAAAAAGTTCAAAGTGCTGGGCAACA
CCGATCGCCATTCGATTAAAAAGAATCTGATCGGCGCGCTGCTGTTTGATAGCGGTGAAACCGCGGAA
GCAACGCGTCTGAAACGTACCGCACGTCGCCGTTACACGCGCCGTAAAAATCGTATTCTGTATCTGCA
GGAAATCTTTAGCAACGAAATGGCGAAAGTTGATGACTCATTTTTCCACCGCCTGGAAGAATCGTTTC
TGGTCGAAGAAGACAAAAAGCATGAACGTCACCCGATTTTCGGTAATATCGTTGATGAAGTCGCGTAC
CATGAAAAATATCCGACGATTTACCATCTGCGTAAAAAACTGGTGGATTCAACCGACAAAGCCGATCT
GCGCCTGATTTACCTGGCACTGGCTCATATGATCAAATTTCGTGGCCACTTCCTGATTGAAGGTGACC
TGAACCCGGATAACTCTGACGTTGATAAGCTGTTCATCCAGCTGGTCCAAACCTATAATCAGCTGTTC
GAAGAAAACCCGATCAATGCAAGTGGCGTTGATGCGAAGGCCATTCTGTCCGCTCGCCTGAGTAAATC
CCGCCGTCTGGAAAACCTGATTGCACAACTGCCGGGCGAAAAGAAAAACGGCCTGTTTGGTAATCTGA
TCGCTCTGTCACTGGGTCTGACGCCGAACTTTAAATCGAATTTCGACCTGGCAGAAGATGCTAAGCTG
CAGCTGAGCAAAGATACCTACGATGACGATCTGGACAACCTGCTGGCGCAAATTGGTGACCAGTATGC
CGACCTGTTTCTGGCGGCCAAAAATCTGTCAGATGCCATTCTGCTGTCGGACATCCTGCGCGTGAACA
CCGAAATCACGAAAGCGCCGCTGTCAGCCTCGATGATTAAACGCTACGATGAACATCACCAGGACCTG
ACCCTGCTGAAAGCACTGGTTCGTCAGCAACTGCCGGAAAAGTACAAGGAAATTTTCTTTGACCAATC
TAAGAACGGCTATGCAGGTTACATCGATGGCGGTGCTAGTCAGGAAGAATTCTACAAGTTCATCAAGC
CGATCCTGGAAAAAATGGATGGCACGGAAGAACTGCTGGTGAAACTGAATCGTGAAGATCTGCTGCGT
AAACAACGCACCTTTGACAACGGCAGCATTCCGCATCAGATCCACCTGGGTGAACTGCATGCGATTCT
GCGCCGTCAGGAAGATTTTTATCCGTTCCTGAAAGACAACCGTGAAAAAATTGAAAAGATCCTGACGT
TTCGCATCCCGTATTACGTTGGCCCGCTGGCGCGTGGTAATAGCCGCTTCGCCTGGATGACCCGCAAA
TCTGAAGAAACCATTACGCCGTGGAACTTTGAAGAAGTGGTTGATAAAGGTGCAAGCGCTCAGTCTTT
TATCGAACGTATGACCAATTTCGATAAAAACCTGCCGAATGAAAAGGTCCTGCCGAAACATAGCCTGC
TGTATGAATACTTTACCGTGTACAACGAACTGACGAAAGTGAAGTATGTTACCGAAGGCATGCGCAAA
CCGGCGTTTCTGTCTGGTGAACAGAAAAAAGCCATTGTGGATCTGCTGTTCAAGACCAATCGTAAAGT
TACGGTCAAACAGCTGAAGGAAGATTACTTCAAAAAGATCGAAGAATTCGACAGCGTGGAAATTTCTG
GCGTTGAAGATCGTTTCAACGCCAGTCTGGGTACCTATCATGACCTGCTGAAGATCATCAAGGACAAG
GATTTTCTGGATAACGAAGAAAATGAAGACATTCTGGAAGATATCGTGCTGACCCTGACGCTGTTCGA
AGATCGTGAAATGATTGAAGAACGCCTGAAAACGTACGCACACCTGTTTGACGATAAAGTTATGAAGC
AGCTGAAACGCCGTCGCTATACCGGCTGGGGTCGTCTGTCTCGCAAACTGATTAATGGCATCCGCGAT
AAGCAAAGTGGTAAAACGATTCTGGATTTCCTGAAATCCGACGGCTTTGCCAACCGTAATTTCATGCA
GCTGATCCATGACGATAGTCTGACCTTTAAGGAAGACATTCAGAAAGCACAAGTGTCAGGCCAGGGTG
ATTCGCTGCATGAACACATTGCGAACCTGGCCGGCTCCCCGGCTATTAAAAAGGGTATCCTGCAGACC
GTCAAAGTCGTGGATGAACTGGTGAAGGTTATGGGCCGTCACAAACCGGAAAACATTGTGATCGAAAT
GGCGCGCGAAAATCAGACCACGCAAAAGGGTCAGAAAAACTCACGTGAACGCATGAAGCGCATTGAAG
AAGGCATCAAAGAACTGGGTTCGCAGATTCTGAAAGAACATCCGGTTGAAAACACCCAGCTGCAAAAT
GAAAAACTGTACCTGTATTACCTGCAAAATGGCCGTGACATGTATGTCGATCAGGAACTGGACATCAA
CCGCCTGAGCGACTATGATGTCGACCACATTGTGCCGCAGAGCTTTCTGAAGGACGATTCTATCGATA
ATAAAGTGCTGACCCGTTCTGATAAGAACCGCGGTAAAAGCGACAATGTTCCGTCTGAAGAAGTTGTC
AAAAAGATGAAGAACTACTGGCGTCAACTGCTGAATGCGAAGCTGATTACGCAGCGTAAATTCGATAA
CCTGACCAAGGCGGAACGCGGCGGTCTGAGTGAACTGGATAAGGCCGGCTTTATCAAACGTCAACTGG
TGGAAACCCGCCAGATTACGAAACATGTTGCCCAGATCCTGGATTCCCGCATGAACACGAAATATGAC
GAAAATGATAAGCTGATTCGTGAAGTCAAAGTGATCACCCTGAAGAGTAAGCTGGTGTCCGATTTCCG
TAAGGACTTTCAGTTCTACAAAGTTCGCGAAATTAACAATTACCATCACGCACACGATGCTTATCTGA
ATGCAGTGGTTGGCACCGCTCTGATCAAAAAGTATCCGAAACTGGAAAGCGAATTTGTGTATGGTGAT
TACAAAGTCTATGACGTGCGCAAGATGATTGCGAAAAGTGAACAGGAAATCGGCAAGGCGACCGCCAA
GTACTTTTTCTATTCCAACATCATGAACTTTTTCAAGACCGAAATCACGCTGGCAAATGGCGAAATTC
GTAAACGCCCGCTGATCGAAACCAACGGCGAAACGGGTGAAATTGTGTGGGATAAAGGTCGTGACTTC
GCGACCGTTCGCAAAGTCCTGTCAATGCCGCAAGTGAATATCGTTAAAAAGACCGAAGTTCAGACGGG
CGGTTTTAGTAAAGAATCCATCCTGCCGAAGCGTAACTCGGATAAACTGATTGCGCGCAAAAAGGATT
GGGACCCGAAAAAGTACGGCGGTTTTGATAGTCCGACCGTTGCATATTCCGTCCTGGTCGTGGCTAAA
GTTGAAAAAGGCAAGAGTAAAAAGCTGAAGTCCGTCAAAGAACTGCTGGGTATTACCATCATGGAACG
TAGCTCTTTTGAAAAGAACCCGATTGACTTCCTGGAAGCCAAGGGCTACAAAGAAGTGAAAAAGGATC
TGATTATCAAGCTGCCGAAATATTCGCTGTTCGAACTGGAAAACGGTCGTAAACGCATGCTGGCAAGC
GCTGGCGAACTGCAGAAGGGTAATGAACTGGCACTGCCGTCTAAATATGTGAACTTTCTGTACCTGGC
TAGCCATTATGAAAAACTGAAGGGTTCTCCGGAAGATAACGAACAGAAGCAACTGTTCGTTGAACAAC
ATAAACACTACCTGGATGAAATCATCGAACAGATCTCAGAATTCTCGAAACGCGTCATTCTGGCGGAT
GCCAATCTGGACAAAGTGCTGAGCGCGTATAACAAGCATCGTGATAAACCGATTCGCGAACAGGCCGA
AAATATTATCCACCTGTTTACCCTGACGAACCTGGGCGCACCGGCAGCTTTTAAATACTTCGATACCA
CGATCGACCGTAAGCGCTATACCAGCACGAAAGAAGTTCTGGATGCTACCCTGATTCATCAGTCAATC
ACCGGTCTGTATGAAACGCGTATTGACCTGAGCCAACTGGGCGGTGATAGCCGTGCCGACCATCACCA
TCACCATCACTAATAG

If the above Cas9 sequences are fused with a peptide or polypeptide at the C-terminus (e.g., an inactive Cas9 fused with a transcription repressor at the C-terminus), it is understood that the stop codon will be removed.

Also provided herein are nucleic acids, vectors and cells for production of a Cas9 molecule, for example a Cas9 molecule described herein. The recombinant production of polypeptide molecules can be accomplished using techniques known to a skilled artisan. Described herein are molecules and methods for the recombinant production of polypeptide molecules, such as Cas9 molecules, e.g., as described herein. As used in connection herewith, “recombinant” molecules and production includes all polypeptides (e.g., Cas9 molecules, for example as described herein) that are prepared, expressed, created or isolated by recombinant means, such as polypeptides isolated from an animal (e.g., a mouse) that is transgenic or transchromosomal for nucleic acid encoding the molecule of interest, a hybridoma prepared therefrom, molecules isolated from a host cell transformed to express the molecule, e.g., from a transfectoma, molecules isolated from a recombinant, combinatorial library, and molecules prepared, expressed, created or isolated by any other means that involve splicing of all or a portion of a gene encoding the molecule (or portion thereof) to other DNA sequences. Recombinant production may be from a host cell, for example, a host cell comprising nucleic acid encoding a molecule described herein, e.g., a Cas9 molecule, e.g., a Cas9 molecule described herein.

Provided herein are nucleic acid molecules encoding a molecule (e.g., Cas9 molecule and/or gRNA molecule), e.g., as described herein. Specifically provided are nucleic acid molecules comprising sequence encoding any one of SEQ ID NO: 3161 to SEQ ID NO: 3172, or encoding a fragment of any of SEQ ID NO: 3161 to SEQ ID NO: 3172, or encoding a polypeptide comprising at least 90%, at least 91%, at least 92%, at least 93%, at least 94%, at least 95%, at least 96%, at least 97%, at least 98%, or at least 99% sequence homology to any of SEQ ID NO: 3161 to SEQ ID NO: 3172.

Provided herein are vectors, e.g., as described herein, comprising any of the above-described nucleic acid molecules. In embodiments, said nucleic acid molecules are operably linked to a promoter, for example a promoter operable in the host cell into which the vector is introduced.

Provided herein are host cells comprising one or more nucleic acid molecules and/or vectors described herein. In embodiments, the host cell is a prokaryotic host cell. In embodiments, the host cell is a eukaryotic host cell. In embodiments, the host cell is a yeast or E. coli cell. In embodiments, the host cell is a mammalian cell, e.g., a human cell. Such host cells may be used for the production of a recombinant molecule described herein, e.g., a Cas9 or gRNA molecule, e.g., as described herein.

### Other Cas Molecules

Any Cas9 variants or Class II CRISPR endonuclease can be used in any compositions and methods described herein.

The term “Cas9 variant” refers to proteins that have at least 80%, 85%, 90%, 95%, 96%, 97%, 98%, 99% or 100% amino acid sequence identity across the whole sequence or a functional portion of the sequence (e.g. a 50, 100, 150 or 200 continuous amino acid portion) compared to wild-type Cas9 protein and have one or more mutations that increase its binding specificity to PAM compared to wild type Cas9 protein. Exemplary Cas9 variants are listed in the Table 6 below.

TABLE 6

| Cas9 Variants | PAM domains | References |
|---|---|---|
| Strep pyogenes (Sp) Cas9 | NGG | Hsu et al. 2014 |
| | | Cell |
| Staph aureus (Sa) Cas9 | NNGRRT or NNGRR | Ran et al. 2015 |
| | NNGGGT, NNGAAT, | Nature |
| | NNGAGT | |
| | (Zetsche) | |
| SpCas9 VQR mutant | NGAG > NGAT = | Kleinstiver et al. |
| (D1135V, R1335Q, T1337R) | NGAA > NGAC | 2015 Nature |
| | NGCG | |
| SpCas9 VRER mutant | NGCG | Kleinstiver et al. |
| (D1135V/G1218R/ | | 2015 Nature |
| R1335E/T1337R) | | |
| SpCas9 D1135E | NGG, greater fidelity, | Kleinstiver et al. |
| | less cutting at | 2015 Nature |
| | NAG and NGA sites | |
| eSpCas9 1.1 mutant | NGG | Slaymaker et al. |
| (K848A/K1003A/R1060A) | | Science 2015 |
| SpCas9 HF1 | NGG | Kleinstiver et al. |
| (Q695A, Q926A, | | 2016 Nature |
| N497A, R661A) | | |
| AsCpf1 | TTTN (5′ of sgRNA) | Zetsche et al. |
| | | 2015 Cell |

A “Cpf1” or “Cpf1 protein” or “Cas12a” as referred to herein includes any of the recombinant or naturally-occurring forms of the Cpf1 (CxxC finger protein 1) endonuclease or variants or homologs thereof that maintain Cpf1 endonuclease enzyme activity (e.g. within at least 50%, 80%, 90%, 95%, 96%, 97%, 98%, 99% or 100% activity compared to Cpf1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cpf1 protein. In embodiments, the Cpf1 protein is substantially identical to the protein identified by the UniProt reference number Q9POU4 or a variant or homolog having substantial identity thereto.

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) may generated in four sequential steps. First, two non-coding RNAs, the pre-crRNA array and tracrRNA, may be transcribed from the CRISPR locus. Second, tracrRNA may hybridize to the direct repeats of pre-crRNA, which is then processed into mature crRNAs containing individual spacer sequences. Third, the mature crRNA:tracrRNA complex may direct Cas9 to the DNA target consisting of the protospacer and the corresponding PAM via heteroduplex formation between the spacer region of the crRNA and the protospacer DNA. Finally, Cas9 may mediate cleavage of target DNA upstream of PAM to create a DSB within the protospacer.

### V. Functional Analysis of Candidate Molecules

Candidate Cas9 molecules, candidate gRNA molecules, candidate Cas9 molecule/gRNA molecule complexes, can be evaluated by art-known methods or as described herein. For example, exemplary methods for evaluating the endonuclease activity of Cas9 molecule are described, e.g., in Jinek et al., SCIENCE 2012; 337(6096):816-821.

### VI. Template Nucleic Acids (for Introduction of Nucleic Acids)

The term “template nucleic acid” or “donor template” as used herein refers to a nucleic acid to be inserted at or near a target sequence that has been modified, e.g., cleaved, by a CRISPR system of the present invention. In an embodiment, nucleic acid sequence at or near the target sequence is modified to have some or all of the sequence of the template nucleic acid, typically at or near cleavage site(s). In an embodiment, the template nucleic acid is single stranded. In an alternate embodiment, the template nucleic acid is double stranded. In an embodiment, the template nucleic acid is DNA, e.g., double stranded DNA. In an alternate embodiment, the template nucleic acid is single stranded DNA.

In embodiments, the template nucleic acid comprises sequence encoding a globin protein, e.g., a beta globin, e.g., comprises a beta globin gene. In an embodiment, the beta globin encoded by the nucleic acid comprises one or more mutations, e.g., anti-sickling mutations. In an embodiment, the beta globin encoded by the nucleic acid comprises the mutation T87Q. In an embodiment, the beta globin encoded by the nucleic acid comprises the mutation G16D. In an embodiment, the beta globin encoded by the nucleic acid comprises the mutation E22A. In an embodiment, the beta globin gene comprises the mutations G16D, E22A and T87Q. In embodiments, the template nucleic acid further comprises one or more regulatory elements, e.g., a promoter (e.g., a human beta globin promoter), a 3′ enhancer, and/or at least a portion of a globin locus control region (e.g., one or more DNAseI hypersensitivity sites (e.g., HS2, HS3 and/or HS4 of the human globin locus)).

In other embodiments, the template nucleic acid comprises sequence encoding a gamma globin, e.g., comprises a gamma globin gene. In embodiments, the template nucleic acid comprises sequence encoding more than one copy of a gamma globin protein, e.g., comprises two or more, e.g., two, gamma globin gene sequences. In embodiments, the template nucleic acid further comprises one or more regulatory elements, e.g., a promotor and/or enhancer.

In an embodiment, the template nucleic acid alters the structure of the target position by participating in a homology directed repair event. In an embodiment, the template nucleic acid alters the sequence of the target position. In an embodiment, the template nucleic acid results in the incorporation of a modified, or non-naturally occurring base into the target nucleic acid.

Mutations in a gene or pathway described herein may be corrected using one of the approaches discussed herein. In an embodiment, a mutation in a gene or pathway described herein is corrected by homology directed repair (HDR) using a template nucleic acid. In an embodiment, a mutation in a gene or pathway described herein is corrected by homologous recombination (HR) using a template nucleic acid. In an embodiment, a mutation in a gene or pathway described herein is corrected by Non-Homologous End Joining (NHEJ) repair using a template nucleic acid. In other embodiments, nucleic acid encoding molecules of interest may be inserted at or near a site modified by a CRISPR system of the present invention. In embodiments, the template nucleic acid comprises regulatory elements, e.g., one or more promotors and/or enhancers, operably linked to the nucleic acid sequence encoding a molecule of interest, e.g., as described herein.

### HDR or HR Repair and Template Nucleic Acids

As described herein, nuclease-induced homology directed repair (HDR) or homologous recombination (HR) can be used to alter a target sequence and correct (e.g., repair or edit) a mutation in the genome. While not wishing to be bound by theory, it is believed that alteration of the target sequence occurs by repair based on a donor template or template nucleic acid. For example, the donor template or the template nucleic acid provides for alteration of the target sequence. It is contemplated that a plasmid donor or linear double stranded template can be used as a template for homologous recombination. It is further contemplated that a single stranded donor template can be used as a template for alteration of the target sequence by alternate methods of homology directed repair (e.g., single strand annealing) between the target sequence and the donor template. Donor template-effected alteration of a target sequence may depend on cleavage by a Cas9 molecule. Cleavage by Cas9 can comprise a double strand break, one single strand break, or two single strand breaks.

In an embodiment, a mutation can be corrected by either a single double-strand break or two single strand breaks. In an embodiment, a mutation can be corrected by providing a template and a CRISPR/Cas9 system that creates (1) one double strand break, (2) two single strand breaks, (3) two double stranded breaks with a break occurring on each side of the target sequence, (4) one double stranded break and two single strand breaks with the double strand break and two single strand breaks occurring on each side of the target sequence, (5) four single stranded breaks with a pair of single stranded breaks occurring on each side of the target sequence, or (6) one single strand break.

### Double Strand Break Mediated Correction

In an embodiment, double strand cleavage is effected by a Cas9 molecule having cleavage activity associated with an HNH-like domain and cleavage activity associated with a RuvC-like domain, e.g., an N-terminal RuvC-like domain, e.g., a wild type Cas9. Such embodiments require only a single gRNA.

### Single Strand Break Mediated Correction

In other embodiments, two single strand breaks, or nicks, are effected by a Cas9 molecule having nickase activity, e.g., cleavage activity associated with an HNH-like domain or cleavage activity associated with an N-terminal RuvC-like domain. Such embodiments require two gRNAs, one for placement of each single strand break. In an embodiment, the Cas9 molecule having nickase activity cleaves the strand to which the gRNA hybridizes, but not the strand that is complementary to the strand to which the gRNA hybridizes. In an embodiment, the Cas9 molecule having nickase activity does not cleave the strand to which the gRNA hybridizes, but rather cleaves the strand that is complementary to the strand to which the gRNA hybridizes.

In an embodiment, the nickase has HNH activity, e.g., a Cas9 molecule having the RuvC activity inactivated, e.g., a Cas9 molecule having a mutation at D10, e.g., the D10A mutation. D10A inactivates RuvC; therefore, the Cas9 nickase has (only) HN H activity and will cut on the strand to which the gRNA hybridizes (e.g., the complementary strand, which does not have the NGG PAM on it). In other embodiments, a Cas9 molecule having an H840, e.g., an H840A, mutation can be used as a nickase. H840A inactivates HNH; therefore, the Cas9 nickase has (only) RuvC activity and cuts on the non-complementary strand (e.g., the strand that has the NGG PAM and whose sequence is identical to the gRNA).

In an embodiment, in which a nickase and two gRNAs are used to position two single strand nicks, one nick is on the + strand and one nick is on the − strand of the target nucleic acid. The PAMs are outwardly facing. The gRNAs can be selected such that the gRNAs are separated by, from about 0-50, 0-100, or 0-200 nucleotides. In an embodiment, there is no overlap between the target sequence that is complementary to the targeting domains of the two gRNAs. In an embodiment, the gRNAs do not overlap and are separated by as much as 50, 100, or 200 nucleotides. In an embodiment, the use of two gRNAs can increase specificity, e.g., by decreasing off-target binding (Ran et al., CELL 2013).

In an embodiment, a single nick can be used to induce HDR. It is contemplated herein that a single nick can be used to increase the ratio of HDR, HR or NHEJ at a given cleavage site.

### Placement of the Double Strand Break or a Single Strand Break Relative to Target Position

The double strand break or single strand break in one of the strands should be sufficiently close to target position such that correction occurs. In an embodiment, the distance is not more than 50, 100, 200, 300, 350 or 400 nucleotides. While not wishing to be bound by theory, it is believed that the break should be sufficiently close to target position such that the break is within the region that is subject to exonuclease-mediated removal during end resection. If the distance between the target position and a break is too great, the mutation may not be included in the end resection and, therefore, may not be corrected, as donor sequence may only be used to correct sequence within the end resection region.

In an embodiment, in which a gRNA (unimolecular (or chimeric) or modular gRNA) and Cas9 nuclease induce a double strand break for the purpose of inducing HDR- or HR-mediated correction, the cleavage site is between 0-200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target position. In an embodiment, the cleavage site is between 0-100 bp (e.g., 0 to 75, 0 to 50, 0 to 25, 25 to 100, 25 to 75, 25 to 50, 50 to 100, 50 to 75 or 75 to 100 bp) away from the target position.

In an embodiment, in which two gRNAs (independently, unimolecular (or chimeric) or modular gRNA) complexing with Cas9 nickases induce two single strand breaks for the purpose of inducing HDR-mediated correction, the closer nick is between 0-200 bp (e.g., 0 to 175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the target position and the two nicks will ideally be within 25-55 bp of each other (e.g., 25 to 50, 25 to 45, 25 to 40, 25 to 35, 25 to 30, 30 to 55, 30 to 50, 30 to 45, 30 to 40, 30 to 35, 35 to 55, 35 to 50, 35 to 45, 35 to 40, 40 to 55, 40 to 50, 40 to 45 bp) and no more than 100 bp away from each other (e.g., no more than 90, 80, 70, 60, 50, 40, 30, 20, 10 or 5 bp away from each other). In an embodiment, the cleavage site is between 0-100 bp (e.g., 0 to 75, 0 to 50, 0 to 25, 25 to 100, 25 to 75, 25 to 50, 50 to 100, 50 to 75 or 75 to 100 bp) away from the target position.

In one embodiment, two gRNAs, e.g., independently, unimolecular (or chimeric) or modular gRNA, are configured to position a double-strand break on both sides of a target position. In an alternate embodiment, three gRNAs, e.g., independently, unimolecular (or chimeric) or modular gRNA, are configured to position a double strand break (i.e., one gRNA complexes with a Cas9 nuclease) and two single strand breaks or paired single stranded breaks (i.e., two gRNAs complex with Cas9 nickases) on either side of the target position (e.g., the first gRNA is used to target upstream (i.e., 5′) of the target position and the second gRNA is used to target downstream (i.e., 3′) of the target position). In another embodiment, four gRNAs, e.g., independently, unimolecular (or chimeric) or modular gRNA, are configured to generate two pairs of single stranded breaks (i.e., two pairs of two gRNAs complex with Cas9 nickases) on either side of the target position (e.g., the first gRNA is used to target upstream (i.e., 5′) of the target position and the second gRNA is used to target downstream (i.e., 3′) of the target position). The double strand break(s) or the closer of the two single strand nicks in a pair will ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp).

In one embodiment, two gRNAs, e.g., independently, unimolecular (or chimeric) or modular gRNA, are configured to position a double-strand break on both sides of a target position. In an alternate embodiment, three gRNAs, e.g., independently, unimolecular (or chimeric) or modular gRNA, are configured to position a double strand break (i.e., one gRNA complexes with a Cas9 nuclease) and two single strand breaks or paired single stranded breaks (i.e., two gRNAs complex with Cas9 nickases) on two target sequences (e.g., the first gRNA is used to target an upstream (i.e., 5′) target sequence and the second gRNA is used to target a downstream (i.e., 3′) target sequence of an insertion site. In another embodiment, four gRNAs, e.g., independently, unimolecular (or chimeric) or modular gRNA, are configured to generate two pairs of single stranded breaks (i.e., two pairs of two gRNAs complex with Cas9 nickases) on either side of an insertion site (e.g., the first gRNA is used to target an upstream (i.e., 5′) target sequence described herein, and the second gRNA is used to target a downstream (i.e., 3′) target sequence described herein). The double strand break(s) or the closer of the two single strand nicks in a pair will ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp).

### Length of the Homology Arms

The homology arm should extend at least as far as the region in which end resection may occur, e.g., in order to allow the resected single stranded overhang to find a complementary region within the donor template. The overall length could be limited by parameters such as plasmid size or viral packaging limits. In an embodiment, a homology arm does not extend into repeated elements, e.g., ALU repeats, LINE repeats. A template may have two homology arms of the same or different lengths.

Exemplary homology arm lengths include at least 25, 50, 100, 250, 500, 750 or 1000 nucleotides.

Target position, as used herein, refers to a site on a target nucleic acid (e.g., the chromosome) that is modified by a Cas9 molecule-dependent process. For example, the target position can be a modified Cas9 molecule cleavage of the target nucleic acid and template nucleic acid directed modification, e.g., correction, of the target position. In an embodiment, a target position can be a site between two nucleotides, e.g., adjacent nucleotides, on the target nucleic acid into which one or more nucleotides is added. The target position may comprise one or more nucleotides that are altered, e.g., corrected, by a template nucleic acid. In an embodiment, the target position is within a target sequence (e.g., the sequence to which the gRNA binds). In an embodiment, a target position is upstream or downstream of a target sequence (e.g., the sequence to which the gRNA binds).

Typically, the template sequence undergoes a breakage mediated or catalyzed recombination with the target sequence. In an embodiment, the template nucleic acid includes sequence that corresponds to a site on the target sequence that is cleaved by a Cas9 mediated cleavage event. In an embodiment, the template nucleic acid includes sequence that corresponds to both, a first site on the target sequence that is cleaved in a first Cas9 mediated event, and a second site on the target sequence that is cleaved in a second Cas9 mediated event.

In an embodiment, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation.

In other embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element. The template nucleic acid can include sequence which, when integrated, results in:
decreasing the activity of a positive control element;
increasing the activity of a positive control element;
decreasing the activity of a negative control element;
increasing the activity of a negative control element;
decreasing the expression of a gene;
increasing the expression of a gene;
increasing resistance to a disorder or disease;
increasing resistance to viral entry;
correcting a mutation or altering an unwanted amino acid residue;
conferring, increasing, abolishing or decreasing a biological property of a gene product, e.g., increasing the enzymatic activity of an enzyme, or increasing the ability of a gene product to interact with another molecule.

The template nucleic acid can include sequence which results in:
a change in sequence of 1, 2, 3, 4, 5, 6, 7, 8, 9, 10, 11, 12 or more nucleotides of the target sequence.

In an embodiment, the template nucleic acid is 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, 220+/−10, 200-300, 300-400, 400-500, 500-600, 600-700, 700-800, 800-900, 900-1000, 1000-2000, 2000-3000 or more than 3000 nucleotides in length.

A template nucleic acid comprises the following components:
[5′ homology arm]-[insertion sequence]-[3′ homology arm].

The homology arms provide for recombination into the chromosome, which can replace the undesired element, e.g., a mutation or signature, with the replacement sequence. In an embodiment, the homology arms flank the most distal cleavage sites.

In an embodiment, the 3′ end of the 5′ homology arm is the position next to the 5′ end of the replacement sequence. In an embodiment, the 5′ homology arm can extend at least 10, 20, 30, 40, 50, 60, 70, 80, 90, 100, 120, 150, 180, 200, 300, 400, 500, 600, 700, 800, 900, 1000, 1500, or 2000 nucleotides 5′ from the 5′ end of the replacement sequence.

In an embodiment, the 5′ end of the 3′ homology arm is the position next to the 3′ end of the replacement sequence. In an embodiment, the 3′ homology arm can extend at least 10, 20, 30, 40, 50, 60, 70, 80, 90, 100, 120, 150, 180, 200, 300, 400, 500, 600, 700, 800, 900, 1000, 1500, or 2000 nucleotides 3′ from the 3′ end of the replacement sequence.

It is contemplated herein that one or both homology arms may be shortened to avoid including certain sequence repeat elements, e.g., Alu repeats, LINE elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

It is contemplated herein that template nucleic acids for correcting a mutation may designed for use as a single-stranded oligonucleotide (ssODN). When using a ssODN, 5′ and 3′ homology arms may range up to about 200 base pairs (bp) in length, e.g., at least 25, 50, 75, 100, 125, 150, 175, or 200 bp in length. Longer homology arms are also contemplated for ssODNs as improvements in oligonucleotide synthesis continue to be made.

### NHEJ Approaches for Gene Targeting

As described herein, nuclease-induced non-homologous end-joining (NHEJ) can be used to target gene-specific knockouts. Nuclease-induced NHEJ can also be used to remove (e.g., delete) sequence in a gene of interest.

While not wishing to be bound by theory, it is believed that, in an embodiment, the genomic alterations associated with the methods described herein rely on nuclease-induced NHEJ and the error-prone nature of the NHEJ repair pathway. NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations may alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein.

The indel mutations generated by NHEJ are unpredictable in nature; however, at a given break site certain indel sequences are favored and are overrepresented in the population. The lengths of deletions can vary widely; most commonly in the 1-50 bp range, but they can easily reach greater than 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it can also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of repair.

Both double strand cleaving Cas9 molecules and single strand, or nickase, Cas9 molecules can be used in the methods and compositions described herein to generate NHEJ-mediated indels. NHEJ-mediated indels targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

### Placement of Double Strand or Single Strand Breaks Relative to the Target Position

In an embodiment, in which a gRNA and Cas9 nuclease generate a double strand break for the purpose of inducing NHEJ-mediated indels, a gRNA, e.g., a unimolecular (or chimeric) or modular gRNA molecule, is configured to position one double-strand break in close proximity to a nucleotide of the target position. In an embodiment, the cleavage site is between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position).

In an embodiment, in which two gRNAs complexing with Cas9 nickases induce two single strand breaks for the purpose of inducing NHEJ-mediated indels, two gRNAs, e.g., independently, unimolecular (or chimeric) or modular gRNA, are configured to position two single-strand breaks to provide for NHEJ repair a nucleotide of the target position. In an embodiment, the gRNAs are configured to position cuts at the same position, or within a few nucleotides of one another, on different strands, essentially mimicking a double strand break. In an embodiment, the closer nick is between 0-30 bp away from the target position (e.g., less than 30, 25, 20, 1, 10, 9, 8, 7, 6, 5, 4, 3, 2 or 1 bp from the target position), and the two nicks are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp). In an embodiment, the gRNAs are configured to place a single strand break on either side of a nucleotide of the target position.

Both double strand cleaving Cas9 molecules and single strand, or nickase, Cas9 molecules can be used in the methods and compositions described herein to generate breaks both sides of a target position. Double strand or paired single strand breaks may be generated on both sides of a target position to remove the nucleic acid sequence between the two cuts (e.g., the region between the two breaks is deleted). In one embodiment, two gRNAs, e.g., independently, unimolecular (or chimeric) or modular gRNA, are configured to position a double-strand break on both sides of a target position (e.g., the first gRNA is used to target upstream (i.e., 5′) of the mutation in a gene or pathway described herein, and the second gRNA is used to target downstream (i.e., 3′) of the mutation in a gene or pathway described herein). In an alternate embodiment, three gRNAs, e.g., independently, unimolecular (or chimeric) or modular gRNA, are configured to position a double strand break (i.e., one gRNA complexes with a Cas9 nuclease) and two single strand breaks or paired single stranded breaks (i.e., two gRNAs complex with Cas9 nickases) on either side of a target position (e.g., the fu st gRNA is used to target upstream (i.e., 5′) of the mutation in a gene or pathway described herein, and the second gRNA is used to target downstream (i.e., 3′) of the mutation in a gene or pathway described herein). In another embodiment, four gRNAs, e.g., independently, unimolecular (or chimeric) or modular gRNA, are configured to generate two pairs of single stranded breaks (i.e., two pairs of two gRNAs complex with Cas9 nickases) on either side of the target position (e.g., the first gRNA is used to target upstream (i.e., 5′) of the mutation in a gene or pathway described herein, and the second gRNA is used to target downstream (i.e., 3′) of the mutation in a gene or pathway described herein). The double strand break(s) or the closer of the two single strand nicks in a pair will ideally be within 0-500 bp of the target position (e.g., no more than 450, 400, 350, 300, 250, 200, 150, 100, 50 or 25 bp from the target position). When nickases are used, the two nicks in a pair are within 25-55 bp of each other (e.g., between 25 to 50, 25 to 45, 25 to 40, 25 to 35, 25 to 30, 50 to 55, 45 to 55, 40 to 55, 35 to 55, 30 to 55, 30 to 50, 35 to 50, 40 to 50, 45 to 50, 35 to 45, or 40 to 45 bp) and no more than 100 bp away from each other (e.g., no more than 90, 80, 70, 60, 50, 40, 30, 20 or 10 bp).

In other embodiments, the insertion of template nucleic acid may be mediated by microhomology end joining (MMEJ). See, e.g., Saksuma et al., “MMEJ-assisted gene knock-in using TALENs and CRISPR-Cas9 with the PITCh systems.” Nature Protocols 11, 118-133 (2016) doi:10.1038/nprot.2015.140 Published online 17 Dec. 2015, the contents of which are incorporated by reference in their entirety.

### VII. Systems Comprising More than One gRNA Molecule

While not intending to be bound by theory, it has been surprisingly shown herein that the targeting of two target sequences (e.g., by two gRNA molecule/Cas9 molecule complexes which each induce a single- or double-strand break at or near their respective target sequences) located in close proximity on a continuous nucleic acid induces excision (e.g., deletion) of the nucleic acid sequence (or at least 80%, 85%, 90%, 95%, 96%, 97%, 98%, 99% of the nucleic acid sequence) located between the two target sequences. In some aspects, the present disclosure provides for the use of two or more gRNA molecules that comprise targeting domains targeting target sequences in close proximity on a continuous nucleic acid, e.g., a chromosome, e.g., a gene or gene locus, including its introns, exons and regulatory elements. The use may be, for example, by introduction of the two or more gRNA molecules, together with one or more Cas9 molecules (or nucleic acid encoding the two or more gRNA molecules and/or the one or more Cas9 molecules) into a cell.

In some aspects, the target sequences of the two or more gRNA molecules are located at least 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 11,000, 12,000, 13,000, 14,000, or 15,000 nucleotides apart on a continuous nucleic acid, but not more than 25,000 nucleotides apart on a continuous nucleic acid. In embodiments, the target sequences are located between about 4000 and about 6000 nucleotides apart. In an embodiment, the target sequences are located about 4000 nucleotides apart. In an embodiment, the target sequences are located about 5000 nucleotides apart. In an embodiment, the target sequences are located about 6000 nucleotides apart.

In some aspects, the plurality of gRNA molecules each target sequences within the same gene or gene locus. In another aspect, the plurality of gRNA molecules each target sequences within 2 or more different genes or gene loci.

In some aspects, the invention provides compositions and cells comprising a plurality, for example, 2 or more, for example, 2, gRNA molecules of the invention, wherein the plurality of gRNA molecules target sequences less than 15,000, less than 14,000, less than 13,000, less than 12,000, less than 11,000, less than 10,000, less than 9,000, less than 8,000, less than 7,000, less than 6,000, less than 5,000, less than 4,000, less than 3,000, less than 2,000, less than 1,000, less than 900, less than 800, less than 700, less than 600, less than 500, less than 400, less than 300, less than 200, less than 100, less than 90, less than 80, less than 70, less than 60, less than 50, less than 40, or less than 30 nucleotides apart. In an embodiment, the target sequences are on the same strand of duplex nucleic acid. In an embodiment, the target sequences are on different strands of duplex nucleic acid.

In one embodiment, the invention provides a method for excising (e.g., deleting) nucleic acid disposed between two gRNA binding sites disposed less than 25,000, less than 20,000, less than 15,000, less than 14,000, less than 13,000, less than 12,000, less than 11,000, less than 10,000, less than 9,000, less than 8,000, less than 7,000, less than 6,000, less than 5,000, less than 4,000, less than 3,000, less than 2,000, less than 1,000, less than 900, less than 800, less than 700, less than 600, less than 500, less than 400, less than 300, less than 200, less than 100, less than 90, less than 80, less than 70, less than 60, less than 50, less than 40, or less than 30 nucleotides apart on the same or different strands of duplex nucleic acid. In an embodiment, the method provides for deletion of more than 50%, more than 60%, more than 70%, more than 80%, more than 85%, more than 86%, more than 87%, more than 88%, more than 89%, more than 90%, more than 91%, more than 92%, more than 93%, more than 94%, more than 95%, more than 96%, more than 97%, more than 98%, more than 99%, or 100% of the nucleotides disposed between the PAM sites associated with each gRNA binding site. In embodiments, the deletion further comprises of one or more nucleotides within one or more of the PAM sites associated with each gRNA binding site. In embodiments, the deletion also comprises one or more nucleotides outside of the region between the PAM sites associated with each gRNA binding site.

In one aspect, the two or more gRNA molecules comprise targeting domains targeting target sequences flanking a gene regulatory element, e.g., a promotor binding site, an enhancer region, or a repressor region, such that excision of the intervening sequence (or a portion of the intervening sequence) causes up- or down-regulation of a gene of interest. In other embodiments, the two or more gRNA molecules comprise targeting domains that target sequences flanking a gene, such that excision of the intervening sequence (or portion thereof) causes deletion of the gene of interest.

In an embodiment, the two or more gRNA molecules each include a targeting domain comprising, e.g., consisting of, atargeting domain sequence of Table 1, e.g., of Table 2 or, e.g., of Table 3. In embodiments, the two or more gRNA molecules each include a targeting domain comprising, e.g., consisting of, the targeting domain of a gRNA molecule which results in at least 15% upregulation in the number of F cells in a population of red blood cells differentiated (e.g., at day 7 following editing) from HSPCs edited by said gRNA ex vivo by the methods described herein. In aspects, the two or more gRNA molecules comprise targeting domains that are complementary with sequences in the same gene or region, e.g., the ZNF644 gene region. In aspects, the two or more gRNA molecules comprise targeting domains that are complementary with sequences of different genes or regions, for example one in the ZNF644 intron region and one in the ZNF644 exon region.

In one aspect, the two or more gRNA molecules comprise targeting domains targeting target sequences flanking a gene regulatory element, e.g., a promotor binding site, an enhancer region, or a repressor region, such that excision of the intervening sequence (or a portion of the intervening sequence) causes up- or down-regulation of a gene of interest. In another aspect, the two or more gRNA molecules comprise targeting domains targeting target sequences flanking a gene, such that excision of the intervening sequence (or a portion of the intervening sequence) causes deletion of the gene of interest. By way of example, the two or more gRNA molecules comprise targeting domains targeting target sequences flanking the ZNF644 gene, such that the ZNF644 gene is excised.

In an embodiment, the two or more gRNA molecules comprise targeting domains that comprise, e.g., consist of, targeting domains selected from Table 1.

In aspects, the two or more gRNA molecules comprise targeting domains comprising, e.g., consisting of, targeting domain sequences listed in Table 2. In aspects, the two or more gRNA molecules comprise targeting domains comprising, e.g., consisting of, targeting domain sequences of gRNAs listed in Table 3.

### VIII. Properties of the gRNA

It has further been surprisingly shown herein that single gRNA molecules may have target sequences in more than one loci (for example, loci with high sequence homology), and that, when such loci are present on the same chromosome, for example, within less than about 15,000 nucleotides, less than about 14,000 nucleotides, less than about 13,000 nucleotides, less than about 12,000 nucleotides, less than about 11,000 nucleotides, less than about 10,000 nucleotides, less than about 9,000 nucleotides, less than about 8,000 nucleotides, less than about 7,000 nucleotides, less than about 6,000 nucleotides, less than about 5,000 nucleotides, less than about 4,000 nucleotides, or less than about 3,000 nucleotides, (e.g., from about 4,000 to about 6,000 nucleotides apart) such a gRNA molecule may result in excision of the intervening sequence (or portion thereof), thereby resulting in a beneficial effect, for example, upregulation of fetal hemoglobin in erythroid cells differentiated from modified HSPCs (as described herein). Thus, in an aspect, the invention provides gRNA molecules which have target sequences at two loci, for example, to loci on the same chromosome, for example, which have target sequences at a ZNF644 intron region and at ZNF644 exon region (for example as described in Tables 1-3). Without begin bound by theory, it is believed that such gRNAs may result in the cutting of the genome at more than one location (e.g., at the target sequence in each of two regions), and that subsequent repair may result in a deletion of the intervening nucleic acid sequence. Again, without being bound by theory, deletion of said intervening sequence may have a desired effect on the expression or function of one or more proteins.

Without being bound by theory, it is believed that some indel patterns may be more advantageous than others. For example, indels which predominantly include insertions and/or deletions which result in a “frameshift mutation” (e.g., 1- or 2-base pair insertion or deletions, or any insertion or deletion where n/3 is not a whole number (where n=the number of nucleotides in the insertion or deletion)) may be beneficial in reducing or eliminating expression of a functional protein. Likewise, indels which predominantly include “large deletions” (deletions of more than 10, 11, 12, 13, 14, 15, 20, 25, or 30 nucleotides, for example, more than 1 kb, more than 2 kb, more than 3 kb, more than 5 kb or more than 10 kb, for example, comprising sequence disposed between a first and second binding site for a gRNA, e.g., as described herein) may also be beneficial in, for example, removing critical regulatory sequences such as promoter binding sites, or altering the structure or function of a locus, which may similarly have an effect on expression of functional protein. While the indel patterns induced by a given gRNA/CRISPR system have surprisingly been found to be consistently reproduced for a given cell type, gRNA and CRISPR system, as described herein, not any single indel structure will inevitably be produced in a given cell upon introduction of a gRNA/CRISPR system.

The invention thus provides for gRNA molecules which create a beneficial indel pattern or structure, for example, which have indel patterns or structures predominantly composed of large deletions. Such gRNA molecules may be selected by assessing the indel pattern or structure created by a candidate gRNA molecule in a test cell (for example, a HEK293 cell) or in the cell of interest, e.g., a HSPC cell by NGS, as described herein. As shown in the Examples, gRNA molecules have been discovered, which, when introduced into the desired cell population, result in a population of cells comprising a significant fraction of the cells having a large deletion at or near the target sequence of the gRNA. In some cases, the rate of large deletion indel formation is as high as 75%, 80%, 85%, 90% or more. The invention thus provides for populations of cells which comprise at least about 40% of cells (e.g., at least about 45%, at least about 50%, at least about 55%, at least about 60%, at least about 65%, at least about 70%, at least about 75%, at least about 80%, at least about 85%, at least about 90%, at least about 95%, or at least about 99%) having a large deletion, e.g., as described herein, at or near the target site of a gRNA molecule described herein. The invention also provides for populations of cells which comprise at least about 50% of cells (e.g., at least about 55%, at least about 60%, at least about 65%, at least about 70%, at least about 75%, at least about 80%, at least about 85%, at least about 90%, at least about 95%, or at least about 99%) having a large deletion, e.g., as described herein, at or near the target site of a gRNA molecule described herein.

The invention thus provides methods of selecting gRNA molecules for use in the therapeutic methods of the invention comprising: 1) providing a plurality of gRNA molecules to a target of interest, 2) assessing the indel pattern or structure created by use of said gRNA molecules, 3) selecting a gRNA molecule that forms an indel pattern or structure composed predominantly of frameshift mutations, large deletions or a combination thereof, and 4) using said selected gRNA in a methods of the invention.

The invention thus provides methods of selecting gRNA molecules for use in the therapeutic methods of the invention comprising: 1) providing a plurality of gRNA molecules to a target of interest, e.g., which have target sequences at more than one location 2) assessing the indel pattern or structure created by use of said gRNA molecules, 3) selecting a gRNA molecule that forms an excision of sequence comprising nucleic acid sequence located between the two target sequences, e.g., in at least about 25% or more of the cells of a population of cells which are exposed to said gRNA molecules, and 4) using said selected gRNA molecule in a methods of the invention.

The invention further provides methods of altering cells, and altered cells, wherein a particular indel pattern is constantly produced with a given gRNA/CRISPR system in that cell type. The indel patterns, including the top 5 most frequently occurring indels observed with the gRNA/CRISPR systems described herein can be determined using the methods of the examples, and are disclosed, for example, in the Examples. As shown in the Examples, populations of cells are generated, wherein a significant fraction of the cells comprises one of the top 5 indels (for example, populations of cells wherein one of the top 5 indels is present in more than 30%, more than 40%, more than 50%, more than 60% or more of the cells of the population. Thus, the invention provides cells, e.g., HSPCs (as described herein), which comprise an indel of any one of the top 5 indels observed with a given gRNA/CRISPR system. Further, the invention provides populations of cells, e.g., HSPCs (as described herein), which when assessed by, for example, NGS, comprise a high percentage of cells comprising one of the top 5 indels described herein for a given gRNA/CRISPR system. When used in connection with indel pattern analysis, a “high percentage” refers to at least about 50% (e.g., at least about 55%, at least about 60%, at least about 65%, at least about 70%, at least about 75%, at least about 80%, at least about 85%, at least about 90%, at least about 95%, or at least about 99%) of the cells of the population comprising one of the top 5 indels described herein for a given gRNA/CRISPR system. In other embodiments, the population of cells comprises at least about 25% (e.g., from about 25% to about 60%, e.g., from about 25% to about 50%, e.g., from about 25% to about 40%, e.g., from about 25% to about 35%) of cells which have one of the top 5 indels described herein for a given gRNA/CRISPR system.

It has also been discovered that certain gRNA molecules do not create indels at off-target sequences (e.g., off-target sequences outside of the ZNF644 gene region) within the genome of the target cell type, or produce indels at off target sites (e.g., off-target sequences outside of the ZNF644 region) at very low frequencies (e.g., <5% of cells within a population) relative to the frequency of indel creation at the target site. Thus, the invention provides for gRNA molecules and CRISPR systems which do not exhibit off-target indel formation in the target cell type, or which produce a frequency of off-target indel formation of less than 5%, for example, an indel at any off-target site outside of the ZNF644 gene region at a frequency of less than 5%. In embodiments, the invention provides gRNA molecules and CRISPR systems which do not exhibit any off target indel formation in the target cell type. Thus, the invention further provides a cell, e.g., a population of cells, e.g., HSPCs, e.g., as described herein, which comprise an indel at or near a target site of a gRNA molecule described herein (e.g., a frameshift indel, or any one of the top 5 indels produced by a given gRNA/CRISPR system, e.g., as described herein), but does not comprise an indel at any off-target site of the gRNA molecule, for example, an indel at any off-target site outside of the ZNF644 gene region. In other embodiments, the invention further provides a population of cells, e.g., HSPCs, e.g., as described herein, which comprises at least 20%, for example at least 30%, for example at least 40%, for example at least 50%, for example at least 60%, for example at least 70%, for example at least 75% of cells which have an indel at or near a target site of a gRNA molecule described herein (e.g., a frameshift indel, or any one of the top 5 indels produced by a given gRNA/CRISPR system, e.g., as described herein), but which comprises less than 5%, e.g., less than 4%, less than 3%, less than 2% or less than 1%, of cells comprising an indel at any off-target site of the gRNA molecule, for example, an indel at any off-target site outside of the ZNF644 gene region. In other embodiments, the invention further provides a population of cells, e.g., HSPCs, e.g., as described herein, which comprises at least 20%, for example at least 30%, for example at least 40%, for example at least 50%, for example at least 60%, for example at least 70%, for example at least 75%, for example at least 80%, for example at least 90%, for example at least 95%, of cells which have an indel within the ZNF644 gene region (e.g., at or near a sequence which is as least 90% homologous to the target sequence of the gRNA), but which comprises less than 5%, e.g., less than 4%, less than 3%, less than 2% or less than 1%, of cells comprising an indel at or near any off-target site outside of the ZNF644 gene region. In embodiments, the off-target indel is formed within a sequence of a gene, e.g., within a coding sequence of a gene. In embodiments no off-target indel is formed within a sequence of a gene, e.g., within a coding sequence of a gene, in the cell of interest, e.g., as described herein.

### IX. Delivery/Constructs

The components, e.g., a Cas9 molecule or gRNA molecule, or both, can be delivered, formulated, or administered in a variety of forms. As a non-limiting example, the gRNA molecule and Cas9 molecule can be formulated (in one or more compositions), directly delivered or administered to a cell in which a genome editing event is desired. Alternatively, nucleic acid encoding one or more components, e.g., a Cas9 molecule or gRNA molecule, or both, can be formulated (in one or more compositions), delivered or administered. In one aspect, the gRNA molecule is provided as DNA encoding the gRNA molecule and the Cas9 molecule is provided as DNA encoding the Cas9 molecule. In one embodiment, the gRNA molecule and Cas9 molecule are encoded on separate nucleic acid molecules. In one embodiment, the gRNA molecule and Cas9 molecule are encoded on the same nucleic acid molecule. In one aspect, the gRNA molecule is provided as RNA and the Cas9 molecule is provided as DNA encoding the Cas9 molecule. In one embodiment, the gRNA molecule is provided with one or more modifications, e.g., as described herein. In one aspect, the gRNA molecule is provided as RNA and the Cas9 molecule is provided as mRNA encoding the Cas9 molecule. In one aspect, the gRNA molecule is provided as RNA and the Cas9 molecule is provided as a protein. In one embodiment, the gRNA and Cas9 molecule are provided as a ribonuclear protein complex (RNP). In one aspect, the gRNA molecule is provided as DNA encoding the gRNA molecule and the Cas9 molecule is provided as a protein.

Delivery, e.g., delivery of the RNP, (e.g., to HSPC cells as described herein) may be accomplished by, for example, electroporation (e.g., as known in the art) or other method that renders the cell membrane permeable to nucleic acid and/or polypeptide molecules. In embodiments, the CRISPR system, e.g., the RNP as described herein, is delivered by electroporation using a 4D-Nucleofector (Lonza), for example, using program CM-137 on the 4D-Nucleofector (Lonza). In embodiments, the CRISPR system, e.g., the RNP as described herein, is delivered by electroporation using a voltage from about 800 volts to about 2000 volts, e.g., from about 1000 volts to about 1800 volts, e.g., from about 1200 volts to about 1800 volts, e.g., from about 1400 volts to about 1800 volts, e.g., from about 1600 volts to about 1800 volts, e.g., about 1700 volts, e.g., at a voltage of 1700 volts. In embodiments, the pulse width/length is from about 10 ms to about 50 ms, e.g., from about 10 ms to about 40 ms, e.g., from about 10 ms to about 30 ms, e.g., from about 15 ms to about 25 ms, e.g., about 20 ms, e.g., 20 ms. In embodiments, 1, 2, 3, 4, 5, or more, e.g., 2, e.g., 1 pulses are used. In an embodiment, the CRISPR system, e.g., the RNP as described herein, is delivered by electroporation using a voltage of about 1700 volts (e.g., 1700 volts), a pulse width of about 20 ms (e.g., 20 ms), using a single (1) pulse. In embodiments, electroporation is accomplished using a Neon electroporator. Additional techniques for rendering the membrane permeable are known in the art and include, for example, cell squeezing (e.g., as described in WO2015/023982 and WO2013/059343, the contents of which are hereby incorporated by reference in their entirety), nanoneedles (e.g., as described in Chiappini et al., Nat. Mat., 14; 532-39, or US2014/0295558, the contents of which are hereby incorporated by reference in their entirety) and nanostraws (e.g., as described in Xie, ACS Nano, 7(5); 4351-58, the contents of which are hereby incorporated by reference in their entirety).

When a component is delivered encoded in DNA the DNA will typically include a control region, e.g., comprising a promoter, to effect expression. Useful promoters for Cas9 molecule sequences include CMV, EF-1alpha, MSCV, PGK, CAG control promoters. Useful promoters for gRNAs include H1, EF-1a and U6 promoters. Promoters with similar or dissimilar strengths can be selected to tune the expression of components. Sequences encoding a Cas9 molecule can comprise a nuclear localization signal (NLS), e.g., an SV40 NLS. In an embodiment, a promoter for a Cas9 molecule or a gRNA molecule can be, independently, inducible, tissue specific, or cell specific.

DNA-Based Delivery of a Cas9 Molecule and or a gRNA Molecule

DNA encoding Cas9 molecules and/or gRNA molecules, can be administered to subjects or delivered into cells by art-known methods or as described herein. For example, Cas9-encoding and/or gRNA-encoding DNA can be delivered, e.g., by vectors (e.g., viral or non-viral vectors), non-vector based methods (e.g., using naked DNA or DNA complexes), or a combination thereof.

In some embodiments, the Cas9- and/or gRNA-encoding DNA is delivered by a vector (e.g., viral vector/virus, plasmid, minicircle or nanoplasmid).

A vector can comprise a sequence that encodes a Cas9 molecule and/or a gRNA molecule. A vector can also comprise a sequence encoding a signal peptide (e.g., for nuclear localization, nucleolar localization, mitochondrial localization), fused, e.g., to a Cas9 molecule sequence. For example, a vector can comprise one or more nuclear localization sequence (e.g., from SV40) fused to the sequence encoding the Cas9 molecule.

One or more regulatory/control elements, e.g., a promoter, an enhancer, an intron, a polyadenylation signal, a Kozak consensus sequence, internal ribosome entry sites (IRES), a 2A sequence, and a splice acceptor or donor can be included in the vectors. In some embodiments, the promoter is recognized by RNA polymerase II (e.g., a CMV promoter). In other embodiments, the promoter is recognized by RNA polymerase III (e.g., a U6 promoter). In some embodiments, the promoter is a regulated promoter (e.g., inducible promoter). In other embodiments, the promoter is a constitutive promoter. In some embodiments, the promoter is a tissue specific promoter. In some embodiments, the promoter is a viral promoter. In other embodiments, the promoter is a non-viral promoter.

In some embodiments, the vector or delivery vehicle is a minicircle. In some embodiments, the vector or delivery vehicle is a nanoplasmid.

In some embodiments, the vector or delivery vehicle is a viral vector (e.g., for generation of recombinant viruses). In some embodiments, the virus is a DNA virus (e.g., dsDNA or ssDNA virus). In other embodiments, the virus is an RNA virus (e.g., an ssRNA virus).

Exemplary viral vectors/viruses include, e.g., retroviruses, lentiviruses, adenovirus, adeno-associated virus (AAV), vaccinia viruses, poxviruses, and herpes simplex viruses. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals.

In some embodiments, the virus infects dividing cells. In other embodiments, the virus infects non-dividing cells. In some embodiments, the virus infects both dividing and non-dividing cells. In some embodiments, the virus can integrate into the host genome. In some embodiments, the virus is engineered to have reduced immunity, e.g., in human. In some embodiments, the virus is replication-competent. In other embodiments, the virus is replication-defective, e.g., having one or more coding regions for the genes necessary for additional rounds of virion replication and/or packaging replaced with other genes or deleted. In some embodiments, the virus causes transient expression of the Cas9 molecule and/or the gRNA molecule. In other embodiments, the virus causes long-lasting, e.g., at least 1 week, 2 weeks, 1 month, 2 months, 3 months, 6 months, 9 months, 1 year, 2 years, or permanent expression, of the Cas9 molecule and/or the gRNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In some embodiments, the Cas9- and/or gRNA-encoding DNA is delivered by a recombinant retrovirus. In some embodiments, the retrovirus (e.g., Moloney murine leukemia vims) comprises a reverse transcriptase, e.g., that allows integration into the host genome. In some embodiments, the retrovirus is replication-competent. In other embodiments, the retrovirus is replication-defective, e.g., having one of more coding regions for the genes necessary for additional rounds of virion replication and packaging replaced with other genes, or deleted.

In some embodiments, the Cas9- and/or gRNA-encoding DNA is delivered by a recombinant lentivirus. For example, the lentivirus is replication-defective, e.g., does not comprise one or more genes required for viral replication.

In some embodiments, the Cas9- and/or gRNA-encoding DNA is delivered by a recombinant adenovirus. In some embodiments, the adenovirus is engineered to have reduced immunity in human.

In some embodiments, the Cas9- and/or gRNA-encoding DNA is delivered by a recombinant AAV. In some embodiments, the AAV can incorporate its genome into that of a host cell, e.g., a target cell as described herein. In some embodiments, the AAV is a self-complementary adeno-associated virus (scAAV), e.g., a scAAV that packages both strands which anneal together to form double stranded DNA. AAV serotypes that may be used in the disclosed methods include, e.g., AAV1, AAV2, modified AAV2 (e.g., modifications at Y444F, Y500F, Y730F and/or S662V), AAV3, modified AAV3 (e.g., modifications at Y705F, Y73 1 F and/or. T492V), AAV4, AAV5, AAV6, modified AAV6 (e.g., modifications at S663V and/or T492V), AAV8. AAV8.2, AAV9, AAV rh 10, and pseudotyped AAV, such as AAV2/8, AAV2/5 and AAV2/6 can also be used in the disclosed methods.

In some embodiments, the Cas9- and/or gRNA-encoding DNA is delivered by a hybrid virus, e.g., a hybrid of one or more of the viruses described herein.

A Packaging cell is used to form a virus particle that is capable of infecting a host or target cell. Such a cell includes a 293 cell, which can package adenovirus, and a W² cell or a PA317 cell, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed. For example, an AAV vector used in gene therapy typically only possesses inverted terminal repeat (ITR) sequences from the AAV genome which are required for packaging and gene expression in the host or target cell. The missing viral functions are supplied in trans by the packaging cell line. Henceforth, the viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In an embodiment, the viral vector has the ability of cell type and/or tissue type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as a peptide ligand, a single chain antibody, a growth factor); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In an embodiment, the viral vector achieves cell type specific expression. For example, a tissue-specific promoter can be constructed to restrict expression of the transgene (Cas 9 and gRNA) in only the target cell. The specificity of the vector can also be mediated by microRNA-dependent control of transgene expression. In an embodiment, the viral vector has increased efficiency of fusion of the viral vector and a target cell membrane. For example, a fusion protein such as fusion-competent hemagglutinin (HA) can be incorporated to increase viral uptake into cells. In an embodiment, the viral vector has the ability of nuclear localization. For example, a virus that requires the breakdown of the cell wall (during cell division) and therefore will not infect a non-diving cell can be altered to incorporate a nuclear localization peptide in the matrix protein of the virus thereby enabling the transduction of non-proliferating cells.

In some embodiments, the Cas9- and/or gRNA-encoding DNA is delivered by a non-vector based method (e.g., using naked DNA or DNA complexes). For example, the DNA can be delivered, e.g., by organically modified silica or silicate (Ormosil), electroporation, gene gun, sonoporation, magnetofection, lipid-mediated transfection, dendrimers, inorganic nanoparticles, calcium phosphates, or a combination thereof.

In some embodiments, the Cas9- and/or gRNA-encoding DNA is delivered by a combination of a vector and a non-vector based method. For example, a virosome comprises a liposome combined with an inactivated virus (e.g., HIV or influenza virus), which can result in more efficient gene transfer, e.g., in a respiratory epithelial cell than either a viral or a liposomal method alone.

In an embodiment, the delivery vehicle is a non-viral vector. In an embodiment, the non-viral vector is an inorganic nanoparticle (e.g., attached to the payload to the surface of the nanoparticle). Exemplary inorganic nanoparticles include, e.g., magnetic nanoparticles (e.g., Fe lvlnO₂), or silica. The outer surface of the nanoparticle can be conjugated with a positively charged polymer (e.g., polyethylenimine, polylysine, polyserine) which allows for attachment (e.g., conjugation or entrapment) of payload. In an embodiment, the non-viral vector is an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating.

Exemplary lipids and/or polymers for transfer of CRISPR systems or nucleic acid, e.g., vectors, encoding CRISPR systems or components thereof include, for example, those described in WO2011/076807, WO2014/136086, WO2005/060697, WO2014/140211, WO2012/031046, WO2013/103467, WO2013/006825, WO2012/006378, WO2015/095340, and WO2015/095346, the contents of each of the foregoing are hereby incorporated by reference in their entirety. In an embodiment, the vehicle has targeting modifications to increase target cell update of nanoparticles and liposomes, e.g., cell specific antigens, monoclonal antibodies, single chain antibodies, aptamers, polymers, sugars, and cell penetrating peptides. In an embodiment, the vehicle uses fusogenic and endosome-destabilizing peptides/polymers. In an embodiment, the vehicle undergoes acid-triggered conformational changes (e.g., to accelerate endosomal escape of the cargo). In an embodiment, a stimuli-cleavable polymer is used, e.g., for release in a cellular compartment. For example, disulfide-based cationic polymers that are cleaved in the reducing cellular environment can be used.

In an embodiment, the delivery vehicle is a biological non-viral delivery vehicle. In an embodiment, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific tissues, bacteria having modified surface proteins to alter target tissue specificity). In an embodiment, the vehicle is a genetically modified bacteriophage (e.g., engineered phages having large packaging capacity, less immunogenic, containing mammalian plasmid maintenance sequences and having incorporated targeting ligands). In an embodiment, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In an embodiment, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject (i.e., patient) derived membrane-bound nanovesicle (30-100 nm) of endocytic origin (e.g., can be produced from various cell types and can therefore be taken up by cells without the need of for targeting ligands).

In an embodiment, one or more nucleic acid molecules (e.g., DNA molecules) other than the components of a Cas system, e.g., the Cas9 molecule component and/or the gRNA molecule component described herein, are delivered. In an embodiment, the nucleic acid molecule is delivered at the same time as one or more of the components of the Cas system are delivered. In an embodiment, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Cas9 system are delivered. In an embodiment, the nucleic acid molecule is delivered by a different means than one or more of the components of the Cas9 system, e.g., the Cas9 molecule component and/or the gRNA molecule component, are delivered. The nucleic acid molecule can be delivered by any of the delivery methods described herein. For example, the nucleic acid molecule can be delivered by a viral vector, e.g., an integration-deficient lentivirus, and the Cas9 molecule component and/or the gRNA molecule component can be delivered by electroporation, e.g., such that the toxicity caused by nucleic acids (e.g., DNAs) can be reduced. In an embodiment, the nucleic acid molecule encodes a therapeutic protein, e.g., a protein described herein. In an embodiment, the nucleic acid molecule encodes an RNA molecule, e.g., an RNA molecule described herein.

### Delivery of RNA Encoding a Cas9 Molecule

RNA encoding Cas9 molecules (e.g., active Cas9 molecules, inactive Cas9 molecules or inactive Cas9 fusion proteins) and/or gRNA molecules, can be delivered into cells, e.g., target cells described herein, by art-known methods or as described herein. For example, Cas9-encoding and/or gRNA-encoding RNA can be delivered, e.g., by microinjection, electroporation, lipid-mediated transfection, peptide-mediated delivery, or a combination thereof.

### Delivery of Cas9 Molecule as Protein

Cas9 molecules (e.g., active Cas9 molecules, inactive Cas9 molecules or inactive Cas9 fusion proteins) can be delivered into cells by art-known methods or as described herein. For example, Cas9 protein molecules can be delivered, e.g., by microinjection, electroporation, lipid-mediated transfection, peptide-mediated delivery, cell squeezing or abrasion (e.g., by nanoneedles) or a combination thereof. Delivery can be accompanied by DNA encoding a gRNA or by a gRNA, e.g., by precomplexing the gRNA and the Cas9 protein in a ribonuclear protein complex (RNP).

In an aspect the Cas9 molecule, e.g., as described herein, is delivered as a protein and the gRNA molecule is delivered as one or more RNAs (e.g., as a dgRNA or sgRNA, as described herein). In embodiments, the Cas9 protein is complexed with the gRNA molecule prior to delivery to a cell, e.g., as described herein, as a ribonuclear protein complex (“RNP”). In embodiments, the RNP can be delivered into cells, e.g., described herein, by any art-known method, e.g., electroporation. As described herein, and without being bound by theory, it can be preferable to use a gRNA molecule and Cas9 molecule which result in high % editing at the target sequence (e.g., >85%, >90%, >95%, >98%, or >99%) in the target cell, e.g., described herein, even when the concentration of RNP delivered to the cell is reduced. Again, without being bound by theory, delivering a reduced or low concentration of RNP comprising a gRNA molecule that produces a high % editing at the target sequence in the target cell (including at the low RNP concentration), can be beneficial because it may reduce the frequency and number of off-target editing events. In one aspect, where a low or reduced concentration of RNP is to be used, the following exemplary procedure can be used to generate the RNP with a dgRNA molecule:
1. Provide the Cas9 molecule and the tracr in solution at a high concentration (e.g., a concentration higher than the final RNP concentration to be delivered to the cell), and allow the two components to equilibrate;
2. Provide the crRNA molecule, and allow the components to equilibrate (thereby forming a high-concentration solution of the RNP);
3. Dilute the RNP solution to the desired concentration;
4. Deliver said RNP at said desired concentration to the target cells, e.g., by electroporation.

The above procedure may be modified for use with sgRNA molecules by omitting step 2, above, and in step 1, providing the Cas9 molecule and the sgRNA molecule in solution at high concentration, and allowing the components to equilibrate. In embodiments, the Cas9 molecule and each gRNA component are provided in solution at a 1:2 ratio (Cas9:gRNA), e.g., a 1:2 molar ratio of Cas9:gRNA molecule. Where dgRNA molecules are used, the ratio, e.g., molar ratio, is 1:2:2 (Cas9:tracr:crRNA). In embodiments, the RNP is formed at a concentration of 20 uM or higher, e.g., a concentration from about 20 uM to about 50 uM. In embodiments, the RNP is formed at a concentration of 10 uM or higher, e.g., a concentration from about 10 uM to about 30 uM. In embodiments, the RNP is diluted to a final concentration of 10 uM or less (e.g., a concentration from about 0.01 uM to about 10 uM) in a solution comprising the target cell (e.g., described herein) for delivery to said target cell. In embodiments, the RNP is diluted to a final concentration of 3 uM or less (e.g., a concentration from about 0.01 uM to about 3 uM) in a solution comprising the target cell (e.g., described herein) for delivery to said target cell. In embodiments, the RNP is diluted to a final concentration of 1 uM or less (e.g., a concentration from about 0.01 uM to about 1 uM) in a solution comprising the target cell (e.g., described herein) for delivery to said target cell. In embodiments, the RNP is diluted to a final concentration of 0.3 uM or less (e.g., a concentration from about 0.01 uM to about 0.3 uM) in a solution comprising the target cell (e.g., described herein) for delivery to said target cell. In embodiments, the RNP is provided at a final concentration of about 3 uM in a solution comprising the target cell (e.g., described herein) for delivery to said target cell. In embodiments, the RNP is provided at a final concentration of about 2 uM in a solution comprising the target cell (e.g., described herein) for delivery to said target cell. In embodiments, the RNP is provided at a final concentration of about 1 uM in a solution comprising the target cell (e.g., described herein) for delivery to said target cell. In embodiments, the RNP is provided at a final concentration of about 0.3 uM in a solution comprising the target cell (e.g., described herein) for delivery to said target cell. In embodiments, the RNP is provided at a final concentration of about 0.1 uM in a solution comprising the target cell (e.g., described herein) for delivery to said target cell. In embodiments, the RNP is provided at a final concentration of about 0.05 uM in a solution comprising the target cell (e.g., described herein) for delivery to said target cell. In embodiments, the RNP is provided at a final concentration of about 0.03 uM in a solution comprising the target cell (e.g., described herein) for delivery to said target cell. In embodiments, the RNP is provided at a final concentration of about 0.01 uM in a solution comprising the target cell (e.g., described herein) for delivery to said target cell. In embodiments, the RNP is formulated in a medium suitable for electroporation. In embodiments, the RNP is delivered to cells, e.g., HSPC cells, e.g., as described herein, by electroporation, e.g., using electroporation conditions described herein.

In aspects, the components of the gene editing system (e.g., CRISPR system) and/or nucleic acid encoding one or more components of the gene editing system (e.g., CRISPR system) are introduced into the cells by mechanically perturbing the cells, for example, by passing said cells through a pore or channel which constricts the cells. Such perturbation may be accomplished in a solution comprising the components of the gene editing system (e.g., CRISPR system) and/or nucleic acid encoding one or more components of the gene editing system (e.g., CRISPR system), e.g., as described herein. In embodiments, the perturbation is accomplished using a TRIAMF system, e.g., as described herein, for example, in the Examples and in PCT patent application PCT/US17/54110 (incorporated herein by reference in its entirety).

### Bi-Modal or Differential Delivery of Components

Separate delivery of the components of a Cas system, e.g., the Cas9 molecule component and the gRNA molecule component, and more particularly, delivery of the components by differing modes, can enhance performance, e.g., by improving tissue specificity and safety.

In an embodiment, the Cas9 molecule and the gRNA molecule are delivered by different modes, or as sometimes referred to herein as differential modes. Different or differential modes, as used herein, refer modes of delivery that confer different pharmacodynamic or pharmacokinetic properties on the subject component molecule, e.g., a Cas9 molecule, gRNA molecule, or template nucleic acid. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result-in more persistent expression of and presence of a component.

### X. Methods of Treatment

Without being bound by theory, the invention here is based in part on the surprising finding of the linkage between ZNF644 gene expression/protein activity and the hemoglobin F (HbF) production. As demonstrated in the examples and figures, knocking down or knocking out ZNF644 gene or ZNF644 protein in cells (by various modalities/compositions described herein) significantly increased HbF induction in those cells, thereby treating HbF-associated conditions and disorders (e.g., hemoglobinopathies, e.g., sickle cell disease and beta thalassemia).

The Cas9 systems, e.g., one or more gRNA molecules and one or more Cas9 molecules, described herein are useful for the treatment of disease in a mammal, e.g., in a human. The terms “treat,” “treated,” “treating,” and “treatment,” include the administration of cas9 systems, e.g., one or more gRNA molecules and one or more cas9 molecules, to cells to prevent or delay the onset of the symptoms, complications, or biochemical indicia of a disease, alleviating the symptoms or arresting or inhibiting further development of the disease, condition, or disorder. Treatment may also include the administration of one or more (e.g., a population of) cells, e.g., HSPCs, that have been modified by the introduction of a gRNA molecule (or more than one gRNA molecule) of the present invention, or by the introduction of a CRISPR system as described herein, or by any of the methods of preparing said cells described herein, to prevent or delay the onset of the symptoms, complications, or biochemical indicia of a disease, alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease. Treatment can be measured by the therapeutic measures described herein. Thus, the methods of “treatment” of the present invention also include administration of cells altered by the introduction of a cas9 system (e.g., one or more gRNA molecules and one or more Cas9 molecules) into said cells to a subject in order to cure, reduce the severity of, or ameliorate one or more symptoms of a disease or condition, in order to prolong the health or survival of a subject beyond that expected in the absence of such treatment. For example, “treatment” includes the alleviation of a disease symptom in a subject by at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or more.

Cas9 systems comprising gRNA molecules comprising the targeting domains described herein, e.g., in Table 1, and the methods and cells (e.g., as described herein) are useful for the treatment of hemoglobinopathies.

### Delivery Timing

In an embodiment, one or more nucleic acid molecules (e.g., DNA molecules) other than the components of a Cas system, e.g., the Cas9 molecule component and/or the gRNA molecule component described herein, are delivered. In an embodiment, the nucleic acid molecule is delivered at the same time as one or more of the components of the Cas system are delivered. In an embodiment, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In an embodiment, the nucleic acid molecule is delivered by a different means than one or more of the components of the Cas system, e.g., the Cas9 molecule component and/or the gRNA molecule component, are delivered. The nucleic acid molecule can be delivered by any of the delivery methods described herein. For example, the nucleic acid molecule can be delivered by a viral vector, e.g., an integration-deficient lentivirus, and the Cas9 molecule component and/or the gRNA molecule component can be delivered by electroporation, e.g., such that the toxicity caused by nucleic acids (e.g., DNAs) can be reduced. In an embodiment, the nucleic acid molecule encodes a therapeutic protein, e.g., a protein described herein. In an embodiment, the nucleic acid molecule encodes an RNA molecule, e.g., an RNA molecule described herein.

### Bi-Modal or Differential Delivery of Components

Separate delivery of the components of a Cas system, e.g., the Cas9 molecule component and the gRNA molecule component, and more particularly, delivery of the components by differing modes, can enhance performance, e.g., by improving tissue specificity and safety. In an embodiment, the Cas9 molecule and the gRNA molecule are delivered by different modes, or as sometimes referred to herein as differential modes. Different or differential modes, as used herein, refer modes of delivery, that confer different pharmacodynamic or pharmacokinetic properties on the subject component molecule, e.g., a Cas9 molecule, gRNA molecule, template nucleic acid, or payload. E.g., the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., adeno associated virus or lentivirus, delivery.

By way of example, the components, e.g., a Cas9 molecule and a gRNA molecule, can be delivered by modes that differ in terms of resulting half-life or persistent of the delivered component the body, or in a particular compartment, tissue or organ. In an embodiment, a gRNA molecule can be delivered by such modes. The Cas9 molecule component can be delivered by a mode which results in less persistence or less exposure of its to the body or a particular compartment or tissue or organ.

More generally, in an embodiment, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In an embodiment, the first pharmacodynamic or pharmacokinetic property, e.g., distribution, persistence or exposure, is more limited than the second pharmacodynamic or pharmacokinetic property.

In an embodiment, the first mode of delivery is selected to optimize, e.g., minimize, a pharmacodynamic or pharmacokinetic property, e.g., distribution, persistence or exposure.

In an embodiment, the second mode of delivery is selected to optimize, e.g., maximize, a pharmacodynamic or pharmacokinetic property, e.g., distribution, persistence or exposure.

In an embodiment, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In an embodiment, the second mode of delivery comprises a relatively transient element, e.g., an RNA or protein.

In an embodiment, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a Cas9 molecule, is delivered in a transient manner, for example as mRNA or as protein, ensuring that the full Cas9 molecule/gRNA molecule complex is only present and active for a short period of time.

Furthermore, the components can be delivered in different molecular form or with different delivery vectors that complement one another to enhance safety and tissue specificity.

Use of differential delivery modes can enhance performance, safety and efficacy. For example, the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation of active complex is minimized outside the overlap of the target regions. Thus, in an embodiment, a first component, e.g., a gRNA molecule is delivered by a first delivery mode that results in a first spatial, e.g., tissue, distribution. A second component, e.g., a Cas9 molecule is delivered by a second delivery mode that results in a second spatial, e.g., tissue, distribution. In an embodiment, the first mode comprises a first element selected from a liposome, nanoparticle, e.g., polymeric nanoparticle, and a nucleic acid, e.g., viral vector. The second mode comprises a second element selected from the group. In an embodiment, the first mode of delivery comprises a first targeting element, e.g., a cell specific receptor or an antibody, and the second mode of delivery does not include that element. In an embodiment, the second mode of delivery comprises a second targeting element, e.g., a second cell specific receptor or second antibody.

When the Cas9 molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA molecule and the Cas9 molecule are packaged in separated delivery vehicles with distinct but overlapping tissue tropism, the fully functional complex is only be formed in the tissue that is targeted by both vectors.

Candidate Cas molecules, e.g., Cas9 molecules, candidate gRNA molecules, candidate Cas9 molecule/gRNA molecule complexes, and candidate CRISPR systems, can be evaluated by art-known methods or as described herein. For example, exemplary methods for evaluating the endonuclease activity of Cas9 molecule are described, e.g., in Jinek et al., SCIENCE 2012; 337(6096):816-821.

### Hemoglobinopathies

Hemoglobinopathies encompass a number of anemias of genetic origin in which there is a decreased production and/or increased destruction (hemolysis) of red blood cells (RBCs). These also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickle) hemoglobin (HbS). HbS is prone to polymerization, particularly under deoxygenated conditions. HbS RBCs are more fragile than normal RBCs and undergo hemolysis more readily, leading eventually to anemia.

In an embodiment, a hemoglobinopathies-associated gene is targeted, using the Cas9 molecule and gRNA molecule described herein. Exemplary targets include, e.g., genes associated with control of the gamma-globin genes. In an embodiment, the target is a nondeletional HPFH region.

Fetal hemoglobin (also hemoglobin F or HbF or a2y2) is a tetramer of two adult alpha-globin polypeptides and two fetal beta-like gamma-globin polypeptides. HbF is the main oxygen transport protein in the human fetus during the last seven months of development in the uterus and in the newborn until roughly 6 months old. Functionally, fetal hemoglobin differs most from adult hemoglobin in that it is able to bind oxygen with greater affinity than the adult form, giving the developing fetus better access to oxygen from the mother's bloodstream.

In newborns, fetal hemoglobin is nearly completely replaced by adult hemoglobin by approximately 6 months postnatally. In adults, fetal hemoglobin production can be reactivated pharmacologically, which is useful in the treatment of diseases such as hemoglobinopathies. For example, in certain patients with hemoglobinopathies, higher levels of gamma-globin expression can partially compensate for defective or impaired beta-globin gene production, which can ameliorate the clinical severity in these diseases. Increased HbF levels or F-cell (HbF containing erythrocyte) numbers can ameliorate the disease severity of hemoglobinopathies, e.g., beta-thalassemia major and sickle cell anemia.

### Sickle Cell Diseases

Sickle cell disease is a group of disorders that affects hemoglobin. People with this disorder have atypical hemoglobin molecules (hemoglobin S), which can distort red blood cells into a sickle, or crescent, shape. Characteristic features of this disorder include a low number of red blood cells (anemia), repeated infections, and periodic episodes of pain.

Mutations in the HBB gene cause sickle cell disease. The HBB gene provides instructions for making beta-globin. Various versions of beta-globin result from different mutations in the HBB gene. One particular HBB gene mutation produces an abnormal version of beta-globin known as hemoglobin S (HbS). Other mutations in the HBB gene lead to additional abnormal versions of beta-globin such as hemoglobin C (HbC) and hemoglobin E (HbE). HBB gene mutations can also result in an unusually low level of beta-globin, i.e., beta thalassemia.

In people with sickle cell disease, at least one of the beta-globin subunits in hemoglobin is replaced with hemoglobin S. In sickle cell anemia, which is a common form of sickle cell disease, hemoglobin S replaces both beta-globin subunits in hemoglobin. In other types of sickle cell disease, just one beta-globin subunit in hemoglobin is replaced with hemoglobin S. The other beta-globin subunit is replaced with a different abnormal variant, such as hemoglobin C. For example, people with sickle-hemoglobin C (HbSC) disease have hemoglobin molecules with hemoglobin S and hemoglobin C instead of beta-globin. If mutations that produce hemoglobin S and beta thalassemia occur together, individuals have hemoglobin S-beta thalassemia (HbSBetaThal) disease.

### Beta Thalassemia

Beta thalassemia is a blood disorder that reduces the production of hemoglobin. In people with beta thalassemia, low levels of hemoglobin lead to a lack of oxygen in many parts of the body. Affected individuals also have a shortage of red blood cells (anemia), which can cause pale skin, weakness, fatigue, and more serious complications. People with beta thalassemia are at an increased risk of developing abnormal blood clots.

Beta thalassemia is classified into two types depending on the severity of symptoms: thalassemia major (also known as Cooley's anemia) and thalassemia intermedia. Of the two types, thalassemia major is more severe.

Mutations in the HBB gene cause beta thalassemia. The HBB gene provides instructions for making beta-globin. Some mutations in the HBB gene prevent the production of any beta-globin. The absence of beta-globin is referred to as beta-zero (B°) thalassemia. Other HBB gene mutations allow some beta-globin to be produced but in reduced amounts, i.e., beta-plus (B*) thalassemia. People with both types have been diagnosed with thalassemia major and thalassemia intermedia.

In an embodiment, a Cas9 molecule/gRNA molecule complex targeting a first gene or locus is used to treat a disorder characterized by a second gene, e.g., a mutation in a second gene. By way of example, targeting of the first gene, e.g., by editing or payload delivery, can compensate for, or inhibit further damage from, the effect of a second gene, e.g., a mutant second gene. In an embodiment the allele(s) of the first gene carried by the subject is not causative of the disorder.

In one aspect, the invention relates to the treatment of a mammal, e.g., a human, in need of increased fetal hemoglobin (HbF).

In one aspect, the invention relates to the treatment of a mammal, e.g., a human, that has been diagnosed with, or is at risk of developing, a hemoglobinopathy.

In one aspect, the hemoglobinopathy is a β-hemoglobinopathy. In one aspect, the hemoglobinopathy is sickle cell disease. In one aspect, the hemoglobinopathy is beta thalassemia.

### Methods of Treatment of Hemoglobinopathies

In another aspect the invention provides methods of treatment. In aspects, the gRNA molecules, CRISPR systems and/or cells of the invention are used to treat a patient in need thereof. In aspects, the patient is a mammal, e.g., a human. In aspects, the patient has a hemoglobinopathy. In embodiments, the patient has sickle cell disease. In embodiments, the patient has beta thalassemia.

In one aspect, the method of treatment comprises administering to a mammal, e.g., a human, one or more gRNA molecules, e.g., one or more gRNA molecules comprising a targeting domain described in Table 1, and one or more cas9 molecules described herein.

In one aspect, the method of treatment comprises administering to a mammal a cell population, wherein the cell population is a cell population from a mammal, e.g., a human, that has been administered one or more gRNA molecules, e.g., one or more gRNA molecules comprising a targeting domain described in Table 1, and one or more cas9 molecules described herein, e.g., a CRISPR system as described herein. In one embodiment, the administration of the one or more gRNA molecules or CRISPR systems to the cell is accomplished in vivo. In one embodiment the administration of the one or more gRNA molecules or CRISPR systems to the cell is accomplished ex vivo.

In one aspect, the method of treatment comprises administering to the mammal, e.g., the human, an effective amount of a cell population comprising cells which comprise or at one time comprised one or more gRNA molecules, e.g., one or more gRNA molecules comprising a targeting domain described in Table 1, and one or more cas9 molecules described herein, or the progeny of said cells. In one embodiment, the cells are allogeneic to the mammal. In one embodiment, the cells are autologous to the mammal. In one embodiment the cells are harvested from the mammal, manipulated ex vivo, and returned to the mammal.

In aspects, the cells comprising or which at one time comprised one or more gRNA molecules, e.g., one or more gRNA molecules comprising a targeting domain described in Table 1, and one or more cas9 molecules described herein, or the progeny of said cells, comprise stem cells or progenitor cells. In one aspect, the stem cells are hematopoietic stem cells. In one aspect, the progenitor cells are hematopoietic progenitor cells. In one aspect, the cells comprise both hematopoietic stem cells and hematopoietic progenitor cells, e.g., are HSPCs. In one aspect, the cells comprise, e.g., consist of, CD34+ cells. In one aspect the cells are substantially free of CD34− cells. In one aspect, the cells comprise, e.g., consist of, CD34+/CD90+ stem cells. In one aspect, the cells comprise, e.g., consist of, CD34+/CD90− cells. In an aspect, the cells are a population comprising one or more of the cell types described above or described herein.

In one embodiment, the disclosure provides a method for treating a hemoglobinopathy, e.g., sickle cell disease or beta-thalassemia, or a method for increasing fetal hemoglobin expression in a mammal, e.g., a human, in need thereof, the method comprising:
a) providing, e.g., harvesting or isolating, a population of HSPCs (e.g., CD34+ cells) from a mammal;
b) providing said cells ex vivo, e.g., in a cell culture medium, optionally in the presence of an effective amount of a composition comprising at least one stem cell expander, whereby said population of HSPCs (e.g., CD34+ cells) expands to a greater degree than an untreated population;
c) contacting the population of HSPCs (e.g., CD34+ cells) with an effective amount of: a composition comprising at least one gRNA molecule comprising a targeting domain described herein, e.g., a targeting domain described in Table 1, or a nucleic acid encoding said gRNA molecule, and at least one cas9 molecule, e.g., described herein, or a nucleic acid encoding said cas9 molecule, e.g., one or more RNPs as described herein, e.g., with a CRISPR system described herein;
d) causing at least one modification in at least a portion of the cells of the population (e.g., at least a portion of the HSPCs, e.g., CD34+ cells, of the population), whereby, e.g., when said HSPCs are differentiated into cells of an erythroid lineage, e.g., red blood cells, fetal hemoglobin expression is increased, e.g., relative to cells not contacted according to step c); and
f) returning a population of cells comprising said modified HSPCs (e.g., CD34+ cells) to the mammal.

In an aspect, the HSPCs are allogeneic to the mammal to which they are returned. In an aspect, the HSPCs are autologous to the mammal to which they are returned. In aspects, the HSPCs are isolated from bone marrow. In aspects, the HSPCs are isolated from peripheral blood, e.g., mobilized peripheral blood. In aspects, the mobilized peripheral blood is isolated from a subject who has been administered a G-CSF. In aspects, the mobilized peripheral blood is isolated from a subject who has been administered a mobilization agent other than G-CSF, for example, Plerixafor® (AMD3100). In other aspects, the mobilized peripheral blood is isolated from a subject who has been administered a combination of G-CSF and Plerixafor® (AMD3100)). In aspects, the HSPCs are isolated fromumbilical cord blood. In embodiments, the cells are derived from a hemoglobinopathy patient, for example a patient with sickle cell disease or a patient with a thalassemia, e.g., beta-thalassemia.

In further embodiments of the method, the method further comprises, after providing a population of HSPCs (e.g., CD34+ cells), e.g., from a source described above, the step of enriching the population of cells for HSPCs (e.g., CD34+ cells). In embodiments of the method, after said enriching, the population of cells, e.g., HSPCs, is substantially free of CD34− cells.

In embodiments, the population of cells which is returned to the mammal includes at least 70% viable cells. In embodiments, the population of cells which is returned to the mammal includes at least 75% viable cells. In embodiments, the population of cells which is returned to the mammal includes at least 80% viable cells. In embodiments, the population of cells which is returned to the mammal includes at least 85% viable cells. In embodiments, the population of cells which is returned to the mammal includes at least 90% viable cells. In embodiments, the population of cells which is returned to the mammal includes at least 95% viable cells. In embodiments, the population of cells which is returned to the mammal includes at least 99% viable cells. Viability can be determined by staining a representative portion of the population of cells for a cell viability marker, e.g., as known in the art.

In another embodiment, the disclosure provides a method for treating a hemoglobinopathy, e.g., sickle cell disease or beta-thalassemia, or a method for increasing fetal hemoglobin expression in a mammal, e.g., a human, in need thereof, the method comprising the steps of:
a) providing, e.g., harvesting or isolating, a population of HSPCs (e.g., CD34+ cells) of a mammal, e.g., from the bone marrow of a mammal;
b) isolating the CD34+ cells from the population of cells of step a);
c) providing said CD34+ cells ex vivo, and culturing said cells, e.g., in a cell culture medium, in the presence of an effective amount of a composition comprising at least one stem cell expander, e.g., (S)-2-(6-(2-(1H-indol-3-yl)ethylamino)-2-(5-fluoropyridin-3-yl)-9H-purin-9-yl)propan-1-ol, e.g., (S)-2-(6-(2-(1H-indol-3-yl)ethylamino)-2-(5-fluoropyridin-3-yl)-9H-purin-9-yl)propan-1-ol at a concentration of about 0.5 to about 0.75 micromolar, whereby said population of CD34+ cells expands to a greater degree than an untreated population;
d) introducing into the cells of the population CD34+ cells an effective amount of: a composition comprising a Cas9 molecule, e.g., as described herein, and a gRNA molecule, e.g., as described herein, e.g., optionally where the Cas9 molecule and the gRNA molecule are in the form of an RNP, e.g., as described herein, and optionally where said introduction is by electroporation, e.g., as described herein, of said RNP into said cells;
e) causing at least one genetic modification in at least a portion of the cells of the population (e.g., at least a portion of the HSPCs, e.g., CD34+ cells, of the population), whereby an indel, e.g., as described herein, is created at or near the genomic site complementary to the targeting domain of the gRNA introduced in step d);
f) optionally, additionally culturing said cells after said introducing, e.g., in a cell culture medium, in the presence of an effective amount of a composition comprising at least one stem cell expander, e.g., (S)-2-(6-(2-(1H-indol-3-yl)ethylamino)-2-(5-fluoropyridin-3-yl)-9H-purin-9-yl)propan-1-ol, e.g., (S)-2-(6-(2-(1H-indol-3-yl)ethylamino)-2-(5-fluoropyridin-3-yl)-9H-purin-9-yl)propan-1-ol at a concentration of about 0.5 to about 0.75 micromolar, such that the cells expand at least 2-fold, e.g., at least 4-fold, e.g., at least 5-fold;
g) cryopreserving said cells; and
h) returning the cells to the mammal, wherein, the cells returned to the mammal comprise cells that 1) maintain the ability to differentiate into cells of the erythroid lineage, e.g., red blood cells; 2) when differentiated into red blood cells, produce an increased level of fetal hemoglobin, e.g., relative to cells unmodified by the gRNA of step e), e.g., produce at least 6 picograms fetal hemoglobin per cell.

In an aspect, the HSPCs are allogeneic to the mammal to which they are returned. In an aspect, the HSPCs are autologous to the mammal to which they are returned. In aspects, the HSPCs are isolated from bone marrow. In aspects, the HSPCs are isolated from peripheral blood, e.g., mobilized peripheral blood. In aspects, the mobilized peripheral blood is isolated from a subject who has been administered a G-CSF. In aspects, the mobilized peripheral blood is isolated from a subject who has been administered a mobilization agent other than G-CSF, for example, Plerixafor® (AMD3100). In other aspects, the mobilized peripheral blood is isolated from a subject who has been administered a combination of G-CSF and Plerixafor® (AMD3100)). In aspects, the HSPCs are isolated fromumbilical cord blood. In embodiments, the cells are derived from a hemoglobinopathy patient, for example a patient with sickle cell disease or a patient with a thalassemia, e.g., beta-thalassemia.

In embodiments of the method above, the recited step b) results in a population of cells which is substantially free of CD34− cells.

In further embodiments of the method, the method further comprises, after providing a population of HSPCs (e.g., CD34+ cells), e.g., from a source described above, the population of cells is enriched for HSPCs (e.g., CD34+ cells).

In a further embodiments of these methods, the population of modified HSPCs (e.g., CD34+ stem cells) having the ability to differentiate with increased fetal hemoglobin expression is cryopreserved and stored prior to being reintroduced into the mammal. In embodiments, the cryopreserved population of HSPCs having the ability to differentiate into cells of the erythroid lineage, e.g., red blood cells, and/or when differentiated into cells of the erythroid lineage, e.g., red blood cells, produce an increased level of fetal hemoglobin is thawed and then reintroduced into the mammal. In a further embodiment of these methods, the method comprises chemotherapy and/or radiation therapy to remove or reduce the endogenous hematopoietic progenitor or stem cells in the mammal. In a further embodiment of these methods, the method does not comprise a step of chemotherapy and/or radiation therapy to remove or reduce the endogenous hematopoietic progenitor or stem cells in the mammal. In a further embodiment of these methods, the method comprises a chemotherapy and/or radiation therapy to reduce partially (e.g., partial lymphodepletion) the endogenous hematopoietic progenitor or stem cells in the mammal. In embodiments the patient is treated with a fully lymphodepleting dose of busulfan prior to reintroduction of the modified HSPCs to the mammal. In embodiments, the patient is treated with a partially lymphodepleting dose of busulfan prior to reintroduction of the modified HSPCs to the mammal. In embodiments, the patient is treated with HSC-targeted antibody-drug conjugates for conditioning. In embodiments, such HSC-targeted antibody-drug conjugates can be found in WO2018071871, the contents of which are incorporated herein by reference.

In embodiments, the cells are contacted with RNP comprising a Cas9 molecule, e.g., as described herein, complexed with a gRNA to ZNF644, e.g., as described herein (e.g., comprising a targeting domain listed in Table 1-Table 3.

In embodiments, the stem cell expander is Compound 1. In embodiments, the stem cell expander is Compound 2. In embodiments, the stem cell expander is Compound 3. In embodiments, the stem cell expander is (S)-2-(6-(2-(1H-indol-3-yl)ethylamino)-2-(5-fluoropyridin-3-yl)-9H-purin-9-yl)propan-1-ol. In embodiments, the stem cell expander is (S)-2-(6-(2-(1H-indol-3-yl)ethylamino)-2-(5-fluoropyridin-3-yl)-9H-purin-9-yl)propan-1-ol and is present at a concentration of 2-0.1 micromolar, e.g., 1-0.25 micromolar, e.g., 0.75-0.5 micromolar. In embodiments, the stem cell expander is a molecule described in WO2010/059401 (e.g., the molecule described in Example 1 of WO2010/059401).

In embodiments, the cells, e.g., HSPCs, e.g., as described herein, are cultured ex vivo for a period of about 1 hour to about 15 days, e.g., a period of about 12 hours to about 12 days, e.g., a period of about 12 hours to 4 days, e.g., a period of about 1 day to about 4 days, e.g., a period of about 1 day to about 2 days, e.g., a period of about 1 day or a period of about 2 days, prior to the step of contacting the cells with a CRISPR system, e.g., described herein. In embodiments, said culturing prior to said contacting step is in a composition (e.g., a cell culture medium) comprising a stem cell expander, e.g., described herein, e.g., (S)-2-(6-(2-(1H-indol-3-yl)ethylamino)-2-(5-fluoropyridin-3-yl)-9H-purin-9-yl)propan-1-ol, e.g., (S)-2-(6-(2-(1H-indol-3-yl)ethylamino)-2-(5-fluoropyridin-3-yl)-9H-purin-9-yl)propan-1-ol at a concentration of about 0.25 uM to about 1 uM, e.g., (S)-2-(6-(2-(1H-indol-3-yl)ethylamino)-2-(5-fluoropyridin-3-yl)-9H-purin-9-yl)propan-1-ol at a concentration of about 0.75-0.5 micromolar. In embodiments, the cells are cultured ex vivo for a period of no more than about 1 day, e.g., no more than about 18, 17, 16, 15, 14, 13, 12, 11, 10, 9, 8, 7, 6, 5, 4, 3, 2, or 1 hour(s) after the step of contacting the cells with a CRISPR system, e.g., described herein, e.g., in a cell culture medium which comprises a stem cell expander, e.g., described herein, e.g., (S)-2-(6-(2-(1H-indol-3-yl)ethylamino)-2-(5-fluoropyridin-3-yl)-9H-purin-9-yl)propan-1-ol, e.g., (S)-2-(6-(2-(1H-indol-3-yl)ethylamino)-2-(5-fluoropyridin-3-yl)-9H-purin-9-yl)propan-1-ol at a concentration of about 0.25 uM to about 1 uM, e.g., (S)-2-(6-(2-(1H-indol-3-yl)ethylamino)-2-(5-fluoropyridin-3-yl)-9H-purin-9-yl)propan-1-ol at a concentration of about 0.75-0.5 micromolar. In other embodiments, the cells are cultured ex vivo for a period of about 1 hour to about 15 days, e.g., a period of about 12 hours to about 10 days, e.g., a period of about 1 day to about 10 days, e.g., a period of about 1 day to about 5 days, e.g., a period of about 1 day to about 4 days, e.g., a period of about 2 days to about 4 days, e.g., a period of about 2 days, about 3 days or about 4 days, after the step of contacting the cells with a CRISPR system, e.g., described herein, in a cell culture medium, e.g., which comprises a stem cell expander, e.g., described herein, e.g., (S)-2-(6-(2-(1H-indol-3-yl)ethylamino)-2-(5-fluoropyridin-3-yl)-9H-purin-9-yl)propan-1-ol, e.g., (S)-2-(6-(2-(1H-indol-3-yl)ethylamino)-2-(5-fluoropyridin-3-yl)-9H-purin-9-yl)propan-1-ol at a concentration of about 0.25 uM to about 1 uM, e.g., (S)-2-(6-(2-(1H-indol-3-yl)ethylamino)-2-(5-fluoropyridin-3-yl)-9H-purin-9-yl)propan-1-ol at a concentration of about 0.75-0.5 micromolar. In embodiments, the cells are cultured ex vivo (e.g., cultured prior to said contacting step and/or cultured after said contacting step) for a period of about 1 hour to about 20 days, e.g., a period of about 6-12 days, e.g., a period of about 6, about 7, about 8, about 9, about 10, about 11, or about 12 days.

In embodiments, the population of cells comprising the modified HSPCs returned to the mammal comprises at least about 1 million cells (e.g., at least about 1 million CD34+ cells) per kg. In embodiments, the population of cells comprising the modified HSPCs returned to the mammal comprises at least about 2 million cells (e.g., at least about 2 million CD34+ cells) per kg. In embodiments, the population of cells comprising the modified HSPCs returned to the mammal comprises at least about 3 million cells (e.g., at least about 3 million CD34+ cells) per kg. In embodiments, the population of cells comprising the modified HSPCs returned to the mammal comprises at least about 4 million cells (e.g., at least about 4 million CD34+ cells) per kg. In embodiments, the population of cells comprising the modified HSPCs returned to the mammal comprises at least about 5 million cells (e.g., at least about 5 million CD34+ cells) per kg. In embodiments, the population of cells comprising the modified HSPCs returned to the mammal comprises at least about 6 million cells (e.g., at least about 6 million CD34+ cells) per kg. In embodiments, the population of cells comprising the modified HSPCs returned to the mammal comprises at least 1 million cells (e.g., at least 1 million CD34+ cells) per kg. In embodiments, the population of cells comprising the modified HSPCs returned to the mammal comprises at least 2 million cells (e.g., at least 2 million CD34+ cells) per kg. In embodiments, the population of cells comprising the modified HSPCs returned to the mammal comprises at least 3 million cells (e.g., at least 3 million CD34+ cells) per kg. In embodiments, the population of cells comprising the modified HSPCs returned to the mammal comprises at least 4 million cells (e.g., at least 4 million CD34+ cells) per kg. In embodiments, the population of cells comprising the modified HSPCs returned to the mammal comprises at least 5 million cells (e.g., at least 5 million CD34+ cells) per kg. In embodiments, the population of cells comprising the modified HSPCs returned to the mammal comprises at least 6 million cells (e.g., at least 6 million CD34+ cells) per kg. In embodiments, the population of cells comprising the modified HSPCs returned to the mammal comprises about 1 million cells (e.g., about 1 million CD34+ cells) per kg. In embodiments, the population of cells comprising the modified HSPCs returned to the mammal comprises about 2 million cells (e.g., about 2 million CD34+ cells) per kg. In embodiments, the population of cells comprising the modified HSPCs returned to the mammal comprises about 3 million cells (e.g., about 3 million CD34+ cells) per kg. In embodiments, the population of cells comprising the modified HSPCs returned to the mammal comprises about 4 million cells (e.g., about 4 million CD34+ cells) per kg. In embodiments, the population of cells comprising the modified HSPCs returned to the mammal comprises about 5 million cells (e.g., about 5 million CD34+ cells) per kg. In embodiments, the population of cells comprising the modified HSPCs returned to the mammal comprises about 6 million cells (e.g., about 6 million CD34+ cells) per kg. In embodiments, the population of cells comprising the modified HSPCs returned to the mammal comprises about 2×10⁶ cells (e.g., about 2×10⁶ CD34+ cells) per kg body weight of the patient. In embodiments, the population of cells comprising the modified HSPCs returned to the mammal comprises at least 2×10⁶ cells (e.g., about 2×10⁶ CD34+ cells) per kg body weight of the patient. In embodiments, the population of cells comprising the modified HSPCs returned to the mammal comprises between 2×10⁶ cells (e.g., about 2×10⁶ CD34+ cells) per kg body weight of the patient and 10×10⁶ cells (e.g., about 2×10⁶ CD34+ cells) per kg body weight of the patient. In embodiments, the cells comprising the modified cells are infused into the patient. In embodiments, before the cells comprising the modified HSPCs are infused into the patient, the patient is treated with a lymphodepleting therapy, for example, is treated with busulphan, for example is treated with a full lymphodepleting busulphan regimen, or for example is treated with a reduced intensity busulphan lymphodepleting regimen.

In embodiments, any of the methods described above results in the patient having at least 80% of its circulating CD34+ cells comprising an indel at or near the genomic site complementary to the targeting domain of the gRNA molecule used in the method, e.g., as measured at least 15 days, e.g., at least 20, at least 30, at least 40 at least 50 or at least 60 days after reintroduction of the cells into the mammal. Without being bound by theory, it has surprisingly been discovered herein that indels and indel patterns (including large deletions) observed when gene editing systems, e.g., CRISPR systems, e.g., CRISPR systems comprising a gRNA molecule targeting the ZNF644 gene region, e.g., as described herein, are introduced into HSPCs, and those cells are transplanted into organisms, certain gRNAs produce cells comprising indels and indel patterns (including large indels) that remain detectible in the edited cell population and its progeny, in the organism, and persist for more than 8 weeks, 12 weeks, 16 weeks or 20 weeks. Without being bound by theory, a cell population comprising an indel pattern or particular indel (including large deletion) that persists within a detectible cell population, for example, longer than 16 weeks or longer than 20 weeks after introduction into an organism (e.g., a patient), could be beneficial to producing a longer-term amelioration of a disease or condition, e.g. described herein (e.g., a hemoglobinopathy, e.g., sickle cell disease or a thalassemia) than cells (or their progeny) that upon introduction into an organism or patient lose one or more indels (including large deletions). In embodiments, the persisting indel or indel pattern is associated with upregulated fetal hemoglobin (e.g., in erythroid progeny of said cells). Thus, in embodiments, the present disclosure provides populations of cells, e.g., HSPCs, e.g., as described herein, which comprise one or more indels (including large deletions) which persist (e.g., remain detectible, e.g., in a cell population or its progeny) in the blood and/or bone marrow) for more than 8 weeks, more than 12 weeks, more than 16 weeks or more than 20 weeks after introduction into an organism, e.g., patient.

In embodiments, any of the methods described above results in the patient having at least 20% of its bone marrow CD34+ cells comprising an indel at or near the genomic site complementary to the targeting domain of the gRNA molecule used in the method, e.g., as measured at least 15 days, e.g., at least 20, at least 30, at least 40 at least 50 or at least 60 days after reintroduction of the cells into the mammal.

In embodiments, the HSPCs that are reintroduced into the mammal are able to differentiate in vivo into cells of the erythroid lineage, e.g., red blood cells, and said differentiated cells exhibit increased fetal hemoglobin levels, e.g., produce at least 6 picograms fetal hemoglobin per cell, e.g., at least 7 picograms fetal hemoglobin per cell, at least 8 picograms fetal hemoglobin per cell, at least 9 picograms fetal hemoglobin per cell, at least 10 picograms fetal hemoglobin per cell, e.g., between about 9 and about 10 picograms fetal hemoglobin per cell, e.g., such that the hemoglobinopathy is treated the mammal.

It will be understood that when a cell is characterized as having increased fetal hemoglobin, that includes embodiments in which a progeny, e.g., a differentiated progeny, of that cell exhibits increased fetal hemoglobin. For example, in the methods described herein, the altered or modified CD34+ cell (or cell population) may not express increased fetal hemoglobin, but when differentiated into cells of erythroid lineage, e.g., red blood cells, the cells express increased fetal hemoglobin, e.g., increased fetal hemoglobin relative to an unmodified or unaltered cell under similar conditions.

### XI. Culture Methods and Methods of Manufacturing Cells

The disclosure provides methods of culturing cells, e.g., HSPCs, e.g., hematopoietic stem cells, e.g., CD34+ cells modified, or to be modified, with the gRNA molecules described herein.

### DNA Repair Pathway Inhibitors

Without being bound by theory, it is believed that the pattern of indels produced by a given gRNA molecule at a particular target sequence is a product of each of the active DNA repair mechanisms within the cell (e.g., non-homologous end joining, microhomology-mediated end joining, etc.). Without being bound by theory, it is believed that a particularly favorable indel may be selected for or enriched for by contacting the cells to be edited with an inhibitor of a DNA repair pathway that does not produce the desired indel. Thus, the gRNA molecules, CRISPR systems, methods and other aspects of the invention may be performed in combination with such inhibitors. Examples of such inhibitors include those described in, e.g., WO2014/130955, the contents of which are hereby incorporated by reference in their entirety. In embodiment, the inhibitor is a DNAPKc inhibitor, e.g., NU7441.

### Stem Cell Expanders

In one aspect the invention relates to culturing the cells, e.g., HSPCs, e.g., CD34+ cells modified, or to be modified, with the gRNA molecules described herein, with one or more agents that result in an increased expansion rate, increased expansion level, or increased engraftment relative to cells not treated with the agent. Such agents are referred to herein as stem cell expanders.

In an aspect, the one or more agents that result in an increased expansion rate or increased expansion level, relative to cells not treated with the agent, e.g., the stem cell expander, comprises an agent that is an antagonist of the aryl hydrocarbon receptor (AHR) pathway. In aspects, the stem cell expander is a compound disclosed in WO2013/110198 or a compound disclosed in WO2010/059401, the contents of which are incorporated by reference in their entirety.

In one aspect, the one or more agents that result in an increased expansion rate or increased expansion level, relative to cells not treated with the agent, is a pyrimido[4,5-b]indole derivative, e.g., as disclosed in WO2013/110198, the contents of which are hereby incorporated by reference in their entirety. In one embodiment the agent is compound 1 ((1r,4r)-N¹-(2-benzyl-7-(2-methyl-2H-tetrazol-5-yl)-9H-pyrimido[4,5-b]indol-4-yl)cyclohexane-1,4-diamine):

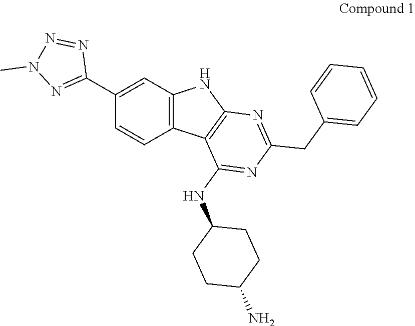 [SMILES: Cn1nnc(-c2ccc3c(c2)[nH]c2nc(Cc4ccccc4)nc(N[C@H]4CC[C@H](N)CC4)c23)n1]

In another aspect, the agent is Compound 2 (methyl 4-(3-piperidin-1-ylpropylamino)-9H-pyrimido[4,5-b]indole-7-carboxylate):

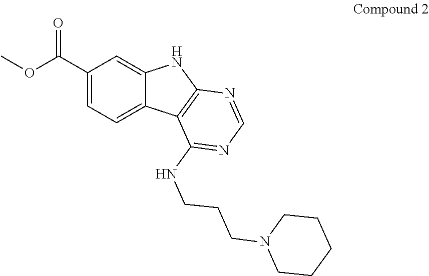 [SMILES: COC(=O)c1ccc2c(c1)[nH]c1ncnc(NCCCN3CCCCC3)c12]

In another aspect, the one or more agents that result in an increased expansion rate or increased expansion level, relative to cells not treated with the agent, is an agent disclosed in WO2010/059401, the contents of which are hereby incorporated by reference in their entirety.

In one embodiment, the stem cell expander is compound 3: 4-(2-(2-(benzo[b]thiophen-3-yl)-9-isopropyl-9H-purin-6-ylamino)ethyl)phenol, i.e., is the compound from example 1 of WO2010/059401, having the following structure:

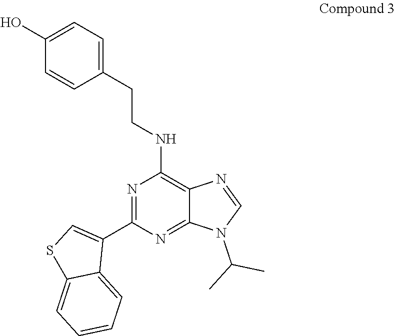 [SMILES: CC(C)n1cnc2c(NCCc3ccc(O)cc3)nc(-c3csc4ccccc34)nc21]

In another aspect, the stem cell expander is (S)-2-(6-(2-(1H-indol-3-yl)ethylamino)-2-(5-fluoropyridin-3-yl)-9H-purin-9-yl)propan-1-ol ((S)-2-(6-(2-(1H-indol-3-yl)ethylamino)-2-(5-fluoropyridin-3-yl)-9H-purin-9-yl)propan-1-ol, i.e., is the compound 157S according to WO2010/059401), having the following structure:

### (S)-2-(6-(2-(1H-indol-3-yl)ethylamino)-2-(5-fluoropyridin-3-yl)-9H-purin-9-yl)propan-ol

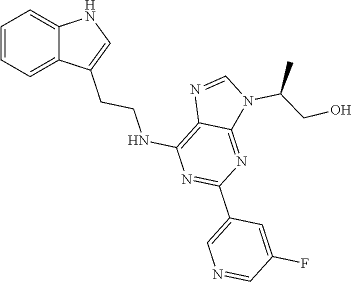 [SMILES: C[C@@H](CO)n1cnc2c(NCCc3c[nH]c4ccccc34)nc(-c3cncc(F)c3)nc21]

In embodiments the population of HSPCs is contacted with the stem cell expander, e.g., compound 1, compound 2, compound 3, (S)-2-(6-(2-(1H-indol-3-yl)ethylamino)-2-(5-fluoropyridin-3-yl)-9H-purin-9-yl)propan-1-ol, or combinations thereof (e.g., a combination of compound 1 and (S)-2-(6-(2-(1H-indol-3-yl)ethylamino)-2-(5-fluoropyridin-3-yl)-9H-purin-9-yl)propan-1-ol) before introduction of the CRISPR system (e.g., gRNA molecule and/or Cas9 molecule of the invention) to said HSPCs. In embodiments, the population of HSPCs is contacted with the stem cell expander, e.g., compound 1, compound 2, compound 3, (S)-2-(6-(2-(1H-indol-3-yl)ethylamino)-2-(5-fluoropyridin-3-yl)-9H-purin-9-yl)propan-1-ol, or combinations thereof (e.g., a combination of compound 1 and (S)-2-(6-(2-(1H-indol-3-yl)ethylamino)-2-(5-fluoropyridin-3-yl)-9H-purin-9-yl)propan-1-ol), after introduction of the CRISPR system (e.g., gRNA molecule and/or Cas9 molecule of the invention) to said HSPCs. In embodiments, the population of HSPCs is contacted with the stem cell expander, e.g., compound 1, compound 2, compound 3, (S)-2-(6-(2-(1H-indol-3-yl)ethylamino)-2-(5-fluoropyridin-3-yl)-9H-purin-9-yl)propan-1-ol, or combinations thereof (e.g., a combination of compound 1 and (S)-2-(6-(2-(1H-indol-3-yl)ethylamino)-2-(5-fluoropyridin-3-yl)-9H-purin-9-yl)propan-1-ol), both before and after introduction of the CRISPR system (e.g., gRNA molecule and/or Cas9 molecule of the invention) to said HSPCs.

In embodiments, the stem cell expander is present in an effective amount to increase the expansion level of the HSPCs, relative to HSPCs in the same media but for the absence of the stem cell expander. In embodiments, the stem cell expander is present at a concentration ranging from about 0.01 to about 10 uM, e.g., from about 0.1 uM to about 1 uM. In embodiments, the stem cell expander is present in the cell culture medium at a concentration of about 1 uM, about 950 nM, about 900 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 50 nM, about 25 nM, or about 10 nM. In embodiments, the stem cell expander is present at a concentration ranging from about 500 nM to about 750 nM.

In embodiments, the stem cell expander is (S)-2-(6-(2-(1H-indol-3-yl)ethylamino)-2-(5-fluoropyridin-3-yl)-9H-purin-9-yl)propan-1-ol, which is present in the cell culture medium at a concentration ranging from about 0.01 to about 10 micromolar (uM). In embodiments, the stem cell expander is (S)-2-(6-(2-(1H-indol-3-yl)ethylamino)-2-(5-fluoropyridin-3-yl)-9H-purin-9-yl)propan-1-ol, which is present in the cell culture medium at a concentration ranging from about 0.1 to about 1 micromolar (uM). In embodiments, the stem cell expander is (S)-2-(6-(2-(1H-indol-3-yl)ethylamino)-2-(5-fluoropyridin-3-yl)-9H-purin-9-yl)propan-1-ol, which is present in the cell culture medium at a concentration of about 0.75 micromolar (uM). In embodiments, the stem cell expander is (S)-2-(6-(2-(1H-indol-3-yl)ethylamino)-2-(5-fluoropyridin-3-yl)-9H-purin-9-yl)propan-1-ol, which is present in the cell culture medium at a concentration of about 0.5 micromolar (uM). In embodiments of any of the foregoing, the cell culture medium additionally comprises compound 1.

In embodiments, the stem cell expander is a mixture of compound 1 and (S)-2-(6-(2-(1H-indol-3-yl)ethylamino)-2-(5-fluoropyridin-3-yl)-9H-purin-9-yl)propan-1-ol.

In embodiments, the cells of the invention are contacted with one or more stem cell expander molecules for a sufficient time and in a sufficient amount to cause a 2 to 10,000-fold expansion of CD34+ cells, e.g., a 2-1000-fold expansion of CD34+ cells, e.g., a 2-100-fold expansion of CD34+ cells, e.g., a 20-200-fold expansion of CD34+ cells. As described herein, the contacting with the one or more stem cell expanders may be before the cells are contacted with a CRISPR system, e.g., as described herein, after the cells are contacted with a CRISPR system, e.g., as described herein, or a combination thereof. In an embodiment, the cells are contacted with one or more stem cell expander molecules, e.g., (S)-2-(6-(2-(1H-indol-3-yl)ethylamino)-2-(5-fluoropyridin-3-yl)-9H-purin-9-yl)propan-1-ol, for a sufficient time and in a sufficient amount to cause at least a 2-fold expansion of CD34+ cells, e.g., CD34+ cells comprising an indel at or near the target site having complementarity to the targeting domain of the gRNA of the CRISPR/Cas9 system introduced into said cell. In an embodiment, the cells are contacted with one or more stem cell expander molecules, e.g., (S)-2-(6-(2-(1H-indol-3-yl)ethylamino)-2-(5-fluoropyridin-3-yl)-9H-purin-9-yl)propan-1-ol, for a sufficient time and in a sufficient amount to cause at least a 4-fold expansion of CD34+ cells, e.g., CD34+ cells comprising an indel at or near the target site having complementarity to the targeting domain of the gRNA of the CRISPR/Cas9 system introduced into said cell. In an embodiment, the cells are contacted with one or more stem cell expander molecules, e.g., (S)-2-(6-(2-(1H-indol-3-yl)ethylamino)-2-(5-fluoropyridin-3-yl)-9H-purin-9-yl)propan-1-ol, for a sufficient time and in a sufficient amount to cause at least a 5-fold expansion of CD34+ cells, e.g., CD34+ cells comprising an indel at or near the target site having complementarity to the targeting domain of the gRNA of the CRISPR/Cas9 system introduced into said cell. In an embodiment, the cells are contacted with one or more stem cell expander molecules for a sufficient time and in a sufficient amount to cause at least a 10-fold expansion of CD34+ cells. In an embodiment, the cells are contacted with one or more stem cell expander molecules for a sufficient time and in a sufficient amount to cause at least a 20-fold expansion of CD34+ cells. In an embodiment, the cells are contacted with one or more stem cell expander molecules for a sufficient time and in a sufficient amount to cause at least a 30-fold expansion of CD34+ cells. In an embodiment, the cells are contacted with one or more stem cell expander molecules for a sufficient time and in a sufficient amount to cause at least a 40-fold expansion of CD34+ cells. In an embodiment, the cells are contacted with one or more stem cell expander molecules for a sufficient time and in a sufficient amount to cause at least a 50-fold expansion of CD34+ cells. In an embodiment, the cells are contacted with one or more stem cell expander molecules for a sufficient time and in a sufficient amount to cause at least a 60-fold expansion of CD34+ cells. In embodiments, the cells are contacted with the one or more stem cell expanders for a period of about 1-60 days, e.g., about 1-50 days, e.g., about 1-40 days, e.g., about 1-30 days, e.g., 1-20 days, e.g., about 1-10 days, e.g., about 7 days, e.g., about 1-5 days, e.g., about 2-5 days, e.g., about 2-4 days, e.g., about 2 days or, e.g., about 4 days.

In embodiments, the cells, e.g., HSPCs, e.g., as described herein, are cultured ex vivo for a period of about 1 hour to about 10 days, e.g., a period of about 12 hours to about 5 days, e.g., a period of about 12 hours to 4 days, e.g., a period of about 1 day to about 4 days, e.g., a period of about 1 day to about 2 days, e.g., a period of about 1 day or a period of about 2 days, prior to the step of contacting the cells with a CRISPR system, e.g., described herein. In embodiments, said culturing prior to said contacting step is in a composition (e.g., a cell culture medium) comprising a stem cell expander, e.g., described herein, e.g., (S)-2-(6-(2-(1H-indol-3-yl)ethylamino)-2-(5-fluoropyridin-3-yl)-9H-purin-9-yl)propan-1-ol, e.g., (S)-2-(6-(2-(1H-indol-3-yl)ethylamino)-2-(5-fluoropyridin-3-yl)-9H-purin-9-yl)propan-1-ol at a concentration of about 0.25 uM to about 1 uM, e.g., (S)-2-(6-(2-(1H-indol-3-yl)ethylamino)-2-(5-fluoropyridin-3-yl)-9H-purin-9-yl)propan-1-ol at a concentration of about 0.75-0.5 micromolar. In embodiments, the cells are cultured ex vivo for a period of no more than about 1 day, e.g., no more than about 18, 17, 16, 15, 14, 13, 12, 11, 10, 9, 8, 7, 6, 5, 4, 3, 2, or 1 hour(s) after the step of contacting the cells with a CRISPR system, e.g., described herein, e.g., in a cell culture medium which comprises a stem cell expander, e.g., described herein, e.g., (S)-2-(6-(2-(1H-indol-3-yl)ethylamino)-2-(5-fluoropyridin-3-yl)-9H-purin-9-yl)propan-1-ol, e.g., (S)-2-(6-(2-(1H-indol-3-yl)ethylamino)-2-(5-fluoropyridin-3-yl)-9H-purin-9-yl)propan-1-ol at a concentration of about 0.25 uM to about 1 uM, e.g., (S)-2-(6-(2-(1H-indol-3-yl)ethylamino)-2-(5-fluoropyridin-3-yl)-9H-purin-9-yl)propan-1-ol at a concentration of about 0.75-0.5 micromolar. In other embodiments, the cells are cultured ex vivo for a period of about 1 hour to about 14 days, e.g., a period of about 12 hours to about 10 days, e.g., a period of about 1 day to about 10 days, e.g., a period of about 1 day to about 5 days, e.g., a period of about 1 day to about 4 days, e.g., a period of about 2 days to about 4 days, e.g., a period of about 2 days, about 3 days or about 4 days, after the step of contacting the cells with a CRISPR system, e.g., described herein, in a cell culture medium, e.g., which comprises a stem cell expander, e.g., described herein, e.g., (S)-2-(6-(2-(1H-indol-3-yl)ethylamino)-2-(5-fluoropyridin-3-yl)-9H-purin-9-yl)propan-1-ol, e.g., (S)-2-(6-(2-(1H-indol-3-yl)ethylamino)-2-(5-fluoropyridin-3-yl)-9H-purin-9-yl)propan-1-ol at a concentration of about 0.25 uM to about 1 uM, e.g., (S)-2-(6-(2-(1H-indol-3-yl)ethylamino)-2-(5-fluoropyridin-3-yl)-9H-purin-9-yl)propan-1-ol at a concentration of about 0.75-0.5 micromolar.

In embodiments, the cell culture medium is a chemically defined medium. In embodiments, the cell culture medium may additionally contain, for example, StemSpan SFEM (StemCell Technologies; Cat no. 09650). In embodiments, the cell culture medium may alternatively or additionally contain, for example, HSC Brew, GMP (Miltenyi). In embodiments, the cell culture media is serum free. In embodiments, the media may be supplemented with thrombopoietin (TPO), human Flt3 ligand (Flt-3L), human stem cell factor (SCF), human interleukin-6, L-glutamine, and/or penicillin/streptomycin. In embodiments, the media is supplemented with thrombopoietin (TPO), human Flt3 ligand (Flt-3L), human stem cell factor (SCF), human interleukin-6, and L-glutamine. In other embodiments, the media is supplemented with thrombopoietin (TPO), human Flt3 ligand (Flt-3L), human stem cell factor (SCF), and human interleukin-6. In other embodiments the media is supplemented with thrombopoietin (TPO), human Flt3 ligand (Flt-3L), and human stem cell factor (SCF), but not human interleukin-6. In other embodiments, the media is supplemented with human Flt3 ligand (Flt-3L), human stem cell factor (SCF), but not human thrombopoietin (TPO) or human interleukin-6. When present in the medium, the thrombopoietin (TPO), human Flt3 ligand (Flt-3L), human stem cell factor (SCF), human interleukin-6, and/or L-glutamine are each present in a concentration ranging from about 1 ng/mL to about 1000 ng/mL, e.g., a concentration ranging from about 10 ng/mL to about 500 ng/mL, e.g., a concentration ranging from about 10 ng/mL to about 100 ng/mL, e.g., a concentration ranging from about 25 ng/mL to about 75 ng/mL, e.g., a concentration of about 50 ng/mL. In embodiments, each of the supplemented components is at the same concentration. In other embodiments, each of the supplemented components is at a different concentration. In an embodiment, the medium comprises StemSpan SFEM (StemCell Technologies; Cat no. 09650), 50 ng/mL of thrombopoietin (Tpo), 50 ng/mL of human Flt3 ligand (Flt-3L), 50 ng/mL of human stem cell factor (SCF), and 50 ng/mL of human interleukin-6 (IL-6). In an embodiment, the medium comprises StemSpan SFEM (StemCell Technologies; Cat no. 09650), 50 ng/mL of thrombopoietin (Tpo), 50 ng/mL of human Flt3 ligand (Flt-3L), and 50 ng/mL of human stem cell factor (SCF), and does not comprise IL-6. In embodiments, the media further comprises a stem cell expander, e.g., (S)-2-(6-(2-(1H-indol-3-yl)ethylamino)-2-(5-fluoropyridin-3-yl)-9H-purin-9-yl)propan-1-ol, e.g., (S)-2-(6-(2-(1H-indol-3-yl)ethylamino)-2-(5-fluoropyridin-3-yl)-9H-purin-9-yl)propan-1-ol at a concentration of 0.75 μM. In embodiments, the media further comprises a stem cell expander, e.g., (S)-2-(6-(2-(1H-indol-3-yl)ethylamino)-2-(5-fluoropyridin-3-yl)-9H-purin-9-yl)propan-1-ol, e.g., (S)-2-(6-(2-(1H-indol-3-yl)ethylamino)-2-(5-fluoropyridin-3-yl)-9H-purin-9-yl)propan-1-ol at a concentration of 0.5 μM. In embodiments, the media further comprises 1% L-glutamine and 2% penicillin/streptomycin. In embodiments, the cell culture medium is serum free.

### XII. Combination Therapy

The present disclosure contemplates the use of the gRNA molecules described herein, or cells (e.g., hematopoietic stem cells, e.g., CD34+ cells) modified with the gRNA molecules described herein, in combination with one or more other therapeutic modalities and/or agents. Thus, in addition to the use of the gRNA molecules or cells modified with the gRNA molecules described herein, one may also administer to the subject one or more “standard” therapies for treating hemoglobinopathies.

The one or more additional therapies for treating hemoglobinopathies may include, for example, additional stem cell transplantation, e.g., hematopoietic stem cell transplantation. The stem cell transplantation may be allogeneic or autologous.

The one or more additional therapies for treating hemoglobinopathies may include, for example, blood transfusion and/or iron chelation (e.g., removal) therapy. Known iron chelation agents include, for example, deferoxamine and deferasirox.

The one or more additional therapies for treating hemoglobinopathies may include, for example, folic acid supplements, or hydroxyurea (e.g., 5-hydroxyurea). The one or more additional therapies for treating hemoglobinopathies may be hydroxyurea. In embodiments, the hydroxyurea may be administered at a dose of, for example, 10-35 mg/kg per day, e.g., 10-20 mg/kg per day. In embodiments, the hydroxyurea is administered at a dose of 10 mg/kg per day. In embodiments, the hydroxyurea is administered at a dose of 10 mg/kg per day. In embodiments, the hydroxyurea is administered at a dose of 20 mg/kg per day. In embodiments, the hydroxyurea is administered before and/or after the cell (or population of cells), e.g., CD34+ cell (or population of cells) of the invention, e.g., as described herein.

The one or more additional therapeutic agents may include, for example, an anti-p-selectin antibody, e.g., SelG1 (Selexys). P-selectin antibodies are described in, for example, PCT publication WO1993/021956, PCT publication WO1995/034324, PCT publication WO2005/100402, PCT publication WO2008/069999, US patent application publication US2011/0293617, U.S. Pat. Nos. 5,800,815, 6,667,036, 8,945,565, 8,377,440 and 9,068,001, the contents of each of which are incorporated herein in their entirety.

The one or more additional agents may include, for example, a small molecule which upregulates fetal hemoglobin. Examples of such molecules include TN1 (e.g., as described in Nam, T. et al., ChemMedChem 2011, 6, 777-780, DOI: 10.1002/cmdc.201000505, herein incorporated by reference).

The one or more additional therapies may also include irradiation or other bone marrow ablation therapies known in the art. An example of such a therapy is busulfan. Such additional therapy may be performed prior to introduction of the cells of the invention into the subject. In an embodiment the methods of treatment described herein (e.g., the methods of treatment that include administration of cells (e.g., HSPCs) modified by the methods described herein (e.g., modified with a CRISPR system described herein, e.g., to increase HbF production)), the method does not include the step of bone marrow ablation. In embodiments, the methods include a partial bone marrow ablation step.

The therapies described herein (e.g., comprising administering a population of HSPCs, e.g., HSPCs modified using a CRISPR system described herein) may also be combined with an additional therapeutic agent. In an embodiment, the additional therapeutic agent is an HDAC inhibitor, e.g., panobinostat. In an embodiment, the additional therapeutic is a compound described in PCT Publication No. WO2014/150256, e.g., a compound described in Table 1 of WO2014/150256, e.g., GBT440. Other examples of HDAC inhibitors include, for example, suberoylanilide hydroxamic acid (SAHA). The one or more additional agents may include, for example, a DNA methylation inhibitor. Such agents have been shown to increase the HbF induction in cells having reduced BCL 11a activity (e.g., Jian Xu et al, Science 334, 993 (2011); DOI: 0.1126/science.1211053, herein incorporated by reference). Other HDAC inhibitors include any HDAC inhibitor known in the art, for example, trichostatin A, HC toxin, DACI-2, FK228, DACI-14, depudicin, DACI-16, tubacin, NK57, MAZ1536, NK125, Scriptaid, Pyroxamide, MS-275, ITF-2357, MCG-D0103, CRA-024781, CI-994, and LBH589 (see, e.g., Bradner J E, et al., PNAS, 2010 (vol. 107:28), 12617-12622, herein incorporated by reference in its entirety).

The gRNA molecules described herein, or cells (e.g., hematopoietic stem cells, e.g., CD34+ cells) modified with the gRNA molecules described herein, and the co-therapeutic agent or co-therapy can be administered in the same formulation or separately. In the case of separate administration, the gRNA molecules described herein, or cells modified with the gRNA molecules described herein, can be administered before, after or concurrently with the co-therapeutic or co-therapy. One agent may precede or follow administration of the other agent by intervals ranging from minutes to weeks. In embodiments where two or more different kinds of therapeutic agents are applied separately to a subject, one would generally ensure that a significant period of time did not expire between the time of each delivery, such that these different kinds of agents would still be able to exert an advantageously combined effect on the target tissues or cells.

### XIII. Modified Nucleosides, Nucleotides, and Nucleic Acids

Modified nucleosides and modified nucleotides can be present in nucleic acids, e.g., particularly gRNA, but also other forms of RNA, e.g., mRNA, RNAi, or siRNA. As described herein “nucleoside” is defined as a compound containing a five-carbon sugar molecule (a pentose or ribose) or derivative thereof, and an organic base, purine or pyrimidine, or a derivative thereof. As described herein, “nucleotide” is defined as a nucleoside further comprising a phosphate group.

Modified nucleosides and nucleotides can include one or more of:
(i) alteration, e.g., replacement, of one or both of the non-linking phosphate oxygens and/or of one or more of the linking phosphate oxygens in the phosphodiester backbone linkage;
(ii) alteration, e.g., replacement, of a constituent of the ribose sugar, e.g., of the 2′ hydroxyl on the ribose sugar;
(iii) wholesale replacement of the phosphate moiety with “dephospho” linkers;
(iv) modification or replacement of a naturally occurring nucleobase, including with a non-canonical nucleobase;
(v) replacement or modification of the ribose-phosphate backbone;
(vi) modification of the 3′ end or 5′ end of the oligonucleotide, e.g., removal, modification or replacement of a terminal phosphate group or conjugation of a moiety, cap or linker; and
(vii) modification or replacement of the sugar.

The modifications listed above can be combined to provide modified nucleosides and nucleotides that can have two, three, four, or more modifications. For example, a modified nucleoside or nucleotide can have a modified sugar and a modified nucleobase. In an embodiment, every base of a gRNA is modified, e.g., all bases have a modified phosphate group, e.g., all are phosphorothioate groups. In an embodiment, all, or substantially all, of the phosphate groups of a unimolecular or modular gRNA molecule are replaced with phosphorothioate groups. In embodiments, one or more of the five 3′-terminal bases and/or one or more of the five 5′-terminal bases of the gRNA are modified with a phosphorothioate group.

In an embodiment, modified nucleotides, e.g., nucleotides having modifications as described herein, can be incorporated into a nucleic acid, e.g., a “modified nucleic acid.” In some embodiments, the modified nucleic acids comprise one, two, three or more modified nucleotides. In some embodiments, at least 5% (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the positions in a modified nucleic acid are a modified nucleotides.

Unmodified nucleic acids can be prone to degradation by, e.g., cellular nucleases. For example, nucleases can hydrolyze nucleic acid phosphodiester bonds. Accordingly, in one aspect the modified nucleic acids described herein can contain one or more modified nucleosides or nucleotides, e.g., to introduce stability toward nucleases.

In some embodiments, the modified nucleosides, modified nucleotides, and modified nucleic acids described herein can exhibit a reduced innate immune response when introduced into a population of cells, both in vivo and ex vivo. The term “innate immune response” includes a cellular response to exogenous nucleic acids, including single stranded nucleic acids, generally of viral or bacterial origin, which involves the induction of cytokine expression and release, particularly the interferons, and cell death. In some embodiments, the modified nucleosides, modified nucleotides, and modified nucleic acids described herein can disrupt binding of a major groove interacting partner with the nucleic acid. In some embodiments, the modified nucleosides, modified nucleotides, and modified nucleic acids described herein can exhibit a reduced innate immune response when introduced into a population of cells, both in vivo and ex vivo, and also disrupt binding of a major groove interacting partner with the nucleic acid.

### Definitions of Chemical Groups

As used herein, “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 20, from 2 to about 20, from 1 to about 12, from 1 to about 8, from 1 to about 6, from 1 to about 4, or from 1 to about 3 carbon atoms.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “alkenyl” refers to an aliphatic group containing at least one double bond. As used herein, “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl.

As used herein, “arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

As used herein, “cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl.

As used herein, “heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl.

As used herein, “heteroaryl” refers to a monovalent radical of a heteroaromatic ring system. Examples of heteroaryl moieties include, but are not limited to, imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

### Phosphate Backbone Modifications

### The Phosphate Group

In some embodiments, the phosphate group of a modified nucleotide can be modified by replacing one or more of the oxygens with a different substituent. Further, the modified nucleotide, e.g., modified nucleotide present in a modified nucleic acid, can include the wholesale replacement of an unmodified phosphate moiety with a modified phosphate as described herein. In some embodiments, the modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of the Phosphate Group

The phosphate group can be replaced by non-phosphorus containing connectors. In some embodiments, the charge phosphate group can be replaced by a neutral moiety.

Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Replacement of the Ribophosphate Backbone

Scaffolds that can mimic nucleic acids can also be constructed wherein the phosphate linker and ribose sugar are replaced by nuclease resistant nucleoside or nucleotide surrogates. In some embodiments, the nucleobases can be tethered by a surrogate backbone. Examples can include, without limitation, the morpholino, cyclobutyl, pyrrolidine and peptide nucleic acid (PNA) nucleoside surrogates.

### Sugar Modifications

The modified nucleosides and modified nucleotides can include one or more modifications to the sugar group. For example, the 2′ hydroxyl group (OH) can be modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom.

Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include “locked” nucleic acids (LNA) in which the 2′ hydroxyl can be connected, e.g., by a Ci-₆ alkylene or Cj-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)˜-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH₃, e.g., a PEG derivative).

“Deoxy” modifications can include hydrogen (i.e. deoxyribose sugars, e.g., at the overhang portions of partially ds RNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the Γ position on the sugar, e.g., alpha-nucleosides. The modified nucleic acids can also include “abasic” sugars, which lack a nucleobase at C−. These abasic sugars can also be further modified at one or more of the constituent sugar atoms. The modified nucleic acids can also include one or more sugars that are in the L form, e.g. L-nucleosides.

Generally, RNA includes the sugar group ribose, which is a 5-membered ring having an oxygen. Exemplary modified nucleosides and modified nucleotides can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replaced with a-L-threofuranosyl-(3′↔2′)).

### Modifications on the Nucleobase

The modified nucleosides and modified nucleotides described herein, which can be incorporated into a modified nucleic acid, can include a modified nucleobase. Examples of nucleobases include, but are not limited to, adenine (A), guanine (G), cytosine (C), and uracil (U). These nucleobases can be modified or wholly replaced to provide modified nucleosides and modified nucleotides that can be incorporated into modified nucleic acids. The nucleobase of the nucleotide can be independently selected from a purine, a pyrimidine, a purine or pyrimidine analog. In some embodiments, the nucleobase can include, for example, naturally-occurring and synthetic derivatives of a base.

### Uracil

In some embodiments, the modified nucleobase is a modified uracil. Exemplary nucleobases and nucleosides having a modified uracil include without limitation pseudouridine (Ψ), pyridin-4-one ribonucleoside, 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-u,ridine (ho⁵U), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmosU), uridine 5-oxyacetic acid methyl ester (mcmo\hat{ }U), 5-carboxymethyl-uridine (cmSU), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s2U), 5-aminomethyl-2-thio-uridine (nm⁵s2U), 5-methylaminomethyl-uridine (mnmU), 5-methylaminomethyl-2-thio-uridine (mnm⁵s2U), 5-methylaminomethyl-2-seleno-uridine (mnmnse²U), 5-carbamoylmethyl-uridine (ncmsU), 5-carboxymethylaminomethyl-uridine (cmnm⁵U), 5-carboxymethylaminomethyl-2-thio-uridine (cmnm s2U), 5-propynyl-uridine, 1-propynyl-pseudouridine, 5-taurinomethyl-uridine (xcmU), 1-taurinomethyl-pseudouridine, 5-taurinomethyl-2-thio-uridine(Trn⁵2U), 1-taurinomethyl-4-thio-pseudouridine, 5-methyl-uridine (m⁵U, i.e., having the nucleobase deoxythymine), 1-methyl-pseudouridine (ιτι′Ψ). 5-methyl-2-thio-uridine (m⁵s2U), 1-methyl-4-thio-pseudouridine (m′s \|/), 4-thio-1-methyl-pseudouridine, 3-methyl-pseudouridine (m′V), 2-thio-1-methyl-pseudouridine, 1-methyl-1-deaza-pseudouridine, 2-thio-1-methyl-1-deaza-pseudouridine, dihydroundine (D), dihydropseudoundine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N 1-methyl-pseudouridine, 3-(3-amino-3-carboxypropyl)uridine (acp³U), 1-methyl-3-(3-amino-3-carboxypropy pseudouridine 5-(isopentenylaminomethyl)uridine (inmU), 5-(isopentenylaminomethy])-2-thio-uridine (inm⁵s2U), a-thio-uridine, 2-O-methyl-uridine (Urn), 5,2-O-dimethyl-uridine (m⁵Um), 2-O-methyl-pseudouridine (105 πι), 2-thio-2′-O-methyl-uridine (s2Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

### Cytosine

In some embodiments, the modified nucleobase is a modified cytosine. Exemplary nucleobases and nucleosides having a modified cytosine include without limitation 5-aza-cytidine, 6-aza-cytidine, pseudoisocytidine, 3-methyl-cytidine (m³C), N4-acetyl-cytidine (act), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s2C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k²C), a-thio-cytidine, 2′-O-methyl-cytidine (Cm), 5,2′-0-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

### Adenine

In some embodiments, the modified nucleobase is a modified adenine. Exemplary nucleobases and nucleosides having a modified adenine include without limitation 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m′A), 2-methyl-adenine (m A), N6-methyl-adenosine (m⁶A), 2-methylthio-N6-methyl-adenosine (ms² m⁶A), N6-isopentenyl-adenosine (i⁶A), 2-methylthio-N6-isopentenyl-adenosine (ms²io⁶A), N6-(cis-hydroxyisopentenyl)adenos′ine (io⁶A), 2-methylthio-N6-(cis-hydroxyisopentenyl)adenosine (ms2io⁶A), N6-glycinylcarbamoyl-adenosine (g⁶A), N6-threonylcarbamoyl-adenosine (t⁶A), N6-methyl-N6-threonylcarbamoyl-adenosine (m⁶t6A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms2hn⁶A), N6-acetyl-adenosine (ac⁶A), 7-methyl-adenine, 2-methylthio-adenine, 2-methoxy-adenine, a-thio-adenosine, 2′-O-methyl-adenosine (Am), N⁶,2′-O-dimethyl-adenosine (m⁵Am), N⁶-Methyl-2′-deoxyadenosine, N6,N6,2′-0-trimethyl-adenosine (m⁶₂Am), 1,2′-O-dimethyl-adenosine (m′Am), 2′-0-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

### Guanine

In some embodiments, the modified nucleobase is a modified guanine. Exemplary nucleobases and nucleosides having a modified guanine include without limitation inosine (I), 1-methyl-inosine (m ′l), wyosine (imG), methylwyosine (mimG), 4-demethyl-wyo″sine (imG-14), isowyosine (imG2), wybutosine (yW), peroxywybutosine (O₂yW), hydroxywybutosine (OHyW), undemriodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQo), 7-aminomethyI-7-deaza-guanosine (preQi), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m′G), N2-methyl-guanosine (m²G), N2,N2-dimethyl-guanosine (m²₂G), N2,7-dimethyl-guanosine (m²,7G), N2, N2,7-dimethyl-guanosine (m²,2,7G), 8-oxo-guanosine, 7-methyl-8-oxo-guanosine, 1-meth thio-guanosine, N2-methyl-6-thio-guanosine, N2,N2-dimethyl-6-thio-guanosine, a-thio-guanosine, 2′-O-methyl-guanosine (Gm), N2-methyl-2′-O-methyl-guanosine (m¾m), N2,N2-dimethyl-2′-O-methyl-guanosine (m²₂Gm), 1-methyl-2′-O-methyl-guanosine (m′Gm), N2,7-dimethyl-2′-O-methyl-guanosine (m²,7Gm), 2′-O-methyl-inosine (Im), 1,2′-O-dimethyl-inosine (m′lm), O⁶-phenyl-2′-deoxyinosine, 2′-0-ribosylguanosine (phosphate) (Gr(p)), 1-thio-guanosine, O⁶-methy]-guanosine, O⁶-Methyl-2′-deoxyguanosine, 2′-F-ara-guanosine, and 2′-F-guanosine.

Modified gRNAs

In some embodiments, the modified nucleic acids can be modified gRNAs. In some embodiments, gRNAs can be modified at the 3′ end. In this embodiment, the gRNAs can be modified at the 3′ terminal U ribose. For example, the two terminal hydroxyl groups of the U ribose can be oxidized to aldehyde groups and a concomitant opening of the ribose ring to afford a modified nucleoside, wherein U can be an unmodified or modified uridine.

In another embodiment, the 3′ terminal U can be modified with a 2′ 3′ cyclic phosphate, wherein U can be an unmodified or modified uridine. In some embodiments, the gRNA molecules may contain 3′ nucleotides which can be stabilized against degradation, e.g., by incorporating one or more of the modified nucleotides described herein. In this embodiment, e.g., uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the gRNA. In some embodiments, O- and N-alkylated nucleotides, e.g., N6-methyl adenosine, can be incorporated into the gRNA. In some embodiments, sugar-modified ribonucleotides can be incorporated, e.g., wherein the 2′ OH— group is replaced by a group selected from H, —OR, —R (wherein R can be, e.g., methyl, alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, the nucleotides in the overhang region of the gRNA can each independently be a modified or unmodified nucleotide including, but not limited to 2′-sugar modified, such as, 2-F 2′-0-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof.

In an embodiment, one or more or all of the nucleotides in single stranded overhang of an RNA molecule, e.g., a gRNA molecule, are deoxynucleotides.

### XIV. Pharmaceutical Compositions

Pharmaceutical compositions of the present invention may comprise a gRNA molecule described herein, e.g., a plurality of gRNA molecules as described herein, or a cell (e.g., a population of cells, e.g., a population of hematopoietic stem cells, e.g., of CD34+ cells) comprising one or more cells modified with one or more gRNA molecules described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminium hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, mouse antibodies, pooled human serum, bovine serum albumin, bovine serum, culture media components, unwanted CRISPR system components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

The administration of the subject compositions may be carried out in any convenient manner, including by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In one aspect, the compositions of the present invention are administered to a patient by intradermal or subcutaneous injection. In one aspect, the cell compositions of the present invention are administered by i.v. injection.

The dosage of the above treatments to be administered to a patient will vary with the precise nature of the conditionbeing treated and the recipient of the treatment. The scaling of dosages for human administration can be performed according to art-accepted practices.

### XV. Cells

The invention also relates to cells comprising a gRNA molecule of the invention, or nucleic acid encoding said gRNA molecules.

In an aspect the cells are cells made by a process described herein.

In embodiments, the cells are hematopoietic stem cells (e.g., hematopoietic stem and progenitor cells; HSPCs), for example, CD34+ stem cells. In embodiments, the cells are CD34+/CD90+ stem cells. In embodiments, the cells are CD34+/CD90− stem cells. In embodiments, the cells are human hematopoietic stem cells. In embodiments, the cells are autologous. In embodiments, the cells are allogeneic.

In embodiments, the cells are derived from bone marrow, e.g., autologous bone marrow. In embodiments, the cells are derived from peripheral blood, e.g., mobilized peripheral blood, e.g., autologous mobilized peripheral blood. In embodiments employing mobilized peripheral blood, the cells are isolated from patients who have been administered a mobilization agent. In embodiments, the mobilization agent is G-CSF. In embodiments, the mobilization agent is Plerixafor® (AIMD3100). In embodiments, the mobilization agent comprises a combination of G-CSF and Plerixafor® (AMD3100)). In embodiments, the cells are derived from umbilical cord blood, e.g., allogeneic umbilical cord blood. In embodiments, the cells are derived from a hemoglobinopathy patient, for example a patient with sickle cell disease or a patient with a thalassemia, e.g., beta-thalassemia.

In embodiments, the cells are mammalian. In embodiments, the cells are human. In embodiments, the cells are derived from a hemoglobinopathy patient, for example a patient with sickle cell disease or a patient with a thalassemia, e.g., beta-thalassemia.

In an aspect, the invention provides a cell comprising a modification or alteration, e.g., an indel, at or near (e.g., within 20, 19, 18, 17, 16, 15, 14, 13, 12, 11, 10, 9, 8, 7, 6, 5, 4, 3, 2 or 1 nucleotides of) a nucleic acid sequence having complementarity to a gRNA molecule or gRNA molecules, e.g., as described herein, introduced into said cells, e.g., as part of a CRISPR system as described herein. In embodiments, the cell is a CD34+ cell. In embodiments, the altered or modified cell, e.g., CD34+ cell, maintains the ability to differentiate into cells of multiple lineages, e.g., maintains the ability to differentiate into cells of the erythroid lineage. In embodiments, the altered or modified cell, e.g., CD34+ cell, has undergone or is able to undergo at least 2, at least 3, at least 4, at least 5, at least 6, at least 7, at least 8, at least 9 or at least 10 or more doublings in culture, e.g., in culture comprising a stem cell expander, e.g., (S)-2-(6-(2-(1H-indol-3-yl)ethylamino)-2-(5-fluoropyridin-3-yl)-9H-purin-9-yl)propan-1-ol. In embodiments, the altered or modified cell, e.g., CD34+ cell, has undergone or is able to undergo at least 5, e.g., about 5, doublings in culture, e.g., in culture comprising a stem cell expander molecule, e.g., as described herein, e.g., (S)-2-(6-(2-(1H-indol-3-yl)ethylamino)-2-(5-fluoropyridin-3-yl)-9H-purin-9-yl)propan-1-ol. In embodiments the altered or modified cell, e.g., CD34+ cell, exhibits and/or is able to differentiate into a cell, e.g., into a cell of the erythroid lineage, e.g., into a red blood cell, that exhibits increased fetal hemoglobin level (e.g., expression level and/or protein level), e.g., at least a 20% increase in fetal hemoglobin protein level, relative to a similar unmodified or unaltered cell. In embodiments the altered or modified cell, e.g., CD34+ cell, exhibits and/or is able to differentiate into a cell, e.g., into a cell of the erythroid lineage, e.g., into a red blood cell, that exhibits increased fetal hemoglobin level (e.g., expression level and/or protein level), relative to a similar unmodified or unaltered cell, e.g., produces at least 6 picograms, e.g., at least 7 picograms, at least 8 picograms, at least 9 picograms, or at least 10 picograms of fetal hemoglobin. In embodiments the altered or modified cell, e.g., CD34+ cell, exhibits and/or is able to differentiate into a cell, e.g., into a cell of the erythroid lineage, e.g., into a red blood cell, that exhibits increased fetal hemoglobin level (e.g., expression level and/or protein level), relative to a similar unmodified or unaltered cell, e.g., produces about 6 to about 12, about 6 to about 7, about 7 to about 8, about 8 to about 9, about 9 to about 10, about 10 to about 11 or about 11 to about 12 picograms of fetal hemoglobin.

In an aspect, the invention provides a population of cells comprising cells having a modification or alteration, e.g., an indel, at or near (e.g., within 20, 19, 18, 17, 16, 15, 14, 13, 12, 11, 10, 9, 8, 7, 6, 5, 4, 3, 2 or 1 nucleotides of) a nucleic acid sequence having complementarity to a gRNA molecule or gRNA molecules, e.g., as described herein, introduced into said cells, e.g., as part of a CRISPR system as described herein. In embodiments, at least 50%, e.g., at least 60%, at least 70%, at least 80% or at least 90% of the cells of the population have the modification or alteration (e.g., have at least one modification or alteration), e.g., as measured by NGS, e.g., as described herein, e.g., at day two following introduction of the gRNA and/or CRISPR system of the invention. In embodiments, at least 90%, e.g., at least 91%, at least 92%, at least 93%, at least 94%, at least 95%, at least 96%, at least 97%, at least 98% or at least 99% of the cells of the population have the modification or alteration (e.g., have at least one modification or alteration), e.g., as measured by NGS, e.g., as described herein, e.g., at day two following introduction of the gRNA and/or CRISPR system of the invention. In embodiments, the population of cells comprise CD34+ cells, e.g., comprise at least about 50%, at least about 60%, at least about 70%, at least about 80%, at least about 90%, at least about 95% or at least about 98% CD34+ cells. In embodiments, the population of cells comprising the altered or modified cells, e.g., CD34+ cells, maintain the ability to produce, e.g., differentiate into, cells of multiple lineages, e.g., maintains the ability to produce, e.g., differentiate into, cells of the erythroid lineage. In embodiments, the population of cells, e.g., population of CD34+ cells, has undergone or is able to undergo at least 2, at least 3, at least 4, at least 5, at least 6, at least 7, at least 8, at least 9 or at least 10 or more population doublings in culture, e.g., in culture comprising a stem cell expander, e.g., (S)-2-(6-(2-(1H-indol-3-yl)ethylamino)-2-(5-fluoropyridin-3-yl)-9H-purin-9-yl)propan-1-ol. In embodiments, the population of altered or modified cells, e.g., population of CD34+ cells, has undergone or is capable of undergoing at least 5, e.g., about 5, population doublings in culture, e.g., in culture comprising a stem cell expander molecule, e.g., as described herein, e.g., (S)-2-(6-(2-(1H-indol-3-yl)ethylamino)-2-(5-fluoropyridin-3-yl)-9H-purin-9-yl)propan-1-ol. In embodiments the population of cells comprising altered or modified cells, e.g., CD34+ cells, exhibits and/or is able to differentiate into a population of cells, e.g., into a population of cells of the erythroid lineage, e.g., into a population of red blood cells, that exhibits increased fetal hemoglobin level (e.g., expression level and/or protein level), e.g., at least a 20% increase in fetal hemoglobin protein level, relative to a similar unmodified or unaltered cells. In embodiments the population of cells comprising altered or modified cells, e.g., CD34+ cells, exhibits and/or is able to differentiate into a population of cells, e.g., into a population of cells of the erythroid lineage, e.g., into a population of red blood cells, that exhibits increased fetal hemoglobin level (e.g., expression level and/or protein level), relative to a similar unmodified or unaltered cells, e.g., comprises cells that produce at least 6 picograms, e.g., at least 7 picograms, at least 8 picograms, at least 9 picograms, or at least 10 picograms of fetal hemoglobin per cell. In embodiments the population of altered or modified cells, e.g., CD34+ cells, exhibits and/or is able to differentiate into a population of cells, e.g., into a population of cells of the erythroid lineage, e.g., into a population of red blood cells, that exhibits increased fetal hemoglobin level (e.g., expression level and/or protein level), relative to a similar unmodified or unaltered cell, e.g., comprises cells that produce about 6 to about 12, about 6 to about 7, about 7 to about 8, about 8 to about 9, about 9 to about 10, about 10 to about 11 or about 11 to about 12 picograms of fetal hemoglobin per cell.

In embodiments, the population of cells, e.g., as described herein, comprises at least about 1e3 cells. In embodiments, the population of cells, e.g., as described herein, comprises at least about 1e4 cells. In embodiments, the population of cells, e.g., as described herein, comprises at least about 1e5 cells. In embodiments, the population of cells, e.g., as described herein, comprises at least about 1e6 cells. In embodiments, the population of cells, e.g., as described herein, comprises at least about 1e7 cells. In embodiments, the population of cells, e.g., as described herein, comprises at least about 1e8 cells. In embodiments, the population of cells, e.g., as described herein, comprises at least about 1e9 cells. In embodiments, the population of cells, e.g., as described herein, comprises at least about 1e10 cells. In embodiments, the population of cells, e.g., as described herein, comprises at least about 1e11 cells. In embodiments, the population of cells, e.g., as described herein, comprises at least about 1e12 cells. In embodiments, the population of cells, e.g., as described herein, comprises at least about 1e13 cells per kilogram body weight of the patient to which they are to be administered. In embodiments, the population of cells, e.g., as described herein, comprises at least about 1e6 cells per kilogram body weight of the patient to which they are to be administered. In embodiments, the population of cells, e.g., as described herein, comprises at least about 2e6 cells per kilogram body weight of the patient to which they are to be administered. In embodiments, the population of cells, e.g., as described herein, comprises at least about 3e6 cells per kilogram body weight of the patient to which they are to be administered. In embodiments, the population of cells, e.g., as described herein, comprises at least about 4e6 cells per kilogram body weight of the patient to which they are to be administered. In embodiments, the population of cells, e.g., as described herein, comprises at least about 5e6 cells per kilogram body weight of the patient to which they are to be administered. In embodiments, the population of cells, e.g., as described herein, comprises at least about 6e6 cells per kilogram body weight of the patient to which they are to be administered. In embodiments, the population of cells, e.g., as described herein, comprises at least about 7e6 cells per kilogram body weight of the patient to which they are to be administered. In embodiments, the population of cells, e.g., as described herein, comprises at least about 8e6 cells per kilogram body weight of the patient to which they are to be administered. In embodiments, the population of cells, e.g., as described herein, comprises at least about 9e6 cells per kilogram body weight of the patient to which they are to be administered. In embodiments, the population of cells, e.g., as described herein, comprises at least about 1e7 cells per kilogram body weight of the patient to which they are to be administered. In embodiments, the population of cells, e.g., as described herein, comprises at least about 2e7 cells per kilogram body weight of the patient to which they are to be administered. In embodiments, the population of cells, e.g., as described herein, comprises at least about 3e7 cells per kilogram body weight of the patient to which they are to be administered. In embodiments, the population of cells, e.g., as described herein, comprises at least about 4e7 cells per kilogram body weight of the patient to which they are to be administered. In embodiments, the population of cells, e.g., as described herein, comprises at least about 5e7 cells per kilogram body weight of the patient to which they are to be administered. In embodiments, the population of cells, e.g., as described herein, comprises at least about 6e7 cells per kilogram body weight of the patient to which they are to be administered. In embodiments, the population of cells, e.g., as described herein, comprises at least about 7e7 cells per kilogram body weight of the patient to which they are to be administered. In embodiments, the population of cells, e.g., as described herein, comprises at least about 8e7 cells per kilogram body weight of the patient to which they are to be administered. In embodiments, the population of cells, e.g., as described herein, comprises at least about 9e7 cells per kilogram body weight of the patient to which they are to be administered. In embodiments, the population of cells, e.g., as described herein, comprises at least about 1e8 cells per kilogram body weight of the patient to which they are to be administered. In any of the aforementioned embodiments, the population of cells may comprise at least about 50% (for example, at least about 60%, at least about 70%, at least about 80%, at least about 90%, at least about 95% or at least about 99%) HSPCs, e.g., CD34+ cells. In any of the aforementioned embodiments, the population of cells may comprise about 60% HSPCs, e.g., CD34+ cells. In an embodiment, the population of cells, e.g., as described herein, comprises about 3e7 cells and comprises about 2e7 HSPCs, e.g., CD34+ cells. As used throughout this application, the scientific notation [number]e[number] is given its ordinary meaning. Thus, for example, 2e6 is equivalent to 2×10⁶ or 2,000,000. In embodiments, the population of cells, e.g., as described herein, comprises at least about 1e6 HSPCs, e.g., CD34+ cells, per kilogram body weight of the patient to which they are to be administered. In embodiments, the population of cells, e.g., as described herein, comprises at least about 1.5e6 HSPCs, e.g., CD34+ cells, per kilogram body weight of the patient to which they are to be administered. In embodiments, the population of cells, e.g., as described herein, comprises at least about 2e6 HSPCs, e.g., CD34+ cells, per kilogram body weight of the patient to which they are to be administered. In embodiments, the population of cells, e.g., as described herein, comprises at least about 3e6 HSPCs, e.g., CD34+ cells, per kilogram body weight of the patient to which they are to be administered. In embodiments, the population of cells, e.g., as described herein, comprises at least about 4e6 HSPCs, e.g., CD34+ cells, per kilogram body weight of the patient to which they are to be administered. In embodiments, the population of cells, e.g., as described herein, comprises at least about 5e6 HSPCs, e.g., CD34+ cells, per kilogram body weight of the patient to which they are to be administered. In embodiments, the population of cells, e.g., as described herein, comprises at least about 6e6 HSPCs, e.g., CD34+ cells, per kilogram body weight of the patient to which they are to be administered. In embodiments, the population of cells, e.g., as described herein, comprises at least about 7e6 HSPCs, e.g., CD34+ cells, per kilogram body weight of the patient to which they are to be administered. In embodiments, the population of cells, e.g., as described herein, comprises at least about 8e6 HSPCs, e.g., CD34+ cells, per kilogram body weight of the patient to which they are to be administered. In embodiments, the population of cells, e.g., as described herein, comprises at least about 9e6 HSPCs, e.g., CD34+ cells, per kilogram body weight of the patient to which they are to be administered. In embodiments, the population of cells, e.g., as described herein, comprises at least about 1e7 HSPCs, e.g., CD34+ cells, per kilogram body weight of the patient to which they are to be administered. In embodiments, the population of cells, e.g., as described herein, comprises at least about 2e7 HSPCs, e.g., CD34+ cells, per kilogram body weight of the patient to which they are to be administered. In embodiments, the population of cells, e.g., as described herein, comprises at least about 3e7 HSPCs, e.g., CD34+ cells, per kilogram body weight of the patient to which they are to be administered. In embodiments, the population of cells, e.g., as described herein, comprises at least about 4e7 HSPCs, e.g., CD34+ cells, per kilogram body weight of the patient to which they are to be administered. In embodiments, the population of cells, e.g., as described herein, comprises at least about 5e7 HSPCs, e.g., CD34+ cells, per kilogram body weight of the patient to which they are to be administered. In embodiments, the population of cells, e.g., as described herein, comprises at least about 6e7 HSPCs, e.g., CD34+ cells, per kilogram body weight of the patient to which they are to be administered. In embodiments, the population of cells, e.g., as described herein, comprises at least about 7e7 HSPCs, e.g., CD34+ cells, per kilogram body weight of the patient to which they are to be administered. In embodiments, the population of cells, e.g., as described herein, comprises at least about 8e7 HSPCs, e.g., CD34+ cells, per kilogram body weight of the patient to which they are to be administered. In embodiments, the population of cells, e.g., as described herein, comprises at least about 9e7 HSPCs, e.g., CD34+ cells, per kilogram body weight of the patient to which they are to be administered. In embodiments, the population of cells, e.g., as described herein, comprises at least about 1e8 HSPCs, e.g., CD34+ cells, per kilogram body weight of the patient to which they are to be administered. In embodiments, the population of cells, e.g., as described herein, comprises at least about 2e8 HSPCs, e.g., CD34+ cells, per kilogram body weight of the patient to which they are to be administered. In embodiments, the population of cells, e.g., as described herein, comprises at least about 3e8 HSPCs, e.g., CD34+ cells, per kilogram body weight of the patient to which they are to be administered. In embodiments, the population of cells, e.g., as described herein, comprises at least about 4e8 HSPCs, e.g., CD34+ cells, per kilogram body weight of the patient to which they are to be administered. In embodiments, the population of cells, e.g., as described herein, comprises at least about 5e8 HSPCs, e.g., CD34+ cells, per kilogram body weight of the patient to which they are to be administered.

In embodiments, the population of cells, e.g., as described herein, comprises about 1e6 HSPCs, e.g., CD34+ cells, per kilogram body weight of the patient to which they are to be administered. In embodiments, the population of cells, e.g., as described herein, comprises about 1.5e6 HSPCs, e.g., CD34+ cells, per kilogram body weight of the patient to which they are to be administered. In embodiments, the population of cells, e.g., as described herein, comprises about 2e6 HSPCs, e.g., CD34+ cells, per kilogram body weight of the patient to which they are to be administered. In embodiments, the population of cells, e.g., as described herein, comprises about 3e6 HSPCs, e.g., CD34+ cells, per kilogram body weight of the patient to which they are to be administered. In embodiments, the population of cells, e.g., as described herein, comprises about 4e6 HSPCs, e.g., CD34+ cells, per kilogram body weight of the patient to which they are to be administered. In embodiments, the population of cells, e.g., as described herein, comprises about 5e6 HSPCs, e.g., CD34+ cells, per kilogram body weight of the patient to which they are to be administered. In embodiments, the population of cells, e.g., as described herein, comprises about 6e6 HSPCs, e.g., CD34+ cells, per kilogram body weight of the patient to which they are to be administered. In embodiments, the population of cells, e.g., as described herein, comprises about 7e6 HSPCs, e.g., CD34+ cells, per kilogram body weight of the patient to which they are to be administered. In embodiments, the population of cells, e.g., as described herein, comprises about 8e6 HSPCs, e.g., CD34+ cells, per kilogram body weight of the patient to which they are to be administered. In embodiments, the population of cells, e.g., as described herein, comprises about 9e6 HSPCs, e.g., CD34+ cells, per kilogram body weight of the patient to which they are to be administered. In embodiments, the population of cells, e.g., as described herein, comprises about 1e7 HSPCs, e.g., CD34+ cells, per kilogram body weight of the patient to which they are to be administered. In embodiments, the population of cells, e.g., as described herein, comprises about 2e7 HSPCs, e.g., CD34+ cells, per kilogram body weight of the patient to which they are to be administered. In embodiments, the population of cells, e.g., as described herein, comprises about 3e7 HSPCs, e.g., CD34+ cells, per kilogram body weight of the patient to which they are to be administered. In embodiments, the population of cells, e.g., as described herein, comprises about 4e7 HSPCs, e.g., CD34+ cells, per kilogram body weight of the patient to which they are to be administered. In embodiments, the population of cells, e.g., as described herein, comprises about 5e7 HSPCs, e.g., CD34+ cells, per kilogram body weight of the patient to which they are to be administered. In embodiments, the population of cells, e.g., as described herein, comprises about 6e7 HSPCs, e.g., CD34+ cells, per kilogram body weight of the patient to which they are to be administered. In embodiments, the population of cells, e.g., as described herein, comprises about 7e7 HSPCs, e.g., CD34+ cells, per kilogram body weight of the patient to which they are to be administered. In embodiments, the population of cells, e.g., as described herein, comprises about 8e7 HSPCs, e.g., CD34+ cells, per kilogram body weight of the patient to which they are to be administered. In embodiments, the population of cells, e.g., as described herein, comprises about 9e7 HSPCs, e.g., CD34+ cells, per kilogram body weight of the patient to which they are to be administered. In embodiments, the population of cells, e.g., as described herein, comprises about 1e8 HSPCs, e.g., CD34+ cells, per kilogram body weight of the patient to which they are to be administered. In embodiments, the population of cells, e.g., as described herein, comprises about 2e8 HSPCs, e.g., CD34+ cells, per kilogram body weight of the patient to which they are to be administered. In embodiments, the population of cells, e.g., as described herein, comprises about 3e8 HSPCs, e.g., CD34+ cells, per kilogram body weight of the patient to which they are to be administered. In embodiments, the population of cells, e.g., as described herein, comprises about 4e8 HSPCs, e.g., CD34+ cells, per kilogram body weight of the patient to which they are to be administered. In embodiments, the population of cells, e.g., as described herein, comprises about 5e8 HSPCs, e.g., CD34+ cells, per kilogram body weight of the patient to which they are to be administered.

In embodiments, the population of cells, e.g., as described herein, comprises from about 2e6 to about 10e6 HSPCs, e.g., CD34+ cells, per kilogram body weight of the patient to which they are to be administered. In embodiments, the population of cells, e.g., as described herein, comprises from 2e6 to 10e6 HSPCs, e.g., CD34+ cells, per kilogram body weight of the patient to which they are to be administered.

The cells of the invention may comprise a gRNA molecule of the present invention, or nucleic acid encoding said gRNA molecule, and a Cas9 molecule of the present invention, or nucleic acid encoding said Cas9 molecule. In an embodiment, the cells of the invention may comprise a ribonuclear protein (RNP) complex which comprises a gRNA molecule of the invention and a Cas9 molecule of the invention.

The cells of the invention are preferably modified to comprise a gRNA molecule of the invention ex vivo, for example by a method described herein, e.g., by electroporation or by TRIAMF (as described in patent application PCT/US2017/54110, incorporated herein by reference in its entirety).

The cells of the invention include cells in which expression of one or more genes has been altered, for example, reduced or inhibited, by introduction of a CRISPR system comprising a gRNA of the invention. For example, the cells of the present invention may have a reduced level of beta globin (e.g., hemoglobin beta comprising a sickling mutation) expression relative to unmodified cells. As another example, the cells of the present invention may have an increased level of fetal hemoglobin expression relative to unmodified cells. Alternatively, or in addition, a cell of the invention may give rise, e.g., differentiate into, another type of cell, e.g., an erythrocyte, that has an increased level of fetal hemoglobin expression relative to cells differentiated from unmodified cells. In embodiments, the increase in level of fetal hemoglobin is at least about 20%, at least about 30%, at least about 40% or at least about 50%. Alternatively, or in addition, a cell of the invention may give rise, e.g., differentiate into, another type of cell, e.g., an erythrocyte, that has a reduced level of beta globin (e.g., hemoglobin beta comprising a sickling mutation, also referred to herein as sickle beta globin) expression relative to cells differentiated from unmodified cells. In embodiments, the decrease in level of sickle beta-globin is at least about 20%, at least about 30%, at least about 40% or at least about 50%.

The cells of the invention include cells in which expression of one or more genes has been altered, for example, reduced or inhibited, by introduction of a CRISPR system comprising a gRNA of the invention. For example, the cells of the present invention may have a reduced level of hemoglobin beta, for example a mutated or wild-type hemoglobin beta, expression relative to unmodified cells. In another aspect, the invention provides cells which are derived from, e.g., differentiated from, cells in which a CRISPR system comprising a gRNA of the invention has been introduced. In such aspects, the cells in which the CRISPR system comprising the gRNA of the invention has been introduced may not exhibit the reduced level of hemoglobin beta, for example a mutated or wild-type hemoglobin beta, but the cells derived from, e.g., differentiated from, said cells exhibit the reduced level of hemoglobin beta, for example a mutated or wild-type hemoglobin beta. In embodiments, the derivation, e.g., differentiation, is accomplished in vivo (e.g., in a patient, e.g., in a hemoglobinopathy patient, e.g., in a patient with sickle cell disease or a thalassemia, e.g., beta thalassemia). In embodiments the cells in which the CRISPR system comprising the gRNA of the invention has been introduced are CD34+ cells and the cells derived, e.g., differentiated, therefrom are of the erythroid lineage, e.g., red blood cells.

The cells of the invention include cells in which expression of one or more genes has been altered, for example, increased or promoted, by introduction of a CRISPR system comprising a gRNA of the invention. For example, the cells of the present invention may have an increased level of fetal hemoglobin expression relative to unmodified cells. In another aspect, the invention provides cells which are derived from, e.g., differentiated from, cells in which a CRISPR system comprising a gRNA of the invention has been introduced. In such aspects, the cells in which the CRISPR system comprising the gRNA of the invention has been introduced may not exhibit the increased level of fetal hemoglobin but the cells derived from, e.g., differentiated from, said cells exhibit the increased level of fetal hemoglobin. In embodiments, the derivation, e.g., differentiation, is accomplished in vivo (e.g., in a patient, e.g., in a hemoglobinopathy patient, e.g., in a patient with sickle cell disease or a thalassemia, e.g., beta thalassemia). In embodiments the cells in which the CRISPR system comprising the gRNA of the invention has been introduced are CD34+ cells and the cells derived, e.g., differentiated, therefrom are of the erythroid lineage, e.g., red blood cells.

In another aspect, the invention relates to cells which include an indel at (e.g., within) or near (e.g., within 20, 19, 18, 17, 16, 15, 14, 13, 12, 11, 10, 9, 8, 7, 6, 5, 4, 3, 2 or 1 nucleotides of) a nucleic acid sequence having complementarity to the gRNA molecule (e.g., the target sequence of the gRNA molecule) or gRNA molecules introduced into said cells. In embodiments, the indel is a frameshift indel. In embodiments, the cell includes a large deletion, for example a deletion of 1 kb, 2 kb, 3 kb, 4 kb, 5 kb, 6 kb or more. In embodiments, the large deletion comprises nucleic acids disposed between two binding sites for the gRNA molecule or gRNA molecules introduced into said cells.

In an aspect, the invention relates to a population of cells (e.g., as described herein), e.g., a population of HSPCs, which comprises cells which include an indel at or near (e.g., within 20, 19, 18, 17, 16, 15, 14, 13, 12, 11, 10, 9, 8, 7, 6, 5, 4, 3, 2 or 1 nucleotides of) a nucleic acid sequence having complementarity to a gRNA molecule or gRNA molecules, e.g., as described herein, introduced into said cells, e.g., as described herein. In embodiments, the indel is a frameshift indel. In embodiments, the cell population includes cells which comprise a large deletion, for example a deletion of 1 kb, 2 kb, 3 kb, 4 kb, 5 kb, 6 kb or more. In embodiments, the large deletion comprises nucleic acids disposed between two binding sites for the gRNA molecule or gRNA molecules introduced into said cells. In embodiments, 20%-100% of the cells of the population include said large deletion, indel or indels. In embodiments, 30%-100% of the cells of the population include said large deletion, indel or indels. In embodiments, 40%-100% of the cells of the population include said large deletion, indel or indels. In embodiments, 50%-100% of the cells of the population include said large deletion, indel or indels. In embodiments, 60%-100% of the cells of the population include said large deletion, indel or indels. In embodiments, 70%-100% of the cells of the population include said large deletion, indel or indels. In embodiments, 80%-100% of the cells of the population include said large deletion, indel or indels. In embodiments, 90%-100% of the cells of the population include said large deletion, indel or indels. In embodiments, the population of cells retains the ability to differentiate into multiple cell types, e.g., maintains the ability to differentiate into cells of erythroid lineage, e.g., red blood cells, e.g., in a subject, e.g., a human. In embodiments, the edited cells (e.g., HSPC cells, e.g., CD34+ cell, e.g., any subpopulation of CD34+ cell, e.g., as described herein) maintain the ability (and/or do) to proliferate, e.g., in cell culture, e.g., proliferate at least 5-fold, at least 10-fold, at least 15-fold, at least 20-fold, at least 25-fold, at least 30-fold, at least 35-fold, at least 40-fold, at least 45-fold, at least 50-fold or more, e.g., after 1, 2, 3, 4, 5, 6, 7 or more days (e.g., after about 1 or about 2 days) in cell culture, e.g., in a cell culture medium described herein, e.g., a cell culture medium comprising one or more stem cell expanders, e.g., compound 4. In embodiments, the edited and differentiated cells (e.g., red blood cells) maintain the ability to proliferate, e.g., proliferate at least 5-fold, at least 10-fold, at least 15-fold, at least 20-fold, at least 25-fold, at least 30-fold, at least 35-fold, at least 40-fold, at least 45-fold, at least 50-fold or more after 7 days in erythroid differentiation medium (EDM), e.g., as described in the Examples, and/or, proliferate at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, at least 95-fold, at least 100-fold, at least 110-fold, at least 120-fold, at least 130-fold, at least 140-fold, at least 150-fold, at least 200-fold, at least 300-fold, at least 400-fold, at least 500-fold, at least 600-fold, at least 700-fold, at least 800-fold, at least 900-fold, at least 1000-fold, at least 1100-fold, at least 1200-fold, at least 1300-fold, at least 1400-fold, at least 1500-fold or more after 21 days, e.g., in erythroid differentiation medium (EDM), e.g., as described in the Examples or in a subject (e.g., a mammal, e.g., a human).

In an embodiment, the invention provides a population of cells, e.g., CD34+ cells, of which at least 90%, e.g., at least 95%, e.g., at least 98%, of the cells of the population comprise a large deletion or one or more indels, e.g., as described herein. Without being bound by theory, it is believed that introduction of a gRNA molecule or CRISPR system as described herein into a population of cells produces a pattern of indels and/or large deletions in said population, and thus, each cell of the population which comprises an indel and/or large deletion may not exhibit the same indel and/or large deletion. In embodiments, the indel and/or large deletion comprises one or more nucleic acids at or near a site complementary to the targeting domain of a gRNA molecule described herein; wherein said cells maintain the ability to differentiate into cells of an erythroid lineage, e.g., red blood cells; and/or wherein said cells differentiated from the population of cells have an increased level of fetal hemoglobin (e.g., the population has a higher % F cells) relative to cells differentiated from a similar population of unmodified cells. In embodiments, the population of cells has undergone at least a 2-fold expansion ex vivo, e.g., in the media comprising one or more stem cell expanders, e.g., comprising (S)-2-(6-(2-(1H-indol-3-yl)ethylamino)-2-(5-fluoropyridin-3-yl)-9H-purin-9-yl)propan-1-ol. In embodiments, the population of cells has undergone at least a 5-fold expansion ex vivo, e.g., in the media comprising one or more stem cell expanders, e.g., comprising (S)-2-(6-(2-(1H-indol-3-yl)ethylamino)-2-(5-fluoropyridin-3-yl)-9H-purin-9-yl)propan-1-ol.

In embodiments, the indel is less than about 50 nucleotides, e.g., less than about 45, less than about 40, less than about 35, less than about 30 or less than about 25 nucleotides. In embodiments, the indel is less than about 25 nucleotides. In embodiments, the indel is less than about 20 nucleotides. In embodiments, the indel is less than about 15 nucleotides. In embodiments, the indel is less than about 10 nucleotides. In embodiments, the indel is less than about 9 nucleotides. In embodiments, the indel is less than about 9 nucleotides. In embodiments, the indel is less than about 7 nucleotides. In embodiments, the indel is less than about 6 nucleotides. In embodiments, the indel is less than about 5 nucleotides. In embodiments, the indel is less than about 4 nucleotides. In embodiments, the indel is less than about 3 nucleotides. In embodiments, the indel is less than about 2 nucleotides. In any of the aforementioned embodiments, the indel is at least 1 nucleotide. In embodiments, the indel is 1 nucleotide. In embodiments, the large deletion comprises about 1 kb of DNA. In embodiments, the large deletion comprises about 2 kb of DNA. In embodiments, the large deletion comprises about 3 kb of DNA. In embodiments, the large deletion comprises about 4 kb of DNA. In embodiments, the large deletion comprises about 5 kb of DNA. In embodiments, the large deletion comprises about 6 kb of DNA.

In embodiments, a population of cells (e.g., as described herein) comprises a pattern of indels and/or large deletions comprising any 1, 2, 3, 4, 5, or 6 of the most frequently detected indels associated with a CRISPR system comprising a gRNA molecule described herein. In embodiments, the indels and/or large deletions are detected by a method described herein, e.g., by NGS or qPCR.

In an aspect, the cell or population of cells (e.g., as described herein) does not comprise an indel or large deletion at an off-target site, e.g., as detected by a method described herein.

In embodiments, the progeny, e.g., differentiated progeny, e.g., erythroid (e.g., red blood cell) progeny of the cell or population of cells described herein (e.g., derived from a sickle cell disease patient) produce a lower level of sickle beta globin and/or a higher level of gamma globin than unmodified cells. In embodiments, the progeny, e.g., differentiated progeny, e.g., erythroid (e.g., red blood cell) progeny of the cell or population of cells described herein (e.g., derived from a sickle cell disease patient) produce a lower level of sickle beta globin and a higher level of gamma globin than unmodified cells. In embodiments, sickle beta globin is produced at a level at least about 20%, at least about 30%, at least about 40% or at least about 50% lower than unmodified cells. In embodiments, gamma globin is produced at a level at least about 20%, at least about 30%, at least about 40%, at least about 50%, at least about 60% or at least about 70% higher than unmodified cells.

In an aspect, the invention provides a population of modified HSPCs or erythroid cells differentiated from said HSPCs (e.g., differentiated ex vivo or in a patient), e.g., as described herein, wherein at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the cells are F cells. In embodiments, the population of cells contains (or is capable of differentiating, e.g., in vivo, into a population of erythrocytes that contains) a higher percent of F cells than a similar population of cells which have not had a gRNA molecule or gRNA molecules, e.g., as described herein, introduced into said cells. In embodiments, the population of cells has (or is capable of differentiating, e.g., in vivo, into a population of erythrocytes that has) at least a 20% increase, e.g., at least 21% increase, at least 22% increase, at least 23% increase, at least 24% increase, at least 25% increase, at least 26% increase, at least 27% increase, at least 28% increase, or at least 29% increase, in F cells relative to the similar population of cells which have not had a gRNA molecule or gRNA molecules, e.g., as described herein, introduced into said cells. In embodiments, the population of cells has (or is capable of differentiating, e.g., in vivo, into a population of erythrocytes that has) at least a 30% increase, e.g., at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase or at least a 95% increase, in F cells relative to the similar population of cells which have not had a gRNA molecule or gRNA molecules, e.g., as described herein, introduced into said cells. In embodiments, the population of cells has (or is capable of differentiating, e.g., in vivo, into a population of erythrocytes that has) at a 10-90%, a 20%-80%, a 20%-70%, a 20%-60%, a 20%-50%, a 20%-40%, a 20%-30%, a 25%-80%, a 25%-70%, a 25%-60%, a 25%-50%, a 25%-40%, a 25%-35%, a 25%-30%, a 30%-80%, a 30%-70%, a 30%-60%, a 30%-50%, a 30%-40%, or a 30%-35% increase in F cells relative to the similar population of cells which have not had a gRNA molecule or gRNA molecules, e.g., as described herein, introduced into said cells. In embodiments, the population of cells, e.g., as produced by a method described herein, comprises a sufficient number or cells and/or a sufficient increase in % F cells to treat a hemoglobinopathy, e.g., as described herein, e.g., sickle cell disease and/or beta thalassemia, in a patient in need thereof when introduced into said patient, e.g., in a therapeutically effective amount. In embodiments, the increase in F cells is as measured in an erythroid differentiation assay, e.g., as described herein.

In embodiments, including in any of the embodiments and aspects described herein, the invention relates to a cell, e.g., a population of cells, e.g., as modified by any of the gRNA, methods and/or CRISPR systems described herein, comprising F cells that produce at least 6 picograms fetal hemoglobin per cell. In embodiments, the F cells produce at least 7 picograms fetal hemoglobin per cell. In embodiments, the F cells produce at least 8 picograms fetal hemoglobin per cell. In embodiments, the F cells produce at least 9 picograms fetal hemoglobin per cell. In embodiments, the F cells produce at least 10 picograms fetal hemoglobin per cell. In embodiments, the F cells produce an average of between 6.0 and 7.0 picograms, between 7.0 and 8.0, between 8.0 and 9.0, between 9.0 and 10.0, between 10.0 and 11.0, or between 11.0 and 12.0 picograms of fetal hemoglobin per cell.

In embodiments, a cell or population of cells, e.g., as described herein (for example, comprising an indel) (or its progeny), is detectable in the cells of a subject to which it is introduced, for example, remains detectible by detecting the indel, for example, using a method described herein. In embodiments, the cell or population of cells (or its progeny) is detectible in a subject to which it is introduced for at least 10 weeks, at least 14 weeks, at least 16 weeks, at least 18 weeks, at least 20 weeks, at least 30 weeks at least 40 weeks, at least 50 weeks, or longer after said cell or population of cells is introduced into said subject.

In embodiments, one or more indels is detectable in the cells (e.g., the cells, e.g., CD34+ cells, of the bone marrow and/or peripheral blood) of a subject to which the cells or population of cells described herein have been introduced, for example, remains detectible by a method described herein, e.g., NGS. In embodiments, the one or more indels is detectible in the cells (e.g., the cells, e.g., CD34+ cells, of the bone marrow and/or peripheral blood) of a subject to which the cells or population of cells described herein have been introduced for at least 10 weeks, at least 14 weeks, at least 16 weeks, at least 18 weeks, at least 20 weeks, at least 30 weeks at least 40 weeks, at least 50 weeks, or longer after the cell or population of cells described herein is introduced into said subject. In embodiments, the level of detection of said one or more indels does not decrease over time, or decreases by less than 5%, less than 10%, less than 15%, less than 20%, less than 30%, less than 40% or less than 50% (for example relative to the level of indel detection pre-transplant or relative to the level of detection at week 2 post-transplant or at week 8 post-transplant), for example when measured at week 20 post-transplant relative to the level of detection (e.g., percentage of cells comprising the one or more indels) measured pre-transplant or measured at week 2 post-transplant or at week 8 post-transplant.

In embodiments, including in any of the aforementioned embodiments, the cell and/or population of cells of the invention includes, e.g., consists of, cells which do not comprise nucleic acid encoding a Cas9 molecule.

### XVI. Additional ZNF644 Inhibitors and Methods of Use Thereof

As described above, a “ZNF644 inhibitor” refers to a substance that results in a detectably lower expression of ZNF644 gene or ZNF644 protein or lower activity level of ZNF644 proteins as compared to those levels without such substance. In some embodiments, a ZNF644 inhibitor is a small molecule compound (e.g., a small molecule compound that can target ZNF644 for degradation). In some embodiments, a ZNF644 inhibitor is an anti-ZNF644 shRNA. In some embodiments, a ZNF644 inhibitor is an anti-ZNF644 siRNA. In some embodiments, a ZNF644 inhibitor is an anti-ZNF644 ASO. In some embodiments, a ZNF644 inhibitor is an anti-ZNF644 AMO. In some embodiments, a ZNF644 inhibitor is an anti-ZNF644 antisense nucleic acid. In some embodiments, a ZNF644 inhibitor is a composition or a cell or a population of cells (that comprises gRNA molecules described herein) described herein.

Also provided herein are compositions that can reduce ZNF644 gene expression or ZNF644 protein activity. Such compositions include, but are not limited to, small molecule compounds (e.g., small molecule compounds that can target ZNF644 protein for degradation, e.g., through E3 ubiquitin pathway), siRNAs, shRNA, ASOs, miRNAs, AMOs. Exemplary shRNAs include those presented in Table 7.

TABLE 7

| Name | Sequence | SEQ ID NO |
|---|---|---|
| shZNF644-1 | GAAGTCACGTCACTACTTAAA | 2629 |
| shZNF644-2 | GACGAGGTTTACATTCTCCGA | 2630 |
| shZNF644-3 | CAAGGTCAAGATCTGGAAGCA | 2631 |
| shZNF644-4 | CCAGTTTGAATTGGATGTAAA | 2632 |
| shZNF644-5 | GACTGGATTAAGCACTTACAA | 2633 |

One surprising finding by the inventors of the inventions described herein is the linkage between ZNF644 gene expression/protein activity and the hemoglobin F (HbF) production. As demonstrated in the examples and figures, knocking down or knocking out ZNF644 gene in cells significantly increased HbF induction in those cells.

Also provided herein are methods for treating a hemoglobinopathy and by administering to a patient a cell or population of cells or a composition containing such cell or population of cells described herein, or a composition that reduces ZNF644 gene expression and/or ZNF644 protein activity. In aspects, the composition that reduces ZNF644 gene expression and/or ZNF644 protein activity comprises a small molecule compound (e.g., a ZNF644 degrader), siRNA, shRNA, antisense oligonucleotide (ASO), miRNA, anti-microRNA oligonucleotide (AMO) or any combination thereof. In aspects, the hemoglobinopathy is beta-thalassemia or sickle cell disease.

Also provided herein are methods for increasing fetal hemoglobin expression in a mammal by administering to a patient a cell or population of cells or a composition containing such cell or population of cells described herein, or a composition that reduces ZNF644 gene expression and/or ZNF644 protein activity. In aspects, the composition that reduces ZNF644 gene expression and/or ZNF644 protein activity comprises a small molecule compound (e.g., a ZNF644 degrader), siRNA, shRNA, antisense oligonucleotide (ASO), miRNA, anti-microRNA oligonucleotide (AMO) or any combination thereof.

Accordingly, also provided herein are methods for treating a hemoglobinopathy by administering a composition comprising a ZNF644 inhibitor as described herein to a patient. In some embodiments, a ZNF644 inhibitor is a small molecule compound that can target ZNF644 for degradation. In some embodiments, a ZNF644 inhibitor is an anti-ZNF644 shRNA. In some embodiments, a ZNF644 inhibitor is an anti-ZNF644 siRNA. In some embodiments, a ZNF644 inhibitor is an anti-ZNF644 ASO. In some embodiments, a ZNF644 inhibitor is an anti-ZNF644 miRNA. In some embodiments, a ZNF644 inhibitor is an anti-ZNF644 AMO (anti-miRNA oligonucleotides). In some embodiments, a ZNF644 inhibitor is a composition or a cell or a population of cells (that comprises gRNA molecules described herein) described herein.

Also provided herein are methods for increasing fetal hemoglobin expression in a mammal by administering a composition comprising a ZNF644 inhibitor as described herein to the mammal. In some embodiments, a ZNF644 inhibitor is a small molecule compound that can target ZNF644 for degradation. In some embodiments, a ZNF644 inhibitor is an anti-ZNF644 shRNA. In some embodiments, a ZNF644 inhibitor is an anti-ZNF644 siRNA. In some embodiments, a ZNF644 inhibitor is an anti-ZNF644 ASO. In some embodiments, a ZNF644 inhibitor is a composition or a cell or a population of cells (that comprises gRNA molecules described herein) described herein.

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

### EXAMPLES

### Example 1—Exemplary General Methods

### Guide Selection and Design

Initial guide selection was performed in silico using a human reference genome and user defined genomic regions of interest (e.g., a gene, an exon of a gene, non-coding regulatory region, etc.), for identifying PAMs in the regions of interest. For each identified PAM, analyses were performed and statistics reported. gRNA molecules were further selected and rank-ordered based on a number of methods for determining efficiency and efficacy, e.g., as described herein. This example provides the experimental details for procedures that can be used to assay the CRISPR systems, gRNAs and other aspects of the invention described herein. Any modifications to these general procedures that were employed in a particular experiment are noted in that example.

### Next-Generation Sequencing (NGS) and Analysis for On-Target Cleavage Efficiency and Indel Formation

To determine the efficiency of editing (e.g., cleaving) the target location in the genome, deep sequencing is utilized to identify the presence of insertions and deletions introduced by non-homologous end joining.

In summary PCR primers are designed around the target site, and the genomic area of interest are PCR amplified in edited and unedited samples. Resulting amplicons are converted into Illumina sequencing libraries and sequenced. Sequencing reads are aligned to the human genome reference and subjected to variant calling analysis allowing us to the determine sequence variants and their frequency at the target region of interest. Data are subjected to various quality filters and known variants or variants identified only in the unedited samples were excluded. The editing percentage is defined as the percentage of all insertions or deletions events occurring at the on-target site of interest (i.e. insertion and deletion reads at the on-target site over the total number of reads (wild type and mutant reads) at on-target site.

### RNP Generation

The addition of crRNA and tracrRNA to Cas9 protein results in the formation of the active Cas9 ribonucleoprotein complex (RNP), which mediates binding to the target region specified by the crRNA and specific cleavage of the targeted genomic DNA. This complex is formed by loading tracrRNA and crRNA into Cas9, which is believed to cause conformational changes to Cas9 allowing it to bind and cleave dsDNA.

The crRNA and tracrRNA are separately denatured at 95° C. for 2 minutes, and allowed to come to room temperature. Cas9 protein (10 mg/ml) was added to 5×CCE buffer (20 mM HEPES, 100 mM KCl, 5 mM MgCl₂, 1 mM DTT, 5% glycerol), to which tracrRNA and the various crRNAs are then added (in separate reactions) and incubated at 37° C. for 10 minutes, thereby forming the active RNP complex. The complex is delivered by electroporation and other methods into a wide variety of cells, including HEK-293 and CD34+ hematopoietic cells.

### Delivery of RNPs to CD34+ HSCs

Cas9 RNPs were delivered into CD34+ HSCs.

CD34+ HSCs are thawed and cultured (at ˜500,000 cells/ml) overnight in StemSpan SFEM (StemCell Technologies) media with IL6, SCF, TPO, Flt3L and Pen/Strep added. Roughly 90,000 cells were aliquoted and pelleted per each RNP delivery reaction. The cells are then resuspended in 60 ul P3 nucleofection buffer (Lonza), to which active RNP was subsequently added. The HSCs are then electroporated (e.g., nucleofected using program CA-137 on a Lonza Nucleofector) in triplicate (20 μL/electroporation). Immediately following electroporation, StemSpan SFEM media (with IL12, SCF, TPO, Flt3L and Pen/Strep) is added to the HSCs, which is cultured for at least 24 hours. HSCs are then harvested and subjected to T7E1, NGS, and/or surface marker expression analyses.

### HSC Functional Assay

CD34+ HSCs may be assayed for stem cell phenotype using known techniques such as flow cytometry or the in vitro colony forming assay. By way of example, cells are assayed by the in vitro colony forming assay (CFC) using the Methocult H4034 Optimum kit (StemCell Technologies) using the manufacturer's protocol. Briefly, 500-2000 CD34+ cells in <=100 ul volume are added to 1-1.25 ml methocult. The mixture is vortexed vigorously for 4-5 seconds to mix thoroughly, then allowed to rest at room temperature for at least 5 minutes. Using a syringe, 1-1.25 ml of MethoCult+ cells is transferred to a 35 mm dish or well of a 6-well plate. Colony number and morphology is assessed after 12-14 days as per the manufacturer's protocol.

### In Vivo Xeno-Transplantation

HSCs are functionally defined by their ability to self-renew and for multi-lineage differentiation. This functionality can only be assessed in vivo. The gold-standard for determining human HSC function is through xeno-transplantation into the NOD-SCID gamma mouse (NSG) that through a series of mutations is severely immunocompromised and thus can act as a recipient for human cells. HSCs following editing were transplanted into NSG mice to validate that the induced edit does not impact HSC function. Periodic peripheral blood analysis is used to assess human chimerism and lineage development and secondary transplantation following 20 weeks is used to establish the presence of functional HSCs, as described more fully in these examples.

### Example 2—Loss of ZNF644 Induces Fetal Hemoglobin Expression in mPB CD34+ Derived Erythroid Cells

### Materials and Methods

### Cell Culture

HEK293T cells were maintained in DMEM high glucose complete media with sodium pyrovate, non-essential amino acids, 10% FBS, 1× L-glutamine (2 mM), 1% pen/strep (100 U/ml), 1× HEPES (25 mM). Unless disclosed otherwise, all reagents for culturing HEK293T cells were obtained from Invitrogen™.

Mobilized peripheral blood (mPB) CD34+ cells (AllCells, LLC) were maintained in StemSpanT^{M} serum-free expansion media (SFEM) (STEMCELL Technologies Inc.) supplemented with 50 ng/mL each of rhTPO, rhIL-6, rhFLT3L, rhSCF for 2-3 days prior to shRNA transduction or targeted ribonucleoprotein (RNP) electroporation targeting ZNF644. All cytokines were obtained from Peprotech®, Inc. Cell cultures were maintained at 37° C. and 5% CO₂ in a humidified tissue culture incubator.

Generation of shRNA Lentiviral Clones Targeting ZNF644

5′-phosphorylated sense and anti-sense complementary single-stranded DNA oligos of the respective shRNA against ZNF644 were synthesized by Integrated DNA Technologies, Inc. (IDT). Each DNA oligonucleotide was designed with PmeI/AscI restriction overhangs on 5′- and 3′-ends, respectively, for subsequent compatible ligation into the lentiviral vector backbone. Equimolar of each of the complementary oligonucleotides were annealed in NEB Buffer 2 (New England Biolabs® Inc.) by heating on a heating block at 98° C. for 5 minutes followed by cooling to room temperature on the bench top. Annealed double-stranded DNA oligonucleotides were ligated into pHAGE lentiviral backbone digested with PmeI/AscI using T4 DNA ligase kit (New England Biolabs). Ligation reactions were transformed into chemically competent Stb13 cells (Invitrogen™) according to the manufacturer's protocol. Positive clones were verified using mU6 sequencing primer (5′-ctacattttacatgatagg-3′) (SEQ ID NO: 3206) and plasmids were purified by Alta Biotech LLC.

Lentivirus particles for the respective shRNA constructs were generated by co-transfection of HEK293T cells with pCMV-dR8.91 and pCMV-VSV-G expressing envelope plasmid using Lipofectamine 3000 reagent in 150 mm tissue culture dish format as per manufacturer's instructions (Invitrogen™). Lentivirus supernatant was harvested 48 hours after co-transfection, filtered through a 0.45 μm filter (Millipore) and concentrated using Amicon Ultra 15 with Ultracel-100 membrane (Millipore). Infectious units of each of the lentivirus particle was determined by flow cytometry using eGFP expression as marker of transduction after serial dilution and infection of HEK293T cells.

Lentiviral shRNA Transduction and FACS Sorting of mPB CD34+ Cells

mPB CD34+ transduction was performed on retronectin coated non-tissue culture treated 96 well-flat bottom plates (Corning, Inc.). Briefly, TC plates were coated with 100 μL of RetroNectin® (1 μg/mL) (TAKARABIO, Inc.), sealed and incubated at 4° C. overnight. RetroNectin® was then removed and plates were incubated with BSA (bovine serum albumin) (1%) in PBS for 30 minutes at room temperature. Subsequently, BSA (bovine serum albumin) was aspirated and replaced with 100 μL of lentiviral concentrate and centrifuged at 2000×g for 2 hours at room temperature. Next, residual supernatant was gently pipetted out and ready for transductions of mPB CD34+ cells. Ten thousand cells were plated in 150 μL of StemSpan™ Serum-free Expansion Medium (SFEM) supplemented with 50 ng/mL each of rhTPO, rhIL-6, rhFLT3L, rhSCF to initiate transduction. Cells were cultured for 72 hours prior to assessing transduction efficiencies using eGFP expression as a marker.

eGFP-positive cells were sorted on an FACSAria™ III (BD Biosciences). Briefly, the transduced mPB CD34+ cell population was washed and re-suspended with FACS buffer containing 1× Hank's buffered saline solution, EDTA (1 mM) and FBS (2%). Sorted eGFP-positive cells were used for the erythroid differentiation assay.

### Targeting CRISPR Knockout of ZNF644

Alt-R CRISPR-Cas9 crRNA and tracrRNA (5′-AGCAUAGCAAGUUAAAAUAAGGCUAGUCCGUUAUCAACUUGAAAAAGUGGCACCGAG UCGGUGCUUU-3′; SEQ ID NO: 3156) were purchased from Integrated DNA Technologies, Inc. Equimolar tracrRNA was annealed with ZNF644 targeting crRNA (Table 8) in Tris buffer (10 mM, pH 7.5) by heating at 95° C. for 5 minutes followed by cooling to room temperature using a polymerase chain reaction (PCR) machine (Bio-Rad). Subsequently, a ribonucleoprotein (RNP) complex was generated by mixing annealed tracrRNA:crRNA with 6 μg of Cas9 at 37° C. for 5 minutes in 1× buffer containing HEPES (100 mM), KCl (50 mM), MgCl₂ (2.5 mM), glycerol (0.03%), DTT (1 mM) and Tris pH 7.5 (2 mM).

Electroporation of the RNP complex was performed on a 4D-Nucleofector™ (Lonza) as per manufacturer's recommendation. Briefly, 50,000 mPB CD34+ cells resuspended in Primary Cell P3 Buffer with supplement (Lonza) were pre-mixed with 5 μL of RNP complex per well in nucleocuvettes and incubated for 5 minutes at room temperature. Subsequently, the mixture was electroporated using the CM-137 program. Cells were cultured for 72 hours post-RNP electroporation before initiating erythroid differentiation.

TABLE 8

| | | Target | | SEQ |
|---|---|---|---|---|
| | Sequence | genomic | | ID |
| Name | (5' to 3') | region | Strand | NO |
| rg_0111 | ACGGAGGCTA | random | | 3108 |
| | AGCGTCGCAA | guide, | | |
| | | non- | | |
| | | targeting | | |
| ZNF644_1 | AACGCTAATC | chr1: | − | 2634 |
| | TTCCACGGAC | 90916895- | | |
| | | 90916914 | | |
| ZNF644_2 | TCGAGGACCC | chr1: | − | 2635 |
| | TTGTCTGTTC | 90916953- | | |
| | | 90916972 | | |
| ZNF644_3 | ACAAGGGTCC | chr1: | + | 2636 |
| | TCGAAAGACT | 90916959- | | |
| | | 90916978 | | |
| ZNF644_4 | TAAACCTCGT | chr1: | + | 2637 |
| | CAGCACCATG | 90918067- | | |
| | | 90918086 | | |
| ZNF644_5 | CGAAGCAGCT | chr1: | − | 2638 |
| | TCATAGAACC | 90916794- | | |
| | | 90916813 | | |
| ZNF644_6 | CTTACAATCT | chr1: | + | 2639 |
| | AAGGCTAAAA | 90918141- | | |
| | | 90918160 | | |
| ZNF644_7 | TTGTAAACGC | chr1: | − | 2640 |
| | TAATCTTCCA | 90916900- | | |
| | | 90916919 | | |
| ZNF644_8 | AGGTTTACAT | chr1: | − | 2641 |
| | TCTCCGATGC | 90918054- | | |
| | | 90918073 | | |

Erythroid Differentiation of shRNA Transduced or RNP Electroporated mPB CD34+ Cells

Erythroid differentiation was initiated by plating 8,000 RNP-electroporated or FACS sorted eGFP+ mPB CD34+ cells per well in 96-well tissue culture plates. Base differentiation media consists of IMDM (Iscove's Modified Dulbecco's Medium), human AB serum (5%), transferrin (330 μg/mL), Insulin (10 μg/mL) and Heparin (2 IU/mL). Differentiation media was supplemented with rhSCF (100 ng/mL), rhIL-3 (10 ng/mL), rhEPO (2.5 U/mL) and hydrocortisone (1 μM). After 4 days of differentiation, the cells were split (1:4) in fresh media to maintain optimal growth density. Cells were cultured for additional 3 days and utilized for assessment of fetal hemoglobin (HbF) expression.

### HbF Intracellular Staining

One hundred thousand cells were aliquoted into U-bottom 96-well plates and stained for 20 min in the dark with diluted LIVE/DEAD fixable violet viability dye as per manufacturer's recommendation (Invitrogen). Cells were washed with FACS staining buffer and subsequently stained with anti-CD71-BV711 (BD Biosciences) and anti-CD235a-APC (BD Biosciences) for 20 mins in the dark. After two rounds of washes with three volumes of 1×PBS, cells were fixed and permeabilized with 1×BD Cytofix/Cytoperm (BD Biosciences) for 30 minutes at room temperature in the dark. Subsequently, cells were washed twice with three volumes of 1× Perm/wash buffer (BD Biosciences). Anti-HbF-FITC (ThermoScientific) was diluted (1:25) in 1× perm/wash buffer, added to permeabilized cells and incubated for 30 minutes at room temperature in the dark. Next, cells were washed twice with three volumes of 1× perm/washbuffer and analyzed by flow cytometry using LSR Fortessa (BD Biosciences). Data was analyzed with FlowJo software.

### Results

### ZNF644 Knockdown and KO Upregulate HbF Protein

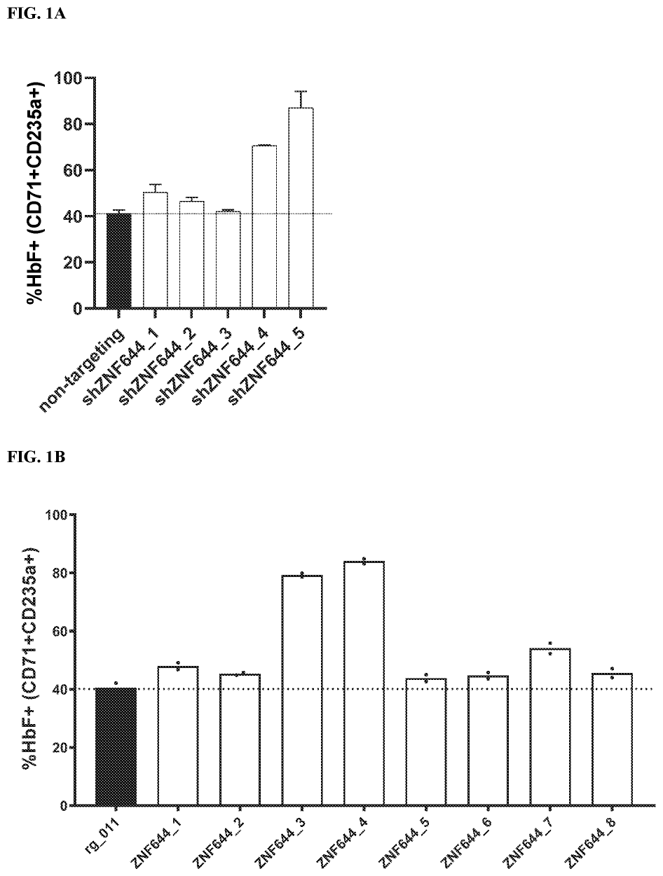

In order to validate whether ZNF644 is a negative regulator of HbF expression, shRNA and CRISPR-Cas9-mediated knockdown and knockout functional genetics approaches were employed. mPB CD34+ cells were treated with shRNA or CRISPR-Cas9 reagents and erythroid differentiated for 7 days prior to flow cytometry analysis. Targeted knockdown of ZNF644 transcript results in up to 92% HbF+ cells compared to 42% for the negative control scrambled shRNA (FIG. 1A). Error bars represent standard deviation of one biological replicate with two technical replicates each. CRISPR/Cas9-mediated targeted loss of ZNF644 results in up to 85% HbF+ cells compared to 42% for random guide crRNA. ZNF644_3 and ZNF644_4 gRNAs achieved 63% and 94% editing efficiency, respectively, determined by Sanger sequencing followed by TIDE analysis. Data represents one biological sample with two technical replicates (FIG. 1B). To summarize, modulation (e.g. inhibiting) of ZNF644 by shRNA knockdown (demonstrated using five different shRNA sequences) or CRISPR knockout (demonstrated using eight different gRNA sequences) induces fetal hemoglobin expression in human primary erythroid cells. These data provide genetic evidence that ZNF644 is a regulator of fetal hemoglobin expression and represents a novel target for the treatment of sickle cell disease and beta-thalassemia.

### Tracking of Indels by Decomposition (TIDE)

Editing efficiency was determined by TIDE analysis (Brinkman, E. K., et al. Easy quantitative assessment of genome editing by sequence trace decomposition. Nucleic Acids Res. 42, e168 (2014)). Briefly, gDNA was extracted post-electroporation with 1×10⁴ cells/1 QuickExtract DNA extraction solution (Epicentre). Genomic regions containing CRISPR-targeted sites were PCR-amplified with Q5 high-fidelity DNA polymerase (New England Biolabs), purified with the QiAquick PCR purification kit (Qiagen) or ZR-96 DNA Clean & Concentrator-5 (Zymto Research) and sequenced by Sanger sequencing. Primer sequences are as follows:

ZNF644_3F:
(SEQ ID NO: 3185)
TTAAGGAATGCTAAACTGGGGA
ZNF644_3R:
(SEQ ID NO: 3186)
GCTTCGGCCATTAGTAGAGAAA
ZNF644_4F:
(SEQ ID NO: 3187)
ATGCTTCAGGGAATAGGGAAAT
ZNF644_4R:
(SEQ ID NO: 3188)
CACCTGGCCAAGCTACTTTAAT

To the extent there are any discrepancies between any sequence listing and any sequence recited in the specification, the sequence recited in the specification should be considered the correct sequence. Unless otherwise indicated, all genomic locations are according to hg38.

TABLE 1

| | | | | | gRNA_ | | |
|---|---|---|---|---|---|---|---|
| SEQ | | | target_ | target_ | target_ | | |
| ID | target_ | target_ | region_ | region | site_ | gRNA Targeting | |
| NO | gene_id | symbol | name | coordinates | coordinates | Domain | strand |
| 1 | 84146 | ZNF644 | promoter1.1 | chr1: | chr1: | AAAUACAGGAUGAUAGUUCG | + |
| | | | | 91023900- | 91024058- | | |
| | | | | 91024866 | 91024077 | | |
| 2 | 84146 | ZNF644 | promoter1.2 | chr1: | chr1: | UGAUGAAACGCUGUACAAAA | + |
| | | | | 91022273- | 91023767- | | |
| | | | | 91023899 | 91023786 | | |
| 3 | 84146 | ZNF644 | promoter1.2 | chr1: | chr1: | GUGAGACAAGUUCCUGAUCA | − |
| | | | | 91022273- | 91023636- | | |
| | | | | 91023899 | 91023655 | | |
| 4 | 84146 | ZNF644 | promoter1.2 | chr1: | chr1: | UUUGUCUGCACAUUAGUCAG | − |
| | | | | 91022273- | 91023580- | | |
| | | | | 91023899 | 91023599 | | |
| 5 | 84146 | ZNF644 | promoter1.2 | chr1: | chr1: | GGACAUUCUCAGUUUACUAU | + |
| | | | | 91022273- | 91023464- | | |
| | | | | 91023899 | 91023483 | | |
| 6 | 84146 | ZNF644 | promoter1.2 | chr1: | chr1: | AAUGGGGGAGGGGUGCUCUU | + |
| | | | | 91022273- | 91023411- | | |
| | | | | 91023899 | 91023430 | | |
| 7 | 84146 | ZNF644 | promoter1.2 | chr1: | chr1: | UAUUUGCAGUCAAUGGGGGA | + |
| | | | | 91022273- | 91023400- | | |
| | | | | 91023899 | 91023419 | | |
| 8 | 84146 | ZNF644 | promoter1.2 | chr1: | chr1: | AUAUUUGCAGUCAAUGGGGG | + |
| | | | | 91022273- | 91023399- | | |
| | | | | 91023899 | 91023418 | | |
| 9 | 84146 | ZNF644 | promoter1.2 | chr1: | chr1: | CGUAUAUAUUUGCAGUCAAU | + |
| | | | | 91022273- | 91023394- | | |
| | | | | 91023899 | 91023413 | | |
| 10 | 84146 | ZNF644 | promoter1.2 | chr1: | chr1: | ACGUAUAUAUUUGCAGUCAA | + |
| | | | | 91022273- | 91023393- | | |
| | | | | 91023899 | 91023412 | | |
| 11 | 84146 | ZNF644 | promoter1.2 | chr1: | chr1: | UCGAAGUCAUACAGUUCUUU | + |
| | | | | 91022273- | 91023308- | | |
| | | | | 91023899 | 91023327 | | |
| 12 | 84146 | ZNF644 | promoter1.2 | chr1: | chr1: | UGUAACCCAAUGAGACAAAG | + |
| | | | | 91022273- | 91023270- | | |
| | | | | 91023899 | 91023289 | | |
| 13 | 84146 | ZNF644 | promoter1.2 | chr1: | chr1: | UGCUGAAGGCAUCAUUCCAU | − |
| | | | | 91022273- | 91023125- | | |
| | | | | 91023899 | 91023144 | | |
| 14 | 84146 | ZNF644 | promoter1.2 | chr1: | chr1: | UUGUCAAAUAAAUGCAGUUA | − |
| | | | | 91022273- | 91023082- | | |
| | | | | 91023899 | 91023101 | | |
| 15 | 84146 | ZNF644 | promoter1.2 | chr1: | chr1: | CGAAUGUCAAUAACAUCUUG | − |
| | | | | 91022273- | 91023049- | | |
| | | | | 91023899 | 91023068 | | |
| 16 | 84146 | ZNF644 | promoter1.2 | chr1: | chr1: | UCAAUAACAUCUUGAGGGUU | − |
| | | | | 91022273- | 91023043- | | |
| | | | | 91023899 | 91023062 | | |
| 17 | 84146 | ZNF644 | promoter1.2 | chr1: | chr1: | CUGGUCCCGCCUUAAUUAUU | + |
| | | | | 91022273- | 91022932- | | |
| | | | | 91023899 | 91022951 | | |
| 18 | 84146 | ZNF644 | promoter1.2 | chr1: | chr1: | GUAGAUUUCUCAAGAACUCU | − |
| | | | | 91022273- | 91022913- | | |
| | | | | 91023899 | 91022932 | | |
| 19 | 84146 | ZNF644 | promoter1.2 | chr1: | chr1: | ACAUGUUAGCAGUUUCUCGU | − |
| | | | | 91022273- | 91022880- | | |
| | | | | 91023899 | 91022899 | | |
| 20 | 84146 | ZNF644 | promoter1.2 | chr1: | chr1: | GACUUCACCACAGGAUCCUG | + |
| | | | | 91022273- | 91022811- | | |
| | | | | 91023899 | 91022830 | | |
| 21 | 84146 | ZNF644 | promoter1.2 | chr1: | chr1: | AAUCAGUCCCUAGAGGGUUC | + |
| | | | | 91022273- | 91022769- | | |
| | | | | 91023899 | 91022788 | | |
| 22 | 84146 | ZNF644 | promoter1.2 | chr1: | chr1: | UCAGAGAAUCAGUCCCUAGA | + |
| | | | | 91022273- | 91022763- | | |
| | | | | 91023899 | 91022782 | | |
| 23 | 84146 | ZNF644 | promoter1.2 | chr1: | chr1: | UUCAGAGAAUCAGUCCCUAG | + |
| | | | | 91022273- | 91022762- | | |
| | | | | 91023899 | 91022781 | | |
| 24 | 84146 | ZNF644 | promoter1.2 | chr1: | chr1: | UCUAGGGACUGAUUCUCUGA | − |
| | | | | 91022273- | 91022763- | | |
| | | | | 91023899 | 91022782 | | |
| 25 | 84146 | ZNF644 | promoter1.2 | chr1: | chr1: | CUAGGGACUGAUUCUCUGAA | − |
| | | | | 91022273- | 91022762- | | |
| | | | | 91023899 | 91022781 | | |
| 26 | 84146 | ZNF644 | promoter1.2 | chr1: | chr1: | CACGACACUUUCAGUAAUGC | − |
| | | | | 91022273- | 91022692- | | |
| | | | | 91023899 | 91022711 | | |
| 27 | 84146 | ZNF644 | promoter1.2 | chr1: | chr1: | ACGACACUUUCAGUAAUGCA | − |
| | | | | 91022273- | 91022691- | | |
| | | | | 91023899 | 91022710 | | |
| 28 | 84146 | ZNF644 | promoter1.2 | chr1: | chr1: | AGUCAAACUGGAGCAAACCU | − |
| | | | | 91022273- | 91022651- | | |
| | | | | 91023899 | 91022670 | | |
| 29 | 84146 | ZNF644 | promoter1.2 | chr1: | chr1: | CCUGCCCUGAAAGGUGGCCG | + |
| | | | | 91022273- | 91022631- | | |
| | | | | 91023899 | 91022650 | | |
| 30 | 84146 | ZNF644 | promoter1.2 | chr1: | chr1: | CCCGGACAGCCUGCCCUGAA | + |
| | | | | 91022273- | 91022622- | | |
| | | | | 91023899 | 91022641 | | |
| 31 | 84146 | ZNF644 | promoter1.2 | chr1: | chr1: | CCUCGGCCACCUUUCAGGGC | − |
| | | | | 91022273- | 91022634- | | |
| | | | | 91023899 | 91022653 | | |
| 32 | 84146 | ZNF644 | promoter1.2 | chr1: | chr1: | ACCUUUCAGGGCAGGCUGUC | − |
| | | | | 91022273- | 91022626- | | |
| | | | | 91023899 | 91022645 | | |
| 33 | 84146 | ZNF644 | promoter1.2 | chr1: | chr1: | CCUUUCAGGGCAGGCUGUCC | − |
| | | | | 91022273- | 91022625- | | |
| | | | | 91023899 | 91022644 | | |
| 34 | 84146 | ZNF644 | promoter1.2 | chr1: | chr1: | AGUGUGACAUCUCAGUUACC | + |
| | | | | 91022273- | 91022604- | | |
| | | | | 91023899 | 91022623 | | |
| 35 | 84146 | ZNF644 | promoter1.2 | chr1: | chr1: | GAUUGUUGGGAGUUGCUUGA | + |
| | | | | 91022273- | 91022566- | | |
| | | | | 91023899 | 91022585 | | |
| 36 | 84146 | ZNF644 | promoter1.2 | chr1: | chr1: | UCUUAAGGGGGCAGAUUGUU | + |
| | | | | 91022273- | 91022553- | | |
| | | | | 91023899 | 91022572 | | |
| 37 | 84146 | ZNF644 | promoter1.2 | chr1: | chr1: | AUCUUAAGGGGGCAGAUUGU | + |
| | | | | 91022273- | 91022552- | | |
| | | | | 91023899 | 91022571 | | |
| 38 | 84146 | ZNF644 | promoter1.2 | chr1: | chr1: | GAAGGUUUUAGGAUCUUAAG | + |
| | | | | 91022273- | 91022540- | | |
| | | | | 91023899 | 91022559 | | |
| 39 | 84146 | ZNF644 | promoter1.2 | chr1: | chr1: | GGAAGGUUUUAGGAUCUUAA | + |
| | | | | 91022273- | 91022539- | | |
| | | | | 91023899 | 91022558 | | |
| 40 | 84146 | ZNF644 | promoter1.2 | chr1: | chr1: | GGGAAGGUUUUAGGAUCUUA | + |
| | | | | 91022273- | 91022538- | | |
| | | | | 91023899 | 91022557 | | |
| 41 | 84146 | ZNF644 | promoter1.2 | chr1: | chr1: | AAUGGCAGAGGGAAGGUUUU | + |
| | | | | 91022273- | 91022529- | | |
| | | | | 91023899 | 91022548 | | |
| 42 | 84146 | ZNF644 | promoter1.2 | chr1: | chr1: | CAAUCACAACUGUGGAGUAA | + |
| | | | | 91022273- | 91022511- | | |
| | | | | 91023899 | 91022530 | | |
| 43 | 84146 | ZNF644 | promoter1.2 | chr1: | chr1: | CAUUACUCCACAGUUGUGAU | − |
| | | | | 91022273- | 91022513- | | |
| | | | | 91023899 | 91022532 | | |
| 44 | 84146 | ZNF644 | promoter1.2 | chr1: | chr1: | UUGCUGUUUUCCUCAACAAG | − |
| | | | | 91022273- | 91022484- | | |
| | | | | 91023899 | 91022503 | | |
| 45 | 84146 | ZNF644 | promoter1.2 | chr1: | chr1: | UUUUUGCACGCUCGUGAUCG | − |
| | | | | 91022273- | 91022448- | | |
| | | | | 91023899 | 91022467 | | |
| 46 | 84146 | ZNF644 | promoter1.2 | chr1: | chr1: | CUUUAAGGUGGAACAGGCCC | + |
| | | | | 91022273- | 91022414- | | |
| | | | | 91023899 | 91022433 | | |
| 47 | 84146 | ZNF644 | promoter1.2 | chr1: | chr1: | UAGGCCCUUUAAGGUGGAAC | + |
| | | | | 91022273- | 91022408- | | |
| | | | | 91023899 | 91022427 | | |
| 48 | 84146 | ZNF644 | promoter1.2 | chr1: | chr1: | CAUGCCUAGGCCCUUUAAGG | + |
| | | | | 91022273- | 91022402- | | |
| | | | | 91023899 | 91022421 | | |
| 49 | 84146 | ZNF644 | promoter1.2 | chr1: | chr1: | CUGGGCCUGUUCCACCUUAA | − |
| | | | | 91022273- | 91022416- | | |
| | | | | 91023899 | 91022435 | | |
| 50 | 84146 | ZNF644 | promoter1.2 | chr1: | chr1: | UGGGCCUGUUCCACCUUAAA | − |
| | | | | 91022273- | 91022415- | | |
| | | | | 91023899 | 91022434 | | |
| 51 | 84146 | ZNF644 | promoter1.2 | chr1: | chr1: | AGGCAUGCCUAGGCCCUUUA | + |
| | | | | 91022273- | 91022399- | | |
| | | | | 91023899 | 91022418 | | |
| 52 | 84146 | ZNF644 | promoter1.2 | chr1: | chr1: | UGUUCCACCUUAAAGGGCCU | − |
| | | | | 91022273- | 91022409- | | |
| | | | | 91023899 | 91022428 | | |
| 53 | 84146 | ZNF644 | promoter1.2 | chr1: | chr1: | UAGGCACGCUCCAAAUAUGG | + |
| | | | | 91022273- | 91022348- | | |
| | | | | 91023899 | 91022367 | | |
| 54 | 84146 | ZNF644 | promoter1.2 | chr1: | chr1: | AAGUAGGCACGCUCCAAAUA | + |
| | | | | 91022273- | 91022345- | | |
| | | | | 91023899 | 91022364 | | |
| 55 | 84146 | ZNF644 | promoter1.2 | chr1: | chr1: | UAUUUGGAGCGUGCCUACUU | − |
| | | | | 91022273- | 91022345- | | |
| | | | | 91023899 | 91022364 | | |
| 56 | 84146 | ZNF644 | promoter1.2 | chr1: | chr1: | AUUUGGAGCGUGCCUACUUU | − |
| | | | | 91022273- | 91022344- | | |
| | | | | 91023899 | 91022363 | | |
| 57 | 84146 | ZNF644 | promoter1.2 | chr1: | chr1: | GGAGGUUCCCGCCCCAAAGU | + |
| | | | | 91022273- | 91022329- | | |
| | | | | 91023899 | 91022348 | | |
| 58 | 84146 | ZNF644 | promoter1.2 | chr1: | chr1: | UUUGGAGCGUGCCUACUUUG | − |
| | | | | 91022273- | 91022343- | | |
| | | | | 91023899 | 91022362 | | |
| 59 | 84146 | ZNF644 | promoter1.2 | chr1: | chr1: | GGAGCGUGCCUACUUUGGGG | − |
| | | | | 91022273- | 91022340- | | |
| | | | | 91023899 | 91022359 | | |
| 60 | 84146 | ZNF644 | promoter1.2 | chr1: | chr1: | GAGCGUGCCUACUUUGGGGC | − |
| | | | | 91022273- | 91022339- | | |
| | | | | 91023899 | 91022358 | | |
| 61 | 84146 | ZNF644 | promoter1.2 | chr1: | chr1: | CUUUGGGGGGGGAACCUCCU | − |
| | | | | 91022273- | 91022328- | | |
| | | | | 91023899 | 91022347 | | |
| 62 | 84146 | ZNF644 | promoter1.2 | chr1: | chr1: | UUUGGGGGGGGAACCUCCUU | − |
| | | | | 91022273- | 91022327- | | |
| | | | | 91023899 | 91022346 | | |
| 63 | 84146 | ZNF644 | promoter1.2 | chr1: | chr1: | CGCUGACCUAAUCACUCCCA | + |
| | | | | 91022273- | 91022308- | | |
| | | | | 91023899 | 91022327 | | |
| 64 | 84146 | ZNF644 | promoter1.2 | chr1: | chr1: | GGGAGUGAUUAGGUCAGCGC | − |
| | | | | 91022273- | 91022307- | | |
| | | | | 91023899 | 91022326 | | |
| 65 | 84146 | ZNF644 | promoter1.2 | chr1: | chr1: | GGAGUGAUUAGGUCAGCGCU | − |
| | | | | 91022273- | 91022306- | | |
| | | | | 91023899 | 91022325 | | |
| 66 | 84146 | ZNF644 | promoter1.2 | chr1: | chr1: | CGGGUGCCAGAAACCAAACA | + |
| | | | | 91022273- | 91022272- | | |
| | | | | 91023899 | 91022291 | | |
| 67 | 84146 | ZNF644 | promoter1.2 | chr1: | chr1: | AGGUGACCUUGUUUGGUUUC | − |
| | | | | 91022273- | 91022281- | | |
| | | | | 91023899 | 91022300 | | |
| 68 | 84146 | ZNF644 | exon_01_nc.1 | chr1: | chr1: | AAACAGUGCACCACUGUUCU | + |
| | | | | 91022256- | 91022252- | | |
| | | | | 91022272 | 91022271 | | |
| 69 | 84146 | ZNF644 | exon_01_nc.1 | chr1: | chr1: | UUUCUGGCACCCGAGAACAG | − |
| | | | | 91022256- | 91022265- | | |
| | | | | 91022272 | 91022284 | | |
| 70 | 84146 | ZNF644 | exon_01_nc.2 | chr1: | chr1: | AAUUAGACAAACGAGGGCAG | + |
| | | | | 91022082- | 91022229- | | |
| | | | | 91022255 | 91022248 | | |
| 71 | 84146 | ZNF644 | exon_01_nc.2 | chr1: | chr1: | UGGGCCAAUUAGACAAACGA | + |
| | | | | 91022082- | 91022223- | | |
| | | | | 91022255 | 91022242 | | |
| 72 | 84146 | ZNF644 | exon_01_nc.2 | chr1: | chr1: | CUGGGCCAAUUAGACAAACG | + |
| | | | | 91022082- | 91022222- | | |
| | | | | 91022255 | 91022241 | | |
| 73 | 84146 | ZNF644 | exon_01_nc.2 | chr1: | chr1: | UUCGCGCUGGUGGAGCGUCU | + |
| | | | | 91022082- | 91022204- | | |
| | | | | 91022255 | 91022223 | | |
| 74 | 84146 | ZNF644 | exon_01_nc.2 | chr1: | chr1: | CUUCGCGCUGGUGGAGCGUC | + |
| | | | | 91022082- | 91022203- | | |
| | | | | 91022255 | 91022222 | | |
| 75 | 84146 | ZNF644 | exon_01_nc.2 | chr1: | chr1: | CCCGGGAAGCUUCGCGCUGG | + |
| | | | | 91022082- | 91022194- | | |
| | | | | 91022255 | 91022213 | | |
| 76 | 84146 | ZNF644 | exon_01_nc.2 | chr1: | chr1: | GCUCCCGGGAAGCUUCGCGC | + |
| | | | | 91022082- | 91022191- | | |
| | | | | 91022255 | 91022210 | | |
| 77 | 84146 | ZNF644 | exon_01_nc.2 | chr1: | chr1: | UCCACCAGCGCGAAGCUUCC | − |
| | | | | 91022082- | 91022198- | | |
| | | | | 91022255 | 91022217 | | |
| 78 | 84146 | ZNF644 | exon_01_nc.2 | chr1: | chr1: | CCACCAGCGCGAAGCUUCCC | − |
| | | | | 91022082- | 91022197- | | |
| | | | | 91022255 | 91022216 | | |
| 79 | 84146 | ZNF644 | exon_01_nc.2 | chr1: | chr1: | GCUUCCCGGGAGCUCCUCGC | − |
| | | | | 91022082- | 91022184- | | |
| | | | | 91022255 | 91022203 | | |
| 80 | 84146 | ZNF644 | exon_01_nc.2 | chr1: | chr1: | GCCUCCAGCCCCGGUUCCCA | + |
| | | | | 91022082- | 91022107- | | |
| | | | | 91022255 | 91022126 | | |
| 81 | 84146 | ZNF644 | exon_01_nc.2 | chr1: | chr1: | CGCGCCUGCGCCGUGGGAAC | − |
| | | | | 91022082- | 91022120- | | |
| | | | | 91022255 | 91022139 | | |
| 82 | 84146 | ZNF644 | exon_01_nc.2 | chr1: | chr1: | GCGCCUGCGCCGUGGGAACC | − |
| | | | | 91022082- | 91022119- | | |
| | | | | 91022255 | 91022138 | | |
| 83 | 84146 | ZNF644 | exon_01_nc.2 | chr1: | chr1: | CGCCUGCGCCGUGGGAACCG | − |
| | | | | 91022082- | 91022118- | | |
| | | | | 91022255 | 91022137 | | |
| 84 | 84146 | ZNF644 | exon_01_nc.3 | chr1: | chr1: | UGUACGCUACUCGGCUCCAG | + |
| | | | | 91022056- | 91022061- | | |
| | | | | 91022081 | 91022080 | | |
| 85 | 84146 | ZNF644 | exon_01_nc.3 | chr1: | chr1: | ACGCCGCUCUGUACGCUACU | + |
| | | | | 91022056- | 91022052- | | |
| | | | | 91022081 | 91022071 | | |
| 86 | 84146 | ZNF644 | exon_01_nc.3 | chr1: | chr1: | GAGCCGAGUAGCGUACAGAG | − |
| | | | | 91022056- | 91022058- | | |
| | | | | 91022081 | 91022077 | | |
| 87 | 84146 | ZNF644 | exon_01_nc.4 | chr1: | chr1: | UACAGAGCGGCGUGUGACGC | − |
| | | | | 91022036- | 91022045- | | |
| | | | | 91022055 | 91022064 | | |
| 88 | 84146 | ZNF644 | exon_01_nc.4 | chr1: | chr1: | ACAGAGCGGCGUGUGACGCG | − |
| | | | | 91022036- | 91022044- | | |
| | | | | 91022055 | 91022063 | | |
| 89 | 84146 | ZNF644 | exon_01_nc.5 | chr1: | chr1: | CGUCAAACCGGGGCGACGUU | + |
| | | | | 91022015- | 91022006- | | |
| | | | | 91022035 | 91022025 | | |
| 90 | 84146 | ZNF644 | exon_01_nc.5 | chr1: | chr1: | GCGUCAAACCGGGGCGACGU | + |
| | | | | 91022015- | 91022005- | | |
| | | | | 91022035 | 91022024 | | |
| 91 | 84146 | ZNF644 | exon_01_nc.5 | chr1: | chr1: | GCGUGCUCCCAACGUCGCCC | − |
| | | | | 91022015- | 91022016- | | |
| | | | | 91022035 | 91022035 | | |
| 92 | 84146 | ZNF644 | exon_01_nc.6 | chr1: | chr1: | GUGCCGUGUGCGUCAAACCG | + |
| | | | | 91021990- | 91021996- | | |
| | | | | 91022014 | 91022015 | | |
| 93 | 84146 | ZNF644 | exon_01_nc.6 | chr1: | chr1: | GGUGCCGUGUGCGUCAAACC | + |
| | | | | 91021990- | 91021995- | | |
| | | | | 91022014 | 91022014 | | |
| 94 | 84146 | ZNF644 | exon_01_nc.6 | chr1: | chr1: | UGGUGCCGUGUGCGUCAAAC | + |
| | | | | 91021990- | 91021994- | | |
| | | | | 91022014 | 91022013 | | |
| 95 | 84146 | ZNF644 | exon_01_nc.6 | chr1: | chr1: | UCGCCCCGGUUUGACGCACA | − |
| | | | | 91021990- | 91022002- | | |
| | | | | 91022014 | 91022021 | | |
| 96 | 84146 | ZNF644 | exon_01_nc.6 | chr1: | chr1: | AACCCCUGAAACUCACAGUU | + |
| | | | | 91021990- | 91021974- | | |
| | | | | 91022014 | 91021993 | | |
| 97 | 84146 | ZNF644 | intron_01 | chr1: | chr1: | GGCACCAAACUGUGAGUUUC | − |
| | | | | 91021726- | 91021981- | | |
| | | | | 91021989 | 91022000 | | |
| 98 | 84146 | ZNF644 | intron_01 | chr1: | chr1: | GGUUAUUUGGUCAGAUUCGC | − |
| | | | | 91021726- | 91021958- | | |
| | | | | 91021989 | 91021977 | | |
| 99 | 84146 | ZNF644 | intron_01 | chr1: | chr1: | GUUAUUUGGUCAGAUUCGCU | − |
| | | | | 91021726- | 91021957- | | |
| | | | | 91021989 | 91021976 | | |
| 100 | 84146 | ZNF644 | intron_01 | chr1: | chr1: | AGAUUCGCUGGGACCCGAGU | − |
| | | | | 91021726- | 91021946- | | |
| | | | | 91021989 | 91021965 | | |
| 101 | 84146 | ZNF644 | intron_01 | chr1: | chr1: | CCAGAGGCCCGAAACCCACU | + |
| | | | | 91021726- | 91021929- | | |
| | | | | 91021989 | 91021948 | | |
| 102 | 84146 | ZNF644 | intron_01 | chr1: | chr1: | CUGGGACCCGAGUGGGUUUC | − |
| | | | | 91021726- | 91021939- | | |
| | | | | 91021989 | 91021958 | | |
| 103 | 84146 | ZNF644 | intron_01 | chr1: | chr1: | CCGAGUGGGUUUCGGGCCUC | − |
| | | | | 91021726- | 91021932- | | |
| | | | | 91021989 | 91021951 | | |
| 104 | 84146 | ZNF644 | intron_01 | chr1: | chr1: | CGAGUGGGUUUCGGGCCUCU | − |
| | | | | 91021726- | 91021931- | | |
| | | | | 91021989 | 91021950 | | |
| 105 | 84146 | ZNF644 | intron_01 | chr1: | chr1: | GGUUUCGGGCCUCUGGGCUG | − |
| | | | | 91021726- | 91021925- | | |
| | | | | 91021989 | 91021944 | | |
| 106 | 84146 | ZNF644 | intron_01 | chr1: | chr1: | UGAGGGUGGCGGAAGAACUU | − |
| | | | | 91021726- | 91021890- | | |
| | | | | 91021989 | 91021909 | | |
| 107 | 84146 | ZNF644 | intron_01 | chr1: | chr1: | GAGGGUGGCGGAAGAACUUG | − |
| | | | | 91021726- | 91021889- | | |
| | | | | 91021989 | 91021908 | | |
| 108 | 84146 | ZNF644 | intron_01 | chr1: | chr1: | AACUUGGGGGACCCGGCCUC | − |
| | | | | 91021726- | 91021875- | | |
| | | | | 91021989 | 91021894 | | |
| 109 | 84146 | ZNF644 | intron_01 | chr1: | chr1: | AGCUCCCCGGGCCCGAGAGC | + |
| | | | | 91021726- | 91021853- | | |
| | | | | 91021989 | 91021872 | | |
| 110 | 84146 | ZNF644 | intron_01 | chr1: | chr1: | CGGGGAGCUACGCCCUAGGC | − |
| | | | | 91021726- | 91021842- | | |
| | | | | 91021989 | 91021861 | | |
| 111 | 84146 | ZNF644 | intron_01 | chr1: | chr1: | GGGGAGCUACGCCCUAGGCU | − |
| | | | | 91021726- | 91021841- | | |
| | | | | 91021989 | 91021860 | | |
| 112 | 84146 | ZNF644 | intron_01 | chr1: | chr1: | AUGCGUCCUUUUGGAUGGCU | + |
| | | | | 91021726- | 91021814- | | |
| | | | | 91021989 | 91021833 | | |
| 113 | 84146 | ZNF644 | intron_01 | chr1: | chr1: | AAUGCGUCCUUUUGGAUGGC | + |
| | | | | 91021726- | 91021813- | | |
| | | | | 91021989 | 91021832 | | |
| 114 | 84146 | ZNF644 | intron_01 | chr1: | chr1: | GCAAAAUGCGUCCUUUUGGA | + |
| | | | | 91021726- | 91021809- | | |
| | | | | 91021989 | 91021828 | | |
| 115 | 84146 | ZNF644 | intron_01 | chr1: | chr1: | UGCGGCAAAAUGCGUCCUUU | + |
| | | | | 91021726- | 91021805- | | |
| | | | | 91021989 | 91021824 | | |
| 116 | 84146 | ZNF644 | intron_01 | chr1: | chr1: | AAGGACGCAUUUUGCCGCAC | − |
| | | | | 91021726- | 91021804- | | |
| | | | | 91021989 | 91021823 | | |
| 117 | 84146 | ZNF644 | intron_01 | chr1: | chr1: | UUUUGCCGCACAGGCUAAGA | − |
| | | | | 91021726- | 91021795- | | |
| | | | | 91021989 | 91021814 | | |
| 118 | 84146 | ZNF644 | intron_01 | chr1: | chr1: | CGCACAGGCUAAGAUGGCGG | − |
| | | | | 91021726- | 91021789- | | |
| | | | | 91021989 | 91021808 | | |
| 119 | 84146 | ZNF644 | intron_01 | chr1: | chr1: | GGCGGAGUUGGUGCUACACA | − |
| | | | | 91021726- | 91021756- | | |
| | | | | 91021989 | 91021775 | | |
| 120 | 84146 | ZNF644 | intron_01 | chr1: | chr1: | UACACAAGGAGCCCGGGCCC | − |
| | | | | 91021726- | 91021742- | | |
| | | | | 91021989 | 91021761 | | |
| 121 | 84146 | ZNF644 | intron_01 | chr1: | chr1: | GCCUCCCCGAACCCGGGGCC | + |
| | | | | 91021726- | 91021727- | | |
| | | | | 91021989 | 91021746 | | |
| 122 | 84146 | ZNF644 | intron_01 | chr1: | chr1: | AGGAGCCCGGGCCCCGGGUU | − |
| | | | | 91021726- | 91021736- | | |
| | | | | 91021989 | 91021755 | | |
| 123 | 84146 | ZNF644 | intron_01 | chr1: | chr1: | GAGCCCGGGCCCCGGGUUCG | − |
| | | | | 91021726- | 91021734- | | |
| | | | | 91021989 | 91021753 | | |
| 124 | 84146 | ZNF644 | exon_02_nc | chr1: | chr1: | GUGGCCCCCAGCGACCGCCG | + |
| | | | | 91021689- | 91021699- | | |
| | | | | 91021725 | 91021718 | | |
| 125 | 84146 | ZNF644 | exon_02_nc | chr1: | chr1: | GCGGCGGUCGCUGGGGGCCA | − |
| | | | | 91021689- | 91021700- | | |
| | | | | 91021725 | 91021719 | | |
| 126 | 84146 | ZNF644 | exon_02_nc | chr1: | chr1: | GCCGCUCACCUCAGGCUCCG | + |
| | | | | 91021689- | 91021680- | | |
| | | | | 91021725 | 91021699 | | |
| 127 | 84146 | ZNF644 | intron_02 | chr1: | chr1: | CGGCGGCCGCCGCUCACCUC | + |
| | | | | 91021509- | 91021672- | | |
| | | | | 91021688 | 91021691 | | |
| 128 | 84146 | ZNF644 | intron_02 | chr1: | chr1: | ACGGAGCCUGAGGUGAGCGG | − |
| | | | | 91021509- | 91021681- | | |
| | | | | 91021688 | 91021700 | | |
| 129 | 84146 | ZNF644 | intron_02 | chr1: | chr1: | ACUCGGCGGGGGGAUGGGGG | − |
| | | | | 91021509- | 91021640- | | |
| | | | | 91021688 | 91021659 | | |
| 130 | 84146 | ZNF644 | intron_02 | chr1: | chr1: | CGGAUCCUCCGACGCCGCAG | + |
| | | | | 91021509- | 91021608- | | |
| | | | | 91021688 | 91021627 | | |
| 131 | 84146 | ZNF644 | intron_02 | chr1: | chr1: | GGGGCUUUCCGCUGCGGCGU | − |
| | | | | 91021509- | 91021619- | | |
| | | | | 91021688 | 91021638 | | |
| 132 | 84146 | ZNF644 | intron_02 | chr1: | chr1: | GCUUUCCGCUGCGGCGUCGG | − |
| | | | | 91021509- | 91021616- | | |
| | | | | 91021688 | 91021635 | | |
| 133 | 84146 | ZNF644 | intron_02 | chr1: | chr1: | GGCGUCGGAGGAUCCGGUAG | |
| | | | | 91021509- | 91021604- | | |
| | | | | 91021688 | 91021623 | | |
| 134 | 84146 | ZNF644 | intron_02 | chr1: | chr1: | AGCCCCUUUGUGUCCGCUAC | + |
| | | | | 91021509- | 91021588- | | |
| | | | | 91021688 | 91021607 | | |
| 135 | 84146 | ZNF644 | intron_02 | chr1: | chr1: | GGAUCCGGUAGCGGACACAA | − |
| | | | | 91021509- | 91021595- | | |
| | | | | 91021688 | 91021614 | | |
| 136 | 84146 | ZNF644 | intron_02 | chr1: | chr1: | GAUCCGGUAGCGGACACAAA | − |
| | | | | 91021509- | 91021594- | | |
| | | | | 91021688 | 91021613 | | |
| 137 | 84146 | ZNF644 | intron_02 | chr1: | chr1: | AUCCGGUAGCGGACACAAAG | − |
| | | | | 91021509- | 91021593- | | |
| | | | | 91021688 | 91021612 | | |
| 138 | 84146 | ZNF644 | intron_02 | chr1: | chr1: | CGGACACAAAGGGGCUGAGC | − |
| | | | | 91021509- | 91021584- | | |
| | | | | 91021688 | 91021603 | | |
| 139 | 84146 | ZNF644 | intron_02 | chr1: | chr1: | CUGAGCUGGGCACUAGACUU | − |
| | | | | 91021509- | 91021570- | | |
| | | | | 91021688 | 91021589 | | |
| 140 | 84146 | ZNF644 | intron_02 | chr1: | chr1: | UGAGCUGGGCACUAGACUUG | − |
| | | | | 91021509- | 91021569- | | |
| | | | | 91021688 | 91021588 | | |
| 141 | 84146 | ZNF644 | intron_02 | chr1: | chr1: | GAGCUGGGCACUAGACUUGG | − |
| | | | | 91021509- | 91021568- | | |
| | | | | 91021688 | 91021587 | | |
| 142 | 84146 | ZNF644 | intron_02 | chr1: | chr1: | CGAGCAGCCAGUGAGCAGUA | − |
| | | | | 91021509- | 91021524- | | |
| | | | | 91021688 | 91021543 | | |
| 143 | 84146 | ZNF644 | exon_03_nc.1 | chr1: | chr1: | CUCCCCAAGCCGCCUGAUCU | + |
| | | | | 91021474- | 91021490- | | |
| | | | | 91021508 | 91021509 | | |
| 144 | 84146 | ZNF644 | exon_03_nc.1 | chr1: | chr1: | GGGGCCGAGAUCAGGCGGCU | − |
| | | | | 91021474- | 91021497- | | |
| | | | | 91021508 | 91021516 | | |
| 145 | 84146 | ZNF644 | exon_03_nc.1 | chr1: | chr1: | GGGCCGAGAUCAGGCGGCUU | − |
| | | | | 91021474- | 91021496- | | |
| | | | | 91021508 | 91021515 | | |
| 146 | 84146 | ZNF644 | exon_03_nc.1 | chr1: | chr1: | GGCCGAGAUCAGGCGGCUUG | − |
| | | | | 91021474- | 91021495- | | |
| | | | | 91021508 | 91021514 | | |
| 147 | 84146 | ZNF644 | exon_03_nc.1 | chr1: | chr1: | GCAUUGCAAUGGCGACCGCC | + |
| | | | | 91021474- | 91021467- | | |
| | | | | 91021508 | 91021486 | | |
| 148 | 84146 | ZNF644 | exon_03_nc.2 | chr1: | chr1: | CGUUACUCAGUGCAUUGCAA | + |
| | | | | 91021458- | 91021456- | | |
| | | | | 91021473 | 91021475 | | |
| 149 | 84146 | ZNF644 | exon_03_nc.2 | chr1: | chr1: | UUGCAAUGCACUGAGUAACG | − |
| | | | | 91021458- | 91021456- | | |
| | | | | 91021473 | 91021475 | | |
| 150 | 84146 | ZNF644 | exon_03_nc.3 | chr1: | chr1: | CAAUGCACUGAGUAACGGGG | − |
| | | | | 91021273- | 91021453- | | |
| | | | | 91021457 | 91021472 | | |
| 151 | 84146 | ZNF644 | exon_03_nc.3 | chr1: | chr1: | AAUGCACUGAGUAACGGGGC | − |
| | | | | 91021273- | 91021452- | | |
| | | | | 91021457 | 91021471 | | |
| 152 | 84146 | ZNF644 | exon_03_nc.3 | chr1: | chr1: | GCAGCCUCAACGAGAGGGGU | + |
| | | | | 91021273- | 91021413- | | |
| | | | | 91021457 | 91021432 | | |
| 153 | 84146 | ZNF644 | exon_03_nc.3 | chr1: | chr1: | CCGAGCAGCCUCAACGAGAG | + |
| | | | | 91021273- | 91021409- | | |
| | | | | 91021457 | 91021428 | | |
| 154 | 84146 | ZNF644 | exon_03_nc.3 | chr1: | chr1: | ACCGAGCAGCCUCAACGAGA | + |
| | | | | 91021273- | 91021408- | | |
| | | | | 91021457 | 91021427 | | |
| 155 | 84146 | ZNF644 | exon_03_nc.3 | chr1: | chr1: | AACCGAGCAGCCUCAACGAG | + |
| | | | | 91021273- | 91021407- | | |
| | | | | 91021457 | 91021426 | | |
| 156 | 84146 | ZNF644 | exon_03_nc.3 | chr1: | chr1: | GUCACCAACCCCUCUCGUUG | − |
| | | | | 91021273- | 91021420- | | |
| | | | | 91021457 | 91021439 | | |
| 157 | 84146 | ZNF644 | exon_03_nc.3 | chr1: | chr1: | CCCCUCUCGUUGAGGCUGCU | − |
| | | | | 91021273- | 91021412- | | |
| | | | | 91021457 | 91021431 | | |
| 158 | 84146 | ZNF644 | exon_03_nc.3 | chr1: | chr1: | UCGUUGAGGCUGCUCGGUUG | − |
| | | | | 91021273- | 91021406- | | |
| | | | | 91021457 | 91021425 | | |
| 159 | 84146 | ZNF644 | exon_03_nc.3 | chr1: | chr1: | UCGGUUGUGGCCAGAAACGC | − |
| | | | | 91021273- | 91021393- | | |
| | | | | 91021457 | 91021412 | | |
| 160 | 84146 | ZNF644 | exon_03_nc.3 | chr1: | chr1: | CUACACCCCUCCCCUUACUU | − |
| | | | | 91021273- | 91021354- | | |
| | | | | 91021457 | 91021373 | | |
| 161 | 84146 | ZNF644 | exon_03_nc.3 | chr1: | chr1: | UGCGGUCCUGUCCCAAGUAA | + |
| | | | | 91021273- | 91021340- | | |
| | | | | 91021457 | 91021359 | | |
| 162 | 84146 | ZNF644 | exon_03_nc.3 | chr1: | chr1: | AUGCGGUCCUGUCCCAAGUA | + |
| | | | | 91021273- | 91021339- | | |
| | | | | 91021457 | 91021358 | | |
| 163 | 84146 | ZNF644 | exon_03_nc.3 | chr1: | chr1: | AUCUUGCAGGACGGGGGGAG | + |
| | | | | 91021273- | 91021310- | | |
| | | | | 91021457 | 91021329 | | |
| 164 | 84146 | ZNF644 | exon_03_nc.3 | chr1: | chr1: | GAUCUUGCAGGACGGGGGGA | + |
| | | | | 91021273- | 91021309- | | |
| | | | | 91021457 | 91021328 | | |
| 165 | 84146 | ZNF644 | exon_03_nc.3 | chr1: | chr1: | AUAAGAUCUUGCAGGACGGG | + |
| | | | | 91021273- | 91021305- | | |
| | | | | 91021457 | 91021324 | | |
| 166 | 84146 | ZNF644 | exon_03_nc.3 | chr1: | chr1: | CAUAAGAUCUUGCAGGACGG | + |
| | | | | 91021273- | 91021304- | | |
| | | | | 91021457 | 91021323 | | |
| 167 | 84146 | ZNF644 | exon_03_nc.3 | chr1: | chr1: | GCAUAAGAUCUUGCAGGACG | + |
| | | | | 91021273- | 91021303- | | |
| | | | | 91021457 | 91021322 | | |
| 168 | 84146 | ZNF644 | exon_03_nc.3 | chr1: | chr1: | CGCAUAAGAUCUUGCAGGAC | + |
| | | | | 91021273- | 91021302- | | |
| | | | | 91021457 | 91021321 | | |
| 169 | 84146 | ZNF644 | exon_03_nc.3 | chr1: | chr1: | CCGCAUAAGAUCUUGCAGGA | + |
| | | | | 91021273- | 91021301- | | |
| | | | | 91021457 | 91021320 | | |
| 170 | 84146 | ZNF644 | exon_03_nc.3 | chr1: | chr1: | CUAGCCGCAUAAGAUCUUGC | + |
| | | | | 91021273- | 91021297- | | |
| | | | | 91021457 | 91021316 | | |
| 171 | 84146 | ZNF644 | exon_03_nc.3 | chr1: | chr1: | CCGUCCUGCAAGAUCUUAUG | − |
| | | | | 91021273- | 91021304- | | |
| | | | | 91021457 | 91021323 | | |
| 172 | 84146 | ZNF644 | exon_03_nc.3 | chr1: | chr1: | CUGCAAGAUCUUAUGCGGCU | − |
| | | | | 91021273- | 91021299- | | |
| | | | | 91021457 | 91021318 | | |
| 173 | 84146 | ZNF644 | exon_03_nc.3 | chr1: | chr1: | AUCUUAUGCGGCUAGGUGCC | − |
| | | | | 91021273- | 91021292- | | |
| | | | | 91021457 | 91021311 | | |
| 174 | 84146 | ZNF644 | exon_03_nc.3 | chr1: | chr1: | UCUUAUGCGGCUAGGUGCCU | − |
| | | | | 91021273- | 91021291- | | |
| | | | | 91021457 | 91021310 | | |
| 175 | 84146 | ZNF644 | exon_03_nc.3 | chr1: | chr1: | ACCUCUUGCUGUUACUUCCC | + |
| | | | | 91021273- | 91021271- | | |
| | | | | 91021457 | 91021290 | | |
| 176 | 84146 | ZNF644 | intron_03 | chr1: | chr1: | GGUAAAUCGCUCGAAGUGUU | − |
| | | | | 91021202- | 91021254- | | |
| | | | | 91021272 | 91021273 | | |
| 177 | 84146 | ZNF644 | intron_03 | chr1: | chr1: | GUAAAUCGCUCGAAGUGUUU | − |
| | | | | 91021202- | 91021253- | | |
| | | | | 91021272 | 91021272 | | |
| 178 | 84146 | ZNF644 | intron_03 | chr1: | chr1: | CUCGAAGUGUUUGGGUUGCC | − |
| | | | | 91021202- | 91021245- | | |
| | | | | 91021272 | 91021264 | | |
| 179 | 84146 | ZNF644 | intron_03 | chr1: | chr1: | UCGAAGUGUUUGGGUUGCCA | − |
| | | | | 91021202- | 91021244- | | |
| | | | | 91021272 | 91021263 | | |
| 180 | 84146 | ZNF644 | intron_03 | chr1: | chr1: | CGAAGUGUUUGGGUUGCCAG | − |
| | | | | 91021202- | 91021243- | | |
| | | | | 91021272 | 91021262 | | |
| 181 | 84146 | ZNF644 | intron_03 | chr1: | chr1: | GAGAGGCAAAAAGCUUUAGC | + |
| | | | | 91021202- | 91021205- | | |
| | | | | 91021272 | 91021224 | | |
| 182 | 84146 | ZNF644 | exon_04_nc.1 | chr1: | chr1: | GUAACUUCCCAGGGACACAA | + |
| | | | | 91020529- | 91021165- | | |
| | | | | 91021201 | 91021184 | | |
| 183 | 84146 | ZNF644 | exon_04_nc.1 | chr1: | chr1: | UCUGGCAGAGUAACUUCCCA | + |
| | | | | 91020529- | 91021156- | | |
| | | | | 91021201 | 91021175 | | |
| 184 | 84146 | ZNF644 | exon_04_nc.1 | chr1: | chr1: | CUCUGGCAGAGUAACUUCCC | + |
| | | | | 91020529- | 91021155- | | |
| | | | | 91021201 | 91021174 | | |
| 185 | 84146 | ZNF644 | exon_04_nc.1 | chr1: | chr1: | CUUCAGGUUCACGGGAGAAG | + |
| | | | | 91020529- | 91021117- | | |
| | | | | 91021201 | 91021136 | | |
| 186 | 84146 | ZNF644 | exon_04_nc.1 | chr1: | chr1: | ACAACUCAUCUUCAGGUUCA | + |
| | | | | 91020529- | 91021108- | | |
| | | | | 91021201 | 91021127 | | |
| 187 | 84146 | ZNF644 | exon_04_nc.1 | chr1: | chr1: | GUCUGGGACAACUCAUCUUC | + |
| | | | | 91020529- | 91021101- | | |
| | | | | 91021201 | 91021120 | | |
| 188 | 84146 | ZNF644 | exon_04_nc.1 | chr1: | chr1: | CCAAUCACUGAGAAGAGUCU | + |
| | | | | 91020529- | 91021085- | | |
| | | | | 91021201 | 91021104 | | |
| 189 | 84146 | ZNF644 | exon_04_nc.1 | chr1: | chr1: | UCCAAUCACUGAGAAGAGUC | + |
| | | | | 91020529- | 91021084- | | |
| | | | | 91021201 | 91021103 | | |
| 190 | 84146 | ZNF644 | exon_04_nc.1 | chr1: | chr1: | CCCAGACUCUUCUCAGUGAU | − |
| | | | | 91020529- | 91021088- | | |
| | | | | 91021201 | 91021107 | | |
| 191 | 84146 | ZNF644 | exon_04_nc.1 | chr1: | chr1: | AACUUAGGUGGUGGAGCUAA | − |
| | | | | 91020529- | 91021064- | | |
| | | | | 91021201 | 91021083 | | |
| 192 | 84146 | ZNF644 | exon_04_nc.1 | chr1: | chr1: | CUCUUGUUGGGAUCCAUCUA | − |
| | | | | 91020529- | 91021013- | | |
| | | | | 91021201 | 91021032 | | |
| 193 | 84146 | ZNF644 | exon_04_nc.1 | chr1: | chr1: | UAAUGUUGCUAAACCAUAGA | + |
| | | | | 91020529- | 91020997- | | |
| | | | | 91021201 | 91021016 | | |
| 194 | 84146 | ZNF644 | exon_04_nc.1 | chr1: | chr1: | CUAUGGUUUAGCAACAUUAG | − |
| | | | | 91020529- | 91020996- | | |
| | | | | 91021201 | 91021015 | | |
| 195 | 84146 | ZNF644 | exon_04_nc.1 | chr1: | chr1: | UGUGAUCAUCAAACUCAACG | − |
| | | | | 91020529- | 91020911- | | |
| | | | | 91021201 | 91020930 | | |
| 196 | 84146 | ZNF644 | exon_04_nc.1 | chr1: | chr1: | GUGAUCAUCAAACUCAACGA | − |
| | | | | 91020529- | 91020910- | | |
| | | | | 91021201 | 91020929 | | |
| 197 | 84146 | ZNF644 | exon_04_nc.1 | chr1: | chr1: | AACGAGGGGAUAAACAUAAU | − |
| | | | | 91020529- | 91020895- | | |
| | | | | 91021201 | 91020914 | | |
| 198 | 84146 | ZNF644 | exon_04_nc.1 | chr1: | chr1: | ACGAGGGGAUAAACAUAAUU | − |
| | | | | 91020529- | 91020894- | | |
| | | | | 91021201 | 91020913 | | |
| 199 | 84146 | ZNF644 | exon_04_nc.1 | chr1: | chr1: | CGAGGGGAUAAACAUAAUUG | − |
| | | | | 91020529- | 91020893- | | |
| | | | | 91021201 | 91020912 | | |
| 200 | 84146 | ZNF644 | exon_04_nc.1 | chr1: | chr1: | GAGGGGAUAAACAUAAUUGG | − |
| | | | | 91020529- | 91020892- | | |
| | | | | 91021201 | 91020911 | | |
| 201 | 84146 | ZNF644 | exon_04_nc.1 | chr1: | chr1: | ACAUAAUUGGGGGCGAAUGA | − |
| | | | | 91020529- | 91020882- | | |
| | | | | 91021201 | 91020901 | | |
| 202 | 84146 | ZNF644 | exon_04_nc.1 | chr1: | chr1: | GGACUGGACAGGAUAGUCGG | + |
| | | | | 91020529- | 91020817- | | |
| | | | | 91021201 | 91020836 | | |
| 203 | 84146 | ZNF644 | exon_04_nc.1 | chr1: | chr1: | GCGUGGAAUUAGGACUGGAC | + |
| | | | | 91020529- | 91020806- | | |
| | | | | 91021201 | 91020825 | | |
| 204 | 84146 | ZNF644 | exon_04_nc.1 | chr1: | chr1: | AACAAGCGUGGAAUUAGGAC | + |
| | | | | 91020529- | 91020801- | | |
| | | | | 91021201 | 91020820 | | |
| 205 | 84146 | ZNF644 | exon_04_nc.1 | chr1: | chr1: | GAUGCAACAAGCGUGGAAUU | + |
| | | | | 91020529- | 91020796- | | |
| | | | | 91021201 | 91020815 | | |
| 206 | 84146 | ZNF644 | exon_04_nc.1 | chr1: | chr1: | CCAUCCAGAUGCAACAAGCG | + |
| | | | | 91020529- | 91020789- | | |
| | | | | 91021201 | 91020808 | | |
| 207 | 84146 | ZNF644 | exon_04_nc.1 | chr1: | chr1: | AAUUCCACGCUUGUUGCAUC | − |
| | | | | 91020529- | 91020796- | | |
| | | | | 91021201 | 91020815 | | |
| 208 | 84146 | ZNF644 | exon_04_nc.1 | chr1: | chr1: | CCACGCUUGUUGCAUCUGGA | − |
| | | | | 91020529- | 91020792- | | |
| | | | | 91021201 | 91020811 | | |
| 209 | 84146 | ZNF644 | exon_04_nc.1 | chr1: | chr1: | UAAAGCAAACUACUGCAGGG | + |
| | | | | 91020529- | 91020742- | | |
| | | | | 91021201 | 91020761 | | |
| 210 | 84146 | ZNF644 | exon_04_nc.1 | chr1: | chr1: | CAACUAAAGCAAACUACUGC | + |
| | | | | 91020529- | 91020738- | | |
| | | | | 91021201 | 91020757 | | |
| 211 | 84146 | ZNF644 | exon_04_nc.1 | chr1: | chr1: | CAUUUUUUGGUCCCAUGAGA | − |
| | | | | 91020529- | 91020714- | | |
| | | | | 91021201 | 91020733 | | |
| 212 | 84146 | ZNF644 | exon_04_nc.1 | chr1: | chr1: | AGUUGCAUGUGUAAGUAUCU | − |
| | | | | 91020529- | 91020627- | | |
| | | | | 91021201 | 91020646 | | |
| 213 | 84146 | ZNF644 | exon_04_nc.1 | chr1: | chr1: | UAAGUAUCUAGGGAACUUGA | − |
| | | | | 91020529- | 91020616- | | |
| | | | | 91021201 | 91020635 | | |
| 214 | 84146 | ZNF644 | exon_04_nc.1 | chr1: | chr1: | CAGUAAUAUUGCAUUUUGGU | − |
| | | | | 91020529- | 91020593- | | |
| | | | | 91021201 | 91020612 | | |
| 215 | 84146 | ZNF644 | exon_04_nc.1 | chr1: | chr1: | AAAACCUCGUUUUUAUAAUA | − |
| | | | | 91020529- | 91020560- | | |
| | | | | 91021201 | 91020579 | | |
| 216 | 84146 | ZNF644 | exon_04_nc.1 | chr1: | chr1: | UCUAGCUUAGACAUUUUAAU | − |
| | | | | 91020529- | 91020531- | | |
| | | | | 91021201 | 91020550 | | |
| 217 | 84146 | ZNF644 | exon_04_nc.2 | chr1: | chr1: | CUUGAUUAGUAUUAAUGCAG | − |
| | | | | 91020448- | 91020496- | | |
| | | | | 91020528 | 91020515 | | |
| 218 | 84146 | ZNF644 | intron_04 | chr1: | chr1: | AAACACCUAAUGUUUUGUGU | − |
| | | | | 90982371- | 91020370- | | |
| | | | | 91020447 | 91020389 | | |
| 219 | 84146 | ZNF644 | intron_04 | chr1: | chr1: | UGGCAAUUAACAGAAUUCCU | − |
| | | | | 90982371- | 91020350- | | |
| | | | | 91020447 | 91020369 | | |
| 220 | 84146 | ZNF644 | intron_04 | chr1: | chr1: | ACAGUCUUGUAUUAAUGAAU | − |
| | | | | 90982371- | 91020185- | | |
| | | | | 91020447 | 91020204 | | |
| 221 | 84146 | ZNF644 | intron_04 | chr1: | chr1: | ACUGUAAAACUUUAUGAUCC | + |
| | | | | 90982371- | 91020132- | | |
| | | | | 91020447 | 91020151 | | |
| 222 | 84146 | ZNF644 | intron_04 | chr1: | chr1: | CAAUAUAUUGUAAAAGAGGA | + |
| | | | | 90982371- | 91020079- | | |
| | | | | 91020447 | 91020098 | | |
| 223 | 84146 | ZNF644 | intron_04 | chr1: | chr1: | ACCAGAGAUGAUUAAGUUAG | − |
| | | | | 90982371- | 91020056- | | |
| | | | | 91020447 | 91020075 | | |
| 224 | 84146 | ZNF644 | intron_04 | chr1: | chr1: | UGAUUAAGUUAGUGGCAUAA | − |
| | | | | 90982371- | 91020048- | | |
| | | | | 91020447 | 91020067 | | |
| 225 | 84146 | ZNF644 | intron_04 | chr1: | chr1: | AUACAAACAGCAAUGGGUAG | + |
| | | | | 90982371- | 91020007- | | |
| | | | | 91020447 | 91020026 | | |
| 226 | 84146 | ZNF644 | intron_04 | chr1: | chr1: | UGACAAAUACAAACAGCAAU | + |
| | | | | 90982371- | 91020001- | | |
| | | | | 91020447 | 91020020 | | |
| 227 | 84146 | ZNF644 | intron_04 | chr1: | chr1: | GUUUGUAUUUGUCAUAUUGC | |
| | | | | 90982371- | 91019995- | | |
| | | | | 91020447 | 91020014 | | |
| 228 | 84146 | ZNF644 | intron_04 | chr1: | chr1: | UAAGUUAAAAACUUUCAUAU | − |
| | | | | 90982371- | 91019866- | | |
| | | | | 91020447 | 91019885 | | |
| 229 | 84146 | ZNF644 | intron_04 | chr1: | chr1: | UUCUGGGUCAAGACUGCAUU | + |
| | | | | 90982371- | 91019427- | | |
| | | | | 91020447 | 91019446 | | |
| 230 | 84146 | ZNF644 | intron_04 | chr1: | chr1: | GUUAUUGCCAGUUAUUAUGU | − |
| | | | | 90982371- | 91019341- | | |
| | | | | 91020447 | 91019360 | | |
| 231 | 84146 | ZNF644 | intron_04 | chr1: | chr1: | AGACUACUACUUCUACUGAG | − |
| | | | | 90982371- | 91019203- | | |
| | | | | 91020447 | 91019222 | | |
| 232 | 84146 | ZNF644 | intron_04 | chr1: | chr1: | UCUACUGAGUGGUACCUCUA | − |
| | | | | 90982371- | 91019192- | | |
| | | | | 91020447 | 91019211 | | |
| 233 | 84146 | ZNF644 | intron_04 | chr1: | chr1: | UGGUACCUCUAAGGUCUGUU | − |
| | | | | 90982371- | 91019183- | | |
| | | | | 91020447 | 91019202 | | |
| 234 | 84146 | ZNF644 | intron_04 | chr1: | chr1: | CAAGUAGUGACAGUCAUUUA | − |
| | | | | 90982371- | 91019152- | | |
| | | | | 91020447 | 91019171 | | |
| 235 | 84146 | ZNF644 | intron_04 | chr1: | chr1: | GUAGUGACAGUCAUUUAUGG | − |
| | | | | 90982371- | 91019149- | | |
| | | | | 91020447 | 91019168 | | |
| 236 | 84146 | ZNF644 | intron_04 | chr1: | chr1: | UGAAAAUUAGAGUCACUUUC | − |
| | | | | 90982371- | 91018943- | | |
| | | | | 91020447 | 91018962 | | |
| 237 | 84146 | ZNF644 | intron_04 | chr1: | chr1: | GGAAAAUGUUUAAGGUUGGC | + |
| | | | | 90982371- | 91018889- | | |
| | | | | 91020447 | 91018908 | | |
| 238 | 84146 | ZNF644 | intron_04 | chr1: | chr1: | AACUGGAAAAUGUUUAAGGU | + |
| | | | | 90982371- | 91018885- | | |
| | | | | 91020447 | 91018904 | | |
| 239 | 84146 | ZNF644 | intron_04 | chr1: | chr1: | CUUGAAUGACAAACUAAAAC | + |
| | | | | 90982371- | 91018868- | | |
| | | | | 91020447 | 91018887 | | |
| 240 | 84146 | ZNF644 | intron_04 | chr1: | chr1: | UGAGACUGUUAAUGGGUAGC | − |
| | | | | 90982371- | 91018825- | | |
| | | | | 91020447 | 91018844 | | |
| 241 | 84146 | ZNF644 | intron_04 | chr1: | chr1: | UUCAAAGAGAAUAGAAGUGG | − |
| | | | | 90982371- | 91018787- | | |
| | | | | 91020447 | 91018806 | | |
| 242 | 84146 | ZNF644 | intron_04 | chr1: | chr1: | GGAAAAGUGAAUCUUAUUCU | |
| | | | | 90982371- | 91018656- | | |
| | | | | 91020447 | 91018675 | | |
| 243 | 84146 | ZNF644 | intron_04 | chr1: | chr1: | CAAUUGUAGCUUGCAGCAAA | − |
| | | | | 90982371- | 91018607- | | |
| | | | | 91020447 | 91018626 | | |
| 244 | 84146 | ZNF644 | intron_04 | chr1: | chr1: | GAGUGGAUACUGUAAUACUA | − |
| | | | | 90982371- | 91018465- | | |
| | | | | 91020447 | 91018484 | | |
| 245 | 84146 | ZNF644 | intron_04 | chr1: | chr1: | AGAAGUGGUGUUUUUGAUUC | − |
| | | | | 90982371- | 91018313- | | |
| | | | | 91020447 | 91018332 | | |
| 246 | 84146 | ZNF644 | intron_04 | chr1: | chr1: | UUUUGAUUCAGGUUUAUUAG | − |
| | | | | 90982371- | 91018302- | | |
| | | | | 91020447 | 91018321 | | |
| 247 | 84146 | ZNF644 | intron_04 | chr1: | chr1: | UUUGAUUCAGGUUUAUUAGA | − |
| | | | | 90982371- | 91018301- | | |
| | | | | 91020447 | 91018320 | | |
| 248 | 84146 | ZNF644 | intron_04 | chr1: | chr1: | UCAAAUCUCCCAAAACUCAA | + |
| | | | | 90982371- | 91018237- | | |
| | | | | 91020447 | 91018256 | | |
| 249 | 84146 | ZNF644 | intron_04 | chr1: | chr1: | UUUGGGAGAUUUGAGAAUCA | − |
| | | | | 90982371- | 91018231- | | |
| | | | | 91020447 | 91018250 | | |
| 250 | 84146 | ZNF644 | intron_04 | chr1: | chr1: | CUAGCACUAGCUCACCCUGC | − |
| | | | | 90982371- | 91018200- | | |
| | | | | 91020447 | 91018219 | | |
| 251 | 84146 | ZNF644 | intron_04 | chr1: | chr1: | AAUCUUUUAGCCAACCAGCA | + |
| | | | | 90982371- | 91018183- | | |
| | | | | 91020447 | 91018202 | | |
| 252 | 84146 | ZNF644 | intron_04 | chr1: | chr1: | AUCUUCAUGUUUGUUUCUUA | + |
| | | | | 90982371- | 91018149- | | |
| | | | | 91020447 | 91018168 | | |
| 253 | 84146 | ZNF644 | intron_04 | chr1: | chr1: | UUAGAGUCUUACUACCCUCU | + |
| | | | | 90982371- | 91018117- | | |
| | | | | 91020447 | 91018136 | | |
| 254 | 84146 | ZNF644 | intron_04 | chr1: | chr1: | UUUAGAGUCUUACUACCCUC | + |
| | | | | 90982371- | 91018116- | | |
| | | | | 91020447 | 91018135 | | |
| 255 | 84146 | ZNF644 | intron_04 | chr1: | chr1: | AGUAACUCCAUGGUUCAACU | + |
| | | | | 90982371- | 91017991- | | |
| | | | | 91020447 | 91018010 | | |
| 256 | 84146 | ZNF644 | intron_04 | chr1: | chr1: | AAAGGAUAGUAACUACUGCC | + |
| | | | | 90982371- | 91017664- | | |
| | | | | 91020447 | 91017683 | | |
| 257 | 84146 | ZNF644 | intron_04 | chr1: | chr1: | ACUAUCCUUUUUGAACUUCC | − |
| | | | | 90982371- | 91017654- | | |
| | | | | 91020447 | 91017673 | | |
| 258 | 84146 | ZNF644 | intron_04 | chr1: | chr1: | UGGGAACUCUUAUGUGAUUA | − |
| | | | | 90982371- | 91017634- | | |
| | | | | 91020447 | 91017653 | | |
| 259 | 84146 | ZNF644 | intron_04 | chr1: | chr1: | GCUGCUACUUCUGCCCUGGC | + |
| | | | | 90982371- | 91017465- | | |
| | | | | 91020447 | 91017484 | | |
| 260 | 84146 | ZNF644 | intron_04 | chr1: | chr1: | AGUGCACACUUCUCUUGGUU | − |
| | | | | 90982371- | 91017445- | | |
| | | | | 91020447 | 91017464 | | |
| 261 | 84146 | ZNF644 | intron_04 | chr1: | chr1: | UGGAUUAAGAUUAGCGUUUC | − |
| | | | | 90982371- | 91017410- | | |
| | | | | 91020447 | 91017429 | | |
| 262 | 84146 | ZNF644 | intron_04 | chr1: | chr1: | GGAUUAAGAUUAGCGUUUCU | − |
| | | | | 90982371- | 91017409- | | |
| | | | | 91020447 | 91017428 | | |
| 263 | 84146 | ZNF644 | intron_04 | chr1: | chr1: | AGGGCCAAGAUUAGUAGGAG | + |
| | | | | 90982371- | 91017370- | | |
| | | | | 91020447 | 91017389 | | |
| 264 | 84146 | ZNF644 | intron_04 | chr1: | chr1: | GAGGUAGGGCCAAGAUUAGU | + |
| | | | | 90982371- | 91017365- | | |
| | | | | 91020447 | 91017384 | | |
| 265 | 84146 | ZNF644 | intron_04 | chr1: | chr1: | AGCUAGCAGACAGAAGAGGU | + |
| | | | | 90982371- | 91017350- | | |
| | | | | 91020447 | 91017369 | | |
| 266 | 84146 | ZNF644 | intron_04 | chr1: | chr1: | CCACAGCUAGCAGACAGAAG | + |
| | | | | 90982371- | 91017346- | | |
| | | | | 91020447 | 91017365 | | |
| 267 | 84146 | ZNF644 | intron_04 | chr1: | chr1: | UGUGGUCUUCCCAUUUCAUU | − |
| | | | | 90982371- | 91017331- | | |
| | | | | 91020447 | 91017350 | | |
| 268 | 84146 | ZNF644 | intron_04 | chr1: | chr1: | AUUUUGGUAGAAUUGAAGCU | − |
| | | | | 90982371- | 91017230- | | |
| | | | | 91020447 | 91017249 | | |
| 269 | 84146 | ZNF644 | intron_04 | chr1: | chr1: | AAGCUGGGUUAGAGUGAUGA | − |
| | | | | 90982371- | 91017215- | | |
| | | | | 91020447 | 91017234 | | |
| 270 | 84146 | ZNF644 | intron_04 | chr1: | chr1: | UUAAUUUUCAGUUGUCUAUU | + |
| | | | | 90982371- | 91016777- | | |
| | | | | 91020447 | 91016796 | | |
| 271 | 84146 | ZNF644 | intron_04 | chr1: | chr1: | CCCCUCUCCUCCCCAAUAAA | + |
| | | | | 90982371- | 91016230- | | |
| | | | | 91020447 | 91016249 | | |
| 272 | 84146 | ZNF644 | intron_04 | chr1: | chr1: | CACCAUUUAUUGGGGAGGAG | − |
| | | | | 90982371- | 91016235- | | |
| | | | | 91020447 | 91016254 | | |
| 273 | 84146 | ZNF644 | intron_04 | chr1: | chr1: | ACCAUUUAUUGGGGAGGAGA | − |
| | | | | 90982371- | 91016234- | | |
| | | | | 91020447 | 91016253 | | |
| 274 | 84146 | ZNF644 | intron_04 | chr1: | chr1: | UUAUUGUUGGUUUGUGAAGG | − |
| | | | | 90982371- | 91016199- | | |
| | | | | 91020447 | 91016218 | | |
| 275 | 84146 | ZNF644 | intron_04 | chr1: | chr1: | UAUUGUUGGUUUGUGAAGGU | − |
| | | | | 90982371- | 91016198- | | |
| | | | | 91020447 | 91016217 | | |
| 276 | 84146 | ZNF644 | intron_04 | chr1: | chr1: | GUAAUUAUGCAUAUAUAAAG | − |
| | | | | 90982371- | 91016072- | | |
| | | | | 91020447 | 91016091 | | |
| 277 | 84146 | ZNF644 | intron_04 | chr1: | chr1: | UUAUGCAUAUAUAAAGAGGC | − |
| | | | | 90982371- | 91016068- | | |
| | | | | 91020447 | 91016087 | | |
| 278 | 84146 | ZNF644 | intron_04 | chr1: | chr1: | AGCCUUGAAGUAAAAUUGAC | − |
| | | | | 90982371- | 91016022- | | |
| | | | | 91020447 | 91016041 | | |
| 279 | 84146 | ZNF644 | intron_04 | chr1: | chr1: | GAUUUAGCUGUGUGAGCACU | − |
| | | | | 90982371- | 91015994- | | |
| | | | | 91020447 | 91016013 | | |
| 280 | 84146 | ZNF644 | intron_04 | chr1: | chr1: | AUAACUAUUAUUACUCUAAC | + |
| | | | | 90982371- | 91015859- | | |
| | | | | 91020447 | 91015878 | | |
| 281 | 84146 | ZNF644 | intron_04 | chr1: | chr1: | CAACCUUGUCUUUAUCGGCA | + |
| | | | | 90982371- | 91015740- | | |
| | | | | 91020447 | 91015759 | | |
| 282 | 84146 | ZNF644 | intron_04 | chr1: | chr1: | UAUAACAACCUUGUCUUUAU | + |
| | | | | 90982371- | 91015735- | | |
| | | | | 91020447 | 91015754 | | |
| 283 | 84146 | ZNF644 | intron_04 | chr1: | chr1: | AAGCCAUGCCGAUAAAGACA | − |
| | | | | 90982371- | 91015746- | | |
| | | | | 91020447 | 91015765 | | |
| 284 | 84146 | ZNF644 | intron_04 | chr1: | chr1: | AGCAGCAGAAUCUUAUAACC | − |
| | | | | 90982371- | 91015671- | | |
| | | | | 91020447 | 91015690 | | |
| 285 | 84146 | ZNF644 | intron_04 | chr1: | chr1: | ACUCAUCUGCUGACAGUGCU | + |
| | | | | 90982371- | 91015623- | | |
| | | | | 91020447 | 91015642 | | |
| 286 | 84146 | ZNF644 | intron_04 | chr1: | chr1: | AGCAGAUGAGUAGAUACACU | − |
| | | | | 90982371- | 91015614- | | |
| | | | | 91020447 | 91015633 | | |
| 287 | 84146 | ZNF644 | intron_04 | chr1: | chr1: | GACAAACCAAUGCACUGGCU | + |
| | | | | 90982371- | 91015560- | | |
| | | | | 91020447 | 91015579 | | |
| 288 | 84146 | ZNF644 | intron_04 | chr1: | chr1: | CCACUCCUCACUCUCAUCUU | + |
| | | | | 90982371- | 91015416- | | |
| | | | | 91020447 | 91015435 | | |
| 289 | 84146 | ZNF644 | intron_04 | chr1: | chr1: | GCUGGUAGGUAGCUAGAUUG | − |
| | | | | 90982371- | 91015397- | | |
| | | | | 91020447 | 91015416 | | |
| 290 | 84146 | ZNF644 | intron_04 | chr1: | chr1: | GGUAGGUAGCUAGAUUGAGG | − |
| | | | | 90982371- | 91015394- | | |
| | | | | 91020447 | 91015413 | | |
| 291 | 84146 | ZNF644 | intron_04 | chr1: | chr1: | GGUAGCUAGAUUGAGGCGGG | − |
| | | | | 90982371- | 91015390- | | |
| | | | | 91020447 | 91015409 | | |
| 292 | 84146 | ZNF644 | intron_04 | chr1: | chr1: | AAAUCCGUGUUUGUGCUAGG | + |
| | | | | 90982371- | 91015206- | | |
| | | | | 91020447 | 91015225 | | |
| 293 | 84146 | ZNF644 | intron_04 | chr1: | chr1: | ACCUCCUCCUAGCACAAACA | − |
| | | | | 90982371- | 91015213- | | |
| | | | | 91020447 | 91015232 | | |
| 294 | 84146 | ZNF644 | intron_04 | chr1: | chr1: | CAGCUAGUAAAAAUAAUUUU | − |
| | | | | 90982371- | 91015159- | | |
| | | | | 91020447 | 91015178 | | |
| 295 | 84146 | ZNF644 | intron_04 | chr1: | chr1: | GAUAUACCUAGGUUCAAACU | + |
| | | | | 90982371- | 91014895- | | |
| | | | | 91020447 | 91014914 | | |
| 296 | 84146 | ZNF644 | intron_04 | chr1: | chr1: | GACCUUCUCAAGAUAUACCU | + |
| | | | | 90982371- | 91014884- | | |
| | | | | 91020447 | 91014903 | | |
| 297 | 84146 | ZNF644 | intron_04 | chr1: | chr1: | GAUAUAUAUGUAAGAAGAAC | + |
| | | | | 90982371- | 91014833- | | |
| | | | | 91020447 | 91014852 | | |
| 298 | 84146 | ZNF644 | intron_04 | chr1: | chr1: | CACCACUUGAAAUAAGGGGG | + |
| | | | | 90982371- | 91014734- | | |
| | | | | 91020447 | 91014753 | | |
| 299 | 84146 | ZNF644 | intron_04 | chr1: | chr1: | ACACCACUUGAAAUAAGGGG | + |
| | | | | 90982371- | 91014733- | | |
| | | | | 91020447 | 91014752 | | |
| 300 | 84146 | ZNF644 | intron_04 | chr1: | chr1: | GACACCACUUGAAAUAAGGG | + |
| | | | | 90982371- | 91014732- | | |
| | | | | 91020447 | 91014751 | | |
| 301 | 84146 | ZNF644 | intron_04 | chr1: | chr1: | CGACACCACUUGAAAUAAGG | + |
| | | | | 90982371- | 91014731- | | |
| | | | | 91020447 | 91014750 | | |
| 302 | 84146 | ZNF644 | intron_04 | chr1: | chr1: | ACGACACCACUUGAAAUAAG | + |
| | | | | 90982371- | 91014730- | | |
| | | | | 91020447 | 91014749 | | |
| 303 | 84146 | ZNF644 | intron_04 | chr1: | chr1: | UACGACACCACUUGAAAUAA | + |
| | | | | 90982371- | 91014729- | | |
| | | | | 91020447 | 91014748 | | |
| 304 | 84146 | ZNF644 | intron_04 | chr1: | chr1: | AUACGACACCACUUGAAAUA | + |
| | | | | 90982371- | 91014728- | | |
| | | | | 91020447 | 91014747 | | |
| 305 | 84146 | ZNF644 | intron_04 | chr1: | chr1: | CUCCCCCCCCUUAUUUCAAG | − |
| | | | | 90982371- | 91014739- | | |
| | | | | 91020447 | 91014758 | | |
| 306 | 84146 | ZNF644 | intron_04 | chr1: | chr1: | CUAUUCAUACCACCAUAUAA | − |
| | | | | 90982371- | 91014696- | | |
| | | | | 91020447 | 91014715 | | |
| 307 | 84146 | ZNF644 | intron_04 | chr1: | chr1: | CAUAUAAAGGCUCUUUGUCU | − |
| | | | | 90982371- | 91014683- | | |
| | | | | 91020447 | 91014702 | | |
| 308 | 84146 | ZNF644 | intron_04 | chr1: | chr1: | GUAAAGGGCAGCAUCCAUUU | + |
| | | | | 90982371- | 91014629- | | |
| | | | | 91020447 | 91014648 | | |
| 309 | 84146 | ZNF644 | intron_04 | chr1: | chr1: | AUCAUGCUAUCUAUCCUUGC | − |
| | | | | 90982371- | 91014535- | | |
| | | | | 91020447 | 91014554 | | |
| 310 | 84146 | ZNF644 | intron_04 | chr1: | chr1: | AAAAGCUUAAUGUUGAAUUC | − |
| | | | | 90982371- | 91014304- | | |
| | | | | 91020447 | 91014323 | | |
| 311 | 84146 | ZNF644 | intron_04 | chr1: | chr1: | UGUUGAAUUCUGGCACUCAU | − |
| | | | | 90982371- | 91014294- | | |
| | | | | 91020447 | 91014313 | | |
| 312 | 84146 | ZNF644 | intron_04 | chr1: | chr1: | GUUGAAUUCUGGCACUCAUC | − |
| | | | | 90982371- | 91014293- | | |
| | | | | 91020447 | 91014312 | | |
| 313 | 84146 | ZNF644 | intron_04 | chr1: | chr1: | UUACAGAUAGUAAGUCCUAU | − |
| | | | | 90982371- | 91013685- | | |
| | | | | 91020447 | 91013704 | | |
| 314 | 84146 | ZNF644 | intron_04 | chr1: | chr1: | UACAGAUAGUAAGUCCUAUG | − |
| | | | | 90982371- | 91013684- | | |
| | | | | 91020447 | 91013703 | | |
| 315 | 84146 | ZNF644 | intron_04 | chr1: | chr1: | AUUCUUUCCCUAUACCCCAU | + |
| | | | | 90982371- | 91013667- | | |
| | | | | 91020447 | 91013686 | | |
| 316 | 84146 | ZNF644 | intron_04 | chr1: | chr1: | UAGUAAGUCCUAUGGGGUAU | − |
| | | | | 90982371- | 91013678- | | |
| | | | | 91020447 | 91013697 | | |
| 317 | 84146 | ZNF644 | intron_04 | chr1: | chr1: | AGUAAGUCCUAUGGGGUAUA | − |
| | | | | 90982371- | 91013677- | | |
| | | | | 91020447 | 91013696 | | |
| 318 | 84146 | ZNF644 | intron_04 | chr1: | chr1: | AGCAAAUACAGUUUGAUACC | − |
| | | | | 90982371- | 91013555- | | |
| | | | | 91020447 | 91013574 | | |
| 319 | 84146 | ZNF644 | intron_04 | chr1: | chr1: | AUUCAAGCCACUCUAUUACC | + |
| | | | | 90982371- | 91013534- | | |
| | | | | 91020447 | 91013553 | | |
| 320 | 84146 | ZNF644 | intron_04 | chr1: | chr1: | GAGUGGCUUGAAUCCAGUUU | − |
| | | | | 90982371- | 91013527- | | |
| | | | | 91020447 | 91013546 | | |
| 321 | 84146 | ZNF644 | intron_04 | chr1: | chr1: | UCAUGUAUGAGACACGAAUU | + |
| | | | | 90982371- | 91013397- | | |
| | | | | 91020447 | 91013416 | | |
| 322 | 84146 | ZNF644 | intron_04 | chr1: | chr1: | UAUGACACUACAGAAAAUGC | − |
| | | | | 90982371- | 91013019- | | |
| | | | | 91020447 | 91013038 | | |
| 323 | 84146 | ZNF644 | intron_04 | chr1: | chr1: | UUCCCUUUUGAAGAUCGACU | + |
| | | | | 90982371- | 91012974- | | |
| | | | | 91020447 | 91012993 | | |
| 324 | 84146 | ZNF644 | intron_04 | chr1: | chr1: | AAGCCUAGUCGAUCUUCAAA | − |
| | | | | 90982371- | 91012980- | | |
| | | | | 91020447 | 91012999 | | |
| 325 | 84146 | ZNF644 | intron_04 | chr1: | chr1: | AGCCUAGUCGAUCUUCAAAA | − |
| | | | | 90982371- | 91012979- | | |
| | | | | 91020447 | 91012998 | | |
| 326 | 84146 | ZNF644 | intron_04 | chr1: | chr1: | UUCUGUGAUUAACAAUAUGA | − |
| | | | | 90982371- | 91012928- | | |
| | | | | 91020447 | 91012947 | | |
| 327 | 84146 | ZNF644 | intron_04 | chr1: | chr1: | CCACUACCUGGGAGUUACCU | + |
| | | | | 90982371- | 91012752- | | |
| | | | | 91020447 | 91012771 | | |
| 328 | 84146 | ZNF644 | intron_04 | chr1: | chr1: | CCCAGUACCUCCCACUACCU | + |
| | | | | 90982371- | 91012741- | | |
| | | | | 91020447 | 91012760 | | |
| 329 | 84146 | ZNF644 | intron_04 | chr1: | chr1: | ACCCAGUACCUCCCACUACC | + |
| | | | | 90982371- | 91012740- | | |
| | | | | 91020447 | 91012759 | | |
| 330 | 84146 | ZNF644 | intron_04 | chr1: | chr1: | GGAGGUACUGGGUUACACCU | − |
| | | | | 90982371- | 91012733- | | |
| | | | | 91020447 | 91012752 | | |
| 331 | 84146 | ZNF644 | intron_04 | chr1: | chr1: | GAGGUACUGGGUUACACCUG | − |
| | | | | 90982371- | 91012732- | | |
| | | | | 91020447 | 91012751 | | |
| 332 | 84146 | ZNF644 | intron_04 | chr1: | chr1: | GGGUUACACCUGGGGAGAAG | − |
| | | | | 90982371- | 91012724- | | |
| | | | | 91020447 | 91012743 | | |
| 333 | 84146 | ZNF644 | intron_04 | chr1: | chr1: | AAUUAAUAGGAGGGAGCACA | + |
| | | | | 90982371- | 91012578- | | |
| | | | | 91020447 | 91012597 | | |
| 334 | 84146 | ZNF644 | intron_04 | chr1: | chr1: | GUAGGGAGAAAUUAAUAGGA | + |
| | | | | 90982371- | 91012569- | | |
| | | | | 91020447 | 91012588 | | |
| 335 | 84146 | ZNF644 | intron_04 | chr1: | chr1: | UUCUGUAGGGAGAAAUUAAU | + |
| | | | | 90982371- | 91012565- | | |
| | | | | 91020447 | 91012584 | | |
| 336 | 84146 | ZNF644 | intron_04 | chr1: | chr1: | GAGGUUAGAGGUUAAAAUAG | + |
| | | | | 90982371- | 91012538- | | |
| | | | | 91020447 | 91012557 | | |
| 337 | 84146 | ZNF644 | intron_04 | chr1: | chr1: | CAAUUCUCACUACCCUUGGG | + |
| | | | | 90982371- | 91012209- | | |
| | | | | 91020447 | 91012228 | | |
| 338 | 84146 | ZNF644 | intron_04 | chr1: | chr1: | UGUUUGGCAUUGUUAUGAAU | − |
| | | | | 90982371- | 91012156- | | |
| | | | | 91020447 | 91012175 | | |
| 339 | 84146 | ZNF644 | intron_04 | chr1: | chr1: | GAGGUAAAUCAGGUGAUAUU | − |
| | | | | 90982371- | 91012133- | | |
| | | | | 91020447 | 91012152 | | |
| 340 | 84146 | ZNF644 | intron_04 | chr1: | chr1: | UCAGGUGAUAUUUGGUACUA | − |
| | | | | 90982371- | 91012125- | | |
| | | | | 91020447 | 91012144 | | |
| 341 | 84146 | ZNF644 | intron_04 | chr1: | chr1: | CUCUCUGAUUUUGUCACUGG | + |
| | | | | 90982371- | 91012089- | | |
| | | | | 91020447 | 91012108 | | |
| 342 | 84146 | ZNF644 | intron_04 | chr1: | chr1: | ACCUCUCUGAUUUUGUCACU | + |
| | | | | 90982371- | 91012087- | | |
| | | | | 91020447 | 91012106 | | |
| 343 | 84146 | ZNF644 | intron_04 | chr1: | chr1: | CAUUAAUAGUAAUCCUUUAG | − |
| | | | | 90982371- | 91012054- | | |
| | | | | 91020447 | 91012073 | | |
| 344 | 84146 | ZNF64 | intron_04 | chr1: | chr1: | GUCAUAAAACUAACCUCUAA | + |
| | | | | 90982371- | 91012038- | | |
| | | | | 91020447 | 91012057 | | |
| 345 | 84146 | ZNF644 | intron_04 | chr1: | chr1: | GGCAACUAUGUUAUCGGUGG | + |
| | | | | 90982371- | 91011979- | | |
| | | | | 91020447 | 91011998 | | |
| 346 | 84146 | ZNF644 | intron_04 | chr1: | chr1: | CCAGGCAACUAUGUUAUCGG | + |
| | | | | 90982371- | 91011976- | | |
| | | | | 91020447 | 91011995 | | |
| 347 | 84146 | ZNF644 | intron_04 | chr1: | chr1: | AAGCCAGGCAACUAUGUUAU | + |
| | | | | 90982371- | 91011973- | | |
| | | | | 91020447 | 91011992 | | |
| 348 | 84146 | ZNF644 | intron_04 | chr1: | chr1: | CCACCGAUAACAUAGUUGCC | − |
| | | | | 90982371- | 91011979- | | |
| | | | | 91020447 | 91011998 | | |
| 349 | 84146 | ZNF644 | intron_04 | chr1: | chr1: | UAUUCUCUAGGCAUAUAGCC | − |
| | | | | 90982371- | 91011940- | | |
| | | | | 91020447 | 91011959 | | |
| 350 | 84146 | ZNF644 | intron_04 | chr1: | chr1: | AACUUCCUCCACCUACUUCC | + |
| | | | | 90982371- | 91011919- | | |
| | | | | 91020447 | 91011938 | | |
| 351 | 84146 | ZNF644 | intron_04 | chr1: | chr1: | UAGGCAUAUAGCCAGGAAGU | − |
| | | | | 90982371- | 91011933- | | |
| | | | | 91020447 | 91011952 | | |
| 352 | 84146 | ZNF644 | intron_04 | chr1: | chr1: | GCAUAUAGCCAGGAAGUAGG | − |
| | | | | 90982371- | 91011930- | | |
| | | | | 91020447 | 91011949 | | |
| 353 | 84146 | ZNF644 | intron_04 | chr1: | chr1: | UAUAGCCAGGAAGUAGGUGG | − |
| | | | | 90982371- | 91011927- | | |
| | | | | 91020447 | 91011946 | | |
| 354 | 84146 | ZNF644 | intron_04 | chr1: | chr1: | CUCCAAAAACCAUCUAUAUA | + |
| | | | | 90982371- | 91011885- | | |
| | | | | 91020447 | 91011904 | | |
| 355 | 84146 | ZNF644 | intron_04 | chr1: | chr1: | GGAUUGCCUCCUUAUAUAGA | − |
| | | | | 90982371- | 91011897- | | |
| | | | | 91020447 | 91011916 | | |
| 356 | 84146 | ZNF644 | intron_04 | chr1: | chr1: | CCUCUUUGAUGGUAGCUUCC | − |
| | | | | 90982371- | 91011865- | | |
| | | | | 91020447 | 91011884 | | |
| 357 | 84146 | ZNF644 | intron_04 | chr1: | chr1: | UAGCUAACUGACAUAAUGCC | + |
| | | | | 90982371- | 91011844- | | |
| | | | | 91020447 | 91011863 | | |
| 358 | 84146 | ZNF644 | intron_04 | chr1: | chr1: | CAUUAUGUCAGUUAGCUAUU | − |
| | | | | 90982371- | 91011842- | | |
| | | | | 91020447 | 91011861 | | |
| 359 | 84146 | ZNF644 | intron_04 | chr1: | chr1: | AGUUAGCUAUUAGGCAUUUU | − |
| | | | | 90982371- | 91011833- | | |
| | | | | 91020447 | 91011852 | | |
| 360 | 84146 | ZNF644 | intron_04 | chr1: | chr1: | GCAUUUUAGGUUGCUUAUUA | − |
| | | | | 90982371- | 91011820- | | |
| | | | | 91020447 | 91011839 | | |
| 361 | 84146 | ZNF644 | intron_04 | chr1: | chr1: | AGGUUGCUUAUUAAGGUAUU | − |
| | | | | 90982371- | 91011813- | | |
| | | | | 91020447 | 91011832 | | |
| 362 | 84146 | ZNF644 | intron_04 | chr1: | chr1: | GGUUGCUUAUUAAGGUAUUU | − |
| | | | | 90982371- | 91011812- | | |
| | | | | 91020447 | 91011831 | | |
| 363 | 84146 | ZNF644 | intron_04 | chr1: | chr1: | CUGAUUGUAAUUUUAGAGGU | − |
| | | | | 90982371- | 91011784- | | |
| | | | | 91020447 | 91011803 | | |
| 364 | 84146 | ZNF644 | intron_04 | chr1: | chr1: | UUUUAGAGGUAGGAAUAAAU | − |
| | | | | 90982371- | 91011774- | | |
| | | | | 91020447 | 91011793 | | |
| 365 | 84146 | ZNF644 | intron_04 | chr1: | chr1: | CUUGUAAGCAACUGUCCAUA | + |
| | | | | 90982371- | 91011681- | | |
| | | | | 91020447 | 91011700 | | |
| 366 | 84146 | ZNF644 | intron_04 | chr1: | chr1: | CAGUUGCUUACAAGUGUGAA | − |
| | | | | 90982371- | 91011675- | | |
| | | | | 91020447 | 91011694 | | |
| 367 | 84146 | ZNF644 | intron_04 | chr1: | chr1: | UGAAUGGUUAAAGCCCAUGU | − |
| | | | | 90982371- | 91011659- | | |
| | | | | 91020447 | 91011678 | | |
| 368 | 84146 | ZNF644 | intron_04 | chr1: | chr1: | CAACUAUUAAUUCCAUUUGG | + |
| | | | | 90982371- | 91011558- | | |
| | | | | 91020447 | 91011577 | | |
| 369 | 84146 | ZNF644 | intron_04 | chr1: | chr1: | ACAACUAUUAAUUCCAUUUG | + |
| | | | | 90982371- | 91011557- | | |
| | | | | 91020447 | 91011576 | | |
| 370 | 84146 | ZNF644 | intron_04 | chr1: | chr1: | GUAGUAUAGCAGUAUUAAAU | − |
| | | | | 90982371- | 91011496- | | |
| | | | | 91020447 | 91011515 | | |
| 371 | 84146 | ZNF644 | intron_04 | chr1: | chr1: | AUAUGUAAAUUGCGCAGAAA | − |
| | | | | 90982371- | 91011365- | | |
| | | | | 91020447 | 91011384 | | |
| 372 | 84146 | ZNF644 | intron_04 | chr1: | chr1: | AUUUAAUAUACUCUUGCUCC | − |
| | | | | 90982371- | 91011232- | | |
| | | | | 91020447 | 91011251 | | |
| 373 | 84146 | ZNF644 | intron_04 | chr1: | chr1: | CUUUACACAUAACAGCUUUU | − |
| | | | | 90982371- | 91011062- | | |
| | | | | 91020447 | 91011081 | | |
| 374 | 84146 | ZNF644 | intron_04 | chr1: | chr1: | UUACACAUAACAGCUUUUGG | − |
| | | | | 90982371- | 91011060- | | |
| | | | | 91020447 | 91011079 | | |
| 375 | 84146 | ZNF644 | intron_04 | chr1: | chr1: | UCAGAAAAGUCUUGUUUGUC | − |
| | | | | 90982371- | 91010862- | | |
| | | | | 91020447 | 91010881 | | |
| 376 | 84146 | ZNF644 | intron_04 | chr1: | chr1: | AUUCAGCUAAUGCUCCACUA | − |
| | | | | 90982371- | 91010783- | | |
| | | | | 91020447 | 91010802 | | |
| 377 | 84146 | ZNF644 | intron_04 | chr1: | chr1: | UUCAGCUAAUGCUCCACUAG | − |
| | | | | 90982371- | 91010782- | | |
| | | | | 91020447 | 91010801 | | |
| 378 | 84146 | ZNF644 | intron_04 | chr1: | chr1: | GCUAAUGCUCCACUAGGGGU | − |
| | | | | 90982371- | 91010778- | | |
| | | | | 91020447 | 91010797 | | |
| 379 | 84146 | ZNF644 | intron_04 | chr1: | chr1: | CUAAUGCUCCACUAGGGGUA | − |
| | | | | 90982371- | 91010777- | | |
| | | | | 91020447 | 91010796 | | |
| 380 | 84146 | ZNF644 | intron_04 | chr1: | chr1: | AUCUUAGAUCAUGAGCUCUU | + |
| | | | | 90982371- | 91010716- | | |
| | | | | 91020447 | 91010735 | | |
| 381 | 84146 | ZNF644 | intron_04 | chr1: | chr1: | AAUAAGAUUGUGGCAUGAGC | − |
| | | | | 90982371- | 91010141- | | |
| | | | | 91020447 | 91010160 | | |
| 382 | 84146 | ZNF644 | intron_04 | chr1: | chr1: | CCCUCCAAUCACGCUGUAUC | + |
| | | | | 90982371- | 91010023- | | |
| | | | | 91020447 | 91010042 | | |
| 383 | 84146 | ZNF644 | intron_04 | chr1: | chr1: | GCAGCCAGAUACAGCGUGAU | − |
| | | | | 90982371- | 91010030- | | |
| | | | | 91020447 | 91010049 | | |
| 384 | 84146 | ZNF644 | intron_04 | chr1: | chr1: | GCCAGAUACAGCGUGAUUGG | − |
| | | | | 90982371- | 91010027- | | |
| | | | | 91020447 | 91010046 | | |
| 385 | 84146 | ZNF644 | intron_04 | chr1: | chr1: | CCAGAUACAGCGUGAUUGGA | − |
| | | | | 90982371- | 91010026- | | |
| | | | | 91020447 | 91010045 | | |
| 386 | 84146 | ZNF644 | intron_04 | chr1: | chr1: | UAUUAUCUUUGUGAGUGGGA | − |
| | | | | 90982371- | 91009973- | | |
| | | | | 91020447 | 91009992 | | |
| 387 | 84146 | ZNF644 | intron_04 | chr1: | chr1: | GGAAGGGUAUAUUAAGAGUA | − |
| | | | | 90982371- | 91009956- | | |
| | | | | 91020447 | 91009975 | | |
| 388 | 84146 | ZNF644 | intron_04 | chr1: | chr1: | UAGCUCUGCACUACAAACUG | + |
| | | | | 90982371- | 91009891- | | |
| | | | | 91020447 | 91009910 | | |
| 389 | 84146 | ZNF644 | intron_04 | chr1: | chr1: | UGACUUACUAGCACUAAUUG | − |
| | | | | 90982371- | 91009806- | | |
| | | | | 91020447 | 91009825 | | |
| 390 | 84146 | ZNF644 | intron_04 | chr1: | chr1: | UUAUAAUGUCUCUUAAGCCA | + |
| | | | | 90982371- | 91009607- | | |
| | | | | 91020447 | 91009626 | | |
| 391 | 84146 | ZNF644 | intron_04 | chr1: | chr1: | AUUAUAAUUUGUAUUGGAGC | − |
| | | | | 90982371- | 91009594- | | |
| | | | | 91020447 | 91009613 | | |
| 392 | 84146 | ZNF644 | intron_04 | chr1: | chr1: | AUUGGAGCUGGCAGGAAAAC | − |
| | | | | 90982371- | 91009582- | | |
| | | | | 91020447 | 91009601 | | |
| 393 | 84146 | ZNF644 | intron_04 | chr1: | chr1: | UGGGAAUACUCUGCUUUUAU | − |
| | | | | 90982371- | 91009479- | | |
| | | | | 91020447 | 91009498 | | |
| 394 | 84146 | ZNF644 | intron_04 | chr1: | chr1: | CCAUAAGGUACACUCAAUUU | + |
| | | | | 90982371- | 91009416- | | |
| | | | | 91020447 | 91009435 | | |
| 395 | 84146 | ZNF644 | intron_04 | chr1: | chr1: | CCUAAAUUGAGUGUACCUUA | − |
| | | | | 90982371- | 91009419- | | |
| | | | | 91020447 | 91009438 | | |
| 396 | 84146 | ZNF644 | intron_04 | chr1: | chr1: | UUUACCCAUAAUAAACCAUA | + |
| | | | | 90982371- | 91009401- | | |
| | | | | 91020447 | 91009420 | | |
| 397 | 84146 | ZNF644 | intron_04 | chr1: | chr1: | GUGUACCUUAUGGUUUAUUA | − |
| | | | | 90982371- | 91009409- | | |
| | | | | 91020447 | 91009428 | | |
| 398 | 84146 | ZNF644 | intron_04 | chr1: | chr1: | UGUACCUUAUGGUUUAUUAU | − |
| | | | | 90982371- | 91009408- | | |
| | | | | 91020447 | 91009427 | | |
| 399 | 84146 | ZNF644 | intron_04 | chr1: | chr1: | UAGGAAAUAGAACAGAAGCU | − |
| | | | | 90982371- | 91009298- | | |
| | | | | 91020447 | 91009317 | | |
| 400 | 84146 | ZNF644 | intron_04 | chr1: | chr1: | GUAAAUGUUAUACUAAAACG | + |
| | | | | 90982371- | 91009244- | | |
| | | | | 91020447 | 91009263 | | |
| 401 | 84146 | ZNF644 | intron_04 | chr1: | chr1: | CAGUUAAUAUAGGCUUAACU | − |
| | | | | 90982371- | 91009208- | | |
| | | | | 91020447 | 91009227 | | |
| 402 | 84146 | ZNF644 | intron_04 | chr1: | chr1: | AGUAUGUAGUAGUUCUCUGC | − |
| | | | | 90982371- | 91009163- | | |
| | | | | 91020447 | 91009182 | | |
| 403 | 84146 | ZNF644 | intron_04 | chr1: | chr1: | CCUGUUGGAAAGGAGCGAAG | + |
| | | | | 90982371- | 91009125- | | |
| | | | | 91020447 | 91009144 | | |
| 404 | 84146 | ZNF644 | intron_04 | chr1: | chr1: | ACCUGUUGGAAAGGAGCGAA | + |
| | | | | 90982371- | 91009124- | | |
| | | | | 91020447 | 91009143 | | |
| 405 | 84146 | ZNF644 | intron_04 | chr1: | chr1: | UUGGGCAAUACCUGUUGGAA | + |
| | | | | 90982371- | 91009115- | | |
| | | | | 91020447 | 91009134 | | |
| 406 | 84146 | ZNF644 | intron_04 | chr1: | chr1: | CCCCUUCGCUCCUUUCCAAC | − |
| | | | | 90982371- | 91009128- | | |
| | | | | 91020447 | 91009147 | | |
| 407 | 84146 | ZNF644 | intron_04 | chr1: | chr1: | AGAGCUUGGGCAAUACCUGU | + |
| | | | | 90982371- | 91009110- | | |
| | | | | 91020447 | 91009129 | | |
| 408 | 84146 | ZNF644 | intron_04 | chr1: | chr1: | AGAACGGCUUAUGAGAGCUU | + |
| | | | | 90982371- | 91009097- | | |
| | | | | 91020447 | 91009116 | | |
| 409 | 84146 | ZNF644 | intron_04 | chr1: | chr1: | AAGAACGGCUUAUGAGAGCU | + |
| | | | | 90982371- | 91009096- | | |
| | | | | 91020447 | 91009115 | | |
| 410 | 84146 | ZNF644 | intron_04 | chr1: | chr1: | GAUGAAGUCCUUUCCAGCUU | L |
| | | | | 90982371- | 91008909- | | |
| | | | | 91020447 | 91008928 | | |
| 411 | 84146 | ZNF644 | intron_04 | chr1: | chr1: | UUGACAGCAGGAAUAACAGA | + |
| | | | | 90982371- | 91008875- | | |
| | | | | 91020447 | 91008894 | | |
| 412 | 84146 | ZNF644 | intron_04 | chr1: | chr1: | UCUACUGUGCUUUUCAACCU | − |
| | | | | 90982371- | 91008844- | | |
| | | | | 91020447 | 91008863 | | |
| 413 | 84146 | ZNF644 | intron_04 | chr1: | chr1: | GGUAAUAAUCGCACAGAUGA | + |
| | | | | 90982371- | 91008804- | | |
| | | | | 91020447 | 91008823 | | |
| 414 | 84146 | ZNF644 | intron_04 | chr1: | chr1: | CUUUACUUGCUGCAGUUAAA | + |
| | | | | 90982371- | 91008783- | | |
| | | | | 91020447 | 91008802 | | |
| 415 | 84146 | ZNF644 | intron_04 | chr1: | chr1: | AAGUAAAGACGUUAAUAGUG | − |
| | | | | 90982371- | 91008771- | | |
| | | | | 91020447 | 91008790 | | |
| 416 | 84146 | ZNF644 | intron_04 | chr1: | chr1: | ACGUUAAUAGUGAGGUUUUU | − |
| | | | | 90982371- | 91008763- | | |
| | | | | 91020447 | 91008782 | | |
| 417 | 84146 | ZNF644 | intron_04 | chr1: | chr1: | UAGUGAGGUUUUUGGGAAUG | − |
| | | | | 90982371- | 91008756- | | |
| | | | | 91020447 | 91008775 | | |
| 418 | 84146 | ZNF644 | intron_04 | chr1: | chr1: | GGGAAUGUGGUAAAACCGGG | − |
| | | | | 90982371- | 91008743- | | |
| | | | | 91020447 | 91008762 | | |
| 419 | 84146 | ZNF644 | intron_04 | chr1: | chr1: | UUUGUCCAAGUUAUCUGAUG | − |
| | | | | 90982371- | 91008710- | | |
| | | | | 91020447 | 91008729 | | |
| 420 | 84146 | ZNF644 | intron_04 | chr1: | chr1: | UCAGCUAAAGCAAAAUACAG | − |
| | | | | 90982371- | 91008683- | | |
| | | | | 91020447 | 91008702 | | |
| 421 | 84146 | ZNF644 | intron_04 | chr1: | chr1: | UCUAGGUCCCAGUGAUAGUA | + |
| | | | | 90982371- | 91008651- | | |
| | | | | 91020447 | 91008670 | | |
| 422 | 84146 | ZNF644 | intron_04 | chr1: | chr1: | GGGUUGCUCCCUACUAUCAC | − |
| | | | | 90982371- | 91008662- | | |
| | | | | 91020447 | 91008681 | | |
| 423 | 84146 | ZNF644 | intron_04 | chr1: | chr1: | GGUUGCUCCCUACUAUCACU | − |
| | | | | 90982371- | 91008661- | | |
| | | | | 91020447 | 91008680 | | |
| 424 | 84146 | ZNF644 | intron_04 | chr1: | chr1: | UCAUCUACAUCUCUGAAAAA | − |
| | | | | 90982371- | 91008627- | | |
| | | | | 91020447 | 91008646 | | |
| 425 | 84146 | ZNF644 | intron_04 | chr1: | chr1: | GGGUUUCUGUAUGAUAGUAU | − |
| | | | | 90982371- | 91008605- | | |
| | | | | 91020447 | 91008624 | | |
| 426 | 84146 | ZNF644 | intron_04 | chr1: | chr1: | UCCUAUACCACUAGUUCAUC | + |
| | | | | 90982371- | 91008536- | | |
| | | | | 91020447 | 91008555 | | |
| 427 | 84146 | ZNF644 | intron_04 | chr1: | chr1: | UCCUGAUGAACUAGUGGUAU | − |
| | | | | 90982371- | 91008540- | | |
| | | | | 91020447 | 91008559 | | |
| 428 | 84146 | ZNF644 | intron_04 | chr1: | chr1: | AGAACUCUGGAGCCAUACAC | + |
| | | | | 90982371- | 91008458- | | |
| | | | | 91020447 | 91008477 | | |
| 429 | 84146 | ZNF644 | intron_04 | chr1: | chr1: | GUGUAUGGCUCCAGAGUUCU | − |
| | | | | 90982371- | 91008458- | | |
| | | | | 91020447 | 91008477 | | |
| 430 | 84146 | ZNF644 | intron_04 | chr1: | chr1: | ACCAGUUCUAGACUAAUUCU | + |
| | | | | 90982371- | 91008365- | | |
| | | | | 91020447 | 91008384 | | |
| 431 | 84146 | ZNF644 | intron_04 | chr1: | chr1: | UCUCAAGUACAGAGAUCCUC | + |
| | | | | 90982371- | 91008195- | | |
| | | | | 91020447 | 91008214 | | |
| 432 | 84146 | ZNF644 | intron_04 | chr1: | chr1: | CAGUAUUCUCAGAACUGGGA | + |
| | | | | 90982371- | 91008072- | | |
| | | | | 91020447 | 91008091 | | |
| 433 | 84146 | ZNF644 | intron_04 | chr1: | chr1: | UCAGCCAGUAUUCUCAGAAC | + |
| | | | | 90982371- | 91008067- | | |
| | | | | 91020447 | 91008086 | | |
| 434 | 84146 | ZNF644 | intron_04 | chr1: | chr1: | UACUGUAGGAUUAUCAAUCU | + |
| | | | | 90982371- | 91007947- | | |
| | | | | 91020447 | 91007966 | | |
| 435 | 84146 | ZNF644 | intron_04 | chr1: | chr1: | AUACUGUAGGAUUAUCAAUC | + |
| | | | | 90982371- | 91007946- | | |
| | | | | 91020447 | 91007965 | | |
| 436 | 84146 | ZNF644 | intron_04 | chr1: | chr1: | UAAUCUCAUAUUUAAGAUUG | + |
| | | | | 90982371- | 91007882- | | |
| | | | | 91020447 | 91007901 | | |
| 437 | 84146 | ZNF644 | intron_04 | chr1: | chr1: | UCUAGAAGCUCUUGUUUUCU | + |
| | | | | 90982371- | 91007699- | | |
| | | | | 91020447 | 91007718 | | |
| 438 | 84146 | ZNF644 | intron_04 | chr1: | chr1: | CUCUUUUUGUCCUCCUCCGU | + |
| | | | | 90982371- | 91007589- | | |
| | | | | 91020447 | 91007608 | | |
| 439 | 84146 | ZNF644 | intron_04 | chr1: | chr1: | ACUCUUUUUGUCCUCCUCCG | + |
| | | | | 90982371- | 91007588- | | |
| | | | | 91020447 | 91007607 | | |
| 440 | 84146 | ZNF644 | intron_04 | chr1: | chr1: | AUUCAUGAGCCCUUUCACUG | + |
| | | | | 90982371- | 91007148- | | |
| | | | | 91020447 | 91007167 | | |
| 441 | 84146 | ZNF644 | intron_04 | chr1: | chr1: | CUUGAUCUUCCCUUUAUUUC | + |
| | | | | 90982371- | 91007062- | | |
| | | | | 91020447 | 91007081 | | |
| 442 | 84146 | ZNF644 | intron_04 | chr1: | chr1: | AAGGUAUAGAAUGAAUAGUU | + |
| | | | | 90982371- | 91006935- | | |
| | | | | 91020447 | 91006954 | | |
| 443 | 84146 | ZNF644 | intron_04 | chr1: | chr1: | CCACACUUGAUAGCUUGGUA | + |
| | | | | 90982371- | 91006916- | | |
| | | | | 91020447 | 91006935 | | |
| 444 | 84146 | ZNF644 | intron_04 | chr1: | chr1: | UCCUCCCACACUUGAUAGCU | + |
| | | | | 90982371- | 91006911- | | |
| | | | | 91020447 | 91006930 | | |
| 445 | 84146 | ZNF644 | intron_04 | chr1: | chr1: | UAGUUAACCCAUCACUUUGA | + |
| | | | | 90982371- | 91006807- | | |
| | | | | 91020447 | 91006826 | | |
| 446 | 84146 | ZNF644 | intron_04 | chr1: | chr1: | CUGUAAUGCAUCCUACUUUC | + |
| | | | | 90982371- | 91006770- | | |
| | | | | 91020447 | 91006789 | | |
| 447 | 84146 | ZNF644 | intron_04 | chr1: | chr1: | AGACUGCUUCCCUGGAAUCU | + |
| | | | | 90982371- | 91006436- | | |
| | | | | 91020447 | 91006455 | | |
| 448 | 84146 | ZNF644 | intron_04 | chr1: | chr1: | CAGACUGCUUCCCUGGAAUC | + |
| | | | | 90982371- | 91006435- | | |
| | | | | 91020447 | 91006454 | | |
| 449 | 84146 | ZNF644 | intron_04 | chr1: | chr1: | AGAUUUAGAUCACUACAGUG | + |
| | | | | 90982371- | 91006019- | | |
| | | | | 91020447 | 91006038 | | |
| 450 | 84146 | ZNF644 | intron_04 | chr1: | chr1: | AAGUAAACGCAAUGCUUACA | + |
| | | | | 90982371- | 91005764- | | |
| | | | | 91020447 | 91005783 | | |
| 451 | 84146 | ZNF644 | intron_04 | chr1: | chr1: | GAAGUAAACGCAAUGCUUAC | + |
| | | | | 90982371- | 91005763- | | |
| | | | | 91020447 | 91005782 | | |
| 452 | 84146 | ZNF644 | intron_04 | chr1: | chr1: | CACCUAACAACAGCAGAGUA | + |
| | | | | 90982371- | 91005476- | | |
| | | | | 91020447 | 91005495 | | |
| 453 | 84146 | ZNF644 | intron_04 | chr1: | chr1: | AGGCAGCACUACAGGACUAG | + |
| | | | | 90982371- | 91004894- | | |
| | | | | 91020447 | 91004913 | | |
| 454 | 84146 | ZNF644 | intron_04 | chr1: | chr1: | UAACAGCACGAAGGAAGGAG | + |
| | | | | 90982371- | 91004686- | | |
| | | | | 91020447 | 91004705 | | |
| 455 | 84146 | ZNF644 | intron_04 | chr1: | chr1: | GGUAUAAAAGGAUGUAUGUA | + |
| | | | | 90982371- | 91004660- | | |
| | | | | 91020447 | 91004679 | | |
| 456 | 84146 | ZNF644 | intron_04 | chr1: | chr1: | AAGCUCAAGGAACUACACAG | + |
| | | | | 90982371- | 91004211- | | |
| | | | | 91020447 | 91004230 | | |
| 457 | 84146 | ZNF644 | intron_04 | chr1: | chr1: | AAUAGGAGUUCUACAAAAGA | + |
| | | | | 90982371- | 91004098- | | |
| | | | | 91020447 | 91004117 | | |
| 458 | 84146 | ZNF644 | intron_04 | chr1: | chr1: | CAUCCCAUUGUAUACCUAAU | + |
| | | | | 90982371- | 91004081- | | |
| | | | | 91020447 | 91004100 | | |
| 459 | 84146 | ZNF644 | intron_04 | chr1: | chr1: | CUACGUCAUCUACAUCAUCA | + |
| | | | | 90982371- | 90998544- | | |
| | | | | 91020447 | 90998563 | | |
| 460 | 84146 | ZNF644 | intron_04 | chr1: | chr1: | CAAACAACGCUGGGGCAGAG | + |
| | | | | 90982371- | 90997463- | | |
| | | | | 91020447 | 90997482 | | |
| 461 | 84146 | ZNF644 | intron_04 | chr1: | chr1: | CUAAAAUAACAAACAACGCU | + |
| | | | | 90982371- | 90997454- | | |
| | | | | 91020447 | 90997473 | | |
| 462 | 84146 | ZNF644 | intron_04 | chr1: | chr1: | ACUAAAAUAACAAACAACGC | + |
| | | | | 90982371- | 90997453- | | |
| | | | | 91020447 | 90997472 | | |
| 463 | 84146 | ZNF644 | intron_04 | chr1: | chr1: | CAAAAAGAUUUAUUGCUCCC | + |
| | | | | 90982371- | 90997311- | | |
| | | | | 91020447 | 90997330 | | |
| 464 | 84146 | ZNF644 | intron_04 | chr1: | chr1: | AAGACCUAAGUGCUCACCUC | + |
| | | | | 90982371- | 90997185- | | |
| | | | | 91020447 | 90997204 | | |
| 465 | 84146 | ZNF644 | intron_04 | chr1: | chr1: | AAAGGGCUGUAUACACGCCC | + |
| | | | | 90982371- | 90997161- | | |
| | | | | 91020447 | 90997180 | | |
| 466 | 84146 | ZNF644 | intron_04 | chr1: | chr1: | AGAGAGCCACAUGUACAGAA | + |
| | | | | 90982371- | 90997143- | | |
| | | | | 91020447 | 90997162 | | |
| 467 | 84146 | ZNF644 | intron_04 | chr1: | chr1: | UAAUUGUUUCAUAUUGCAGC | + |
| | | | | 90982371- | 90997022- | | |
| | | | | 91020447 | 90997041 | | |
| 468 | 84146 | ZNF644 | intron_04 | chr1: | chr1: | CAAUACCCUAGGACAACCAA | + |
| | | | | 90982371- | 90996975- | | |
| | | | | 91020447 | 90996994 | | |
| 469 | 84146 | ZNF644 | intron_04 | chr1: | chr1: | AAAGCUUCCCCCAAUACCCU | + |
| | | | | 90982371- | 90996964- | | |
| | | | | 91020447 | 90996983 | | |
| 470 | 84146 | ZNF644 | intron_04 | chr1: | chr1: | ACAUCUGGUGGUCUCCUGGA | + |
| | | | | 90982371- | 90996887- | | |
| | | | | 91020447 | 90996906 | | |
| 471 | 84146 | ZNF644 | intron_04 | chr1: | chr1: | UCAUUAUUUAACACAUCUGG | + |
| | | | | 90982371- | 90996875- | | |
| | | | | 91020447 | 90996894 | | |
| 472 | 84146 | ZNF644 | intron_04 | chr1: | chr1: | AAUUAGCAGCCUGGGGGAUC | + |
| | | | | 90982371- | 90996487- | | |
| | | | | 91020447 | 90996506 | | |
| 473 | 84146 | ZNF644 | intron_04 | chr1: | chr1: | CUCAGUCACAUUUGGAAUCA | + |
| | | | | 90982371- | 90996099- | | |
| | | | | 91020447 | 90996118 | | |
| 474 | 84146 | ZNF644 | intron_04 | chr1: | chr1: | ACUCAGUCACAUUUGGAAUC | + |
| | | | | 90982371- | 90996098- | | |
| | | | | 91020447 | 90996117 | | |
| 475 | 84146 | ZNF644 | intron_04 | chr1: | chr1: | GGUUGACCUAUCAGAAAAAA | + |
| | | | | 90982371- | 90995681- | | |
| | | | | 91020447 | 90995700 | | |
| 476 | 84146 | ZNF644 | intron_04 | chr1: | chr1: | CAUAUAUAUCAUAGUCAAGC | + |
| | | | | 90982371- | 90995581- | | |
| | | | | 91020447 | 90995600 | | |
| 477 | 84146 | ZNF644 | intron_04 | chr1: | chr1: | UAUGUAAGUCCCAAAAAGAA | + |
| | | | | 90982371- | 90995423- | | |
| | | | | 91020447 | 90995442 | | |
| 478 | 84146 | ZNF644 | intron_04 | chr1: | chr1: | GAGACCAGUAGGACACUAUU | + |
| | | | | 90982371- | 90995388- | | |
| | | | | 91020447 | 90995407 | | |
| 479 | 84146 | ZNF644 | intron_04 | chr1: | chr1: | AUACCAUAAUGUGCAGUUUC | + |
| | | | | 90982371- | 90995017- | | |
| | | | | 91020447 | 90995036 | | |
| 480 | 84146 | ZNF644 | intron_04 | chr1: | chr1: | GGGAGACCAAGUUUGCAGUC | + |
| | | | | 90982371- | 90994917- | | |
| | | | | 91020447 | 90994936 | | |
| 481 | 84146 | ZNF644 | intron_04 | chr1: | chr1: | GCCGUGGCUGCAUACAUCAG | + |
| | | | | 90982371- | 90994897- | | |
| | | | | 91020447 | 90994916 | | |
| 482 | 84146 | ZNF644 | intron_04 | chr1: | chr1: | GGCCGUGGCUGCAUACAUCA | + |
| | | | | 90982371- | 90994896- | | |
| | | | | 91020447 | 90994915 | | |
| 483 | 84146 | ZNF644 | intron_04 | chr1: | chr1: | UGGCCGUGGCUGCAUACAUC | + |
| | | | | 90982371- | 90994895- | | |
| | | | | 91020447 | 90994914 | | |
| 484 | 84146 | ZNF644 | intron_04 | chr1: | chr1: | CAACCAACUUACUCAGACAC | + |
| | | | | 90982371- | 90994813- | | |
| | | | | 91020447 | 90994832 | | |
| 485 | 84146 | ZNF644 | intron_04 | chr1: | chr1: | CUACAAUCUCCACAAAUCUC | + |
| | | | | 90982371- | 90994786- | | |
| | | | | 91020447 | 90994805 | | |
| 486 | 84146 | ZNF644 | intron_04 | chr1: | chr1: | CCGAGAAGCAAGAAAACCAC | + |
| | | | | 90982371- | 90994752- | | |
| | | | | 91020447 | 90994771 | | |
| 487 | 84146 | ZNF644 | intron_04 | chr1: | chr1: | CAAGGUUGAAGUACUAGUGA | + |
| | | | | 90982371- | 90994691- | | |
| | | | | 91020447 | 90994710 | | |
| 488 | 84146 | ZNF644 | intron_04 | chr1: | chr1: | CAGGAAGCUGCAGCUCUACA | + |
| | | | | 90982371- | 90994673- | | |
| | | | | 91020447 | 90994692 | | |
| 489 | 84146 | ZNF644 | intron_04 | chr1: | chr1: | AACUAUGCAGUGUAGGCAGC | + |
| | | | | 90982371- | 90994654- | | |
| | | | | 91020447 | 90994673 | | |
| 490 | 84146 | ZNF644 | intron_04 | chr1: | chr1: | AGGGGUGAAACAAAAAUUCU | + |
| | | | | 90982371- | 90994468- | | |
| | | | | 91020447 | 90994487 | | |
| 491 | 84146 | ZNF644 | intron_04 | chr1: | chr1: | CACUGUAGGGCUCUGUAUUC | + |
| | | | | 90982371- | 90994390- | | |
| | | | | 91020447 | 90994409 | | |
| 492 | 84146 | ZNF644 | intron_04 | chr1: | chr1: | GUUCAGGGAUGAAAUUACUA | + |
| | | | | 90982371- | 90994219- | | |
| | | | | 91020447 | 90994238 | | |
| 493 | 84146 | ZNF644 | intron_04 | chr1: | chr1: | UCAUGGAGAGAACAUGUUCA | + |
| | | | | 90982371- | 90994204- | | |
| | | | | 91020447 | 90994223 | | |
| 494 | 84146 | ZNF644 | intron_04 | chr1: | chr1: | GCUAUCUUUGCCACACUUCA | + |
| | | | | 90982371- | 90994187- | | |
| | | | | 91020447 | 90994206 | | |
| 495 | 84146 | ZNF644 | intron_04 | chr1: | chr1: | AUGUAGGUUAGAGCAGCCAG | + |
| | | | | 90982371- | 90994165- | | |
| | | | | 91020447 | 90994184 | | |
| 496 | 84146 | ZNF644 | intron_04 | chr1: | chr1: | AACAGUUAUGAGAACGAUGU | + |
| | | | | 90982371- | 90994149- | | |
| | | | | 91020447 | 90994168 | | |
| 497 | 84146 | ZNF644 | intron_04 | chr1: | chr1: | AGUGUCACUUGAAUCCUCAG | + |
| | | | | 90982371- | 90994125- | | |
| | | | | 91020447 | 90994144 | | |
| 498 | 84146 | ZNF644 | intron_04 | chr1: | chr1: | GUUCAUGGAUGGGACUAAAU | + |
| | | | | 90982371- | 90994058- | | |
| | | | | 91020447 | 90994077 | | |
| 499 | 84146 | ZNF644 | intron_04 | chr1: | chr1: | UGUUCAUGGAUGGGACUAAA | + |
| | | | | 90982371- | 90994057- | | |
| | | | | 91020447 | 90994076 | | |
| 500 | 84146 | ZNF644 | intron_04 | chr1: | chr1: | AUCCUAGGCACCUGAAUGGA | + |
| | | | | 90982371- | 90993978- | | |
| | | | | 91020447 | 90993997 | | |
| 501 | 84146 | ZNF644 | intron_04 | chr1: | chr1: | CCCAAUCCUAGGCACCUGAA | + |
| | | | | 90982371- | 90993974- | | |
| | | | | 91020447 | 90993993 | | |
| 502 | 84146 | ZNF644 | intron_04 | chr1: | chr1: | AGUAACCUUAAUUUCCAGAU | + |
| | | | | 90982371- | 90993921- | | |
| | | | | 91020447 | 90993940 | | |
| 503 | 84146 | ZNF644 | intron_04 | chr1: | chr1: | GUUACUAUUAGUAAAAAUAG | − |
| | | | | 90982371- | 90993907- | | |
| | | | | 91020447 | 90993926 | | |
| 504 | 84146 | ZNF644 | intron_04 | chr1: | chr1: | UUACUGGAUCCAGCAAUACA | + |
| | | | | 90982371- | 90993815- | | |
| | | | | 91020447 | 90993834 | | |
| 505 | 84146 | ZNF644 | intron_04 | chr1: | chr1: | UUGUUCCUAGACAACUUUAC | + |
| | | | | 90982371- | 90993799- | | |
| | | | | 91020447 | 90993818 | | |
| 506 | 84146 | ZNF644 | intron_04 | chr1: | chr1: | UGGAUCCAGUAAAGUUGUCU | − |
| | | | | 90982371- | 90993807- | | |
| | | | | 91020447 | 90993826 | | |
| 507 | 84146 | ZNF644 | intron_04 | chr1: | chr1: | CUCUUAACUUUUGAAGACAA | + |
| | | | | 90982371- | 90993624- | | |
| | | | | 91020447 | 90993643 | | |
| 508 | 84146 | ZNF644 | intron_04 | chr1: | chr1: | UCAAUAUAAAAUAACUCCUA | − |
| | | | | 90982371- | 90993590- | | |
| | | | | 91020447 | 90993609 | | |
| 509 | 84146 | ZNF644 | intron_04 | chr1: | chr1: | UCAAGACCAGAAACGCCACC | + |
| | | | | 90982371- | 90993430- | | |
| | | | | 91020447 | 90993449 | | |
| 510 | 84146 | ZNF644 | intron_04 | chr1: | chr1: | AACUACAGCCAGCUCAAUAU | + |
| | | | | 90982371- | 90993290- | | |
| | | | | 91020447 | 90993309 | | |
| 511 | 84146 | ZNF644 | intron_04 | chr1: | chr1: | ACUCUCAUCUGCUUCAGAAU | + |
| | | | | 90982371- | 90993232- | | |
| | | | | 91020447 | 90993251 | | |
| 512 | 84146 | ZNF644 | intron_04 | chr1: | chr1: | AACUCUCAUCUGCUUCAGAA | + |
| | | | | 90982371- | 90993231- | | |
| | | | | 91020447 | 90993250 | | |
| 513 | 84146 | ZNF644 | intron_04 | chr1: | chr1: | GACCUUAUAAGAAACACCAG | + |
| | | | | 90982371- | 90993104- | | |
| | | | | 91020447 | 90993123 | | |
| 514 | 84146 | ZNF644 | intron_04 | chr1: | chr1: | UCAUCAAGAGGUGGUCCGCC | + |
| | | | | 90982371- | 90992749- | | |
| | | | | 91020447 | 90992768 | | |
| 515 | 84146 | ZNF644 | intron_04 | chr1: | chr1: | CGUUAUCCAUGUAUUUGGCA | + |
| | | | | 90982371- | 90992709- | | |
| | | | | 91020447 | 90992728 | | |
| 516 | 84146 | ZNF644 | intron_04 | chr1: | chr1: | CACCUCGUUAUCCAUGUAUU | + |
| | | | | 90982371- | 90992704- | | |
| | | | | 91020447 | 90992723 | | |
| 517 | 84146 | ZNF644 | intron_04 | chr1: | chr1: | AUUGUUUGCAUAUAGAAAUU | − |
| | | | | 90982371- | 90992587- | | |
| | | | | 91020447 | 90992606 | | |
| 518 | 84146 | ZNF644 | intron_04 | chr1: | chr1: | CAAAGAGUAAUCUGGACUAC | + |
| | | | | 90982371- | 90992532- | | |
| | | | | 91020447 | 90992551 | | |
| 519 | 84146 | ZNF644 | intron_04 | chr1: | chr1: | AAAUAUAGCAAAGAGUAAUC | + |
| | | | | 90982371- | 90992524- | | |
| | | | | 91020447 | 90992543 | | |
| 520 | 84146 | ZNF644 | intron_04 | chr1: | chr1: | CUAUAUUUGUUUCAUACAUC | − |
| | | | | 90982371- | 90992512- | | |
| | | | | 91020447 | 90992531 | | |
| 521 | 84146 | ZNF644 | intron_04 | chr1: | chr1: | UACCAACAUCUACCUACAAC | + |
| | | | | 90982371- | 90992446- | | |
| | | | | 91020447 | 90992465 | | |
| 522 | 84146 | ZNF644 | intron_04 | chr1: | chr1: | UACCAGUUGUAGGUAGAUGU | − |
| | | | | 90982371- | 90992451- | | |
| | | | | 91020447 | 90992470 | | |
| 523 | 84146 | ZNF644 | intron_04 | chr1: | chr1: | ACACCUGCAACAGAAUAUGU | + |
| | | | | 90982371- | 90991951- | | |
| | | | | 91020447 | 90991970 | | |
| 524 | 84146 | ZNF644 | intron_04 | chr1: | chr1: | GCGGAAAAAUGUCCACCAAC | + |
| | | | | 90982371- | 90991912- | | |
| | | | | 91020447 | 90991931 | | |
| 525 | 84146 | ZNF644 | intron_04 | chr1: | chr1: | GUCAAACAUACACCUACUAC | + |
| | | | | 90982371- | 90991381- | | |
| | | | | 91020447 | 90991400 | | |
| 526 | 84146 | ZNF644 | intron_04 | chr1: | chr1: | UGGCACCAGGAGAACUUUUG | + |
| | | | | 90982371- | 90990930- | | |
| | | | | 91020447 | 90990949 | | |
| 527 | 84146 | ZNF644 | intron_04 | chr1: | chr1: | CAGACCCCCAAAAAGGAACG | + |
| | | | | 90982371- | 90990889- | | |
| | | | | 91020447 | 90990908 | | |
| 528 | 84146 | ZNF644 | intron_04 | chr1: | chr1: | AAAGAGACAGACCCCCAAAA | + |
| | | | | 90982371- | 90990882- | | |
| | | | | 91020447 | 90990901 | | |
| 529 | 84146 | ZNF644 | intron_04 | chr1: | chr1: | AAAAGGAGUCUUGCCUCAGU | + |
| | | | | 90982371- | 90990828- | | |
| | | | | 91020447 | 90990847 | | |
| 530 | 84146 | ZNF644 | intron_04 | chr1: | chr1: | CACGUUAACACCAGACAAAA | + |
| | | | | 90982371- | 90990811- | | |
| | | | | 91020447 | 90990830 | | |
| 531 | 84146 | ZNF644 | intron_04 | chr1: | chr1: | AGUACCUGUUCUCUUAAGAG | + |
| | | | | 90982371- | 90990774- | | |
| | | | | 91020447 | 90990793 | | |
| 532 | 84146 | ZNF644 | intron_04 | chr1: | chr1: | GCAAGGAAGAGUCACCCAAA | + |
| | | | | 90982371- | 90990711- | | |
| | | | | 91020447 | 90990730 | | |
| 533 | 84146 | ZNF644 | intron_04 | chr1: | chr1: | AAAGCCUAAAGAAGUCAUGA | + |
| | | | | 90982371- | 90990596- | | |
| | | | | 91020447 | 90990615 | | |
| 534 | 84146 | ZNF644 | intron_04 | chr1: | chr1: | AAAAGCCUAAAGAAGUCAUG | + |
| | | | | 90982371- | 90990595- | | |
| | | | | 91020447 | 90990614 | | |
| 535 | 84146 | ZNF644 | intron_04 | chr1: | chr1: | AAAGUACAGAUCCCUGCCCA | + |
| | | | | 90982371- | 90990517- | | |
| | | | | 91020447 | 90990536 | | |
| 536 | 84146 | ZNF644 | intron_04 | chr1: | chr1: | AGGUGAGCAAACUUACUGCC | + |
| | | | | 90982371- | 90990492- | | |
| | | | | 91020447 | 90990511 | | |
| 537 | 84146 | ZNF644 | intron_04 | chr1: | chr1: | UGAACCCUGAAAGGGAAACA | + |
| | | | | 90982371- | 90990472- | | |
| | | | | 91020447 | 90990491 | | |
| 538 | 84146 | ZNF644 | intron_04 | chr1: | chr1: | CCUGAUGGUGAACCCUGAAA | + |
| | | | | 90982371- | 90990464- | | |
| | | | | 91020447 | 90990483 | | |
| 539 | 84146 | ZNF644 | intron_04 | chr1: | chr1: | CCCUGAUGGUGAACCCUGAA | + |
| | | | | 90982371- | 90990463- | | |
| | | | | 91020447 | 90990482 | | |
| 540 | 84146 | ZNF644 | intron_04 | chr1: | chr1: | AGUACAUAUAAGAGCCAAGG | + |
| | | | | 90982371- | 90990346- | | |
| | | | | 91020447 | 90990365 | | |
| 541 | 84146 | ZNF644 | intron_04 | chr1: | chr1: | GAAAGUACAUAUAAGAGCCA | + |
| | | | | 90982371- | 90990343- | | |
| | | | | 91020447 | 90990362 | | |
| 542 | 84146 | ZNF644 | intron_04 | chr1: | chr1: | GGAGUGGUUGUCAAAAUCUG | + |
| | | | | 90982371- | 90990132- | | |
| | | | | 91020447 | 90990151 | | |
| 543 | 84146 | ZNF644 | intron_04 | chr1: | chr1: | GAACCCUUGGUGCACUGUUG | + |
| | | | | 90982371- | 90989697- | | |
| | | | | 91020447 | 90989716 | | |
| 544 | 84146 | ZNF644 | intron_04 | chr1: | chr1: | AGGGGAAGGCUUCGGGACAC | + |
| | | | | 90982371- | 90988945- | | |
| | | | | 91020447 | 90988964 | | |
| 545 | 84146 | ZNF644 | intron_04 | chr1: | chr1: | GGGGCCAAUACUAUACAGAG | + |
| | | | | 90982371- | 90988764- | | |
| | | | | 91020447 | 90988783 | | |
| 546 | 84146 | ZNF644 | intron_04 | chr1: | chr1: | AACACACUGUACCCACAGAU | + |
| | | | | 90982371- | 90988410- | | |
| | | | | 91020447 | 90988429 | | |
| 547 | 84146 | ZNF644 | intron_04 | chr1: | chr1: | ACUUACUCUUACCCAACCAA | + |
| | | | | 90982371- | 90986909- | | |
| | | | | 91020447 | 90986928 | | |
| 548 | 84146 | ZNF644 | intron_04 | chr1: | chr1: | UUAAAAGCAGUUGGCACCGC | + |
| | | | | 90982371- | 90986118- | | |
| | | | | 91020447 | 90986137 | | |
| 549 | 84146 | ZNF644 | intron_04 | chr1: | chr1: | UUACUUAUAUAAGUGGGAGG | + |
| | | | | 90982371- | 90986074- | | |
| | | | | 91020447 | 90986093 | | |
| 550 | 84146 | ZNF644 | intron_04 | chr1: | chr1: | AUUACUUAUAUAAGUGGGAG | + |
| | | | | 90982371- | 90986073- | | |
| | | | | 91020447 | 90986092 | | |
| 551 | 84146 | ZNF644 | intron_04 | chr1: | chr1: | UAUUACUUAUAUAAGUGGGA | + |
| | | | | 90982371- | 90986072- | | |
| | | | | 91020447 | 90986091 | | |
| 552 | 84146 | ZNF644 | intron_04 | chr1: | chr1: | AUAUUACUUAUAUAAGUGGG | + |
| | | | | 90982371- | 90986071- | | |
| | | | | 91020447 | 90986090 | | |
| 553 | 84146 | ZNF644 | intron_04 | chr1: | chr1: | GGAAUAUUACUUAUAUAAGU | + |
| | | | | 90982371- | 90986068- | | |
| | | | | 91020447 | 90986087 | | |
| 554 | 84146 | ZNF644 | intron_04 | chr1: | chr1: | UGGAAUAUUACUUAUAUAAG | + |
| | | | | 90982371- | 90986067- | | |
| | | | | 91020447 | 90986086 | | |
| 555 | 84146 | ZNF644 | intron_04 | chr1: | chr1: | CAUUCCUAACAAGGUACACA | + |
| | | | | 90982371- | 90985907- | | |
| | | | | 91020447 | 90985926 | | |
| 556 | 84146 | ZNF644 | intron_04 | chr1: | chr1: | UGGAGAGUACGAGCAAAGCA | − |
| | | | | 90982371- | 90985070- | | |
| | | | | 91020447 | 90985089 | | |
| 557 | 84146 | ZNF644 | intron_04 | chr1: | chr1: | CUGCAAAAGAGAGAUGCCUC | + |
| | | | | 90982371- | 90984747- | | |
| | | | | 91020447 | 90984766 | | |
| 558 | 84146 | ZNF644 | intron_04 | chr1: | chr1: | UACAAGGUAUGCACACACAG | + |
| | | | | 90982371- | 90984715- | | |
| | | | | 91020447 | 90984734 | | |
| 559 | 84146 | ZNF644 | intron_04 | chr1: | chr1: | UUCAGGAAUCAAGUUAAAUA | + |
| | | | | 90982371- | 90984377- | | |
| | | | | 91020447 | 90984396 | | |
| 560 | 84146 | ZNF644 | intron_04 | chr1: | chr1: | UAUUAGAGCGAGAGCCUUUC | + |
| | | | | 90982371- | 90984360- | | |
| | | | | 91020447 | 90984379 | | |
| 561 | 84146 | ZNF644 | intron_04 | chr1: | chr1: | ACGAACUGCAUUGGUCUGCU | + |
| | | | | 90982371- | 90984289- | | |
| | | | | 91020447 | 90984308 | | |
| 562 | 84146 | ZNF644 | intron_04 | chr1: | chr1: | AAAGAUGUUACGAACUGCAU | + |
| | | | | 90982371- | 90984280- | | |
| | | | | 91020447 | 90984299 | | |
| 563 | 84146 | ZNF644 | intron_04 | chr1: | chr1: | GUUCGUAACAUCUUUUAUAU | − |
| | | | | 90982371- | 90984275- | | |
| | | | | 91020447 | 90984294 | | |
| 564 | 84146 | ZNF644 | intron_04 | chr1: | chr1: | UUCGUAACAUCUUUUAUAUA | − |
| | | | | 90982371- | 90984274- | | |
| | | | | 91020447 | 90984293 | | |
| 565 | 84146 | ZNF644 | intron_04 | chr1: | chr1: | UCGUAACAUCUUUUAUAUAG | − |
| | | | | 90982371- | 90984273- | | |
| | | | | 91020447 | 90984292 | | |
| 566 | 84146 | ZNF644 | intron_04 | chr1: | chr1: | CUGAAGACAGCAAGUUCUCA | + |
| | | | | 90982371- | 90984231- | | |
| | | | | 91020447 | 90984250 | | |
| 567 | 84146 | ZNF644 | intron_04 | chr1: | chr1: | GAAGGACAUGGGCCAUAGCC | + |
| | | | | 90982371- | 90983991- | | |
| | | | | 91020447 | 90984010 | | |
| 568 | 84146 | ZNF644 | intron_04 | chr1: | chr1: | UUUCGUAGACUGGAAGGACA | + |
| | | | | 90982371- | 90983979- | | |
| | | | | 91020447 | 90983998 | | |
| 569 | 84146 | ZNF644 | intron_04 | chr1: | chr1: | UGCUGCUUUCGUAGACUGGA | + |
| | | | | 90982371- | 90983973- | | |
| | | | | 91020447 | 90983992 | | |
| 570 | 84146 | ZNF644 | intron_04 | chr1: | chr1: | AAAAUGCUGCUUUCGUAGAC | + |
| | | | | 90982371- | 90983969- | | |
| | | | | 91020447 | 90983988 | | |
| 571 | 84146 | ZNF644 | intron_04 | chr1: | chr1: | AGAAGACAAGCGGAGAGGCC | + |
| | | | | 90982371- | 90983629- | | |
| | | | | 91020447 | 90983648 | | |
| 572 | 84146 | ZNF644 | intron_04 | chr1: | chr1: | GAGAAGACAAGCGGAGAGGC | + |
| | | | | 90982371- | 90983628- | | |
| | | | | 91020447 | 90983647 | | |
| 573 | 84146 | ZNF644 | intron_04 | chr1: | chr1: | GUCUAUUAUAAACGACUGAG | + |
| | | | | 90982371- | 90983383- | | |
| | | | | 91020447 | 90983402 | | |
| 574 | 84146 | ZNF644 | intron_04 | chr1: | chr1: | CAACCCAGAAGUGGACAUGG | + |
| | | | | 90982371- | 90983316- | | |
| | | | | 91020447 | 90983335 | | |
| 575 | 84146 | ZNF644 | intron_04 | chr1: | chr1: | GAUACGAUGCAGAAAAUUCU | − |
| | | | | 90982371- | 90983247- | | |
| | | | | 91020447 | 90983266 | | |
| 576 | 84146 | ZNF644 | intron_04 | chr1: | chr1: | CUAGGUCUGUGGUUCUAGGA | − |
| | | | | 90982371- | 90983229- | | |
| | | | | 91020447 | 90983248 | | |
| 577 | 84146 | ZNF644 | intron_04 | chr1: | chr1: | CCUACCUCUGUUCUCAAGCU | + |
| | | | | 90982371- | 90983160- | | |
| | | | | 91020447 | 90983179 | | |
| 578 | 84146 | ZNF644 | intron_04 | chr1: | chr1: | GUAACCUAGCUUGAGAACAG | − |
| | | | | 90982371- | 90983167- | | |
| | | | | 91020447 | 90983186 | | |
| 579 | 84146 | ZNF644 | intron_04 | chr1: | chr1: | CCUAGCUUGAGAACAGAGGU | − |
| | | | | 90982371- | 90983163- | | |
| | | | | 91020447 | 90983182 | | |
| 580 | 84146 | ZNF644 | intron_04 | chr1: | chr1: | UCACAAGCUAGUUUCAGGUA | − |
| | | | | 90982371- | 90983098- | | |
| | | | | 91020447 | 90983117 | | |
| 581 | 84146 | ZNF644 | intron_04 | chr1: | chr1: | CACAAGCUAGUUUCAGGUAU | − |
| | | | | 90982371- | 90983097- | | |
| | | | | 91020447 | 90983116 | | |
| 582 | 84146 | ZNF644 | intron_04 | chr1: | chr1: | ACAAGCUAGUUUCAGGUAUG | − |
| | | | | 90982371- | 90983096- | | |
| | | | | 91020447 | 90983115 | | |
| 583 | 84146 | ZNF644 | intron_04 | chr1: | chr1: | UAUGGUAAAAUUAGUAAUGG | − |
| | | | | 90982371- | 90983021- | | |
| | | | | 91020447 | 90983040 | | |
| 584 | 84146 | ZNF644 | intron_04 | chr1: | chr1: | UUCUCUAGAAUAAUGAUGCU | |
| | | | | 90982371- | 90982896- | | |
| | | | | 91020447 | 90982915 | | |
| 585 | 84146 | ZNF644 | intron_04 | chr1: | chr1: | AUUUAGUAAGUAAUCAGUGU | − |
| | | | | 90982371- | 90982872- | | |
| | | | | 91020447 | 90982891 | | |
| 586 | 84146 | ZNF644 | intron_04 | chr1: | chr1: | UCAAAAAAAGCAGUGAGGUU | − |
| | | | | 90982371- | 90982561- | | |
| | | | | 91020447 | 90982580 | | |
| 587 | 84146 | ZNF644 | intron_04 | chr1: | chr1: | AUAAUAGUGUAUUGUAGCUA | − |
| | | | | 90982371- | 90982430- | | |
| | | | | 91020447 | 90982449 | | |
| 588 | 84146 | ZNF644 | exon_05_c.2/ | chr1: | chr1: | AUGUUAAUAAGACAAAAUCU | − |
| | | | nc.3 | 90982310- | 90982312- | | |
| | | | | 90982331 | 90982331 | | |
| 589 | 84146 | ZNF644 | intron_05 | chr1: | chr1: | AAUUGACUUUUUGACACACU | − |
| | | | | 90973214- | 90982163- | | |
| | | | | 90982309 | 90982182 | | |
| 590 | 84146 | ZNF644 | intron_05 | chr1: | chr1: | ACUUGGUCCAGUUGAUUUGA | − |
| | | | | 90973214- | 90982146- | | |
| | | | | 90982309 | 90982165 | | |
| 591 | 84146 | ZNF644 | intron_05 | chr1: | chr1: | CUUGGUCCAGUUGAUUUGAU | − |
| | | | | 90973214- | 90982145- | | |
| | | | | 90982309 | 90982164 | | |
| 592 | 84146 | ZNF644 | intron_05 | chr1: | chr1: | GUUAACCUCAACACAAAAGA | + |
| | | | | 90973214- | 90982075- | | |
| | | | | 90982309 | 90982094 | | |
| 593 | 84146 | ZNF644 | intron_05 | chr1: | chr1: | UGGUCAGAGUUCAAUAAGAG | + |
| | | | | 90973214- | 90981987- | | |
| | | | | 90982309 | 90982006 | | |
| 594 | 84146 | ZNF644 | intron_05 | chr1: | chr1: | UUCAUAUACUUUAUCAACAA | + |
| | | | | 90973214- | 90981925- | | |
| | | | | 90982309 | 90981944 | | |
| 595 | 84146 | ZNF644 | intron_05 | chr1: | chr1: | AUUCAGUAGAGACACCCACU | + |
| | | | | 90973214- | 90981766- | | |
| | | | | 90982309 | 90981785 | | |
| 596 | 84146 | ZNF644 | intron_05 | chr1: | chr1: | UGAAUUGGCAUCAUGUACUU | − |
| | | | | 90973214- | 90981689- | | |
| | | | | 90982309 | 90981708 | | |
| 597 | 84146 | ZNF644 | intron_05 | chr1: | chr1: | AGCUGAAACAUCUGGUUAGA | + |
| | | | | 90973214- | 90981520- | | |
| | | | | 90982309 | 90981539 | | |
| 598 | 84146 | ZNF644 | intron_05 | chr1: | chr1: | GAAAUCUCAGUCAGUGGUUU | − |
| | | | | 90973214- | 90981414- | | |
| | | | | 90982309 | 90981433 | | |
| 599 | 84146 | ZNF644 | intron_05 | chr1: | chr1: | AGGUACUAUAACGAUCAAUA | + |
| | | | | 90973214- | 90981280- | | |
| | | | | 90982309 | 90981299 | | |
| 600 | 84146 | ZNF644 | intron_05 | chr1: | chr1: | AAAUUUUUCUACACAACAAG | + |
| | | | | 90973214- | 90981257- | | |
| | | | | 90982309 | 90981276 | | |
| 601 | 84146 | ZNF644 | intron_05 | chr1: | chr1: | UCCACUUUAAGAAGAUACAU | + |
| | | | | 90973214- | 90981155- | | |
| | | | | 90982309 | 90981174 | | |
| 602 | 84146 | ZNF644 | intron_05 | chr1: | chr1: | UGUACUUGAAGGUCUUAUUA | − |
| | | | | 90973214- | 90981119- | | |
| | | | | 90982309 | 90981138 | | |
| 603 | 84146 | ZNF644 | intron_05 | chr1: | chr1: | UAUUGCUUGUGGGAUGACAC | − |
| | | | | 90973214- | 90981034- | | |
| | | | | 90982309 | 90981053 | | |
| 604 | 84146 | ZNF644 | intron_05 | chr1: | chr1: | AGGCUAUUAGACUGGAAGCG | + |
| | | | | 90973214- | 90980805- | | |
| | | | | 90982309 | 90980824 | | |
| 605 | 84146 | ZNF644 | intron_05 | chr1: | chr1: | AAAGGCUAUUAGACUGGAAG | + |
| | | | | 90973214- | 90980803- | | |
| | | | | 90982309 | 90980822 | | |
| 606 | 84146 | ZNF644 | intron_05 | chr1: | chr1: | AGGGGAAAAGGCUAUUAGAC | + |
| | | | | 90973214- | 90980797- | | |
| | | | | 90982309 | 90980816 | | |
| 607 | 84146 | ZNF644 | intron_05 | chr1: | chr1: | CAGAAAGAUGAUUACUUCAG | + |
| | | | | 90973214- | 90980779- | | |
| | | | | 90982309 | 90980798 | | |
| 608 | 84146 | ZNF644 | intron_05 | chr1: | chr1: | UAACUGCAUGCCGUGUUAUA | + |
| | | | | 90973214- | 90980428- | | |
| | | | | 90982309 | 90980447 | | |
| 609 | 84146 | ZNF644 | intron_05 | chr1: | chr1: | UCAUAUAAUCCUCAGCAUUC | + |
| | | | | 90973214- | 90980389- | | |
| | | | | 90982309 | 90980408 | | |
| 610 | 84146 | ZNF644 | intron_05 | chr1: | chr1: | GUAGCUAUGCCAGAAUGCUG | − |
| | | | | 90973214- | 90980401- | | |
| | | | | 90982309 | 90980420 | | |
| 611 | 84146 | ZNF644 | intron_05 | chr1: | chr1: | GAGGUCUCUGGCAACUGGGG | + |
| | | | | 90973214- | 90980292- | | |
| | | | | 90982309 | 90980311 | | |
| 612 | 84146 | ZNF644 | intron_05 | chr1: | chr1: | AAAGAGGUCUCUGGCAACUG | + |
| | | | | 90973214- | 90980289- | | |
| | | | | 90982309 | 90980308 | | |
| 613 | 84146 | ZNF644 | intron_05 | chr1: | chr1: | GCAAAGAGGUCUCUGGCAAC | + |
| | | | | 90973214- | 90980287- | | |
| | | | | 90982309 | 90980306 | | |
| 614 | 84146 | ZNF644 | intron_05 | chr1: | chr1: | GAUCAGAUGAUGCUUCAGAU | + |
| | | | | 90973214- | 90980237- | | |
| | | | | 90982309 | 90980256 | | |
| 615 | 84146 | ZNF644 | intron_05 | chr1: | chr1: | UACGGCCAGGCAUGGCUUAA | + |
| | | | | 90973214- | 90980215- | | |
| | | | | 90982309 | 90980234 | | |
| 616 | 84146 | ZNF644 | intron_05 | chr1: | chr1: | CUGAUCCUUUAAGCCAUGCC | − |
| | | | | 90973214- | 90980223- | | |
| | | | | 90982309 | 90980242 | | |
| 617 | 84146 | ZNF644 | intron_05 | chr1: | chr1: | UAAGAUAAACGAGACAUGGA | + |
| | | | | 90973214- | 90980152- | | |
| | | | | 90982309 | 90980171 | | |
| 618 | 84146 | ZNF644 | intron_05 | chr1: | chr1: | CCUGGGAGAACUCUUUUCAU | + |
| | | | | 90973214- | 90980076- | | |
| | | | | 90982309 | 90980095 | | |
| 619 | 84146 | ZNF644 | intron_05 | chr1: | chr1: | CCAAUGAAAAGAGUUCUCCC | − |
| | | | | 90973214- | 90980079- | | |
| | | | | 90982309 | 90980098 | | |
| 620 | 84146 | ZNF644 | intron_05 | chr1: | chr1: | UUGAGUUGCUGUUGGGCCAU | − |
| | | | | 90973214- | 90980007- | | |
| | | | | 90982309 | 90980026 | | |
| 621 | 84146 | ZNF644 | intron_05 | chr1: | chr1: | GUUGCUGUUGGGCCAUAGGA | − |
| | | | | 90973214- | 90980003- | | |
| | | | | 90982309 | 90980022 | | |
| 622 | 84146 | ZNF644 | intron_05 | chr1: | chr1: | AAUGUCUAUAAUGCUGUCUG | + |
| | | | | 90973214- | 90979961- | | |
| | | | | 90982309 | 90979980 | | |
| 623 | 84146 | ZNF644 | intron_05 | chr1: | chr1: | ACUAGCCACUGUAUUUCAAC | + |
| | | | | 90973214- | 90979897- | | |
| | | | | 90982309 | 90979916 | | |
| 624 | 84146 | ZNF644 | intron_05 | chr1: | chr1: | UCAAUCCAGUUGAAAUACAG | − |
| | | | | 90973214- | 90979905- | | |
| | | | | 90982309 | 90979924 | | |
| 625 | 84146 | ZNF644 | intron_05 | chr1: | chr1: | AACAAUGACAGUCUUACUUU | + |
| | | | | 90973214- | 90979855- | | |
| | | | | 90982309 | 90979874 | | |
| 626 | 84146 | ZNF644 | intron_05 | chr1: | chr1: | UGAUUAGUAUUUCCAAGUGG | − |
| | | | | 90973214- | 90979830- | | |
| | | | | 90982309 | 90979849 | | |
| 627 | 84146 | ZNF644 | intron_05 | chr1: | chr1: | CAUGGAUUAAUACCUCCACU | + |
| | | | | 90973214- | 90979815- | | |
| | | | | 90982309 | 90979834 | | |
| 628 | 84146 | ZNF644 | intron_05 | chr1: | chr1: | CACUUCAAAUUUCUGCAACA | + |
| | | | | 90973214- | 90979797- | | |
| | | | | 90982309 | 90979816 | | |
| 629 | 84146 | ZNF644 | intron_05 | chr1: | chr1: | GACUAAUACAAGUAGAAGAU | − |
| | | | | 90973214- | 90979710- | | |
| | | | | 90982309 | 90979729 | | |
| 630 | 84146 | ZNF644 | intron_05 | chr1: | chr1: | GAAAUUGUAGAAUCAUAGCC | − |
| | | | | 90973214- | 90979607- | | |
| | | | | 90982309 | 90979626 | | |
| 631 | 84146 | ZNF644 | intron_05 | chr1: | chr1: | UAGCAAGCUUCCUAAGCUGU | + |
| | | | | 90973214- | 90979559- | | |
| | | | | 90982309 | 90979578 | | |
| 632 | 84146 | ZNF644 | intron_05 | chr1: | chr1: | GUGUUUGAAAUUUGGCUAGU | − |
| | | | | 90973214- | 90979093- | | |
| | | | | 90982309 | 90979112 | | |
| 633 | 84146 | ZNF644 | intron_05 | chr1: | chr1: | UGUUUGAAAUUUGGCUAGUU | − |
| | | | | 90973214- | 90979092- | | |
| | | | | 90982309 | 90979111 | | |
| 634 | 84146 | ZNF644 | intron_05 | chr1: | chr1: | UGAAAUUUGGCUAGUUGGGU | − |
| | | | | 90973214- | 90979088- | | |
| | | | | 90982309 | 90979107 | | |
| 635 | 84146 | ZNF644 | intron_05 | chr1: | chr1: | GGUUGGCUAUAGUAAUCAUC | − |
| | | | | 90973214- | 90979071- | | |
| | | | | 90982309 | 90979090 | | |
| 636 | 84146 | ZNF644 | intron_05 | chr1: | chr1: | GGAAGAACCUCUUCUUGCUU | + |
| | | | | 90973214- | 90979006- | | |
| | | | | 90982309 | 90979025 | | |
| 637 | 84146 | ZNF644 | intron_05 | chr1: | chr1: | GCAACAAAUUGAUCCCUGUG | + |
| | | | | 90973214- | 90978985- | | |
| | | | | 90982309 | 90979004 | | |
| 638 | 84146 | ZNF644 | intron_05 | chr1: | chr1: | UAUGUCUAAGAAAACCCUUG | + |
| | | | | 90973214- | 90978880- | | |
| | | | | 90982309 | 90978899 | | |
| 639 | 84146 | ZNF644 | intron_05 | chr1: | chr1: | AAAGAACCAAGGUCUAUAUA | + |
| | | | | 90973214- | 90978849- | | |
| | | | | 90982309 | 90978868 | | |
| 640 | 84146 | ZNF644 | intron_05 | chr1: | chr1: | CAUGUUGUUGGUUAGAGUUG | − |
| | | | | 90973214- | 90978790- | | |
| | | | | 90982309 | 90978809 | | |
| 641 | 84146 | ZNF644 | intron_05 | chr1: | chr1: | AUCUCAAGGUUAAAUUUCUC | + |
| | | | | 90973214- | 90978762- | | |
| | | | | 90982309 | 90978781 | | |
| 642 | 84146 | ZNF644 | intron_05 | chr1: | chr1: | AUGUAAGUUAGGCAAUCUCA | + |
| | | | | 90973214- | 90978748- | | |
| | | | | 90982309 | 90978767 | | |
| 643 | 84146 | ZNF644 | intron_05 | chr1: | chr1: | AUUUCAAAUCCUUGGAUUCG | + |
| | | | | 90973214- | 90978597- | | |
| | | | | 90982309 | 90978616 | | |
| 644 | 84146 | ZNF644 | intron_05 | chr1: | chr1: | AAUUUCAAAUCCUUGGAUUC | + |
| | | | | 90973214- | 90978596- | | |
| | | | | 90982309 | 90978615 | | |
| 645 | 84146 | ZNF644 | intron_05 | chr1: | chr1: | CUGCAGAAUCCCCGAAUCCA | − |
| | | | | 90973214- | 90978609- | | |
| | | | | 90982309 | 90978628 | | |
| 646 | 84146 | ZNF644 | intron_05 | chr1: | chr1: | UACAAUCUCAUCUAGAAUGA | + |
| | | | | 90973214- | 90978430- | | |
| | | | | 90982309 | 90978449 | | |
| 647 | 84146 | ZNF644 | intron_05 | chr1: | chr1: | CUUGGCAAUACAGCUGGAAA | + |
| | | | | 90973214- | 90978394- | | |
| | | | | 90982309 | 90978413 | | |
| 648 | 84146 | ZNF644 | intron_05 | chr1: | chr1: | ACUUGGCAAUACAGCUGGAA | + |
| | | | | 90973214- | 90978393- | | |
| | | | | 90982309 | 90978412 | | |
| 649 | 84146 | ZNF644 | intron_05 | chr1: | chr1: | CUGUUACUUGGCAAUACAGC | + |
| | | | | 90973214- | 90978388- | | |
| | | | | 90982309 | 90978407 | | |
| 650 | 84146 | ZNF644 | intron_05 | chr1: | chr1: | ACUAAAUACUAACUGUUACU | + |
| | | | | 90973214- | 90978376- | | |
| | | | | 90982309 | 90978395 | | |
| 651 | 84146 | ZNF644 | intron_05 | chr1: | chr1: | UGCUGUACUAUUUGCUCAUA | + |
| | | | | 90973214- | 90977999- | | |
| | | | | 90982309 | 90978018 | | |
| 652 | 84146 | ZNF644 | intron_05 | chr1: | chr1: | UGGGGAGUAUAGUGCUGGGU | + |
| | | | | 90973214- | 90977945- | | |
| | | | | 90982309 | 90977964 | | |
| 653 | 84146 | ZNF644 | intron_05 | chr1: | chr1: | AUGGGGAGUAUAGUGCUGGG | + |
| | | | | 90973214- | 90977944- | | |
| | | | | 90982309 | 90977963 | | |
| 654 | 84146 | ZNF644 | intron_05 | chr1: | chr1: | CUUAUGGGGAGUAUAGUGCU | + |
| | | | | 90973214- | 90977941- | | |
| | | | | 90982309 | 90977960 | | |
| 655 | 84146 | ZNF644 | intron_05 | chr1: | chr1: | ACUUAUGGGGAGUAUAGUGC | + |
| | | | | 90973214- | 90977940- | | |
| | | | | 90982309 | 90977959 | | |
| 656 | 84146 | ZNF644 | intron_05 | chr1: | chr1: | GCUAUUUUUGGUAACUUAUG | + |
| | | | | 90973214- | 90977927- | | |
| | | | | 90982309 | 90977946 | | |
| 657 | 84146 | ZNF644 | intron_05 | chr1: | chr1: | AGCUAUUUUUGGUAACUUAU | + |
| | | | | 90973214- | 90977926- | | |
| | | | | 90982309 | 90977945 | | |
| 658 | 84146 | ZNF644 | intron_05 | chr1: | chr1: | UUGCAGCAGUACUUAUAUAC | + |
| | | | | 90973214- | 90977780- | | |
| | | | | 90982309 | 90977799 | | |
| 659 | 84146 | ZNF644 | intron_05 | chr1: | chr1: | CUAGCAAUAAGAGUAUUAAG | + |
| | | | | 90973214- | 90977632- | | |
| | | | | 90982309 | 90977651 | | |
| 660 | 84146 | ZNF644 | intron_05 | chr1: | chr1: | UUCCACCAAUUAGACUAGCA | + |
| | | | | 90973214- | 90977561- | | |
| | | | | 90982309 | 90977580 | | |
| 661 | 84146 | ZNF644 | intron_05 | chr1: | chr1: | UAAAUUGUGAGAGUGCUCAA | − |
| | | | | 90973214- | 90977513- | | |
| | | | | 90982309 | 90977532 | | |
| 662 | 84146 | ZNF644 | intron_05 | chr1: | chr1: | CAUUACAACUUAAGUUCUGU | − |
| | | | | 90973214- | 90977474- | | |
| | | | | 90982309 | 90977493 | | |
| 663 | 84146 | ZNF644 | intron_05 | chr1: | chr1: | AUGUUAAUUGAUGGAACUGA | + |
| | | | | 90973214- | 90977410- | | |
| | | | | 90982309 | 90977429 | | |
| 664 | 84146 | ZNF644 | intron_05 | chr1: | chr1: | GUAGCUUAUCAUACAUAAUA | + |
| | | | | 90973214- | 90977095- | | |
| | | | | 90982309 | 90977114 | | |
| 665 | 84146 | ZNF644 | intron_05 | chr1: | chr1: | GAGUUCACUCACAUUAAAGC | + |
| | | | | 90973214- | 90977055- | | |
| | | | | 90982309 | 90977074 | | |
| 666 | 84146 | ZNF644 | intron_05 | chr1: | chr1: | UUAACAGCACAGUGAAACCA | + |
| | | | | 90973214- | 90976704- | | |
| | | | | 90982309 | 90976723 | | |
| 667 | 84146 | ZNF644 | intron_05 | chr1: | chr1: | AGCAUGGCUGAAACUUUCUU | + |
| | | | | 90973214- | 90976623- | | |
| | | | | 90982309 | 90976642 | | |
| 668 | 84146 | ZNF644 | intron_05 | chr1: | chr1: | AUGGUUUCCUGUUCACAUAU | + |
| | | | | 90973214- | 90976524- | | |
| | | | | 90982309 | 90976543 | | |
| 669 | 84146 | ZNF644 | intron_05 | chr1: | chr1: | GGUCUGAAGGAGCAGUUUUA | + |
| | | | | 90973214- | 90976505- | | |
| | | | | 90982309 | 90976524 | | |
| 670 | 84146 | ZNF644 | intron_05 | chr1: | chr1: | AUAGCCAUGGCAAAAUUCUU | + |
| | | | | 90973214- | 90976484- | | |
| | | | | 90982309 | 90976503 | | |
| 671 | 84146 | ZNF644 | intron_05 | chr1: | chr1: | AGAAGGCUAAAGUAUAGCCA | + |
| | | | | 90973214- | 90976471- | | |
| | | | | 90982309 | 90976490 | | |
| 672 | 84146 | ZNF644 | intron_05 | chr1: | chr1: | CAGUUUAGACCAAUCAUAGA | + |
| | | | | 90973214- | 90976454- | | |
| | | | | 90982309 | 90976473 | | |
| 673 | 84146 | ZNF644 | intron_05 | chr1: | chr1: | UAUAACUAGUUGCAUGGUAC | + |
| | | | | 90973214- | 90976326- | | |
| | | | | 90982309 | 90976345 | | |
| 674 | 84146 | ZNF644 | intron_05 | chr1: | chr1: | UGCCAGUAACUCUGGCAAUA | + |
| | | | | 90973214- | 90976064- | | |
| | | | | 90982309 | 90976083 | | |
| 675 | 84146 | ZNF644 | intron_05 | chr1: | chr1: | UAAGAAACUGCCAGUAACUC | + |
| | | | | 90973214- | 90976056- | | |
| | | | | 90982309 | 90976075 | | |
| 676 | 84146 | ZNF644 | intron_05 | chr1: | chr1: | GUCCAUAUUGCCAGAGUUAC | − |
| | | | | 90973214- | 90976069- | | |
| | | | | 90982309 | 90976088 | | |
| 677 | 84146 | ZNF644 | intron_05 | chr1: | chr1: | CAAAAUAAUGAUGAGUAUGA | + |
| | | | | 90973214- | 90975899- | | |
| | | | | 90982309 | 90975918 | | |
| 678 | 84146 | ZNF644 | intron_05 | chr1: | chr1: | ACUUUUAGGGUCUUCUAACU | − |
| | | | | 90973214- | 90975404- | | |
| | | | | 90982309 | 90975423 | | |
| 679 | 84146 | ZNF644 | intron_05 | chr1: | chr1: | UCUAACUAGGCUGAAGCCCG | − |
| | | | | 90973214- | 90975391- | | |
| | | | | 90982309 | 90975410 | | |
| 680 | 84146 | ZNF644 | intron_05 | chr1: | chr1: | CUAACUAGGCUGAAGCCCGU | − |
| | | | | 90973214- | 90975390- | | |
| | | | | 90982309 | 90975409 | | |
| 681 | 84146 | ZNF644 | intron_05 | chr1: | chr1: | GAUCACACUUUACUAGUCUG | − |
| | | | | 90973214- | 90975289- | | |
| | | | | 90982309 | 90975308 | | |
| 682 | 84146 | ZNF644 | intron_05 | chr1: | chr1: | ACUUCUACCACUCUCAAGUC | + |
| | | | | 90973214- | 90974673- | | |
| | | | | 90982309 | 90974692 | | |
| 683 | 84146 | ZNF644 | intron_05 | chr1: | chr1: | UUACAAUUAACCACCAAAGC | + |
| | | | | 90973214- | 90974598- | | |
| | | | | 90982309 | 90974617 | | |
| 684 | 84146 | ZNF644 | intron_05 | chr1: | chr1: | UCACAUCUCGAGUUAUAGAU | + |
| | | | | 90973214- | 90974377- | | |
| | | | | 90982309 | 90974396 | | |
| 685 | 84146 | ZNF644 | intron_05 | chr1: | chr1: | UCCUAAAACUUUUAUGGGAU | + |
| | | | | 90973214- | 90974015- | | |
| | | | | 90982309 | 90974034 | | |
| 686 | 84146 | ZNF644 | intron_05 | chr1: | chr1: | UAAGUCAUUCAAUUUAUAGG | + |
| | | | | 90973214- | 90973789- | | |
| | | | | 90982309 | 90973808 | | |
| 687 | 84146 | ZNF644 | intron_05 | chr1: | chr1: | AAGAGCCCGUCAUCUAUAGA | + |
| | | | | 90973214- | 90973717- | | |
| | | | | 90982309 | 90973736 | | |
| 688 | 84146 | ZNF644 | intron_05 | chr1: | chr1: | GAAGAGCCCGUCAUCUAUAG | + |
| | | | | 90973214- | 90973716- | | |
| | | | | 90982309 | 90973735 | | |
| 689 | 84146 | ZNF644 | intron_05 | chr1: | chr1: | GCAGUGCCCUCUAUAGAUGA | − |
| | | | | 90973214- | 90973726- | | |
| | | | | 90982309 | 90973745 | | |
| 690 | 84146 | ZNF644 | intron_05 | chr1: | chr1: | CAGUGCCCUCUAUAGAUGAC | − |
| | | | | 90973214- | 90973725- | | |
| | | | | 90982309 | 90973744 | | |
| 691 | 84146 | ZNF644 | intron_05 | chr1: | chr1: | AAAUAAGUCUGCAUUUAUCA | + |
| | | | | 90973214- | 90973556- | | |
| | | | | 90982309 | 90973575 | | |
| 692 | 84146 | ZNF644 | intron_05 | chr1: | chr1: | AACUACCCCGUUCAAAUCCU | + |
| | | | | 90973214- | 90973418- | | |
| | | | | 90982309 | 90973437 | | |
| 693 | 84146 | ZNF644 | intron_05 | chr1: | chr1: | GUGAGAGUAUAAUACAUAAA | + |
| | | | | 90973214- | 90973235- | | |
| | | | | 90982309 | 90973254 | | |
| 694 | 84146 | ZNF644 | intron_05 | chr1: | chr1: | UCCAGAAUAACCUGAGAGGG | + |
| | | | | 90973214- | 90973203- | | |
| | | | | 90982309 | 90973222 | | |
| 695 | 84146 | ZNF644 | intron_05 | chr1: | chr1: | UUGCUCCAGAAUAACCUGAG | + |
| | | | | 90973214- | 90973199- | | |
| | | | | 90982309 | 90973218 | | |
| 696 | 84146 | ZNF644 | exon_06_nc.1 | chr1: | chr1: | AAUUACCAAUGGGGAUGAGG | + |
| | | | | 90973153- | 90973147- | | |
| | | | | 90973213 | 90973166 | | |
| 697 | 84146 | ZNF644 | intron_06 | chr1: | chr1: | GCCACUACAGAAAACUAUGG | + |
| | | | | 90941310- | 90972536- | | |
| | | | | 90972743 | 90972555 | | |
| 698 | 84146 | ZNF644 | intron_06 | chr1: | chr1: | GCAGCCACUACAGAAAACUA | + |
| | | | | 90941310- | 90972533- | | |
| | | | | 90972743 | 90972552 | | |
| 699 | 84146 | ZNF644 | intron_06 | chr1: | chr1: | CUGGUUUCUAUCUGAUUGAA | − |
| | | | | 90941310- | 90971842- | | |
| | | | | 90972743 | 90971861 | | |
| 700 | 84146 | ZNF644 | intron_06 | chr1: | chr1: | GUUUCUAUCUGAUUGAAAGG | − |
| | | | | 90941310- | 90971839- | | |
| | | | | 90972743 | 90971858 | | |
| 701 | 84146 | ZNF644 | intron_06 | chr1: | chr1: | UUCUCAAUCUCUUUUCUACC | − |
| | | | | 90941310- | 90971106- | | |
| | | | | 90972743 | 90971125 | | |
| 702 | 84146 | ZNF644 | intron_06 | chr1: | chr1: | CUGUAUGCCUUCAUUUAUCC | + |
| | | | | 90941310- | 90971085- | | |
| | | | | 90972743 | 90971104 | | |
| 703 | 84146 | ZNF644 | intron_06 | chr1: | chr1: | AGAUAAUCACUUAUAUACCC | − |
| | | | | 90941310- | 90970729- | | |
| | | | | 90972743 | 90970748 | | |
| 704 | 84146 | ZNF644 | intron_06 | chr1: | chr1: | GCAUAUACAACCAAAUACCA | + |
| | | | | 90941310- | 90970709- | | |
| | | | | 90972743 | 90970728 | | |
| 705 | 84146 | ZNF644 | intron_06 | chr1: | chr1: | CACUUAUAUACCCUGGUAUU | − |
| | | | | 90941310- | 90970722- | | |
| | | | | 90972743 | 90970741 | | |
| 706 | 84146 | ZNF644 | intron_06 | chr1: | chr1: | AGAUUGUGCAACUCCUCCCC | + |
| | | | | 90941310- | 90970402- | | |
| | | | | 90972743 | 90970421 | | |
| 707 | 84146 | ZNF644 | intron_06 | chr1: | chr1: | GCUGUGCUUCAGAUAAACUU | + |
| | | | | 90941310- | 90970362- | | |
| | | | | 90972743 | 90970381 | | |
| 708 | 84146 | ZNF644 | intron_06 | chr1: | chr1: | GCAGACUAAUAUGCUCAACU | + |
| | | | | 90941310- | 90969905- | | |
| | | | | 90972743 | 90969924 | | |
| 709 | 84146 | ZNF644 | intron_06 | chr1: | chr1: | AGGUCUACUGUAGAUUCUUA | + |
| | | | | 90941310- | 90969874- | | |
| | | | | 90972743 | 90969893 | | |
| 710 | 84146 | ZNF644 | intron_06 | chr1: | chr1: | UUCGCAUCCACUGGAGGUAC | + |
| | | | | 90941310- | 90969828- | | |
| | | | | 90972743 | 90969847 | | |
| 711 | 84146 | ZNF644 | intron_06 | chr1: | chr1: | ACUGAGGUUUUCGCAUCCAC | + |
| | | | | 90941310- | 90969819- | | |
| | | | | 90972743 | 90969838 | | |
| 712 | 84146 | ZNF644 | intron_06 | chr1: | chr1: | UUCAAUUACCUAGUCAACUA | + |
| | | | | 90941310- | 90969445- | | |
| | | | | 90972743 | 90969464 | | |
| 713 | 84146 | ZNF644 | intron_06 | chr1: | chr1: | GUUAUUACAUUCUAAAAGUA | + |
| | | | | 90941310- | 90968930- | | |
| | | | | 90972743 | 90968949 | | |
| 714 | 84146 | ZNF644 | intron_06 | chr1: | chr1: | GUGUGACUUUUGGUUUGCUG | − |
| | | | | 90941310- | 90968886- | | |
| | | | | 90972743 | 90968905 | | |
| 715 | 84146 | ZNF644 | intron_06 | chr1: | chr1: | UUGUUGUAUCAUUAGCCUUC | + |
| | | | | 90941310- | 90968553- | | |
| | | | | 90972743 | 90968572 | | |
| 716 | 84146 | ZNF644 | intron_06 | chr1: | chr1: | AUAUAUUUCGGUUUUGGGGG | + |
| | | | | 90941310- | 90968416- | | |
| | | | | 90972743 | 90968435 | | |
| 717 | 84146 | ZNF644 | intron_06 | chr1: | chr1: | AAUAUAUUUCGGUUUUGGGG | + |
| | | | | 90941310- | 90968415- | | |
| | | | | 90972743 | 90968434 | | |
| 718 | 84146 | ZNF644 | intron_06 | chr1: | chr1: | UAAUAUAUUUCGGUUUUGGG | + |
| | | | | 90941310- | 90968414- | | |
| | | | | 90972743 | 90968433 | | |
| 719 | 84146 | ZNF644 | intron_06 | chr1: | chr1: | CUCAUGAUCAGUUAUUUUGU | + |
| | | | | 90941310- | 90968196- | | |
| | | | | 90972743 | 90968215 | | |
| 720 | 84146 | ZNF644 | intron_06 | chr1: | chr1: | GAUCAUGAGGCAUGUGAAUU | − |
| | | | | 90941310- | 90968185- | | |
| | | | | 90972743 | 90968204 | | |
| 721 | 84146 | ZNF644 | intron_06 | chr1: | chr1: | GGAUCUUUUUGUGCACUCAC | − |
| | | | | 90941310- | 90967738- | | |
| | | | | 90972743 | 90967757 | | |
| 722 | 84146 | ZNF644 | intron_06 | chr1: | chr1: | CAGACACAUUGCAUAUUUCU | + |
| | | | | 90941310- | 90967695- | | |
| | | | | 90972743 | 90967714 | | |
| 723 | 84146 | ZNF644 | intron_06 | chr1: | chr1: | UCCUAUAAUUGACUGAUAUC | − |
| | | | | 90941310- | 90967629- | | |
| | | | | 90972743 | 90967648 | | |
| 724 | 84146 | ZNF644 | intron_06 | chr1: | chr1: | GGUAGUAGUCAACCUAAUGA | + |
| | | | | 90941310- | 90967565- | | |
| | | | | 90972743 | 90967584 | | |
| 725 | 84146 | ZNF644 | intron_06 | chr1: | chr1: | UCUGUAAGAGCAUUUGUCAA | − |
| | | | | 90941310- | 90967363- | | |
| | | | | 90972743 | 90967382 | | |
| 726 | 84146 | ZNF644 | intron_06 | chr1: | chr1: | UACAUGUAGUUCCCUUGGUC | + |
| | | | | 90941310- | 90967220- | | |
| | | | | 90972743 | 90967239 | | |
| 727 | 84146 | ZNF644 | intron_06 | chr1: | chr1: | CUCUGUACAUGUAGUUCCCU | + |
| | | | | 90941310- | 90967215- | | |
| | | | | 90972743 | 90967234 | | |
| 728 | 84146 | ZNF644 | intron_06 | chr1: | chr1: | AGCUGAGCAGCUCACGUGAU | + |
| | | | | 90941310- | 90966842- | | |
| | | | | 90972743 | 90966861 | | |
| 729 | 84146 | ZNF644 | intron_06 | chr1: | chr1: | UGGGUAACGGGCCAACUUCA | + |
| | | | | 90941310- | 90966773- | | |
| | | | | 90972743 | 90966792 | | |
| 730 | 84146 | ZNF644 | intron_06 | chr1: | chr1: | AGUGUUCCAUUUUGAGCCAU | + |
| | | | | 90941310- | 90966414- | | |
| | | | | 90972743 | 90966433 | | |
| 731 | 84146 | ZNF644 | intron_06 | chr1: | chr1: | CUUUCUCAAUGUACUCAUAU | + |
| | | | | 90941310- | 90966059- | | |
| | | | | 90972743 | 90966078 | | |
| 732 | 84146 | ZNF644 | intron_06 | chr1: | chr1: | GUUAUAGUCUGAAGUAGAGG | + |
| | | | | 90941310- | 90965476- | | |
| | | | | 90972743 | 90965495 | | |
| 733 | 84146 | ZNF644 | intron_06 | chr1: | chr1: | GAGGUUAUAGUCUGAAGUAG | + |
| | | | | 90941310- | 90965473- | | |
| | | | | 90972743 | 90965492 | | |
| 734 | 84146 | ZNF644 | intron_06 | chr1: | chr1: | AGACCCUAUAUUUCCAAGUG | + |
| | | | | 90941310- | 90965454- | | |
| | | | | 90972743 | 90965473 | | |
| 735 | 84146 | ZNF644 | intron_06 | chr1: | chr1: | UCCAAGACAAAAGUACAGUA | + |
| | | | | 90941310- | 90965228- | | |
| | | | | 90972743 | 90965247 | | |
| 736 | 84146 | ZNF644 | intron_06 | chr1: | chr1: | GAUAACAGAAAAAGCUAUAC | + |
| | | | | 90941310- | 90965112- | | |
| | | | | 90972743 | 90965131 | | |
| 737 | 84146 | ZNF644 | intron_06 | chr1: | chr1: | ACCCUUGUUAAGUAUGGGGA | + |
| | | | | 90941310- | 90965037- | | |
| | | | | 90972743 | 90965056 | | |
| 738 | 84146 | ZNF644 | intron_06 | chr1: | chr1: | UGUUACCCUUGUUAAGUAUG | + |
| | | | | 90941310- | 90965033- | | |
| | | | | 90972743 | 90965052 | | |
| 739 | 84146 | ZNF644 | intron_06 | chr1: | chr1: | CUGUUACCCUUGUUAAGUAU | + |
| | | | | 90941310- | 90965032- | | |
| | | | | 90972743 | 90965051 | | |
| 740 | 84146 | ZNF644 | intron_06 | chr1: | chr1: | UCUGUUACCCUUGUUAAGUA | + |
| | | | | 90941310- | 90965031- | | |
| | | | | 90972743 | 90965050 | | |
| 741 | 84146 | ZNF644 | intron_06 | chr1: | chr1: | CUCCUUCCCCAUACUUAACA | − |
| | | | | 90941310- | 90965042- | | |
| | | | | 90972743 | 90965061 | | |
| 742 | 84146 | ZNF644 | intron_06 | chr1: | chr1: | UCCUUCCCCAUACUUAACAA | − |
| | | | | 90941310- | 90965041- | | |
| | | | | 90972743 | 90965060 | | |
| 743 | 84146 | ZNF644 | intron_06 | chr1: | chr1: | AGAUUGUUAUGAAACGUUCC | − |
| | | | | 90941310- | 90965014- | | |
| | | | | 90972743 | 90965033 | | |
| 744 | 84146 | ZNF644 | intron_06 | chr1: | chr1: | UUAUGUCUUCUAAGGAAAUG | − |
| | | | | 90941310- | 90964937- | | |
| | | | | 90972743 | 90964956 | | |
| 745 | 84146 | ZNF644 | intron_06 | chr1: | chr1: | AUGUCUUCUAAGGAAAUGUG | − |
| | | | | 90941310- | 90964935- | | |
| | | | | 90972743 | 90964954 | | |
| 746 | 84146 | ZNF644 | intron_06 | chr1: | chr1: | AGCAACAGAAAAAGCUAUAC | + |
| | | | | 90941310- | 90964681- | | |
| | | | | 90972743 | 90964700 | | |
| 747 | 84146 | ZNF644 | intron_06 | chr1: | chr1: | GUUGCUUCAGUGUGGUAUUU | − |
| | | | | 90941310- | 90964667- | | |
| | | | | 90972743 | 90964686 | | |
| 748 | 84146 | ZNF644 | intron_06 | chr1: | chr1: | AAGAAUUUGUUACCACUUGG | − |
| | | | | 90941310- | 90964641- | | |
| | | | | 90972743 | 90964660 | | |
| 749 | 84146 | ZNF644 | intron_06 | chr1: | chr1: | AGCCACAUAGUGCCACCAAG | + |
| | | | | 90941310- | 90964626- | | |
| | | | | 90972743 | 90964645 | | |
| 750 | 84146 | ZNF644 | intron_06 | chr1: | chr1: | GCAAUUAACUAUACUGUUCU | − |
| | | | | 90941310- | 90964585- | | |
| | | | | 90972743 | 90964604 | | |
| 751 | 84146 | ZNF644 | intron_06 | chr1: | chr1: | CUACCCAACAAACUAAGCCU | + |
| | | | | 90941310- | 90964428- | | |
| | | | | 90972743 | 90964447 | | |
| 752 | 84146 | ZNF644 | intron_06 | chr1: | chr1: | UAAACCAAGGCUUAGUUUGU | − |
| | | | | 90941310- | 90964435- | | |
| | | | | 90972743 | 90964454 | | |
| 753 | 84146 | ZNF644 | intron_06 | chr1: | chr1: | CAAGGCUUAGUUUGUUGGGU | − |
| | | | | 90941310- | 90964430- | | |
| | | | | 90972743 | 90964449 | | |
| 754 | 84146 | ZNF644 | intron_06 | chr1: | chr1: | AAGGCUUAGUUUGUUGGGUA | − |
| | | | | 90941310- | 90964429- | | |
| | | | | 90972743 | 90964448 | | |
| 755 | 84146 | ZNF644 | intron_06 | chr1: | chr1: | AUAAAUGACACCUGACCUAA | + |
| | | | | 90941310- | 90964376- | | |
| | | | | 90972743 | 90964395 | | |
| 756 | 84146 | ZNF644 | intron_06 | chr1: | chr1: | AUAGAAACCUCCUUUAGGUC | − |
| | | | | 90941310- | 90964389- | | |
| | | | | 90972743 | 90964408 | | |
| 757 | 84146 | ZNF644 | intron_06 | chr1: | chr1: | CUCACAUUUAGACAAAGAAG | + |
| | | | | 90941310- | 90964339- | | |
| | | | | 90972743 | 90964358 | | |
| 758 | 84146 | ZNF644 | intron_06 | chr1: | chr1: | UCUCACAUUUAGACAAAGAA | + |
| | | | | 90941310- | 90964338- | | |
| | | | | 90972743 | 90964357 | | |
| 759 | 84146 | ZNF644 | intron_06 | chr1: | chr1: | GGUAUUAGAUGUCAAGUGCU | + |
| | | | | 90941310- | 90964016- | | |
| | | | | 90972743 | 90964035 | | |
| 760 | 84146 | ZNF644 | intron_06 | chr1: | chr1: | CACAAAUACCUUAACAAUUA | + |
| | | | | 90941310- | 90963995- | | |
| | | | | 90972743 | 90964014 | | |
| 761 | 84146 | ZNF644 | intron_06 | chr1: | chr1: | UUCUCGAUCUGUGGUGGUCA | + |
| | | | | 90941310- | 90963832- | | |
| | | | | 90972743 | 90963851 | | |
| 762 | 84146 | ZNF644 | intron_06 | chr1: | chr1: | ACAAUUUGUCAUGUUACUGU | − |
| | | | | 90941310- | 90963569- | | |
| | | | | 90972743 | 90963588 | | |
| 763 | 84146 | ZNF644 | intron_06 | chr1: | chr1: | UAUUCAAUUCCAUUUGGAUC | + |
| | | | | 90941310- | 90963269- | | |
| | | | | 90972743 | 90963288 | | |
| 764 | 84146 | ZNF644 | intron_06 | chr1: | chr1: | CAACCAGUACAUUUAUGAAA | + |
| | | | | 90941310- | 90963246- | | |
| | | | | 90972743 | 90963265 | | |
| 765 | 84146 | ZNF644 | intron_06 | chr1: | chr1: | ACAACCAGUACAUUUAUGAA | + |
| | | | | 90941310- | 90963245- | | |
| | | | | 90972743 | 90963264 | | |
| 766 | 84146 | ZNF644 | intron_06 | chr1: | chr1: | GAGAAGAGACGUUGCAAAAA | + |
| | | | | 90941310- | 90963214- | | |
| | | | | 90972743 | 90963233 | | |
| 767 | 84146 | ZNF644 | intron_06 | chr1: | chr1: | UUUGUUAAGGUGGUGAGGAU | + |
| | | | | 90941310- | 90963192- | | |
| | | | | 90972743 | 90963211 | | |
| 768 | 84146 | ZNF644 | intron_06 | chr1: | chr1: | AAAAGUACAGAAACUCCAAU | + |
| | | | | 90941310- | 90963100- | | |
| | | | | 90972743 | 90963119 | | |
| 769 | 84146 | ZNF644 | intron_06 | chr1: | chr1: | AAAUGUCCCACCCAGAUGGU | + |
| | | | | 90941310- | 90962780- | | |
| | | | | 90972743 | 90962799 | | |
| 770 | 84146 | ZNF644 | intron_06 | chr1: | chr1: | UGCCAAAUGUCCCACCCAGA | + |
| | | | | 90941310- | 90962776- | | |
| | | | | 90972743 | 90962795 | | |
| 771 | 84146 | ZNF644 | intron_06 | chr1: | chr1: | CUCACCCACUAGUGACACCC | + |
| | | | | 90941310- | 90962614- | | |
| | | | | 90972743 | 90962633 | | |
| 772 | 84146 | ZNF644 | intron_06 | chr1: | chr1: | AAGAACAAGGUAGGCCUUAG | + |
| | | | | 90941310- | 90962209- | | |
| | | | | 90972743 | 90962228 | | |
| 773 | 84146 | ZNF644 | intron_06 | chr1: | chr1: | GACUGAAAUUCUUUCUUCUC | − |
| | | | | 90941310- | 90962050- | | |
| | | | | 90972743 | 90962069 | | |
| 774 | 84146 | ZNF644 | intron_06 | chr1: | chr1: | UCAACUGGAAUCAAUACACU | + |
| | | | | 90941310- | 90961786- | | |
| | | | | 90972743 | 90961805 | | |
| 775 | 84146 | ZNF644 | intron_06 | chr1: | chr1: | CAUCCCUAAGACCUUAUUAG | + |
| | | | | 90941310- | 90961417- | | |
| | | | | 90972743 | 90961436 | | |
| 776 | 84146 | ZNF644 | intron_06 | chr1: | chr1: | GUGUGAGUUGUGGGGCCCAG | + |
| | | | | 90941310- | 90961393- | | |
| | | | | 90972743 | 90961412 | | |
| 777 | 84146 | ZNF644 | intron_06 | chr1: | chr1: | GUUUGAUUAAAUUAGGUCAA | + |
| | | | | 90941310- | 90961334- | | |
| | | | | 90972743 | 90961353 | | |
| 778 | 84146 | ZNF644 | intron_06 | chr1: | chr1: | AGUUUGAUUAAAUUAGGUCA | + |
| | | | | 90941310- | 90961333- | | |
| | | | | 90972743 | 90961352 | | |
| 779 | 84146 | ZNF644 | intron_06 | chr1: | chr1: | AUUAACAGUUUGAUUAAAUU | + |
| | | | | 90941310- | 90961327- | | |
| | | | | 90972743 | 90961346 | | |
| 780 | 84146 | ZNF644 | intron_06 | chr1: | chr1: | AGUAAGGGGUACAGUAUGGA | + |
| | | | | 90941310- | 90961205- | | |
| | | | | 90972743 | 90961224 | | |
| 781 | 84146 | ZNF644 | intron_06 | chr1: | chr1: | GUGGCAAAUAAGUUGAGUAA | + |
| | | | | 90941310- | 90961190- | | |
| | | | | 90972743 | 90961209 | | |
| 782 | 84146 | ZNF644 | intron_06 | chr1: | chr1: | AGUGGCAAAUAAGUUGAGUA | + |
| | | | | 90941310- | 90961189- | | |
| | | | | 90972743 | 90961208 | | |
| 783 | 84146 | ZNF644 | intron_06 | chr1: | chr1: | CAUGGUGGCUAAAAUAUAUA | + |
| | | | | 90941310- | 90961145- | | |
| | | | | 90972743 | 90961164 | | |
| 784 | 84146 | ZNF644 | intron_06 | chr1: | chr1: | ACGAUGAUGACGUUUCAUGG | + |
| | | | | 90941310- | 90961130- | | |
| | | | | 90972743 | 90961149 | | |
| 785 | 84146 | ZNF644 | intron_06 | chr1: | chr1: | AAAACGAUGAUGACGUUUCA | + |
| | | | | 90941310- | 90961127- | | |
| | | | | 90972743 | 90961146 | | |
| 786 | 84146 | ZNF644 | intron_06 | chr1: | chr1: | CACUACAGACCUACACUAAA | + |
| | | | | 90941310- | 90960991- | | |
| | | | | 90972743 | 90961010 | | |
| 787 | 84146 | ZNF644 | intron_06 | chr1: | chr1: | CAAGAAUCAUCCAAAAGUGA | + |
| | | | | 90941310- | 90960923- | | |
| | | | | 90972743 | 90960942 | | |
| 788 | 84146 | ZNF644 | intron_06 | chr1: | chr1: | ACAAGAAUCAUCCAAAAGUG | + |
| | | | | 90941310- | 90960922- | | |
| | | | | 90972743 | 90960941 | | |
| 789 | 84146 | ZNF644 | intron_06 | chr1: | chr1: | AAUUUCUCAACAGAAACAGA | + |
| | | | | 90941310- | 90960837- | | |
| | | | | 90972743 | 90960856 | | |
| 790 | 84146 | ZNF644 | intron_06 | chr1: | chr1: | GUCCUGACGACACCCAAUCU | + |
| | | | | 90941310- | 90960610- | | |
| | | | | 90972743 | 90960629 | | |
| 791 | 84146 | ZNF644 | intron_06 | chr1: | chr1: | GUACACUCUAUGGAUAAAGA | + |
| | | | | 90941310- | 90960482- | | |
| | | | | 90972743 | 90960501 | | |
| 792 | 84146 | ZNF644 | intron_06 | chr1: | chr1: | GUCUUCAAAUGUACACUCUA | + |
| | | | | 90941310- | 90960472- | | |
| | | | | 90972743 | 90960491 | | |
| 793 | 84146 | ZNF644 | intron_06 | chr1: | chr1: | GUGUCUGACUGACAGCAAUA | + |
| | | | | 90941310- | 90960413- | | |
| | | | | 90972743 | 90960432 | | |
| 794 | 84146 | ZNF644 | intron_06 | chr1: | chr1: | AGCUGCAAAAGUUAUGAACG | + |
| | | | | 90941310- | 90960322- | | |
| | | | | 90972743 | 90960341 | | |
| 795 | 84146 | ZNF644 | intron_06 | chr1: | chr1: | UACUCGCUGAAUUGGUAAGA | + |
| | | | | 90941310- | 90960262- | | |
| | | | | 90972743 | 90960281 | | |
| 796 | 84146 | ZNF644 | intron_06 | chr1: | chr1: | GCCGUUUCCUUUACAUAAAA | + |
| | | | | 90941310- | 90960171- | | |
| | | | | 90972743 | 90960190 | | |
| 797 | 84146 | ZNF644 | intron_06 | chr1: | chr1: | CUUGCACCAUCCCACUCAGA | + |
| | | | | 90941310- | 90959957- | | |
| | | | | 90972743 | 90959976 | | |
| 798 | 84146 | ZNF644 | intron_06 | chr1: | chr1: | UCUUGCACCAUCCCACUCAG | + |
| | | | | 90941310- | 90959956- | | |
| | | | | 90972743 | 90959975 | | |
| 799 | 84146 | ZNF644 | intron_06 | chr1: | chr1: | GGUUUUAAAUUGCUAUUCUA | + |
| | | | | 90941310- | 90959921- | | |
| | | | | 90972743 | 90959940 | | |
| 800 | 84146 | ZNF644 | intron_06 | chr1: | chr1: | GAAUACGCAGUGUUUUUAUG | + |
| | | | | 90941310- | 90959741- | | |
| | | | | 90972743 | 90959760 | | |
| 801 | 84146 | ZNF644 | intron_06 | chr1: | chr1: | UGAAUACGCAGUGUUUUUAU | + |
| | | | | 90941310- | 90959740- | | |
| | | | | 90972743 | 90959759 | | |
| 802 | 84146 | ZNF644 | intron_06 | chr1: | chr1: | AUGAAUACGCAGUGUUUUUA | + |
| | | | | 90941310- | 90959739- | | |
| | | | | 90972743 | 90959758 | | |
| 803 | 84146 | ZNF644 | intron_06 | chr1: | chr1: | CUUUACCAGGUGAUGGAAGG | + |
| | | | | 90941310- | 90959695- | | |
| | | | | 90972743 | 90959714 | | |
| 804 | 84146 | ZNF644 | intron_06 | chr1: | chr1: | ACUUUACCAGGUGAUGGAAG | + |
| | | | | 90941310- | 90959694- | | |
| | | | | 90972743 | 90959713 | | |
| 805 | 84146 | ZNF644 | intron_06 | chr1: | chr1: | UACUUUACCAGGUGAUGGAA | + |
| | | | | 90941310- | 90959693- | | |
| | | | | 90972743 | 90959712 | | |
| 806 | 84146 | ZNF644 | intron_06 | chr1: | chr1: | CAAUGCCUAGAUAUACAUCC | + |
| | | | | 90941310- | 90959387- | | |
| | | | | 90972743 | 90959406 | | |
| 807 | 84146 | ZNF644 | intron_06 | chr1: | chr1: | AUAAGUAUGGAUAAUAAGUU | + |
| | | | | 90941310- | 90959318- | | |
| | | | | 90972743 | 90959337 | | |
| 808 | 84146 | ZNF644 | intron_06 | chr1: | chr1: | GAUCCUCAUGACUCUUGAUU | + |
| | | | | 90941310- | 90958883- | | |
| | | | | 90972743 | 90958902 | | |
| 809 | 84146 | ZNF644 | intron_06 | chr1: | chr1: | UUGGAAGCCUGUCUCACACC | + |
| | | | | 90941310- | 90958782- | | |
| | | | | 90972743 | 90958801 | | |
| 810 | 84146 | ZNF644 | intron_06 | chr1: | chr1: | UCAAUGUGGCAGUAGAAGCA | + |
| | | | | 90941310- | 90958593- | | |
| | | | | 90972743 | 90958612 | | |
| 811 | 84146 | ZNF644 | intron_06 | chr1: | chr1: | AUCAAUGUGGCAGUAGAAGC | + |
| | | | | 90941310- | 90958592- | | |
| | | | | 90972743 | 90958611 | | |
| 812 | 84146 | ZNF644 | intron_06 | chr1: | chr1: | ACAUUGCUAGAAAAACUUAA | + |
| | | | | 90941310- | 90958308- | | |
| | | | | 90972743 | 90958327 | | |
| 813 | 84146 | ZNF644 | intron_06 | chr1: | chr1: | GUGUAACCUCAUGACCAGCC | + |
| | | | | 90941310- | 90958058- | | |
| | | | | 90972743 | 90958077 | | |
| 814 | 84146 | ZNF644 | intron_06 | chr1: | chr1: | UGGAAAAAUCCAGGACUAUG | + |
| | | | | 90941310- | 90957173- | | |
| | | | | 90972743 | 90957192 | | |
| 815 | 84146 | ZNF644 | intron_06 | chr1: | chr1: | AACAGAAUACUAUGAAUAAC | + |
| | | | | 90941310- | 90957004- | | |
| | | | | 90972743 | 90957023 | | |
| 816 | 84146 | ZNF644 | intron_06 | chr1: | chr1: | AAGCUGAGAUAAACUUACAC | − |
| | | | | 90941310- | 90956421- | | |
| | | | | 90972743 | 90956440 | | |
| 817 | 84146 | ZNF644 | intron_06 | chr1: | chr1: | UCAGUUCACUAUCUUGGAUU | + |
| | | | | 90941310- | 90956139- | | |
| | | | | 90972743 | 90956158 | | |
| 818 | 84146 | ZNF644 | intron_06 | chr1: | chr1: | UCCUCACUAAGCUUAAUUCU | + |
| | | | | 90941310- | 90955922- | | |
| | | | | 90972743 | 90955941 | | |
| 819 | 84146 | ZNF644 | intron_06 | chr1: | chr1: | UGAGCUUUUAUGUUAUAACA | + |
| | | | | 90941310- | 90955556- | | |
| | | | | 90972743 | 90955575 | | |
| 820 | 84146 | ZNF644 | intron_06 | chr1: | chr1: | UUGUCAGAGCUAGAUCUCCU | + |
| | | | | 90941310- | 90955491- | | |
| | | | | 90972743 | 90955510 | | |
| 821 | 84146 | ZNF644 | intron_06 | chr1: | chr1: | CAGGCUUCAGUUUUGAAGCC | + |
| | | | | 90941310- | 90955360- | | |
| | | | | 90972743 | 90955379 | | |
| 822 | 84146 | ZNF644 | intron_06 | chr1: | chr1: | UUCAUAAACCAUGCUGUAAC | + |
| | | | | 90941310- | 90955179- | | |
| | | | | 90972743 | 90955198 | | |
| 823 | 84146 | ZNF644 | intron_06 | chr1: | chr1: | UCAAUGAGCAUAUUUCGAAA | + |
| | | | | 90941310- | 90955117- | | |
| | | | | 90972743 | 90955136 | | |
| 824 | 84146 | ZNF644 | intron_06 | chr1: | chr1: | CUUAAUAUUGACAAUCAGAG | + |
| | | | | 90941310- | 90954029- | | |
| | | | | 90972743 | 90954048 | | |
| 825 | 84146 | ZNF644 | intron_06 | chr1: | chr1: | UAAAGUAUUCUAUUGUAGCU | + |
| | | | | 90941310- | 90953618- | | |
| | | | | 90972743 | 90953637 | | |
| 826 | 84146 | ZNF644 | intron_06 | chr1: | chr1: | UUAAAGUAUUCUAUUGUAGC | + |
| | | | | 90941310- | 90953617- | | |
| | | | | 90972743 | 90953636 | | |
| 827 | 84146 | ZNF644 | intron_06 | chr1: | chr1: | GCUAUUAUGUCUAAAAAGCA | + |
| | | | | 90941310- | 90953586- | | |
| | | | | 90972743 | 90953605 | | |
| 828 | 84146 | ZNF644 | intron_06 | chr1: | chr1: | CAUGGAGAGGGGAACACACU | + |
| | | | | 90941310- | 90953351- | | |
| | | | | 90972743 | 90953370 | | |
| 829 | 84146 | ZNF644 | intron_06 | chr1: | chr1: | UACAUUUAACAGACAUGGAG | + |
| | | | | 90941310- | 90953338- | | |
| | | | | 90972743 | 90953357 | | |
| 830 | 84146 | ZNF644 | intron_06 | chr1: | chr1: | GAUAGCAUGAGAUUAGCUCU | − |
| | | | | 90941310- | 90953048- | | |
| | | | | 90972743 | 90953067 | | |
| 831 | 84146 | ZNF644 | intron_06 | chr1: | chr1: | GAGAUUAGCUCUUGGACAUC | − |
| | | | | 90941310- | 90953040- | | |
| | | | | 90972743 | 90953059 | | |
| 832 | 84146 | ZNF644 | intron_06 | chr1: | chr1: | AAGAGACAACAAUUAGACUG | + |
| | | | | 90941310- | 90952991- | | |
| | | | | 90972743 | 90953010 | | |
| 833 | 84146 | ZNF644 | intron_06 | chr1: | chr1: | CACAUCAAGCCAAAGCUUCU | + |
| | | | | 90941310- | 90952841- | | |
| | | | | 90972743 | 90952860 | | |
| 834 | 84146 | ZNF644 | intron_06 | chr1: | chr1: | ACACAUCAAGCCAAAGCUUC | + |
| | | | | 90941310- | 90952840- | | |
| | | | | 90972743 | 90952859 | | |
| 835 | 84146 | ZNF644 | intron_06 | chr1: | chr1: | CAUCUAGUUGGAAUCCCGGA | + |
| | | | | 90941310- | 90952746- | | |
| | | | | 90972743 | 90952765 | | |
| 836 | 84146 | ZNF644 | intron_06 | chr1: | chr1: | CAUACAUUCCAACAUCUAGU | + |
| | | | | 90941310- | 90952734- | | |
| | | | | 90972743 | 90952753 | | |
| 837 | 84146 | ZNF644 | intron_06 | chr1: | chr1: | AAAGUAUAUUGAAUGAAGCA | + |
| | | | | 90941310- | 90952646- | | |
| | | | | 90972743 | 90952665 | | |
| 838 | 84146 | ZNF644 | intron_06 | chr1: | chr1: | UACAUAGUGGCCAGAGCAUA | − |
| | | | | 90941310- | 90952566- | | |
| | | | | 90972743 | 90952585 | | |
| 839 | 84146 | ZNF644 | intron_06 | chr1: | chr1: | UUCUGUUGGAAGCUUCUCAG | − |
| | | | | 90941310- | 90952526- | | |
| | | | | 90972743 | 90952545 | | |
| 840 | 84146 | ZNF644 | intron_06 | chr1: | chr1: | GAGAGGUCUAUACAACAACU | + |
| | | | | 90941310- | 90952447- | | |
| | | | | 90972743 | 90952466 | | |
| 841 | 84146 | ZNF644 | intron_06 | chr1: | chr1: | CUGAUCAAACAUAUAGGGAG | + |
| | | | | 90941310- | 90952430- | | |
| | | | | 90972743 | 90952449 | | |
| 842 | 84146 | ZNF644 | intron_06 | chr1: | chr1: | CUUGGUUUAGCUGUAAAGAC | + |
| | | | | 90941310- | 90952186- | | |
| | | | | 90972743 | 90952205 | | |
| 843 | 84146 | ZNF644 | intron_06 | chr1: | chr1: | GAAGCCAUUUUUACUGACAA | + |
| | | | | 90941310- | 90952158- | | |
| | | | | 90972743 | 90952177 | | |
| 844 | 84146 | ZNF644 | intron_06 | chr1: | chr1: | UAAUUCUAAGACUGGGAGUC | − |
| | | | | 90941310- | 90952076- | | |
| | | | | 90972743 | 90952095 | | |
| 845 | 84146 | ZNF644 | intron_06 | chr1: | chr1: | UUAAUCAAAAAACAGCUCAA | + |
| | | | | 90941310- | 90951995- | | |
| | | | | 90972743 | 90952014 | | |
| 846 | 84146 | ZNF644 | intron_06 | chr1: | chr1: | GUUAAUCAAAAAACAGCUCA | + |
| | | | | 90941310- | 90951994- | | |
| | | | | 90972743 | 90952013 | | |
| 847 | 84146 | ZNF644 | intron_06 | chr1: | chr1: | CACAUAAUAGGAAAAUUUGA | + |
| | | | | 90941310- | 90951958- | | |
| | | | | 90972743 | 90951977 | | |
| 848 | 84146 | ZNF644 | intron_06 | chr1: | chr1: | AAUGGCCUUCUGUCUGCCUA | − |
| | | | | 90941310- | 90951929- | | |
| | | | | 90972743 | 90951948 | | |
| 849 | 84146 | ZNF644 | intron_06 | chr1: | chr1: | CUUGCUAACUGAAUACCUCA | + |
| | | | | 90941310- | 90951805- | | |
| | | | | 90972743 | 90951824 | | |
| 850 | 84146 | ZNF644 | intron_06 | chr1: | chr1: | CCUCUAAUACUAAGUUUCAC | + |
| | | | | 90941310- | 90951765- | | |
| | | | | 90972743 | 90951784 | | |
| 851 | 84146 | ZNF644 | intron_06 | chr1: | chr1: | AUAUGUUCUCUACUAGCGUA | + |
| | | | | 90941310- | 90951453- | | |
| | | | | 90972743 | 90951472 | | |
| 852 | 84146 | ZNF644 | intron_06 | chr1: | chr1: | ACUAAUCUGGUAUUAGUACA | + |
| | | | | 90941310- | 90951166- | | |
| | | | | 90972743 | 90951185 | | |
| 853 | 84146 | ZNF644 | intron_06 | chr1: | chr1: | CUGCUAUACCCAGAUCCACU | + |
| | | | | 90941310- | 90951130- | | |
| | | | | 90972743 | 90951149 | | |
| 854 | 84146 | ZNF644 | intron_06 | chr1: | chr1: | CUCAUCUAUAUCAGUUGUUC | + |
| | | | | 90941310- | 90950988- | | |
| | | | | 90972743 | 90951007 | | |
| 855 | 84146 | ZNF644 | intron_06 | chr1: | chr1: | ACUAAGGUUGAUCUGAACAG | + |
| | | | | 90941310- | 90950734- | | |
| | | | | 90972743 | 90950753 | | |
| 856 | 84146 | ZNF644 | intron_06 | chr1: | chr1: | GCUUUAAACGAUUCUCCCAA | + |
| | | | | 90941310- | 90950650- | | |
| | | | | 90972743 | 90950669 | | |
| 857 | 84146 | ZNF644 | intron_06 | chr1: | chr1: | AGAGAUAUUCAUGAAUCUCU | + |
| | | | | 90941310- | 90950540- | | |
| | | | | 90972743 | 90950559 | | |
| 858 | 84146 | ZNF644 | intron_06 | chr1: | chr1: | AGGAUAUUAAGUACUAGGCC | + |
| | | | | 90941310- | 90950486- | | |
| | | | | 90972743 | 90950505 | | |
| 859 | 84146 | ZNF644 | intron_06 | chr1: | chr1: | ACCUGAGGAUAUUAAGUACU | + |
| | | | | 90941310- | 90950481- | | |
| | | | | 90972743 | 90950500 | | |
| 860 | 84146 | ZNF644 | intron_06 | chr1: | chr1: | GCCUAGUACUUAAUAUCCUC | − |
| | | | | 90941310- | 90950485- | | |
| | | | | 90972743 | 90950504 | | |
| 861 | 84146 | ZNF644 | intron_06 | chr1: | chr1: | AUACUAGUUUACAGAACCUG | + |
| | | | | 90941310- | 90950466- | | |
| | | | | 90972743 | 90950485 | | |
| 862 | 84146 | ZNF644 | intron_06 | chr1: | chr1: | CACAAUCUGCUUUUGGGAGG | − |
| | | | | 90941310- | 90950430- | | |
| | | | | 90972743 | 90950449 | | |
| 863 | 84146 | ZNF644 | intron_06 | chr1: | chr1: | AAAAAAUAGCGCUGGGGCCG | + |
| | | | | 90941310- | 90949981- | | |
| | | | | 90972743 | 90950000 | | |
| 864 | 84146 | ZNF644 | intron_06 | chr1: | chr1: | GAUUCAAAAAAAUAGCGCUG | + |
| | | | | 90941310- | 90949975- | | |
| | | | | 90972743 | 90949994 | | |
| 865 | 84146 | ZNF644 | intron_06 | chr1: | chr1: | UGAUUCAAAAAAAUAGCGCU | + |
| | | | | 90941310- | 90949974- | | |
| | | | | 90972743 | 90949993 | | |
| 866 | 84146 | ZNF644 | intron_06 | chr1: | chr1: | UUGAUUCAAAAAAAUAGCGC | + |
| | | | | 90941310- | 90949973- | | |
| | | | | 90972743 | 90949992 | | |
| 867 | 84146 | ZNF644 | intron_06 | chr1: | chr1: | GAUUCAGGAACGGUGUUUGG | − |
| | | | | 90941310- | 90949935- | | |
| | | | | 90972743 | 90949954 | | |
| 868 | 84146 | ZNF644 | intron_06 | chr1: | chr1: | CAUUCUCCUUAGCCACAUAC | − |
| | | | | 90941310- | 90949902- | | |
| | | | | 90972743 | 90949921 | | |
| 869 | 84146 | ZNF644 | intron_06 | chr1: | chr1: | AUUCUCCUUAGCCACAUACU | − |
| | | | | 90941310- | 90949901- | | |
| | | | | 90972743 | 90949920 | | |
| 870 | 84146 | ZNF644 | intron_06 | chr1: | chr1: | GCAAUAGGAUUCCCAGUAUG | + |
| | | | | 90941310- | 90949887- | | |
| | | | | 90972743 | 90949906 | | |
| 871 | 84146 | ZNF644 | intron_06 | chr1: | chr1: | AUUUGCGUGUUUUUAGCAAU | + |
| | | | | 90941310- | 90949872- | | |
| | | | | 90972743 | 90949891 | | |
| 872 | 84146 | ZNF644 | intron_06 | chr1: | chr1: | GUAAAGGUCUGGAGCAUCAU | + |
| | | | | 90941310- | 90949761- | | |
| | | | | 90972743 | 90949780 | | |
| 873 | 84146 | ZNF644 | intron_06 | chr1: | chr1: | UAUUACAAGAAAUAUAUUGA | + |
| | | | | 90941310- | 90949682- | | |
| | | | | 90972743 | 90949701 | | |
| 874 | 84146 | ZNF644 | intron_06 | chr1: | chr1: | UUCUACAAGUAUCUUAAUUU | + |
| | | | | 90941310- | 90949423- | | |
| | | | | 90972743 | 90949442 | | |
| 875 | 84146 | ZNF644 | intron_06 | chr1: | chr1: | AGUUUGAAUGUAUAUAAUAC | − |
| | | | | 90941310- | 90949150- | | |
| | | | | 90972743 | 90949169 | | |
| 876 | 84146 | ZNF644 | intron_06 | chr1: | chr1: | CAAUUUAAUCUAUUGAAGGA | + |
| | | | | 90941310- | 90949061- | | |
| | | | | 90972743 | 90949080 | | |
| 877 | 84146 | ZNF644 | intron_06 | chr1: | chr1: | UCAGUGAAAUAAUUCAAUCC | − |
| | | | | 90941310- | 90949037- | | |
| | | | | 90972743 | 90949056 | | |
| 878 | 84146 | ZNF644 | intron_06 | chr1: | chr1: | ACUUGAAUAAUAUGACAUCC | + |
| | | | | 90941310- | 90949016- | | |
| | | | | 90972743 | 90949035 | | |
| 879 | 84146 | ZNF644 | intron_06 | chr1: | chr1: | UUCGUUAUGUUCCAAGCAUC | − |
| | | | | 90941310- | 90948805- | | |
| | | | | 90972743 | 90948824 | | |
| 880 | 84146 | ZNF644 | intron_06 | chr1: | chr1: | AAGCAUCAGGCUAGACAUGC | − |
| | | | | 90941310- | 90948792- | | |
| | | | | 90972743 | 90948811 | | |
| 881 | 84146 | ZNF644 | intron_06 | chr1: | chr1: | CAUGCUGGUAAGGGUUCUGU | − |
| | | | | 90941310- | 90948777- | | |
| | | | | 90972743 | 90948796 | | |
| 882 | 84146 | ZNF644 | intron_06 | chr1: | chr1: | CCCGCACUUGACACCAUCUA | + |
| | | | | 90941310- | 90948541- | | |
| | | | | 90972743 | 90948560 | | |
| 883 | 84146 | ZNF644 | intron_06 | chr1: | chr1: | UCCGCAUUAACAAUCUGUUA | − |
| | | | | 90941310- | 90948438- | | |
| | | | | 90972743 | 90948457 | | |
| 884 | 84146 | ZNF644 | intron_06 | chr1: | chr1: | UGCAAACAUGGUGGCUUUAA | + |
| | | | | 90941310- | 90948379- | | |
| | | | | 90972743 | 90948398 | | |
| 885 | 84146 | ZNF644 | intron_06 | chr1: | chr1: | CCCAACCAUGUAUAUUCCAC | + |
| | | | | 90941310- | 90948306- | | |
| | | | | 90972743 | 90948325 | | |
| 886 | 84146 | ZNF644 | intron_06 | chr1: | chr1: | UCCUGUGGAAUAUACAUGGU | − |
| | | | | 90941310- | 90948310- | | |
| | | | | 90972743 | 90948329 | | |
| 887 | 84146 | ZNF644 | intron_06 | chr1: | chr1: | CUGUGGAAUAUACAUGGUUG | − |
| | | | | 90941310- | 90948308- | | |
| | | | | 90972743 | 90948327 | | |
| 888 | 84146 | ZNF644 | intron_06 | chr1: | chr1: | AUCAUUGUCAUUGUAAGUCA | + |
| | | | | 90941310- | 90947960- | | |
| | | | | 90972743 | 90947979 | | |
| 889 | 84146 | ZNF644 | intron_06 | chr1: | chr1: | AUAAACAGCAGACCCAUCAU | − |
| | | | | 90941310- | 90947588- | | |
| | | | | 90972743 | 90947607 | | |
| 890 | 84146 | ZNF644 | intron_06 | chr1: | chr1: | GGUAUUCUUUAACCUAUGAU | + |
| | | | | 90941310- | 90947573- | | |
| | | | | 90972743 | 90947592 | | |
| 891 | 84146 | ZNF644 | intron_06 | chr1: | chr1: | UGGUAUUCUUUAACCUAUGA | + |
| | | | | 90941310- | 90947572- | | |
| | | | | 90972743 | 90947591 | | |
| 892 | 84146 | ZNF644 | intron_06 | chr1: | chr1: | GGCAUAAUACUUUCUGUGGA | − |
| | | | | 90941310- | 90947524- | | |
| | | | | 90972743 | 90947543 | | |
| 893 | 84146 | ZNF644 | intron_06 | chr1: | chr1: | CAAAGUUGACACACUGCCCC | − |
| | | | | 90941310- | 90947501- | | |
| | | | | 90972743 | 90947520 | | |
| 894 | 84146 | ZNF644 | intron_06 | chr1: | chr1: | AUGUCAAUCCCAUACUCCAG | + |
| | | | | 90941310- | 90947482- | | |
| | | | | 90972743 | 90947501 | | |
| 895 | 84146 | ZNF644 | intron_06 | chr1: | chr1: | GACACACUGCCCCUGGAGUA | − |
| | | | | 90941310- | 90947494- | | |
| | | | | 90972743 | 90947513 | | |
| 896 | 84146 | ZNF644 | intron_06 | chr1: | chr1: | ACACACUGCCCCUGGAGUAU | − |
| | | | | 90941310- | 90947493- | | |
| | | | | 90972743 | 90947512 | | |
| 897 | 84146 | ZNF644 | intron_06 | chr1: | chr1: | AGUAUGGGAUUGACAUACUU | − |
| | | | | 90941310- | 90947478- | | |
| | | | | 90972743 | 90947497 | | |
| 898 | 84146 | ZNF644 | intron_06 | chr1: | chr1: | UGACCACAUUUGUGUAACAU | + |
| | | | | 90941310- | 90947387- | | |
| | | | | 90972743 | 90947406 | | |
| 899 | 84146 | ZNF644 | intron_06 | chr1: | chr1: | UGACCCAUGUUACACAAAUG | − |
| | | | | 90941310- | 90947393- | | |
| | | | | 90972743 | 90947412 | | |
| 900 | 84146 | ZNF644 | intron_06 | chr1: | chr1: | GUAAUAGACAUAUAUGCACG | + |
| | | | | 90941310- | 90947294- | | |
| | | | | 90972743 | 90947313 | | |
| 901 | 84146 | ZNF644 | intron_06 | chr1: | chr1: | CAGUUGCAGAAUUCUUCAUU | − |
| | | | | 90941310- | 90947151- | | |
| | | | | 90972743 | 90947170 | | |
| 902 | 84146 | ZNF644 | intron_06 | chr1: | chr1: | GCAGGUAACUGUGUGGAUGA | + |
| | | | | 90941310- | 90947009- | | |
| | | | | 90972743 | 90947028 | | |
| 903 | 84146 | ZNF644 | intron_06 | chr1: | chr1: | CCCAUGUGGUAAACGGAAGC | + |
| | | | | 90941310- | 90946991- | | |
| | | | | 90972743 | 90947010 | | |
| 904 | 84146 | ZNF644 | intron_06 | chr1: | chr1: | AGAAGCUCCCAUGUGGUAAA | + |
| | | | | 90941310- | 90946984- | | |
| | | | | 90972743 | 90947003 | | |
| 905 | 84146 | ZNF644 | intron_06 | chr1: | chr1: | ACCUGCUUCCGUUUACCACA | − |
| | | | | 90941310- | 90946995- | | |
| | | | | 90972743 | 90947014 | | |
| 906 | 84146 | ZNF644 | intron_06 | chr1: | chr1: | CCUGCUUCCGUUUACCACAU | − |
| | | | | 90941310- | 90946994- | | |
| | | | | 90972743 | 90947013 | | |
| 907 | 84146 | ZNF644 | intron_06 | chr1: | chr1: | CAUCUAAAGAAGCUCCCAUG | + |
| | | | | 90941310- | 90946977- | | |
| | | | | 90972743 | 90946996 | | |
| 908 | 84146 | ZNF644 | intron_06 | chr1: | chr1: | GUUAUUAGGUGUUUAUGGUC | − |
| | | | | 90941310- | 90946906- | | |
| | | | | 90972743 | 90946925 | | |
| 909 | 84146 | ZNF644 | intron_06 | chr1: | chr1: | AAGGCAGAUGUUACAAAGUC | + |
| | | | | 90941310- | 90946878- | | |
| | | | | 90972743 | 90946897 | | |
| 910 | 84146 | ZNF644 | intron_06 | chr1: | chr1: | AUCCUCACUUGUUAACAGGA | + |
| | | | | 90941310- | 90946859- | | |
| | | | | 90972743 | 90946878 | | |
| 911 | 84146 | ZNF644 | intron_06 | chr1: | chr1: | UACCAUCCUCACUUGUUAAC | + |
| | | | | 90941310- | 90946855- | | |
| | | | | 90972743 | 90946874 | | |
| 912 | 84146 | ZNF644 | intron_06 | chr1: | chr1: | UGCCUUCCUGUUAACAAGUG | − |
| | | | | 90941310- | 90946864- | | |
| | | | | 90972743 | 90946883 | | |
| 913 | 84146 | ZNF644 | intron_06 | chr1: | chr1: | UAACAAGUGAGGAUGGUACA | − |
| | | | | 90941310- | 90946853- | | |
| | | | | 90972743 | 90946872 | | |
| 914 | 84146 | ZNF644 | intron_06 | chr1: | chr1: | UCAGGAACAAGCUGUUCAAC | − |
| | | | | 90941310- | 90946828- | | |
| | | | | 90972743 | 90946847 | | |
| 915 | 84146 | ZNF644 | intron_06 | chr1: | chr1: | UCAUGAUACAGUUAGCUCUG | − |
| | | | | 90941310- | 90946680- | | |
| | | | | 90972743 | 90946699 | | |
| 916 | 84146 | ZNF644 | intron_06 | chr1: | chr1: | UUAGCUCUGUGGCAGUGUGU | − |
| | | | | 90941310- | 90946669- | | |
| | | | | 90972743 | 90946688 | | |
| 917 | 84146 | ZNF644 | intron_06 | chr1: | chr1: | ACAGAUCAUAAGUUAACUAA | − |
| | | | | 90941310- | 90946603- | | |
| | | | | 90972743 | 90946622 | | |
| 918 | 84146 | ZNF644 | intron_06 | chr1: | chr1: | UUUUAAAGUCAGUCUCAUGC | − |
| | | | | 90941310- | 90946329- | | |
| | | | | 90972743 | 90946348 | | |
| 919 | 84146 | ZNF644 | intron_06 | chr1: | chr1: | AUCCAGGUAAUAACUUAUUC | + |
| | | | | 90941310- | 90946251- | | |
| | | | | 90972743 | 90946270 | | |
| 920 | 84146 | ZNF644 | intron_06 | chr1: | chr1: | AUCCAGAAUAAGUUAUUACC | − |
| | | | | 90941310- | 90946256- | | |
| | | | | 90972743 | 90946275 | | |
| 921 | 84146 | ZNF644 | intron_06 | chr1: | chr1: | GAAGACAGAAUGUAUCAUCC | + |
| | | | | 90941310- | 90946235- | | |
| | | | | 90972743 | 90946254 | | |
| 922 | 84146 | ZNF644 | intron_06 | chr1: | chr1: | GUGUGCACUAGGUGAGAUUU | − |
| | | | | 90941310- | 90946208- | | |
| | | | | 90972743 | 90946227 | | |
| 923 | 84146 | ZNF644 | intron_06 | chr1: | chr1: | ACAUUGUGUAUGGUAUAUAU | − |
| | | | | 90941310- | 90946172- | | |
| | | | | 90972743 | 90946191 | | |
| 924 | 84146 | ZNF644 | intron_06 | chr1: | chr1: | AGUUAGUAAUGUAUGAGAUU | + |
| | | | | 90941310- | 90946051- | | |
| | | | | 90972743 | 90946070 | | |
| 925 | 84146 | ZNF644 | intron_06 | chr1: | chr1: | CAGUUAGUAAUGUAUGAGAU | + |
| | | | | 90941310- | 90946050- | | |
| | | | | 90972743 | 90946069 | | |
| 926 | 84146 | ZNF644 | intron_06 | chr1: | chr1: | GAAGUUGUCAUGUGUUCACA | − |
| | | | | 90941310- | 90946031- | | |
| | | | | 90972743 | 90946050 | | |
| 927 | 84146 | ZNF644 | intron_06 | chr1: | chr1: | AAUUAUAGUUAGGAUCUGUC | + |
| | | | | 90941310- | 90945974- | | |
| | | | | 90972743 | 90945993 | | |
| 928 | 84146 | ZNF644 | intron_06 | chr1: | chr1: | CCACUAGCUGAAUUAUAGUU | + |
| | | | | 90941310- | 90945964- | | |
| | | | | 90972743 | 90945983 | | |
| 929 | 84146 | ZNF644 | intron_06 | chr1: | chr1: | UAAUGAACUAAUAUACAAUG | − |
| | | | | 90941310- | 90945904- | | |
| | | | | 90972743 | 90945923 | | |
| 930 | 84146 | ZNF644 | intron_06 | chr1: | chr1: | AAUUAUUCGUGAACAGAAAG | − |
| | | | | 90941310- | 90945728- | | |
| | | | | 90972743 | 90945747 | | |
| 931 | 84146 | ZNF644 | intron_06 | chr1: | chr1: | UCCCUCUGAGCAAGCUUCCU | + |
| | | | | 90941310- | 90945649- | | |
| | | | | 90972743 | 90945668 | | |
| 932 | 84146 | ZNF644 | intron_06 | chr1: | chr1: | GACCUAGGAAGCUUGCUCAG | − |
| | | | | 90941310- | 90945654- | | |
| | | | | 90972743 | 90945673 | | |
| 933 | 84146 | ZNF644 | intron_06 | chr1: | chr1: | CCUCACUACACUACAUCUUG | + |
| | | | | 90941310- | 90945573- | | |
| | | | | 90972743 | 90945592 | | |
| 934 | 84146 | ZNF644 | intron_06 | chr1: | chr1: | CCUCAAGAUGUAGUGUAGUG | − |
| | | | | 90941310- | 90945576- | | |
| | | | | 90972743 | 90945595 | | |
| 935 | 84146 | ZNF644 | intron_06 | chr1: | chr1: | CUCAAGAUGUAGUGUAGUGA | − |
| | | | | 90941310- | 90945575- | | |
| | | | | 90972743 | 90945594 | | |
| 936 | 84146 | ZNF644 | intron_06 | chr1: | chr1: | GAGACAAUAAGUCUAGCUUG | − |
| | | | | 90941310- | 90945501- | | |
| | | | | 90972743 | 90945520 | | |
| 937 | 84146 | ZNF644 | intron_06 | chr1: | chr1: | GUAGGAACGUAUUUGAAGGU | + |
| | | | | 90941310- | 90945113- | | |
| | | | | 90972743 | 90945132 | | |
| 938 | 84146 | ZNF644 | intron_06 | chr1: | chr1: | AGUAGGAACGUAUUUGAAGG | + |
| | | | | 90941310- | 90945112- | | |
| | | | | 90972743 | 90945131 | | |
| 939 | 84146 | ZNF644 | intron_06 | chr1: | chr1: | CAAAGUAGGAACGUAUUUGA | + |
| | | | | 90941310- | 90945109- | | |
| | | | | 90972743 | 90945128 | | |
| 940 | 84146 | ZNF644 | intron_06 | chr1: | chr1: | UUAGGUUACUAUAACAAAGU | + |
| | | | | 90941310- | 90945095- | | |
| | | | | 90972743 | 90945114 | | |
| 941 | 84146 | ZNF644 | intron_06 | chr1: | chr1: | AGAGACAUCAAUACCAGUUU | + |
| | | | | 90941310- | 90945077- | | |
| | | | | 90972743 | 90945096 | | |
| 942 | 84146 | ZNF644 | intron_06 | chr1: | chr1: | UGAGAUACAAUGCAGAACCA | + |
| | | | | 90941310- | 90944874- | | |
| | | | | 90972743 | 90944893 | | |
| 943 | 84146 | ZNF644 | intron_06 | chr1: | chr1: | ACAAGUAGAUUAACGUUUUG | + |
| | | | | 90941310- | 90944744- | | |
| | | | | 90972743 | 90944763 | | |
| 944 | 84146 | ZNF644 | intron_06 | chr1: | chr1: | GACAGUAUAGAUUUGAUGAC | − |
| | | | | 90941310- | 90944640- | | |
| | | | | 90972743 | 90944659 | | |
| 945 | 84146 | ZNF644 | intron_06 | chr1: | chr1: | CACAUCCACAUAAUAAAAUA | + |
| | | | | 90941310- | 90944475- | | |
| | | | | 90972743 | 90944494 | | |
| 946 | 84146 | ZNF644 | intron_06 | chr1: | chr1: | UCUAUGUUUUUGAACAAAAC | − |
| | | | | 90941310- | 90944341- | | |
| | | | | 90972743 | 90944360 | | |
| 947 | 84146 | ZNF644 | intron_06 | chr1: | chr1: | CUUUAUAAGAGCUUUUCAUA | + |
| | | | | 90941310- | 90944295- | | |
| | | | | 90972743 | 90944314 | | |
| 948 | 84146 | ZNF644 | intron_06 | chr1: | chr1: | AAGCUCUUAUAAAGCCAUUC | − |
| | | | | 90941310- | 90944289- | | |
| | | | | 90972743 | 90944308 | | |
| 949 | 84146 | ZNF644 | intron_06 | chr1: | chr1: | CUGCAUCCUCAUUACCGGAA | + |
| | | | | 90941310- | 90944272- | | |
| | | | | 90972743 | 90944291 | | |
| 950 | 84146 | ZNF644 | intron_06 | chr1: | chr1: | AUAAAGCCAUUCCGGUAAUG | − |
| | | | | 90941310- | 90944281- | | |
| | | | | 90972743 | 90944300 | | |
| 951 | 84146 | ZNF644 | intron_06 | chr1: | chr1: | UUGGUACCCCCUGAACAGCA | + |
| | | | | 90941310- | 90944214- | | |
| | | | | 90972743 | 90944233 | | |
| 952 | 84146 | ZNF644 | intron_06 | chr1: | chr1: | AUUGGUACCCCCUGAACAGC | + |
| | | | | 90941310- | 90944213- | | |
| | | | | 90972743 | 90944232 | | |
| 953 | 84146 | ZNF644 | intron_06 | chr1: | chr1: | AGUUUGUCACCCUGCUGUUC | − |
| | | | | 90941310- | 90944226- | | |
| | | | | 90972743 | 90944245 | | |
| 954 | 84146 | ZNF644 | intron_06 | chr1: | chr1: | GUUUGUCACCCUGCUGUUCA | − |
| | | | | 90941310- | 90944225- | | |
| | | | | 90972743 | 90944244 | | |
| 955 | 84146 | ZNF644 | intron_06 | chr1: | chr1: | UUUGUCACCCUGCUGUUCAG | − |
| | | | | 90941310- | 90944224- | | |
| | | | | 90972743 | 90944243 | | |
| 956 | 84146 | ZNF644 | intron_06 | chr1: | chr1: | UUGUCACCCUGCUGUUCAGG | − |
| | | | | 90941310- | 90944223- | | |
| | | | | 90972743 | 90944242 | | |
| 957 | 84146 | ZNF644 | intron_06 | chr1: | chr1: | CCCAUUUAAAGCAACUUCAU | + |
| | | | | 90941310- | 90944195- | | |
| | | | | 90972743 | 90944214 | | |
| 958 | 84146 | ZNF644 | intron_06 | chr1: | chr1: | CCAAUGAAGUUGCUUUAAAU | − |
| | | | | 90941310- | 90944198- | | |
| | | | | 90972743 | 90944217 | | |
| 959 | 84146 | ZNF644 | intron_06 | chr1: | chr1: | ACCUCUAACACCAGAACAGA | − |
| | | | | 90941310- | 90944154- | | |
| | | | | 90972743 | 90944173 | | |
| 960 | 84146 | ZNF644 | intron_06 | chr1: | chr1: | GGUGGAACAUAGAACAUGGA | − |
| | | | | 90941310- | 90944132- | | |
| | | | | 90972743 | 90944151 | | |
| 961 | 84146 | ZNF644 | intron_06 | chr1: | chr1: | CAUGGAAGGUUUUACCUCUG | − |
| | | | | 90941310- | 90944118- | | |
| | | | | 90972743 | 90944137 | | |
| 962 | 84146 | ZNF644 | intron_06 | chr1: | chr1: | CUUCACUGUUACCACCUCAG | + |
| | | | | 90941310- | 90944101- | | |
| | | | | 90972743 | 90944120 | | |
| 963 | 84146 | ZNF644 | intron_06 | chr1: | chr1: | CCCACUUAUUAUAAUCACAG | + |
| | | | | 90941310- | 90943978- | | |
| | | | | 90972743 | 90943997 | | |
| 964 | 84146 | ZNF644 | intron_06 | chr1: | chr1: | ACCUCUGUGAUUAUAAUAAG | − |
| | | | | 90941310- | 90943982- | | |
| | | | | 90972743 | 90944001 | | |
| 965 | 84146 | ZNF644 | intron_06 | chr1: | chr1: | CCUCUGUGAUUAUAAUAAGU | − |
| | | | | 90941310- | 90943981- | | |
| | | | | 90972743 | 90944000 | | |
| 966 | 84146 | ZNF644 | intron_06 | chr1: | chr1: | UGAAUGGUAGUGUAGCAUUA | − |
| | | | | 90941310- | 90943838- | | |
| | | | | 90972743 | 90943857 | | |
| 967 | 84146 | ZNF644 | intron_06 | chr1: | chr1: | GAAAGGGUGUGCAAAUGUCA | + |
| | | | | 90941310- | 90943798- | | |
| | | | | 90972743 | 90943817 | | |
| 968 | 84146 | ZNF644 | intron_06 | chr1: | chr1: | GCAGCACAUAAACAUGUGUU | + |
| | | | | 90941310- | 90943776- | | |
| | | | | 90972743 | 90943795 | | |
| 969 | 84146 | ZNF644 | intron_06 | chr1: | chr1: | UGUGAUGUAGAGACUCCUGC | − |
| | | | | 90941310- | 90943736- | | |
| | | | | 90972743 | 90943755 | | |
| 970 | 84146 | ZNF644 | intron_06 | chr1: | chr1: | GUGAUGUAGAGACUCCUGCU | − |
| | | | | 90941310- | 90943735- | | |
| | | | | 90972743 | 90943754 | | |
| 971 | 84146 | ZNF644 | intron_06 | chr1: | chr1: | GUGCUAAUGCAUCACCCAGC | + |
| | | | | 90941310- | 90943718- | | |
| | | | | 90972743 | 90943737 | | |
| 972 | 84146 | ZNF644 | intron_06 | chr1: | chr1: | UUAAUGAUAUCACUCCAAAA | + |
| | | | | 90941310- | 90943696- | | |
| | | | | 90972743 | 90943715 | | |
| 973 | 84146 | ZNF644 | intron_06 | chr1: | chr1: | ACAGAAUUCUAAAACCUACG | − |
| | | | | 90941310- | 90943621- | | |
| | | | | 90972743 | 90943640 | | |
| 974 | 84146 | ZNF644 | intron_06 | chr1: | chr1: | CAGAAUUCUAAAACCUACGA | − |
| | | | | 90941310- | 90943620- | | |
| | | | | 90972743 | 90943639 | | |
| 975 | 84146 | ZNF644 | intron_06 | chr1: | chr1: | UCAUUUUUUCCACCCCUCGU | + |
| | | | | 90941310- | 90943604- | | |
| | | | | 90972743 | 90943623 | | |
| 976 | 84146 | ZNF644 | intron_06 | chr1: | chr1: | AUUCUAAAACCUACGAGGGG | − |
| | | | | 90941310- | 90943616- | | |
| | | | | 90972743 | 90943635 | | |
| 977 | 84146 | ZNF644 | intron_06 | chr1: | chr1: | GGGGUGGAAAAAAUGAUUGU | − |
| | | | | 90941310- | 90943600- | | |
| | | | | 90972743 | 90943619 | | |
| 978 | 84146 | ZNF644 | intron_06 | chr1: | chr1: | AUGUCAAAGAUACUGGUAAU | − |
| | | | | 90941310- | 90943566- | | |
| | | | | 90972743 | 90943585 | | |
| 979 | 84146 | ZNF644 | intron_06 | chr1: | chr1: | AUAUUUCUACUAUCCUUUAA | + |
| | | | | 90941310- | 90943077- | | |
| | | | | 90972743 | 90943096 | | |
| 980 | 84146 | ZNF644 | intron_06 | chr1: | chr1: | AAUAUUUCUACUAUCCUUUA | + |
| | | | | 90941310- | 90943076- | | |
| | | | | 90972743 | 90943095 | | |
| 981 | 84146 | ZNF644 | intron_06 | chr1: | chr1: | AAUGGUAAACAAGACAAGUU | + |
| | | | | 90941310- | 90942913- | | |
| | | | | 90972743 | 90942932 | | |
| 982 | 84146 | ZNF644 | intron_06 | chr1: | chr1: | UACUUCAAUUAGUUAAUACC | − |
| | | | | 90941310- | 90942789- | | |
| | | | | 90972743 | 90942808 | | |
| 983 | 84146 | ZNF644 | intron_06 | chr1: | chr1: | CUUCAAUUAGUUAAUACCUG | − |
| | | | | 90941310- | 90942787- | | |
| | | | | 90972743 | 90942806 | | |
| 984 | 84146 | ZNF644 | intron_06 | chr1: | chr1: | AAUUUAAAGAACCUGCUAUU | + |
| | | | | 90941310- | 90942758- | | |
| | | | | 90972743 | 90942777 | | |
| 985 | 84146 | ZNF644 | intron_06 | chr1: | chr1: | AUUCAAACACAAGUUCCAUC | + |
| | | | | 90941310- | 90942672- | | |
| | | | | 90972743 | 90942691 | | |
| 986 | 84146 | ZNF644 | intron_06 | chr1: | chr1: | UGCAAGGGAACUUUUCAUAG | − |
| | | | | 90941310- | 90942378- | | |
| | | | | 90972743 | 90942397 | | |
| 987 | 84146 | ZNF644 | intron_06 | chr1: | chr1: | GCAAGGGAACUUUUCAUAGU | − |
| | | | | 90941310- | 90942377- | | |
| | | | | 90972743 | 90942396 | | |
| 988 | 84146 | ZNF644 | intron_06 | chr1: | chr1: | GGGGGUAGGCAUUACAGUCA | − |
| | | | | 90941310- | 90942357- | | |
| | | | | 90972743 | 90942376 | | |
| 989 | 84146 | ZNF644 | intron_06 | chr1: | chr1: | CAUUACAGUCAUGGUGUGAA | − |
| | | | | 90941310- | 90942348- | | |
| | | | | 90972743 | 90942367 | | |
| 990 | 84146 | ZNF644 | intron_06 | chr1: | chr1: | GAUCUGGUGGUCUAGUGCAA | + |
| | | | | 90941310- | 90942160- | | |
| | | | | 90972743 | 90942179 | | |
| 991 | 84146 | ZNF644 | intron_06 | chr1: | chr1: | UUAUCCCACUUAUGAUCUGG | + |
| | | | | 90941310- | 90942147- | | |
| | | | | 90972743 | 90942166 | | |
| 992 | 84146 | ZNF644 | intron_06 | chr1: | chr1: | CAGUUAUCCCACUUAUGAUC | + |
| | | | | 90941310- | 90942144- | | |
| | | | | 90972743 | 90942163 | | |
| 993 | 84146 | ZNF644 | intron_06 | chr1: | chr1: | UAGACCACCAGAUCAUAAGU | − |
| | | | | 90941310- | 90942154- | | |
| | | | | 90972743 | 90942173 | | |
| 994 | 84146 | ZNF644 | intron_06 | chr1: | chr1: | UAGAGAAUGCCUAGAAUUUG | − |
| | | | | 90941310- | 90942120- | | |
| | | | | 90972743 | 90942139 | | |
| 995 | 84146 | ZNF644 | intron_06 | chr1: | chr1: | AGUUAAGGUUAAUUUGAAUA | − |
| | | | | 90941310- | 90941993- | | |
| | | | | 90972743 | 90942012 | | |
| 996 | 84146 | ZNF644 | intron_06 | chr1: | chr1: | CACUUAAGUAGGUUUAUCAG | + |
| | | | | 90941310- | 90941707- | | |
| | | | | 90972743 | 90941726 | | |
| 997 | 84146 | ZNF644 | intron_06 | chr1: | chr1: | GUAUUAGCAAUCUCCACGUG | − |
| | | | | 90941310- | 90941673- | | |
| | | | | 90972743 | 90941692 | | |
| 998 | 84146 | ZNF644 | intron_06 | chr1: | chr1: | AUUUUGUAUACAUGACGUAU | − |
| | | | | 90941310- | 90941532- | | |
| | | | | 90972743 | 90941551 | | |
| 999 | 84146 | ZNF644 | intron_06 | chr1: | chr1: | AAGAACAAUAUUAACCUUUG | − |
| | | | | 90941310- | 90941487- | | |
| | | | | 90972743 | 90941506 | | |
| 1000 | 84146 | ZNF644 | exon_07_c.1/ | chr1: | chr1: | CUAGACUAAAUGUGUUAAAU | − |
| | | | nc.1 | 90940949- | 90941294- | | |
| | | | | 90941309 | 90941313 | | |
| 1001 | 84146 | ZNF644 | exon_07_c.1/ | chr1: | chr1: | CUUCAAAUCAUCCAUAUUGU | + |
| | | | nc.1 | 90940949- | 90941264- | | |
| | | | | 90941309 | 90941283 | | |
| 1002 | 84146 | ZNF644 | exon_07_c.1/ | chr1: | chr1: | UUCUUUAGCACCAGUAAUAU | + |
| | | | nc.1 | 90940949- | 90941234- | | |
| | | | | 90941309 | 90941253 | | |
| 1003 | 84146 | ZNF644 | exon_07_c.1/ | chr1: | chr1: | AAGAUAAACACCGAUAUUAC | − |
| | | | nc.1 | 90940949- | 90941247- | | |
| | | | | 90941309 | 90941266 | | |
| 1004 | 84146 | ZNF644 | exon_07_c.1/ | chr1: | chr1: | UUGUCCUUUGACAGUUCUUC | + |
| | | | nc.1 | 90940949- | 90941109- | | |
| | | | | 90941309 | 90941128 | | |
| 1005 | 84146 | ZNF644 | exon_07_c.1/ | chr1: | chr1: | UCUGAAAACGCCUUAAGUGG | − |
| | | | nc.1 | 90940949- | 90941088- | | |
| | | | | 90941309 | 90941107 | | |
| 1006 | 84146 | ZNF644 | exon_07_c.1/ | chr1: | chr1: | UCUAGUCUAUUUAUACAUGC | − |
| | | | nc.1 | 90940949- | 90941061- | | |
| | | | | 90941309 | 90941080 | | |
| 1007 | 84146 | ZNF644 | exon_07_c.1/ | chr1: | chr1: | GAAAACUUUAUCUUGCCUAA | − |
| | | | nc.1 | 90940949- | 90941019- | | |
| | | | | 90941309 | 90941038 | | |
| 1008 | 84146 | ZNF644 | exon_07_c.1/ | chr1: | chr1: | AUUGGAAGUCUUAGUUAAGG | + |
| | | | nc.1 | 90940949- | 90940964- | | |
| | | | | 90941309 | 90940983 | | |
| 1009 | 84146 | ZNF644 | exon_07_c.1/ | chr1: | chr1: | AACACUGCCUUUAUUCAUAU | + |
| | | | nc.1 | 90940949- | 90940946- | | |
| | | | | 90941309 | 90940965 | | |
| 1010 | 84146 | ZNF644 | exon_07_c.1/ | chr1: | chr1: | GGCAGUGUUUCAUUAACCAC | − |
| | | | | 90940121- | 90940935- | | |
| | | | | 90940948 | 90940954 | | |
| 1011 | 84146 | ZNF644 | exon_07_c.1/ | chr1: | chr1: | AUUAACCACUGGACAGCCUG | − |
| | | | | 90940121- | 90940924- | | |
| | | | | 90940948 | 90940943 | | |
| 1012 | 84146 | ZNF644 | exon_07_c.1/ | chr1: | chr1: | UCUGUUGUUGGCUGAUCCAC | + |
| | | | | 90940121- | 90940905- | | |
| | | | | 90940948 | 90940924 | | |
| 1013 | 84146 | ZNF644 | exon_07_c.1/ | chr1: | chr1: | AGUUUUUUAUUGGAAUGGGU | + |
| | | | | 90940121- | 90940773- | | |
| | | | | 90940948 | 90940792 | | |
| 1014 | 84146 | ZNF644 | exon_07_c.1/ | chr1: | chr1: | UAGUUUUUUAUUGGAAUGGG | + |
| | | | | 90940121- | 90940772- | | |
| | | | | 90940948 | 90940791 | | |
| 1015 | 84146 | ZNF644 | exon_07_c.1/ | chr1: | chr1: | AGAGGUAGGUAGUUUUUUAU | + |
| | | | | 90940121- | 90940763- | | |
| | | | | 90940948 | 90940782 | | |
| 1016 | 84146 | ZNF644 | exon_07_c.1/ | chr1: | chr1: | GUCACAACCAACUGAAGCAG | + |
| | | | | 90940121- | 90940745- | | |
| | | | | 90940948 | 90940764 | | |
| 1017 | 84146 | ZNF644 | exon_07_c.1/ | chr1: | chr1: | UUUAAUAAAUCAAGUAGAGG | − |
| | | | | 90940121- | 90940684- | | |
| | | | | 90940948 | 90940703 | | |
| 1018 | 84146 | ZNF644 | exon_07_c.1/ | chr1: | chr1: | UUAAUAAAUCAAGUAGAGGU | − |
| | | | | 90940121- | 90940683- | | |
| | | | | 90940948 | 90940702 | | |
| 1019 | 84146 | ZNF644 | exon_07_c.1/ | chr1: | chr1: | GAUUGUGUCAAUACAGUAAC | − |
| | | | | 90940121- | 90940629- | | |
| | | | | 90940948 | 90940648 | | |
| 1020 | 84146 | ZNF644 | exon_07_c.1/ | chr1: | chr1: | CCUAAAUCCAUCUGUACCUG | + |
| | | | | 90940121- | 90940598- | | |
| | | | | 90940948 | 90940617 | | |
| 1021 | 84146 | ZNF644 | exon_07_c.1/ | chr1: | chr1: | UCAAUUUCUUAUGACUAAUG | − |
| | | | | 90940121- | 90940537- | | |
| | | | | 90940948 | 90940556 | | |
| 1022 | 84146 | ZNF644 | exon_07_c.1/ | chr1: | chr1: | UCUAGACCUAUUUUAGAAUG | + |
| | | | | 90940121- | 90940491- | | |
| | | | | 90940948 | 90940510 | | |
| 1023 | 84146 | ZNF644 | exon_07_c.1/ | chr1: | chr1: | GCUCCACCUCAUUCUAAAAU | − |
| | | | | 90940121- | 90940500- | | |
| | | | | 90940948 | 90940519 | | |
| 1024 | 84146 | ZNF644 | exon_07_c.1/ | chr1: | chr1: | CAAGAUAACUCGUUAUACCG | − |
| | | | | 90940121- | 90940441- | | |
| | | | | 90940948 | 90940460 | | |
| 1025 | 84146 | ZNF644 | exon_07_c.1/ | chr1: | chr1: | GUUAUAAAUUAGAUGUUGGA | + |
| | | | | 90940121- | 90940256- | | |
| | | | | 90940948 | 90940275 | | |
| 1026 | 84146 | ZNF644 | exon_07_c.1/ | chr1: | chr1: | UCUCUUCUCCACACUUAUCU | + |
| | | | | 90940121- | 90940234- | | |
| | | | | 90940948 | 90940253 | | |
| 1027 | 84146 | ZNF644 | exon_07_c.1/ | chr1: | chr1: | CUCUCUUCUCCACACUUAUC | + |
| | | | | 90940121- | 90940233- | | |
| | | | | 90940948 | 90940252 | | |
| 1028 | 84146 | ZNF644 | exon_07_c.1/ | chr1: | chr1: | UCUUCGGUACUGAAAGUAGC | + |
| | | | | 90940121- | 90940137- | | |
| | | | | 90940948 | 90940156 | | |
| 1029 | 84146 | ZNF644 | exon_07_c.1/ | chr1: | chr1: | UUUGUACAGGGGUAGAAUGA | + |
| | | | | 90940121- | 90940113- | | |
| | | | | 90940948 | 90940132 | | |
| 1030 | 84146 | ZNF644 | exon_07_c.1/ | chr1: | chr1: | AUUCACAUUGCACUUUGUAC | + |
| | | | nc.2 | 90940070- | 90940100- | | |
| | | | | 90940120 | 90940119 | | |
| 1031 | 84146 | ZNF644 | exon_07_c.1/ | chr1: | chr1: | CAAAGUGCAAUGUGAAUUUU | − |
| | | | nc.2 | 90940070- | 90940097- | | |
| | | | | 90940120 | 90940116 | | |
| 1032 | 84146 | ZNF644 | exon_07_c.1/ | chr1: | chr1: | AAUGAUACAUCAUAUGCCUG | + |
| | | | nc.2 | 90940070- | 90940057- | | |
| | | | | 90940120 | 90940076 | | |
| 1033 | 84146 | ZNF644 | exon_07_c.2/ | chr1: | chr1: | CAUAUGAUGUAUCAUUUAGA | − |
| | | | | 90938272- | 90940053- | | |
| | | | | 90940069 | 90940072 | | |
| 1034 | 84146 | ZNF644 | exon_07_c.2/ | chr1: | chr1: | AUGGCCUUGGGACAUUAAGA | + |
| | | | | 90938272- | 90940012- | | |
| | | | | 90940069 | 90940031 | | |
| 1035 | 84146 | ZNF644 | exon _07_c.2/ | chr1: | chr1: | UUCGCCAUCUUAAUGUCCCA | − |
| | | | | 90938272- | 90940019- | | |
| | | | | 90940069 | 90940038 | | |
| 1036 | 84146 | ZNF644 | exon_07_c.2/ | chr1: | chr1: | UCUCUACAAGCAUAUGGCCU | + |
| | | | | 90938272- | 90939999- | | |
| | | | | 90940069 | 90940018 | | |
| 1037 | 84146 | ZNF644 | exon_07_c.2/ | chr1: | chr1: | UCGUGAAUUGAAAGAACUUC | − |
| | | | | 90938272- | 90939892- | | |
| | | | | 90940069 | 90939911 | | |
| 1038 | 84146 | ZNF644 | exon_07_c.2/ | chr1: | chr1: | CCCCAUGUGCAAUGCCUCGA | + |
| | | | | 90938272- | 90939706- | | |
| | | | | 90940069 | 90939725 | | |
| 1039 | 84146 | ZNF644 | exo 07_c.2/ | chr1: | chr1: | GCCAUCGAGGCAUUGCACAU | − |
| | | | | 90938272- | 90939710- | | |
| | | | | 90940069 | 90939729 | | |
| 1040 | 84146 | ZNF644 | exon_07_c.2/ | chr1: | chr1: | CCAUCGAGGCAUUGCACAUG | − |
| | | | | 90938272- | 90939709- | | |
| | | | | 90940069 | 90939728 | | |
| 1041 | 84146 | ZNF644 | exon_07_c.2/ | chr1: | chr1: | AGGCAUUGCACAUGGGGCAG | − |
| | | | | 90938272- | 90939703- | | |
| | | | | 90940069 | 90939722 | | |
| 1042 | 84146 | ZNF644 | exon_07_c.2/ | chr1: | chr1: | GGCAGUGGUAAAAUGCCCUA | − |
| | | | | 90938272- | 90939688- | | |
| | | | | 90940069 | 90939707 | | |
| 1043 | 84146 | ZNF644 | exon_07_c.2/ | chr1: | chr1: | GCAAUAUCAGAAGUGACCAU | + |
| | | | | 90938272- | 90939669- | | |
| | | | | 90940069 | 90939688 | | |
| 1044 | 84146 | ZNF644 | exon_07_c.2/ | chr1: | chr1: | CUUUUGGCUGAAGUAGUAAA | + |
| | | | | 90938272- | 90939558- | | |
| | | | | 90940069 | 90939577 | | |
| 1045 | 84146 | ZNF644 | exon_07_c.2/ | chr1: | chr1: | CACUAUCUACAGGUUCUUCA | + |
| | | | | 90938272- | 90939442- | | |
| | | | | 90940069 | 90939461 | | |
| 1046 | 84146 | ZNF644 | exon_07_c.2/ | chr1: | chr1: | UUAGUGCUAUCACUAUCUAC | + |
| | | | | 90938272- | 90939432- | | |
| | | | | 90940069 | 90939451 | | |
| 1047 | 84146 | ZNF644 | exon_07_c.2/ | chr1: | chr1: | CGAGCUUUCUGUAUUCUGUG | + |
| | | | | 90938272- | 90939291- | | |
| | | | | 90940069 | 90939310 | | |
| 1048 | 84146 | ZNF644 | exon_07_c.2/ | chr1: | chr1: | CACACAGAAUACAGAAAGCU | − |
| | | | | 90938272- | 90939293- | | |
| | | | | 90940069 | 90939312 | | |
| 1049 | 84146 | ZNF644 | exon_07_c.2/ | chr1: | chr1: | CGGAAAAGCAUUGCCCAAUC | − |
| | | | | 90938272- | 90939273- | | |
| | | | | 90940069 | 90939292 | | |
| 1050 | 84146 | ZNF644 | exon_07_c.2/ | chr1: | chr1: | GCACAUGUUUACACCUGAUU | + |
| | | | | 90938272- | 90939257- | | |
| | | | | 90940069 | 90939276 | | |
| 1051 | 84146 | ZNF644 | exon_07_c.2/ | chr1: | chr1: | UGCACAUGUUUACACCUGAU | + |
| | | | | 90938272- | 90939256- | | |
| | | | | 90940069 | 90939275 | | |
| 1052 | 84146 | ZNF644 | exon_07_c.2/ | chr1: | chr1: | GAUGGAAAUACUUAGGUUUU | + |
| | | | | 90938272- | 90939184- | | |
| | | | | 90940069 | 90939203 | | |
| 1053 | 84146 | ZNF644 | exon_07_c.2/ | chr1: | chr1: | CUUUUGGCGUCAGGCUUAUG | + |
| | | | | 90938272- | 90938967- | | |
| | | | | 90940069 | 90938986 | | |
| 1054 | 84146 | ZNF644 | exon_07_c.2/ | chr1: | chr1: | CUUUCAGGCCUUUUGGCGUC | + |
| | | | | 90938272- | 90938958- | | |
| | | | | 90940069 | 90938977 | | |
| 1055 | 84146 | ZNF644 | exon_07_c.2/ | chr1: | chr1: | CUCAUAAGCCUGACGCCAAA | − |
| | | | | 90938272- | 90938969- | | |
| | | | | 90940069 | 90938988 | | |
| 1056 | 84146 | ZNF644 | exon_07_c.2/ | chr1: | chr1: | AGUAAUUAAGGAAUCUAAGA | − |
| | | | | 90938272- | 90938905- | | |
| | | | | 90940069 | 90938924 | | |
| 1057 | 84146 | ZNF644 | exon_07_c.2/ | chr1: | chr1: | UCUAAGAAGGAAAGUUCUGU | − |
| | | | | 90938272- | 90938892- | | |
| | | | | 90940069 | 90938911 | | |
| 1058 | 84146 | ZNF644 | exon_07_c.2/ | chr1: | chr1: | AAGAAGGAAAGUUCUGUUGG | − |
| | | | | 90938272- | 90938889- | | |
| | | | | 90940069 | 90938908 | | |
| 1059 | 84146 | ZNF644 | exon_07_c.2/ | chr1: | chr1: | UCCUGGGAUAAUGUUGAGUU | − |
| | | | | 90938272- | 90938757- | | |
| | | | | 90940069 | 90938776 | | |
| 1060 | 84146 | ZNF644 | exon_07_c.2/ | chr1: | chr1: | GUUAGGAGACUACACUACAC | − |
| | | | | 90938272- | 90938740- | | |
| | | | | 90940069 | 90938759 | | |
| 1061 | 84146 | ZNF644 | exon_07_c.2/ | chr1: | chr1: | UUAUUUAAGAGCAAAGUGGA | − |
| | | | | 90938272- | 90938661- | | |
| | | | | 90940069 | 90938680 | | |
| 1062 | 84146 | ZNF644 | exon_07_c.2/ | chr1: | chr1: | CUUAGUUAUGACCAAAACGA | − |
| | | | | 90938272- | 90938613- | | |
| | | | | 90940069 | 90938632 | | |
| 1063 | 84146 | ZNF644 | exon_07_c.2/ | chr1: | chr1: | UUUGAAUACUAUGAAGAUAC | − |
| | | | | 90938272- | 90938583- | | |
| | | | | 90940069 | 90938602 | | |
| 1064 | 84146 | ZNF644 | exon_07_c.2/ | chr1: | chr1: | UCUGCAGUUUCUAAAUGCUG | + |
| | | | | 90938272- | 90938526- | | |
| | | | | 90940069 | 90938545 | | |
| 1065 | 84146 | ZNF644 | exon_07_c.2/ | chr1: | chr1: | CACCUGUUUCAAAUGUUGCU | + |
| | | | | 90938272- | 90938428- | | |
| | | | | 90940069 | 90938447 | | |
| 1066 | 84146 | ZNF644 | exon_07_c.2/ | chr1: | chr1: | UGCCCAGCAACAUUUGAAAC | − |
| | | | | 90938272- | 90938433- | | |
| | | | | 90940069 | 90938452 | | |
| 1067 | 84146 | ZNF644 | exon_07_c.2/ | chr1: | chr1: | GCAACAUUUGAAACAGGUGU | − |
| | | | | 90938272- | 90938427- | | |
| | | | | 90940069 | 90938446 | | |
| 1068 | 84146 | ZNF644 | exon_07_c.2/ | chr1: | chr1: | UUGGGUUAUCAAAUCAUGUC | − |
| | | | | 90938272- | 90938408- | | |
| | | | | 90940069 | 90938427 | | |
| 1069 | 84146 | ZNF644 | exon_07_c.2/ | chr1: | chr1: | UGGGUUAUCAAAUCAUGUCA | − |
| | | | | 90938272- | 90938407- | | |
| | | | | 90940069 | 90938426 | | |
| 1070 | 84146 | ZNF644 | exon_07_c.2/ | chr1: | chr1: | GGUUAUCAAAUCAUGUCAGG | − |
| | | | | 90938272- | 90938405- | | |
| | | | | 90940069 | 90938424 | | |
| 1071 | 84146 | ZNF644 | exon_07_c.2/ | chr1: | chr1: | UUCUGGUGAUACAACAUGAC | + |
| | | | | 90938272- | 90938348- | | |
| | | | | 90940069 | 90938367 | | |
| 1072 | 84146 | ZNF644 | exon_07_c.2/ | chr1: | chr1: | UCACUUGUGGCUAUUUGUUC | + |
| | | | | 90938272- | 90938331- | | |
| | | | | 90940069 | 90938350 | | |
| 1073 | 84146 | ZNF644 | exon_07_c.2/ | chr1: | chr1: | CCUUUUCUAACUCGUUUAAC | + |
| | | | | 90938272- | 90938271- | | |
| | | | | 90940069 | 90938290 | | |
| 1074 | 84146 | ZNF644 | exon_07_c.2/ | chr1: | chr1: | CCUGUUAAACGAGUUAGAAA | − |
| | | | | 90938272- | 90938274- | | |
| | | | | 90940069 | 90938293 | | |
| 1075 | 84146 | ZNF644 | intron_07 | chr1: | chr1: | UAGAAAAGGUAAGUUCUCCA | − |
| | | | | 90938091- | 90938260- | | |
| | | | | 90938271 | 90938279 | | |
| 1076 | 84146 | ZNF644 | intron_07 | chr1: | chr1: | AGAAAAGGUAAGUUCUCCAU | − |
| | | | | 90938091- | 90938259- | | |
| | | | | 90938271 | 90938278 | | |
| 1077 | 84146 | ZNF644 | exon_08_c | chr1: | chr1: | CUGACAAGUGUGUUCAGAAG | + |
| | | | | 90937939- | 90938050- | | |
| | | | | 90938090 | 90938069 | | |
| 1078 | 84146 | ZNF644 | exon_08_c | chr1: | chr1: | GAACACACUUGUCAGCUCUG | − |
| | | | | 90937939- | 90938045- | | |
| | | | | 90938090 | 90938064 | | |
| 1079 | 84146 | ZNF644 | exon_08_c | chr1: | chr1: | GGUUGGUUUGAUACUAAAAU | − |
| | | | | 90937939- | 90938021- | | |
| | | | | 90938090 | 90938040 | | |
| 1080 | 84146 | ZNF644 | exon_08_c | chr1: | chr1: | AAAGACUUGGAAAGACGAAA | − |
| | | | | 90937939- | 90937969- | | |
| | | | | 90938090 | 90937988 | | |
| 1081 | 84146 | ZNF644 | exon_08_c | chr1: | chr1: | AAGACUUGGAAAGACGAAAU | − |
| | | | | 90937939- | 90937968- | | |
| | | | | 90938090 | 90937987 | | |
| 1082 | 84146 | ZNF644 | exon_08_c | chr1: | chr1: | AUCUCAUUCAGAACACAGAU | + |
| | | | | 90937939- | 90937928- | | |
| | | | | 90938090 | 90937947 | | |
| 1083 | 84146 | ZNF644 | exon_08_c | chr1: | chr1: | UUUUUGAGCUACAAAUGGUC | + |
| | | | | 90937485- | 90937843- | | |
| | | | | 90937882 | 90937862 | | |
| 1084 | 84146 | ZNF644 | exon_08_c | chr1: | chr1: | CCAUUACGGUAUGCUUCAAG | + |
| | | | | 90937485- | 90937781- | | |
| | | | | 90937882 | 90937800 | | |
| 1085 | 84146 | ZNF644 | exon_08_c | chr1: | chr1: | CCUCUUGAAGCAUACCGUAA | − |
| | | | | 90937485- | 90937784- | | |
| | | | | 90937882 | 90937803 | | |
| 1086 | 84146 | ZNF644 | exon_08_c | chr1: | chr1: | CUUCAGUCUUUAGGCCAUUA | + |
| | | | | 90937485- | 90937767- | | |
| | | | | 90937882 | 90937786 | | |
| 1087 | 84146 | ZNF644 | exon_08_c | chr1: | chr1: | GACUGAUUCUUUUUCCCACU | + |
| | | | | 90937485- | 90937670- | | |
| | | | | 90937882 | 90937689 | | |
| 1088 | 84146 | ZNF644 | exon_08_c | chr1: | chr1: | GUCUGAUUAUGGAUCUUUUG | + |
| | | | | 90937485- | 90937583- | | |
| | | | | 90937882 | 90937602 | | |
| 1089 | 84146 | ZNF644 | exon_08_c | chr1: | chr1: | AACGGACUAUCCUCAUUUAA | + |
| | | | | 90937485- | 90937526- | | |
| | | | | 90937882 | 90937545 | | |
| 1090 | 84146 | ZNF644 | exon_08_c | chr1: | chr1: | UUUUGUGGCUGAUACAUCAA | + |
| | | | | 90937485- | 90937508- | | |
| | | | | 90937882 | 90937527 | | |
| 1091 | 84146 | ZNF644 | intron_08 | chr1: | chr1: | UUCAAUUAUGCCUUCUUUAA | − |
| | | | | 90935529- | 90937421- | | |
| | | | | 90937484 | 90937440 | | |
| 1092 | 84146 | ZNF644 | intron_08 | chr1: | chr1: | CUUCUUUAAUGGAAUCUAUG | − |
| | | | | 90935529- | 90937410- | | |
| | | | | 90937484 | 90937429 | | |
| 1093 | 84146 | ZNF644 | intron_08 | chr1: | chr1: | UACAUUCAGACCAAGAAAAG | + |
| | | | | 90935529- | 90937259- | | |
| | | | | 90937484 | 90937278 | | |
| 1094 | 84146 | ZNF644 | intron_08 | chr1: | chr1: | CUUUUCUUGGUCUGAAUGUA | − |
| | | | | 90935529- | 90937259- | | |
| | | | | 90937484 | 90937278 | | |
| 1095 | 84146 | ZNF644 | intron_08 | chr1: | chr1: | GGAGGAUGGUUGUGGAUGGG | − |
| | | | | 90935529- | 90937147- | | |
| | | | | 90937484 | 90937166 | | |
| 1096 | 84146 | ZNF644 | intron_08 | chr1: | chr1: | AACUGGCAAAAAUAUGACAG | + |
| | | | | 90935529- | 90937084- | | |
| | | | | 90937484 | 90937103 | | |
| 1097 | 84146 | ZNF644 | intron_08 | chr1: | chr1: | ACUCUUAAAGCUGAAUGGAU | − |
| | | | | 90935529- | 90936997- | | |
| | | | | 90937484 | 90937016 | | |
| 1098 | 84146 | ZNF644 | intron_08 | chr1: | chr1: | UGGAGAGGGAGAUGUUUCUU | + |
| | | | | 90935529- | 90936954- | | |
| | | | | 90937484 | 90936973 | | |
| 1099 | 84146 | ZNF644 | intron_08 | chr1: | chr1: | UCCGCAAUACAAUGAGGAAU | + |
| | | | | 90935529- | 90936934- | | |
| | | | | 90937484 | 90936953 | | |
| 1100 | 84146 | ZNF644 | intron_08 | chr1: | chr1: | AACUAUUCCGCAAUACAAUG | + |
| | | | | 90935529- | 90936928- | | |
| | | | | 90937484 | 90936947 | | |
| 1101 | 84146 | ZNF644 | intron_08 | chr1: | chr1: | UCCAAUUCCUCAUUGUAUUG | − |
| | | | | 90935529- | 90936938- | | |
| | | | | 90937484 | 90936957 | | |
| 1102 | 84146 | ZNF644 | intron_08 | chr1: | chr1: | CAUUGUAUUGCGGAAUAGUU | − |
| | | | | 90935529- | 90936928- | | |
| | | | | 90937484 | 90936947 | | |
| 1103 | 84146 | ZNF644 | intron_08 | chr1: | chr1: | AUUGCGGAAUAGUUUGGUUU | − |
| | | | | 90935529- | 90936922- | | |
| | | | | 90937484 | 90936941 | | |
| 1104 | 84146 | ZNF644 | intron_08 | chr1: | chr1: | GUUUUAAGAUAGUGCUCUAA | − |
| | | | | 90935529- | 90936857- | | |
| | | | | 90937484 | 90936876 | | |
| 1105 | 84146 | ZNF644 | intron_08 | chr1: | chr1: | CAGAUGAUUUAGCUUAGUGC | − |
| | | | | 90935529- | 90936827- | | |
| | | | | 90937484 | 90936846 | | |
| 1106 | 84146 | ZNF644 | intron_08 | chr1: | chr1: | GUAUGUAUUGGCAAAUAAUG | |
| | | | | 90935529- | 90936767- | | |
| | | | | 90937484 | 90936786 | | |
| 1107 | 84146 | ZNF644 | intron_08 | chr1: | chr1: | UGGCAAUACUGAAUAAAGUU | + |
| | | | | 90935529- | 90936729- | | |
| | | | | 90937484 | 90936748 | | |
| 1108 | 84146 | ZNF644 | intron_08 | chr1: | chr1: | CUAAAAGUAUAAACCACACU | + |
| | | | | 90935529- | 90936709- | | |
| | | | | 90937484 | 90936728 | | |
| 1109 | 84146 | ZNF644 | intron_08 | chr1: | chr1: | AUGUUGAAGGUUUAUGUAAA | − |
| | | | | 90935529- | 90936642- | | |
| | | | | 90937484 | 90936661 | | |
| 1110 | 84146 | ZNF644 | intron_08 | chr1: | chr1: | CUCUAAGUUGAAUGUGUCAG | − |
| | | | | 90935529- | 90936605- | | |
| | | | | 90937484 | 90936624 | | |
| 1111 | 84146 | ZNF644 | intron_08 | chr1: | chr1: | AAAACUGCAUUACAACUAUU | + |
| | | | | 90935529- | 90936498- | | |
| | | | | 90937484 | 90936517 | | |
| 1112 | 84146 | ZNF644 | intron_08 | chr1: | chr1: | UGUAAUGCAGUUUUGAUUUA | − |
| | | | | 90935529- | 90936492- | | |
| | | | | 90937484 | 90936511 | | |
| 1113 | 84146 | ZNF644 | intron_08 | chr1: | chr1: | GUAUGAGCUUCUGUUUAAUC | − |
| | | | | 90935529- | 90936424- | | |
| | | | | 90937484 | 90936443 | | |
| 1114 | 84146 | ZNF644 | intron_08 | chr1: | chr1: | CUUUAUUCGAAAGAAAAAGC | + |
| | | | | 90935529- | 90936402- | | |
| | | | | 90937484 | 90936421 | | |
| 1115 | 84146 | ZNF644 | intron_08 | chr1: | chr1: | UGUGUUAGCUGUGUUGACCA | − |
| | | | | 90935529- | 90936324- | | |
| | | | | 90937484 | 90936343 | | |
| 1116 | 84146 | ZNF644 | intron_08 | chr1: | chr1: | CUCUGAAGUAUACUACCCCU | + |
| | | | | 90935529- | 90936304- | | |
| | | | | 90937484 | 90936323 | | |
| 1117 | 84146 | ZNF644 | intron_08 | chr1: | chr1: | UGAGGAUUGUCUGAGAAGUU | − |
| | | | | 90935529- | 90936219- | | |
| | | | | 90937484 | 90936238 | | |
| 1118 | 84146 | ZNF644 | intron_08 | chr1: | chr1: | GCUAACAUCUUAGUCAUCAC | + |
| | | | | 90935529- | 90936166- | | |
| | | | | 90937484 | 90936185 | | |
| 1119 | 84146 | ZNF644 | intron_08 | chr1: | chr1: | UAGGCUUUCUGAUGAAGGCU | + |
| | | | | 90935529- | 90935988- | | |
| | | | | 90937484 | 90936007 | | |
| 1120 | 84146 | ZNF644 | intron_08 | chr1: | chr1: | AUUUAGUAUUGAGACUUUUA | − |
| | | | | 90935529- | 90935968- | | |
| | | | | 90937484 | 90935987 | | |
| 1121 | 84146 | ZNF644 | intron_08 | chr1: | chr1: | UUGAGACUUUUACGGUUUUG | − |
| | | | | 90935529- | 90935960- | | |
| | | | | 90937484 | 90935979 | | |
| 1122 | 84146 | ZNF644 | intron_08 | chr1: | chr1: | UGAGACUUUUACGGUUUUGU | − |
| | | | | 90935529- | 90935959- | | |
| | | | | 90937484 | 90935978 | | |
| 1123 | 84146 | ZNF644 | intron_08 | chr1: | chr1: | AACCGUGUUGUUCAAAUCAC | − |
| | | | | 90935529- | 90935851- | | |
| | | | | 90937484 | 90935870 | | |
| 1124 | 84146 | ZNF644 | intron_08 | chr1: | chr1: | UCACAGGAUUGUAUUGUAGU | − |
| | | | | 90935529- | 90935835- | | |
| | | | | 90937484 | 90935854 | | |
| 1125 | 84146 | ZNF644 | intron_08 | chr1: | chr1: | CUUGAACCUUUGAGCUCCUC | + |
| | | | | 90935529- | 90935751- | | |
| | | | | 90937484 | 90935770 | | |
| 1126 | 84146 | ZNF644 | intron_08 | chr1: | chr1: | GUAUAUCCAGAGGAGCUCAA | − |
| | | | | 90935529- | 90935760- | | |
| | | | | 90937484 | 90935779 | | |
| 1127 | 84146 | ZNF644 | intron_08 | chr1: | chr1: | GCUCUUUGGAAAGGUAGACG | + |
| | | | | 90935529- | 90935723- | | |
| | | | | 90937484 | 90935742 | | |
| 1128 | 84146 | ZNF644 | intron_08 | chr1: | chr1: | UCAACUCAUGCUCUUUGGAA | + |
| | | | | 90935529- | 90935714- | | |
| | | | | 90937484 | 90935733 | | |
| 1129 | 84146 | ZNF644 | intron_08 | chr1: | chr1: | GGUUAACUUAAAAUAAGGAG | + |
| | | | | 90935529- | 90935649- | | |
| | | | | 90937484 | 90935668 | | |
| 1130 | 84146 | ZNF644 | intron_08 | chr1: | chr1: | AAAUAGGUUAACUUAAAAUA | + |
| | | | | 90935529- | 90935644- | | |
| | | | | 90937484 | 90935663 | | |
| 1131 | 84146 | ZNF644 | exon_09_nc | chr1: | chr1: | AAGCGCGUAAGUGGUUGGAC | + |
| | | | | 90935408- | 90935447- | | |
| | | | | 90935528 | 90935466 | | |
| 1132 | 84146 | ZNF644 | exon_09_nc | chr1: | chr1: | UGCAUAAGCGCGUAAGUGGU | + |
| | | | | 90935408- | 90935442- | | |
| | | | | 90935528 | 90935461 | | |
| 1133 | 84146 | ZNF644 | exon_09_nc | chr1: | chr1: | UUCGUGCAUAAGCGCGUAAG | + |
| | | | | 90935408- | 90935438- | | |
| | | | | 90935528 | 90935457 | | |
| 1134 | 84146 | ZNF644 | exon_09_nc | chr1: | chr1: | UAUGCACGAAAGAAGAGUGC | − |
| | | | | 90935408- | 90935428- | | |
| | | | | 90935528 | 90935447 | | |
| 1135 | 84146 | ZNF644 | exon_09_nc | chr1: | chr1: | GCUGGACUUUUGACUGGUAC | − |
| | | | | 90935408- | 90935410- | | |
| | | | | 90935528 | 90935429 | | |
| 1136 | 84146 | ZNF644 | intron_09 | chr1: | chr1: | GAAUUAACACCAGUCUGGUG | + |
| | | | | 90918392- | 90935330- | | |
| | | | | 90935407 | 90935349 | | |
| 1137 | 84146 | ZNF644 | intron_09 | chr1: | chr1: | UGAAUUAACACCAGUCUGGU | + |
| | | | | 90918392- | 90935329- | | |
| | | | | 90935407 | 90935348 | | |
| 1138 | 84146 | ZNF644 | intron_09 | chr1: | chr1: | CUGAAUUAACACCAGUCUGG | + |
| | | | | 90918392- | 90935328- | | |
| | | | | 90935407 | 90935347 | | |
| 1139 | 84146 | ZNF644 | intron_09 | chr1: | chr1: | GAUCUGAAUUAACACCAGUC | + |
| | | | | 90918392- | 90935325- | | |
| | | | | 90935407 | 90935344 | | |
| 1140 | 84146 | ZNF644 | intron_09 | chr1: | chr1: | ACAAGUUGCUGAAAUUCAAU | + |
| | | | | 90918392- | 90935259- | | |
| | | | | 90935407 | 90935278 | | |
| 1141 | 84146 | ZNF644 | intron_09 | chr1: | chr1: | UACAAGUUGCUGAAAUUCAA | + |
| | | | | 90918392- | 90935258- | | |
| | | | | 90935407 | 90935277 | | |
| 1142 | 84146 | ZNF644 | intron_09 | chr1: | chr1: | GGCUCUUAUUGACUUAAUUU | − |
| | | | | 90918392- | 90935196- | | |
| | | | | 90935407 | 90935215 | | |
| 1143 | 84146 | ZNF644 | intron_09 | chr1: | chr1: | AAAAAUUAACCCAAGAGGUG | − |
| | | | | 90918392- | 90935168- | | |
| | | | | 90935407 | 90935187 | | |
| 1144 | 84146 | ZNF644 | intron_09 | chr1: | chr1: | CUAAAAAAUAAAUGGGAUUA | + |
| | | | | 90918392- | 90935129- | | |
| | | | | 90935407 | 90935148 | | |
| 1145 | 84146 | ZNF644 | intron_09 | chr1: | chr1: | CCGACUAGAACUAUAAAGAG | + |
| | | | | 90918392- | 90935060- | | |
| | | | | 90935407 | 90935079 | | |
| 1146 | 84146 | ZNF644 | intron_09 | chr1: | chr1: | AAAAUAUUGUAGUUCUAGCU | − |
| | | | | 90918392- | 90934900- | | |
| | | | | 90935407 | 90934919 | | |
| 1147 | 84146 | ZNF644 | intron_09 | chr1: | chr1: | AAAUAUUGUAGUUCUAGCUU | − |
| | | | | 90918392- | 90934899- | | |
| | | | | 90935407 | 90934918 | | |
| 1148 | 84146 | ZNF644 | intron_09 | chr1: | chr1: | GAAGGGGUGAGCCUUAAUAA | − |
| | | | | 90918392- | 90934769- | | |
| | | | | 90935407 | 90934788 | | |
| 1149 | 84146 | ZNF644 | intron_09 | chr1: | chr1: | UAAGAGGUAUCUGCAAGGUU | + |
| | | | | 90918392- | 90934621- | | |
| | | | | 90935407 | 90934640 | | |
| 1150 | 84146 | ZNF644 | intron_09 | chr1: | chr1: | UUAAGAGGUAUCUGCAAGGU | + |
| | | | | 90918392- | 90934620- | | |
| | | | | 90935407 | 90934639 | | |
| 1151 | 84146 | ZNF644 | intron_09 | chr1: | chr1: | AGUAUUAAGAGGUAUCUGCA | + |
| | | | | 90918392- | 90934616- | | |
| | | | | 90935407 | 90934635 | | |
| 1152 | 84146 | ZNF644 | intron_09 | chr1: | chr1: | AGGGUAUAUUUUAAGAUGUG | + |
| | | | | 90918392- | 90934473- | | |
| | | | | 90935407 | 90934492 | | |
| 1153 | 84146 | ZNF644 | intron_09 | chr1: | chr1: | GAUCUAAGUUCUAGGACAUG | + |
| | | | | 90918392- | 90934323- | | |
| | | | | 90935407 | 90934342 | | |
| 1154 | 84146 | ZNF644 | intron_09 | chr1: | chr1: | ACAUAUUGGAUCUAAGUUCU | + |
| | | | | 90918392- | 90934315- | | |
| | | | | 90935407 | 90934334 | | |
| 1155 | 84146 | ZNF644 | intron_09 | chr1: | chr1: | GACACUAGUAUAAAUAUAAG | + |
| | | | | 90918392- | 90934276- | | |
| | | | | 90935407 | 90934295 | | |
| 1156 | 84146 | ZNF644 | intron_09 | chr1: | chr1: | GUCAUUUGUAUAUCAAGUUA | + |
| | | | | 90918392- | 90934229- | | |
| | | | | 90935407 | 90934248 | | |
| 1157 | 84146 | ZNF644 | intron_09 | chr1: | chr1: | AACUUGAUAUACAAAUGACC | − |
| | | | | 90918392- | 90934228- | | |
| | | | | 90935407 | 90934247 | | |
| 1158 | 84146 | ZNF644 | intron_09 | chr1: | chr1: | ACUUGAUAUACAAAUGACCA | − |
| | | | | 90918392- | 90934227- | | |
| | | | | 90935407 | 90934246 | | |
| 1159 | 84146 | ZNF644 | intron_09 | chr1: | chr1: | AAAUUGUCAACAUUCAUCCC | + |
| | | | | 90918392- | 90934207- | | |
| | | | | 90935407 | 90934226 | | |
| 1160 | 84146 | ZNF644 | intron_09 | chr1: | chr1: | UGGGGUGAGUUUUAGUCUAC | − |
| | | | | 90918392- | 90934169- | | |
| | | | | 90935407 | 90934188 | | |
| 1161 | 84146 | ZNF644 | intron_09 | chr1: | chr1: | GAGUUUUAGUCUACUGGAUU | − |
| | | | | 90918392- | 90934163- | | |
| | | | | 90935407 | 90934182 | | |
| 1162 | 84146 | ZNF644 | intron_09 | chr1: | chr1: | UAUAUACCCUCACCUUUAUC | − |
| | | | | 90918392- | 90934115- | | |
| | | | | 90935407 | 90934134 | | |
| 1163 | 84146 | ZNF644 | intron_09 | chr1: | chr1: | UGAUUGCAGACAUUAGAGGU | − |
| | | | | 90918392- | 90934083- | | |
| | | | | 90935407 | 90934102 | | |
| 1164 | 84146 | ZNF644 | intron_09 | chr1: | chr1: | GAUUGCAGACAUUAGAGGUG | − |
| | | | | 90918392- | 90934082- | | |
| | | | | 90935407 | 90934101 | | |
| 1165 | 84146 | ZNF644 | intron_09 | chr1: | chr1: | UCAAUAAAAGUUAGCAAGUA | + |
| | | | | 90918392- | 90934010- | | |
| | | | | 90935407 | 90934029 | | |
| 1166 | 84146 | ZNF644 | intron_09 | chr1: | chr1: | UCCAGUACCUACAACUUGUA | + |
| | | | | 90918392- | 90933921- | | |
| | | | | 90935407 | 90933940 | | |
| 1167 | 84146 | ZNF644 | intron_09 | chr1: | chr1: | AAAAAUAGACCGCCUGUGUA | + |
| | | | | 90918392- | 90933762- | | |
| | | | | 90935407 | 90933781 | | |
| 1168 | 84146 | ZNF644 | intron_09 | chr1: | chr1: | GAAAAUUCUAAAAUGAAGGU | − |
| | | | | 90918392- | 90933695- | | |
| | | | | 90935407 | 90933714 | | |
| 1169 | 84146 | ZNF644 | intron_09 | chr1: | chr1: | CCCAUUUUAAACCCACCUUC | + |
| | | | | 90918392- | 90933359- | | |
| | | | | 90935407 | 90933378 | | |
| 1170 | 84146 | ZNF644 | intron_09 | chr1: | chr1: | GUUUAAAAUGGGGACUAUUC | − |
| | | | | 90918392- | 90933351- | | |
| | | | | 90935407 | 90933370 | | |
| 1171 | 84146 | ZNF644 | intron_09 | chr1: | chr1: | AUUUAGGCCUAAGCACUUCU | + |
| | | | | 90918392- | 90933282- | | |
| | | | | 90935407 | 90933301 | | |
| 1172 | 84146 | ZNF644 | intron_09 | chr1: | chr1: | AGGCCUAAAUUAGACUUUAG | − |
| | | | | 90918392- | 90933272- | | |
| | | | | 90935407 | 90933291 | | |
| 1173 | 84146 | ZNF644 | intron_09 | chr1: | chr1: | AUCUGCUUGAAUAAUCAGAA | + |
| | | | | 90918392- | 90933236- | | |
| | | | | 90935407 | 90933255 | | |
| 1174 | 84146 | ZNF644 | intron_09 | chr1: | chr1: | UGGACUAAUAAAAUCUGGAC | + |
| | | | | 90918392- | 90933183- | | |
| | | | | 90935407 | 90933202 | | |
| 1175 | 84146 | ZNF644 | intron_09 | chr1: | chr1: | AAAAGUAAUGUAGGAUGACU | − |
| | | | | 90918392- | 90933027- | | |
| | | | | 90935407 | 90933046 | | |
| 1176 | 84146 | ZNF644 | intron_09 | chr1: | chr1: | AACACCCUCUUUUAUUCUUC | + |
| | | | | 90918392- | 90932988- | | |
| | | | | 90935407 | 90933007 | | |
| 1177 | 84146 | ZNF644 | intron_09 | chr1: | chr1: | ACCAAAAUAUUAGAAGUACU | − |
| | | | | 90918392- | 90932944- | | |
| | | | | 90935407 | 90932963 | | |
| 1178 | 84146 | ZNF644 | intron_09 | chr1: | chr1: | AGCGUGUGUUUGGCAGUCCU | − |
| | | | | 90918392- | 90932913- | | |
| | | | | 90935407 | 90932932 | | |
| 1179 | 84146 | ZNF644 | intron_09 | chr1: | chr1: | GGUGUGGAAAAUAAGACCCU | + |
| | | | | 90918392- | 90932855- | | |
| | | | | 90935407 | 90932874 | | |
| 1180 | 84146 | ZNF644 | intron_09 | chr1: | chr1: | UCAAAAGUGGCUACUGGGUG | + |
| | | | | 90918392- | 90932839- | | |
| | | | | 90935407 | 90932858 | | |
| 1181 | 84146 | ZNF644 | intron_09 | chr1: | chr1: | GUUUCUCAAAAGUGGCUACU | + |
| | | | | 90918392- | 90932834- | | |
| | | | | 90935407 | 90932853 | | |
| 1182 | 84146 | ZNF644 | intron_09 | chr1: | chr1: | AGUUUCUCAAAAGUGGCUAC | + |
| | | | | 90918392- | 90932833- | | |
| | | | | 90935407 | 90932852 | | |
| 1183 | 84146 | ZNF644 | intron_09 | chr1: | chr1: | UAUGGGGGAUAAUCAGUAUG | + |
| | | | | 90918392- | 90932621- | | |
| | | | | 90935407 | 90932640 | | |
| 1184 | 84146 | ZNF644 | intron_09 | chr1: | chr1: | UAACAGUGAUAUUGAUAUGG | + |
| | | | | 90918392- | 90932606- | | |
| | | | | 90935407 | 90932625 | | |
| 1185 | 84146 | ZNF644 | intron_09 | chr1: | chr1: | UUAUAACAGUGAUAUUGAUA | + |
| | | | | 90918392- | 90932603- | | |
| | | | | 90935407 | 90932622 | | |
| 1186 | 84146 | ZNF644 | intron_09 | chr1: | chr1: | UUCUAGGGCCAUAGGUUUUU | + |
| | | | | 90918392- | 90932571- | | |
| | | | | 90935407 | 90932590 | | |
| 1187 | 84146 | ZNF644 | intron_09 | chr1: | chr1: | GUGGAACCUUCUAGGGCCAU | + |
| | | | | 90918392- | 90932563- | | |
| | | | | 90935407 | 90932582 | | |
| 1188 | 84146 | ZNF644 | intron_09 | chr1: | chr1: | UGGCCCUAGAAGGUUCCACU | − |
| | | | | 90918392- | 90932562- | | |
| | | | | 90935407 | 90932581 | | |
| 1189 | 84146 | ZNF644 | intron_09 | chr1: | chr1: | AAUCACUUAUAAUAACCAAG | + |
| | | | | 90918392- | 90932544- | | |
| | | | | 90935407 | 90932563 | | |
| 1190 | 84146 | ZNF644 | intron_09 | chr1: | chr1: | CAUUAAUUCAUAUGAGUAAC | − |
| | | | | 90918392- | 90932524- | | |
| | | | | 90935407 | 90932543 | | |
| 1191 | 84146 | ZNF644 | intron_09 | chr1: | chr1: | UCCAAUUUUGUGAUAUACUA | + |
| | | | | 90918392- | 90932432- | | |
| | | | | 90935407 | 90932451 | | |
| 1192 | 84146 | ZNF644 | intron_09 | chr1: | chr1: | ACCUUAGUAUAUCACAAAAU | − |
| | | | | 90918392- | 90932436- | | |
| | | | | 90935407 | 90932455 | | |
| 1193 | 84146 | ZNF644 | intron_09 | chr1: | chr1: | CAUACUGGACAUGGUUAGGG | + |
| | | | | 90918392- | 90932377- | | |
| | | | | 90935407 | 90932396 | | |
| 1194 | 84146 | ZNF644 | intron_09 | chr1: | chr1: | ACACAUACUGGACAUGGUUA | + |
| | | | | 90918392- | 90932374- | | |
| | | | | 90935407 | 90932393 | | |
| 1195 | 84146 | ZNF644 | intron_09 | chr1: | chr1: | CUAAGCUUUGUAUUCAAGAG | + |
| | | | | 90918392- | 90932302- | | |
| | | | | 90935407 | 90932321 | | |
| 1196 | 84146 | ZNF644 | intron_09 | chr1: | chr1: | GCCUUUACCAUUUAGUCUGA | + |
| | | | | 90918392- | 90932276- | | |
| | | | | 90935407 | 90932295 | | |
| 1197 | 84146 | ZNF644 | intron_09 | chr1: | chr1: | GGCCUUUACCAUUUAGUCUG | + |
| | | | | 90918392- | 90932275- | | |
| | | | | 90935407 | 90932294 | | |
| 1198 | 84146 | ZNF644 | intron_09 | chr1: | chr1: | UUAGAUACCCUCAGACUAAA | − |
| | | | | 90918392- | 90932286- | | |
| | | | | 90935407 | 90932305 | | |
| 1199 | 84146 | ZNF644 | intron_09 | chr1: | chr1: | ACCCUCAGACUAAAUGGUAA | − |
| | | | | 90918392- | 90932280- | | |
| | | | | 90935407 | 90932299 | | |
| 1200 | 84146 | ZNF644 | intron_09 | chr1: | chr1: | UUGAAAGUUUAUUCUAUCAC | + |
| | | | | 90918392- | 90932240- | | |
| | | | | 90935407 | 90932259 | | |
| 1201 | 84146 | ZNF644 | intron_09 | chr1: | chr1: | GGGAACUGGUGACCUUUUGA | − |
| | | | | 90918392- | 90932184- | | |
| | | | | 90935407 | 90932203 | | |
| 1202 | 84146 | ZNF644 | intron_09 | chr1: | chr1: | UUACCAGUAAACAGGUAUUU | + |
| | | | | 90918392- | 90932084- | | |
| | | | | 90935407 | 90932103 | | |
| 1203 | 84146 | ZNF644 | intron_09 | chr1: | chr1: | UAUUUAAGUUACCAGUAAAC | + |
| | | | | 90918392- | 90932076- | | |
| | | | | 90935407 | 90932095 | | |
| 1204 | 84146 | ZNF644 | intron_09 | chr1: | chr1: | CAUGAAAUUCUCAAAGAUGU | + |
| | | | | 90918392- | 90932016- | | |
| | | | | 90935407 | 90932035 | | |
| 1205 | 84146 | ZNF644 | intron_09 | chr1: | chr1: | AGACUUUGCGUAUCUCCUGA | − |
| | | | | 90918392- | 90931930- | | |
| | | | | 90935407 | 90931949 | | |
| 1206 | 84146 | ZNF644 | intron_09 | chr1: | chr1: | ACAAAUUGAUGAUAGCCUUC | + |
| | | | | 90918392- | 90931912- | | |
| | | | | 90935407 | 90931931 | | |
| 1207 | 84146 | ZNF644 | intron_09 | chr1: | chr1: | GCUAUCAUCAAUUUGUCUGA | − |
| | | | | 90918392- | 90931908- | | |
| | | | | 90935407 | 90931927 | | |
| 1208 | 84146 | ZNF644 | intron_09 | chr1: | chr1: | GUCUGAUGGCAAAUUGAGAA | − |
| | | | | 90918392- | 90931894- | | |
| | | | | 90935407 | 90931913 | | |
| 1209 | 84146 | ZNF644 | intron_09 | chr1: | chr1: | UAAUAGCAAAACUAAUUGUG | − |
| | | | | 90918392- | 90931765- | | |
| | | | | 90935407 | 90931784 | | |
| 1210 | 84146 | ZNF644 | intron_09 | chr1: | chr1: | CGGUUCCUUUUACAAAUAUU | + |
| | | | | 90918392- | 90931728- | | |
| | | | | 90935407 | 90931747 | | |
| 1211 | 84146 | ZNF644 | intron_09 | chr1: | chr1: | AUAUUUGUAAAAGGAACCGA | − |
| | | | | 90918392- | 90931727- | | |
| | | | | 90935407 | 90931746 | | |
| 1212 | 84146 | ZNF644 | intron_09 | chr1: | chr1: | UAUUUGUAAAAGGAACCGAU | − |
| | | | | 90918392- | 90931726- | | |
| | | | | 90935407 | 90931745 | | |
| 1213 | 84146 | ZNF644 | intron_09 | chr1: | chr1: | UUUGUAAAAGGAACCGAUGG | − |
| | | | | 90918392- | 90931724- | | |
| | | | | 90935407 | 90931743 | | |
| 1214 | 84146 | ZNF644 | intron_09 | chr1: | chr1: | AUCUUUCCUAUUUCCCCCAU | + |
| | | | | 90918392- | 90931708- | | |
| | | | | 90935407 | 90931727 | | |
| 1215 | 84146 | ZNF644 | intron_09 | chr1: | chr1: | AAGGAACCGAUGGGGGAAAU | − |
| | | | | 90918392- | 90931717- | | |
| | | | | 90935407 | 90931736 | | |
| 1216 | 84146 | ZNF644 | intron_09 | chr1: | chr1: | UUGGAAGAGGUGGGCAGACU | − |
| | | | | 90918392- | 90931674- | | |
| | | | | 90935407 | 90931693 | | |
| 1217 | 84146 | ZNF644 | intron_09 | chr1: | chr1: | GUCCCAGUUUAUCUUCUGUU | |
| | | | | 90918392- | 90931595- | | |
| | | | | 90935407 | 90931614 | | |
| 1218 | 84146 | ZNF644 | intron_09 | chr1: | chr1: | CAGUUUAUCUUCUGUUGGGU | − |
| | | | | 90918392- | 90931591- | | |
| | | | | 90935407 | 90931610 | | |
| 1219 | 84146 | ZNF644 | intron_09 | chr1: | chr1: | GAUAAAGAUUGAUCGGGGAA | + |
| | | | | 90918392- | 90931497- | | |
| | | | | 90935407 | 90931516 | | |
| 1220 | 84146 | ZNF644 | intron_09 | chr1: | chr1: | AUUUUGAUAAAGAUUGAUCG | + |
| | | | | 90918392- | 90931492- | | |
| | | | | 90935407 | 90931511 | | |
| 1221 | 84146 | ZNF644 | intron_09 | chr1: | chr1: | UCAAAAUAGACUAGAAUCAA | − |
| | | | | 90918392- | 90931479- | | |
| | | | | 90935407 | 90931498 | | |
| 1222 | 84146 | ZNF644 | intron_09 | chr1: | chr1: | UAUGGAAUGCAGUAAUGGUA | − |
| | | | | 90918392- | 90931389- | | |
| | | | | 90935407 | 90931408 | | |
| 1223 | 84146 | ZNF644 | intron_09 | chr1: | chr1: | AUGGAAUGCAGUAAUGGUAG | − |
| | | | | 90918392- | 90931388- | | |
| | | | | 90935407 | 90931407 | | |
| 1224 | 84146 | ZNF644 | intron_09 | chr1: | chr1: | CAUCAUUUAAUUAAAACAUU | + |
| | | | | 90918392- | 90931176- | | |
| | | | | 90935407 | 90931195 | | |
| 1225 | 84146 | ZNF644 | intron_09 | chr1: | chr1: | AGUACUUAGGUGUGACAACA | + |
| | | | | 90918392- | 90931080- | | |
| | | | | 90935407 | 90931099 | | |
| 1226 | 84146 | ZNF644 | intron_09 | chr1: | chr1: | UAUCAACCUAAUCUGCAAUC | − |
| | | | | 90918392- | 90930958- | | |
| | | | | 90935407 | 90930977 | | |
| 1227 | 84146 | ZNF644 | intron_09 | chr1: | chr1: | AAGAGUGAAUUUCAACAGGU | − |
| | | | | 90918392- | 90930908- | | |
| | | | | 90935407 | 90930927 | | |
| 1228 | 84146 | ZNF644 | intron_09 | chr1: | chr1: | AGAGUGAAUUUCAACAGGUA | − |
| | | | | 90918392- | 90930907- | | |
| | | | | 90935407 | 90930926 | | |
| 1229 | 84146 | ZNF644 | intron_09 | chr1: | chr1: | UCUACCAUAGAAAAGCAUUG | + |
| | | | | 90918392- | 90930822- | | |
| | | | | 90935407 | 90930841 | | |
| 1230 | 84146 | ZNF644 | intron_09 | chr1: | chr1: | UUAAUGUAAGUUUGUACAUG | + |
| | | | | 90918392- | 90930717- | | |
| | | | | 90935407 | 90930736 | | |
| 1231 | 84146 | ZNF644 | intron_09 | chr1: | chr1: | UACAUUUAGGUGAGUACACA | + |
| | | | | 90918392- | 90930679- | | |
| | | | | 90935407 | 90930698 | | |
| 1232 | 84146 | ZNF644 | intron_09 | chr1: | chr1: | AUGAUAGUGACUUCUUAAGU | + |
| | | | | 90918392- | 90930578- | | |
| | | | | 90935407 | 90930597 | | |
| 1233 | 84146 | ZNF644 | intron_09 | chr1: | chr1: | UGCCUCUUUCUCUUAGGGGU | − |
| | | | | 90918392- | 90930536- | | |
| | | | | 90935407 | 90930555 | | |
| 1234 | 84146 | ZNF644 | intron_09 | chr1: | chr1: | UAGGGGUUGGUACUGACCAA | − |
| | | | | 90918392- | 90930523- | | |
| | | | | 90935407 | 90930542 | | |
| 1235 | 84146 | ZNF644 | intron_09 | chr1: | chr1: | AUCUAAGAUACUACUACCAU | + |
| | | | | 90918392- | 90930504- | | |
| | | | | 90935407 | 90930523 | | |
| 1236 | 84146 | ZNF644 | intron_09 | chr1: | chr1: | UAGAUGUCAUAACUCUUCUG | − |
| | | | | 90918392- | 90930489- | | |
| | | | | 90935407 | 90930508 | | |
| 1237 | 84146 | ZNF644 | intron_09 | chr1: | chr1: | GUAAAUCAAAUGCUAACCUC | − |
| | | | | 90918392- | 90930409- | | |
| | | | | 90935407 | 90930428 | | |
| 1238 | 84146 | ZNF644 | intron_09 | chr1: | chr1: | CCAAUUACUUUAAGUCCCAG | + |
| | | | | 90918392- | 90930390- | | |
| | | | | 90935407 | 90930409 | | |
| 1239 | 84146 | ZNF644 | intron_09 | chr1: | chr1: | CCUCUGGGACUUAAAGUAAU | − |
| | | | | 90918392- | 90930393- | | |
| | | | | 90935407 | 90930412 | | |
| 1240 | 84146 | ZNF644 | intron_09 | chr1: | chr1: | AACGUAUCAAGAAAGUGUUC | + |
| | | | | 90918392- | 90929983- | | |
| | | | | 90935407 | 90930002 | | |
| 1241 | 84146 | ZNF644 | intron_09 | chr1: | chr1: | AACACUUUCUUGAUACGUUU | − |
| | | | | 90918392- | 90929982- | | |
| | | | | 90935407 | 90930001 | | |
| 1242 | 84146 | ZNF644 | intron_09 | chr1: | chr1: | GUUGUAGAAUAGCAACAAUU | + |
| | | | | 90918392- | 90929948- | | |
| | | | | 90935407 | 90929967 | | |
| 1243 | 84146 | ZNF644 | intron_09 | chr1: | chr1: | UGUUGUAGAAUAGCAACAAU | + |
| | | | | 90918392- | 90929947- | | |
| | | | | 90935407 | 90929966 | | |
| 1244 | 84146 | ZNF644 | intron_09 | chr1: | chr1: | AUUAUUUGACUUUUCUGAUG | − |
| | | | | 90918392- | 90929909- | | |
| | | | | 90935407 | 90929928 | | |
| 1245 | 84146 | ZNF644 | intron_09 | chr1: | chr1: | CGAUUACACUUAAAAGUAAA | + |
| | | | | 90918392- | 90929819- | | |
| | | | | 90935407 | 90929838 | | |
| 1246 | 84146 | ZNF644 | intron_09 | chr1: | chr1: | CUUUUAAGUGUAAUCGUAGU | − |
| | | | | 90918392- | 90929815- | | |
| | | | | 90935407 | 90929834 | | |
| 1247 | 84146 | ZNF644 | intron_09 | chr1: | chr1: | AAUGAUAUAAUUGGCCACAC | − |
| | | | | 90918392- | 90929768- | | |
| | | | | 90935407 | 90929787 | | |
| 1248 | 84146 | ZNF644 | intron_09 | chr1: | chr1: | GCACAGUAUCAAGUGCUGAA | − |
| | | | | 90918392- | 90929718- | | |
| | | | | 90935407 | 90929737 | | |
| 1249 | 84146 | ZNF644 | intron_09 | chr1: | chr1: | CAUAAGAAUGACUUUUCUAG | + |
| | | | | 90918392- | 90929683- | | |
| | | | | 90935407 | 90929702 | | |
| 1250 | 84146 | ZNF644 | intron_09 | chr1: | chr1: | UUAUGUUGUGGAAAGUGCUA | − |
| | | | | 90918392- | 90929668- | | |
| | | | | 90935407 | 90929687 | | |
| 1251 | 84146 | ZNF644 | intron_09 | chr1: | chr1: | CUUGACAAUUCUUCCAGACU | − |
| | | | | 90918392- | 90929401- | | |
| | | | | 90935407 | 90929420 | | |
| 1252 | 84146 | ZNF644 | intron_09 | chr1: | chr1: | AAUUCUUCCAGACUGGGCUG | − |
| | | | | 90918392- | 90929395- | | |
| | | | | 90935407 | 90929414 | | |
| 1253 | 84146 | ZNF644 | intron_09 | chr1: | chr1: | UCUUCCAGACUGGGCUGAGG | − |
| | | | | 90918392- | 90929392- | | |
| | | | | 90935407 | 90929411 | | |
| 1254 | 84146 | ZNF644 | intron_09 | chr1: | chr1: | UAUACAACUAAAUGGCAUAU | − |
| | | | | 90918392- | 90929266- | | |
| | | | | 90935407 | 90929285 | | |
| 1255 | 84146 | ZNF644 | intron_09 | chr1: | chr1: | UUAGUCUAAGCCUCCUUUUG | − |
| | | | | 90918392- | 90929210- | | |
| | | | | 90935407 | 90929229 | | |
| 1256 | 84146 | ZNF644 | intron_09 | chr1: | chr1: | GUAAGGGCAUCAGAAAAAAA | + |
| | | | | 90918392- | 90929165- | | |
| | | | | 90935407 | 90929184 | | |
| 1257 | 84146 | ZNF644 | intron_09 | chr1: | chr1: | AUUCAAUGAGACAAACAGUA | + |
| | | | | 90918392- | 90929148- | | |
| | | | | 90935407 | 90929167 | | |
| 1258 | 84146 | ZNF644 | intron_09 | chr1: | chr1: | GGCUCAGCGAUGAACAGUCU | + |
| | | | | 90918392- | 90928981- | | |
| | | | | 90935407 | 90929000 | | |
| 1259 | 84146 | ZNF644 | intron_09 | chr1: | chr1: | UAAACAUAAUAUAACCCAGU | − |
| | | | | 90918392- | 90928935- | | |
| | | | | 90935407 | 90928954 | | |
| 1260 | 84146 | ZNF644 | intron_09 | chr1: | chr1: | CAUACUUGAUCUUUUCCCAC | + |
| | | | | 90918392- | 90928917- | | |
| | | | | 90935407 | 90928936 | | |
| 1261 | 84146 | ZNF644 | intron_09 | chr1: | chr1: | CCAUAUAAACCCUUCUGUAA | + |
| | | | | 90918392- | 90928894- | | |
| | | | | 90935407 | 90928913 | | |
| 1262 | 84146 | ZNF644 | intron_09 | chr1: | chr1: | AUCAAGUAUGCCAUUACAGA | − |
| | | | | 90918392- | 90928907- | | |
| | | | | 90935407 | 90928926 | | |
| 1263 | 84146 | ZNF644 | intron_09 | chr1: | chr1: | UCAAGUAUGCCAUUACAGAA | − |
| | | | | 90918392- | 90928906- | | |
| | | | | 90935407 | 90928925 | | |
| 1264 | 84146 | ZNF644 | intron_09 | chr1: | chr1: | CCAUUACAGAAGGGUUUAUA | − |
| | | | | 90918392- | 90928897- | | |
| | | | | 90935407 | 90928916 | | |
| 1265 | 84146 | ZNF644 | intron_09 | chr1: | chr1: | GAAGGAUGCAGUAGAGUUUU | − |
| | | | | 90918392- | 90928853- | | |
| | | | | 90935407 | 90928872 | | |
| 1266 | 84146 | ZNF644 | intron_09 | chr1: | chr1: | GAUGAAGUGUAGAAUAUUUC | − |
| | | | | 90918392- | 90928823- | | |
| | | | | 90935407 | 90928842 | | |
| 1267 | 84146 | ZNF644 | intron_09 | chr1: | chr1: | AAAUUUGGACUUCAAGUGUC | − |
| | | | | 90918392- | 90928767- | | |
| | | | | 90935407 | 90928786 | | |
| 1268 | 84146 | ZNF644 | intron_09 | chr1: | chr1: | UGGACUUCAAGUGUCAGGAG | − |
| | | | | 90918392- | 90928762- | | |
| | | | | 90935407 | 90928781 | | |
| 1269 | 84146 | ZNF644 | intron_09 | chr1: | chr1: | CAAGUGUCAGGAGUGGAACU | − |
| | | | | 90918392- | 90928755- | | |
| | | | | 90935407 | 90928774 | | |
| 1270 | 84146 | ZNF644 | intron_09 | chr1: | chr1: | AGUUCAAGUGUCAGAAGAUU | − |
| | | | | 90918392- | 90928732- | | |
| | | | | 90935407 | 90928751 | | |
| 1271 | 84146 | ZNF644 | intron_09 | chr1: | chr1: | AAGCACCAACCCCACCUUUC | + |
| | | | | 90918392- | 90928679- | | |
| | | | | 90935407 | 90928698 | | |
| 1272 | 84146 | ZNF644 | intron_09 | chr1: | chr1: | AUUGUAGUGCCAGAAAGGUG | − |
| | | | | 90918392- | 90928691- | | |
| | | | | 90935407 | 90928710 | | |
| 1273 | 84146 | ZNF644 | intron_09 | chr1: | chr1: | CAAUAACCACCAAAAUGGUU | + |
| | | | | 90918392- | 90927507- | | |
| | | | | 90935407 | 90927526 | | |
| 1274 | 84146 | ZNF644 | intron_09 | chr1: | chr1: | AAUUAUAUCCAAGCUACUCA | + |
| | | | | 90918392- | 90927459- | | |
| | | | | 90935407 | 90927478 | | |
| 1275 | 84146 | ZNF644 | intron_09 | chr1: | chr1: | AAUUAGUAGAGUAUCUAUAA | + |
| | | | | 90918392- | 90927416- | | |
| | | | | 90935407 | 90927435 | | |
| 1276 | 84146 | ZNF644 | intron_09 | chr1: | chr1: | CAAUUAGUAGAGUAUCUAUA | + |
| | | | | 90918392- | 90927415- | | |
| | | | | 90935407 | 90927434 | | |
| 1277 | 84146 | ZNF644 | intron_09 | chr1: | chr1: | AGAAAUAGCCUAAUUCUUGC | + |
| | | | | 90918392- | 90927382- | | |
| | | | | 90935407 | 90927401 | | |
| 1278 | 84146 | ZNF644 | intron_09 | chr1: | chr1: | UUAUUAUUCCAGCAAGAAUU | − |
| | | | | 90918392- | 90927393- | | |
| | | | | 90935407 | 90927412 | | |
| 1279 | 84146 | ZNF644 | intron_09 | chr1: | chr1: | CCGAUCAUUUUUUUGUAUGG | + |
| | | | | 90918392- | 90927359- | | |
| | | | | 90935407 | 90927378 | | |
| 1280 | 84146 | ZNF644 | intron_09 | chr1: | chr1: | CAGCCGAUCAUUUUUUUGUA | + |
| | | | | 90918392- | 90927356- | | |
| | | | | 90935407 | 90927375 | | |
| 1281 | 84146 | ZNF644 | intron_09 | chr1: | chr1: | CUCUGCAUACUUAGCACAGU | + |
| | | | | 90918392- | 90927297- | | |
| | | | | 90935407 | 90927316 | | |
| 1282 | 84146 | ZNF644 | intron_09 | chr1: | chr1: | UUUCACGCAUACCUCUCCAA | + |
| | | | | 90918392- | 90927203- | | |
| | | | | 90935407 | 90927222 | | |
| 1283 | 84146 | ZNF644 | intron_09 | chr1: | chr1: | AAUGCUAAUUCCAAAAGCCU | + |
| | | | | 90918392- | 90927127- | | |
| | | | | 90935407 | 90927146 | | |
| 1284 | 84146 | ZNF644 | intron_09 | chr1: | chr1: | UCCACCUUGCUCCCUCUUUC | + |
| | | | | 90918392- | 90927040- | | |
| | | | | 90935407 | 90927059 | | |
| 1285 | 84146 | ZNF644 | intron_09 | chr1: | chr1: | AGAACGCUAGUCUAAGAUGU | − |
| | | | | 90918392- | 90926933- | | |
| | | | | 90935407 | 90926952 | | |
| 1286 | 84146 | ZNF644 | intron_09 | chr1: | chr1: | UGUUGGAAUUAACUUUGUAG | − |
| | | | | 90918392- | 90926916- | | |
| | | | | 90935407 | 90926935 | | |
| 1287 | 84146 | ZNF644 | intron_09 | chr1: | chr1: | UUGGAAUUAACUUUGUAGUG | − |
| | | | | 90918392- | 90926914- | | |
| | | | | 90935407 | 90926933 | | |
| 1288 | 84146 | ZNF644 | intron_09 | chr1: | chr1: | CAGUGACAUUAAGAAUGUAA | − |
| | | | | 90918392- | 90926776- | | |
| | | | | 90935407 | 90926795 | | |
| 1289 | 84146 | ZNF644 | intron_09 | chr1: | chr1: | AGGAGGGGUACUAGAUGUAU | − |
| | | | | 90918392- | 90926756- | | |
| | | | | 90935407 | 90926775 | | |
| 1290 | 84146 | ZNF644 | intron_09 | chr1: | chr1: | UAGAUGUAUGGGUAAUAUUA | − |
| | | | | 90918392- | 90926745- | | |
| | | | | 90935407 | 90926764 | | |
| 1291 | 84146 | ZNF644 | intron_09 | chr1: | chr1: | CUCAAGAUUUUAAAUCUGAG | − |
| | | | | 90918392- | 90926679- | | |
| | | | | 90935407 | 90926698 | | |
| 1292 | 84146 | ZNF644 | intron_09 | chr1: | chr1: | UCAAGAUUUUAAAUCUGAGU | − |
| | | | | 90918392- | 90926678- | | |
| | | | | 90935407 | 90926697 | | |
| 1293 | 84146 | ZNF644 | intron_09 | chr1: | chr1: | AGAUUUUAAAUCUGAGUGGG | − |
| | | | | 90918392- | 90926675- | | |
| | | | | 90935407 | 90926694 | | |
| 1294 | 84146 | ZNF644 | intron_09 | chr1: | chr1: | UUUUAAAUCUGAGUGGGAGG | − |
| | | | | 90918392- | 90926672- | | |
| | | | | 90935407 | 90926691 | | |
| 1295 | 84146 | ZNF644 | intron_09 | chr1: | chr1: | AGCAGAUUGAUUAACUGUAC | + |
| | | | | 90918392- | 90926637- | | |
| | | | | 90935407 | 90926656 | | |
| 1296 | 84146 | ZNF644 | intron_09 | chr1: | chr1: | UGCCCUUCUACCAGACAGCA | + |
| | | | | 90918392- | 90926613- | | |
| | | | | 90935407 | 90926632 | | |
| 1297 | 84146 | ZNF644 | intron_09 | chr1: | chr1: | UACCAUGCUGUCUGGUAGAA | − |
| | | | | 90918392- | 90926618- | | |
| | | | | 90935407 | 90926637 | | |
| 1298 | 84146 | ZNF644 | intron_09 | chr1: | chr1: | ACUUUGUUAUAGUGGAUAGC | + |
| | | | | 90918392- | 90926547- | | |
| | | | | 90935407 | 90926566 | | |
| 1299 | 84146 | ZNF644 | intron_09 | chr1: | chr1: | AAGGUGAUACUUUGUUAUAG | + |
| | | | | 90918392- | 90926539- | | |
| | | | | 90935407 | 90926558 | | |
| 1300 | 84146 | ZNF644 | intron_09 | chr1: | chr1: | GGUCCAUGAAGGCUGCACAU | + |
| | | | | 90918392- | 90926179- | | |
| | | | | 90935407 | 90926198 | | |
| 1301 | 84146 | ZNF644 | intron_09 | chr1: | chr1: | UGGUUCUAGCUGGUCCAUGA | + |
| | | | | 90918392- | 90926168- | | |
| | | | | 90935407 | 90926187 | | |
| 1302 | 84146 | ZNF644 | intron_09 | chr1: | chr1: | GAGGUAAAGCAAGUCUCGUU | + |
| | | | | 90918392- | 90925966- | | |
| | | | | 90935407 | 90925985 | | |
| 1303 | 84146 | ZNF644 | intron_09 | chr1: | chr1: | AAGUUCUACAGCAGACUGAA | + |
| | | | | 90918392- | 90925873- | | |
| | | | | 90935407 | 90925892 | | |
| 1304 | 84146 | ZNF644 | intron_09 | chr1: | chr1: | ACUUACUGAUUCAACAAGUG | + |
| | | | | 90918392- | 90925817- | | |
| | | | | 90935407 | 90925836 | | |
| 1305 | 84146 | ZNF644 | intron_09 | chr1: | chr1: | GCUACAAUGGUGUAACUCUU | + |
| | | | | 90918392- | 90925774- | | |
| | | | | 90935407 | 90925793 | | |
| 1306 | 84146 | ZNF644 | intron_09 | chr1: | chr1: | UGCUGCUCAUUGGUUUCAUA | − |
| | | | | 90918392- | 90925721- | | |
| | | | | 90935407 | 90925740 | | |
| 1307 | 84146 | ZNF644 | intron_09 | chr1: | chr1: | AUUAAACCAAUAGUAAUGCU | + |
| | | | | 90918392- | 90925641- | | |
| | | | | 90935407 | 90925660 | | |
| 1308 | 84146 | ZNF644 | intron_09 | chr1: | chr1: | UAGUUUCCUAGCAUUACUAU | − |
| | | | | 90918392- | 90925650- | | |
| | | | | 90935407 | 90925669 | | |
| 1309 | 84146 | ZNF644 | intron_09 | chr1: | chr1: | GGAGGGCAAUUGUUUAAACU | + |
| | | | | 90918392- | 90925209- | | |
| | | | | 90935407 | 90925228 | | |
| 1310 | 84146 | ZNF644 | intron_09 | chr1: | chr1: | CAUGCCCCCCACUGGGGGGA | + |
| | | | | 90918392- | 90925192- | | |
| | | | | 90935407 | 90925211 | | |
| 1311 | 84146 | ZNF644 | intron_09 | chr1: | chr1: | UCAUGCCCCCCACUGGGGGG | + |
| | | | | 90918392- | 90925191- | | |
| | | | | 90935407 | 90925210 | | |
| 1312 | 84146 | ZNF644 | intron_09 | chr1: | chr1: | ACUCUCAUGCCCCCCACUGG | + |
| | | | | 90918392- | 90925187- | | |
| | | | | 90935407 | 90925206 | | |
| 1313 | 84146 | ZNF644 | intron_09 | chr1: | chr1: | UGACUCUCAUGCCCCCCACU | + |
| | | | | 90918392- | 90925185- | | |
| | | | | 90935407 | 90925204 | | |
| 1314 | 84146 | ZNF644 | intron_09 | chr1: | chr1: | UUGACUCUCAUGCCCCCCAC | + |
| | | | | 90918392- | 90925184- | | |
| | | | | 90935407 | 90925203 | | |
| 1315 | 84146 | ZNF644 | intron_09 | chr1: | chr1: | CACAGUAUGCAAGAUUUACC | + |
| | | | | 90918392- | 90925128- | | |
| | | | | 90935407 | 90925147 | | |
| 1316 | 84146 | ZNF644 | intron_09 | chr1: | chr1: | CUUGAAUGCGGUUUGUAAUC | − |
| | | | | 90918392- | 90925063- | | |
| | | | | 90935407 | 90925082 | | |
| 1317 | 84146 | ZNF644 | intron_09 | chr1: | chr1: | ACAUGCCUUGUUAUAUUAUA | + |
| | | | | 90918392- | 90925020- | | |
| | | | | 90935407 | 90925039 | | |
| 1318 | 84146 | ZNF644 | intron_09 | chr1: | chr1: | CAUUACCUAAAAUUUAGCUC | + |
| | | | | 90918392- | 90924991- | | |
| | | | | 90935407 | 90925010 | | |
| 1319 | 84146 | ZNF644 | intron_09 | chr1: | chr1: | AACACUCAGACUUUCUCUAU | − |
| | | | | 90918392- | 90924941- | | |
| | | | | 90935407 | 90924960 | | |
| 1320 | 84146 | ZNF644 | intron_09 | chr1: | chr1: | ACUCAGACUUUCUCUAUUGG | − |
| | | | | 90918392- | 90924938- | | |
| | | | | 90935407 | 90924957 | | |
| 1321 | 84146 | ZNF644 | intron_09 | chr1: | chr1: | CUAUUGGAGGUUAGGGCAAA | − |
| | | | | 90918392- | 90924925- | | |
| | | | | 90935407 | 90924944 | | |
| 1322 | 84146 | ZNF644 | intron_09 | chr1: | chr1: | AGGGCAAAAGGAUGGCUUAU | − |
| | | | | 90918392- | 90924913- | | |
| | | | | 90935407 | 90924932 | | |
| 1323 | 84146 | ZNF644 | intron_09 | chr1: | chr1: | GUAUUUAAUUGUAGCUUAGC | − |
| | | | | 90918392- | 90924773- | | |
| | | | | 90935407 | 90924792 | | |
| 1324 | 84146 | ZNF644 | intron_09 | chr1: | chr1: | UAUCUGAUCAAUACCAUGUU | − |
| | | | | 90918392- | 90924730- | | |
| | | | | 90935407 | 90924749 | | |
| 1325 | 84146 | ZNF644 | intron_09 | chr1: | chr1: | UGGUCGCAAUGGAUCUGAUU | + |
| | | | | 90918392- | 90924646- | | |
| | | | | 90935407 | 90924665 | | |
| 1326 | 84146 | ZNF644 | intron_09 | chr1: | chr1: | GCACAAAUCCAUGGUCGCAA | + |
| | | | | 90918392- | 90924635- | | |
| | | | | 90935407 | 90924654 | | |
| 1327 | 84146 | ZNF644 | intron_09 | chr1: | chr1: | AAUCAGAUCCAUUGCGACCA | − |
| | | | | 90918392- | 90924646- | | |
| | | | | 90935407 | 90924665 | | |
| 1328 | 84146 | ZNF644 | intron_09 | chr1: | chr1: | AUCUAAAUGGCACAAAUCCA | + |
| | | | | 90918392- | 90924626- | | |
| | | | | 90935407 | 90924645 | | |
| 1329 | 84146 | ZNF644 | intron_09 | chr1: | chr1: | AUAUUUACCUUAAAUUAGAU | + |
| | | | | 90918392- | 90924581- | | |
| | | | | 90935407 | 90924600 | | |
| 1330 | 84146 | ZNF644 | intron_09 | chr1: | chr1: | UGUGAGUCCUAUCUAAUUUA | − |
| | | | | 90918392- | 90924591- | | |
| | | | | 90935407 | 90924610 | | |
| 1331 | 84146 | ZNF644 | intron_09 | chr1: | chr1: | CAGAAAUACGGAGAUUUUAG | − |
| | | | | 90918392- | 90924452- | | |
| | | | | 90935407 | 90924471 | | |
| 1332 | 84146 | ZNF644 | intron_09 | chr1: | chr1: | AGAAAUACGGAGAUUUUAGA | − |
| | | | | 90918392- | 90924451- | | |
| | | | | 90935407 | 90924470 | | |
| 1333 | 84146 | ZNF644 | intron_09 | chr1: | chr1: | AUACGGAGAUUUUAGAGGGC | − |
| | | | | 90918392- | 90924447- | | |
| | | | | 90935407 | 90924466 | | |
| 1334 | 84146 | ZNF644 | intron_09 | chr1: | chr1: | ACAAAAGUGGUUGAUGUUGA | − |
| | | | | 90918392- | 90924328- | | |
| | | | | 90935407 | 90924347 | | |
| 1335 | 84146 | ZNF644 | intron_09 | chr1: | chr1: | GCCGUUUUAUUAAGCAAAAU | + |
| | | | | 90918392- | 90924296- | | |
| | | | | 90935407 | 90924315 | | |
| 1336 | 84146 | ZNF644 | intron_09 | chr1: | chr1: | GCCUAUUUUGCUUAAUAAAA | − |
| | | | | 90918392- | 90924300- | | |
| | | | | 90935407 | 90924319 | | |
| 1337 | 84146 | ZNF644 | intron_09 | chr1: | chr1: | GCAAACGUAUUACUUCUUUG | + |
| | | | | 90918392- | 90924217- | | |
| | | | | 90935407 | 90924236 | | |
| 1338 | 84146 | ZNF644 | intron_09 | chr1: | chr1: | UACUGGUCAGCUGAGUUGAU | + |
| | | | | 90918392- | 90924191- | | |
| | | | | 90935407 | 90924210 | | |
| 1339 | 84146 | ZNF644 | intron_09 | chr1: | chr1: | CACAACUACAAGUGGCUUAC | + |
| | | | | 90918392- | 90924174- | | |
| | | | | 90935407 | 90924193 | | |
| 1340 | 84146 | ZNF644 | intron_09 | chr1: | chr1: | AACAAUAUCACAACUACAAG | + |
| | | | | 90918392- | 90924166- | | |
| | | | | 90935407 | 90924185 | | |
| 1341 | 84146 | ZNF644 | intron_09 | chr1: | chr1: | UAUAAACAGGAAUUAGAGGC | + |
| | | | | 90918392- | 90924108- | | |
| | | | | 90935407 | 90924127 | | |
| 1342 | 84146 | ZNF644 | intron_09 | chr1: | chr1: | UAGAGGGGAUGAUUACUAAA | + |
| | | | | 90918392- | 90924011- | | |
| | | | | 90935407 | 90924030 | | |
| 1343 | 84146 | ZNF644 | intron_09 | chr1: | chr1: | UCCACUAUUUCAAAGUAGAG | + |
| | | | | 90918392- | 90923996- | | |
| | | | | 90935407 | 90924015 | | |
| 1344 | 84146 | ZNF644 | intron_09 | chr1: | chr1: | CUCCACUAUUUCAAAGUAGA | + |
| | | | | 90918392- | 90923995- | | |
| | | | | 90935407 | 90924014 | | |
| 1345 | 84146 | ZNF644 | intron_09 | chr1: | chr1: | AAUUAUUAGUCCACAAGGAC | + |
| | | | | 90918392- | 90923824- | | |
| | | | | 90935407 | 90923843 | | |
| 1346 | 84146 | ZNF644 | intron_09 | chr1: | chr1: | UUUACAAUUAUUAGUCCACA | + |
| | | | | 90918392- | 90923819- | | |
| | | | | 90935407 | 90923838 | | |
| 1347 | 84146 | ZNF644 | intron_09 | chr1: | chr1: | UUUUUUUCAGUCAUUGGGAG | − |
| | | | | 90918392- | 90923661- | | |
| | | | | 90935407 | 90923680 | | |
| 1348 | 84146 | ZNF644 | intron_09 | chr1: | chr1: | GAAGUAUAAAGAUACACAUU | + |
| | | | | 90918392- | 90923528- | | |
| | | | | 90935407 | 90923547 | | |
| 1349 | 84146 | ZNF644 | intron_09 | chr1: | chr1: | AUACUUCAUGCCUGACUGUA | − |
| | | | | 90918392- | 90923515- | | |
| | | | | 90935407 | 90923534 | | |
| 1350 | 84146 | ZNF644 | intron_09 | chr1: | chr1: | AACUGAUGUUGAUAGGAGUA | − |
| | | | | 90918392- | 90923423- | | |
| | | | | 90935407 | 90923442 | | |
| 1351 | 84146 | ZNF644 | intron_09 | chr1: | chr1: | AUUUUAAUACUGUGUAGAGU | − |
| | | | | 90918392- | 90923388- | | |
| | | | | 90935407 | 90923407 | | |
| 1352 | 84146 | ZNF644 | intron_09 | chr1: | chr1: | UCUCUCGGCCCAUUCAAAAA | + |
| | | | | 90918392- | 90923331- | | |
| | | | | 90935407 | 90923350 | | |
| 1353 | 84146 | ZNF644 | intron_09 | chr1: | chr1: | UUGAAUGGGCCGAGAGAUUG | − |
| | | | | 90918392- | 90923328- | | |
| | | | | 90935407 | 90923347 | | |
| 1354 | 84146 | ZNF644 | intron_09 | chr1: | chr1: | UGAAUGGGCCGAGAGAUUGA | − |
| | | | | 90918392- | 90923327- | | |
| | | | | 90935407 | 90923346 | | |
| 1355 | 84146 | ZNF644 | intron_09 | chr1: | chr1: | GAAUGGGCCGAGAGAUUGAG | − |
| | | | | 90918392- | 90923326- | | |
| | | | | 90935407 | 90923345 | | |
| 1356 | 84146 | ZNF644 | intron_09 | chr1: | chr1: | AUUUAUGAUAGUAGAUACUC | − |
| | | | | 90918392- | 90923280- | | |
| | | | | 90935407 | 90923299 | | |
| 1357 | 84146 | ZNF644 | intron_09 | chr1: | chr1: | GACUUUGCAAAUAGCAGCCC | + |
| | | | | 90918392- | 90923252- | | |
| | | | | 90935407 | 90923271 | | |
| 1358 | 84146 | ZNF644 | intron_09 | chr1: | chr1: | CCAACUUACACAGUCACAUA | + |
| | | | | 90918392- | 90923138- | | |
| | | | | 90935407 | 90923157 | | |
| 1359 | 84146 | ZNF644 | intron_09 | chr1: | chr1: | UCCAACUUACACAGUCACAU | + |
| | | | | 90918392- | 90923137- | | |
| | | | | 90935407 | 90923156 | | |
| 1360 | 84146 | ZNF644 | intron_09 | chr1: | chr1: | GCCUAGGAGACCAGCUUCAU | + |
| | | | | 90918392- | 90923112- | | |
| | | | | 90935407 | 90923131 | | |
| 1361 | 84146 | ZNF644 | intron_09 | chr1: | chr1: | UGCCUAGGAGACCAGCUUCA | + |
| | | | | 90918392- | 90923111- | | |
| | | | | 90935407 | 90923130 | | |
| 1362 | 84146 | ZNF644 | intron_09 | chr1: | chr1: | UCUUUCUGUACAAAGUGCCU | + |
| | | | | 90918392- | 90923096- | | |
| | | | | 90935407 | 90923115 | | |
| 1363 | 84146 | ZNF644 | intron_09 | chr1: | chr1: | AACUUUGGCUUCUCAGCAAU | − |
| | | | | 90918392- | 90923054- | | |
| | | | | 90935407 | 90923073 | | |
| 1364 | 84146 | ZNF644 | intron_09 | chr1: | chr1: | GCUUGUGUAAAACAGGAAUG | + |
| | | | | 90918392- | 90923019- | | |
| | | | | 90935407 | 90923038 | | |
| 1365 | 84146 | ZNF644 | intron_09 | chr1: | chr1: | AGCUUGUGUAAAACAGGAAU | + |
| | | | | 90918392- | 90923018- | | |
| | | | | 90935407 | 90923037 | | |
| 1366 | 84146 | ZNF644 | intron_09 | chr1: | chr1: | UAGCUUGUGUAAAACAGGAA | + |
| | | | | 90918392- | 90923017- | | |
| | | | | 90935407 | 90923036 | | |
| 1367 | 84146 | ZNF644 | intron_09 | chr1: | chr1: | CAUGCUAGCUUGUGUAAAAC | + |
| | | | | 90918392- | 90923012- | | |
| | | | | 90935407 | 90923031 | | |
| 1368 | 84146 | ZNF644 | intron_09 | chr1: | chr1: | GCAGUCUUUUUAUAUGCUUG | − |
| | | | | 90918392- | 90922913- | | |
| | | | | 90935407 | 90922932 | | |
| 1369 | 84146 | ZNF644 | intron_09 | chr1: | chr1: | UUGGGGCUUAUGAACCUUGA | − |
| | | | | 90918392- | 90922896- | | |
| | | | | 90935407 | 90922915 | | |
| 1370 | 84146 | ZNF644 | intron_09 | chr1: | chr1: | AGAGGAUGCACACAUGCCAA | + |
| | | | | 90918392- | 90922531- | | |
| | | | | 90935407 | 90922550 | | |
| 1371 | 84146 | ZNF644 | intron_09 | chr1: | chr1: | GCUGGAGGUCGUGCUGGCAG | + |
| | | | | 90918392- | 90922498- | | |
| | | | | 90935407 | 90922517 | | |
| 1372 | 84146 | ZNF644 | intron_09 | chr1: | chr1: | UAUUAAAAAGUUGGUGCUGG | + |
| | | | | 90918392- | 90922483- | | |
| | | | | 90935407 | 90922502 | | |
| 1373 | 84146 | ZNF644 | intron_09 | chr1: | chr1: | GGAAUAAACAUGACACGAAC | + |
| | | | | 90918392- | 90922441- | | |
| | | | | 90935407 | 90922460 | | |
| 1374 | 84146 | ZNF644 | intron_09 | chr1: | chr1: | UUGGCCUGUGUUAAGUUUGG | + |
| | | | | 90918392- | 90922382- | | |
| | | | | 90935407 | 90922401 | | |
| 1375 | 84146 | ZNF644 | intron_09 | chr1: | chr1: | UGGUUGGCCUGUGUUAAGUU | + |
| | | | | 90918392- | 90922379- | | |
| | | | | 90935407 | 90922398 | | |
| 1376 | 84146 | ZNF644 | intron_09 | chr1: | chr1: | AAAUUUUCUUCCCUCUCGUA | − |
| | | | | 90918392- | 90922349- | | |
| | | | | 90935407 | 90922368 | | |
| 1377 | 84146 | ZNF644 | intron_09 | chr1: | chr1: | GCACAGUAUACCCCUACGAG | + |
| | | | | 90918392- | 90922335- | | |
| | | | | 90935407 | 90922354 | | |
| 1378 | 84146 | ZNF644 | intron_09 | chr1: | chr1: | AAUUUUCUUCCCUCUCGUAG | − |
| | | | | 90918392- | 90922348- | | |
| | | | | 90935407 | 90922367 | | |
| 1379 | 84146 | ZNF644 | intron_09 | chr1: | chr1: | GUAUCUAUACCCAAAGUAAG | + |
| | | | | 90918392- | 90922313- | | |
| | | | | 90935407 | 90922332 | | |
| 1380 | 84146 | ZNF644 | intron_09 | chr1: | chr1: | GUAUACUGUGCCUCUUACUU | − |
| | | | | 90918392- | 90922326- | | |
| | | | | 90935407 | 90922345 | | |
| 1381 | 84146 | ZNF644 | intron_09 | chr1: | chr1: | CUAGGACAAAGUGAAACAAC | + |
| | | | | 90918392- | 90922289- | | |
| | | | | 90935407 | 90922308 | | |
| 1382 | 84146 | ZNF644 | intron_09 | chr1: | chr1: | AGACUUUUAAGUUAUUGGGC | − |
| | | | | 90918392- | 90922230- | | |
| | | | | 90935407 | 90922249 | | |
| 1383 | 84146 | ZNF644 | intron_09 | chr1: | chr1: | UACAAAUAAAAAUACUUCGG | − |
| | | | | 90918392- | 90922197- | | |
| | | | | 90935407 | 90922216 | | |
| 1384 | 84146 | ZNF644 | intron_09 | chr1: | chr1: | GGCGAAUGUUAAUUUAGUUA | |
| | | | | 90918392- | 90922176- | | |
| | | | | 90935407 | 90922195 | | |
| 1385 | 84146 | ZNF644 | intron_09 | chr1: | chr1: | UUGGGUCAUUUAUAUCGUGA | − |
| | | | | 90918392- | 90922105- | | |
| | | | | 90935407 | 90922124 | | |
| 1386 | 84146 | ZNF644 | intron_09 | chr1: | chr1: | UUAUUUGCUUUGCACCAAUC | − |
| | | | | 90918392- | 90921912- | | |
| | | | | 90935407 | 90921931 | | |
| 1387 | 84146 | ZNF644 | intron_09 | chr1: | chr1: | GCCAAAGCACUAAACCAGAU | + |
| | | | | 90918392- | 90921895- | | |
| | | | | 90935407 | 90921914 | | |
| 1388 | 84146 | ZNF644 | intron_09 | chr1: | chr1: | ACCAAUCUGGUUUAGUGCUU | − |
| | | | | 90918392- | 90921899- | | |
| | | | | 90935407 | 90921918 | | |
| 1389 | 84146 | ZNF644 | intron_09 | chr1: | chr1: | UUGGCAUGUUGUUGUCGUCA | − |
| | | | | 90918392- | 90921880- | | |
| | | | | 90935407 | 90921899 | | |
| 1390 | 84146 | ZNF644 | intron_09 | chr1: | chr1: | GUUGUUGUCGUCAUGGUCAU | − |
| | | | | 90918392- | 90921873- | | |
| | | | | 90935407 | 90921892 | | |
| 1391 | 84146 | ZNF644 | intron_09 | chr1: | chr1: | GUAUUCAUAGCAUAGCAGUG | − |
| | | | | 90918392- | 90921845- | | |
| | | | | 90935407 | 90921864 | | |
| 1392 | 84146 | ZNF644 | intron_09 | chr1: | chr1: | GGAGAAUAAAUUGAGCUGUA | + |
| | | | | 90918392- | 90921709- | | |
| | | | | 90935407 | 90921728 | | |
| 1393 | 84146 | ZNF644 | intron_09 | chr1: | chr1: | UCCUAAGAUUACUAUUCGGU | + |
| | | | | 90918392- | 90921583- | | |
| | | | | 90935407 | 90921602 | | |
| 1394 | 84146 | ZNF644 | intron_09 | chr1: | chr1: | UUCCUAAGAUUACUAUUCGG | + |
| | | | | 90918392- | 90921582- | | |
| | | | | 90935407 | 90921601 | | |
| 1395 | 84146 | ZNF644 | intron_09 | chr1: | chr1: | ACCUUCCUAAGAUUACUAUU | + |
| | | | | 90918392- | 90921579- | | |
| | | | | 90935407 | 90921598 | | |
| 1396 | 84146 | ZNF644 | intron_09 | chr1: | chr1: | GCCCACCGAAUAGUAAUCUU | − |
| | | | | 90918392- | 90921587- | | |
| | | | | 90935407 | 90921606 | | |
| 1397 | 84146 | ZNF644 | intron_09 | chr1: | chr1: | ACCGAAUAGUAAUCUUAGGA | − |
| | | | | 90918392- | 90921583- | | |
| | | | | 90935407 | 90921602 | | |
| 1398 | 84146 | ZNF644 | intron_09 | chr1: | chr1: | UUAGUGACCAAAAAACUCCA | + |
| | | | | 90918392- | 90921394- | | |
| | | | | 90935407 | 90921413 | | |
| 1399 | 84146 | ZNF644 | intron_09 | chr1: | chr1: | UUGUGUAACAGUUAUCUAUU | − |
| | | | | 90918392- | 90921339- | | |
| | | | | 90935407 | 90921358 | | |
| 1400 | 84146 | ZNF644 | intron_09 | chr1: | chr1: | GUUUUGUUCUGGACUGUACC | + |
| | | | | 90918392- | 90921196- | | |
| | | | | 90935407 | 90921215 | | |
| 1401 | 84146 | ZNF644 | intron_09 | chr1: | chr1: | UAUAGGAUCCUCUACAUAAA | + |
| | | | | 90918392- | 90921066- | | |
| | | | | 90935407 | 90921085 | | |
| 1402 | 84146 | ZNF644 | intron_09 | chr1: | chr1: | UAUUAAAACUGCACAAAAAG | + |
| | | | | 90918392- | 90921036- | | |
| | | | | 90935407 | 90921055 | | |
| 1403 | 84146 | ZNF644 | intron_09 | chr1: | chr1: | GUGAUACUGCCCAUACCUUU | + |
| | | | | 90918392- | 90920984- | | |
| | | | | 90935407 | 90921003 | | |
| 1404 | 84146 | ZNF644 | intron_09 | chr1: | chr1: | AACAGAUUUUCCCAAAGGUA | − |
| | | | | 90918392- | 90920997- | | |
| | | | | 90935407 | 90921016 | | |
| 1405 | 84146 | ZNF644 | intron_09 | chr1: | chr1: | UGUGAUACUGCCCAUACCUU | + |
| | | | | 90918392- | 90920983- | | |
| | | | | 90935407 | 90921002 | | |
| 1406 | 84146 | ZNF644 | intron_09 | chr1: | chr1: | AUGGGCAGUAUCACAGGAGU | − |
| | | | | 90918392- | 90920978- | | |
| | | | | 90935407 | 90920997 | | |
| 1407 | 84146 | ZNF644 | intron_09 | chr1: | chr1: | UAUUAAAAAUCUGGGAACUU | + |
| | | | | 90918392- | 90920817- | | |
| | | | | 90935407 | 90920836 | | |
| 1408 | 84146 | ZNF644 | intron_09 | chr1: | chr1: | AACCCAGGGCUCCAAAGUUA | + |
| | | | | 90918392- | 90920741- | | |
| | | | | 90935407 | 90920760 | | |
| 1409 | 84146 | ZNF644 | intron_09 | chr1: | chr1: | AGAUUCUGUAACCUUAACUU | − |
| | | | | 90918392- | 90920755- | | |
| | | | | 90935407 | 90920774 | | |
| 1410 | 84146 | ZNF644 | intron_09 | chr1: | chr1: | UAACCUUAACUUUGGAGCCC | − |
| | | | | 90918392- | 90920747- | | |
| | | | | 90935407 | 90920766 | | |
| 1411 | 84146 | ZNF644 | intron_09 | chr1: | chr1: | AUGAUUGAUCAGUCAUAAGC | − |
| | | | | 90918392- | 90920715- | | |
| | | | | 90935407 | 90920734 | | |
| 1412 | 84146 | ZNF644 | intron_09 | chr1: | chr1: | ACUCAUUGCUACAAAAAUGA | − |
| | | | | 90918392- | 90920652- | | |
| | | | | 90935407 | 90920671 | | |
| 1413 | 84146 | ZNF644 | intron_09 | chr1: | chr1: | CUGUGGAAACUCUUAAGUUU | + |
| | | | | 90918392- | 90920585- | | |
| | | | | 90935407 | 90920604 | | |
| 1414 | 84146 | ZNF644 | intron_09 | chr1: | chr1: | UUUUGAUGCUUUAUGGACUG | + |
| | | | | 90918392- | 90920568- | | |
| | | | | 90935407 | 90920587 | | |
| 1415 | 84146 | ZNF644 | intron_09 | chr1: | chr1: | UUAUGGCUUUUGAUGCUUUA | + |
| | | | | 90918392- | 90920561- | | |
| | | | | 90935407 | 90920580 | | |
| 1416 | 84146 | ZNF644 | intron_09 | chr1: | chr1: | UCACAGAAUGUUAAUGUUAG | + |
| | | | | 90918392- | 90920354- | | |
| | | | | 90935407 | 90920373 | | |
| 1417 | 84146 | ZNF644 | intron_09 | chr1: | chr1: | UCUUACUUUGAUGAGCUUGC | + |
| | | | | 90918392- | 90920231- | | |
| | | | | 90935407 | 90920250 | | |
| 1418 | 84146 | ZNF644 | intron_09 | chr1: | chr1: | UUGGUGUAUCAUAAGAUUAC | + |
| | | | | 90918392- | 90920203- | | |
| | | | | 90935407 | 90920222 | | |
| 1419 | 84146 | ZNF644 | intron_09 | chr1: | chr1: | UGUGUUAGGCAGAAUAGAGC | + |
| | | | | 90918392- | 90920177- | | |
| | | | | 90935407 | 90920196 | | |
| 1420 | 84146 | ZNF644 | intron_09 | chr1: | chr1: | CAAGGAUCCAUCUUUACAGA | − |
| | | | | 90918392- | 90920155- | | |
| | | | | 90935407 | 90920174 | | |
| 1421 | 84146 | ZNF644 | intron_09 | chr1: | chr1: | UACACUAGAAAUAGAGUUCA | + |
| | | | | 90918392- | 90920089- | | |
| | | | | 90935407 | 90920108 | | |
| 1422 | 84146 | ZNF644 | intron_09 | chr1: | chr1: | AUACACUAGAAAUAGAGUUC | + |
| | | | | 90918392- | 90920088- | | |
| | | | | 90935407 | 90920107 | | |
| 1423 | 84146 | ZNF644 | intron_09 | chr1: | chr1: | GAUUGCUCACCAAAGCGAUU | − |
| | | | | 90918392- | 90920041- | | |
| | | | | 90935407 | 90920060 | | |
| 1424 | 84146 | ZNF644 | intron_09 | chr1: | chr1: | CACCAAAGCGAUUAGGAAAA | − |
| | | | | 90918392- | 90920034- | | |
| | | | | 90935407 | 90920053 | | |
| 1425 | 84146 | ZNF644 | intron_09 | chr1: | chr1: | CCUUAUGCUUUGUACCCAGU | + |
| | | | | 90918392- | 90920003- | | |
| | | | | 90935407 | 90920022 | | |
| 1426 | 84146 | ZNF644 | intron_09 | chr1: | chr1: | CCUACUGGGUACAAAGCAUA | − |
| | | | | 90918392- | 90920006- | | |
| | | | | 90935407 | 90920025 | | |
| 1427 | 84146 | ZNF644 | intron_09 | chr1: | chr1: | CCUAGUUUCUGUACAUAAGC | − |
| | | | | 90918392- | 90919954- | | |
| | | | | 90935407 | 90919973 | | |
| 1428 | 84146 | ZNF644 | intron_09 | chr1: | chr1: | GUUUCUGUACAUAAGCUGGC | − |
| | | | | 90918392- | 90919950- | | |
| | | | | 90935407 | 90919969 | | |
| 1429 | 84146 | ZNF644 | intron_09 | chr1: | chr1: | CUGGUCAGUGAUUUAUAUCU | − |
| | | | | 90918392- | 90919931- | | |
| | | | | 90935407 | 90919950 | | |
| 1430 | 84146 | ZNF644 | intron_09 | chr1: | chr1: | AAUGAUGCACAAUGUUGUAC | − |
| | | | | 90918392- | 90919869- | | |
| | | | | 90935407 | 90919888 | | |
| 1431 | 84146 | ZNF644 | intron_09 | chr1: | chr1: | GUUUUACUCCUAUUUAAUAC | + |
| | | | | 90918392- | 90919836- | | |
| | | | | 90935407 | 90919855 | | |
| 1432 | 84146 | ZNF644 | intron_09 | chr1: | chr1: | GAACUACUAGAAGUCAAGUU | + |
| | | | | 90918392- | 90919624- | | |
| | | | | 90935407 | 90919643 | | |
| 1433 | 84146 | ZNF644 | intron_09 | chr1: | chr1: | CUUUUGAAUAGGCAGGUGAC | − |
| | | | | 90918392- | 90919545- | | |
| | | | | 90935407 | 90919564 | | |
| 1434 | 84146 | ZNF644 | intron_09 | chr1: | chr1: | CAGUGGCUUCAAUGAGAUUU | − |
| | | | | 90918392- | 90919512- | | |
| | | | | 90935407 | 90919531 | | |
| 1435 | 84146 | ZNF644 | intron_09 | chr1: | chr1: | GACUAAGGAACUAUGUUUAC | − |
| | | | | 90918392- | 90919437- | | |
| | | | | 90935407 | 90919456 | | |
| 1436 | 84146 | ZNF644 | intron_09 | chr1: | chr1: | UGUAGGGAUCCAAGGGGGGA | + |
| | | | | 90918392- | 90919238- | | |
| | | | | 90935407 | 90919257 | | |
| 1437 | 84146 | ZNF644 | intron_09 | chr1: | chr1: | CAUUUGUAUCCCUCCCCCCU | − |
| | | | | 90918392- | 90919250- | | |
| | | | | 90935407 | 90919269 | | |
| 1438 | 84146 | ZNF644 | intron_09 | chr1: | chr1: | UUAUCUGUAGGGAUCCAAGG | + |
| | | | | 90918392- | 90919233- | | |
| | | | | 90935407 | 90919252 | | |
| 1439 | 84146 | ZNF644 | intron_09 | chr1: | chr1: | AAUUUAUCUGUAGGGAUCCA | + |
| | | | | 90918392- | 90919230- | | |
| | | | | 90935407 | 90919249 | | |
| 1440 | 84146 | ZNF644 | intron_09 | chr1: | chr1: | AAGGUAAGAAUUUAUCUGUA | + |
| | | | | 90918392- | 90919222- | | |
| | | | | 90935407 | 90919241 | | |
| 1441 | 84146 | ZNF644 | intron_09 | chr1: | chr1: | GGUCAAUUAGCAGGAAGAGA | + |
| | | | | 90918392- | 90919203- | | |
| | | | | 90935407 | 90919222 | | |
| 1442 | 84146 | ZNF644 | intron_09 | chr1: | chr1: | CUAAUUGACCAGCAGCACCC | |
| | | | | 90918392- | 90919193- | | |
| | | | | 90935407 | 90919212 | | |
| 1443 | 84146 | ZNF644 | intron_09 | chr1: | chr1: | AAAGGGAUGAUAGCAAUCCA | + |
| | | | | 90918392- | 90919173- | | |
| | | | | 90935407 | 90919192 | | |
| 1444 | 84146 | ZNF644 | intron_09 | chr1: | chr1: | GAUAGCACAUAACAUUUUAU | + |
| | | | | 90918392- | 90919118- | | |
| | | | | 90935407 | 90919137 | | |
| 1445 | 84146 | ZNF644 | intron_09 | chr1: | chr1: | GGUUGUUUUAGAAUAAAUAA | − |
| | | | | 90918392- | 90919055- | | |
| | | | | 90935407 | 90919074 | | |
| 1446 | 84146 | ZNF644 | intron_09 | chr1: | chr1: | AUCUCACGUAAGUUUACAAU | + |
| | | | | 90918392- | 90918870- | | |
| | | | | 90935407 | 90918889 | | |
| 1447 | 84146 | ZNF644 | intron_09 | chr1: | chr1: | UAAACUUACGUGAGAUAAUA | − |
| | | | | 90918392- | 90918866- | | |
| | | | | 90935407 | 90918885 | | |
| 1448 | 84146 | ZNF644 | intron_09 | chr1: | chr1: | UAGCUCUUAAAAUUGUAUGC | + |
| | | | | 90918392- | 90918719- | | |
| | | | | 90935407 | 90918738 | | |
| 1449 | 84146 | ZNF644 | intron_09 | chr1: | chr1: | GAAAACUUCGUAAGGGAAUA | + |
| | | | | 90918392- | 90918629- | | |
| | | | | 90935407 | 90918648 | | |
| 1450 | 84146 | ZNF644 | intron_09 | chr1: | chr1: | AUACUUGGAAAACUUCGUAA | + |
| | | | | 90918392- | 90918622- | | |
| | | | | 90935407 | 90918641 | | |
| 1451 | 84146 | ZNF644 | intron_09 | chr1: | chr1: | UAUACUUGGAAAACUUCGUA | + |
| | | | | 90918392- | 90918621- | | |
| | | | | 90935407 | 90918640 | | |
| 1452 | 84146 | ZNF644 | intron_09 | chr1: | chr1: | AGUAUAGAAGACCUACUUAA | − |
| | | | | 90918392- | 90918607- | | |
| | | | | 90935407 | 90918626 | | |
| 1453 | 84146 | ZNF644 | intron_09 | chr1: | chr1: | UUCUCAGUUCUAGUUUGGUG | − |
| | | | | 90918392- | 90918417- | | |
| | | | | 90935407 | 90918436 | | |
| 1454 | 84146 | ZNF644 | exon_10_nc.1 | chr1: | chr1: | AUUAUUUGAAAAGGCAGUAU | − |
| | | | | 90918155- | 90918388- | | |
| | | | | 90918391 | 90918407 | | |
| 1455 | 84146 | ZNF644 | exon_10_nc.1 | chr1: | chr1: | AUAUUAUCUGUACUGUACAG | + |
| | | | | 90918155- | 90918327- | | |
| | | | | 90918391 | 90918346 | | |
| 1456 | 84146 | ZNF644 | exon_10_nc.1 | chr1: | chr1: | CUUACAAUCUAAGGCUAAAA | + |
| | | | | 90918155- | 90918141- | | |
| | | | | 90918391 | 90918160 | | |
| 1457 | 84146 | ZNF644 | exon_10_c.1/ | chr1: | chr1: | UAAACCUCGUCAGCACCAUG | + |
| | | | nc.2 | 90918052- | 90918067- | | |
| | | | | 90918154 | 90918086 | | |
| 1458 | 84146 | ZNF644 | intron_10 | chr1: | chr1: | GUCUUGAGUUCUCGCUUAUU | − |
| | | | | 90916991- | 90917416- | | |
| | | | | 90918051 | 90917435 | | |
| 1459 | 84146 | ZNF644 | intron_10 | chr1: | chr1: | GAAGAAGGCGUGACUAUGAG | + |
| | | | | 90916991- | 90917376- | | |
| | | | | 90918051 | 90917395 | | |
| 1460 | 84146 | ZNF644 | intron_10 | chr1: | chr1: | CCAGUUUAGCAUUCCUUAAA | + |
| | | | | 90916991- | 90917305- | | |
| | | | | 90918051 | 90917324 | | |
| 1461 | 84146 | ZNF644 | intron_10 | chr1: | chr1: | CCAUUUAAGGAAUGCUAAAC | − |
| | | | | 90916991- | 90917308- | | |
| | | | | 90918051 | 90917327 | | |
| 1462 | 84146 | ZNF644 | intron_10 | chr1: | chr1: | AAGGAAUGCUAAACUGGGGA | − |
| | | | | 90916991- | 90917302- | | |
| | | | | 90918051 | 90917321 | | |
| 1463 | 84146 | ZNF644 | intron_10 | chr1: | chr1: | CCAGUAAUCUUUGUAUAGGU | + |
| | | | | 90916991- | 90917185- | | |
| | | | | 90918051 | 90917204 | | |
| 1464 | 84146 | ZNF644 | intron_10 | chr1: | chr1: | AACUCCAGUAAUCUUUGUAU | + |
| | | | | 90916991- | 90917181- | | |
| | | | | 90918051 | 90917200 | | |
| 1465 | 84146 | ZNF644 | intron_10 | chr1: | chr1: | CCCACCUAUACAAAGAUUAC | − |
| | | | | 90916991- | 90917188- | | |
| | | | | 90918051 | 90917207 | | |
| 1466 | 84146 | ZNF644 | exon_11_c.1/ | chr1: | chr1: | ACAAGGGUCCUCGAAAGACU | + |
| | | | nc.1 | 90916801- | 90916959- | | |
| | | | | 90916990 | 90916978 | | |
| 1467 | 84146 | ZNF644 | exon_11_c.1/ | chr1: | chr1: | UUUUGUGGCCUAGUCUUUCG | − |
| | | | nc.1 | 90916801- | 90916970- | | |
| | | | | 90916990 | 90916989 | | |
| 1468 | 84146 | ZNF644 | exon_11_c.1/ | chr1: | chr1: | CCAGUCUUCCUGAACAGACA | + |
| | | | nc.1 | 90916801- | 90916942- | | |
| | | | | 90916990 | 90916961 | | |
| 1469 | 84146 | ZNF644 | exon_11_c.1/ | chr1: | chr1: | UCGAGGACCCUUGUCUGUUC | − |
| | | | nc.1 | 90916801- | 90916953- | | |
| | | | | 90916990 | 90916972 | | |
| 1470 | 84146 | ZNF644 | exon_11_c.1/ | chr1: | chr1: | UUGUAAACGCUAAUCUUCCA | − |
| | | | nc.1 | 90916801- | 90916900- | | |
| | | | | 90916990 | 90916919 | | |
| 1471 | 84146 | ZNF644 | exon_11_c.1/ | chr1: | chr1: | AACGCUAAUCUUCCACGGAC | − |
| | | | nc.1 | 90916801- | 90916895- | | |
| | | | | 90916990 | 90916914 | | |
| 1472 | 84146 | ZNF644 | exon_11_c.1/ | chr1: | chr1: | AAUCUUCCACGGACUGGAGC | − |
| | | | nc.1 | 90916801- | 90916889- | | |
| | | | | 90916990 | 90916908 | | |
| 1473 | 84146 | ZNF644 | exon_11_nc.2 | chr1: | chr1: | CGAAGCAGCUUCAUAGAACC | − |
| | | | | 90916791- | 90916794- | | |
| | | | | 90916800 | 90916813 | | |
| 1474 | 84146 | ZNF644 | exon_11_nc.3 | chr1: | chr1: | AUUCAAACUGGCUAUUUAAA | + |
| | | | | 90916490- | 90916765- | | |
| | | | | 90916790 | 90916784 | | |
| 1475 | 84146 | ZNF644 | exon_11_nc.3 | chr1: | chr1: | UAUAAACAGAUAAUUUAAUG | + |
| | | | | 90916490- | 90916710- | | |
| | | | | 90916790 | 90916729 | | |
| 1476 | 84146 | ZNF644 | exon_11_nc.3 | chr1: | chr1: | CUGUUUAUAAAUACUAAAGC | − |
| | | | | 90916490- | 90916699- | | |
| | | | | 90916790 | 90916718 | | |
| 1477 | 84146 | ZNF644 | exon_11_nc.3 | chr1: | chr1: | AUACUAAAGCAGGAAAAUGG | − |
| | | | | 90916490- | 90916689- | | |
| | | | | 90916790 | 90916708 | | |
| 1478 | 84146 | ZNF644 | exon_11_nc.3 | chr1: | chr1: | CAUUAAUAAACUGUUGCACA | − |
| | | | | 90916490- | 90916511- | | |
| | | | | 90916790 | 90916530 | | |
| 1479 | 84146 | ZNF644 | exon_11_nc.4 | chr1: | chr1: | UUUUCUAUGCAAGUCUUGUG | + |
| | | | | 90916390- | 90916415- | | |
| | | | | 90916489 | 90916434 | | |
| 1480 | 84146 | ZNF644 | exon_11_nc.5 | chr1: | chr1: | UAAAUUUAGUCAGCGGUUCU | + |
| | | | | 90916185- | 90916345- | | |
| | | | | 90916389 | 90916364 | | |
| 1481 | 84146 | ZNF644 | exon_11_nc.5 | chr1: | chr1: | UGACUAAAUUUACUCAAAUU | − |
| | | | | 90916185- | 90916337- | | |
| | | | | 90916389 | 90916356 | | |
| 1482 | 84146 | ZNF644 | exon_11_nc.5 | chr1: | chr1: | UGAGACUGUUCACUCAAAUA | − |
| | | | | 90916185- | 90916287- | | |
| | | | | 90916389 | 90916306 | | |
| 1483 | 84146 | ZNF644 | exon_11_nc.6 | chr1: | chr1: | AUCUUUGUUGACAAAGUAGG | + |
| | | | | 90915306- | 90916056- | | |
| | | | | 90916184 | 90916075 | | |
| 1484 | 84146 | ZNF644 | exon_11_nc.6 | chr1: | chr1: | AGCAUCUUUGUUGACAAAGU | + |
| | | | | 90915306- | 90916053- | | |
| | | | | 90916184 | 90916072 | | |
| 1485 | 84146 | ZNF644 | exon_11_nc.6 | chr1: | chr1: | AUUUGACAUGCUUACAGGAC | − |
| | | | | 90915306- | 90915924- | | |
| | | | | 90916184 | 90915943 | | |
| 1486 | 84146 | ZNF644 | exon_11_nc.6 | chr1: | chr1: | CCAUACUACAUGAAAUAAAA | + |
| | | | | 90915306- | 90915490- | | |
| | | | | 90916184 | 90915509 | | |
| 1487 | 84146 | ZNF644 | exon_11_nc.6 | chr1: | chr1: | CUAAUACUAUAGUUGAACCA | − |
| | | | | 90915306- | 90915470- | | |
| | | | | 90916184 | 90915489 | | |
| 1488 | 84146 | ZNF644 | exon_11_nc.6 | chr1: | chr1: | CUGGUAAGGAGAUAUACCAU | + |
| | | | | 90915306- | 90915338- | | |
| | | | | 90916184 | 90915357 | | |

TABLE 2

| | target | target_ | target_region_ | target_region_ | gRNA_target_ | | |
|---|---|---|---|---|---|---|---|
| SEQ ID NO | gene_id | symbol | name | coordinates | site_coordinates | gRNA Targeting Domain | strand |
| 1489 | 84146 | ZNF644 | promoter1.2 | chr1: 91022273-91023899 | chr1: 91023879-91023898 | CUCUGUCUAGCCGCAGACGU | + |
| 1490 | 84146 | ZNF644 | promoter1.2 | chr1: 91022273-91023899 | chr1: 91023790-91023809 | AUCUCAAAAGCUUCUAUGAA | + |
| 1491 | 84146 | ZNF644 | promoter1.2 | chr1: 91022273-91023899 | chr1: 91023686-91023705 | GCAACUGAAUUGUAAUACUG | + |
| 1492 | 84146 | ZNF644 | promoter1.2 | chr1: 91022273-91023899 | chr1: 91023621-91023640 | UUCAGAACUCAGCCAUGAUC | + |
| 1493 | 84146 | ZNF644 | promoter1.2 | chr1: 91022273-91023899 | chr1: 91023456-91023475 | CUGAGAAUGUCCAAUUUUAA | − |
| 1494 | 84146 | ZNF644 | promoter1.2 | chr1: 91022273-91023899 | chr1: 91023401-91023420 | AUUUGCAGUCAAUGGGGGAG | + |
| 1495 | 84146 | ZNF644 | promoter1.2 | chr1: 91022273-91023899 | chr1: 91023396-91023415 | UAUAUAUUUGCAGUCAAUGG | + |
| 1496 | 84146 | ZNF644 | promoter1.2 | chr1: 91022273-91023899 | chr1: 91023353-91023372 | ACAUUUGUGUAAAUGAAGAC | − |
| 1497 | 84146 | ZNF644 | promoter1.2 | chr1: 91022273-91023899 | chr1: 91023307-91023326 | CUCGAAGUCAUACAGUUCUU | + |
| 1498 | 84146 | ZNF644 | promoter1.2 | chr1: 91022273-91023899 | chr1: 91023277-91023296 | CAAUGAGACAAAGAGGAGUA | + |
| 1499 | 84146 | ZNF644 | promoter1.2 | chr1: 91022273-91023899 | chr1: 91023279-91023298 | CUUACUCCUCUUUGUCUCAU | − |
| 1500 | 84146 | ZNF644 | promoter1.2 | chr1: 91022273-91023899 | chr1: 91023278-91023297 | UUACUCCUCUUUGUCUCAUU | − |
| 1501 | 84146 | ZNF644 | promoter1.2 | chr1: 91022273-91023899 | chr1: 91023164-91023183 | UUAGUCAAAUAUUUGUUGUU | − |
| 1502 | 84146 | ZNF644 | promoter1.2 | chr1: 91022273-91023899 | chr1: 91023139-91023158 | UCUACGUUUGCUGUUGCUGA | − |
| 1503 | 84146 | ZNF644 | promoter1.2 | chr1: 91022273-91023899 | chr1: 91023047-91023066 | CUCAAGAUGUUAUUGACAUU | + |
| 1504 | 84146 | ZNF644 | promoter1.2 | chr1: 91022273-91023899 | chr1: 91023040-91023059 | AUAACAUCUUGAGGGUUUGG | − |
| 1505 | 84146 | ZNF644 | promoter1.2 | chr1: 91022273-91023899 | chr1: 91023010-91023029 | UAAUGACAGCUGUACAACUA | − |
| 1506 | 84146 | ZNF644 | promoter1.2 | chr1: 91022273-91023899 | chr1: 91022913-91022932 | AGAGUUCUUGAGAAAUCUAC | + |
| 1507 | 84146 | ZNF644 | promoter1.2 | chr1: 91022273-91023899 | chr1: 91022830-91022849 | ACUCAGUGUUUCUCUGCCCC | − |
| 1508 | 84146 | ZNF644 | promoter1.2 | chr1: 91022273-91023899 | chr1: 91022809-91022828 | AAGACUUCACCACAGGAUCC | + |
| 1509 | 84146 | ZNF644 | promoter1.2 | chr1: 91022273-91023899 | chr1: 91022802-91022821 | UUUUAACAAGACUUCACCAC | + |
| 1510 | 84146 | ZNF644 | promoter1.2 | chr1: 91022273-91023899 | chr1: 91022779-91022798 | AAAAUUUCCAGAACCCUCUA | − |
| 1511 | 84146 | ZNF644 | promoter1.2 | chr1: 91022273-91023899 | chr1: 91022732-91022751 | AUAAGAAGCAUGUGAGAUGC | + |
| 1512 | 84146 | ZNF644 | promoter1.2 | chr1: 91022273-91023899 | chr1: 91022690-91022709 | CUGCAUUACUGAAAGUGUCG | + |
| 1513 | 84146 | ZNF644 | promoter1.2 | chr1: 91022273-91023899 | chr1: 91022656-91022675 | UGCUCCAGUUUGACUCUCUG | + |
| 1514 | 84146 | ZNF644 | promoter1.2 | chr1: 91022273-91023899 | chr1: 91022639-91022658 | GCAAACCUCGGCCACCUUUC | − |
| 1515 | 84146 | ZNF644 | promoter1.2 | chr1: 91022273-91023899 | chr1: 91022638-91022657 | CAAACCUCGGCCACCUUUCA | − |
| 1516 | 84146 | ZNF644 | promoter1.2 | chr1: 91022273-91023899 | chr1: 91022592-91022611 | GUCACACUUGAUGCUUUGCC | − |
| 1517 | 84146 | ZNF644 | promoter1.2 | chr1: 91022273-91023899 | chr1: 91022517-91022536 | CAACUGUGGAGUAAUGGCAG | + |
| 1518 | 84146 | ZNF644 | promoter1.2 | chr1: 91022273-91023899 | chr1: 91022503-91022522 | AUAUUUGCCAAUCACAACUG | + |
| 1519 | 84146 | ZNF644 | promoter1.2 | chr1: 91022273-91023899 | chr1: 91022435-91022454 | GUGAUCGUGGAACUCAAACC | − |
| 1520 | 84146 | ZNF644 | promoter1.2 | chr1: 91022273-91023899 | chr1: 91022434-91022453 | UGAUCGUGGAACUCAAACCU | − |
| 1521 | 84146 | ZNF644 | promoter1.2 | chr1: 91022273-91023899 | chr1: 91022389-91022408 | CCCAAGCACUAGGCAUGCCU | + |
| 1522 | 84146 | ZNF644 | promoter1.2 | chr1: 91022273-91023899 | chr1: 91022393-91022412 | GCCUAGGCAUGCCUAGUGCU | − |
| 1523 | 84146 | ZNF644 | promoter1.2 | chr1: 91022273-91023899 | chr1: 91022392-91022411 | CCUAGGCAUGCCUAGUGCUU | − |
| 1524 | 84146 | ZNF644 | promoter1.2 | chr1: 91022273-91023899 | chr1: 91022376-91022395 | GCUUGGGUCUUGCAGAGUUU | − |
| 1525 | 84146 | ZNF644 | promoter1.2 | chr1: 91022273-91023899 | chr1: 91022361-91022380 | AGUUUGGGCACCACCAUAUU | − |
| 1526 | 84146 | ZNF644 | promoter1.2 | chr1: 91022273-91023899 | chr1: 91022311-91022330 | UGACCUAAUCACUCCCAAGG | + |
| 1527 | 84146 | ZNF644 | promoter1.2 | chr1: 91022273-91023899 | chr1: 91022305-91022324 | GAGUGAUUAGGUCAGCGCUG | − |
| 1528 | 84146 | ZNF644 | promoter1.2 | chr1: 91022273-91023899 | chr1: 91022304-91022323 | AGUGAUUAGGUCAGCGCUGG | − |
| 1529 | 84146 | ZNF644 | promoter1.2 | chr1: 91022273-91023899 | chr1: 91022301-91022320 | GAUUAGGUCAGCGCUGGGGG | − |
| 1530 | 84146 | ZNF644 | exon_01_nc.1 | chr1: 91022256-91022272 | chr1: 91022253-91022272 | AACAGUGCACCACUGUUCUC | + |
| 1531 | 84146 | ZNF644 | exon_01_nc.2 | chr1: 91022082-91022255 | chr1: 91022230-91022249 | GCUGCCCUCGUUUGUCUAAU | − |
| 1532 | 84146 | ZNF644 | exon_01_nc.2 | chr1: 91022082-91022255 | chr1: 91022172-91022191 | CUCCUCGCUGGCCACCCCCG | − |
| 1533 | 84146 | ZNF644 | exon_01_nc.2 | chr1: 91022082-91022255 | chr1: 91022145-91022164 | UUCCCGCCUCUGCCGGGCUG | + |
| 1534 | 84146 | ZNF644 | exon_01_nc.2 | chr1: 91022082-91022255 | chr1: 91022151-91022170 | GGCCCGCAGCCCGGCAGAGG | − |
| 1535 | 84146 | ZNF644 | exon_01_nc.2 | chr1: 91022082-91022255 | chr1: 91022127-91022146 | AAAGCAGCGCGCCUGCGCCG | + |
| 1536 | 84146 | ZNF644 | exon_01_nc.2 | chr1: 91022082-91022255 | chr1: 91022113-91022132 | AGCCCCGGUUCCCACGGCGC | + |
| 1537 | 84146 | ZNF644 | exon_01_nc.2 | chr1: 91022082-91022255 | chr1: 91022126-91022145 | AAGCAGCGCGCCUGCGCCGU | − |
| 1538 | 84146 | ZNF644 | exon_01_nc.2 | chr1: 91022082-91022255 | chr1: 91022114-91022133 | UGCGCCGUGGGAACCGGGGC | + |
| 1539 | 84146 | ZNF644 | exon_01_nc.2 | chr1: 91022082-91022255 | chr1: 91022098-91022117 | CCGCUUUUCGCCUCCAGCCC | + |
| 1540 | 84146 | ZNF644 | exon_01_nc.2 | chr1: 91022082-91022255 | chr1: 91022111-91022130 | GCCGUGGGAACCGGGGCUGG | − |
| 1541 | 84146 | ZNF644 | exon_01_nc.2 | chr1: 91022082-91022255 | chr1: 91022101-91022120 | CCGGGGCUGGAGGCGAAAAG | − |
| 1542 | 84146 | ZNF644 | exon_01_nc.2 | chr1: 91022082-91022255 | chr1: 91022094-91022113 | UGGAGGCGAAAAGCGGGGAG | − |
| 1543 | 84146 | ZNF644 | exon_01_nc.2 | chr1: 91022082-91022255 | chr1: 91022091-91022110 | AGGCGAAAAGCGGGGAGCGG | − |
| 1544 | 84146 | ZNF644 | exon_01_nc.2 | chr1: 91022082-91022255 | chr1: 91022090-91022109 | GGCGAAAAGCGGGGAGCGGA | − |
| 1545 | 84146 | ZNF644 | exon_01_nc.2 | chr1: 91022082-91022255 | chr1: 91022089-91022108 | GCGAAAAGCGGGGAGCGGAG | − |
| 1546 | 84146 | ZNF644 | exon_01_nc.2 | chr1: 91022082-91022255 | chr1: 91022088-91022107 | CGAAAAGCGGGGAGCGGAGG | − |
| 1547 | 84146 | ZNF644 | exon_01_nc.4 | chr1: 91022036-91022055 | chr1: 91022046-91022065 | GUACAGAGCGGCGUGUGACG | − |
| 1548 | 84146 | ZNF644 | exon_01_nc.5 | chr1: 91022015-91022035 | chr1: 91022015-91022034 | GGGGCGACGUUGGGAGCACG | + |
| 1549 | 84146 | ZNF644 | intron_01 | chr1: 91021726-91021989 | chr1: 91021980-91021999 | GCACCAAACUGUGAGUUUCA | − |
| 1550 | 84146 | ZNF644 | intron_01 | chr1: 91021726-91021989 | chr1: 91021947-91021966 | CAGAUUCGCUGGGACCCGAG | − |
| 1551 | 84146 | ZNF644 | intron_01 | chr1: 91021726-91021989 | chr1: 91021930-91021949 | CAGAGGCCCGAAACCCACUC | + |
| 1552 | 84146 | ZNF644 | intron_01 | chr1: 91021726-91021989 | chr1: 91021940-91021959 | GCUGGGACCCGAGUGGGUUU | − |
| 1553 | 84146 | ZNF644 | intron_01 | chr1: 91021726-91021989 | chr1: 91021907-91021926 | UGUGGCAGCGGCGGGACUGA | − |
| 1554 | 84146 | ZNF644 | intron_01 | chr1: 91021726-91021989 | chr1: 91021888-91021907 | AGGGUGGCGGAAGAACUUGG | − |
| 1555 | 84146 | ZNF644 | intron_01 | chr1: 91021726-91021989 | chr1: 91021882-91021901 | GCGGAAGAACUUGGGGGACC | − |
| 1556 | 84146 | ZNF644 | intron_01 | chr1: 91021726-91021989 | chr1: 91021856-91021875 | UCCCCGGGCCCGAGAGCCGG | + |
| 1557 | 84146 | ZNF644 | intron_01 | chr1: 91021726-91021989 | chr1: 91021868-91021887 | GGGACCCGGCCUCCGGCUCU | − |
| 1558 | 84146 | ZNF644 | intron_01 | chr1: 91021726-91021989 | chr1: 91021867-91021886 | GGACCCGGCCUCCGGCUCUC | − |
| 1559 | 84146 | ZNF644 | intron_01 | chr1: 91021726-91021989 | chr1: 91021862-91021881 | CGGCCUCCGGCUCUCGGGCC | − |
| 1560 | 84146 | ZNF644 | intron_01 | chr1: 91021726-91021989 | chr1: 91021841-91021860 | AGCCUAGGGCGUAGCUCCCC | + |
| 1561 | 84146 | ZNF644 | intron_01 | chr1: 91021726-91021989 | chr1: 91021840-91021859 | CAGCCUAGGGCGUAGCUCCC | + |
| 1562 | 84146 | ZNF644 | intron_01 | chr1: 91021726-91021989 | chr1: 91021846-91021865 | GGCCCGGGGAGCUACGCCCU | − |
| 1563 | 84146 | ZNF644 | intron_01 | chr1: 91021726-91021989 | chr1: 91021827-91021846 | GAUGGCUGGGACCCAGCCUA | + |
| 1564 | 84146 | ZNF644 | intron_01 | chr1: 91021726-91021989 | chr1: 91021787-91021806 | CGCCGCCAUCUUAGCCUGUG | + |
| 1565 | 84146 | ZNF644 | intron_01 | chr1: 91021726-91021989 | chr1: 91021786-91021805 | ACAGGCUAAGAUGGCGGCGG | − |
| 1566 | 84146 | ZNF644 | intron_01 | chr1: 91021726-91021989 | chr1: 91021749-91021768 | UUGGUGCUACACAAGGAGCC | − |
| 1567 | 84146 | ZNF644 | intron_01 | chr1: 91021726-91021989 | chr1: 91021748-91021767 | UGGUGCUACACAAGGAGCCC | − |
| 1568 | 84146 | ZNF644 | intron_01 | chr1: 91021726-91021989 | chr1: 91021741-91021760 | ACACAAGGAGCCCGGGCCCC | − |
| 1569 | 84146 | ZNF644 | intron_01 | chr1: 91021726-91021989 | chr1: 91021735-91021754 | GGAGCCCGGGCCCCGGGUUC | − |
| 1570 | 84146 | ZNF644 | intron_01 | chr1: 91021726-91021989 | chr1: 91021720-91021739 | GGCCGCCGCCUCCCCGAACC | + |
| 1571 | 84146 | ZNF644 | intron_01 | chr1: 91021726-91021989 | chr1: 91021731-91021750 | CCCGGGCCCCGGGUUCGGGG | − |
| 1572 | 84146 | ZNF644 | exon_02_nc | chr1: 91021689-91021725 | chr1: 91021719-91021738 | GUUCGGGGAGGCGGCGGCCG | − |
| 1573 | 84146 | ZNF644 | intron_02 | chr1: 91021509-91021688 | chr1: 91021655-91021674 | CGAGUCCCGCCCCGCGGCGG | + |
| 1574 | 84146 | ZNF644 | intron_02 | chr1: 91021509-91021688 | chr1: 91021652-91021671 | CGCCGAGUCCCGCCCCGCGG | + |
| 1575 | 84146 | ZNF644 | intron_02 | chr1: 91021509-91021688 | chr1: 91021645-91021664 | GCGGGACUCGGCGGGGGGAU | − |
| 1576 | 84146 | ZNF644 | intron_02 | chr1: 91021509-91021688 | chr1: 91021625-91021644 | GGGGGGGGGGCUUUCCGCUG | − |
| 1577 | 84146 | ZNF644 | intron_02 | chr1: 91021509-91021688 | chr1: 91021610-91021629 | CGCUGCGGCGUCGGAGGAUC | − |
| 1578 | 84146 | ZNF644 | intron_02 | chr1: 91021509-91021688 | chr1: 91021523-91021542 | GAGCAGCCAGUGAGCAGUAG | − |
| 1579 | 84146 | ZNF644 | intron_02 | chr1: 91021509-91021688 | chr1: 91021517-91021536 | CCAGUGAGCAGUAGGGGGAA | − |
| 1580 | 84146 | ZNF644 | exon_03_nc.1 | chr1: 91021474-91021508 | chr1: 91021488-91021507 | AUCAGGCGGCUUGGGGAGCC | − |
| 1581 | 84146 | ZNF644 | exon_03_nc.2 | chr1: 91021458-91021473 | chr1: 91021457-91021476 | AUUGCAAUGCACUGAGUAAC | − |
| 1582 | 84146 | ZNF644 | exon_03_nc.3 | chr1: 91021273-91021457 | chr1: 91021380-91021399 | AAUGCCAGAGCCAGCGUUUC | + |
| 1583 | 84146 | ZNF644 | exon_03_nc.3 | chr1: 91021273-91021457 | chr1: 91021387-91021406 | GUGGCCAGAAACGCUGGCUC | − |
| 1584 | 84146 | ZNF644 | exon_03_nc.3 | chr1: 91021273-91021457 | chr1: 91021341-91021360 | GCGGUCCUGUCCCAAGUAAG | + |
| 1585 | 84146 | ZNF644 | exon_03_nc.3 | chr1: 91021273-91021457 | chr1: 91021355-91021374 | UCUACACCCCUCCCCUUACU | − |
| 1586 | 84146 | ZNF644 | exon_03_nc.3 | chr1: 91021273-91021457 | chr1: 91021308-91021327 | AGAUCUUGCAGGACGGGGGG | + |
| 1587 | 84146 | ZNF644 | exon_03_nc.3 | chr1: 91021273-91021457 | chr1: 91021275-91021294 | GCCUGGGAAGUAACAGCAAG | − |
| 1588 | 84146 | ZNF644 | intron_03 | chr1: 91021202-91021272 | chr1: 91021204-91021223 | CUAAAGCUUUUUGCCUCUCC | − |
| 1589 | 84146 | ZNF644 | intron_03 | chr1: 91021202-91021272 | chr1: 91021188-91021207 | CCAGAGAAUCAGACCUGGAG | + |
| 1590 | 84146 | ZNF644 | exon_04_nc.1 | chr1: 91020529-91021201 | chr1: 91021157-91021176 | CUGGGAAGUUACUCUGCCAG | − |
| 1591 | 84146 | ZNF644 | exon_04_nc.1 | chr1: 91020529-91021201 | chr1: 91021149-91021168 | UUACUCUGCCAGAGGAAAGC | − |
| 1592 | 84146 | ZNF644 | exon_04_nc.1 | chr1: 91020529-91021201 | chr1: 91021109-91021128 | CAACUCAUCUUCAGGUUCAC | + |
| 1593 | 84146 | ZNF644 | exon_04_nc.1 | chr1: 91020529-91021201 | chr1: 91021079-91021098 | UUCUCAGUGAUUGGAAACUU | − |
| 1594 | 84146 | ZNF644 | exon_04_nc.1 | chr1: 91020529-91021201 | chr1: 91021076-91021095 | UCAGUGAUUGGAAACUUAGG | − |
| 1595 | 84146 | ZNF644 | exon_04_nc.1 | chr1: 91020529-91021201 | chr1: 91021073-91021092 | GUGAUUGGAAACUUAGGUGG | − |
| 1596 | 84146 | ZNF644 | exon_04_nc.1 | chr1: 91020529-91021201 | chr1: 91021026-91021045 | CUUCUACAGACAACUCUUGU | − |
| 1597 | 84146 | ZNF644 | exon_04_nc.1 | chr1: 91020529-91021201 | chr1: 91021025-91021044 | UUCUACAGACAACUCUUGUU | − |
| 1598 | 84146 | ZNF644 | exon_04_nc.1 | chr1: 91020529-91021201 | chr1: 91020909-91020928 | UGAUCAUCAAACUCAACGAG | − |
| 1599 | 84146 | ZNF644 | exon_04_nc.1 | chr1: 91020529-91021201 | chr1: 91020814-91020833 | UUAGGACUGGACAGGAUAGU | + |
| 1600 | 84146 | ZNF644 | exon_04_nc.1 | chr1: 91020529-91021201 | chr1: 91020739-91020758 | AACUAAAGCAAACUACUGCA | + |
| 1601 | 84146 | ZNF644 | intron_04 | chr1: 90982371-91020447 | chr1: 91020420-91020439 | AAUUAUAUCAUACUUUGUGU | − |
| 1602 | 84146 | ZNF644 | intron_04 | chr1: 90982371-91020447 | chr1: 91020346-91020365 | AAUUAACAGAAUUCCUUGGU | − |
| 1603 | 84146 | ZNF644 | intron_04 | chr1: 90982371-91020447 | chr1: 91020330-91020349 | UUUAAAUAUCCCUCCAACCA | + |
| 1604 | 84146 | ZNF644 | intron_04 | chr1: 90982371-91020447 | chr1: 91020343-91020362 | UAACAGAAUUCCUUGGUUGG | − |
| 1605 | 84146 | ZNF644 | intron_04 | chr1: 90982371-91020447 | chr1: 91020342-91020361 | AACAGAAUUCCUUGGUUGGA | − |
| 1606 | 84146 | ZNF644 | intron_04 | chr1: 90982371-91020447 | chr1: 91020133-91020152 | UGGAUCAUAAAGUUUUACAG | − |
| 1607 | 84146 | ZNF644 | intron_04 | chr1: 90982371-91020447 | chr1: 91020075-91020094 | UCCUCAAUAUAUUGUAAAAG | + |
| 1608 | 84146 | ZNF644 | intron_04 | chr1: 90982371-91020447 | chr1: 91020052-91020071 | GCCACUAACUUAAUCAUCUC | + |
| 1609 | 84146 | ZNF644 | intron_04 | chr1: 90982371-91020447 | chr1: 91020000-91020019 | AUGACAAAUACAAACAGCAA | + |
| 1610 | 84146 | ZNF644 | intron_04 | chr1: 90982371-91020447 | chr1: 91019985-91020004 | GUCAUAUUGCUGGGAUGAGA | − |
| 1611 | 84146 | ZNF644 | intron_04 | chr1: 90982371-91020447 | chr1: 91019951-91019970 | AACUAAUGGUUAAAAAAACA | − |
| 1612 | 84146 | ZNF644 | intron_04 | chr1: 90982371-91020447 | chr1: 91019548-91019567 | UUCAUAUUCUGUGGUUAACU | − |
| 1613 | 84146 | ZNF644 | intron_04 | chr1: 90982371-91020447 | chr1: 91019514-91019533 | ACUUUCAGUGAAACAAAAUC | − |
| 1614 | 84146 | ZNF644 | intron_04 | chr1: 90982371-91020447 | chr1: 91019381-91019400 | AAAGAAAUUUAUCAAGUCCC | + |
| 1615 | 84146 | ZNF644 | intron_04 | chr1: 90982371-91020447 | chr1: 91019331-91019350 | UUCUUUUCCUACAUAAUAAC | + |
| 1616 | 84146 | ZNF644 | intron_04 | chr1: 90982371-91020447 | chr1: 91019175-91019194 | AAUUUCCUAACAGACCUUAG | + |
| 1617 | 84146 | ZNF644 | intron_04 | chr1: 90982371-91020447 | chr1: 91018988-91019007 | AAUUUUCAACAUUAUGUGAG | − |
| 1618 | 84146 | ZNF644 | intron_04 | chr1: 90982371-91020447 | chr1: 91018922-91018941 | GGUUAUUUGUAGUUAUAAAG | − |
| 1619 | 84146 | ZNF644 | intron_04 | chr1: 90982371-91020447 | chr1: 91018854-91018873 | UUCAAGUUAUAAGAGUGUUU | − |
| 1620 | 84146 | ZNF644 | intron_04 | chr1: 90982371-91020447 | chr1: 91018833-91018852 | GGUUGAAUUGAGACUGUUAA | − |
| 1621 | 84146 | ZNF644 | intron_04 | chr1: 90982371-91020447 | chr1: 91018832-91018851 | GUUGAAUUGAGACUGUUAAU | − |
| 1622 | 84146 | ZNF644 | intron_04 | chr1: 90982371-91020447 | chr1: 91018822-91018841 | GACUGUUAAUGGGUAGCAGG | − |
| 1623 | 84146 | ZNF644 | intron_04 | chr1: 90982371-91020447 | chr1: 91018786-91018805 | UCAAAGAGAAUAGAAGUGGU | − |
| 1624 | 84146 | ZNF644 | intron_04 | chr1: 90982371-91020447 | chr1: 91018764-91018783 | GAUAUCUGCAAUCUAGAGAA | − |
| 1625 | 84146 | ZNF644 | intron_04 | chr1: 90982371-91020447 | chr1: 91018759-91018778 | CUGCAAUCUAGAGAAAGGUC | − |
| 1626 | 84146 | ZNF644 | intron_04 | chr1: 90982371-91020447 | chr1: 91018680-91018699 | AGAAUCACAUCCUGAGAAAA | + |
| 1627 | 84146 | ZNF644 | intron_04 | chr1: 90982371-91020447 | chr1: 91018677-91018696 | UCUCAGGAUGUGAUUCUGGA | − |
| 1628 | 84146 | ZNF644 | intron_04 | chr1: 90982371-91020447 | chr1: 91018598-91018617 | CUUGCAGCAAAUGGAGAAAU | − |
| 1629 | 84146 | ZNF644 | intron_04 | chr1: 90982371-91020447 | chr1: 91018419-91018438 | UUUACACUUAUUAAUCUUUC | − |
| 1630 | 84146 | ZNF644 | intron_04 | chr1: 90982371-91020447 | chr1: 91018392-91018411 | GAGAAUGUCUAUUUCGUAUA | − |
| 1631 | 84146 | ZNF644 | intron_04 | chr1: 90982371-91020447 | chr1: 91018328-91018347 | GACUGAAGCAGUUCUAGAAG | − |
| 1632 | 84146 | ZNF644 | intron_04 | chr1: 90982371-91020447 | chr1: 91018246-91018265 | CCAAAACUCAAUGGUGUAAA | + |
| 1633 | 84146 | ZNF644 | intron_04 | chr1: 90982371-91020447 | chr1: 91018134-91018153 | AAGAUCACAGGAGACCCAGA | − |
| 1634 | 84146 | ZNF644 | intron_04 | chr1: 90982371-91020447 | chr1: 91018060-91018079 | CAAGAAAACACCUUUUUCAU | + |
| 1635 | 84146 | ZNF644 | intron_04 | chr1: 90982371-91020447 | chr1: 91018073-91018092 | AGAGGAAGUUCCCAUGAAAA | − |
| 1636 | 84146 | ZNF644 | intron_04 | chr1: 90982371-91020447 | chr1: 91018054-91018073 | AAGGUGUUUUCUUGUCUCUA | − |
| 1637 | 84146 | ZNF644 | intron_04 | chr1: 90982371-91020447 | chr1: 91018001-91018020 | AGAUCUGCCAAGUUGAACCA | − |
| 1638 | 84146 | ZNF644 | intron_04 | chr1: 90982371-91020447 | chr1: 91017653-91017672 | CUAUCCUUUUUGAACUUCCU | − |
| 1639 | 84146 | ZNF644 | intron_04 | chr1: 90982371-91020447 | chr1: 91017633-91017652 | AUAAUCACAUAAGAGUUCCC | + |
| 1640 | 84146 | ZNF644 | intron_04 | chr1: 90982371-91020447 | chr1: 91017565-91017584 | UAACAUCUCUCAAUUGUCAA | + |
| 1641 | 84146 | ZNF644 | intron_04 | chr1: 90982371-91020447 | chr1: 91017430-91017449 | UGGUUGGGAGAAAGUGAGAC | − |
| 1642 | 84146 | ZNF644 | intron_04 | chr1: 90982371-91020447 | chr1: 91017372-91017391 | GGCCAAGAUUAGUAGGAGAG | + |
| 1643 | 84146 | ZNF644 | intron_04 | chr1: 90982371-91020447 | chr1: 91017371-91017390 | GGGCCAAGAUUAGUAGGAGA | + |
| 1644 | 84146 | ZNF644 | intron_04 | chr1: 90982371-91020447 | chr1: 91017377-91017396 | UUCCCCUCUCCUACUAAUCU | − |
| 1645 | 84146 | ZNF644 | intron_04 | chr1: 90982371-91020447 | chr1: 91017351-91017370 | GCUAGCAGACAGAAGAGGUA | + |
| 1646 | 84146 | ZNF644 | intron_04 | chr1: 90982371-91020447 | chr1: 91017319-91017338 | CAAAGGAUUCCAAAUGAAAU | + |
| 1647 | 84146 | ZNF644 | intron_04 | chr1: 90982371-91020447 | chr1: 91017318-91017337 | UCAAAGGAUUCCAAAUGAAA | + |
| 1648 | 84146 | ZNF644 | intron_04 | chr1: 90982371-91020447 | chr1: 91017246-91017265 | AAUUCUGUUAGCCUAGAUUU | − |
| 1649 | 84146 | ZNF644 | intron_04 | chr1: 90982371-91020447 | chr1: 91017231-91017250 | GAUUUUGGUAGAAUUGAAGC | − |
| 1650 | 84146 | ZNF644 | intron_04 | chr1: 90982371-91020447 | chr1: 91016700-91016719 | UAAAAUAAUUGAUAGACUAC | + |
| 1651 | 84146 | ZNF644 | intron_04 | chr1: 90982371-91020447 | chr1: 91016244-91016263 | CUUUCAGAGCACCAUUUAUU | − |
| 1652 | 84146 | ZNF644 | intron_04 | chr1: 90982371-91020447 | chr1: 91016240-91016259 | CAGAGCACCAUUUAUUGGGG | − |
| 1653 | 84146 | ZNF644 | intron_04 | chr1: 90982371-91020447 | chr1: 91016233-91016252 | CCAUUUAUUGGGGAGGAGAG | − |
| 1654 | 84146 | ZNF644 | intron_04 | chr1: 90982371-91020447 | chr1: 91016201-91016220 | UUCACAAACCAACAAUAAAU | + |
| 1655 | 84146 | ZNF644 | intron_04 | chr1: 90982371-91020447 | chr1: 91016202-91016221 | AAUUUAUUGUUGGUUUGUGA | − |
| 1656 | 84146 | ZNF644 | intron_04 | chr1: 90982371-91020447 | chr1: 91016191-91016210 | GGUUUGUGAAGGUGGGUAAA | − |
| 1657 | 84146 | ZNF644 | intron_04 | chr1: 90982371-91020447 | chr1: 91016142-91016161 | GUGAGUCAGUAGCAAACAAA | + |
| 1658 | 84146 | ZNF644 | intron_04 | chr1: 90982371-91020447 | chr1: 91016017-91016036 | ACCCUGUCAAUUUUACUUCA | + |
| 1659 | 84146 | ZNF644 | intron_04 | chr1: 90982371-91020447 | chr1: 91016021-91016040 | GCCUUGAAGUAAAAUUGACA | − |
| 1660 | 84146 | ZNF644 | intron_04 | chr1: 90982371-91020447 | chr1: 91015949-91015968 | ACCCUAAACCUCUUUGAAGA | + |
| 1661 | 84146 | ZNF644 | intron_04 | chr1: 90982371-91020447 | chr1: 91015901-91015920 | CAUUGUAUGCCAGAAAGCUA | + |
| 1662 | 84146 | ZNF644 | intron_04 | chr1: 90982371-91020447 | chr1: 91015789-91015808 | UACAACUGCAAAUCACUCAC | + |
| 1663 | 84146 | ZNF644 | intron_04 | chr1: 90982371-91020447 | chr1: 91015751-91015770 | UUAUCGGCAUGGCUUAAAAU | + |
| 1664 | 84146 | ZNF644 | intron_04 | chr1: 90982371-91020447 | chr1: 91015750-91015769 | UUUAUCGGCAUGGCUUAAAA | + |
| 1665 | 84146 | ZNF644 | intron_04 | chr1: 90982371-91020447 | chr1: 91015734-91015753 | UAAAGACAAGGUUGUUAUAG | − |
| 1666 | 84146 | ZNF644 | intron_04 | chr1: 90982371-91020447 | chr1: 91015650-91015669 | CAUUCCUGAAAUAUUUAACC | + |
| 1667 | 84146 | ZNF644 | intron_04 | chr1: 90982371-91020447 | chr1: 91015657-91015676 | AUAACCAGGUUAAAUAUUUC | − |
| 1668 | 84146 | ZNF644 | intron_04 | chr1: 90982371-91020447 | chr1: 91015622-91015641 | UACUCAUCUGCUGACAGUGC | + |
| 1669 | 84146 | ZNF644 | intron_04 | chr1: 90982371-91020447 | chr1: 91015613-91015632 | GCAGAUGAGUAGAUACACUU | − |
| 1670 | 84146 | ZNF644 | intron_04 | chr1: 90982371-91020447 | chr1: 91015559-91015578 | AGACAAACCAAUGCACUGGC | + |
| 1671 | 84146 | ZNF644 | intron_04 | chr1: 90982371-91020447 | chr1: 91015555-91015574 | ACAAAGACAAACCAAUGCAC | + |
| 1672 | 84146 | ZNF644 | intron_04 | chr1: 90982371-91020447 | chr1: 91015569-91015588 | UUAUUUCCCAGCCAGUGCAU | − |
| 1673 | 84146 | ZNF644 | intron_04 | chr1: 90982371-91020447 | chr1: 91015506-91015525 | CCUCACAGUCAAAAAUUGAA | + |
| 1674 | 84146 | ZNF644 | intron_04 | chr1: 90982371-91020447 | chr1: 91015509-91015528 | CCUUUCAAUUUUUGACUGUG | − |
| 1675 | 84146 | ZNF644 | intron_04 | chr1: 90982371-91020447 | chr1: 91015501-91015520 | UUUUUGACUGUGAGGCCAUG | − |
| 1676 | 84146 | ZNF644 | intron_04 | chr1: 90982371-91020447 | chr1: 91015357-91015376 | UUUCAUUGGAUAGUCUGAGU | − |
| 1677 | 84146 | ZNF644 | intron_04 | chr1: 90982371-91020447 | chr1: 91015356-91015375 | UUCAUUGGAUAGUCUGAGUU | − |
| 1678 | 84146 | ZNF644 | intron_04 | chr1: 90982371-91020447 | chr1: 91015287-91015306 | UGUUUUGUAUAUGUCUGUGC | − |
| 1679 | 84146 | ZNF644 | intron_04 | chr1: 90982371-91020447 | chr1: 91015216-91015235 | UUGUGCUAGGAGGAGGUUGG | + |
| 1680 | 84146 | ZNF644 | intron_04 | chr1: 90982371-91020447 | chr1: 91015203-91015222 | UUAAAAUCCGUGUUUGUGCU | + |
| 1681 | 84146 | ZNF644 | intron_04 | chr1: 90982371-91020447 | chr1: 91015145-91015164 | AAUUUUAGGUUCAUUUGAGG | − |
| 1682 | 84146 | ZNF644 | intron_04 | chr1: 90982371-91020447 | chr1: 91015143-91015162 | UUUUAGGUUCAUUUGAGGAG | − |
| 1683 | 84146 | ZNF644 | intron_04 | chr1: 90982371-91020447 | chr1: 91015088-91015107 | GCUUGAAAAAUACAGCAAUU | − |
| 1684 | 84146 | ZNF644 | intron_04 | chr1: 90982371-91020447 | chr1: 91015074-91015093 | GCAAUUUGGAAAGUGAGGGA | − |
| 1685 | 84146 | ZNF644 | intron_04 | chr1: 90982371-91020447 | chr1: 91014904-91014923 | GAGUAGCCAAGUUUGAACCU | − |
| 1686 | 84146 | ZNF644 | intron_04 | chr1: 90982371-91020447 | chr1: 91014804-91014823 | UUACAAAUUGUGUAACAACU | + |
| 1687 | 84146 | ZNF644 | intron_04 | chr1: 90982371-91020447 | chr1: 91014684-91014703 | GACAAAGAGCCUUUAUAUGG | + |
| 1688 | 84146 | ZNF644 | intron_04 | chr1: 90982371-91020447 | chr1: 91014681-91014700 | CAAGACAAAGAGCCUUUAUA | + |
| 1689 | 84146 | ZNF644 | intron_04 | chr1: 90982371-91020447 | chr1: 91014669-91014688 | UUGUCUUGGCUGCUGGACAU | − |
| 1690 | 84146 | ZNF644 | intron_04 | chr1: 90982371-91020447 | chr1: 91014562-91014581 | UAUGGGAUUUGGAUUGCUAC | − |
| 1691 | 84146 | ZNF644 | intron_04 | chr1: 90982371-91020447 | chr1: 91014518-91014537 | AGUCAAAUGUCCCACCAGCA | + |
| 1692 | 84146 | ZNF644 | intron_04 | chr1: 90982371-91020447 | chr1: 91014391-91014410 | UCCCUUUAAGAUUUUUAACA | + |
| 1693 | 84146 | ZNF644 | intron_04 | chr1: 90982371-91020447 | chr1: 91013735-91013754 | GCUACUGAAUACCUGAACUC | + |
| 1694 | 84146 | ZNF644 | intron_04 | chr1: 90982371-91020447 | chr1: 91013624-91013643 | CUGUUCUCAGCUAUCCUUUA | + |
| 1695 | 84146 | ZNF644 | intron_04 | chr1: 90982371-91020447 | chr1: 91013526-91013545 | AGUGGCUUGAAUCCAGUUUA | − |
| 1696 | 84146 | ZNF644 | intron_04 | chr1: 90982371-91020447 | chr1: 91013511-91013530 | CCAAACCAAAUUCCCUAAAC | + |
| 1697 | 84146 | ZNF644 | intron_04 | chr1: 90982371-91020447 | chr1: 91013519-91013538 | UGAAUCCAGUUUAGGGAAUU | − |
| 1698 | 84146 | ZNF644 | intron_04 | chr1: 90982371-91020447 | chr1: 91013514-91013533 | CCAGUUUAGGGAAUUUGGUU | − |
| 1699 | 84146 | ZNF644 | intron_04 | chr1: 90982371-91020447 | chr1: 91013513-91013532 | CAGUUUAGGGAAUUUGGUUU | − |
| 1700 | 84146 | ZNF644 | intron_04 | chr1: 90982371-91020447 | chr1: 91012984-91013003 | AAGAUCGACUAGGCUUUGUU | + |
| 1701 | 84146 | ZNF644 | intron_04 | chr1: 90982371-91020447 | chr1: 91012961-91012980 | AAGGGAAAGUGUUUCAUUUA | − |
| 1702 | 84146 | ZNF644 | intron_04 | chr1: 90982371-91020447 | chr1: 91012912-91012931 | AUGAUGGAGAUUGAUAAAGA | − |
| 1703 | 84146 | ZNF644 | intron_04 | chr1: 90982371-91020447 | chr1: 91012734-91012753 | GGGAGGUACUGGGUUACACC | − |
| 1704 | 84146 | ZNF644 | intron_04 | chr1: 90982371-91020447 | chr1: 91012526-91012545 | UUUAUGGUGAUAGAGGUUAG | + |
| 1705 | 84146 | ZNF644 | intron_04 | chr1: 90982371-91020447 | chr1: 91012225-91012244 | CAUAUUUGUCCUCCCUCCCA | − |
| 1706 | 84146 | ZNF644 | intron_04 | chr1: 90982371-91020447 | chr1: 91012210-91012229 | AAUUCUCACUACCCUUGGGA | + |
| 1707 | 84146 | ZNF644 | intron_04 | chr1: 90982371-91020447 | chr1: 91012224-91012243 | AUAUUUGUCCUCCCUCCCAA | − |
| 1708 | 84146 | ZNF644 | intron_04 | chr1: 90982371-91020447 | chr1: 91012206-91012225 | CAUCAAUUCUCACUACCCUU | + |
| 1709 | 84146 | ZNF644 | intron_04 | chr1: 90982371-91020447 | chr1: 91012205-91012224 | GCAUCAAUUCUCACUACCCU | + |
| 1710 | 84146 | ZNF644 | intron_04 | chr1: 90982371-91020447 | chr1: 91012152-91012171 | UGGCAUUGUUAUGAAUUGGG | − |
| 1711 | 84146 | ZNF644 | intron_04 | chr1: 90982371-91020447 | chr1: 91012143-91012162 | UAUGAAUUGGGAGGUAAAUC | − |
| 1712 | 84146 | ZNF644 | intron_04 | chr1: 90982371-91020447 | chr1: 91012090-91012109 | UCUCUGAUUUUGUCACUGGG | + |
| 1713 | 84146 | ZNF644 | intron_04 | chr1: 90982371-91020447 | chr1: 91012088-91012107 | CCUCUCUGAUUUUGUCACUG | + |
| 1714 | 84146 | ZNF644 | intron_04 | chr1: 90982371-91020447 | chr1: 91012086-91012105 | CACCUCUCUGAUUUUGUCAC | + |
| 1715 | 84146 | ZNF644 | intron_04 | chr1: 90982371-91020447 | chr1: 91012091-91012110 | CCCCAGUGACAAAAUCAGAG | − |
| 1716 | 84146 | ZNF644 | intron_04 | chr1: 90982371-91020447 | chr1: 91011888-91011907 | CAAAAACCAUCUAUAUAAGG | + |
| 1717 | 84146 | ZNF644 | intron_04 | chr1: 90982371-91020447 | chr1: 91011890-91011909 | CUCCUUAUAUAGAUGGUUUU | − |
| 1718 | 84146 | ZNF644 | intron_04 | chr1: 90982371-91020447 | chr1: 91011862-91011881 | CCUGGAAGCUACCAUCAAAG | + |
| 1719 | 84146 | ZNF644 | intron_04 | chr1: 90982371-91020447 | chr1: 91011719-91011738 | AUUCUACUUUUCUGUUGAAC | + |
| 1720 | 84146 | ZNF644 | intron_04 | chr1: 90982371-91020447 | chr1: 91011643-91011662 | AUUUAUAAAGUCUCCUACAU | + |
| 1721 | 84146 | ZNF644 | intron_04 | chr1: 90982371-91020447 | chr1: 91011642-91011661 | CAUUUAUAAAGUCUCCUACA | + |
| 1722 | 84146 | ZNF644 | intron_04 | chr1: 90982371-91020447 | chr1: 91011556-91011575 | UACAACUAUUAAUUCCAUUU | + |
| 1723 | 84146 | ZNF644 | intron_04 | chr1: 90982371-91020447 | chr1: 91011555-91011574 | CUACAACUAUUAAUUCCAUU | + |
| 1724 | 84146 | ZNF644 | intron_04 | chr1: 90982371-91020447 | chr1: 91011500-91011519 | AAUACUGCUAUACUACAAUA | + |
| 1725 | 84146 | ZNF644 | intron_04 | chr1: 90982371-91020447 | chr1: 91011401-91011420 | UCUUCACAUUCUAGUAUAUC | − |
| 1726 | 84146 | ZNF644 | intron_04 | chr1: 90982371-91020447 | chr1: 91011151-91011170 | UAGCUUUUGUGAAGUAUAGA | − |
| 1727 | 84146 | ZNF644 | intron_04 | chr1: 90982371-91020447 | chr1: 91011137-91011156 | UAUAGAAGGCUAAAGCAAAU | − |
| 1728 | 84146 | ZNF644 | intron_04 | chr1: 90982371-91020447 | chr1: 91011063-91011082 | ACUUUACACAUAACAGCUUU | − |
| 1729 | 84146 | ZNF644 | intron_04 | chr1: 90982371-91020447 | chr1: 91011061-91011080 | UUUACACAUAACAGCUUUUG | − |
| 1730 | 84146 | ZNF644 | intron_04 | chr1: 90982371-91020447 | chr1: 91010766-91010785 | AUAUGCAACCCUACCCCUAG | + |
| 1731 | 84146 | ZNF644 | intron_04 | chr1: 90982371-91020447 | chr1: 91010583-91010602 | ACACCACUUCUUCAAAAUCU | + |
| 1732 | 84146 | ZNF644 | intron_04 | chr1: 90982371-91020447 | chr1: 91010121-91010140 | UGGAGGGAUCUAAAGAAGUU | − |
| 1733 | 84146 | ZNF644 | intron_04 | chr1: 90982371-91020447 | chr1: 91009978-91009997 | GUGAUUAUUAUCUUUGUGAG | − |
| 1734 | 84146 | ZNF644 | intron_04 | chr1: 90982371-91020447 | chr1: 91009972-91009991 | AUUAUCUUUGUGAGUGGGAA | − |
| 1735 | 84146 | ZNF644 | intron_04 | chr1: 90982371-91020447 | chr1: 91009722-91009741 | CCAAUAAAGUUAUGCUAUAA | − |
| 1736 | 84146 | ZNF644 | intron_04 | chr1: 90982371-91020447 | chr1: 91009687-91009706 | UAAAAAAUAUUUAAGGUAAC | − |
| 1737 | 84146 | ZNF644 | intron_04 | chr1: 90982371-91020447 | chr1: 91009590-91009609 | UAAUUUGUAUUGGAGCUGGC | − |
| 1738 | 84146 | ZNF644 | intron_04 | chr1: 90982371-91020447 | chr1: 91009123-91009142 | UACCUGUUGGAAAGGAGCGA | + |
| 1739 | 84146 | ZNF644 | intron_04 | chr1: 90982371-91020447 | chr1: 91008908-91008927 | AUGAAGUCCUUUCCAGCUUU | − |
| 1740 | 84146 | ZNF644 | intron_04 | chr1: 90982371-91020447 | chr1: 91008824-91008843 | AGGUGAUACAUGAAUAACCA | + |
| 1741 | 84146 | ZNF644 | intron_04 | chr1: 90982371-91020447 | chr1: 91008764-91008783 | GACGUUAAUAGUGAGGUUUU | − |
| 1742 | 84146 | ZNF644 | intron_04 | chr1: 90982371-91020447 | chr1: 91008746-91008765 | UUUGGGAAUGUGGUAAAACC | − |
| 1743 | 84146 | ZNF644 | intron_04 | chr1: 90982371-91020447 | chr1: 91008725-91008744 | ACAAAGUCAAAUAUACCUCC | + |
| 1744 | 84146 | ZNF644 | intron_04 | chr1: 90982371-91020447 | chr1: 91008682-91008701 | CAGCUAAAGCAAAAUACAGU | − |
| 1745 | 84146 | ZNF644 | intron_04 | chr1: 90982371-91020447 | chr1: 91008650-91008669 | CUCUAGGUCCCAGUGAUAGU | + |
| 1746 | 84146 | ZNF644 | intron_04 | chr1: 90982371-91020447 | chr1: 91008634-91008653 | GAGAUGUAGAUGAAAUCUCU | + |
| 1747 | 84146 | ZNF644 | intron_04 | chr1: 90982371-91020447 | chr1: 91008606-91008625 | GGGGUUUCUGUAUGAUAGUA | − |
| 1748 | 84146 | ZNF644 | intron_04 | chr1: 90982371-91020447 | chr1: 91008597-91008616 | GUAUGAUAGUAUGGGUGAGA | − |
| 1749 | 84146 | ZNF644 | intron_04 | chr1: 90982371-91020447 | chr1: 91008445-91008464 | AGGGCUAUUCCCAAGAACUC | + |
| 1750 | 84146 | ZNF644 | intron_04 | chr1: 90982371-91020447 | chr1: 91008426-91008445 | UCUACAAACAAAUGGAGGUA | + |
| 1751 | 84146 | ZNF644 | intron_04 | chr1: 90982371-91020447 | chr1: 91008425-91008444 | AUCUACAAACAAAUGGAGGU | + |
| 1752 | 84146 | ZNF644 | intron_04 | chr1: 90982371-91020447 | chr1: 91008421-91008440 | UCUCAUCUACAAACAAAUGG | + |
| 1753 | 84146 | ZNF644 | intron_04 | chr1: 90982371-91020447 | chr1: 91008388-91008407 | CCUUCUCCACUGACAGCAGA | + |
| 1754 | 84146 | ZNF644 | intron_04 | chr1: 90982371-91020447 | chr1: 91008332-91008351 | ACCAAUUCCUGAAGCAUUCC | + |
| 1755 | 84146 | ZNF644 | intron_04 | chr1: 90982371-91020447 | chr1: 91008298-91008317 | CUUUUAGACAAUUCCUUCAG | + |
| 1756 | 84146 | ZNF644 | intron_04 | chr1: 90982371-91020447 | chr1: 91008071-91008090 | CCAGUAUUCUCAGAACUGGG | + |
| 1757 | 84146 | ZNF644 | intron_04 | chr1: 90982371-91020447 | chr1: 91008033-91008052 | UUUUAAUCUCACAGCUAGAU | + |
| 1758 | 84146 | ZNF644 | intron_04 | chr1: 90982371-91020447 | chr1: 91008032-91008051 | CUUUUAAUCUCACAGCUAGA | + |
| 1759 | 84146 | ZNF644 | intron_04 | chr1: 90982371-91020447 | chr1: 91008003-91008022 | UCUCAGCAGUUUGCUGUCAC | + |
| 1760 | 84146 | ZNF644 | intron_04 | chr1: 90982371-91020447 | chr1: 91007933-91007952 | UUAAUGAGUAGACAUACUGU | + |
| 1761 | 84146 | ZNF644 | intron_04 | chr1: 90982371-91020447 | chr1: 91007902-91007921 | AGGCACUUAAAAGUUGCCUA | + |
| 1762 | 84146 | ZNF644 | intron_04 | chr1: 90982371-91020447 | chr1: 91007901-91007920 | GAGGCACUUAAAAGUUGCCU | + |
| 1763 | 84146 | ZNF644 | intron_04 | chr1: 90982371-91020447 | chr1: 91007852-91007871 | UAAGGUUAUCCUCAAAUGUU | + |
| 1764 | 84146 | ZNF644 | intron_04 | chr1: 90982371-91020447 | chr1: 91007334-91007353 | CAAGCGAUUUGCUGGGAUUA | + |
| 1765 | 84146 | ZNF644 | intron_04 | chr1: 90982371-91020447 | chr1: 91006250-91006269 | CAGGACCUAGCCAACCACGG | + |
| 1766 | 84146 | ZNF644 | intron_04 | chr1: 90982371-91020447 | chr1: 91005626-91005645 | GAAAUUGGUAAGUUAAUAUG | + |
| 1767 | 84146 | ZNF644 | intron_04 | chr1: 90982371-91020447 | chr1: 91005499-91005518 | AAUGUUCUUUAGUAUGUACU | + |
| 1768 | 84146 | ZNF644 | intron_04 | chr1: 90982371-91020447 | chr1: 91005413-91005432 | AUAGUAUAGAUAGAAGAUCA | + |
| 1769 | 84146 | ZNF644 | intron_04 | chr1: 90982371-91020447 | chr1: 91005029-91005048 | ACAAAAAGCACAUAGGCAGA | + |
| 1770 | 84146 | ZNF644 | intron_04 | chr1: 90982371-91020447 | chr1: 91004970-91004989 | AGAUGUAAAUUCCACUUUAU | + |
| 1771 | 84146 | ZNF644 | intron_04 | chr1: 90982371-91020447 | chr1: 91004572-91004591 | CAUGGUGAAGGUAACCACAU | + |
| 1772 | 84146 | ZNF644 | intron_04 | chr1: 90982371-91020447 | chr1: 91004443-91004462 | CAACUAUUUUUCAUAACAGA | + |
| 1773 | 84146 | ZNF644 | intron_04 | chr1: 90982371-91020447 | chr1: 91004198-91004217 | UAAACAUCCAAAAAAGCUCA | + |
| 1774 | 84146 | ZNF644 | intron_04 | chr1: 90982371-91020447 | chr1: 91004099-91004118 | AUAGGAGUUCUACAAAAGAG | + |
| 1775 | 84146 | ZNF644 | intron_04 | chr1: 90982371-91020447 | chr1: 91003747-91003766 | AGUUCACAAAACUGUCCCUG | + |
| 1776 | 84146 | ZNF644 | intron_04 | chr1: 90982371-91020447 | chr1: 90997780-90997799 | UUAAUUCCCUUUCCAAGCAA | + |
| 1777 | 84146 | ZNF644 | intron_04 | chr1: 90982371-91020447 | chr1: 90997455-90997474 | UAAAAUAACAAACAACGCUG | + |
| 1778 | 84146 | ZNF644 | intron_04 | chr1: 90982371-91020447 | chr1: 90997144-90997163 | GAGAGCCACAUGUACAGAAA | + |
| 1779 | 84146 | ZNF644 | intron_04 | chr1: 90982371-91020447 | chr1: 90997087-90997106 | GCUUUAAAAAGAAAAGCUGG | + |
| 1780 | 84146 | ZNF644 | intron_04 | chr1: 90982371-91020447 | chr1: 90996883-90996902 | UAACACAUCUGGUGGUCUCC | + |
| 1781 | 84146 | ZNF644 | intron_04 | chr1: 90982371-91020447 | chr1: 90996872-90996891 | CUGUCAUUAUUUAACACAUC | + |
| 1782 | 84146 | ZNF644 | intron_04 | chr1: 90982371-91020447 | chr1: 90996573-90996592 | CCAAGUGCUUGGAACCAGUC | + |
| 1783 | 84146 | ZNF644 | intron_04 | chr1: 90982371-91020447 | chr1: 90996562-90996581 | AAUGCUUGAGUCCAAGUGCU | + |
| 1784 | 84146 | ZNF644 | intron_04 | chr1: 90982371-91020447 | chr1: 90996418-90996437 | GGGAAGACCAGCACACUUUA | + |
| 1785 | 84146 | ZNF644 | intron_04 | chr1: 90982371-91020447 | chr1: 90996398-90996417 | UAGAUGGCUGAAUCUUAUUU | + |
| 1786 | 84146 | ZNF644 | intron_04 | chr1: 90982371-91020447 | chr1: 90996397-90996416 | AUAGAUGGCUGAAUCUUAUU | + |
| 1787 | 84146 | ZNF644 | intron_04 | chr1: 90982371-91020447 | chr1: 90996301-90996320 | CAUAAACCCAGCAAGAUCAC | + |
| 1788 | 84146 | ZNF644 | intron_04 | chr1: 90982371-91020447 | chr1: 90996091-90996110 | AAAAGGUACUCAGUCACAUU | + |
| 1789 | 84146 | ZNF644 | intron_04 | chr1: 90982371-91020447 | chr1: 90996074-90996093 | CUGGCAAUACAUAUAUUAAA | + |
| 1790 | 84146 | ZNF644 | intron_04 | chr1: 90982371-91020447 | chr1: 90995855-90995874 | AAUCAAAAACAUGACAUCAG | + |
| 1791 | 84146 | ZNF644 | intron_04 | chr1: 90982371-91020447 | chr1: 90995377-90995396 | ACAUAUCCUAAGAGACCAGU | + |
| 1792 | 84146 | ZNF644 | intron_04 | chr1: 90982371-91020447 | chr1: 90994924-90994943 | CAAGUUUGCAGUCUGGGCCC | + |
| 1793 | 84146 | ZNF644 | intron_04 | chr1: 90982371-91020447 | chr1: 90994918-90994937 | GGAGACCAAGUUUGCAGUCU | + |
| 1794 | 84146 | ZNF644 | intron_04 | chr1: 90982371-91020447 | chr1: 90994814-90994833 | AACCAACUUACUCAGACACU | + |
| 1795 | 84146 | ZNF644 | intron_04 | chr1: 90982371-91020447 | chr1: 90994715-90994734 | UAGAAGCUCUGUAGAACAGC | + |
| 1796 | 84146 | ZNF644 | intron_04 | chr1: 90982371-91020447 | chr1: 90994647-90994666 | AUUCCCCAACUAUGCAGUGU | + |
| 1797 | 84146 | ZNF644 | intron_04 | chr1: 90982371-91020447 | chr1: 90994539-90994558 | GAGAAGACCUGAAGGGAUUG | + |
| 1798 | 84146 | ZNF644 | intron_04 | chr1: 90982371-91020447 | chr1: 90994531-90994550 | UAGGCACAGAGAAGACCUGA | + |
| 1799 | 84146 | ZNF644 | intron_04 | chr1: 90982371-91020447 | chr1: 90994512-90994531 | UGGAUGACUGAUAAACCAGU | + |
| 1800 | 84146 | ZNF644 | intron_04 | chr1: 90982371-91020447 | chr1: 90994449-90994468 | AUGGCAGUCAGAGAACCAGA | + |
| 1801 | 84146 | ZNF644 | intron_04 | chr1: 90982371-91020447 | chr1: 90994448-90994467 | CAUGGCAGUCAGAGAACCAG | + |
| 1802 | 84146 | ZNF644 | intron_04 | chr1: 90982371-91020447 | chr1: 90994420-90994439 | AAACAGCUAGAAAUUUAAAU | + |
| 1803 | 84146 | ZNF644 | intron_04 | chr1: 90982371-91020447 | chr1: 90994377-90994396 | AUAAAGACAGUAUCACUGUA | + |
| 1804 | 84146 | ZNF644 | intron_04 | chr1: 90982371-91020447 | chr1: 90994376-90994395 | GAUAAAGACAGUAUCACUGU | + |
| 1805 | 84146 | ZNF644 | intron_04 | chr1: 90982371-91020447 | chr1: 90994354-90994373 | AAGCACACAAAAAUUAUCUC | + |
| 1806 | 84146 | ZNF644 | intron_04 | chr1: 90982371-91020447 | chr1: 90994353-90994372 | AGAUAAUUUUUGUGUGCUUG | − |
| 1807 | 84146 | ZNF644 | intron_04 | chr1: 90982371-91020447 | chr1: 90994203-90994222 | UUCAUGGAGAGAACAUGUUC | + |
| 1808 | 84146 | ZNF644 | intron_04 | chr1: 90982371-91020447 | chr1: 90994100-90994119 | GUACAUUUGCAAAAACUAUU | + |
| 1809 | 84146 | ZNF644 | intron_04 | chr1: 90982371-91020447 | chr1: 90994099-90994118 | AGUACAUUUGCAAAAACUAU | + |
| 1810 | 84146 | ZNF644 | intron_04 | chr1: 90982371-91020447 | chr1: 90993998-90994017 | AGGAUAAACAAGUGACUCUC | + |
| 1811 | 84146 | ZNF644 | intron_04 | chr1: 90982371-91020447 | chr1: 90993963-90993982 | AUUACAGUGGCCCCAAUCCU | + |
| 1812 | 84146 | ZNF644 | intron_04 | chr1: 90982371-91020447 | chr1: 90993950-90993969 | UUCUUACUCAACUAUUACAG | + |
| 1813 | 84146 | ZNF644 | intron_04 | chr1: 90982371-91020447 | chr1: 90993938-90993957 | AGUAAGAAUGUGUCCCUAUC | 1 |
| 1814 | 84146 | ZNF644 | intron_04 | chr1: 90982371-91020447 | chr1: 90993922-90993941 | GUAACCUUAAUUUCCAGAUA | + |
| 1815 | 84146 | ZNF644 | intron_04 | chr1: 90982371-91020447 | chr1: 90993747-90993766 | CAUUACUUACAAAAUGAUUU | − |
| 1816 | 84146 | ZNF644 | intron_04 | chr1: 90982371-91020447 | chr1: 90993627-90993646 | UUAACUUUUGAAGACAAGGG | + |
| 1817 | 84146 | ZNF644 | intron_04 | chr1: 90982371-91020447 | chr1: 90993625-90993644 | UCUUAACUUUUGAAGACAAG | + |
| 1818 | 84146 | ZNF644 | intron_04 | chr1: 90982371-91020447 | chr1: 90993623-90993642 | UCUCUUAACUUUUGAAGACA | + |
| 1819 | 84146 | ZNF644 | intron_04 | chr1: 90982371-91020447 | chr1: 90993571-90993590 | AAUAAGUUUUUCUUAGCCCU | + |
| 1820 | 84146 | ZNF644 | intron_04 | chr1: 90982371-91020447 | chr1: 90993312-90993331 | GCCAUCCUAGAUCAACCUUA | + |
| 1821 | 84146 | ZNF644 | intron_04 | chr1: 90982371-91020447 | chr1: 90993235-90993254 | CUCAUCUGCUUCAGAAUGGG | + |
| 1822 | 84146 | ZNF644 | intron_04 | chr1: 90982371-91020447 | chr1: 90993078-90993097 | CUCCCUGAUCUCUUAAAUCU | + |
| 1823 | 84146 | ZNF644 | intron_04 | chr1: 90982371-91020447 | chr1: 90992582-90992601 | UUGCAUAUAGAAAUUGGGGC | − |
| 1824 | 84146 | ZNF644 | intron_04 | chr1: 90982371-91020447 | chr1: 90992496-90992515 | CAUCAGGUCUGAUGAGAUUU | − |
| 1825 | 84146 | ZNF644 | intron_04 | chr1: 90982371-91020447 | chr1: 90992461-90992480 | UAGACUGAAUUACCAGUUGU | − |
| 1826 | 84146 | ZNF644 | intron_04 | chr1: 90982371-91020447 | chr1: 90992379-90992398 | UGGUUAUAAAUGAUUUUUUU | − |
| 1827 | 84146 | ZNF644 | intron_04 | chr1: 90982371-91020447 | chr1: 90992200-90992219 | CUUAUCUUGAAUGAUUUCAU | + |
| 1828 | 84146 | ZNF644 | intron_04 | chr1: 90982371-91020447 | chr1: 90992149-90992168 | GAAGAAUACCAACAGGCAUA | + |
| 1829 | 84146 | ZNF644 | intron_04 | chr1: 90982371-91020447 | chr1: 90992142-90992161 | CAAACAGGAAGAAUACCAAC | + |
| 1830 | 84146 | ZNF644 | intron_04 | chr1: 90982371-91020447 | chr1: 90992127-90992146 | CAUUCACUGUCUGGGCAAAC | + |
| 1831 | 84146 | ZNF644 | intron_04 | chr1: 90982371-91020447 | chr1: 90992119-90992138 | AAGCAGAUCAUUCACUGUCU | + |
| 1832 | 84146 | ZNF644 | intron_04 | chr1: 90982371-91020447 | chr1: 90991393-90991412 | CCUACUACAGGAUCCAGUCA | + |
| 1833 | 84146 | ZNF644 | intron_04 | chr1: 90982371-91020447 | chr1: 90991123-90991142 | GAAAAUUUGAAGCAGCACUU | + |
| 1834 | 84146 | ZNF644 | intron_04 | chr1: 90982371-91020447 | chr1: 90990917-90990936 | AAGAACUACAAAGUGGCACC | + |
| 1835 | 84146 | ZNF644 | intron_04 | chr1: 90982371-91020447 | chr1: 90990892-90990911 | ACCCCCAAAAAGGAACGUGG | + |
| 1836 | 84146 | ZNF644 | intron_04 | chr1: 90982371-91020447 | chr1: 90990720-90990739 | AGUCACCCAAAAGGAUGAAA | + |
| 1837 | 84146 | ZNF644 | intron_04 | chr1: 90982371-91020447 | chr1: 90990694-90990713 | GUGUGGAAACUAUCCAAGCA | + |
| 1838 | 84146 | ZNF644 | intron_04 | chr1: 90982371-91020447 | chr1: 90990562-90990581 | UGGUGGUAAUCCUUGAGUUG | + |
| 1839 | 84146 | ZNF644 | intron_04 | chr1: 90982371-91020447 | chr1: 90990545-90990564 | AGUAUCUAUGCAGAAUCUGG | + |
| 1840 | 84146 | ZNF644 | intron_04 | chr1: 90982371-91020447 | chr1: 90990542-90990561 | AAGAGUAUCUAUGCAGAAUC | + |
| 1841 | 84146 | ZNF644 | intron_04 | chr1: 90982371-91020447 | chr1: 90990449-90990468 | AGACAUUAGGCAACCCCUGA | + |
| 1842 | 84146 | ZNF644 | intron_04 | chr1: 90982371-91020447 | chr1: 90990355-90990374 | AAGAGCCAAGGUGGAGUAAC | + |
| 1843 | 84146 | ZNF644 | intron_04 | chr1: 90982371-91020447 | chr1: 90990131-90990150 | UGGAGUGGUUGUCAAAAUCU | + |
| 1844 | 84146 | ZNF644 | intron_04 | chr1: 90982371-91020447 | chr1: 90990130-90990149 | GUGGAGUGGUUGUCAAAAUC | + |
| 1845 | 84146 | ZNF644 | intron_04 | chr1: 90982371-91020447 | chr1: 90990019-90990038 | AUGAACCUCAAUCACCUCAA | + |
| 1846 | 84146 | ZNF644 | intron_04 | chr1: 90982371-91020447 | chr1: 90990018-90990037 | GAUGAACCUCAAUCACCUCA | + |
| 1847 | 84146 | ZNF644 | intron_04 | chr1: 90982371-91020447 | chr1: 90989696-90989715 | GGAACCCUUGGUGCACUGUU | + |
| 1848 | 84146 | ZNF644 | intron_04 | chr1: 90982371-91020447 | chr1: 90989196-90989215 | AAAUGGGCCAAGUACUUCAA | + |
| 1849 | 84146 | ZNF644 | intron_04 | chr1: 90982371-91020447 | chr1: 90988937-90988956 | GAAAACAUAGGGGAAGGCUU | + |
| 1850 | 84146 | ZNF644 | intron_04 | chr1: 90982371-91020447 | chr1: 90988769-90988788 | CAAUACUAUACAGAGAGGAA | + |
| 1851 | 84146 | ZNF644 | intron_04 | chr1: 90982371-91020447 | chr1: 90988730-90988749 | CCCAUGCAUUUCUAGUCAAA | + |
| 1852 | 84146 | ZNF644 | intron_04 | chr1: 90982371-91020447 | chr1: 90987504-90987523 | UAUAACUAGUAAACAGAGAU | + |
| 1853 | 84146 | ZNF644 | intron_04 | chr1: 90982371-91020447 | chr1: 90987453-90987472 | AAUGUACACCUAGCAAAACG | + |
| 1854 | 84146 | ZNF644 | intron_04 | chr1: 90982371-91020447 | chr1: 90987192-90987211 | UAAAUAUAGAACUGAAGACA | + |
| 1855 | 84146 | ZNF644 | intron_04 | chr1: 90982371-91020447 | chr1: 90987136-90987155 | UAAACCAAAUAGUAGCAGAA | + |
| 1856 | 84146 | ZNF644 | intron_04 | chr1: 90982371-91020447 | chr1: 90986910-90986929 | CUUACUCUUACCCAACCAAU | + |
| 1857 | 84146 | ZNF644 | intron_04 | chr1: 90982371-91020447 | chr1: 90986560-90986579 | GGAAGGGGUGGUUAGAAUAU | + |
| 1858 | 84146 | ZNF644 | intron_04 | chr1: 90982371-91020447 | chr1: 90986559-90986578 | UGGAAGGGGUGGUUAGAAUA | + |
| 1859 | 84146 | ZNF644 | intron_04 | chr1: 90982371-91020447 | chr1: 90986545-90986564 | GGAAAAAUGACAGUUGGAAG | + |
| 1860 | 84146 | ZNF644 | intron_04 | chr1: 90982371-91020447 | chr1: 90986539-90986558 | CAACAGGGAAAAAUGACAGU | + |
| 1861 | 84146 | ZNF644 | intron_04 | chr1: 90982371-91020447 | chr1: 90986358-90986377 | AACAAAGUCUUCAGAAACUU | + |
| 1862 | 84146 | ZNF644 | intron_04 | chr1: 90982371-91020447 | chr1: 90985908-90985927 | AUUCCUAACAAGGUACACAA | + |
| 1863 | 84146 | ZNF644 | intron_04 | chr1: 90982371-91020447 | chr1: 90985898-90985917 | UUUAUUUUGCAUUCCUAACA | + |
| 1864 | 84146 | ZNF644 | intron_04 | chr1: 90982371-91020447 | chr1: 90985533-90985552 | UACAUGUAAGUUAGAUUACG | + |
| 1865 | 84146 | ZNF644 | intron_04 | chr1: 90982371-91020447 | chr1: 90985136-90985155 | UGUCUAAAAUGUGCUGGUUU | − |
| 1866 | 84146 | ZNF644 | intron_04 | chr1: 90982371-91020447 | chr1: 90985129-90985148 | AAUGUGCUGGUUUAGGUUUG | − |
| 1867 | 84146 | ZNF644 | intron_04 | chr1: 90982371-91020447 | chr1: 90985128-90985147 | AUGUGCUGGUUUAGGUUUGU | − |
| 1868 | 84146 | ZNF644 | intron_04 | chr1: 90982371-91020447 | chr1: 90985127-90985146 | UGUGCUGGUUUAGGUUUGUG | − |
| 1869 | 84146 | ZNF644 | intron_04 | chr1: 90982371-91020447 | chr1: 90985108-90985127 | GGGGAACAAAUGCAGUUAAA | − |
| 1870 | 84146 | ZNF644 | intron_04 | chr1: 90982371-91020447 | chr1: 90984716-90984735 | ACAAGGUAUGCACACACAGA | + |
| 1871 | 84146 | ZNF644 | intron_04 | chr1: 90982371-91020447 | chr1: 90984242-90984261 | AAGUUCUCAAGGAGAUAAAC | + |
| 1872 | 84146 | ZNF644 | intron_04 | chr1: 90982371-91020447 | chr1: 90984027-90984046 | ACCAGUAUCAGGGAGAAGCA | + |
| 1873 | 84146 | ZNF644 | intron_04 | chr1: 90982371-91020447 | chr1: 90984017-90984036 | GCCAGCACCCACCAGUAUCA | + |
| 1874 | 84146 | ZNF644 | intron_04 | chr1: 90982371-91020447 | chr1: 90983313-90983332 | CACCAACCCAGAAGUGGACA | + |
| 1875 | 84146 | ZNF644 | intron_04 | chr1: 90982371-91020447 | chr1: 90983322-90983341 | AGGCCCCCAUGUCCACUUCU | − |
| 1876 | 84146 | ZNF644 | intron_04 | chr1: 90982371-91020447 | chr1: 90983318-90983337 | CCCCAUGUCCACUUCUGGGU | − |
| 1877 | 84146 | ZNF644 | intron_04 | chr1: 90982371-91020447 | chr1: 90983315-90983334 | CAUGUCCACUUCUGGGUUGG | − |
| 1878 | 84146 | ZNF644 | intron_04 | chr1: 90982371-91020447 | chr1: 90983155-90983174 | GAGAACAGAGGUAGGUGUGG | − |
| 1879 | 84146 | ZNF644 | intron_04 | chr1: 90982371-91020447 | chr1: 90983103-90983122 | AAGAGUCACAAGCUAGUUUC | − |
| 1880 | 84146 | ZNF644 | intron_04 | chr1: 90982371-91020447 | chr1: 90983093-90983112 | AGCUAGUUUCAGGUAUGGGG | − |
| 1881 | 84146 | ZNF644 | intron_04 | chr1: 90982371-91020447 | chr1: 90983072-90983091 | GGAGGAGAUAUGUAGAGUGU | − |
| 1882 | 84146 | ZNF644 | intron_04 | chr1: 90982371-91020447 | chr1: 90982983-90983002 | AUGCAUGUUCAGUGGAUUGU | − |
| 1883 | 84146 | ZNF644 | intron_04 | chr1: 90982371-91020447 | chr1: 90982851-90982870 | GGCUAUUAUGAUAAUUACUG | − |
| 1884 | 84146 | ZNF644 | intron_04 | chr1: 90982371-91020447 | chr1: 90982648-90982667 | UUUCAUUAAUUUUAAUAGUA | + |
| 1885 | 84146 | ZNF644 | intron_04 | chr1: 90982371-91020447 | chr1: 90982522-90982541 | UUUCCUAAAGAUUAAAAUGA | − |
| 1886 | 84146 | ZNF644 | intron_04 | chr1: 90982371-91020447 | chr1: 90982506-90982525 | AUGAUGGUAUUCUGGUUGAA | − |
| 1887 | 84146 | ZNF644 | intron_04 | chr1: 90982371-91020447 | chr1: 90982376-90982395 | AGAAAUACACACAAUGACCA | + |
| 1888 | 84146 | ZNF644 | intron_05 | chr1: 90973214-90982309 | chr1: 90982270-90982289 | UUAAAUGUACAUAUCAAGGU | − |
| 1889 | 84146 | ZNF644 | intron_05 | chr1: 90973214-90982309 | chr1: 90982136-90982155 | UUUACUCCCAUCAAAUCAAC | + |
| 1890 | 84146 | ZNF644 | intron_05 | chr1: 90973214-90982309 | chr1: 90982118-90982137 | AAAUUAUAACUAUCACAUUG | − |
| 1891 | 84146 | ZNF644 | intron_05 | chr1: 90973214-90982309 | chr1: 90982036-90982055 | UAACAUUUUAGAAUAUUCAC | + |
| 1892 | 84146 | ZNF644 | intron_05 | chr1: 90973214-90982309 | chr1: 90981865-90981884 | AAGGAAGCACUUUCCCAAAU | − |
| 1893 | 84146 | ZNF644 | intron_05 | chr1: 90973214-90982309 | chr1: 90981741-90981760 | AUCAGAAUGCUAUAUUAGAG | + |
| 1894 | 84146 | ZNF644 | intron_05 | chr1: 90973214-90982309 | chr1: 90981604-90981623 | UAUUUAUUGGUAAGUGAAUA | − |
| 1895 | 84146 | ZNF644 | intron_05 | chr1: 90973214-90982309 | chr1: 90981584-90981603 | UGGCUUCAAAAUGGAAACAA | − |
| 1896 | 84146 | ZNF644 | intron_05 | chr1: 90973214-90982309 | chr1: 90981512-90981531 | UUUUUGACAGCUGAAACAUC | + |
| 1897 | 84146 | ZNF644 | intron_05 | chr1: 90973214-90982309 | chr1: 90981436-90981455 | AGUUCAGUAAUAUGCACUGA | − |
| 1898 | 84146 | ZNF644 | intron_05 | chr1: 90973214-90982309 | chr1: 90981420-90981439 | CUGAAGGAAAUCUCAGUCAG | − |
| 1899 | 84146 | ZNF644 | intron_05 | chr1: 90973214-90982309 | chr1: 90981415-90981434 | GGAAAUCUCAGUCAGUGGUU | − |
| 1900 | 84146 | ZNF644 | intron_05 | chr1: 90973214-90982309 | chr1: 90981260-90981279 | UUUUUCUACACAACAAGAGG | + |
| 1901 | 84146 | ZNF644 | intron_05 | chr1: 90973214-90982309 | chr1: 90981159-90981178 | GCCUAUGUAUCUUCUUAAAG | − |
| 1902 | 84146 | ZNF644 | intron_05 | chr1: 90973214-90982309 | chr1: 90981130-90981149 | AGCCAUUCUUUUGUACUUGA | − |
| 1903 | 84146 | ZNF644 | intron_05 | chr1: 90973214-90982309 | chr1: 90981120-90981139 | UUGUACUUGAAGGUCUUAUU | − |
| 1904 | 84146 | ZNF644 | intron_05 | chr1: 90973214-90982309 | chr1: 90981044-90981063 | CUAUGUUUGAUAUUGCUUGU | − |
| 1905 | 84146 | ZNF644 | intron_05 | chr1: 90973214-90982309 | chr1: 90980816-90980835 | CUGGAAGCGGGGAAAGCACA | + |
| 1906 | 84146 | ZNF644 | intron_05 | chr1: 90973214-90982309 | chr1: 90980778-90980797 | UCAGAAAGAUGAUUACUUCA | + |
| 1907 | 84146 | ZNF644 | intron_05 | chr1: 90973214-90982309 | chr1: 90980516-90980535 | GUAUAAAAAAGUUAGCAAGU | + |
| 1908 | 84146 | ZNF644 | intron_05 | chr1: 90973214-90982309 | chr1: 90980477-90980496 | UGGGGGACAAUUAAAGAGAC | + |
| 1909 | 84146 | ZNF644 | intron_05 | chr1: 90973214-90982309 | chr1: 90980441-90980460 | AUGUAGUAAACCUUAUAACA | − |
| 1910 | 84146 | ZNF644 | intron_05 | chr1: 90973214-90982309 | chr1: 90980400-90980419 | UCAGCAUUCUGGCAUAGCUA | + |
| 1911 | 84146 | ZNF644 | intron_05 | chr1: 90973214-90982309 | chr1: 90980340-90980359 | AUGUACCUAGGACUAGAACU | + |
| 1912 | 84146 | ZNF644 | intron_05 | chr1: 90973214-90982309 | chr1: 90980328-90980347 | GAAAGUGACAACAUGUACCU | + |
| 1913 | 84146 | ZNF644 | intron_05 | chr1: 90973214-90982309 | chr1: 90980280-90980299 | ACAACUAGCAAAGAGGUCUC | + |
| 1914 | 84146 | ZNF644 | intron_05 | chr1: 90973214-90982309 | chr1: 90980273-90980292 | UAAGCUGACAACUAGCAAAG | + |
| 1915 | 84146 | ZNF644 | intron_05 | chr1: 90973214-90982309 | chr1: 90980052-90980071 | UAGAGAUUCAGAAUGCUAGC | − |
| 1916 | 84146 | ZNF644 | intron_05 | chr1: 90973214-90982309 | chr1: 90980046-90980065 | UUCAGAAUGCUAGCUGGAAU | − |
| 1917 | 84146 | ZNF644 | intron_05 | chr1: 90973214-90982309 | chr1: 90980041-90980060 | AAUGCUAGCUGGAAUGGGAU | − |
| 1918 | 84146 | ZNF644 | intron_05 | chr1: 90973214-90982309 | chr1: 90980040-90980059 | AUGCUAGCUGGAAUGGGAUG | − |
| 1919 | 84146 | ZNF644 | intron_05 | chr1: 90973214-90982309 | chr1: 90980039-90980058 | UGCUAGCUGGAAUGGGAUGG | − |
| 1920 | 84146 | ZNF644 | intron_05 | chr1: 90973214-90982309 | chr1: 90980015-90980034 | AUUGGCACUUGAGUUGCUGU | − |
| 1921 | 84146 | ZNF644 | intron_05 | chr1: 90973214-90982309 | chr1: 90979914-90979933 | AACUGGAUUGAAUGAAGACU | + |
| 1922 | 84146 | ZNF644 | intron_05 | chr1: 90973214-90982309 | chr1: 90979709-90979728 | ACUAAUACAAGUAGAAGAUU | − |
| 1923 | 84146 | ZNF644 | intron_05 | chr1: 90973214-90982309 | chr1: 90979558-90979577 | AUAGCAAGCUUCCUAAGCUG | + |
| 1924 | 84146 | ZNF644 | intron_05 | chr1: 90973214-90982309 | chr1: 90979130-90979149 | UUUUAAAGCAUUUUGACUAG | − |
| 1925 | 84146 | ZNF644 | intron_05 | chr1: 90973214-90982309 | chr1: 90979034-90979053 | CUAGAGGGCAAUCAAGCUUC | + |
| 1926 | 84146 | ZNF644 | intron_05 | chr1: 90973214-90982309 | chr1: 90979019-90979038 | CUUGCUUUGGACUUGCUAGA | + |
| 1927 | 84146 | ZNF644 | intron_05 | chr1: 90973214-90982309 | chr1: 90979018-90979037 | UCUUGCUUUGGACUUGCUAG | + |
| 1928 | 84146 | ZNF644 | intron_05 | chr1: 90973214-90982309 | chr1: 90979002-90979021 | AAGAAGAGGUUCUUCCCCAC | − |
| 1929 | 84146 | ZNF644 | intron_05 | chr1: 90973214-90982309 | chr1: 90978984-90979003 | AGCAACAAAUUGAUCCCUGU | + |
| 1930 | 84146 | ZNF644 | intron_05 | chr1: 90973214-90982309 | chr1: 90978983-90979002 | AAGCAACAAAUUGAUCCCUG | + |
| 1931 | 84146 | ZNF644 | intron_05 | chr1: 90973214-90982309 | chr1: 90978941-90978960 | AACUCUGUGUUCAAACAUUA | + |
| 1932 | 84146 | ZNF644 | intron_05 | chr1: 90973214-90982309 | chr1: 90978858-90978877 | UUCUUACCGUAUAUAGACCU | − |
| 1933 | 84146 | ZNF644 | intron_05 | chr1: 90973214-90982309 | chr1: 90978810-90978829 | ACUAAACACACUUCACAAUC | + |
| 1934 | 84146 | ZNF644 | intron_05 | chr1: 90973214-90982309 | chr1: 90978737-90978756 | UUUGUGGAGACAUGUAAGUU | + |
| 1935 | 84146 | ZNF644 | intron_05 | chr1: 90973214-90982309 | chr1: 90978673-90978692 | AGACGAAGACUCUGCAACGG | + |
| 1936 | 84146 | ZNF644 | intron_05 | chr1: 90973214-90982309 | chr1: 90978670-90978689 | UGAAGACGAAGACUCUGCAA | + |
| 1937 | 84146 | ZNF644 | intron_05 | chr1: 90973214-90982309 | chr1: 90978589-90978608 | CAUAAAGAAUUUCAAAUCCU | + |
| 1938 | 84146 | ZNF644 | intron_05 | chr1: 90973214-90982309 | chr1: 90978481-90978500 | UCUUUUUCAUGCUACAACAA | + |
| 1939 | 84146 | ZNF644 | intron_05 | chr1: 90973214-90982309 | chr1: 90977925-90977944 | CAGCUAUUUUUGGUAACUUA | + |
| 1940 | 84146 | ZNF644 | intron_05 | chr1: 90973214-90982309 | chr1: 90977672-90977691 | UUUAUUACAUCAGAAGUUAA | + |
| 1941 | 84146 | ZNF644 | intron_05 | chr1: 90973214-90982309 | chr1: 90977401-90977420 | CACAAAGGUAUGUUAAUUGA | + |
| 1942 | 84146 | ZNF644 | intron_05 | chr1: 90973214-90982309 | chr1: 90977317-90977336 | AUCCUAAAAAAAUAGUAUGA | + |
| 1943 | 84146 | ZNF644 | intron_05 | chr1: 90973214-90982309 | chr1: 90977212-90977231 | AUGAACCUAAUAUUUAGUAA | + |
| 1944 | 84146 | ZNF644 | intron_05 | chr1: 90973214-90982309 | chr1: 90976859-90976878 | AUAGUACUUUAAACAUAUCU | + |
| 1945 | 84146 | ZNF644 | intron_05 | chr1: 90973214-90982309 | chr1: 90976769-90976788 | UUGACCCUUCAUAUCUGAAG | + |
| 1946 | 84146 | ZNF644 | intron_05 | chr1: 90973214-90982309 | chr1: 90976768-90976787 | GUUGACCCUUCAUAUCUGAA | + |
| 1947 | 84146 | ZNF644 | intron_05 | chr1: 90973214-90982309 | chr1: 90976724-90976743 | CAUCUCUUAAAACUCACCCU | − |
| 1948 | 84146 | ZNF644 | intron_05 | chr1: 90973214-90982309 | chr1: 90976705-90976724 | UAACAGCACAGUGAAACCAA | + |
| 1949 | 84146 | ZNF644 | intron_05 | chr1: 90973214-90982309 | chr1: 90976693-90976712 | UGCUGUUAAAUUUGUUUAAA | − |
| 1950 | 84146 | ZNF644 | intron_05 | chr1: 90973214-90982309 | chr1: 90976607-90976626 | UUUUAUACUAAAGACAAGCA | + |
| 1951 | 84146 | ZNF644 | intron_05 | chr1: 90973214-90982309 | chr1: 90976554-90976573 | UUUGCCGCUUAGCCUUUGUU | + |
| 1952 | 84146 | ZNF644 | intron_05 | chr1: 90973214-90982309 | chr1: 90976534-90976553 | UAUCAUUCCAAUAUGUGAAC | − |
| 1953 | 84146 | ZNF644 | intron_05 | chr1: 90973214-90982309 | chr1: 90976393-90976412 | AGAAUAUUAGAUUUCAAUAA | + |
| 1954 | 84146 | ZNF644 | intron_05 | chr1: 90973214-90982309 | chr1: 90976327-90976346 | AUAACUAGUUGCAUGGUACU | + |
| 1955 | 84146 | ZNF644 | intron_05 | chr1: 90973214-90982309 | chr1: 90976320-90976339 | UUGUACUAUAACUAGUUGCA | + |
| 1956 | 84146 | ZNF644 | intron_05 | chr1: 90973214-90982309 | chr1: 90976279-90976298 | UACACUAUACUACUCAGACU | + |
| 1957 | 84146 | ZNF644 | intron_05 | chr1: 90973214-90982309 | chr1: 90976248-90976267 | UCCUUAAAAACGUCCUCCUC | + |
| 1958 | 84146 | ZNF644 | intron_05 | chr1: 90973214-90982309 | chr1: 90976024-90976043 | UUCACAUUUUACCACAGCUU | − |
| 1959 | 84146 | ZNF644 | intron_05 | chr1: 90973214-90982309 | chr1: 90975977-90975996 | CAGUUUGAAAAAAUAGCACA | + |
| 1960 | 84146 | ZNF644 | intron_05 | chr1: 90973214-90982309 | chr1: 90975741-90975760 | GCCUCAAAUAUCUACUUUAA | + |
| 1961 | 84146 | ZNF644 | intron_05 | chr1: 90973214-90982309 | chr1: 90975745-90975764 | ACCAUUAAAGUAGAUAUUUG | − |
| 1962 | 84146 | ZNF644 | intron_05 | chr1: 90973214-90982309 | chr1: 90975417-90975436 | GUAGAUAUUUGAAACUUUUA | − |
| 1963 | 84146 | ZNF644 | intron_05 | chr1: 90973214-90982309 | chr1: 90975371-90975390 | ACAAAAAUUUACUGAGCCCA | + |
| 1964 | 84146 | ZNF644 | intron_05 | chr1: 90973214-90982309 | chr1: 90975335-90975354 | UCAUCUGAUGUUUUAUGAAU | − |
| 1965 | 84146 | ZNF644 | intron_05 | chr1: 90973214-90982309 | chr1: 90974009-90974028 | CUUCUCUCCUAAAACUUUUA | + |
| 1966 | 84146 | ZNF644 | intron_05 | chr1: 90973214-90982309 | chr1: 90974019-90974038 | ACCUAUCCCAUAAAAGUUUU | − |
| 1967 | 84146 | ZNF644 | intron_05 | chr1: 90973214-90982309 | chr1: 90973726-90973745 | UCAUCUAUAGAGGGCACUGC | + |
| 1968 | 84146 | ZNF644 | intron_05 | chr1: 90973214-90982309 | chr1: 90973704-90973723 | GGCUCUUCUAUUUUUUAAAC | − |
| 1969 | 84146 | ZNF644 | intron_05 | chr1: 90973214-90982309 | chr1: 90973306-90973325 | UAACAUAAGAAAUACUACAC | − |
| 1970 | 84146 | ZNF644 | intron_05 | chr1: 90973214-90982309 | chr1: 90973236-90973255 | UGAGAGUAUAAUACAUAAAU | + |
| 1971 | 84146 | ZNF644 | exon_06_nc.1 | chr1: 90973153-90973213 | chr1: 90973174-90973193 | CAAUAAAUAACAAAUAUGUC | + |
| 1972 | 84146 | ZNF644 | exon_06_nc.1 | chr1: 90973153-90973213 | chr1: 90973144-90973163 | AAAAAUUACCAAUGGGGAUG | + |
| 1973 | 84146 | ZNF644 | exon_06_nc.1 | chr1: 90973153-90973213 | chr1: 90973155-90973174 | GAUCUCCACCUCAUCCCCAU | − |
| 1974 | 84146 | ZNF644 | exon_06_nc.2 | chr1: 90972744-90973152 | chr1: 90972940-90972959 | CUCAGGAGCAGGAGAAACGG | + |
| 1975 | 84146 | ZNF644 | exon_06_nc.2 | chr1: 90972744-90973152 | chr1: 90972937-90972956 | GACCUCAGGAGCAGGAGAAA | + |
| 1976 | 84146 | ZNF644 | intron_06 | chr1: 90941310-90972743 | chr1: 90972229-90972248 | GCAAAUCAUGUUAUCUAACA | + |
| 1977 | 84146 | ZNF644 | intron_06 | chr1: 90941310-90972743 | chr1: 90971861-90971880 | GAAAUGAUGAAAAAAGGGCC | − |
| 1978 | 84146 | ZNF644 | intron_06 | chr1: 90941310-90972743 | chr1: 90971840-90971859 | CUUUCAAUCAGAUAGAAACC | + |
| 1979 | 84146 | ZNF644 | intron_06 | chr1: 90941310-90972743 | chr1: 90971758-90971777 | AGCCAGAGCACUUAGGGGAG | + |
| 1980 | 84146 | ZNF644 | intron_06 | chr1: 90941310-90972743 | chr1: 90971757-90971776 | UAGCCAGAGCACUUAGGGGA | + |
| 1981 | 84146 | ZNF644 | intron_06 | chr1: 90941310-90972743 | chr1: 90971756-90971775 | CUAGCCAGAGCACUUAGGGG | + |
| 1982 | 84146 | ZNF644 | intron_06 | chr1: 90941310-90972743 | chr1: 90971753-90971772 | UUCCUAGCCAGAGCACUUAG | + |
| 1983 | 84146 | ZNF644 | intron_06 | chr1: 90941310-90972743 | chr1: 90970573-90970592 | AAUUCCAAUCUAAAUGACUC | + |
| 1984 | 84146 | ZNF644 | intron_06 | chr1: 90941310-90972743 | chr1: 90970361-90970380 | AGCUGUGCUUCAGAUAAACU | + |
| 1985 | 84146 | ZNF644 | intron_06 | chr1: 90941310-90972743 | chr1: 90970254-90970273 | CAAUAGAAAACCUGUAUUCA | + |
| 1986 | 84146 | ZNF644 | intron_06 | chr1: 90941310-90972743 | chr1: 90969822-90969841 | GAGGUUUUCGCAUCCACUGG | + |
| 1987 | 84146 | ZNF644 | intron_06 | chr1: 90941310-90972743 | chr1: 90968924-90968943 | UAGAAUGUAAUAACAAGAUG | − |
| 1988 | 84146 | ZNF644 | intron_06 | chr1: 90941310-90972743 | chr1: 90968874-90968893 | GUUUGCUGUGGCAAGUAAAU | − |
| 1989 | 84146 | ZNF644 | intron_06 | chr1: 90941310-90972743 | chr1: 90968751-90968770 | UUCUUUUCCUGAAUACUUUA | + |
| 1990 | 84146 | ZNF644 | intron_06 | chr1: 90941310-90972743 | chr1: 90968761-90968780 | AAGGUAUCCUUAAAGUAUUC | − |
| 1991 | 84146 | ZNF644 | intron_06 | chr1: 90941310-90972743 | chr1: 90968413-90968432 | CUAAUAUAUUUCGGUUUUGG | + |
| 1992 | 84146 | ZNF644 | intron_06 | chr1: 90941310-90972743 | chr1: 90968412-90968431 | UCUAAUAUAUUUCGGUUUUG | + |
| 1993 | 84146 | ZNF644 | intron_06 | chr1: 90941310-90972743 | chr1: 90968411-90968430 | UUCUAAUAUAUUUCGGUUUU | + |
| 1994 | 84146 | ZNF644 | intron_06 | chr1: 90941310-90972743 | chr1: 90968322-90968341 | GUCAAAAUAUAGUAUUGCAU | + |
| 1995 | 84146 | ZNF644 | intron_06 | chr1: 90941310-90972743 | chr1: 90968295-90968314 | UAUUGUAAAUUAUAUCUCAU | − |
| 1996 | 84146 | ZNF644 | intron_06 | chr1: 90941310-90972743 | chr1: 90968248-90968267 | AUCAGUACAUUUACAUACCU | − |
| 1997 | 84146 | ZNF644 | intron_06 | chr1: 90941310-90972743 | chr1: 90968206-90968225 | GUUAUUUUGUAGGAAUUUGC | + |
| 1998 | 84146 | ZNF644 | intron_06 | chr1: 90941310-90972743 | chr1: 90968198-90968217 | CUACAAAAUAACUGAUCAUG | − |
| 1999 | 84146 | ZNF644 | intron_06 | chr1: 90941310-90972743 | chr1: 90968163-90968182 | GUAUAUCACUAAUUGAGAUA | − |
| 2000 | 84146 | ZNF644 | intron_06 | chr1: 90941310-90972743 | chr1: 90967735-90967754 | UCUUUUUGUGCACUCACUGG | − |
| 2001 | 84146 | ZNF644 | intron_06 | chr1: 90941310-90972743 | chr1: 90967729-90967748 | UGUGCACUCACUGGUGGCUA | − |
| 2002 | 84146 | ZNF644 | intron_06 | chr1: 90941310-90972743 | chr1: 90967728-90967747 | GUGCACUCACUGGUGGCUAG | − |
| 2003 | 84146 | ZNF644 | intron_06 | chr1: 90941310-90972743 | chr1: 90967724-90967743 | ACUCACUGGUGGCUAGGGGC | − |
| 2004 | 84146 | ZNF644 | intron_06 | chr1: 90941310-90972743 | chr1: 90967671-90967690 | AACCAGUUGAAGUUAAUAAA | − |
| 2005 | 84146 | ZNF644 | intron_06 | chr1: 90941310-90972743 | chr1: 90967625-90967644 | CCCUGAUAUCAGUCAAUUAU | + |
| 2006 | 84146 | ZNF644 | intron_06 | chr1: 90941310-90972743 | chr1: 90967628-90967647 | CCUAUAAUUGACUGAUAUCA | − |
| 2007 | 84146 | ZNF644 | intron_06 | chr1: 90941310-90972743 | chr1: 90967580-90967599 | GUUGUCUGUGUUCCUUCAUU | − |
| 2008 | 84146 | ZNF644 | intron_06 | chr1: 90941310-90972743 | chr1: 90967544-90967563 | UGCUGUUUUGAACAACUAAA | + |
| 2009 | 84146 | ZNF644 | intron_06 | chr1: 90941310-90972743 | chr1: 90967509-90967528 | AGCAUAUGCAAUAGAAGACA | − |
| 2010 | 84146 | ZNF644 | intron_06 | chr1: 90941310-90972743 | chr1: 90967484-90967503 | CCCAUUCUCUUGGAAUCACA | − |
| 2011 | 84146 | ZNF644 | intron_06 | chr1: 90941310-90972743 | chr1: 90967442-90967461 | UUUUGGAACUUAGGACCAUU | − |
| 2012 | 84146 | ZNF644 | intron_06 | chr1: 90941310-90972743 | chr1: 90967364-90967383 | UGACAAAUGCUCUUACAGAA | + |
| 2013 | 84146 | ZNF644 | intron_06 | chr1: 90941310-90972743 | chr1: 90967346-90967365 | CAAAGGAACUUGACACAGAG | − |
| 2014 | 84146 | ZNF644 | intron_06 | chr1: 90941310-90972743 | chr1: 90967191-90967210 | ACAUGUUCCUCUUCACCAUA | + |
| 2015 | 84146 | ZNF644 | intron_06 | chr1: 90941310-90972743 | chr1: 90967071-90967090 | AAUCUUUUAUACAGUUGAAA | + |
| 2016 | 84146 | ZNF644 | intron_06 | chr1: 90941310-90972743 | chr1: 90966987-90967006 | CUAAUCAUUUUCUGCACCAC | + |
| 2017 | 84146 | ZNF644 | intron_06 | chr1: 90941310-90972743 | chr1: 90966853-90966872 | UCACGUGAUAGGAGCACUUU | + |
| 2018 | 84146 | ZNF644 | intron_06 | chr1: 90941310-90972743 | chr1: 90966852-90966871 | CUCACGUGAUAGGAGCACUU | + |
| 2019 | 84146 | ZNF644 | intron_06 | chr1: 90941310-90972743 | chr1: 90966086-90966105 | ACUAUUUCUCGACUAUGCUC | + |
| 2020 | 84146 | ZNF644 | intron_06 | chr1: 90941310-90972743 | chr1: 90965481-90965500 | AGUCUGAAGUAGAGGCGGAC | + |
| 2021 | 84146 | ZNF644 | intron_06 | chr1: 90941310-90972743 | chr1: 90965280-90965299 | AUCUGUUUCAGAUUUCUUCU | + |
| 2022 | 84146 | ZNF644 | intron_06 | chr1: 90941310-90972743 | chr1: 90965159-90965178 | UAACAAAGUCCUGCAAGUGG | + |
| 2023 | 84146 | ZNF644 | intron_06 | chr1: 90941310-90972743 | chr1: 90965156-90965175 | UUUUAACAAAGUCCUGCAAG | + |
| 2024 | 84146 | ZNF644 | intron_06 | chr1: 90941310-90972743 | chr1: 90964945-90964964 | AAAGCAACUUAUGUCUUCUA | − |
| 2025 | 84146 | ZNF644 | intron_06 | chr1: 90941310-90972743 | chr1: 90964785-90964804 | AUAGAAGAGGUCUUUUUUUU | + |
| 2026 | 84146 | ZNF644 | intron_06 | chr1: 90941310-90972743 | chr1: 90964694-90964713 | GCUAUACUGGAAUAUUAUAC | + |
| 2027 | 84146 | ZNF644 | intron_06 | chr1: 90941310-90972743 | chr1: 90964394-90964413 | UUUUGAUAGAAACCUCCUUU | − |
| 2028 | 84146 | ZNF644 | intron_06 | chr1: 90941310-90972743 | chr1: 90964337-90964356 | UUCUCACAUUUAGACAAAGA | + |
| 2029 | 84146 | ZNF644 | intron_06 | chr1: 90941310-90972743 | chr1: 90963978-90963997 | GUGUAAUGUAUUGUUUUCUA | − |
| 2030 | 84146 | ZNF644 | intron_06 | chr1: 90941310-90972743 | chr1: 90963848-90963867 | GUCAUGGAGUGUUUACUUUG | + |
| 2031 | 84146 | ZNF644 | intron_06 | chr1: 90941310-90972743 | chr1: 90963798-90963817 | AUAGGGGGUUUCUGGGUUGC | + |
| 2032 | 84146 | ZNF644 | intron_06 | chr1: 90941310-90972743 | chr1: 90963791-90963810 | AAGAGACAUAGGGGGUUUCU | + |
| 2033 | 84146 | ZNF644 | intron_06 | chr1: 90941310-90972743 | chr1: 90963739-90963758 | GUCAGGCAAGUGGUUAACCU | + |
| 2034 | 84146 | ZNF644 | intron_06 | chr1: 90941310-90972743 | chr1: 90963738-90963757 | AGUCAGGCAAGUGGUUAACC | + |
| 2035 | 84146 | ZNF644 | intron_06 | chr1: 90941310-90972743 | chr1: 90963566-90963585 | AUUUGUCAUGUUACUGUAGG | − |
| 2036 | 84146 | ZNF644 | intron_06 | chr1: 90941310-90972743 | chr1: 90963561-90963580 | UCAUGUUACUGUAGGAGGUU | − |
| 2037 | 84146 | ZNF644 | intron_06 | chr1: 90941310-90972743 | chr1: 90963560-90963579 | CAUGUUACUGUAGGAGGUUU | − |
| 2038 | 84146 | ZNF644 | intron_06 | chr1: 90941310-90972743 | chr1: 90963460-90963479 | AAUGACCCAGAUUCUAGUCU | + |
| 2039 | 84146 | ZNF644 | intron_06 | chr1: 90941310-90972743 | chr1: 90963304-90963323 | UCAGAAGACUAAAAUUAAAG | + |
| 2040 | 84146 | ZNF644 | intron_06 | chr1: 90941310-90972743 | chr1: 90963263-90963282 | AAAGGGUAUUCAAUUCCAUU | + |
| 2041 | 84146 | ZNF644 | intron_06 | chr1: 90941310-90972743 | chr1: 90962834-90962853 | AAACUAUGCUUCUCUACUAG | − |
| 2042 | 84146 | ZNF644 | intron_06 | chr1: 90941310-90972743 | chr1: 90962803-90962822 | AGAUGUGUGGUUAGAGAGGA | + |
| 2043 | 84146 | ZNF644 | intron_06 | chr1: 90941310-90972743 | chr1: 90962799-90962818 | UGGGAGAUGUGUGGUUAGAG | + |
| 2044 | 84146 | ZNF644 | intron_06 | chr1: 90941310-90972743 | chr1: 90962779-90962798 | CAAAUGUCCCACCCAGAUGG | + |
| 2045 | 84146 | ZNF644 | intron_06 | chr1: 90941310-90972743 | chr1: 90962790-90962809 | CACAUCUCCCACCAUCUGGG | − |
| 2046 | 84146 | ZNF644 | intron_06 | chr1: 90941310-90972743 | chr1: 90962789-90962808 | ACAUCUCCCACCAUCUGGGU | − |
| 2047 | 84146 | ZNF644 | intron_06 | chr1: 90941310-90972743 | chr1: 90962667-90962686 | CUUAGCAUUCUGUACAUUGU | + |
| 2048 | 84146 | ZNF644 | intron_06 | chr1: 90941310-90972743 | chr1: 90962440-90962459 | AUACAUUCACAUGUAAGAAA | + |
| 2049 | 84146 | ZNF644 | intron_06 | chr1: 90941310-90972743 | chr1: 90962301-90962320 | AGACAAAAGUAGACCAAUGG | + |
| 2050 | 84146 | ZNF644 | intron_06 | chr1: 90941310-90972743 | chr1: 90962298-90962317 | GAUAGACAAAAGUAGACCAA | + |
| 2051 | 84146 | ZNF644 | intron_06 | chr1: 90941310-90972743 | chr1: 90962173-90962192 | CUGAUAUGAAUAAAAAUUUA | + |
| 2052 | 84146 | ZNF644 | intron_06 | chr1: 90941310-90972743 | chr1: 90962067-90962086 | GUCUGAGUGAGCUAAAUAAA | + |
| 2053 | 84146 | ZNF644 | intron_06 | chr1: 90941310-90972743 | chr1: 90961918-90961937 | CCACUGAACAGAAAAUUAUC | + |
| 2054 | 84146 | ZNF644 | intron_06 | chr1: 90941310-90972743 | chr1: 90961833-90961852 | UCACACUGGGUAAGUACUAC | + |
| 2055 | 84146 | ZNF644 | intron_06 | chr1: 90941310-90972743 | chr1: 90961771-90961790 | CAGUAAAUAUUGUUCUCAAC | + |
| 2056 | 84146 | ZNF644 | intron_06 | chr1: 90941310-90972743 | chr1: 90961394-90961413 | UGUGAGUUGUGGGGCCCAGG | + |
| 2057 | 84146 | ZNF644 | intron_06 | chr1: 90941310-90972743 | chr1: 90961392-90961411 | AGUGUGAGUUGUGGGGCCCA | + |
| 2058 | 84146 | ZNF644 | intron_06 | chr1: 90941310-90972743 | chr1: 90961391-90961410 | AAGUGUGAGUUGUGGGGCCC | + |
| 2059 | 84146 | ZNF644 | intron_06 | chr1: 90941310-90972743 | chr1: 90961385-90961404 | UCUGAAAAGUGUGAGUUGUG | + |
| 2060 | 84146 | ZNF644 | intron_06 | chr1: 90941310-90972743 | chr1: 90961384-90961403 | GUCUGAAAAGUGUGAGUUGU | + |
| 2061 | 84146 | ZNF644 | intron_06 | chr1: 90941310-90972743 | chr1: 90961383-90961402 | GGUCUGAAAAGUGUGAGUUG | + |
| 2062 | 84146 | ZNF644 | intron_06 | chr1: 90941310-90972743 | chr1: 90961362-90961381 | GCUGUGAUCUCUAGGACAUG | + |
| 2063 | 84146 | ZNF644 | intron_06 | chr1: 90941310-90972743 | chr1: 90961201-90961220 | GUUGAGUAAGGGGUACAGUA | + |
| 2064 | 84146 | ZNF644 | intron_06 | chr1: 90941310-90972743 | chr1: 90961078-90961097 | AGAAUAAAAAGCGAUACAAG | + |
| 2065 | 84146 | ZNF644 | intron_06 | chr1: 90941310-90972743 | chr1: 90960797-90960816 | UUGCUAGUCUUUUUGGAUGG | + |
| 2066 | 84146 | ZNF644 | intron_06 | chr1: 90941310-90972743 | chr1: 90960790-90960809 | GGUGUUCUUGCUAGUCUUUU | + |
| 2067 | 84146 | ZNF644 | intron_06 | chr1: 90941310-90972743 | chr1: 90960769-90960788 | UAAAAAAUGUGCUCAACUAC | + |
| 2068 | 84146 | ZNF644 | intron_06 | chr1: 90941310-90972743 | chr1: 90960341-90960360 | GUGGUGCAUUUUAAGAGCUU | + |
| 2069 | 84146 | ZNF644 | intron_06 | chr1: 90941310-90972743 | chr1: 90960254-90960273 | AAAUCUUCUACUCGCUGAAU | + |
| 2070 | 84146 | ZNF644 | intron_06 | chr1: 90941310-90972743 | chr1: 90959775-90959794 | UUUAGAAACUAGAGAGCUAG | + |
| 2071 | 84146 | ZNF644 | intron_06 | chr1: 90941310-90972743 | chr1: 90959692-90959711 | UUACUUUACCAGGUGAUGGA | + |
| 2072 | 84146 | ZNF644 | intron_06 | chr1: 90941310-90972743 | chr1: 90959688-90959707 | AAGGUUACUUUACCAGGUGA | + |
| 2073 | 84146 | ZNF644 | intron_06 | chr1: 90941310-90972743 | chr1: 90958861-90958880 | AGCUCUCAGAAGGCAUGGGU | + |
| 2074 | 84146 | ZNF644 | intron_06 | chr1: 90941310-90972743 | chr1: 90957182-90957201 | CCAGGACUAUGCGGUUUCAC | + |
| 2075 | 84146 | ZNF644 | intron_06 | chr1: 90941310-90972743 | chr1: 90956942-90956961 | UGAAAAAUCAGGAAUAGAAG | + |
| 2076 | 84146 | ZNF644 | intron_06 | chr1: 90941310-90972743 | chr1: 90956605-90956624 | CCAUCUCACUUUAUGCCUUA | + |
| 2077 | 84146 | ZNF644 | intron_06 | chr1: 90941310-90972743 | chr1: 90956456-90956475 | ACAGAUGAGAUUUAGUAUGA | + |
| 2078 | 84146 | ZNF644 | intron_06 | chr1: 90941310-90972743 | chr1: 90956456-90956475 | UCAUACUAAAUCUCAUCUGU | − |
| 2079 | 84146 | ZNF644 | intron_06 | chr1: 90941310-90972743 | chr1: 90956455-90956474 | CAUACUAAAUCUCAUCUGUG | − |
| 2080 | 84146 | ZNF644 | intron_06 | chr1: 90941310-90972743 | chr1: 90956151-90956170 | CUUGGAUUGGGCAUGGUUUG | + |
| 2081 | 84146 | ZNF644 | intron_06 | chr1: 90941310-90972743 | chr1: 90956144-90956163 | UCACUAUCUUGGAUUGGGCA | + |
| 2082 | 84146 | ZNF644 | intron_06 | chr1: 90941310-90972743 | chr1: 90956133-90956152 | AUCAAUUCAGUUCACUAUCU | + |
| 2083 | 84146 | ZNF644 | intron_06 | chr1: 90941310-90972743 | chr1: 90955856-90955875 | CAAACUUGGCUAAAUGUUUG | + |
| 2084 | 84146 | ZNF644 | intron_06 | chr1: 90941310-90972743 | chr1: 90955446-90955465 | AUCUACACUGAAAAAUCUGU | + |
| 2085 | 84146 | ZNF644 | intron_06 | chr1: 90941310-90972743 | chr1: 90955341-90955360 | AGAGGGUCCGCCUGUCUCUC | + |
| 2086 | 84146 | ZNF644 | intron_06 | chr1: 90941310-90972743 | chr1: 90954981-90955000 | AAAUGUACUUAGUGACUUCA | + |
| 2087 | 84146 | ZNF644 | intron_06 | chr1: 90941310-90972743 | chr1: 90953375-90953394 | CCUAUGGGGUAGGGGGUGAA | + |
| 2088 | 84146 | ZNF644 | intron_06 | chr1: 90941310-90972743 | chr1: 90953350-90953369 | ACAUGGAGAGGGGAACACAC | + |
| 2089 | 84146 | ZNF644 | intron_06 | chr1: 90941310-90972743 | chr1: 90953340-90953359 | CAUUUAACAGACAUGGAGAG | + |
| 2090 | 84146 | ZNF644 | intron_06 | chr1: 90941310-90972743 | chr1: 90953201-90953220 | UCACCUUCUUUACCAAUUAC | + |
| 2091 | 84146 | ZNF644 | intron_06 | chr1: 90941310-90972743 | chr1: 90952896-90952915 | ACUACACUUAGAUACAUCAC | + |
| 2092 | 84146 | ZNF644 | intron_06 | chr1: 90941310-90972743 | chr1: 90952742-90952761 | CCAACAUCUAGUUGGAAUCC | + |
| 2093 | 84146 | ZNF644 | intron_06 | chr1: 90941310-90972743 | chr1: 90952648-90952667 | AGUAUAUUGAAUGAAGCAAG | + |
| 2094 | 84146 | ZNF644 | intron_06 | chr1: 90941310-90972743 | chr1: 90952553-90952572 | UCACUGUAGACCAUAUGCUC | + |
| 2095 | 84146 | ZNF644 | intron_06 | chr1: 90941310-90972743 | chr1: 90952424-90952443 | AGACCACUGAUCAAACAUAU | + |
| 2096 | 84146 | ZNF644 | intron_06 | chr1: 90941310-90972743 | chr1: 90952168-90952187 | UUACUGACAAAGGGUCACCU | + |
| 2097 | 84146 | ZNF644 | intron_06 | chr1: 90941310-90972743 | chr1: 90952159-90952178 | AAGCCAUUUUUACUGACAAA | + |
| 2098 | 84146 | ZNF644 | intron_06 | chr1: 90941310-90972743 | chr1: 90952084-90952103 | CUGAAUUUUAAUUCUAAGAC | − |
| 2099 | 84146 | ZNF644 | intron_06 | chr1: 90941310-90972743 | chr1: 90951946-90951965 | AUUGCAGCAAAGCACAUAAU | + |
| 2100 | 84146 | ZNF644 | intron_06 | chr1: 90941310-90972743 | chr1: 90951947-90951966 | UAUUAUGUGCUUUGCUGCAA | − |
| 2101 | 84146 | ZNF644 | intron_06 | chr1: 90941310-90972743 | chr1: 90951921-90951940 | GAGCUCCUUAGGCAGACAGA | + |
| 2102 | 84146 | ZNF644 | intron_06 | chr1: 90941310-90972743 | chr1: 90951806-90951825 | UUGCUAACUGAAUACCUCAA | + |
| 2103 | 84146 | ZNF644 | intron_06 | chr1: 90941310-90972743 | chr1: 90951527-90951546 | CAUGGCAAACUAUUUCAUCU | + |
| 2104 | 84146 | ZNF644 | intron_06 | chr1: 90941310-90972743 | chr1: 90951480-90951499 | AGCAUUGACUAUUCCUCAGA | + |
| 2105 | 84146 | ZNF644 | intron_06 | chr1: 90941310-90972743 | chr1: 90951153-90951172 | ACUUCAUCAGCUGACUAAUC | + |
| 2106 | 84146 | ZNF644 | intron_06 | chr1: 90941310-90972743 | chr1: 90950967-90950986 | AAGGCUGCUAGAUAUGUGUU | − |
| 2107 | 84146 | ZNF644 | intron_06 | chr1: 90941310-90972743 | chr1: 90950966-90950985 | AGGCUGCUAGAUAUGUGUUU | − |
| 2108 | 84146 | ZNF644 | intron_06 | chr1: 90941310-90972743 | chr1: 90950857-90950876 | CUCUUUCCCUAAAAUUGAUC | + |
| 2109 | 84146 | ZNF644 | intron_06 | chr1: 90941310-90972743 | chr1: 90950628-90950647 | AAAUCAACAGCCACAGAGGG | + |
| 2110 | 84146 | ZNF644 | intron_06 | chr1: 90941310-90972743 | chr1: 90949950-90949969 | AUGAACAACAAAAAGGAUUC | − |
| 2111 | 84146 | ZNF644 | intron_06 | chr1: 90941310-90972743 | chr1: 90949945-90949964 | CAACAAAAAGGAUUCAGGAA | − |
| 2112 | 84146 | ZNF644 | intron_06 | chr1: 90941310-90972743 | chr1: 90949938-90949957 | AAGGAUUCAGGAACGGUGUU | − |
| 2113 | 84146 | ZNF644 | intron_06 | chr1: 90941310-90972743 | chr1: 90949893-90949912 | GGAUUCCCAGUAUGUGGCUA | + |
| 2114 | 84146 | ZNF644 | intron_06 | chr1: 90941310-90972743 | chr1: 90949781-90949800 | UGGAGUUUGGUAUUCUUGCA | + |
| 2115 | 84146 | ZNF644 | intron_06 | chr1: 90941310-90972743 | chr1: 90949659-90949678 | UUUGGCAUUUACACUGUAGU | + |
| 2116 | 84146 | ZNF644 | intron_06 | chr1: 90941310-90972743 | chr1: 90949533-90949552 | AAUAUACUUGGCUCUCUCUG | + |
| 2117 | 84146 | ZNF644 | intron_06 | chr1: 90941310-90972743 | chr1: 90949427-90949446 | ACAAGUAUCUUAAUUUAGGA | + |
| 2118 | 84146 | ZNF644 | intron_06 | chr1: 90941310-90972743 | chr1: 90949375-90949394 | GAUGAUAAUUGGAUUUUUUC | − |
| 2119 | 84146 | ZNF644 | intron_06 | chr1: 90941310-90972743 | chr1: 90949057-90949076 | UUUACAAUUUAAUCUAUUGA | + |
| 2120 | 84146 | ZNF644 | intron_06 | chr1: 90941310-90972743 | chr1: 90948851-90948870 | UUUGAAAAACUUAAUUACUG | − |
| 2121 | 84146 | ZNF644 | intron_06 | chr1: 90941310-90972743 | chr1: 90948841-90948860 | UUAAUUACUGAGGUAUUAGA | − |
| 2122 | 84146 | ZNF644 | intron_06 | chr1: 90941310-90972743 | chr1: 90948791-90948810 | AGCAUGUCUAGCCUGAUGCU | + |
| 2123 | 84146 | ZNF644 | intron_06 | chr1: 90941310-90972743 | chr1: 90948786-90948805 | CAGGCUAGACAUGCUGGUAA | − |
| 2124 | 84146 | ZNF644 | intron_06 | chr1: 90941310-90972743 | chr1: 90948491-90948510 | GAUUGGUUCUAAAAACCUGG | + |
| 2125 | 84146 | ZNF644 | intron_06 | chr1: 90941310-90972743 | chr1: 90948488-90948507 | GCUGAUUGGUUCUAAAAACC | + |
| 2126 | 84146 | ZNF644 | intron_06 | chr1: 90941310-90972743 | chr1: 90948434-90948453 | GCCUUAACAGAUUGUUAAUG | + |
| 2127 | 84146 | ZNF644 | intron_06 | chr1: 90941310-90972743 | chr1: 90948370-90948389 | GACUGCAAAUGCAAACAUGG | + |
| 2128 | 84146 | ZNF644 | intron_06 | chr1: 90941310-90972743 | chr1: 90948367-90948386 | CAUGACUGCAAAUGCAAACA | + |
| 2129 | 84146 | ZNF644 | intron_06 | chr1: 90941310-90972743 | chr1: 90948344-90948363 | AGUAUGCACAACAAAGUUAG | + |
| 2130 | 84146 | ZNF644 | intron_06 | chr1: 90941310-90972743 | chr1: 90948313-90948332 | AUGUAUAUUCCACAGGAUCA | + |
| 2131 | 84146 | ZNF644 | intron_06 | chr1: 90941310-90972743 | chr1: 90948314-90948333 | UUGAUCCUGUGGAAUAUACA | − |
| 2132 | 84146 | ZNF644 | intron_06 | chr1: 90941310-90972743 | chr1: 90948203-90948222 | AAGCAAAUAGAUAUCUGACU | − |
| 2133 | 84146 | ZNF644 | intron_06 | chr1: 90941310-90972743 | chr1: 90948130-90948149 | AUAAUUGUUGUGAAAUUUAU | − |
| 2134 | 84146 | ZNF644 | intron_06 | chr1: 90941310-90972743 | chr1: 90947959-90947978 | AAUCAUUGUCAUUGUAAGUC | + |
| 2135 | 84146 | ZNF644 | intron_06 | chr1: 90941310-90972743 | chr1: 90947667-90947686 | GUGAGAGUGUUCAAAGUCCU | + |
| 2136 | 84146 | ZNF644 | intron_06 | chr1: 90941310-90972743 | chr1: 90947525-90947544 | UGGCAUAAUACUUUCUGUGG | − |
| 2137 | 84146 | ZNF644 | intron_06 | chr1: 90941310-90972743 | chr1: 90947481-90947500 | UAUGUCAAUCCCAUACUCCA | + |
| 2138 | 84146 | ZNF644 | intron_06 | chr1: 90941310-90972743 | chr1: 90947480-90947499 | GUAUGUCAAUCCCAUACUCC | + |
| 2139 | 84146 | ZNF644 | intron_06 | chr1: 90941310-90972743 | chr1: 90947386-90947405 | UUGACCACAUUUGUGUAACA | + |
| 2140 | 84146 | ZNF644 | intron_06 | chr1: 90941310-90972743 | chr1: 90947201-90947220 | UAUGACUUAGUACUCUCCUU | − |
| 2141 | 84146 | ZNF644 | intron_06 | chr1: 90941310-90972743 | chr1: 90947182-90947201 | UAAAUUACAGCUGCAACCAA | + |
| 2142 | 84146 | ZNF644 | intron_06 | chr1: 90941310-90972743 | chr1: 90947149-90947168 | CUAAUGAAGAAUUCUGCAAC | + |
| 2143 | 84146 | ZNF644 | intron_06 | chr1: 90941310-90972743 | chr1: 90947052-90947071 | UUUAAAGUUAUGUCUUUGUA | − |
| 2144 | 84146 | ZNF644 | intron_06 | chr1: 90941310-90972743 | chr1: 90947017-90947036 | CUGUGUGGAUGAAGGCAAGG | + |
| 2145 | 84146 | ZNF644 | intron_06 | chr1: 90941310-90972743 | chr1: 90947014-90947033 | UAACUGUGUGGAUGAAGGCA | + |
| 2146 | 84146 | ZNF644 | intron_06 | chr1: 90941310-90972743 | chr1: 90946911-90946930 | CUUGAGUUAUUAGGUGUUUA | − |
| 2147 | 84146 | ZNF644 | intron_06 | chr1: 90941310-90972743 | chr1: 90946846-90946865 | UGAGGAUGGUACAUGGCAUC | − |
| 2148 | 84146 | ZNF644 | intron_06 | chr1: 90941310-90972743 | chr1: 90946592-90946611 | GUUAACUAAAGGAUUAUUUC | − |
| 2149 | 84146 | ZNF644 | intron_06 | chr1: 90941310-90972743 | chr1: 90946407-90946426 | CAGACAGGAAUAUGGAGUUG | + |
| 2150 | 84146 | ZNF644 | intron_06 | chr1: 90941310-90972743 | chr1: 90946392-90946411 | UUGUCAAGAACAUUACAGAC | + |
| 2151 | 84146 | ZNF644 | intron_06 | chr1: 90941310-90972743 | chr1: 90946352-90946371 | AACCAUUUUAUUAUUUGGCU | − |
| 2152 | 84146 | ZNF644 | intron_06 | chr1: 90941310-90972743 | chr1: 90946219-90946238 | CUUCUCUUGAAGUGUGCACU | − |
| 2153 | 84146 | ZNF644 | intron_06 | chr1: 90941310-90972743 | chr1: 90945811-90945830 | CAAGUAGGUUAACAUUUUGU | + |
| 2154 | 84146 | ZNF644 | intron_06 | chr1: 90941310-90972743 | chr1: 90945810-90945829 | GCAAGUAGGUUAACAUUUUG | + |
| 2155 | 84146 | ZNF644 | intron_06 | chr1: 90941310-90972743 | chr1: 90945796-90945815 | UAAUUGUAUUUAUUGCAAGU | + |
| 2156 | 84146 | ZNF644 | intron_06 | chr1: 90941310-90972743 | chr1: 90945669-90945688 | UCUGAAUUACUGAGUGACCU | − |
| 2157 | 84146 | ZNF644 | intron_06 | chr1: 90941310-90972743 | chr1: 90945653-90945672 | ACCUAGGAAGCUUGCUCAGA | − |
| 2158 | 84146 | ZNF644 | intron_06 | chr1: 90941310-90972743 | chr1: 90945436-90945455 | GCACUAAAAAAAUUGUACAA | + |
| 2159 | 84146 | ZNF644 | intron_06 | chr1: 90941310-90972743 | chr1: 90945419-90945438 | UGCUCACAGUAUAUAACUAG | − |
| 2160 | 84146 | ZNF644 | intron_06 | chr1: 90941310-90972743 | chr1: 90945259-90945278 | UAUGGUUAUAUGAUUAUAUG | − |
| 2161 | 84146 | ZNF644 | intron_06 | chr1: 90941310-90972743 | chr1: 90945052-90945071 | AUAUACAUUUGACUAUACAA | − |
| 2162 | 84146 | ZNF644 | intron_06 | chr1: 90941310-90972743 | chr1: 90945028-90945047 | AGUUGAAAAACUAUUUGAGA | − |
| 2163 | 84146 | ZNF644 | intron_06 | chr1: 90941310-90972743 | chr1: 90944970-90944989 | UGUUGGGUUAUGUGGCAUCU | + |
| 2164 | 84146 | ZNF644 | intron_06 | chr1: 90941310-90972743 | chr1: 90944894-90944913 | CAACAGAUUUUGAUGAUCCA | − |
| 2165 | 84146 | ZNF644 | intron_06 | chr1: 90941310-90972743 | chr1: 90944745-90944764 | CAAGUAGAUUAACGUUUUGU | + |
| 2166 | 84146 | ZNF644 | intron_06 | chr1: 90941310-90972743 | chr1: 90944662-90944681 | AAUUACUCAUGAACAGAAAA | − |
| 2167 | 84146 | ZNF644 | intron_06 | chr1: 90941310-90972743 | chr1: 90944591-90944610 | AGGAAGCUUUCUAGGUCACU | + |
| 2168 | 84146 | ZNF644 | intron_06 | chr1: 90941310-90972743 | chr1: 90944583-90944602 | GUUGACUUAGGAAGCUUUCU | + |
| 2169 | 84146 | ZNF644 | intron_06 | chr1: 90941310-90972743 | chr1: 90944474-90944493 | AUUUUAUUAUGUGGAUGUGA | − |
| 2170 | 84146 | ZNF644 | intron_06 | chr1: 90941310-90972743 | chr1: 90944267-90944286 | GGAGACUGCAUCCUCAUUAC | + |
| 2171 | 84146 | ZNF644 | intron_06 | chr1: 90941310-90972743 | chr1: 90944246-90944265 | AUGAGAACAUCAUGUCAGCU | + |
| 2172 | 84146 | ZNF644 | intron_06 | chr1: 90941310-90972743 | chr1: 90944161-90944180 | UGGUGUUAGAGGUUUAGAGA | + |
| 2173 | 84146 | ZNF644 | intron_06 | chr1: 90941310-90972743 | chr1: 90944150-90944169 | CCCAUCUGUUCUGGUGUUAG | + |
| 2174 | 84146 | ZNF644 | intron_06 | chr1: 90941310-90972743 | chr1: 90944141-90944160 | UAUGUUCCACCCAUCUGUUC | + |
| 2175 | 84146 | ZNF644 | intron_06 | chr1: 90941310-90972743 | chr1: 90944153-90944172 | CCUCUAACACCAGAACAGAU | − |
| 2176 | 84146 | ZNF644 | intron_06 | chr1: 90941310-90972743 | chr1: 90944150-90944169 | CUAACACCAGAACAGAUGGG | − |
| 2177 | 84146 | ZNF644 | intron_06 | chr1: 90941310-90972743 | chr1: 90944136-90944155 | GAUGGGUGGAACAUAGAACA | − |
| 2178 | 84146 | ZNF644 | intron_06 | chr1: 90941310-90972743 | chr1: 90944115-90944134 | GGAAGGUUUUACCUCUGAGG | − |
| 2179 | 84146 | ZNF644 | intron_06 | chr1: 90941310-90972743 | chr1: 90944078-90944097 | GUAAAUUCAGUGUUAAUGAG | + |
| 2180 | 84146 | ZNF644 | intron_06 | chr1: 90941310-90972743 | chr1: 90944056-90944075 | AAGCAAUGUUGCUAACGAAG | + |
| 2181 | 84146 | ZNF644 | intron_06 | chr1: 90941310-90972743 | chr1: 90943981-90944000 | ACUUAUUAUAAUCACAGAGG | + |
| 2182 | 84146 | ZNF644 | intron_06 | chr1: 90941310-90972743 | chr1: 90943971-90943990 | UAUAAUAAGUGGGAACGGAA | − |
| 2183 | 84146 | ZNF644 | intron_06 | chr1: 90941310-90972743 | chr1: 90943799-90943818 | AAAGGGUGUGCAAAUGUCAA | + |
| 2184 | 84146 | ZNF644 | intron_06 | chr1: 90941310-90972743 | chr1: 90943782-90943801 | CAUAAACAUGUGUUAGGAAA | + |
| 2185 | 84146 | ZNF644 | intron_06 | chr1: 90941310-90972743 | chr1: 90943781-90943800 | ACAUAAACAUGUGUUAGGAA | + |
| 2186 | 84146 | ZNF644 | intron_06 | chr1: 90941310-90972743 | chr1: 90943713-90943732 | GUGAUGCAUUAGCACCAUUU | − |
| 2187 | 84146 | ZNF644 | intron_06 | chr1: 90941310-90972743 | chr1: 90943642-90943661 | UUGAGAUUGGCAAACUUCUG | + |
| 2188 | 84146 | ZNF644 | intron_06 | chr1: 90941310-90972743 | chr1: 90943629-90943648 | UUAGAAUUCUGUUUUGAGAU | + |
| 2189 | 84146 | ZNF644 | intron_06 | chr1: 90941310-90972743 | chr1: 90943619-90943638 | AGAAUUCUAAAACCUACGAG | − |
| 2190 | 84146 | ZNF644 | intron_06 | chr1: 90941310-90972743 | chr1: 90943093-90943112 | CAUGAAAUUGGUACCCUUAA | − |
| 2191 | 84146 | ZNF644 | intron_06 | chr1: 90941310-90972743 | chr1: 90943033-90943052 | UUUCUAAAUGUUAGAUUGUA | − |
| 2192 | 84146 | ZNF644 | intron_06 | chr1: 90941310-90972743 | chr1: 90943006-90943025 | UAAUACUAUUUUAGGGCAAC | + |
| 2193 | 84146 | ZNF644 | intron_06 | chr1: 90941310-90972743 | chr1: 90942895-90942914 | UUCCUGUAUUCCAGAUAUAA | + |
| 2194 | 84146 | ZNF644 | intron_06 | chr1: 90941310-90972743 | chr1: 90942790-90942809 | GUAUUAACUAAUUGAAGUAC | + |
| 2195 | 84146 | ZNF644 | intron_06 | chr1: 90941310-90972743 | chr1: 90942788-90942807 | ACUUCAAUUAGUUAAUACCU | − |
| 2196 | 84146 | ZNF644 | intron_06 | chr1: 90941310-90972743 | chr1: 90942772-90942791 | ACCUGGGGUUGCCAAAUAGC | − |
| 2197 | 84146 | ZNF644 | intron_06 | chr1: 90941310-90972743 | chr1: 90942673-90942692 | UGAUGGAACUUGUGUUUGAA | − |
| 2198 | 84146 | ZNF644 | intron_06 | chr1: 90941310-90972743 | chr1: 90942642-90942661 | AGUUUUGUUCCUAUGAAAUU | + |
| 2199 | 84146 | ZNF644 | intron_06 | chr1: 90941310-90972743 | chr1: 90942654-90942673 | AUGGGGUAUCCAAAUUUCAU | − |
| 2200 | 84146 | ZNF644 | intron_06 | chr1: 90941310-90972743 | chr1: 90942521-90942540 | AUCACUUGCCAUAGGUGCUC | + |
| 2201 | 84146 | ZNF644 | intron_06 | chr1: 90941310-90972743 | chr1: 90942513-90942532 | CCUAGUACAUCACUUGCCAU | + |
| 2202 | 84146 | ZNF644 | intron_06 | chr1: 90941310-90972743 | chr1: 90942413-90942432 | GCUCUUAGAAAUGGCCACAU | + |
| 2203 | 84146 | ZNF644 | intron_06 | chr1: 90941310-90972743 | chr1: 90942413-90942432 | AUGUGGCCAUUUCUAAGAGC | − |
| 2204 | 84146 | ZNF644 | intron_06 | chr1: 90941310-90972743 | chr1: 90942393-90942412 | AGGAAAAAAACAGGUUGCAA | − |
| 2205 | 84146 | ZNF644 | intron_06 | chr1: 90941310-90972743 | chr1: 90942376-90942395 | CAAGGGAACUUUUCAUAGUG | − |
| 2206 | 84146 | ZNF644 | intron_06 | chr1: 90941310-90972743 | chr1: 90942155-90942174 | CUAGACCACCAGAUCAUAAG | − |
| 2207 | 84146 | ZNF644 | intron_06 | chr1: 90941310-90972743 | chr1: 90942108-90942127 | UAUGUGGGGCCUCAAAUUCU | + |
| 2208 | 84146 | ZNF644 | intron_06 | chr1: 90941310-90972743 | chr1: 90942094-90942113 | AUGUCAAACUUUCAUAUGUG | + |
| 2209 | 84146 | ZNF644 | intron_06 | chr1: 90941310-90972743 | chr1: 90941944-90941963 | UUUUACAAUUGAUAGAAUGU | − |
| 2210 | 84146 | ZNF644 | intron_06 | chr1: 90941310-90972743 | chr1: 90941943-90941962 | UUUACAAUUGAUAGAAUGUU | − |
| 2211 | 84146 | ZNF644 | intron_06 | chr1: 90941310-90972743 | chr1: 90941830-90941849 | CAAUAAAGUUAUUUACUGAA | − |
| 2212 | 84146 | ZNF644 | intron_06 | chr1: 90941310-90972743 | chr1: 90941794-90941813 | ACUUAUAAGUUCAUUCCUGA | + |
| 2213 | 84146 | ZNF644 | intron_06 | chr1: 90941310-90972743 | chr1: 90941696-90941715 | GAAAAUUCUUUCACUUAAGU | + |
| 2214 | 84146 | ZNF644 | intron_06 | chr1: 90941310-90972743 | chr1: 90941564-90941583 | GCAACAUACACAAACACACU | + |
| 2215 | 84146 | ZNF644 | intron_06 | chr1: 90941310-90972743 | chr1: 90941493-90941512 | UUAAUAUUGUUCUUCAUGCU | + |
| 2216 | 84146 | ZNF644 | intron_06 | chr1: 90941310-90972743 | chr1: 90941350-90941369 | AAAAAUACUAUUAGAAUGUA | + |
| 2217 | 84146 | ZNF644 | exon_07_c.1/nc.1 | chr1: 90940949-90941309 | chr1: 90941278-90941297 | AAAUGGGCUUGCCAACAAUA | − |
| 2218 | 84146 | ZNF644 | exon_07_c.1/nc.1 | chr1: 90940949-90941309 | chr1: 90941193-90941212 | UUUAUCUCAGACAAAGAGAG | − |
| 2219 | 84146 | ZNF644 | exon_07_c.1/nc.1 | chr1: 90940949-90941309 | chr1: 90941157-90941176 | AAUGAUGUUUGACAAUCUUU | + |
| 2220 | 84146 | ZNF644 | exon_07_c.1/nc.1 | chr1: 90940949-90941309 | chr1: 90941116-90941135 | UCUGCCUGAAGAACUGUCAA | − |
| 2221 | 84146 | ZNF644 | exon_07_c.1/nc.1 | chr1: 90940949-90941309 | chr1: 90941075-90941094 | ACUAGACUGGCCUCCACUUA | + |
| 2222 | 84146 | ZNF644 | exon_07_c.1/nc.1 | chr1: 90940949-90941309 | chr1: 90941062-90941081 | CAUGUAUAAAUAGACUAGAC | + |
| 2223 | 84146 | ZNF644 | exon_07_c.1/nc.1 | chr1: 90940949-90941309 | chr1: 90940977-90940996 | GUUAAGGAGGAGUGUGAAAC | + |
| 2224 | 84146 | ZNF644 | exon_07_c.1/nc.1 | chr1: 90940949-90941309 | chr1: 90940961-90940980 | CAUAUUGGAAGUCUUAGUUA | + |
| 2225 | 84146 | ZNF644 | exon_07_c.1/ | chr1: 90940121-90940948 | chr1: 90940888-90940907 | AGAAUCUUGUUCAACUUUGA | − |
| 2226 | 84146 | ZNF644 | exon_07_c.1/ | chr1: 90940121-90940948 | chr1: 90940836-90940855 | UGGUGUUGACUUGCUUUCUG | + |
| 2227 | 84146 | ZNF644 | exon_07_c.1/ | chr1: 90940121-90940948 | chr1: 90940768-90940787 | UAGGUAGUUUUUUAUUGGAA | + |
| 2228 | 84146 | ZNF644 | exon_07_c.1/ | chr1: 90940121-90940948 | chr1: 90940749-90940768 | CAACCAACUGAAGCAGAGGU | + |
| 2229 | 84146 | ZNF644 | exon_07_c.1/ | chr1: 90940121-90940948 | chr1: 90940755-90940774 | CUACCUACCUCUGCUUCAGU | − |
| 2230 | 84146 | ZNF644 | exon_07_c.1/ | chr1: 90940121-90940948 | chr1: 90940730-90940749 | GUGACAUUCAGAAUUCAGUA | − |
| 2231 | 84146 | ZNF644 | exon_07_c.1/ | chr1: 90940121-90940948 | chr1: 90940710-90940729 | GGGAGUAAUAUAAAGUCAGA | − |
| 2232 | 84146 | ZNF644 | exon_07_c.1/ | chr1: 90940121-90940948 | chr1: 90940687-90940706 | CACUUUAAUAAAUCAAGUAG | − |
| 2233 | 84146 | ZNF644 | exon_07_c.1/ | chr1: 90940121-90940948 | chr1: 90940630-90940649 | UGAUUGUGUCAAUACAGUAA | − |
| 2234 | 84146 | ZNF644 | exon_07_c.1/ | chr1: 90940121-90940948 | chr1: 90940617-90940636 | ACAGUAACGGGAAUUUCCUC | − |
| 2235 | 84146 | ZNF644 | exon_07_c.1/ | chr1: 90940121-90940948 | chr1: 90940601-90940620 | CCUCAGGUACAGAUGGAUUU | − |
| 2236 | 84146 | ZNF644 | exon_07_c.1/ | chr1: 90940121-90940948 | chr1: 90940285-90940304 | UGAUGAAGACUUAGAAUCUG | − |
| 2237 | 84146 | ZNF644 | exon_07_c.1/ | chr1: 90940121-90940948 | chr1: 90940245-90940264 | AUUUAUAACCCAGAUAAGUG | − |
| 2238 | 84146 | ZNF644 | exon_07_c.1/nc.2 | chr1: 90940070-90940120 | chr1: 90940102-90940121 | UCACAUUGCACUUUGUACAG | + |
| 2239 | 84146 | ZNF644 | exon_07_c.1/nc.2 | chr1: 90940070-90940120 | chr1: 90940101-90940120 | UUCACAUUGCACUUUGUACA | + |
| 2240 | 84146 | ZNF644 | exon_07_c.1/nc.2 | chr1: 90940070-90940120 | chr1: 90940060-90940079 | GAUACAUCAUAUGCCUGUGG | + |
| 2241 | 84146 | ZNF644 | exon_07_c.2/ | chr1: 90938272-90940069 | chr1: 90940052-90940071 | AUAUGAUGUAUCAUUUAGAU | − |
| 2242 | 84146 | ZNF644 | exon_07_c.2/ | chr1: 90938272-90940069 | chr1: 90940000-90940019 | CUCUACAAGCAUAUGGCCUU | + |
| 2243 | 84146 | ZNF644 | exon_07_c.2/ | chr1: 90938272-90940069 | chr1: 90939992-90940011 | AUGCUUGUAGAGAAUGUGGA | − |
| 2244 | 84146 | ZNF644 | exon_07_c.2/ | chr1: 90938272-90940069 | chr1: 90939811-90939830 | CAUGUUGCACAAAUGUUUUA | + |
| 2245 | 84146 | ZNF644 | exon_07_c.2/ | chr1: 90938272-90940069 | chr1: 90939810-90939829 | GCAUGUUGCACAAAUGUUUU | + |
| 2246 | 84146 | ZNF644 | exon_07_c.2/ | chr1: 90938272-90940069 | chr1: 90939782-90939801 | GUACCUUUUAUCUUUUUCAU | + |
| 2247 | 84146 | ZNF644 | exon_07_c.2/ | chr1: 90938272-90940069 | chr1: 90939711-90939730 | UGCCAUCGAGGCAUUGCACA | − |
| 2248 | 84146 | ZNF644 | exon_07_c.2/ | chr1: 90938272-90940069 | chr1: 90939670-90939689 | CAAUAUCAGAAGUGACCAUA | + |
| 2249 | 84146 | ZNF644 | exon_07_c.2/ | chr1: 90938272-90940069 | chr1: 90939624-90939643 | UUCAUGAAAGACUCUGUAGU | − |
| 2250 | 84146 | ZNF644 | exon_07_c.2/ | chr1: 90938272-90940069 | chr1: 90939578-90939597 | AGGACACAUCUUACAUAUGU | + |
| 2251 | 84146 | ZNF644 | exon_07_c.2/ | chr1: 90938272-90940069 | chr1: 90939504-90939523 | UCAUCAUGUGUUGAUUCAUU | − |
| 2252 | 84146 | ZNF644 | exon_07_c.2/ | chr1: 90938272-90940069 | chr1: 90939474-90939493 | UUUCUUUUAUCAAGUCCAAG | + |
| 2253 | 84146 | ZNF644 | exon_07_c.2/ | chr1: 90938272-90940069 | chr1: 90939329-90939348 | UGAAAAACUACUUGAUUGUG | + |
| 2254 | 84146 | ZNF644 | exon_07_c.2/ | chr1: 90938272-90940069 | chr1: 90939177-90939196 | GCUGCUUGAUGGAAAUACUU | + |
| 2255 | 84146 | ZNF644 | exon_07_c.2/ | chr1: 90938272-90940069 | chr1: 90939137-90939156 | CAAAUAGUGGCUAUUUGCCU | + |
| 2256 | 84146 | ZNF644 | exon_07_c.2/ | chr1: 90938272-90940069 | chr1: 90939113-90939132 | GACACAAAUAUGAAAACUAU | − |
| 2257 | 84146 | ZNF644 | exon_07_c.2/ | chr1: 90938272-90940069 | chr1: 90938951-90938970 | UUUGAAGCUUUCAGGCCUUU | + |
| 2258 | 84146 | ZNF644 | exon_07_c.2/ | chr1: 90938272-90940069 | chr1: 90938917-90938936 | AGCUGUAAAGAGAGUAAUUA | − |
| 2259 | 84146 | ZNF644 | exon_07_c.2/ | chr1: 90938272-90940069 | chr1: 90938774-90938793 | AAACAGAAGAUGAAAGUUCC | − |
| 2260 | 84146 | ZNF644 | exon_07_c.2/ | chr1: 90938272-90940069 | chr1: 90938753-90938772 | UCCUAACUCAACAUUAUCCC | + |
| 2261 | 84146 | ZNF644 | exon_07_c.2/ | chr1: 90938272-90940069 | chr1: 90938714-90938733 | ACUAUAGGUUUCAUCUUCUA | + |
| 2262 | 84146 | ZNF644 | exon_07_c.2/ | chr1: 90938272-90940069 | chr1: 90938699-90938718 | CUCUUGAUUAAUAUCACUAU | + |
| 2263 | 84146 | ZNF644 | exon_07_c.2/ | chr1: 90938272-90940069 | chr1: 90938649-90938668 | AAAGUGGAAGGUCAGGAGCC | − |
| 2264 | 84146 | ZNF644 | exon_07_c.2/ | chr1: 90938272-90940069 | chr1: 90938628-90938647 | CUAAGAGUAGCAUUUUCUCC | + |
| 2265 | 84146 | ZNF644 | exon_07_c.2/ | chr1: 90938272-90940069 | chr1: 90938436-90938455 | UCAAAUGUUGCUGGGCAGUA | + |
| 2266 | 84146 | ZNF644 | exon_07_c.2/ | chr1: 90938272-90940069 | chr1: 90938427-90938446 | ACACCUGUUUCAAAUGUUGC | + |
| 2267 | 84146 | ZNF644 | exon_07_c.2/ | chr1: 90938272-90940069 | chr1: 90938347-90938366 | GUUCUGGUGAUACAACAUGA | + |
| 2268 | 84146 | ZNF644 | exon_07_c.2/ | chr1: 90938272-90940069 | chr1: 90938307-90938326 | AUGCAGCAUUUCAAAAGAAC | − |
| 2269 | 84146 | ZNF644 | exon_07_c.2/ | chr1: 90938272-90940069 | chr1: 90938301-90938320 | CAUUUCAAAAGAACUGGCAC | − |
| 2270 | 84146 | ZNF644 | intron_07 | chr1: 90938091-90938271 | chr1: 90938248-90938267 | GUUCUCCAUGGGGAUGAUUU | |
| 2271 | 84146 | ZNF644 | intron_07 | chr1: 90938091-90938271 | chr1: 90938143-90938162 | AGAAUUAACUCAUUUUAGGG | − |
| 2272 | 84146 | ZNF644 | exon_08_c | chr1: 90937939-90938090 | chr1: 90938042-90938061 | CACACUUGUCAGCUCUGUGG | − |
| 2273 | 84146 | ZNF644 | exon_08_c | chr1: 90937939-90938090 | chr1: 90938000-90938019 | GGAUUAUCAAAUCAUGUUAG | − |
| 2274 | 84146 | ZNF644 | exon_08_c | chr1: 90937485-90937882 | chr1: 90937844-90937863 | UUUUGAGCUACAAAUGGUCU | + |
| 2275 | 84146 | ZNF644 | exon_08_c | chr1: 90937485-90937882 | chr1: 90937758-90937777 | CUGACAGAGCUUCAGUCUUU | + |
| 2276 | 84146 | ZNF644 | exon_08_c | chr1: 90937485-90937882 | chr1: 90937679-90937698 | UUUUUCCCACUGGGCAGUUC | + |
| 2277 | 84146 | ZNF644 | exon_08_c | chr1: 90937485-90937882 | chr1: 90937539-90937558 | AUGCGUUCUUCCAUUAAAUG | − |
| 2278 | 84146 | ZNF644 | exon_08_c | chr1: 90937485-90937882 | chr1: 90937506-90937525 | GAUGUAUCAGCCACAAAAAA | − |
| 2279 | 84146 | ZNF644 | exon_08_c | chr1: 90937485-90937882 | chr1: 90937487-90937506 | AUGGACUUGACUAUGCACUC | − |
| 2280 | 84146 | ZNF644 | intron_08 | chr1: 90935529-90937484 | chr1: 90937480-90937499 | UGACUAUGCACUCAGGUAAG | − |
| 2281 | 84146 | ZNF644 | intron_08 | chr1: 90935529-90937484 | chr1: 90937408-90937427 | CACAUAGAUUCCAUUAAAGA | + |
| 2282 | 84146 | ZNF644 | intron_08 | chr1: 90935529-90937484 | chr1: 90937165-90937184 | AUAAAACUUGCUCUGGCUGG | − |
| 2283 | 84146 | ZNF644 | intron_08 | chr1: 90935529-90937484 | chr1: 90937155-90937174 | CUCUGGCUGGAGGAUGGUUG | − |
| 2284 | 84146 | ZNF644 | intron_08 | chr1: 90935529-90937484 | chr1: 90937150-90937169 | GCUGGAGGAUGGUUGUGGAU | − |
| 2285 | 84146 | ZNF644 | intron_08 | chr1: 90935529-90937484 | chr1: 90937059-90937078 | UCUUGAAAACAUGAUCACCU | + |
| 2286 | 84146 | ZNF644 | intron_08 | chr1: 90935529-90937484 | chr1: 90937033-90937052 | UGUUGGUUUUUCCACUGCUU | − |
| 2287 | 84146 | ZNF644 | intron_08 | chr1: 90935529-90937484 | chr1: 90936940-90936959 | AUACAAUGAGGAAUUGGAGA | + |
| 2288 | 84146 | ZNF644 | intron_08 | chr1: 90935529-90937484 | chr1: 90936888-90936907 | GAACAUAAAACAUAUUUGUU | − |
| 2289 | 84146 | ZNF644 | intron_08 | chr1: 90935529-90937484 | chr1: 90936851-90936870 | AGAUAGUGCUCUAAUGGAAC | − |
| 2290 | 84146 | ZNF644 | intron_08 | chr1: 90935529-90937484 | chr1: 90936784-90936803 | UACAAAAUAUUCCUUUCAAA | + |
| 2291 | 84146 | ZNF644 | intron_08 | chr1: 90935529-90937484 | chr1: 90936592-90936611 | GUGUCAGAGGUAAUAGUUAA | − |
| 2292 | 84146 | ZNF644 | intron_08 | chr1: 90935529-90937484 | chr1: 90936542-90936561 | GUAAUGUUGUUUAUUUGCUU | − |
| 2293 | 84146 | ZNF644 | intron_08 | chr1: 90935529-90937484 | chr1: 90936540-90936559 | AAUGUUGUUUAUUUGCUUUG | − |
| 2294 | 84146 | ZNF644 | intron_08 | chr1: 90935529-90937484 | chr1: 90936467-90936486 | AGAUAACUUUGUAUUGUAAG | − |
| 2295 | 84146 | ZNF644 | intron_08 | chr1: 90935529-90937484 | chr1: 90936462-90936481 | ACUUUGUAUUGUAAGUGGUC | − |
| 2296 | 84146 | ZNF644 | intron_08 | chr1: 90935529-90937484 | chr1: 90936352-90936371 | UUUUCAGAAGGGUAAAACGC | − |
| 2297 | 84146 | ZNF644 | intron_08 | chr1: 90935529-90937484 | chr1: 90936351-90936370 | UUUCAGAAGGGUAAAACGCA | − |
| 2298 | 84146 | ZNF644 | intron_08 | chr1: 90935529-90937484 | chr1: 90936322-90936341 | UGUUAGCUGUGUUGACCAAG | − |
| 2299 | 84146 | ZNF644 | intron_08 | chr1: 90935529-90937484 | chr1: 90936303-90936322 | GGGGUAGUAUACUUCAGAGA | − |
| 2300 | 84146 | ZNF644 | intron_08 | chr1: 90935529-90937484 | chr1: 90936247-90936266 | AGAAACCAGUUGAAAUGUGU | − |
| 2301 | 84146 | ZNF644 | intron_08 | chr1: 90935529-90937484 | chr1: 90936246-90936265 | GAAACCAGUUGAAAUGUGUU | − |
| 2302 | 84146 | ZNF644 | intron_08 | chr1: 90935529-90937484 | chr1: 90936245-90936264 | AAACCAGUUGAAAUGUGUUG | − |
| 2303 | 84146 | ZNF644 | intron_08 | chr1: 90935529-90937484 | chr1: 90936244-90936263 | AACCAGUUGAAAUGUGUUGG | − |
| 2304 | 84146 | ZNF644 | intron_08 | chr1: 90935529-90937484 | chr1: 90936034-90936053 | UGUGUCUAACAGCUGUUCAG | − |
| 2305 | 84146 | ZNF644 | intron_08 | chr1: 90935529-90937484 | chr1: 90935983-90936002 | UAAAUUAGGCUUUCUGAUGA | + |
| 2306 | 84146 | ZNF644 | intron_08 | chr1: 90935529-90937484 | chr1: 90935969-90935988 | AAAAGUCUCAAUACUAAAUU | + |
| 2307 | 84146 | ZNF644 | intron_08 | chr1: 90935529-90937484 | chr1: 90935784-90935803 | AUCUGCAAGAAAGUAAUAAU | − |
| 2308 | 84146 | ZNF644 | intron_08 | chr1: 90935529-90937484 | chr1: 90935709-90935728 | UAGAGUCAACUCAUGCUCUU | + |
| 2309 | 84146 | ZNF644 | intron_08 | chr1: 90935529-90937484 | chr1: 90935648-90935667 | AGGUUAACUUAAAAUAAGGA | + |
| 2310 | 84146 | ZNF644 | intron_08 | chr1: 90935529-90937484 | chr1: 90935647-90935666 | UAGGUUAACUUAAAAUAAGG | + |
| 2311 | 84146 | ZNF644 | intron_08 | chr1: 90935529-90937484 | chr1: 90935559-90935578 | UCACUUUUUGAAGUGAGAAC | − |
| 2312 | 84146 | ZNF644 | exon_09_nc | chr1: 90935408-90935528 | chr1: 90935416-90935435 | AAGAGUGCUGGACUUUUGAC | − |
| 2313 | 84146 | ZNF644 | intron_09 | chr1: 90918392-90935407 | chr1: 90935195-90935214 | GCUCUUAUUGACUUAAUUUG | − |
| 2314 | 84146 | ZNF644 | intron_09 | chr1: 90918392-90935407 | chr1: 90935156-90935175 | CUCAAAGUUCCACACCUCUU | + |
| 2315 | 84146 | ZNF644 | intron_09 | chr1: 90918392-90935407 | chr1: 90935071-90935090 | UAUAAAGAGAGGUGUUUUAC | + |
| 2316 | 84146 | ZNF644 | intron_09 | chr1: 90918392-90935407 | chr1: 90934898-90934917 | AAUAUUGUAGUUCUAGCUUG | − |
| 2317 | 84146 | ZNF644 | intron_09 | chr1: 90918392-90935407 | chr1: 90934840-90934859 | UUCAAAAUGUAGACUUGAUU | − |
| 2318 | 84146 | ZNF644 | intron_09 | chr1: 90918392-90935407 | chr1: 90934787-90934806 | UCUAUAGAUUAGAUCUUUGA | − |
| 2319 | 84146 | ZNF644 | intron_09 | chr1: 90918392-90935407 | chr1: 90934786-90934805 | CUAUAGAUUAGAUCUUUGAA | − |
| 2320 | 84146 | ZNF644 | intron_09 | chr1: 90918392-90935407 | chr1: 90934785-90934804 | UAUAGAUUAGAUCUUUGAAG | − |
| 2321 | 84146 | ZNF644 | intron_09 | chr1: 90918392-90935407 | chr1: 90934755-90934774 | GUCAACACAAGCCAUUAUUA | + |
| 2322 | 84146 | ZNF644 | intron_09 | chr1: 90918392-90935407 | chr1: 90934605-90934624 | CACAGAUGAGCAGUAUUAAG | + |
| 2323 | 84146 | ZNF644 | intron_09 | chr1: 90918392-90935407 | chr1: 90934497-90934516 | AAAUGAGAUUUGCUGAAGCA | + |
| 2324 | 84146 | ZNF644 | intron_09 | chr1: 90918392-90935407 | chr1: 90934474-90934493 | GGGUAUAUUUUAAGAUGUGA | + |
| 2325 | 84146 | ZNF644 | intron_09 | chr1: 90918392-90935407 | chr1: 90934454-90934473 | GUAGAUCUAUGUCAAAAAGA | + |
| 2326 | 84146 | ZNF644 | intron_09 | chr1: 90918392-90935407 | chr1: 90934453-90934472 | UGUAGAUCUAUGUCAAAAAG | + |
| 2327 | 84146 | ZNF644 | intron_09 | chr1: 90918392-90935407 | chr1: 90934301-90934320 | UUUUGAAAGCAAGAACAUAU | + |
| 2328 | 84146 | ZNF644 | intron_09 | chr1: 90918392-90935407 | chr1: 90934187-90934206 | AUAUUAAAAAGAGAGGAGUG | − |
| 2329 | 84146 | ZNF644 | intron_09 | chr1: 90918392-90935407 | chr1: 90934137-90934156 | AAUUAUAAUCUCAGAGUAGG | + |
| 2330 | 84146 | ZNF644 | intron_09 | chr1: 90918392-90935407 | chr1: 90934106-90934125 | GCCAAGCCUGAUAAAGGUGA | + |
| 2331 | 84146 | ZNF644 | intron_09 | chr1: 90918392-90935407 | chr1: 90934105-90934124 | UGCCAAGCCUGAUAAAGGUG | + |
| 2332 | 84146 | ZNF644 | intron_09 | chr1: 90918392-90935407 | chr1: 90934100-90934119 | UCACUUGCCAAGCCUGAUAA | + |
| 2333 | 84146 | ZNF644 | intron_09 | chr1: 90918392-90935407 | chr1: 90934087-90934106 | CAAGUGAUUGCAGACAUUAG | − |
| 2334 | 84146 | ZNF644 | intron_09 | chr1: 90918392-90935407 | chr1: 90934084-90934103 | GUGAUUGCAGACAUUAGAGG | − |
| 2335 | 84146 | ZNF644 | intron_09 | chr1: 90918392-90935407 | chr1: 90934051-90934070 | AAAUAAGAGACUAUACAAGU | − |
| 2336 | 84146 | ZNF644 | intron_09 | chr1: 90918392-90935407 | chr1: 90933699-90933718 | AAUGGAAAAUUCUAAAAUGA | − |
| 2337 | 84146 | ZNF644 | intron_09 | chr1: 90918392-90935407 | chr1: 90933364-90933383 | UUUAAACCCACCUUCAGGCU | + |
| 2338 | 84146 | ZNF644 | intron_09 | chr1: 90918392-90935407 | chr1: 90933292-90933311 | CAGAGAUCCAAGAAGUGCUU | − |
| 2339 | 84146 | ZNF644 | intron_09 | chr1: 90918392-90935407 | chr1: 90933219-90933238 | GAUUAUGCAAAUAUCCAAUA | − |
| 2340 | 84146 | ZNF644 | intron_09 | chr1: 90918392-90935407 | chr1: 90933074-90933093 | AAUCUUCAUAAGUGCAUUCU | + |
| 2341 | 84146 | ZNF644 | intron_09 | chr1: 90918392-90935407 | chr1: 90933036-90933055 | AUCAGAGAGAAAAGUAAUGU | − |
| 2342 | 84146 | ZNF644 | intron_09 | chr1: 90918392-90935407 | chr1: 90933028-90933047 | GAAAAGUAAUGUAGGAUGAC | − |
| 2343 | 84146 | ZNF644 | intron_09 | chr1: 90918392-90935407 | chr1: 90933015-90933034 | GGAUGACUGGGAAUAUGUUU | − |
| 2344 | 84146 | ZNF644 | intron_09 | chr1: 90918392-90935407 | chr1: 90932996-90933015 | UAGGUCCUGAAGAAUAAAAG | − |
| 2345 | 84146 | ZNF644 | intron_09 | chr1: 90918392-90935407 | chr1: 90932995-90933014 | AGGUCCUGAAGAAUAAAAGA | − |
| 2346 | 84146 | ZNF644 | intron_09 | chr1: 90918392-90935407 | chr1: 90932940-90932959 | UCCUAGUACUUCUAAUAUUU | + |
| 2347 | 84146 | ZNF644 | intron_09 | chr1: 90918392-90935407 | chr1: 90932923-90932942 | GGAAAUAGAAAGCGUGUGUU | − |
| 2348 | 84146 | ZNF644 | intron_09 | chr1: 90918392-90935407 | chr1: 90932902-90932921 | GGCAGUCCUAGGCAAAGAAA | − |
| 2349 | 84146 | ZNF644 | intron_09 | chr1: 90918392-90935407 | chr1: 90932809-90932828 | AAACUAAUGCUUCUUUUUCU | − |
| 2350 | 84146 | ZNF644 | intron_09 | chr1: 90918392-90935407 | chr1: 90932655-90932674 | ACAAUACAGAAUUCUCUAUC | + |
| 2351 | 84146 | ZNF644 | intron_09 | chr1: 90918392-90935407 | chr1: 90932605-90932624 | AUAACAGUGAUAUUGAUAUG | + |
| 2352 | 84146 | ZNF644 | intron_09 | chr1: 90918392-90935407 | chr1: 90932582-90932601 | GCAGUGUUCCUAAAAACCUA | − |
| 2353 | 84146 | ZNF644 | intron_09 | chr1: 90918392-90935407 | chr1: 90932572-90932591 | UAAAAACCUAUGGCCCUAGA | − |
| 2354 | 84146 | ZNF644 | intron_09 | chr1: 90918392-90935407 | chr1: 90932556-90932575 | UAACCAAGUGGAACCUUCUA | + |
| 2355 | 84146 | ZNF644 | intron_09 | chr1: 90918392-90935407 | chr1: 90932555-90932574 | AUAACCAAGUGGAACCUUCU | + |
| 2356 | 84146 | ZNF644 | intron_09 | chr1: 90918392-90935407 | chr1: 90932422-90932441 | CAAAAUUGGAACAAUGUAAA | − |
| 2357 | 84146 | ZNF644 | intron_09 | chr1: 90918392-90935407 | chr1: 90932373-90932392 | UACACAUACUGGACAUGGUU | + |
| 2358 | 84146 | ZNF644 | intron_09 | chr1: 90918392-90935407 | chr1: 90932368-90932387 | AGAAAUACACAUACUGGACA | + |
| 2359 | 84146 | ZNF644 | intron_09 | chr1: 90918392-90935407 | chr1: 90932254-90932273 | UAUCACAGGUAACCAAAAGC | + |
| 2360 | 84146 | ZNF644 | intron_09 | chr1: 90918392-90935407 | chr1: 90932216-90932235 | AGGAGCUGUGGCUUCUAAGA | + |
| 2361 | 84146 | ZNF644 | intron_09 | chr1: 90918392-90935407 | chr1: 90932217-90932236 | UUCUUAGAAGCCACAGCUCC | − |
| 2362 | 84146 | ZNF644 | intron_09 | chr1: 90918392-90935407 | chr1: 90932118-90932137 | AUAUCCUUUUCAGUGUAUUC | + |
| 2363 | 84146 | ZNF644 | intron_09 | chr1: 90918392-90935407 | chr1: 90932125-90932144 | GUAUCCUGAAUACACUGAAA | − |
| 2364 | 84146 | ZNF644 | intron_09 | chr1: 90918392-90935407 | chr1: 90932085-90932104 | UACCAGUAAACAGGUAUUUU | + |
| 2365 | 84146 | ZNF644 | intron_09 | chr1: 90918392-90935407 | chr1: 90932064-90932083 | CUUAAAUACUUCUAAUGUGA | − |
| 2366 | 84146 | ZNF644 | intron_09 | chr1: 90918392-90935407 | chr1: 90931976-90931995 | ACAAAUAAGUGCAUGUUUCU | + |
| 2367 | 84146 | ZNF644 | intron_09 | chr1: 90918392-90935407 | chr1: 90931854-90931873 | AGGAAAGUAGAAACAGAACU | − |
| 2368 | 84146 | ZNF644 | intron_09 | chr1: 90918392-90935407 | chr1: 90931767-90931786 | CAUAAUAGCAAAACUAAUUG | − |
| 2369 | 84146 | ZNF644 | intron_09 | chr1: 90918392-90935407 | chr1: 90931766-90931785 | AUAAUAGCAAAACUAAUUGU | − |
| 2370 | 84146 | ZNF644 | intron_09 | chr1: 90918392-90935407 | chr1: 90931729-90931748 | GGUUCCUUUUACAAAUAUUA | + |
| 2371 | 84146 | ZNF644 | intron_09 | chr1: 90918392-90935407 | chr1: 90931725-90931744 | AUUUGUAAAAGGAACCGAUG | − |
| 2372 | 84146 | ZNF644 | intron_09 | chr1: 90918392-90935407 | chr1: 90931684-90931703 | AAUUGAAGAUUUGGAAGAGG | − |
| 2373 | 84146 | ZNF644 | intron_09 | chr1: 90918392-90935407 | chr1: 90931673-90931692 | UGGAAGAGGUGGGCAGACUU | − |
| 2374 | 84146 | ZNF644 | intron_09 | chr1: 90918392-90935407 | chr1: 90931600-90931619 | AAGAUAAACUGGGACAGUUC | + |
| 2375 | 84146 | ZNF644 | intron_09 | chr1: 90918392-90935407 | chr1: 90931589-90931608 | CUACCCAACAGAAGAUAAAC | + |
| 2376 | 84146 | ZNF644 | intron_09 | chr1: 90918392-90935407 | chr1: 90931440-90931459 | CUGCCUUAAAUUUACAAUAU | + |
| 2377 | 84146 | ZNF644 | intron_09 | chr1: 90918392-90935407 | chr1: 90931446-90931465 | UAUCCUAUAUUGUAAAUUUA | − |
| 2378 | 84146 | ZNF644 | intron_09 | chr1: 90918392-90935407 | chr1: 90931394-90931413 | UUAUUUAUGGAAUGCAGUAA | − |
| 2379 | 84146 | ZNF644 | intron_09 | chr1: 90918392-90935407 | chr1: 90931390-90931409 | UUAUGGAAUGCAGUAAUGGU | − |
| 2380 | 84146 | ZNF644 | intron_09 | chr1: 90918392-90935407 | chr1: 90931323-90931342 | UGCUCAUACAGAUUUUUUUC | − |
| 2381 | 84146 | ZNF644 | intron_09 | chr1: 90918392-90935407 | chr1: 90931234-90931253 | UAGCUUAUAUUAAUUACUUC | − |
| 2382 | 84146 | ZNF644 | intron_09 | chr1: 90918392-90935407 | chr1: 90931139-90931158 | AUAGAACUAUGUUCUUAAUA | + |
| 2383 | 84146 | ZNF644 | intron_09 | chr1: 90918392-90935407 | chr1: 90931128-90931147 | UAGUUCUAUUAAGUUAUUAA | − |
| 2384 | 84146 | ZNF644 | intron_09 | chr1: 90918392-90935407 | chr1: 90931067-90931086 | UAAAAGAGGCACUAGUACUU | + |
| 2385 | 84146 | ZNF644 | intron_09 | chr1: 90918392-90935407 | chr1: 90931031-90931050 | AUUAAUUUAUCAUUCAAUCC | + |
| 2386 | 84146 | ZNF644 | intron_09 | chr1: 90918392-90935407 | chr1: 90930577-90930596 | UAUGAUAGUGACUUCUUAAG | + |
| 2387 | 84146 | ZNF644 | intron_09 | chr1: 90918392-90935407 | chr1: 90930539-90930558 | CCUAAGAGAAAGAGGCAGUU | + |
| 2388 | 84146 | ZNF644 | intron_09 | chr1: 90918392-90935407 | chr1: 90930542-90930561 | CCUAACUGCCUCUUUCUCUU | − |
| 2389 | 84146 | ZNF644 | intron_09 | chr1: 90918392-90935407 | chr1: 90930482-90930501 | CAUAACUCUUCUGUGGCAGC | − |
| 2390 | 84146 | ZNF644 | intron_09 | chr1: 90918392-90935407 | chr1: 90930408-90930427 | UAAAUCAAAUGCUAACCUCU | − |
| 2391 | 84146 | ZNF644 | intron_09 | chr1: 90918392-90935407 | chr1: 90929984-90930003 | ACGUAUCAAGAAAGUGUUCU | + |
| 2392 | 84146 | ZNF644 | intron_09 | chr1: 90918392-90935407 | chr1: 90929777-90929796 | UUCAGCUUUAAUGAUAUAAU | − |
| 2393 | 84146 | ZNF644 | intron_09 | chr1: 90918392-90935407 | chr1: 90929751-90929770 | GUACUUGUUACAGUCCUGUG | + |
| 2394 | 84146 | ZNF644 | intron_09 | chr1: 90918392-90935407 | chr1: 90929717-90929736 | CACAGUAUCAAGUGCUGAAU | − |
| 2395 | 84146 | ZNF644 | intron_09 | chr1: 90918392-90935407 | chr1: 90929680-90929699 | GAAAAGUCAUUCUUAUGUUG | − |
| 2396 | 84146 | ZNF644 | intron_09 | chr1: 90918392-90935407 | chr1: 90929597-90929616 | ACAAAUAAUUUAAUGCACAU | + |
| 2397 | 84146 | ZNF644 | intron_09 | chr1: 90918392-90935407 | chr1: 90929435-90929454 | CAGAAGCAAGUUUGCUCAUU | + |
| 2398 | 84146 | ZNF644 | intron_09 | chr1: 90918392-90935407 | chr1: 90929385-90929404 | AUACCCUCCUCAGCCCAGUC | + |
| 2399 | 84146 | ZNF644 | intron_09 | chr1: 90918392-90935407 | chr1: 90929274-90929293 | CAAGAUAGUAUACAACUAAA | − |
| 2400 | 84146 | ZNF644 | intron_09 | chr1: 90918392-90935407 | chr1: 90929250-90929269 | AUAUUGGCUUUAAGUGUUGU | − |
| 2401 | 84146 | ZNF644 | intron_09 | chr1: 90918392-90935407 | chr1: 90929242-90929261 | UUUAAGUGUUGUAGGAGUUC | − |
| 2402 | 84146 | ZNF644 | intron_09 | chr1: 90918392-90935407 | chr1: 90929194-90929213 | AAUUCCCAGCUGCCCUCAAA | + |
| 2403 | 84146 | ZNF644 | intron_09 | chr1: 90918392-90935407 | chr1: 90929201-90929220 | GCCUCCUUUUGAGGGCAGCU | − |
| 2404 | 84146 | ZNF644 | intron_09 | chr1: 90918392-90935407 | chr1: 90929149-90929168 | UUCAAUGAGACAAACAGUAA | + |
| 2405 | 84146 | ZNF644 | intron_09 | chr1: 90918392-90935407 | chr1: 90929032-90929051 | AUAAAAUUUUGUAUGGCUGC | + |
| 2406 | 84146 | ZNF644 | intron_09 | chr1: 90918392-90935407 | chr1: 90928941-90928960 | UUAUAUUAUGUUUAUGCAGC | + |
| 2407 | 84146 | ZNF644 | intron_09 | chr1: 90918392-90935407 | chr1: 90928936-90928955 | AUAAACAUAAUAUAACCCAG | − |
| 2408 | 84146 | ZNF644 | intron_09 | chr1: 90918392-90935407 | chr1: 90928918-90928937 | AUACUUGAUCUUUUCCCACU | + |
| 2409 | 84146 | ZNF644 | intron_09 | chr1: 90918392-90935407 | chr1: 90928865-90928884 | CAUCCUUCAACUUCAGGGUC | + |
| 2410 | 84146 | ZNF644 | intron_09 | chr1: 90918392-90935407 | chr1: 90928871-90928890 | CAGCCAGACCCUGAAGUUGA | − |
| 2411 | 84146 | ZNF644 | intron_09 | chr1: 90918392-90935407 | chr1: 90928794-90928813 | AGAACAUAGUGUGGACACAA | − |
| 2412 | 84146 | ZNF644 | intron_09 | chr1: 90918392-90935407 | chr1: 90928782-90928801 | GGACACAAAGGGAUGAAAUU | − |
| 2413 | 84146 | ZNF644 | intron_09 | chr1: 90918392-90935407 | chr1: 90928693-90928712 | AAAUUGUAGUGCCAGAAAGG | − |
| 2414 | 84146 | ZNF644 | intron_09 | chr1: 90918392-90935407 | chr1: 90928687-90928706 | UAGUGCCAGAAAGGUGGGGU | − |
| 2415 | 84146 | ZNF644 | intron_09 | chr1: 90918392-90935407 | chr1: 90928671-90928690 | GGGUUGGUGCUUUGAAUGCU | − |
| 2416 | 84146 | ZNF644 | intron_09 | chr1: 90918392-90935407 | chr1: 90928644-90928663 | AAAAAUUAGGAAGUCAUUAA | − |
| 2417 | 84146 | ZNF644 | intron_09 | chr1: 90918392-90935407 | chr1: 90927534-90927553 | AUCAAGUUAUUAACAGUUAA | + |
| 2418 | 84146 | ZNF644 | intron_09 | chr1: 90918392-90935407 | chr1: 90927502-90927521 | UGGAGCAAUAACCACCAAAA | + |
| 2419 | 84146 | ZNF644 | intron_09 | chr1: 90918392-90935407 | chr1: 90927128-90927147 | AUGCUAAUUCCAAAAGCCUU | + |
| 2420 | 84146 | ZNF644 | intron_09 | chr1: 90918392-90935407 | chr1: 90927096-90927115 | UUUCAGUUAGCUAUACAUCA | + |
| 2421 | 84146 | ZNF644 | intron_09 | chr1: 90918392-90935407 | chr1: 90926915-90926934 | GUUGGAAUUAACUUUGUAGU | − |
| 2422 | 84146 | ZNF644 | intron_09 | chr1: 90918392-90935407 | chr1: 90926785-90926804 | UAAUGUCACUGACCUGGUAC | + |
| 2423 | 84146 | ZNF644 | intron_09 | chr1: 90918392-90935407 | chr1: 90926773-90926792 | UGACAUUAAGAAUGUAAAGG | − |
| 2424 | 84146 | ZNF644 | intron_09 | chr1: 90918392-90935407 | chr1: 90926626-90926645 | CAAUCUGCUACCAUGCUGUC | − |
| 2425 | 84146 | ZNF644 | intron_09 | chr1: 90918392-90935407 | chr1: 90926619-90926638 | CUACCAUGCUGUCUGGUAGA | − |
| 2426 | 84146 | ZNF644 | intron_09 | chr1: 90918392-90935407 | chr1: 90926520-90926539 | UUGUAGAUCUCCUGCUUUUA | + |
| 2427 | 84146 | ZNF644 | intron_09 | chr1: 90918392-90935407 | chr1: 90926533-90926552 | CAAAGUAUCACCUUAAAAGC | − |
| 2428 | 84146 | ZNF644 | intron_09 | chr1: 90918392-90935407 | chr1: 90926284-90926303 | AACAUGGGAUGUAUGUAACA | + |
| 2429 | 84146 | ZNF644 | intron_09 | chr1: 90918392-90935407 | chr1: 90926158-90926177 | GAUCACCAACUGGUUCUAGC | + |
| 2430 | 84146 | ZNF644 | intron_09 | chr1: 90918392-90935407 | chr1: 90925731-90925750 | GCUUGUUUUUUGCUGCUCAU | − |
| 2431 | 84146 | ZNF644 | intron_09 | chr1: 90918392-90935407 | chr1: 90925717-90925736 | GCUCAUUGGUUUCAUAAGGA | − |
| 2432 | 84146 | ZNF644 | intron_09 | chr1: 90918392-90935407 | chr1: 90925186-90925205 | GACUCUCAUGCCCCCCACUG | + |
| 2433 | 84146 | ZNF644 | intron_09 | chr1: 90918392-90935407 | chr1: 90925081-90925100 | AGAAAUUCAUGUUAGAGAGA | + |
| 2434 | 84146 | ZNF644 | intron_09 | chr1: 90918392-90935407 | chr1: 90925075-90925094 | UAACAUGAAUUUCUUGAAUG | − |
| 2435 | 84146 | ZNF644 | intron_09 | chr1: 90918392-90935407 | chr1: 90925021-90925040 | CAUGCCUUGUUAUAUUAUAA | + |
| 2436 | 84146 | ZNF644 | intron_09 | chr1: 90918392-90935407 | chr1: 90925028-90925047 | UUAGCCCUUAUAAUAUAACA | − |
| 2437 | 84146 | ZNF644 | intron_09 | chr1: 90918392-90935407 | chr1: 90924999-90925018 | GUUGGCCAGAGCUAAAUUUU | − |
| 2438 | 84146 | ZNF644 | intron_09 | chr1: 90918392-90935407 | chr1: 90924976-90924995 | UAAUGUUUUAAUUGACUUUA | − |
| 2439 | 84146 | ZNF644 | intron_09 | chr1: 90918392-90935407 | chr1: 90924933-90924952 | GACUUUCUCUAUUGGAGGUU | − |
| 2440 | 84146 | ZNF644 | intron_09 | chr1: 90918392-90935407 | chr1: 90924932-90924951 | ACUUUCUCUAUUGGAGGUUA | − |
| 2441 | 84146 | ZNF644 | intron_09 | chr1: 90918392-90935407 | chr1: 90924909-90924928 | CAAAAGGAUGGCUUAUUGGU | − |
| 2442 | 84146 | ZNF644 | intron_09 | chr1: 90918392-90935407 | chr1: 90924878-90924897 | UUCUACUUCUGCAGUAAGUG | + |
| 2443 | 84146 | ZNF644 | intron_09 | chr1: 90918392-90935407 | chr1: 90924816-90924835 | AACCUCAUUCUUCAAACCCU | + |
| 2444 | 84146 | ZNF644 | intron_09 | chr1: 90918392-90935407 | chr1: 90924766-90924785 | AUUGUAGCUUAGCUGGAAGU | − |
| 2445 | 84146 | ZNF644 | intron_09 | chr1: 90918392-90935407 | chr1: 90924692-90924711 | UGGCUGGAAUUACACGUGUA | − |
| 2446 | 84146 | ZNF644 | intron_09 | chr1: 90918392-90935407 | chr1: 90924446-90924465 | UACGGAGAUUUUAGAGGGCA | − |
| 2447 | 84146 | ZNF644 | intron_09 | chr1: 90918392-90935407 | chr1: 90924341-90924360 | UCAGAUGUGUCUUACAAAAG | − |
| 2448 | 84146 | ZNF644 | intron_09 | chr1: 90918392-90935407 | chr1: 90924224-90924243 | UAUUACUUCUUUGUGGUGAC | + |
| 2449 | 84146 | ZNF644 | intron_09 | chr1: 90918392-90935407 | chr1: 90924152-90924171 | AUUGUUAGCUUUUUUAGCUU | − |
| 2450 | 84146 | ZNF644 | intron_09 | chr1: 90918392-90935407 | chr1: 90924010-90924029 | GUAGAGGGGAUGAUUACUAA | + |
| 2451 | 84146 | ZNF644 | intron_09 | chr1: 90918392-90935407 | chr1: 90923942-90923961 | UCCAUUCAAGUUUAAAUAAA | − |
| 2452 | 84146 | ZNF644 | intron_09 | chr1: 90918392-90935407 | chr1: 90923904-90923923 | UAGUUCAGAGAAAGAGUUAA | + |
| 2453 | 84146 | ZNF644 | intron_09 | chr1: 90918392-90935407 | chr1: 90923902-90923921 | AACUCUUUCUCUGAACUAUA | − |
| 2454 | 84146 | ZNF644 | intron_09 | chr1: 90918392-90935407 | chr1: 90923864-90923883 | AAUUUAACUUCCUACUUUGU | − |
| 2455 | 84146 | ZNF644 | intron_09 | chr1: 90918392-90935407 | chr1: 90923837-90923856 | ACUCAAGCAACCAGUCCUUG | − |
| 2456 | 84146 | ZNF644 | intron_09 | chr1: 90918392-90935407 | chr1: 90923778-90923797 | GCAAAGGAAAUCUGCUUUCC | − |
| 2457 | 84146 | ZNF644 | intron_09 | chr1: 90918392-90935407 | chr1: 90923752-90923771 | UUUGCAUUUCAUCCUCUUCC | − |
| 2458 | 84146 | ZNF644 | intron_09 | chr1: 90918392-90935407 | chr1: 90923731-90923750 | GGUAAAUUAUUAAAACAAGC | − |
| 2459 | 84146 | ZNF644 | intron_09 | chr1: 90918392-90935407 | chr1: 90923690-90923709 | CUCAAUCACACAGCAAUCAG | + |
| 2460 | 84146 | ZNF644 | intron_09 | chr1: 90918392-90935407 | chr1: 90923477-90923496 | AUAAUGUAAACUACUCAUCU | − |
| 2461 | 84146 | ZNF644 | intron_09 | chr1: 90918392-90935407 | chr1: 90923453-90923472 | AAUAAUGAAGUGGUAUAGCU | − |
| 2462 | 84146 | ZNF644 | intron_09 | chr1: 90918392-90935407 | chr1: 90923424-90923443 | GAACUGAUGUUGAUAGGAGU | − |
| 2463 | 84146 | ZNF644 | intron_09 | chr1: 90918392-90935407 | chr1: 90923422-90923441 | ACUGAUGUUGAUAGGAGUAG | − |
| 2464 | 84146 | ZNF644 | intron_09 | chr1: 90918392-90935407 | chr1: 90923323-90923342 | UGGGCCGAGAGAUUGAGGGG | − |
| 2465 | 84146 | ZNF644 | intron_09 | chr1: 90918392-90935407 | chr1: 90923273-90923292 | AUAGUAGAUACUCAGGUCCC | − |
| 2466 | 84146 | ZNF644 | intron_09 | chr1: 90918392-90935407 | chr1: 90923230-90923249 | CCACAUAACUCAUUUUGCAU | + |
| 2467 | 84146 | ZNF644 | intron_09 | chr1: 90918392-90935407 | chr1: 90922956-90922975 | CCUCAAAUGAAAAAGAAUCA | + |
| 2468 | 84146 | ZNF644 | intron_09 | chr1: 90918392-90935407 | chr1: 90922955-90922974 | GCCUCAAAUGAAAAAGAAUC | + |
| 2469 | 84146 | ZNF644 | intron_09 | chr1: 90918392-90935407 | chr1: 90922959-90922978 | CCCUGAUUCUUUUUCAUUUG | − |
| 2470 | 84146 | ZNF644 | intron_09 | chr1: 90918392-90935407 | chr1: 90922914-90922933 | UGCAGUCUUUUUAUAUGCUU | − |
| 2471 | 84146 | ZNF644 | intron_09 | chr1: 90918392-90935407 | chr1: 90922879-90922898 | CUGUUGUCACUAAUCCUUCA | + |
| 2472 | 84146 | ZNF644 | intron_09 | chr1: 90918392-90935407 | chr1: 90922640-90922659 | AGGUAAAUUGUCACGGGAGU | + |
| 2473 | 84146 | ZNF644 | intron_09 | chr1: 90918392-90935407 | chr1: 90922550-90922569 | UCUUACACUUGUGUAUCCUU | − |
| 2474 | 84146 | ZNF644 | intron_09 | chr1: 90918392-90935407 | chr1: 90922503-90922522 | AGGUCGUGCUGGCAGUGGUG | + |
| 2475 | 84146 | ZNF644 | intron_09 | chr1: 90918392-90935407 | chr1: 90922480-90922499 | CACUAUUAAAAAGUUGGUGC | + |
| 2476 | 84146 | ZNF644 | intron_09 | chr1: 90918392-90935407 | chr1: 90922474-90922493 | UUCUAUCACUAUUAAAAAGU | + |
| 2477 | 84146 | ZNF644 | intron_09 | chr1: 90918392-90935407 | chr1: 90922420-90922439 | AGCUCAUUCAGAACUACAGU | + |
| 2478 | 84146 | ZNF644 | intron_09 | chr1: 90918392-90935407 | chr1: 90922389-90922408 | UUGUCCACCAAACUUAACAC | − |
| 2479 | 84146 | ZNF644 | intron_09 | chr1: 90918392-90935407 | chr1: 90922336-90922355 | CACAGUAUACCCCUACGAGA | + |
| 2480 | 84146 | ZNF644 | intron_09 | chr1: 90918392-90935407 | chr1: 90922290-90922309 | UAGGACAAAGUGAAACAACU | + |
| 2481 | 84146 | ZNF644 | intron_09 | chr1: 90918392-90935407 | chr1: 90922271-90922290 | AAUUGGUUGCUGGUAUCUCU | + |
| 2482 | 84146 | ZNF644 | intron_09 | chr1: 90918392-90935407 | chr1: 90922235-90922254 | AAACAAGACUUUUAAGUUAU | − |
| 2483 | 84146 | ZNF644 | intron_09 | chr1: 90918392-90935407 | chr1: 90922234-90922253 | AACAAGACUUUUAAGUUAUU | − |
| 2484 | 84146 | ZNF644 | intron_09 | chr1: 90918392-90935407 | chr1: 90922060-90922079 | AAGCAAUUAUUGUAUAAAAU | − |
| 2485 | 84146 | ZNF644 | intron_09 | chr1: 90918392-90935407 | chr1: 90921764-90921783 | AAGACAAAUAUCUUACUGAA | − |
| 2486 | 84146 | ZNF644 | intron_09 | chr1: 90918392-90935407 | chr1: 90921688-90921707 | CUGAGAAUAUUUUACUCAUA | + |
| 2487 | 84146 | ZNF644 | intron_09 | chr1: 90918392-90935407 | chr1: 90921637-90921656 | CUUGAAAGAUAACAGUCUUU | − |
| 2488 | 84146 | ZNF644 | intron_09 | chr1: 90918392-90935407 | chr1: 90921578-90921597 | AUAGUAAUCUUAGGAAGGUU | − |
| 2489 | 84146 | ZNF644 | intron_09 | chr1: 90918392-90935407 | chr1: 90921570-90921589 | CUUAGGAAGGUUUGGAUGAC | − |
| 2490 | 84146 | ZNF644 | intron_09 | chr1: 90918392-90935407 | chr1: 90921438-90921457 | UGUAAAGAGAAAAGAGUGUG | − |
| 2491 | 84146 | ZNF644 | intron_09 | chr1: 90918392-90935407 | chr1: 90921355-90921374 | ACAAAGCCUUUUUUUGUAAU | + |
| 2492 | 84146 | ZNF644 | intron_09 | chr1: 90918392-90935407 | chr1: 90921224-90921243 | AUCUGUAUUUAAUGUUCUAG | − |
| 2493 | 84146 | ZNF644 | intron_09 | chr1: 90918392-90935407 | chr1: 90921217-90921236 | UUUAAUGUUCUAGUGGCUCC | − |
| 2494 | 84146 | ZNF644 | intron_09 | chr1: 90918392-90935407 | chr1: 90921185-90921204 | AGGGAAAAGUAGUUUUGUUC | + |
| 2495 | 84146 | ZNF644 | intron_09 | chr1: 90918392-90935407 | chr1: 90921165-90921184 | AGAGAUUUUAUUAUAUCUAG | + |
| 2496 | 84146 | ZNF644 | intron_09 | chr1: 90918392-90935407 | chr1: 90921152-90921171 | AAUCUCUUAAGUGAGAAUAU | − |
| 2497 | 84146 | ZNF644 | intron_09 | chr1: 90918392-90935407 | chr1: 90921077-90921096 | UAAAUCAUCCUUUUAUGUAG | − |
| 2498 | 84146 | ZNF644 | intron_09 | chr1: 90918392-90935407 | chr1: 90921034-90921053 | CAUAUUAAAACUGCACAAAA | + |
| 2499 | 84146 | ZNF644 | intron_09 | chr1: 90918392-90935407 | chr1: 90920996-90921015 | ACAGAUUUUCCCAAAGGUAU | − |
| 2500 | 84146 | ZNF644 | intron_09 | chr1: 90918392-90935407 | chr1: 90920984-90921003 | AAAGGUAUGGGCAGUAUCAC | − |
| 2501 | 84146 | ZNF644 | intron_09 | chr1: 90918392-90935407 | chr1: 90920979-90920998 | UAUGGGCAGUAUCACAGGAG | − |
| 2502 | 84146 | ZNF644 | intron_09 | chr1: 90918392-90935407 | chr1: 90920966-90920985 | ACAGGAGUGGGUUUGGAUAU | − |
| 2503 | 84146 | ZNF644 | intron_09 | chr1: 90918392-90935407 | chr1: 90920903-90920922 | GCUCAAAAAUAUAACAUUUC | − |
| 2504 | 84146 | ZNF644 | intron_09 | chr1: 90918392-90935407 | chr1: 90920793-90920812 | AACUUUGUGAAUAAGUUUCU | − |
| 2505 | 84146 | ZNF644 | intron_09 | chr1: 90918392-90935407 | chr1: 90920746-90920765 | AACCUUAACUUUGGAGCCCU | − |
| 2506 | 84146 | ZNF644 | intron_09 | chr1: 90918392-90935407 | chr1: 90920727-90920746 | UCAAUCAUCUCUAGAACCCA | + |
| 2507 | 84146 | ZNF644 | intron_09 | chr1: 90918392-90935407 | chr1: 90920680-90920699 | GUAUUAUAACCUUGGAAAGC | + |
| 2508 | 84146 | ZNF644 | intron_09 | chr1: 90918392-90935407 | chr1: 90920658-90920677 | UUGUAGCAAUGAGUAUUUGA | + |
| 2509 | 84146 | ZNF644 | intron_09 | chr1: 90918392-90935407 | chr1: 90920371-90920390 | UAGGGGUGUAAAGCAAAUUG | + |
| 2510 | 84146 | ZNF644 | intron_09 | chr1: 90918392-90935407 | chr1: 90920352-90920371 | GUUCACAGAAUGUUAAUGUU | + |
| 2511 | 84146 | ZNF644 | intron_09 | chr1: 90918392-90935407 | chr1: 90920163-90920182 | GAUGGAUCCUUGUCUGUGUU | + |
| 2512 | 84146 | ZNF644 | intron_09 | chr1: 90918392-90935407 | chr1: 90920052-90920071 | UGAGCAAUCUGAAUUUAAGU | + |
| 2513 | 84146 | ZNF644 | intron_09 | chr1: 90918392-90935407 | chr1: 90920051-90920070 | GUGAGCAAUCUGAAUUUAAG | + |
| 2514 | 84146 | ZNF644 | intron_09 | chr1: 90918392-90935407 | chr1: 90920029-90920048 | AACCUUUUUCCUAAUCGCUU | + |
| 2515 | 84146 | ZNF644 | intron_09 | chr1: 90918392-90935407 | chr1: 90920020-90920039 | GGAAAAAGGUUUUUCCUACU | − |
| 2516 | 84146 | ZNF644 | intron_09 | chr1: 90918392-90935407 | chr1: 90919847-90919866 | GAAUAUUACCUGUAUUAAAU | − |
| 2517 | 84146 | ZNF644 | intron_09 | chr1: 90918392-90935407 | chr1: 90919667-90919686 | AAAGCUAAACAACCAAACUC | + |
| 2518 | 84146 | ZNF644 | intron_09 | chr1: 90918392-90935407 | chr1: 90919638-90919657 | CAAGUUAGGUAUUAAAGAAU | + |
| 2519 | 84146 | ZNF644 | intron_09 | chr1: 90918392-90935407 | chr1: 90919637-90919656 | UCAAGUUAGGUAUUAAAGAA | + |
| 2520 | 84146 | ZNF644 | intron_09 | chr1: 90918392-90935407 | chr1: 90919600-90919619 | CUUAUUAGACUCUUCUUCUA | − |
| 2521 | 84146 | ZNF644 | intron_09 | chr1: 90918392-90935407 | chr1: 90919510-90919529 | CUAAAUCUCAUUGAAGCCAC | + |
| 2522 | 84146 | ZNF644 | intron_09 | chr1: 90918392-90935407 | chr1: 90919440-90919459 | AACAUAGUUCCUUAGUCAAA | + |
| 2523 | 84146 | ZNF644 | intron_09 | chr1: 90918392-90935407 | chr1: 90919330-90919349 | CUCUUACUUAGAAUUUUUCC | − |
| 2524 | 84146 | ZNF644 | intron_09 | chr1: 90918392-90935407 | chr1: 90919311-90919330 | CAGGGAAUUUUUCACAUGGC | − |
| 2525 | 84146 | ZNF644 | intron_09 | chr1: 90918392-90935407 | chr1: 90919270-90919289 | UAACAGAAGAGUUACCCAAA | + |
| 2526 | 84146 | ZNF644 | intron_09 | chr1: 90918392-90935407 | chr1: 90919237-90919256 | CUGUAGGGAUCCAAGGGGGG | + |
| 2527 | 84146 | ZNF644 | intron_09 | chr1: 90918392-90935407 | chr1: 90919234-90919253 | UAUCUGUAGGGAUCCAAGGG | + |
| 2528 | 84146 | ZNF644 | intron_09 | chr1: 90918392-90935407 | chr1: 90919232-90919251 | UUUAUCUGUAGGGAUCCAAG | + |
| 2529 | 84146 | ZNF644 | intron_09 | chr1: 90918392-90935407 | chr1: 90919194-90919213 | GGUGCUGCUGGUCAAUUAGC | + |
| 2530 | 84146 | ZNF644 | intron_09 | chr1: 90918392-90935407 | chr1: 90919182-90919201 | AUAGCAAUCCAGGGUGCUGC | + |
| 2531 | 84146 | ZNF644 | intron_09 | chr1: 90918392-90935407 | chr1: 90919172-90919191 | CAAAGGGAUGAUAGCAAUCC | + |
| 2532 | 84146 | ZNF644 | intron_09 | chr1: 90918392-90935407 | chr1: 90919005-90919024 | GAACAGUUCACAUAAAAUUU | − |
| 2533 | 84146 | ZNF644 | intron_09 | chr1: 90918392-90935407 | chr1: 90918735-90918754 | AUGCUGGCCCUACAUUUGAA | + |
| 2534 | 84146 | ZNF644 | intron_09 | chr1: 90918392-90935407 | chr1: 90918593-90918612 | UGAUUACUGCUCCAUUAAGU | + |
| 2535 | 84146 | ZNF644 | intron_09 | chr1: 90918392-90935407 | chr1: 90918467-90918486 | UUGGUCAAUUAUUACAAUAA | + |
| 2536 | 84146 | ZNF644 | intron_09 | chr1: 90918392-90935407 | chr1: 90918448-90918467 | AUAACUUGAAAGUACUAAAU | + |
| 2537 | 84146 | ZNF644 | intron_09 | chr1: 90918392-90935407 | chr1: 90918397-90918416 | UGGUAAAGCAUUAUUUGAAA | − |
| 2538 | 84146 | ZNF644 | exon_10_nc.1 | chr1: 90918155-90918391 | chr1: 90918281-90918300 | GAAUGCAUGCUUCAGGGAAU | − |
| 2539 | 84146 | ZNF644 | exon_10_nc.1 | chr1: 90918155-90918391 | chr1: 90918244-90918263 | CUUUUUCUAACUGAUUUAGA | − |
| 2540 | 84146 | ZNF644 | exon_10_nc.1 | chr1: 90918155-90918391 | chr1: 90918209-90918228 | ACACACAUAUUUUUCUAGAG | + |
| 2541 | 84146 | ZNF644 | exon_10_c.1/nc.2 | chr1: 90918052-90918154 | chr1: 90918132-90918151 | UUUCUUUUGCUUACAAUCUA | + |
| 2542 | 84146 | ZNF644 | exon_10_c.1/nc.2 | chr1: 90918052-90918154 | chr1: 90918118-90918137 | AAGAAAUCAAGGUCAAGAUC | − |
| 2543 | 84146 | ZNF644 | exon_10_c.1/nc.2 | chr1: 90918052-90918154 | chr1: 90918074-90918093 | AUUACCUCAUGGUGCUGACG | − |
| 2544 | 84146 | ZNF644 | exon_10_c.1/nc.2 | chr1: 90918052-90918154 | chr1: 90918054-90918073 | AGGUUUACAUUCUCCGAUGC | − |
| 2545 | 84146 | ZNF644 | exon_10_c.1/nc.2 | chr1: 90918052-90918154 | chr1: 90918038-90918057 | AUAUAGGCAUAUACCUGCAU | + |
| 2546 | 84146 | ZNF644 | intron_10 | chr1: 90916991-90918051 | chr1: 90918022-90918041 | CUGAUCAGAUUUGGCAAUAU | + |
| 2547 | 84146 | ZNF644 | intron_10 | chr1: 90916991-90918051 | chr1: 90917822-90917841 | UAAUAGAUUUACAUUACUAU | + |
| 2548 | 84146 | ZNF644 | intron_10 | chr1: 90916991-90918051 | chr1: 90917799-90917818 | UGUGAUUUAUUAAAGUAGCU | − |
| 2549 | 84146 | ZNF644 | intron_10 | chr1: 90916991-90918051 | chr1: 90917375-90917394 | GGAAGAAGGCGUGACUAUGA | + |
| 2550 | 84146 | ZNF644 | intron_10 | chr1: 90916991-90918051 | chr1: 90917374-90917393 | CGGAAGAAGGCGUGACUAUG | + |
| 2551 | 84146 | ZNF644 | intron_10 | chr1: 90916991-90918051 | chr1: 90917361-90917380 | GUGAGUGUGAGUACGGAAGA | + |
| 2552 | 84146 | ZNF644 | intron_10 | chr1: 90916991-90918051 | chr1: 90917306-90917325 | AUUUAAGGAAUGCUAAACUG | − |
| 2553 | 84146 | ZNF644 | intron_10 | chr1: 90916991-90918051 | chr1: 90917184-90917203 | UCCAGUAAUCUUUGUAUAGG | + |
| 2554 | 84146 | ZNF644 | exon_11_c.1/nc.1 | chr1: 90916801-90916990 | chr1: 90916943-90916962 | CAGUCUUCCUGAACAGACAA | + |
| 2555 | 84146 | ZNF644 | exon_11_c.1/nc.1 | chr1: 90916801-90916990 | chr1: 90916945-90916964 | CCUUGUCUGUUCAGGAAGAC | − |
| 2556 | 84146 | ZNF644 | exon_11_c.1/nc.1 | chr1: 90916801-90916990 | chr1: 90916880-90916899 | ACCAUGCCAGCUCCAGUCCG | + |
| 2557 | 84146 | ZNF644 | exon_11_c.1/nc.1 | chr1: 90916801-90916990 | chr1: 90916881-90916900 | ACGGACUGGAGCUGGCAUGG | − |
| 2558 | 84146 | ZNF644 | exon_11_c.1/nc.1 | chr1: 90916801-90916990 | chr1: 90916792-90916811 | CUGGUUCUAUGAAGCUGCUU | + |
| 2559 | 84146 | ZNF644 | exon_11_nc.3 | chr1: 90916490-90916790 | chr1: 90916753-90916772 | AAUUUACAUCCAAUUCAAAC | + |
| 2560 | 84146 | ZNF644 | exon_11_nc.3 | chr1: 90916490-90916790 | chr1: 90916765-90916784 | UUUAAAUAGCCAGUUUGAAU | − |
| 2561 | 84146 | ZNF644 | exon_11_nc.3 | chr1: 90916490-90916790 | chr1: 90916688-90916707 | UACUAAAGCAGGAAAAUGGG | − |
| 2562 | 84146 | ZNF644 | exon_11_nc.3 | chr1: 90916490-90916790 | chr1: 90916616-90916635 | GUAGUCUAUAUAUUUUAUAU | − |
| 2563 | 84146 | ZNF644 | exon_11_nc.3 | chr1: 90916490-90916790 | chr1: 90916509-90916528 | CAUGUGCAACAGUUUAUUAA | + |
| 2564 | 84146 | ZNF644 | exon_11_nc.3 | chr1: 90916490-90916790 | chr1: 90916488-90916507 | AUACUUAUAGACAGACUUAU | − |
| 2565 | 84146 | ZNF644 | exon_11_nc.4 | chr1: 90916390-90916489 | chr1: 90916452-90916471 | UUGCAGUGUUACCAGAAUCA | − |
| 2566 | 84146 | ZNF644 | exon_11_nc.4 | chr1: 90916390-90916489 | chr1: 90916414-90916433 | UUUUUCUAUGCAAGUCUUGU | + |
| 2567 | 84146 | ZNF644 | exon_11_nc.6 | chr1: 90915306-90916184 | chr1: 90916156-90916175 | AAGAAAUAUGUACAUAGUUC | − |
| 2568 | 84146 | ZNF644 | exon_11_nc.6 | chr1: 90915306-90916184 | chr1: 90916138-90916157 | UCUGGAAAUUAUUUGUGGUA | − |
| 2569 | 84146 | ZNF644 | exon_11_nc.6 | chr1: 90915306-90916184 | chr1: 90915621-90915640 | AAAAUAUGGUGCAGGUGUCU | − |
| 2570 | 84146 | ZNF644 | exon_11_nc.6 | chr1: 90915306-90916184 | chr1: 90915493-90915512 | CCAUUUUAUUUCAUGUAGUA | − |
| 2571 | 84146 | ZNF644 | exon_11_nc.6 | chr1: 90915306-90916184 | chr1: 90915450-90915469 | AAAGAAUCAAUGCAUAUCCU | + |
| 2572 | 84146 | ZNF644 | exon_11_nc.6 | chr1: 90915306-90916184 | chr1: 90915354-90915373 | CCAUAGGAAAGCAAUUUCAC | + |
| 2573 | 84146 | ZNF644 | exon_11_nc.6 | chr1: 90915306-90916184 | chr1: 90915357-90915376 | CCAGUGAAAUUGCUUUCCUA | − |
| 2574 | 84146 | ZNF644 | exon_11_nc.6 | chr1: 90915306-90916184 | chr1: 90915324-90915343 | AUUUAGAGAUUUUUCUGGUA | + |

TABLE 3

| SEQ | target_ | | target_ | target_ | gRNA_target_ | | |
|---|---|---|---|---|---|---|---|
| ID | gene_ | target_ | region_ | region_ | site_ | gRNA Targeting | |
| NO | id | symbol | name | coordinates | coordinates | Domain | strand |
| 2575 | 84146 | ZNF644 | promoter1.2 | chr1: | chr1: | CUCUGCAAGACCCAAGCACU | + |
| | | | | 91022273- | 91022379- | | |
| | | | | 91023899 | 91022398 | | |
| 2576 | 84146 | ZNF644 | intron_04 | chr1: | chr1: | GAAAUUGAACUGAACCGUAA | − |
| | | | | 90982371- | 91013641- | | |
| | | | | 91020447 | 91013660 | | |
| 2577 | 84146 | ZNF644 | intron_04 | chr1: | chr1: | AAGCACAGACAUGAGAAUUG | − |
| | | | | 90982371- | 91012692- | | |
| | | | | 91020447 | 91012711 | | |
| 2578 | 84146 | ZNF644 | intron_04 | chr1: | chr1: | GGUUUUUGGAGCCUCUUUGA | − |
| | | | | 90982371- | 91011876- | | |
| | | | | 91020447 | 91011895 | | |
| 2579 | 84146 | ZNF644 | intron_04 | chr1: | chr1: | AAGACAAAUACUGAAAUCCC | + |
| | | | | 90982371- | 91011211- | | |
| | | | | 91020447 | 91011230 | | |
| 2580 | 84146 | ZNF644 | intron_04 | chr1: | chr1: | GUCAAUAUAAAAUAACUCCU | − |
| | | | | 90982371- | 90993591- | | |
| | | | | 91020447 | 90993610 | | |
| 2581 | 84146 | ZNF644 | intron_04 | chr1: | chr1: | GAGUCACCCAAAAGGAUGAA | + |
| | | | | 90982371- | 90990719- | | |
| | | | | 91020447 | 90990738 | | |
| 2582 | 84146 | ZNF644 | intron_04 | chr1: | chr1: | AUUAAAGAAAACAUAGGGGA | + |
| | | | | 90982371- | 90988931- | | |
| | | | | 91020447 | 90988950 | | |
| 2583 | 84146 | ZNF644 | intron_05 | chr1: | chr1: | AUAUUCCUAGUUCUAGUCCU | − |
| | | | | 90973214- | 90980348- | | |
| | | | | 90982309 | 90980367 | | |
| 2584 | 84146 | ZNF644 | intron_05 | chr1: | chr1: | AUUCUGAAUCUCUAUUUGCC | + |
| | | | | 90973214- | 90980058- | | |
| | | | | 90982309 | 90980077 | | |
| 2585 | 84146 | ZNF644 | intron_05 | chr1: | chr1: | GUAAAAAACUGUUAGGAUGG | − |
| | | | | 90973214- | 90979632- | | |
| | | | | 90982309 | 90979651 | | |
| 2586 | 84146 | ZNF644 | intron_05 | chr1: | chr1: | UUAGGAUAAAUAUUUCCUUA | − |
| | | | | 90973214- | 90977304- | | |
| | | | | 90982309 | 90977323 | | |
| 2587 | 84146 | ZNF644 | intron_05 | chr1: | chr1: | UUCAGUUUAAGUGUCACUUC | + |
| | | | | 90973214- | 90975071- | | |
| | | | | 90982309 | 90975090 | | |
| 2588 | 84146 | ZNF644 | intron_06 | chr1: | chr1: | UAUAAAUAUACACUGUAGUA | + |
| | | | | 90941310- | 90971044- | | |
| | | | | 90972743 | 90971063 | | |
| 2589 | 84146 | ZNF644 | intron_06 | chr1: | chr1: | AUGAGUGGAGUUAAAUUAUU | − |
| | | | | 90941310- | 90969028- | | |
| | | | | 90972743 | 90969047 | | |
| 2590 | 84146 | ZNF644 | intron_06 | chr1: | chr1: | CAUGGACUAGUAGGCAUUUU | + |
| | | | | 90941310- | 90966791- | | |
| | | | | 90972743 | 90966810 | | |
| 2591 | 84146 | ZNF644 | intron_06 | chr1: | chr1: | UCAUGGACUAGUAGGCAUUU | + |
| | | | | 90941310- | 90966790- | | |
| | | | | 90972743 | 90966809 | | |
| 2592 | 84146 | ZNF644 | intron_06 | chr1: | chr1: | GGCCAACUUCAUGGACUAGU | + |
| | | | | 90941310- | 90966782- | | |
| | | | | 90972743 | 90966801 | | |
| 2593 | 84146 | ZNF644 | intron_06 | chr1: | chr1: | UGCCUACUAGUCCAUGAAGU | − |
| | | | | 90941310- | 90966787- | | |
| | | | | 90972743 | 90966806 | | |
| 2594 | 84146 | ZNF644 | intron_06 | chr1: | chr1: | AUAAGCACAUCAGUGAUAAC | + |
| | | | | 90941310- | 90965386- | | |
| | | | | 90972743 | 90965405 | | |
| 2595 | 84146 | ZNF644 | intron_06 | chr1: | chr1: | GUCCAAGACAAAAGUACAGU | + |
| | | | | 90941310- | 90965227- | | |
| | | | | 90972743 | 90965246 | | |
| 2596 | 84146 | ZNF644 | intron_06 | chr1: | chr1: | UACCACUUGGUGGCACUAUG | − |
| | | | | 90941310- | 90964631- | | |
| | | | | 90972743 | 90964650 | | |
| 2597 | 84146 | ZNF644 | intron_06 | chr1: | chr1: | CAAUAGUAAUUUUGUUAAGG | + |
| | | | | 90941310- | 90963182- | | |
| | | | | 90972743 | 90963201 | | |
| 2598 | 84146 | ZNF644 | intron_06 | chr1: | chr1: | GCUCAAUAGUAAUUUUGUUA | + |
| | | | | 90941310- | 90963179- | | |
| | | | | 90972743 | 90963198 | | |
| 2599 | 84146 | ZNF644 | intron_06 | chr1: | chr1: | ACAUUUAACAGACAUGGAGA | + |
| | | | | 90941310- | 90953339- | | |
| | | | | 90972743 | 90953358 | | |
| 2600 | 84146 | ZNF644 | intron_06 | chr1: | chr1: | GACAAAUCAACAGCCACAGA | + |
| | | | | 90941310- | 90950625- | | |
| | | | | 90972743 | 90950644 | | |
| 2601 | 84146 | ZNF644 | intron_08 | chr1: | chr1: | UUAGGCUUUCUGAUGAAGGC | + |
| | | | | 90935529- | 90935987- | | |
| | | | | 90937484 | 90936006 | | |
| 2602 | 84146 | ZNF644 | intron_09 | chr1: | chr1: | UGGCUCUUAUUGACUUAAUU | − |
| | | | | 90918392- | 90935197- | | |
| | | | | 90935407 | 90935216 | | |
| 2603 | 84146 | ZNF644 | intron_09 | chr1: | chr1: | UACCAACCCCUAAGAGAAAG | + |
| | | | | 90918392- | 90930531- | | |
| | | | | 90935407 | 90930550 | | |
| 2604 | 84146 | ZNF644 | intron_09 | chr1: | chr1: | ACGUUAAUAUAUUGCACUAG | + |
| | | | | 90918392- | 90929877- | | |
| | | | | 90935407 | 90929896 | | |
| 2605 | 84146 | ZNF644 | intron_09 | chr1: | chr1: | UAGUCUAAGCCUCCUUUUGA | − |
| | | | | 90918392- | 90929209- | | |
| | | | | 90935407 | 90929228 | | |
| 2606 | 84146 | ZNF644 | intron_09 | chr1: | chr1: | AGGUUAAUUCUUGUUAUCCA | − |
| | | | | 90918392- | 90924801- | | |
| | | | | 90935407 | 90924820 | | |
| 2607 | 84146 | ZNF644 | intron_09 | chr1: | chr1: | UCCCCUCUACUUUGAAAUAG | − |
| | | | | 90918392- | 90924000- | | |
| | | | | 90935407 | 90924019 | | |
| 2608 | 84146 | ZNF644 | intron_09 | chr1: | chr1: | CUACCCUCCACCCUUAAGGU | + |
| | | | | 90918392- | 90922737- | | |
| | | | | 90935407 | 90922756 | | |
| 2609 | 84146 | ZNF644 | intron_09 | chr1: | chr1: | UAUACUGUGCCUCUUACUUU | − |
| | | | | 90918392- | 90922325- | | |
| | | | | 90935407 | 90922344 | | |
| 2610 | 84146 | ZNF644 | intron_09 | chr1: | chr1: | AAACAACUCUGUUAAAGCUU | − |
| | | | | 90918392- | 90921302- | | |
| | | | | 90935407 | 90921321 | | |
| 2611 | 84146 | ZNF644 | intron_09 | chr1: | chr1: | GGCAGAAUAGAGCAGGCAUU | + |
| | | | | 90918392- | 90920184- | | |
| | | | | 90935407 | 90920203 | | |
| 2612 | 84146 | ZNF644 | intron_09 | chr1: | chr1: | AUUUAUCUGUAGGGAUCCAA | + |
| | | | | 90918392- | 90919231- | | |
| | | | | 90935407 | 90919250 | | |
| 2613 | 84146 | ZNF644 | exon_11_ | chr1: | chr1: | AAUAAACAGAGCCUUGAUUC | + |
| | | | nc.4 | 90916390- | 90916438- | | |
| | | | | 90916489 | 90916457 | | |

aacagcauagcaaguuaaaauaaggcuaguccguuaucaacuugaaaaaguggcaccgagucggugcuuuuuuu (SEQ ID NO: 3109)

1. A guide RNA (gRNA) molecule comprising a tracr and crRNA,
wherein the crRNA comprises a targeting domain that is complementary with a target sequence of a zinc finger protein 644 (ZNF644), wherein the targeting domain comprises any one of SEQ ID NO: 1 to SEQ ID NO: 2613.

2-4. (canceled)

5. The gRNA molecule of claim 1, wherein the targeting domain comprises any one of SEQ ID NO: 2634, SEQ ID NO: 2635, SEQ ID NO: 2636, SEQ ID NO: 2637, SEQ ID NO: 2638, SEQ ID NO: 2639, SEQ ID NO: 2640, or SEQ ID NO: 2641.

6-12. (canceled)

13. The gRNA molecule of claim 1, comprising:
(a) SEQ ID NO: 3123;
(b) SEQ ID NO: 3159; or
(c) any of (a) or (b), above, further comprising, at the 3′ end, 1, 2, 3, 4, 5, 6 or 7 uracil (U) nucleotides;
wherein the sequence of any of (a) to (c) is disposed 3′ to the targeting domain.

14. The gRNA molecule of claim 1, comprising:
(a) a tracr comprising SEQ ID NO: 3152; or
(b) a tracr comprising SEQ ID NO: 3109 or 3174;

15-20. (canceled)

21. A composition comprising:
1) the gRNA molecule of claim 1 and a Cas9 molecule;
2) the gRNA molecule of claim 1 and a nucleic acid comprising a nucleotide sequence encoding a Cas9 molecule;
3) a nucleic acid comprising a nucleotide sequence encoding the gRNA molecule of claim 1 and a Cas9 molecule;
4) a nucleic acid comprising a nucleotide sequence encoding the gRNA molecule of claim 1 and a nucleic acid comprising a nucleotide sequence encoding a Cas9 molecule; or
5) any of 1) to 4), above, and a template nucleic acid; or
6) any of 1) to 4) above, and a nucleic acid comprising a nucleotide sequence encoding a template nucleic acid.

22. The composition of claim 21, wherein the Cas9 molecule:
a) is an active or inactive S. pyogenes Cas9; or
(b) comprises SEQ ID NO: 3133, SEQ ID NO: 3161, SEQ ID NO: 3162, SEQ ID NO: 3163, SEQ ID NO: 3164, SEQ ID NO: 3165, SEQ ID NO: 3166, SEQ ID NO: 3167, SEQ ID NO: 3168, SEQ ID NO: 3169, SEQ ID NO: 3170, SEQ ID NO: 3171, or SEQ ID NO: 3172.

23-26. (canceled)

27. A nucleic acid that encodes the gRNA molecule of claim 1.

28. A vector comprising the nucleic acid of claim 27.

29. A method of altering a cell at or near a target sequence within said cell, comprising contacting said cell with:
1) the gRNA molecule of claim 1 and a Cas9 molecule;
2) the gRNA molecule of claim 1 and a nucleic acid comprising a nucleotide sequence encoding a Cas9 molecule;
3) a nucleic acid comprising a nucleotide sequence encoding the gRNA molecule of claim 1 and a Cas9 molecule;
4) a nucleic acid comprising a nucleotide sequence encoding the gRNA molecule of claim 1 and a nucleic acid comprising a nucleotide sequence encoding a Cas9 molecule;
5) any of 1) to 4), above, and a template nucleic acid;
6) any of 1) to 4) above, and a nucleic acid comprising a nucleotide sequence encoding a template nucleic acid;
7) the composition of claim 21; or
8) the vector of claim 28.

30-36. (canceled)

37. A cell, altered by the method of claim 29.

38. A cell, comprising the gRNA molecule of claim 1.

39-42. (canceled)

43. The cell of claim 37, wherein the cell is:
(a) an animal cell,
(b) a mammalian, primate, or human cell,
(c) a cell obtained from a patient suffering from a hemoglobinopathy,
(d) a cell obtained from a patient suffering from sickle cell disease or a thalassemia
(e) an HSPC, a CD34+ HSPC, or a CD34+CD90+ HSPC,
(f) a cell that has been isolated from bone marrow, peripheral blood, or umbilical cord blood, or
(g) a cell that is autologous or allogeneic with respect to a patient to be administered said cell.

44-46. (canceled)

47. A population of cells comprising the cell of claim 37, wherein at least about 50% of the cells of the population are a cell according to claim 37.

48. (canceled)

49. The population of cells of claim 47, comprising:
1) at least 1e6 CD34+ cells/kg body weight of the patient to whom the cells are to be administered;
2) at least 2e6 CD34+ cells/kg body weight of the patient to whom the cells are to be administered;
3) at least 3e6 CD34+ cells/kg body weight of the patient to whom the cells are to be administered;
4) at least 4e6 CD34+ cells/kg body weight of the patient to whom the cells are to be administered; or
5) from 2e6 to 10e6 CD34+ cells/kg body weight of the patient to whom the cells are to be administered.

50-53. (canceled)

54. A composition comprising the cell of claim 37 and a pharmaceutically acceptable medium.

55. A method of treating a hemoglobinopathy, comprising administering to a patient the cell of claim 37.

56. A method of increasing fetal hemoglobin expression in a mammal, comprising administering to a patient the cell of claim 37.

57. The method of claim 55, wherein the hemoglobinopathy is beta-thalassemia or sickle cell disease.

58. A method of increasing fetal hemoglobin expression in a subject comprising administering to the subject a composition that reduces ZNF644 gene expression and/or ZNF644 protein activity, wherein the composition comprises a small molecule compound, siRNA, shRNA, antisense oligonucleotide (ASO), miRNA, anti-microRNA oligonucleotide (AMO) or any combination thereof.

59-89. (canceled)

90. A method of treating a hemoglobinopathy in a subject, comprising administering to the subject a composition that reduces ZNF644 gene expression and/or ZNF644 protein activity, wherein the composition that reduces ZNF644 gene expression and/or ZNF644 protein activity comprises a small molecule compound, siRNA, shRNA, ASO, miRNA, AMO, or any combination thereof.

91. (canceled)
